(19) United States
(12) Patent Application Publication
(10) Pub. No.: US 2024/0166705 A1
(43) Pub. Date: May 23, 2024

(54) IL18-FC FUSION PROTEINS

(71) Applicant: Xencor, Inc., Pasadena, CA (US)

(72) Inventors: Alex NISTHAL, Monrovia, CA (US); John DESJARLAIS, Altadena, CA (US); Gregory MOORE, Azusa, CA (US); Sung-Hyung LEE, San Gabriel, CA (US); Matthew Adam DRAGOVICH, Rancho Cucamonga, CA (US)

(21) Appl. No.: 18/503,935

(22) Filed: Nov. 7, 2023

Related U.S. Application Data
(60) Provisional application No. 63/382,590, filed on Nov. 7, 2022.
(60) Provisional application No. 63/387,650, filed on Dec. 15, 2022.
(60) Provisional application No. 63/387,913, filed on Dec. 16, 2022.
(60) Provisional application No. 63/478,652, filed on Jan. 5, 2023.
(60) Provisional application No. 63/481,343, filed on Jan. 24, 2023.
(60) Provisional application No. 63/502,279, filed on May 15, 2023.
(60) Provisional application No. 63/502,344, filed on May 15, 2023.
(60) Provisional application No. 63/590,872, filed on Oct. 17, 2023.

(51) Int. Cl. C07K 14/54; C07K 16/28; C07K 16/30

(52) U.S. Cl. CPC C07K 14/54; C07K 16/28; C07K 16/2803; C07K 16/2809; C07K 16/2818; C07K 16/2827; C07K 16/2863; C07K 16/2866; C07K 16/2878; C07K 16/2887; C07K 16/2896; C07K 16/30; C07K 2317/31; C07K 2317/622; C07K 2319/30

(57) ABSTRACT

Provided herein are variant human IL18 proteins including proteins and constructs including such. Various variant IL18 proteins with modifications to reduce heterogeneity and/or reduce affinity/potency and/or improve stability and/or improve production yield are also described. Also provided are IL18-Fc fusion proteins that include an empty-Fc domain and an IL18 protein connected to another Fc domain. Also provided herein are IL18Fc fusion proteins that bind CD3 and a tumor target antigen.

### CROSS REFERENCE TO RELATED APPLICATIONS

This application claims the benefit of U.S. Provisional Patent Application No. 63/382,590, filed Nov. 7, 2022, U.S. Provisional Patent Application No. 63/387,650, filed Dec. 15, 2022, U.S. Provisional Patent Application No. 63/387,913, filed Dec. 16, 2022, U.S. Provisional Patent Application No. 63/478,652, filed Jan. 5, 2023, U.S. Provisional Patent Application No. 63/481,343, filed Jan. 24, 2023, U.S. Provisional Patent Application No. 63/502,279 filed May 15, 2023, U.S. Provisional Patent Application No. 63/502,344 filed May 15, 2023, and U.S. Provisional Patent Application No. 63/590,872 filed Oct. 17, 2023, which are hereby incorporated by reference in their entirety.

### SEQUENCE LISTING

The instant application contains a Sequence Listing which has been submitted electronically in XML format and is hereby incorporated by reference in its entirety. The XML copy, created on Nov. 27, 2023, is named 51096_4018_US_SL.xml and is 4,879,262 bytes in size.

### BACKGROUND

In order for the immune system to mount an effective anti-tumor response, two things must occur. T cells in the tumor environment must first engage antigenic tumor peptides presented by major histocompatibility complexes (MHC) on tumor cells. Next, the T cells must be induced by cytokines such as IL-15 and IL-2 to produce costimulatory cytokines such as IFNγ. Recognition of tumor peptides alone in the absence of cytokine induction leads to T cells becoming anergic, thereby leading to tolerance. Accordingly, a very promising approach in cancer immunotherapy is cytokine-based treatments. For example, IL-2 has been approved for use in patients with metastatic renal-cell carcinoma and malignant melanoma.

IL18 is proinflammatory cytokine that exerts cell signaling upon binding to the IL18 receptor IL18R1 and the IL18 receptor accessory protein (IL18RAP) to form a ternary signaling complex which activates NF-kappa-B, and in turn activates synthesis of inflammatory mediators. The activity of IL18 can be suppressed by the IL18 binding protein (IL18BP) which binds to IL18 and prevents it from binding to the IL18 receptor. Recombinant IL18 is a promising cytokine-treatment due to its broad effect in activating the immune system as IL18 signaling contributes to cytokine production and immune response by Th1 and Th2 lymphocytes. Thus, there remains a need for novel IL18 based compositions for the treatment of cancers.

### BRIEF SUMMARY

In some aspects, provided herein is a construct comprising a variant human IL18 protein as compared to SEQ ID NO: 2, wherein the variant comprises an amino acid modification selected from the group including: K53T/M60K, 4CS/S10C/K53T/M60K/N155C, 4CS/S10C/K53T/V153K/N155C, 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/I81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, wherein 4CS comprises the amino acid substitutions C38S/C68S/C76S/C127S, and wherein the variant human IL18 protein has reduced binding to IL18BP.

In some embodiments, the variant human IL18 protein has amino acid substitutions selected from 4CS/S10C/K53T/M60K/N155C and 4CS/S10C/K53T/V153K/N155C. In some embodiments, the construct comprises the amino acid sequence of SEQ ID NO: 29.

In some aspects, provided herein is a construct comprising: (a) a first antigen binding domain (ABD) that binds to the extracellular domain (ECD) of a human tumor target antigen (TTA); (b) a second antigen binding domain (ABD) that binds to the extracellular domain (ECD) of human CD3ε; and (c) a variant human IL18 protein, wherein the variant human IL18 protein has reduced binding to TL-BP.

In some embodiments, the construct comprises: (a) a first monomer comprising from N-terminus to C-terminus: a first variable heavy domain (VH1), an scFv, and a first Fc domain; (b) a second monomer comprising from N-terminus to C-terminus: the variant human IL18 protein and a second Fc domain; and (c) a third monomer comprising from N-terminus to C-terminus: a first variable light domain (VL1) and a constant light domain, wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

In some embodiments, the construct comprises: (a) a first monomer comprising from N-terminus to C-terminus: an scFv and a first Fc domain; (b) a second monomer comprising from N-terminus to C-terminus: the variant human IL18 protein, a first variable heavy domain (VH1), and a second Fc domain; and (c) a third monomer comprising from N-terminus to C-terminus: a first variable light domain (VL1) and a constant light domain, wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

In some embodiments, the construct comprises: (a) a first monomer comprising from N-terminus to C-terminus: the variant human IL18 protein, an scFv, and a first Fc domain; (b) a second monomer comprising from N-terminus to C-terminus: a first variable heavy domain (VH1) and a second Fc domain; and (c) a third monomer comprising from N-terminus to C-terminus: a first variable light domain (VL1) and a constant light domain, wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

In some embodiments, the construct comprises: a) a first monomer comprising from N-terminus to C-terminus: a first variable heavy domain (VH1), a first Fc domain, and an scFv; (b) a second monomer comprising from N-terminus to C-terminus: the variant human IL18 protein and a second Fc domain; and (c) a third monomer comprising from N-terminus to C-terminus: a first variable light domain (VL1) and a constant light domain, wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

In some embodiments, the construct comprises: (a) a first monomer comprising from N-terminus to C-terminus: a first variable heavy domain (VH1), an scFv, and a first Fc domain; (b) a second monomer comprising from N-terminus to C-terminus: the variant human IL18 protein, a second variable heavy domain (VH2), and a second Fc domain; and (c) a third monomer comprising from N-terminus to C-terminus: a first variable light domain (VL1) and a constant light domain, wherein the VH1 and the VL1 form the first ABD that binds to the ECD of the human TTA, and wherein the VH2 and the VL1 form another ABD to the ECD of the human TTA, and wherein the scFv comprises a third variable heavy domain (VH3), a scFv linker, and a second variable light domain (VL2), wherein the VH3 and the VL2 together form the second ABD that binds to the ECD of human CD3ε.

In some embodiments, the construct comprises: (a) a first monomer comprising from N-terminus to C-terminus: the variant human IL18 protein, an scFv, and a first Fe domain; (b) a second monomer comprising from N-terminus to C-terminus: a first variable heavy domain (VH1), a second variable heavy domain (VH2), and a second Fc domain; and (c) a third monomer comprising from N-terminus to C-terminus: a first variable light domain (VL1) and a constant light domain, wherein the VH1 and the VL1 form the first ABD that binds to the ECD of the human TTA, and wherein the VH2 and the VL1 form another ABD that binds to the ECD of the human TTA, and wherein the scFv comprises a third variable heavy domain (VH3), a scFv linker, and a second variable light domain (VL2), wherein the VH3 and the VL2 together form the second ABD that binds to the ECD of human CD3ε.

In some embodiments, the construct comprises: (a) a first monomer comprising from N-terminus to C-terminus: the variant human IL18 protein, a first variable heavy domain (VH1), an scFv, and a first Fc domain; (b) a second monomer comprising a second Fc domain; and (c) a third monomer comprising from N-terminus to C-terminus: a first variable light domain (VL1) and a constant light domain, wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

In some embodiments, the construct comprises: (a) a first monomer comprising from N-terminus to C-terminus: a first variable heavy domain (VH1), an scFv, and a first Fc domain; (b) a second monomer comprising a second Fc domain; and (c) a third monomer comprising from N-terminus to C-terminus: the variant human IL18 protein, a first variable light domain (VL1), and a constant light domain, wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

In some embodiments, the variant human IL18 protein has amino acid modification selected from the group including: K53T/M60K, 4CS/S10C/K53T/M60K/N155C, 4CS/S10C/K53T/V153K/N155C, 4CS/E6Q/S10C/K53D/N111T/N155C, 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/SOC/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, and 4CS/E6Q/S10C/K53D/N111T/N155C, wherein 4CS comprises the amino acid substitutions C38S/C68S/C76S/C127S.

In some embodiments of any of the constructs, the human TTA is selected from the group including: EGFR, Trop2, CD20, B7H3, FLT3, CD19, CD123, CD22, CD38, CEA, MSLN, BCMA, CAIX, CLDN18.2, HER2, PD-1, and ANO1.

In some embodiments of any of the constructs, the first ABD that binds to the ECD of human EGFR comprises a variable heavy domain and variable light domain pair selected from those depicted in FIGS. 150A-150C as SEQ ID NOS: 3386-3409.

In some embodiments of any of the constructs, the ABD that binds to the ECD of human Trop2 comprises a variable heavy domain and variable light domain pair selected from those depicted in FIGS. 157A-157R as SEQ ID NOS: 3857-4018.

In some embodiments of any of the constructs, the first ABD that binds to the ECD of human CD20 comprises a variable heavy domain and variable light domain pair selected from those depicted in FIG. 153A as SEQ ID NOS: 3432-3441.

In some embodiments of any of the constructs, the ABD that binds to the ECD of human B7H3 comprises a variable heavy domain and variable light domain pair selected from those depicted in FIGS. 146, 147, 148A-148H, and 149A-149R as SEQ ID NOS: 2848-3385.

In some embodiments of any of the constructs, the first ABD that binds to the ECD of human FLT3 comprises a variable heavy domain and variable light domain pair selected from those depicted in FIG. 155 as SEQ ID NOS: 3468-3475.

In some embodiments of any of the constructs, the first ABD that binds to the ECD of human CD19 comprises a variable heavy domain and variable light domain pair selected from those depicted in FIG. 152 as SEQ ID NOS: 3424-3431.

In some embodiments of any of the constructs, the first ABD that binds to the ECD of human CD123 comprises a variable heavy domain and variable light domain pair selected from those depicted in FIG. 154B as SEQ ID NOS: 3464-3467.

In some embodiments of any of the constructs, the first ABD that binds to the ECD of human CEA comprises a variable heavy domain and variable light domain pair selected from those depicted in FIGS. 158A-158C as SEQ ID NOS: 4019-4046.

In some embodiments of any of the constructs, the first ABD that binds to the ECD of human MSLN comprises a variable heavy domain and variable light domain pair selected from those depicted in FIGS. 156A-156Z and 156AA-156FF as SEQ ID NOS: 3467-3856.

In some embodiments of any of the constructs, the first ABD that binds to the ECD of human BCMA comprises a variable heavy domain and variable light domain pair selected from those depicted in FIGS. 160A-160C as SEQ ID NOS: 4054-4115.

In some embodiments of any of the constructs, the first ABD that binds to the ECD of human CAIX comprises a variable heavy domain and variable light domain pair selected from those depicted in FIG. 154A as SEQ ID NOS: 3462-3463.

In some embodiments of any of the constructs, the first ABD that binds to the ECD of human CLDN18.2 comprises a variable heavy domain and variable light domain pair selected from those depicted in FIG. 159 as SEQ ID NOS: 4047-4053.

In some embodiments of any of the constructs, the first ABD that binds to the ECD of human HER2 comprises a variable heavy domain and variable light domain pair selected from those depicted in FIGS. 151A-151B as SEQ ID NOS: 3410-3423.

In some embodiments of any of the constructs, the first ABD that binds to the ECD of human PD-1 comprises a variable heavy domain and variable light domain pair selected from those depicted in FIGS. 161A-161G as SEQ ID NOS: 4116-4155.

In some embodiments of any of the constructs, the second ABD that binds to the ECD of human CD3F comprises a variable heavy domain and variable light domain pair selected from those depicted in FIGS. 120A-120F as SEQ ID NOS: 1545-1664.

### BRIEF DESCRIPTION OF THE DRAWINGS

FIGS. 1A-1B depict the sequences for human IL18, its receptors, and IL18 binding protein.

FIGS. 2A-2B depict the sequences for mouse IL18 and its receptors to facilitate investigation of IL18 fusion proteins of the invention in preclinical studies.

FIGS. 3A-3B depict the sequences for cynomolgus IL18 and its receptors to facilitate investigation of IL18 fusion proteins of the invention in preclinical studies.

FIGS. 4A-4E depict useful pairs of Fc heterodimerization variant sets (including skew and pI variants). Variants without a corresponding “monomer 2” are pI variants which can be used alone on either monomer.

FIG. 5 depicts a list of isosteric variant antibody constant regions and their respective substitutions. pI_(−) indicates lower pI variants, while pI_(+) indicates higher pI variants. These can be optionally and independently combined with other heterodimerization variants of the inventions (and other variant types as well, as outlined herein).

FIG. 6 depicts useful ablation variants that ablate FcγR binding (sometimes referred to as “knock outs” or “KO” variants). Generally, ablation variants are found on both monomers, although in some cases they may be on only one monomer.

FIG. 7 shows particularly useful embodiments of “non-cytokine”/“non-Fv” components of the IL18 fusions of the invention.

FIG. 8 depicts a number of exemplary domain linkers. In some embodiments, these linkers find use linking an IL18 monomer to an Fc chain. In other embodiments, these linkers find use linking a variable heavy region to an Fc chain (optionally via a CH1 region as depicted in FIG. 10). While the “hinge” based domain linkers in this Figure are based on IgG1 hinge, hinge regions from other IgG isotypes may also be used. In the case of IgG2, the hinge sequence may include C219S and/or C220S substitutions. Additionally, each of these domain linkers may be used in multiples (e.g., EAAAKEAAAK; SEQ ID NO:775) or in combination (e.g., EAAAKEPKSSDKTHTCPPCP; SEQ ID NO:776).

FIGS. 9A-9E show the sequences of several useful heterodimeric IL18 fusion backbones based on human IgG, without the cytokine, Fv sequences, or domain linkers. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants, N297A variant that removes glycosylation. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants, N297S variant that removes glycosylation. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants, M428L/N434S Xtend variants. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants. Heterodimeric Fc backbone 13 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 14 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356E/358M allotype), and includes the L368D/K370S skew variants, the Q295E/N384D/Q418E/N421D pI variants, and H435R/Y436 rapid purification variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 16 is based on human IgG1 (356E/358M allotype), and includes the L368D/K370S skew variants and the Q295E/N384D/Q418E/N421D pI variants on a first heterodimeric Fc chain, the S364K/E357Q skew variants and H435R/Y436 rapid purification variants on a second heterodimeric Fc chain, and the E233P/L234V/L235A/G236del/S267K ablation variants on both chains. Heterodimeric Fc backbone 17 is based on human IgG1 (356E/358M allotype), and includes the L368D/K370S skew variants, the Q295E/N384D/Q418E/N421D pI variants, and H435R/Y436 rapid purification variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 18 is based on human IgG1 (356E/358M allotype), and includes the L368D/K370S skew variants and the Q295E/N384D/Q418E/N421D pI variants on a first heterodimeric Fc chain, the S364K/E357Q skew variants and H435R/Y436 rapid purification variants on a second heterodimeric Fc chain, and the E233P/L234V/L235A/G236del/S267K ablation variants and M428L/N434S Xtend variants on both chains.

As will be appreciated by those in the art and outlined below, these sequences can be used with any IL18 fusion formats requiring a heterodimeric Fc region. It should be noted that the backbones may further comprise deletion of K447 (i.e. K447_or K447del) on one or both chains. These sequences can also be used with any of the IL18×Fab-Fc fusions of the invention. In targeted IL-10 fusion formats which include a variable heavy domain covalently linked to the Fc, the variable heavy domain may be covalently linked to the Fc domain by a corresponding CH1 domain (as depicted in FIG. 10) and domain linkers (as depicted in FIG. 8). Additionally, each of these backbone sequences may include the H435R/Y436F variants on monomer 1 or monomer 2 to ablate Protein A binding.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure.

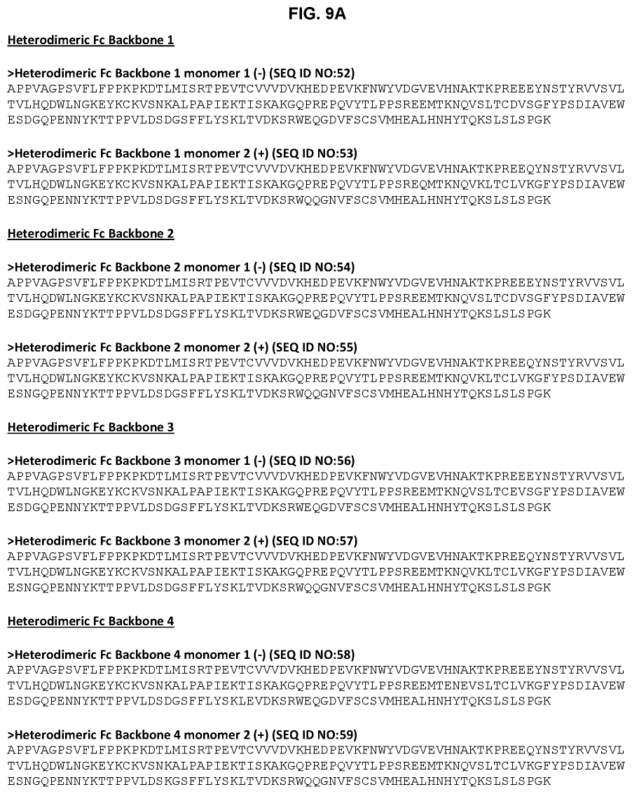

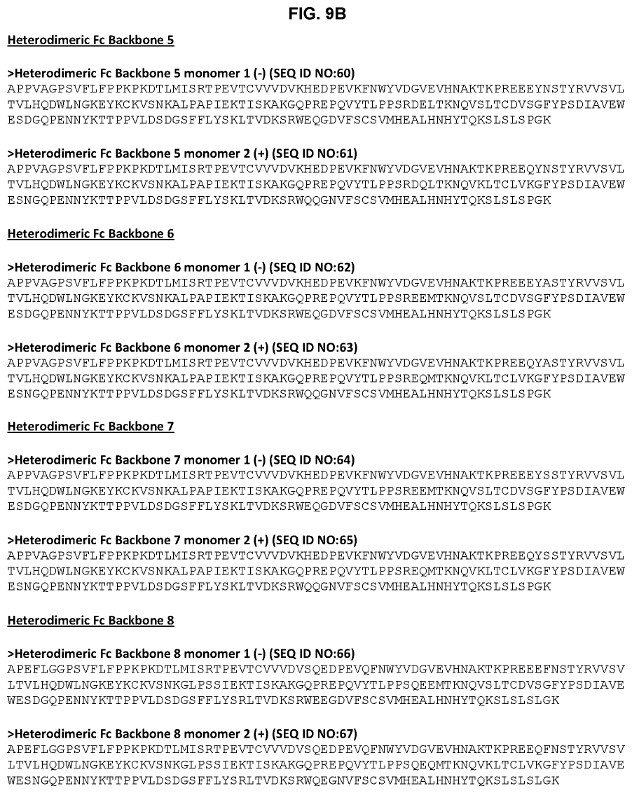

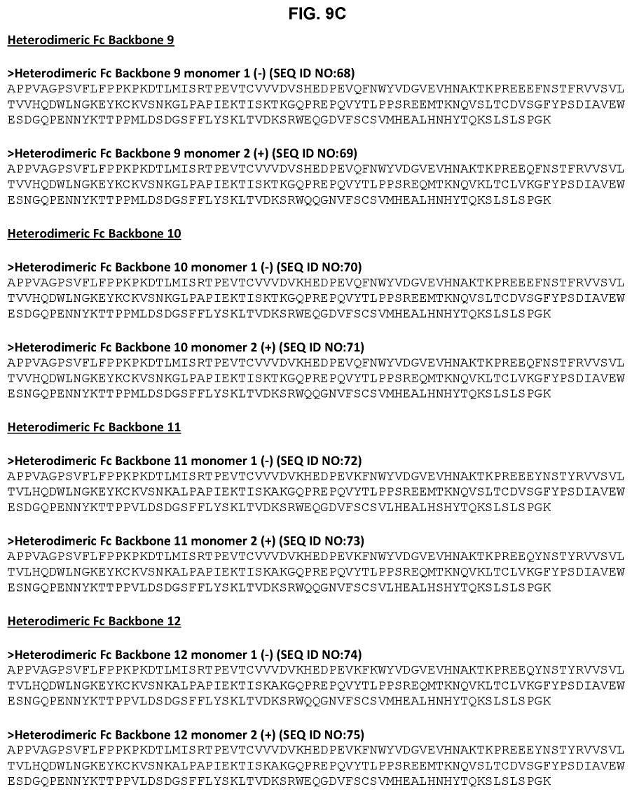

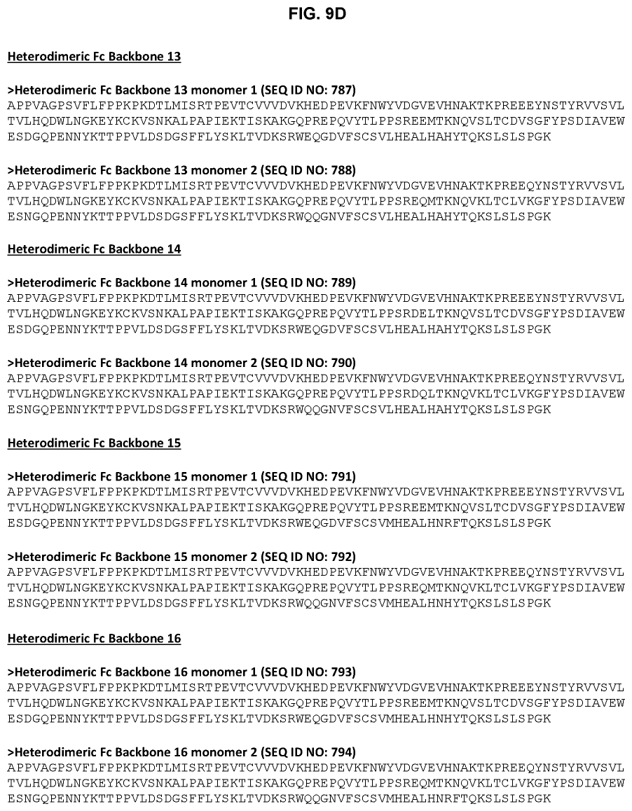

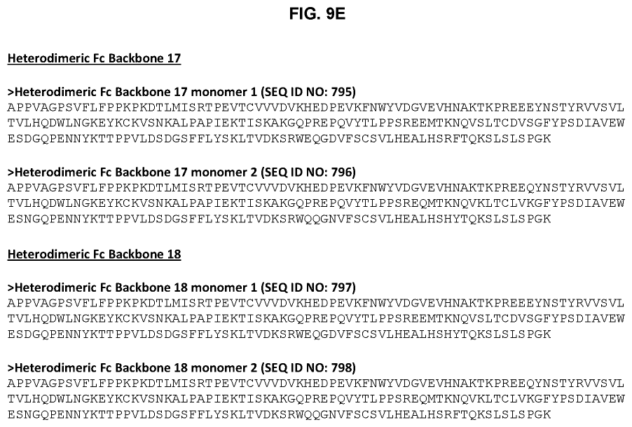

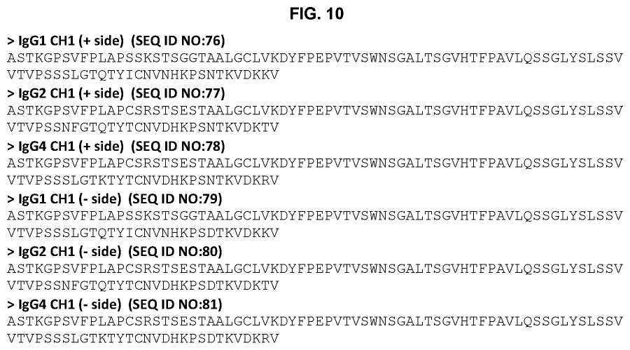

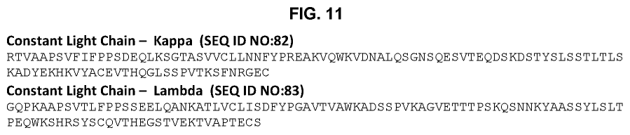

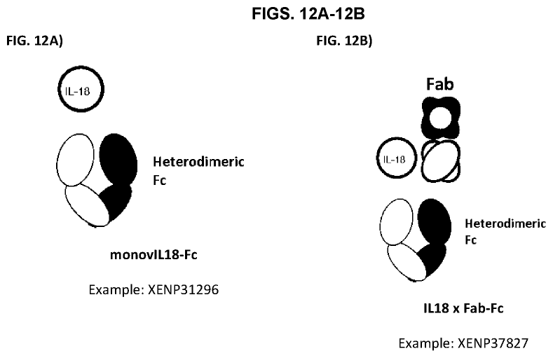

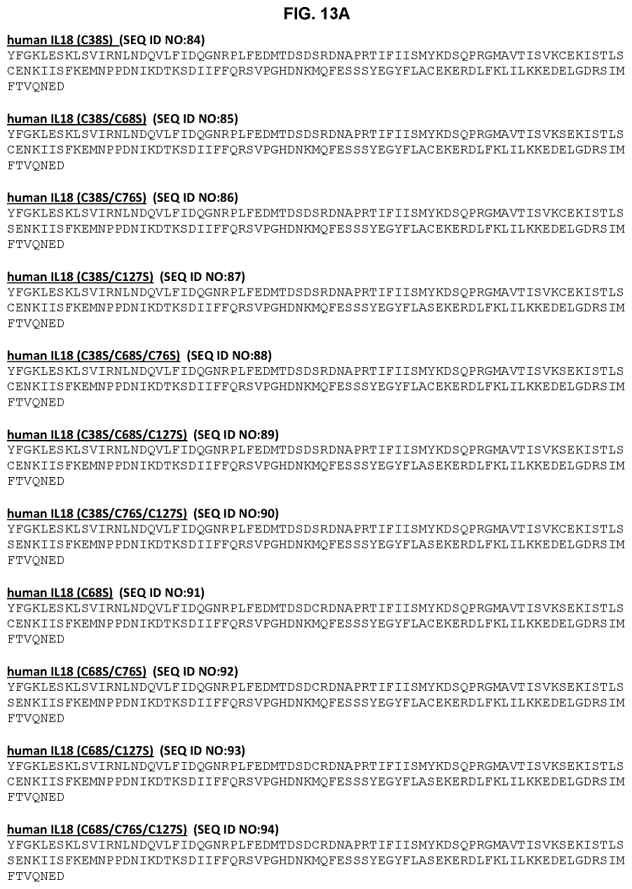

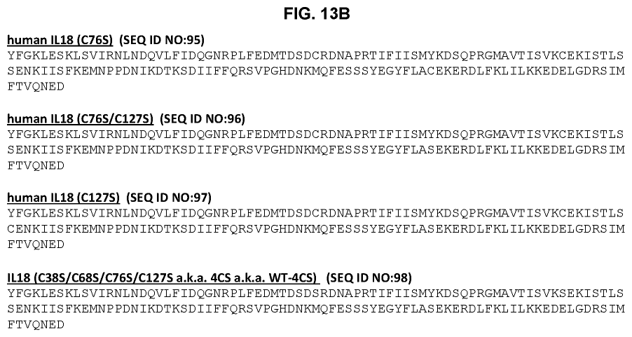

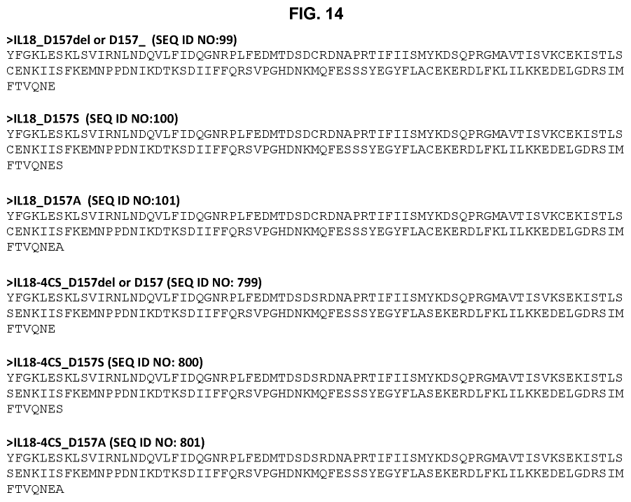

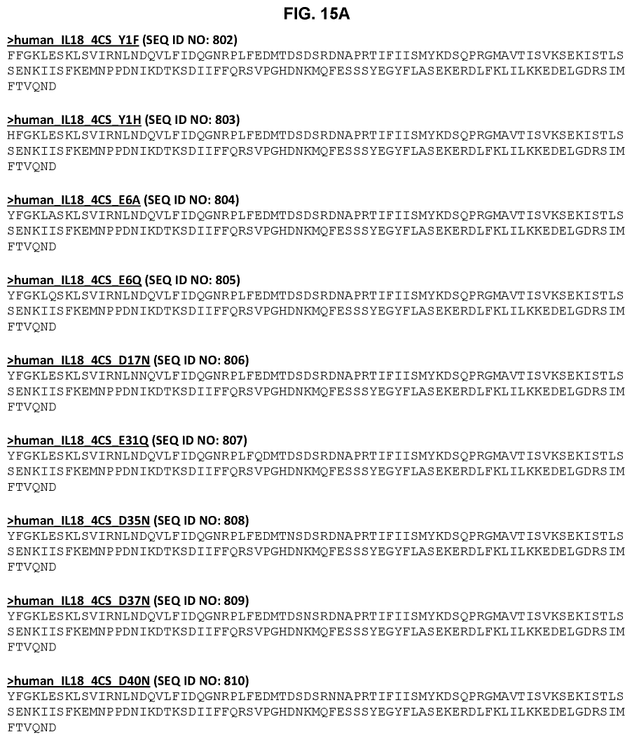

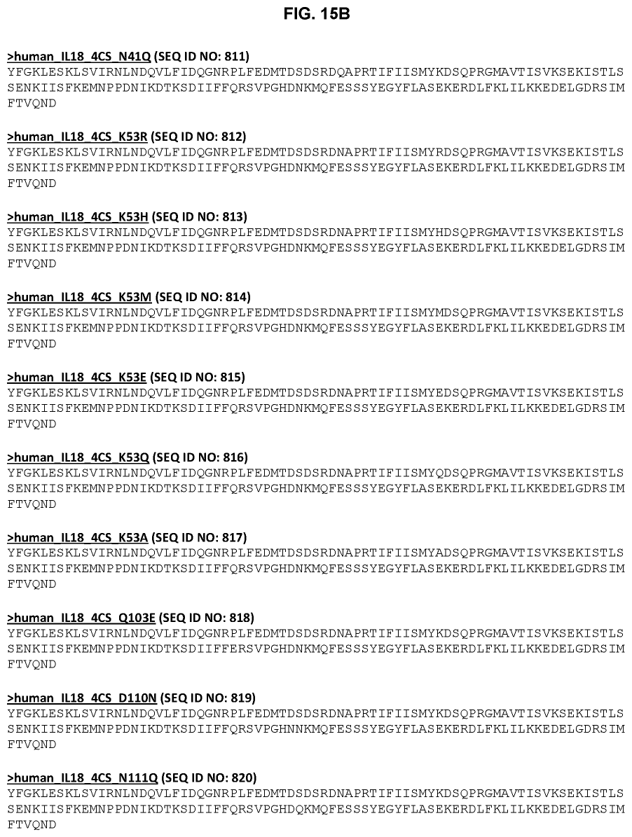

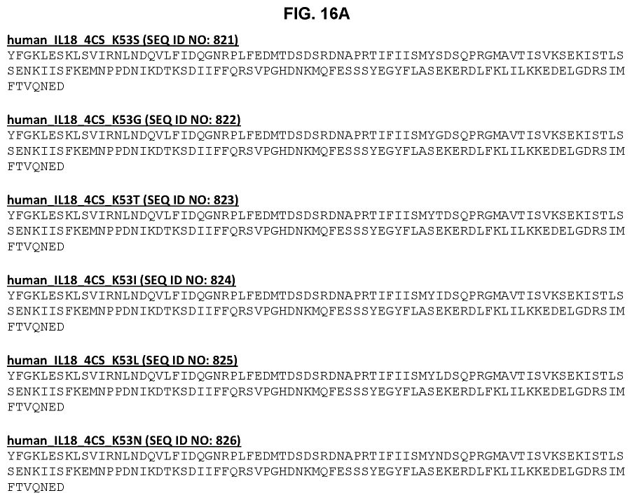

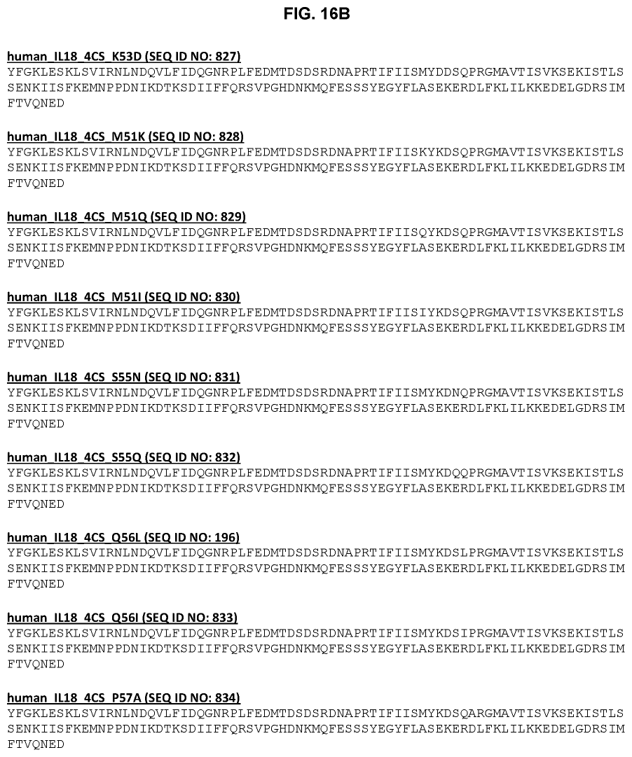

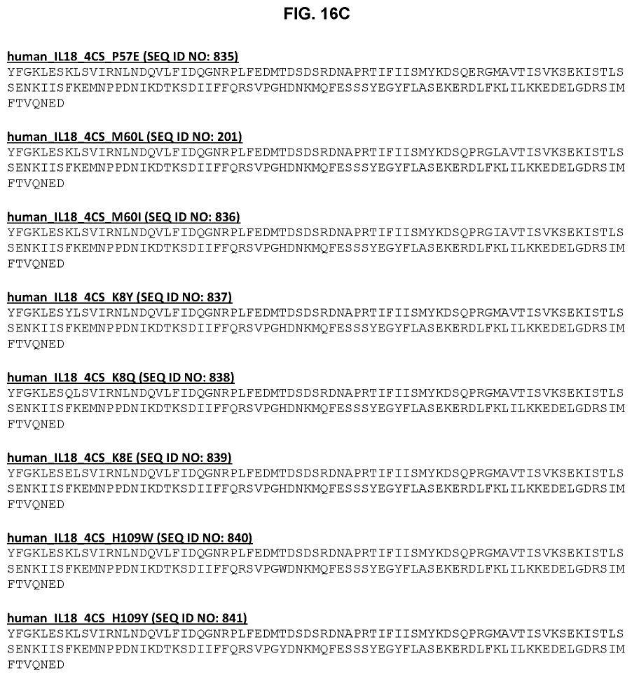

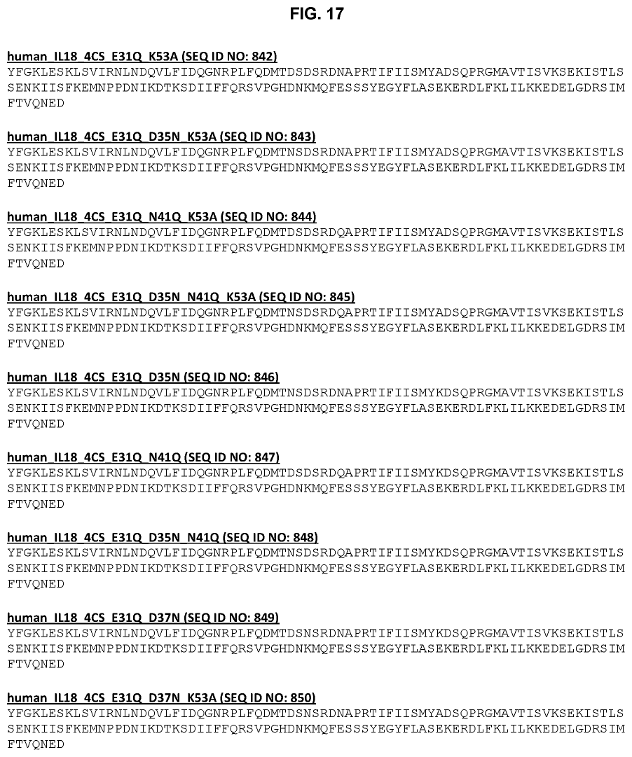

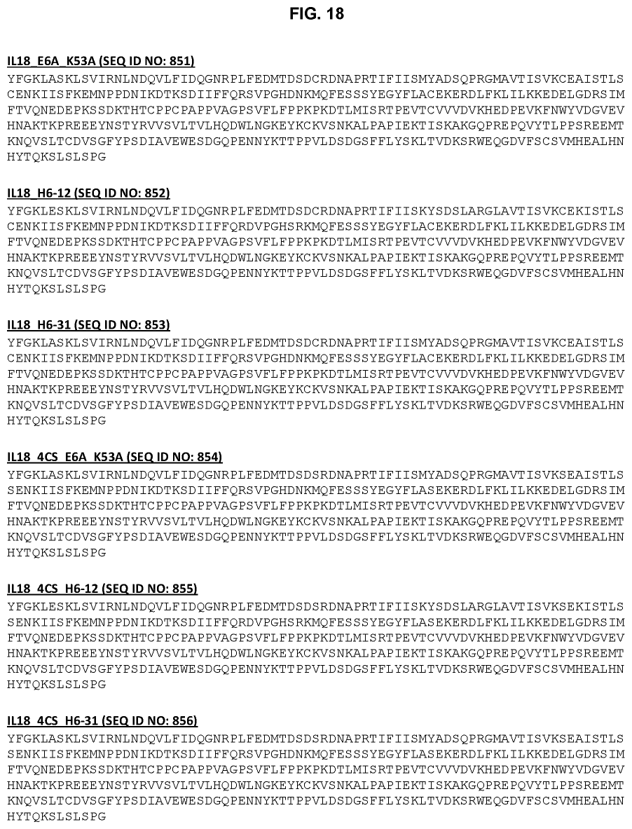

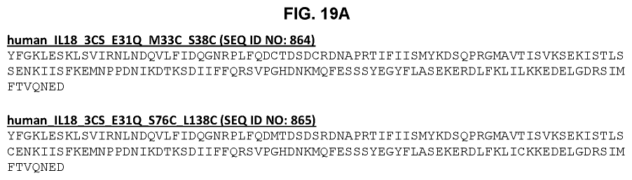

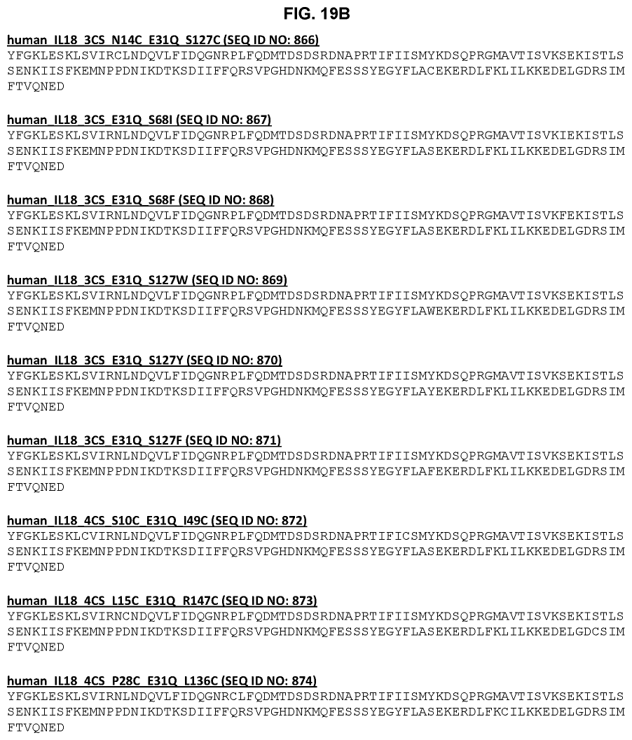

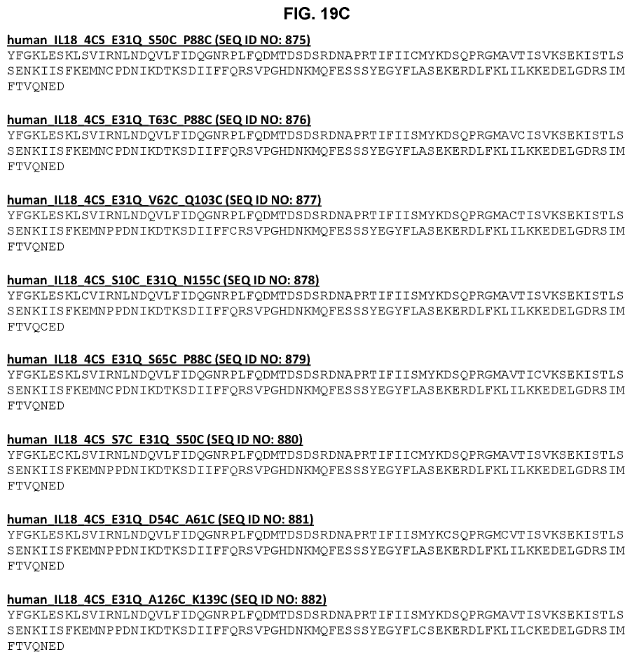

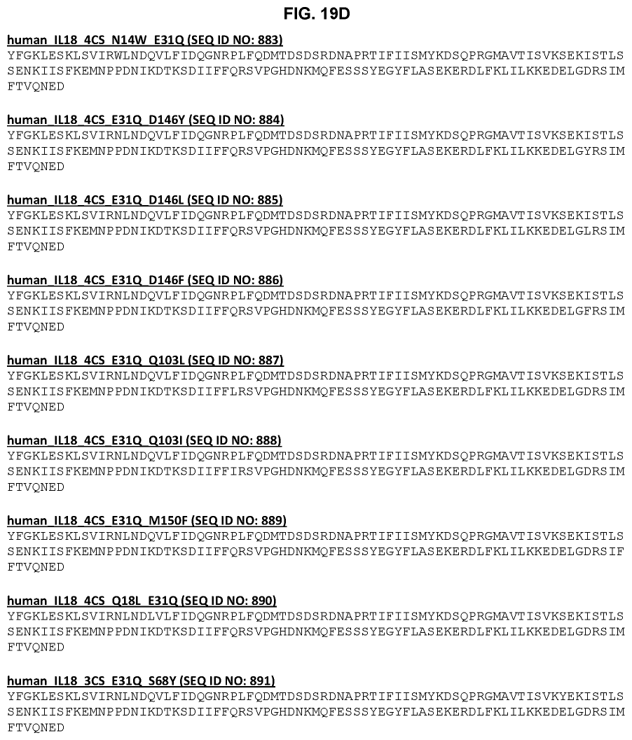

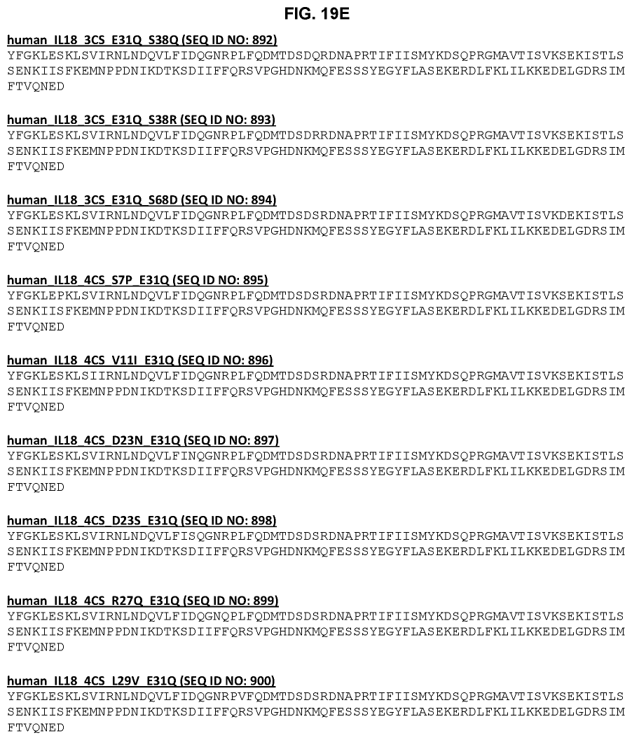

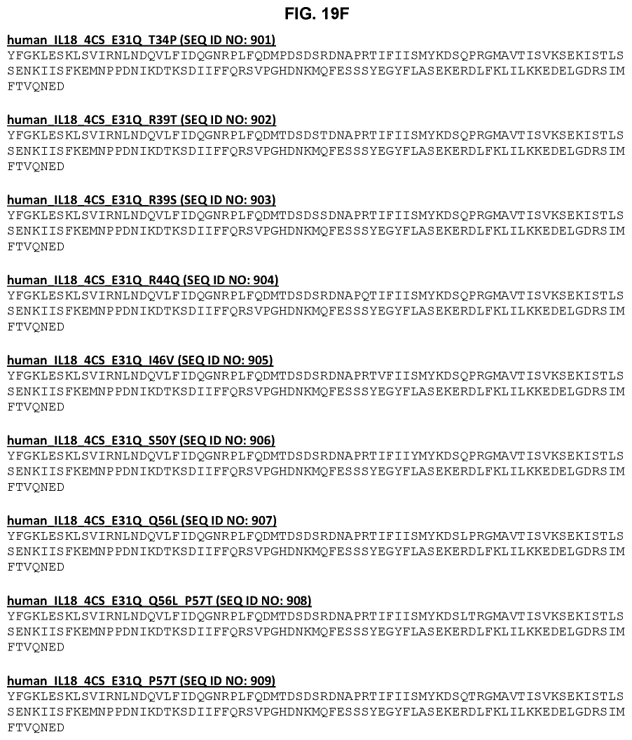

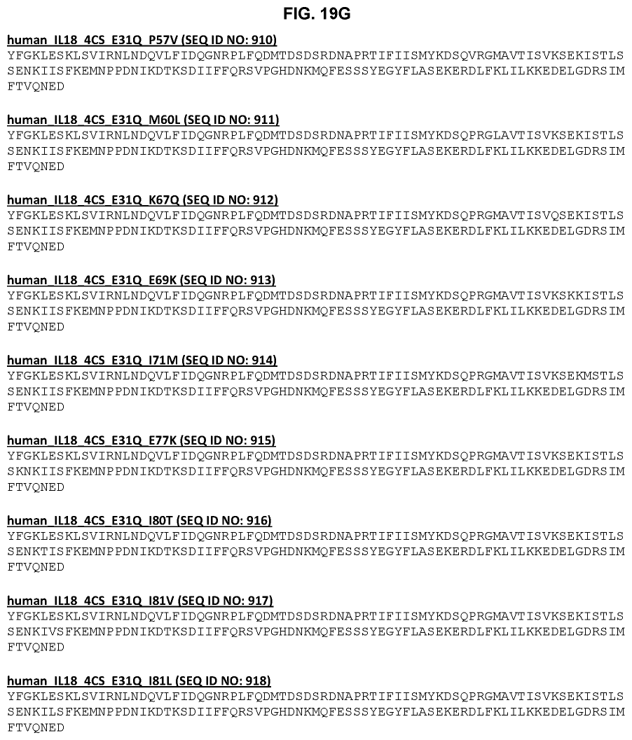

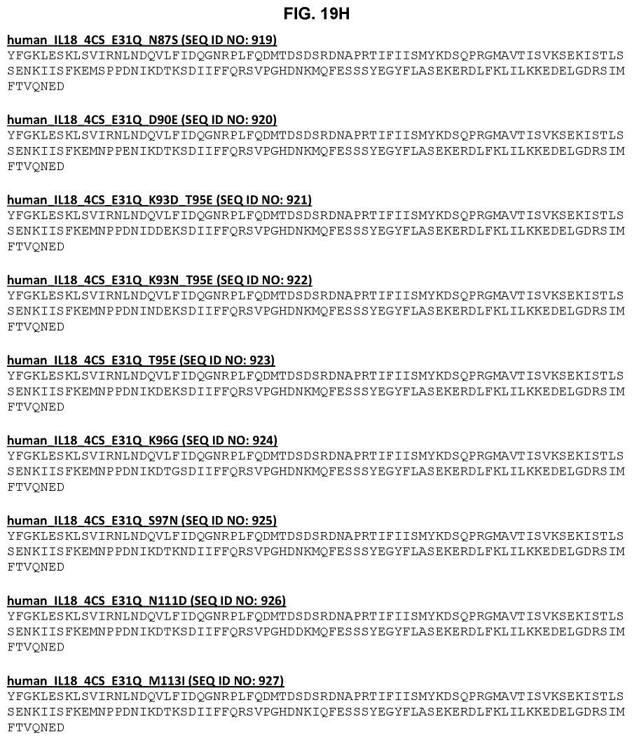

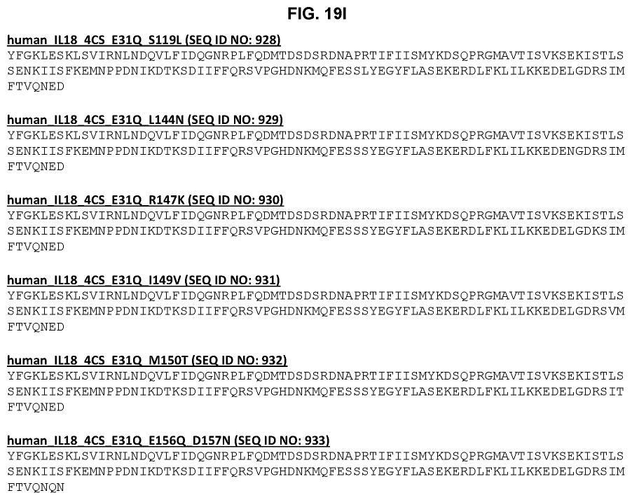

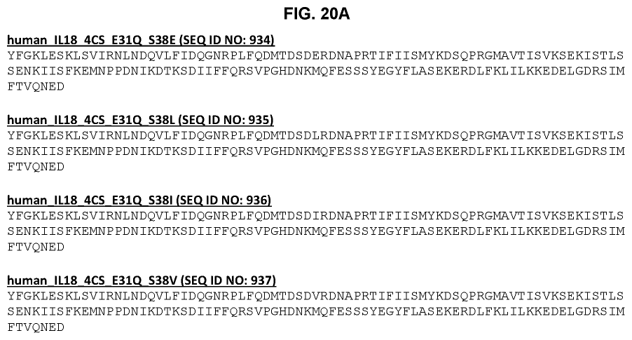

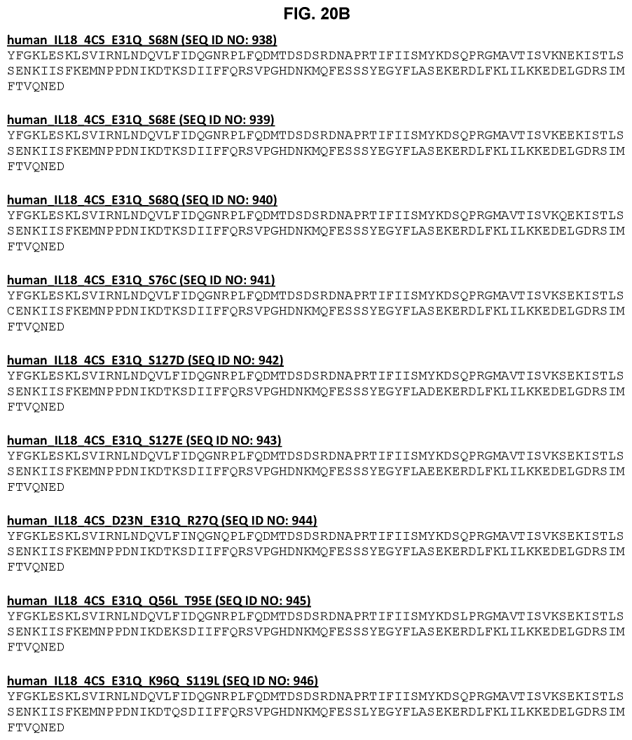

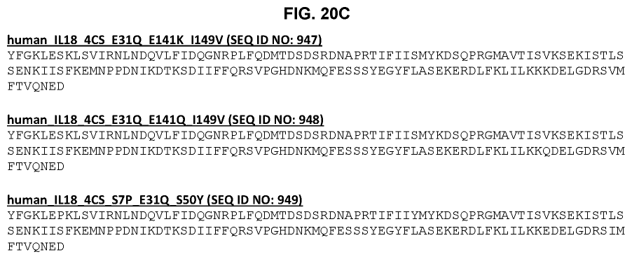

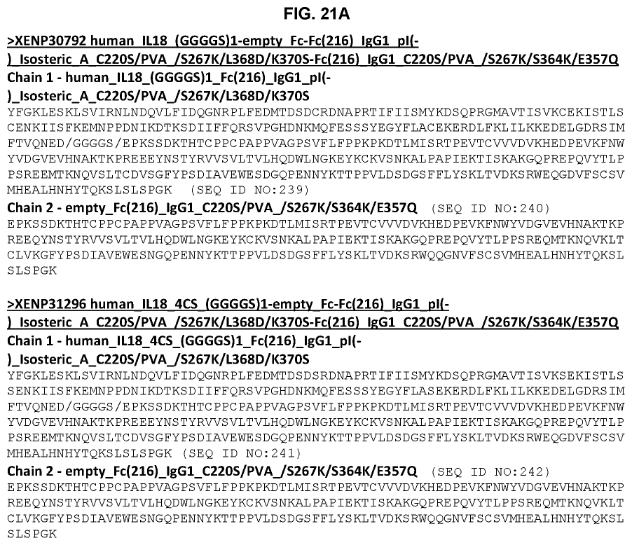

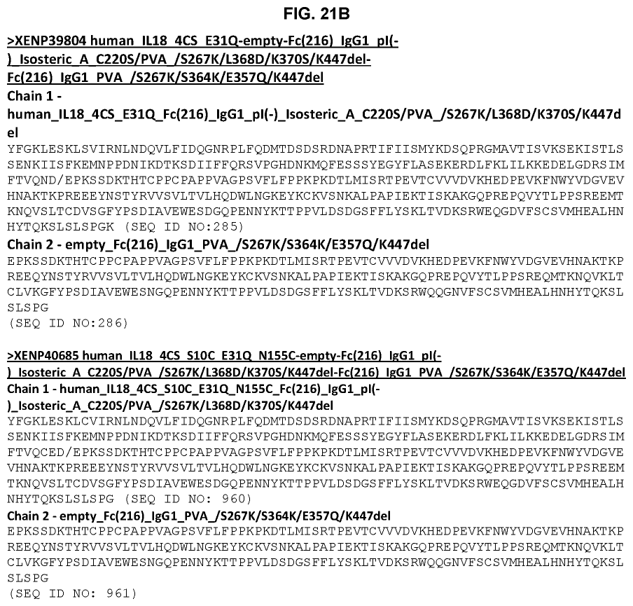

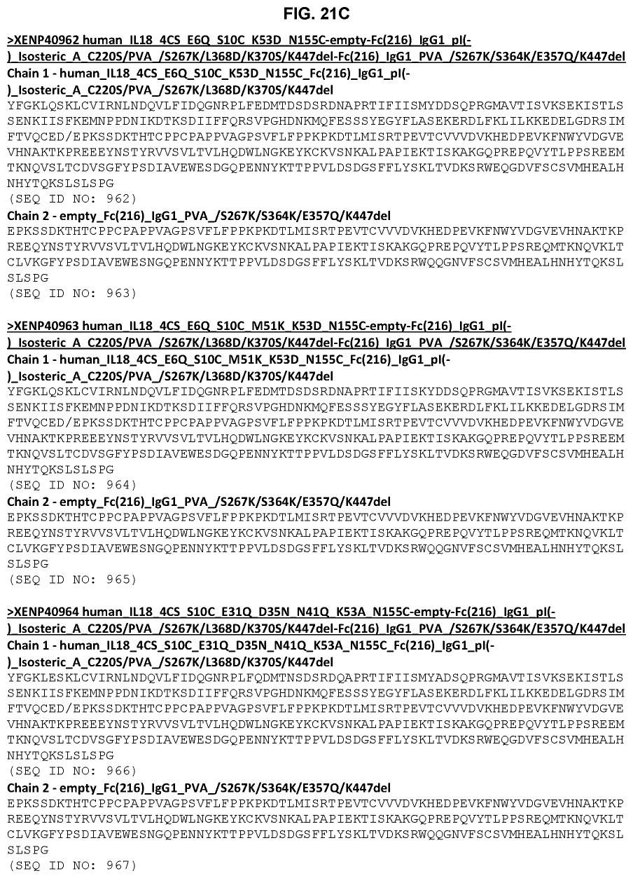

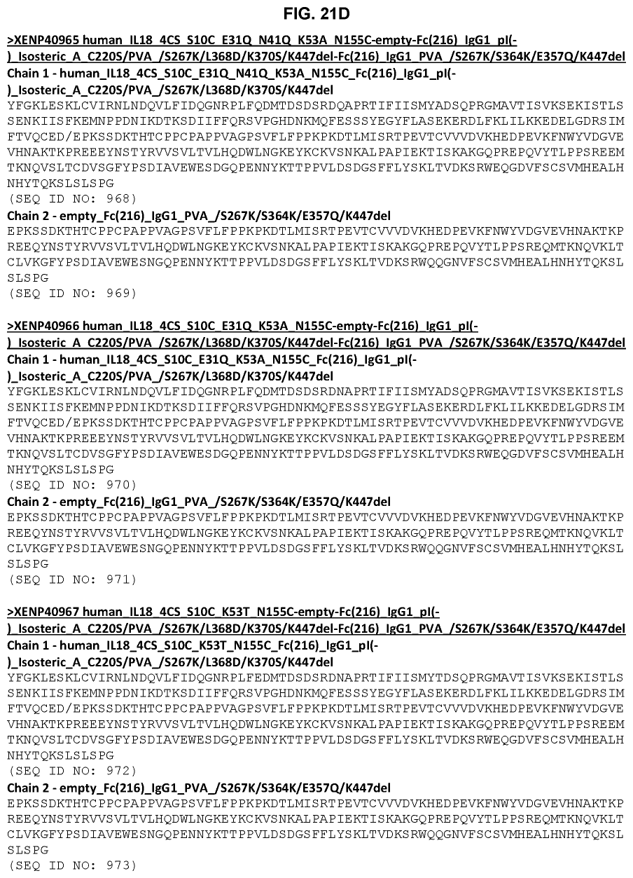

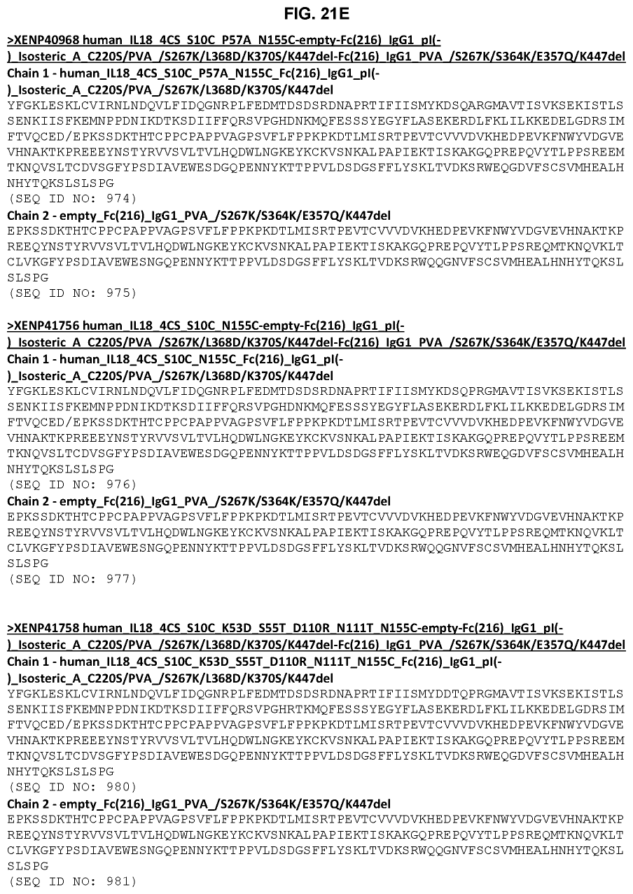

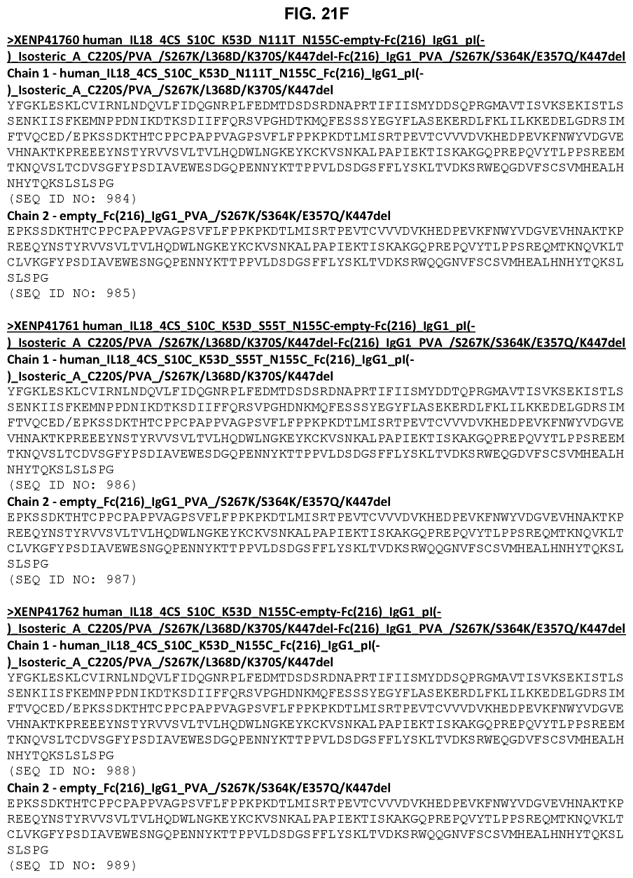

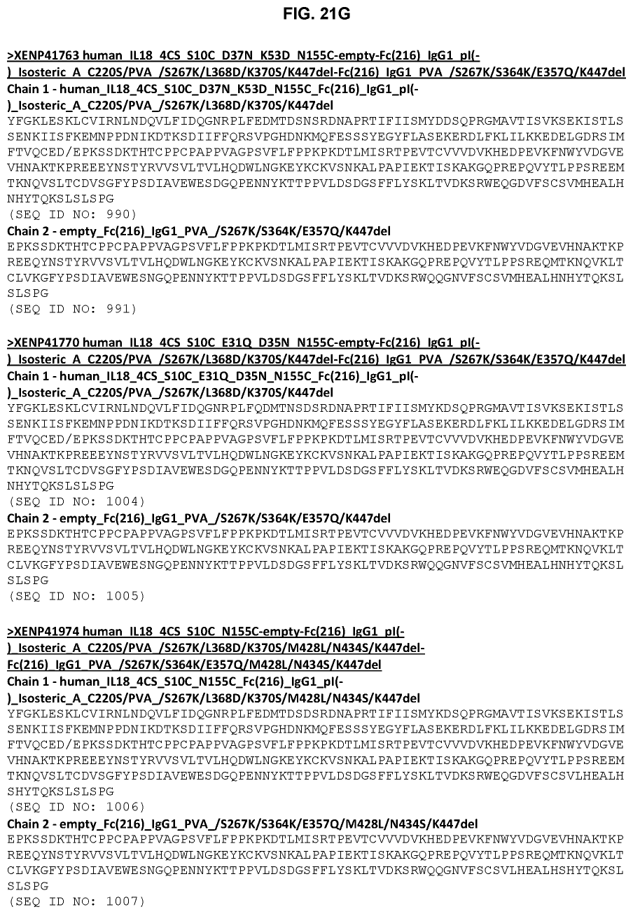

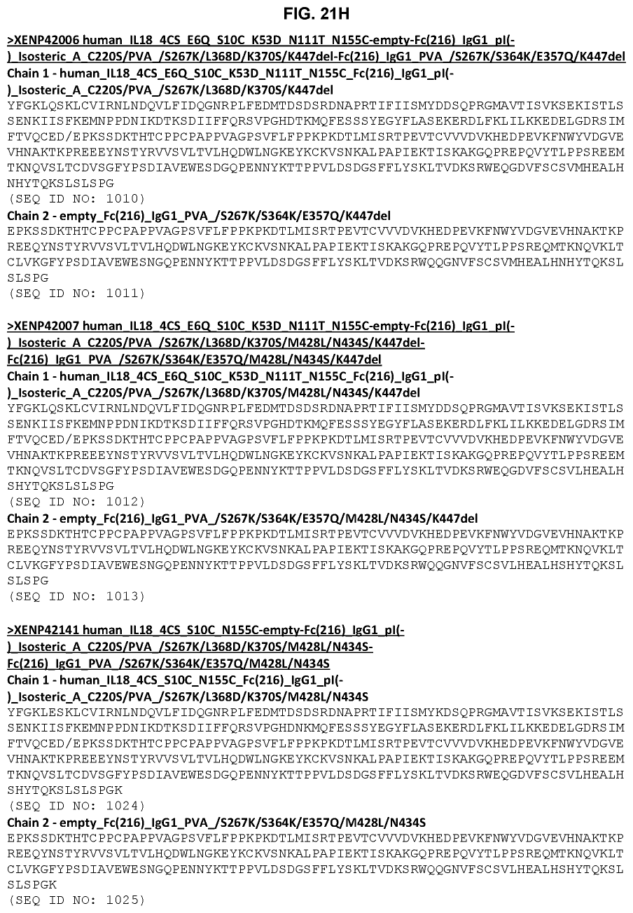

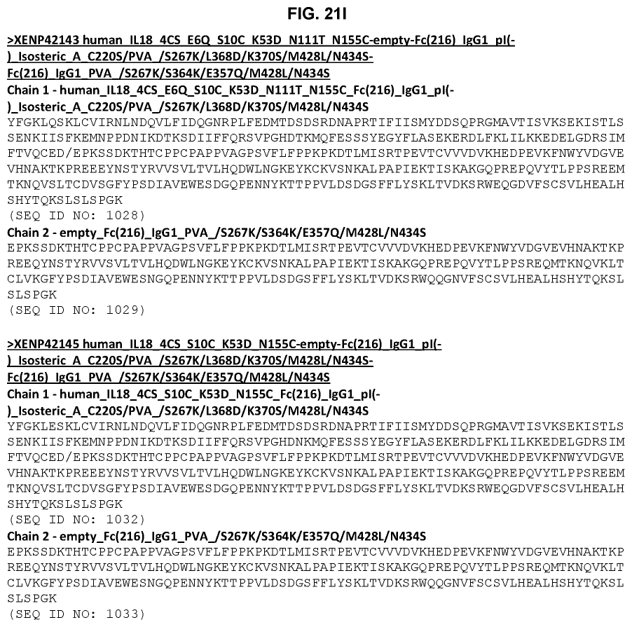

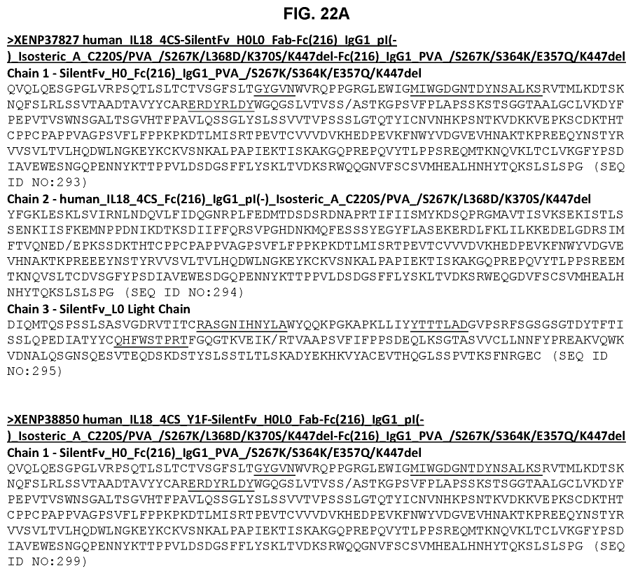

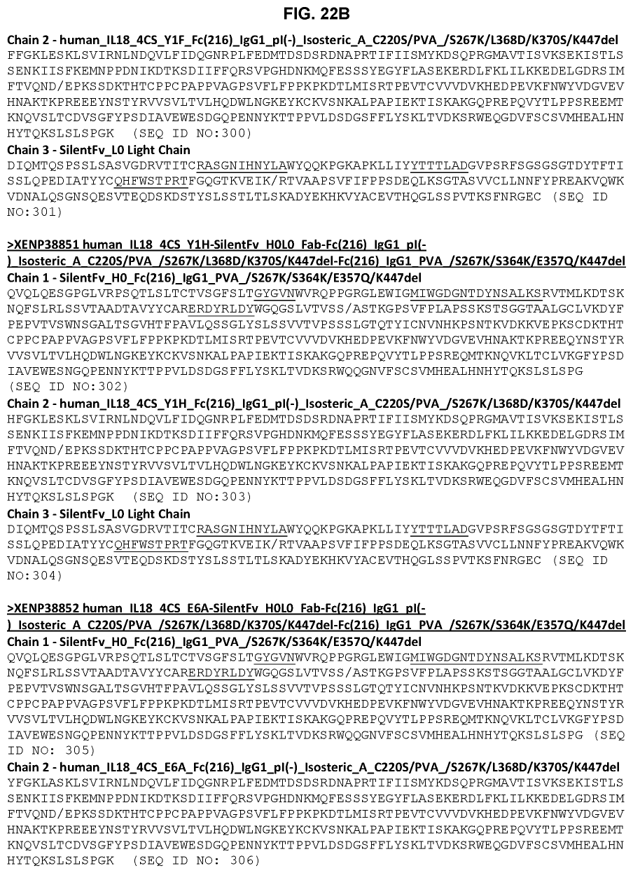

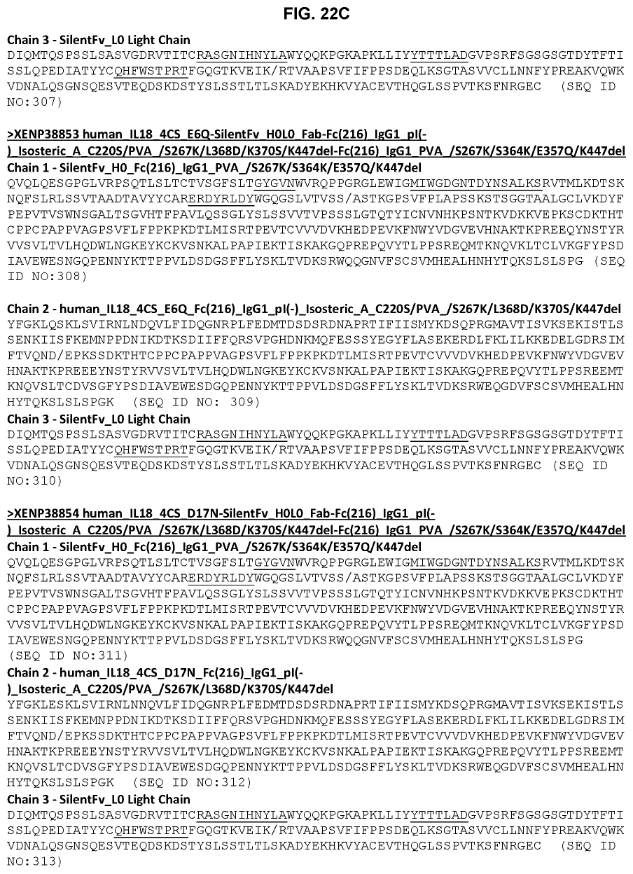

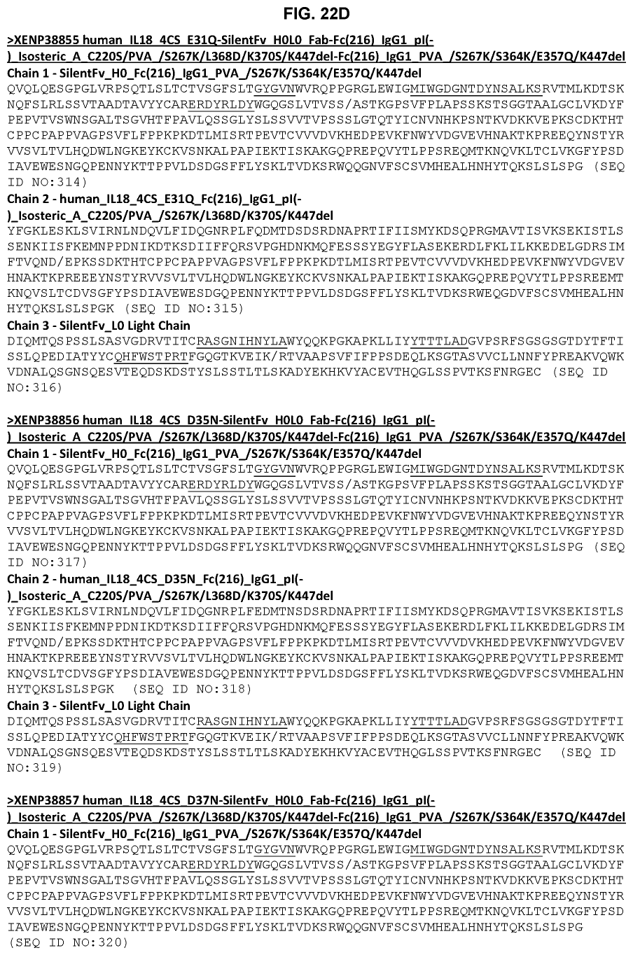

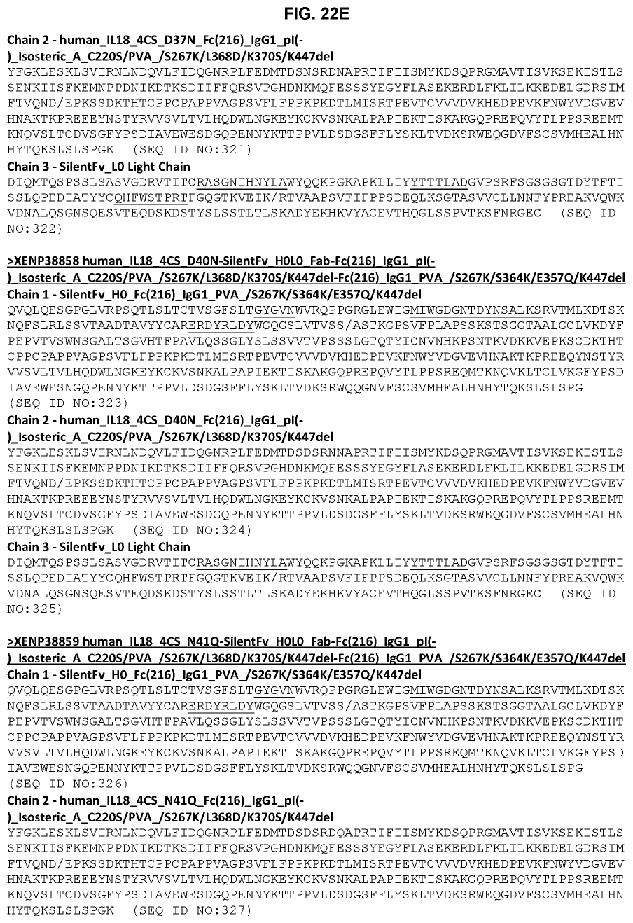

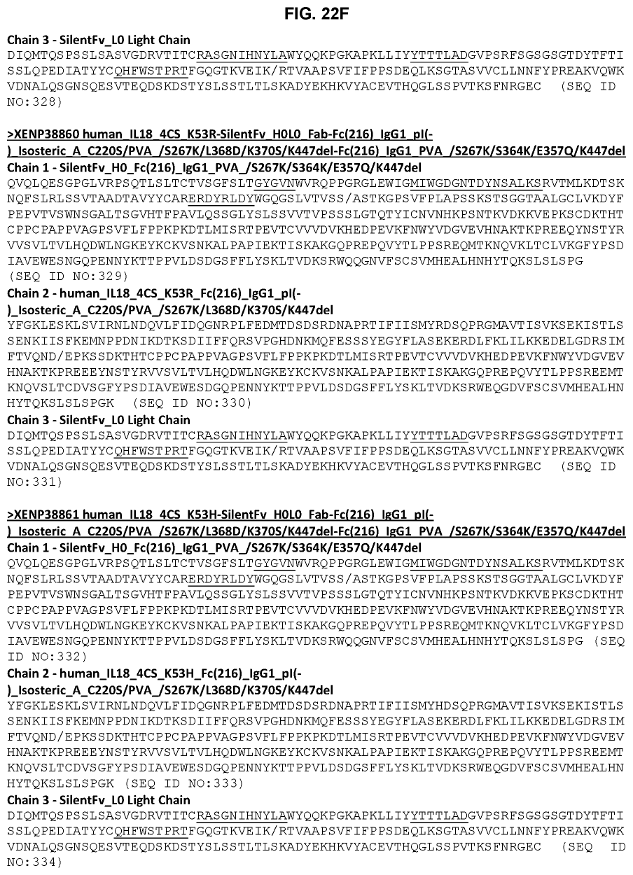

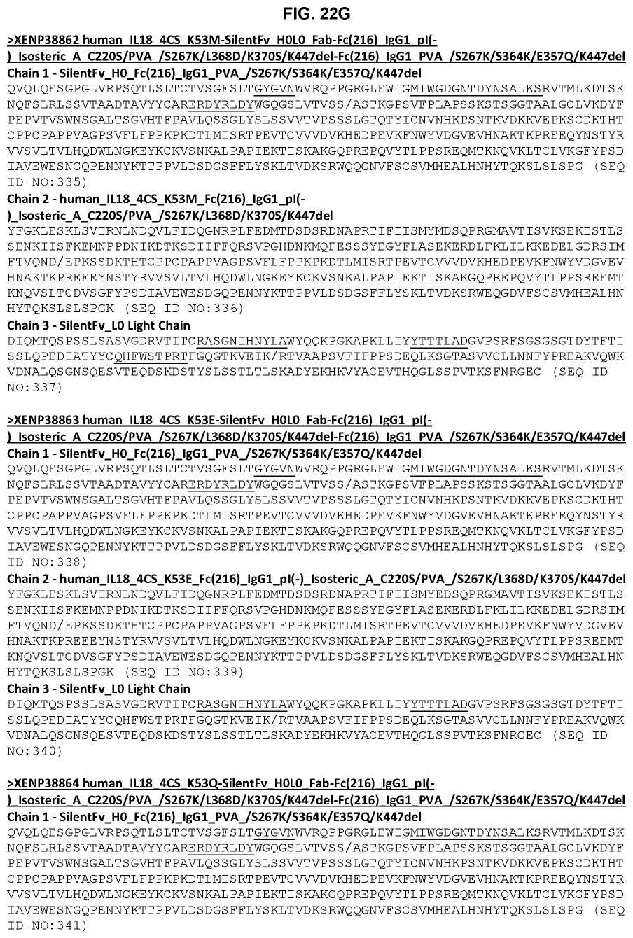

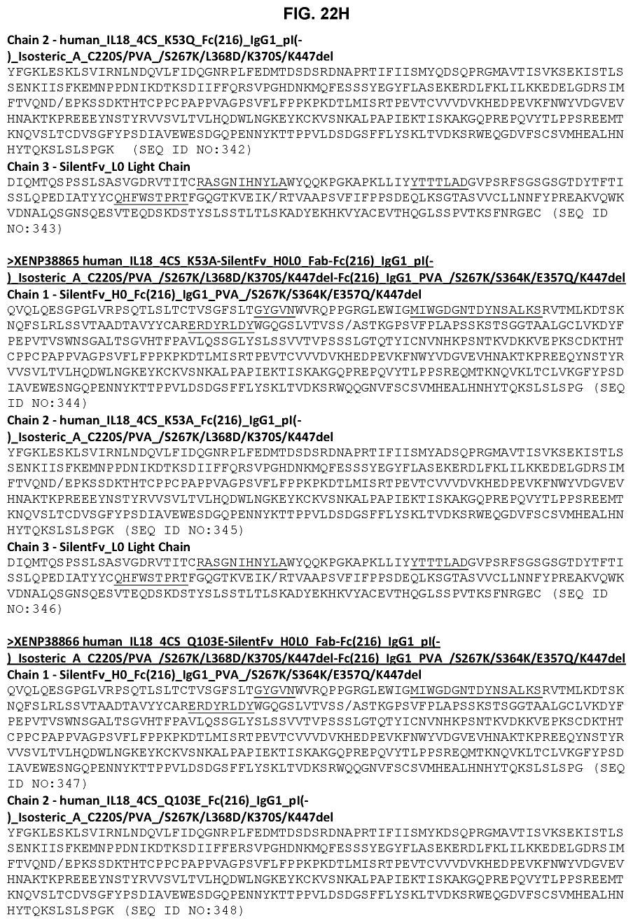

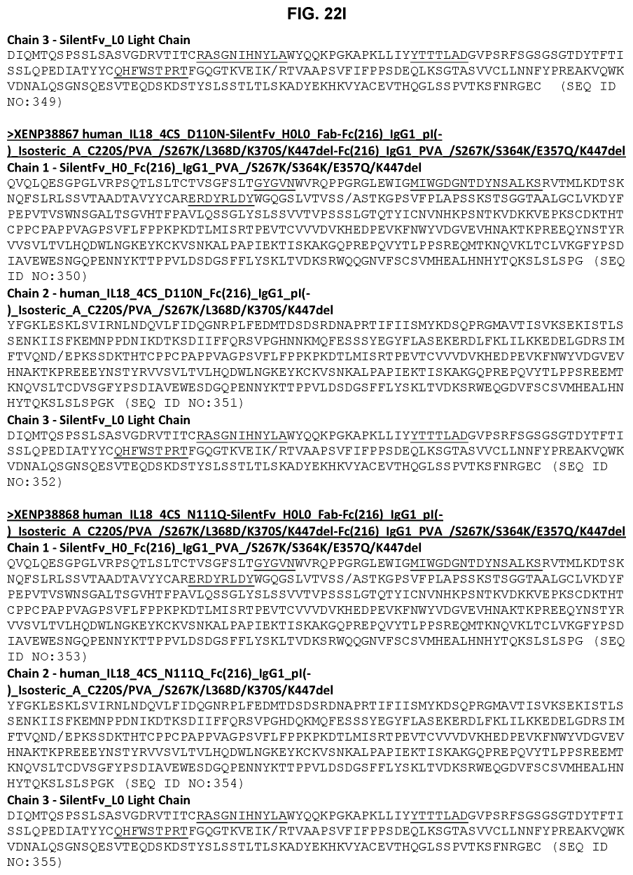

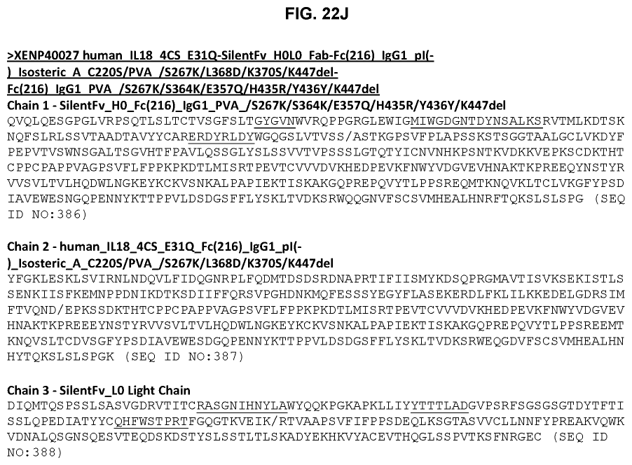

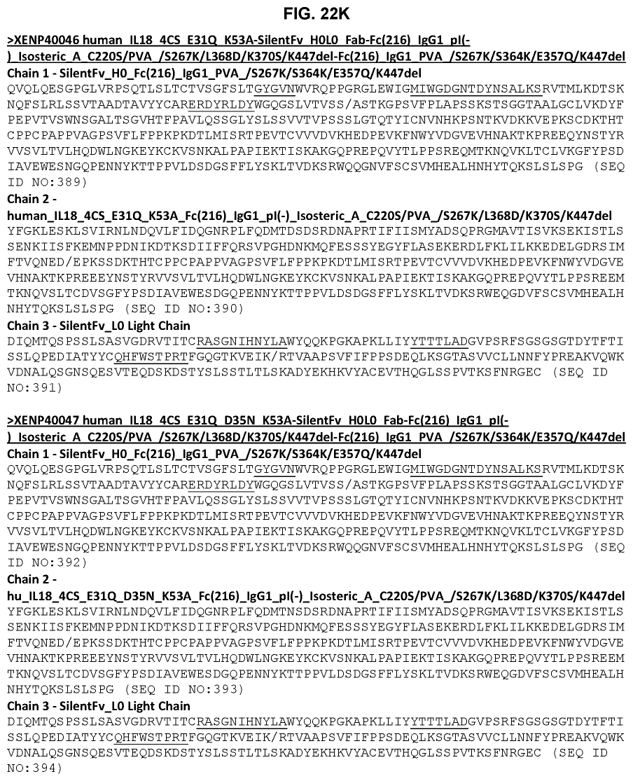

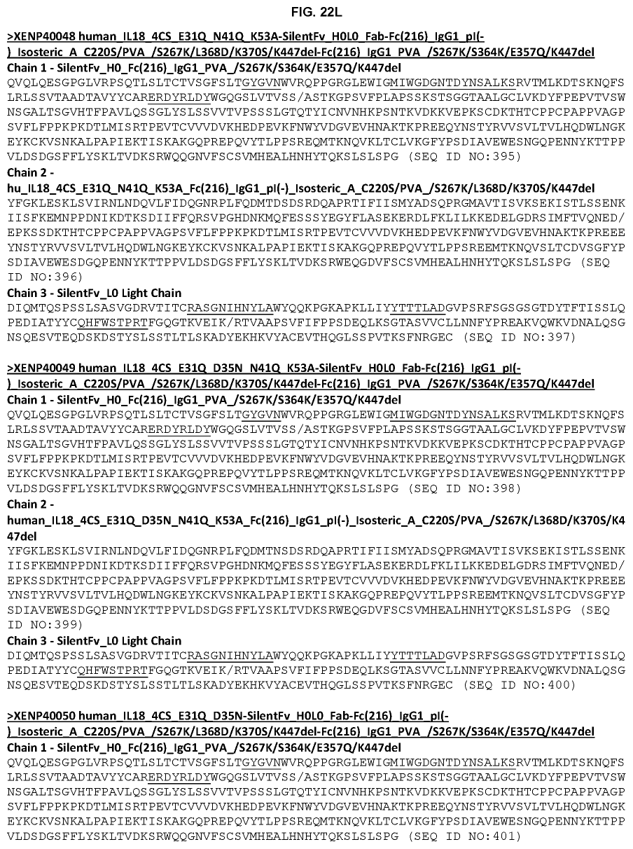

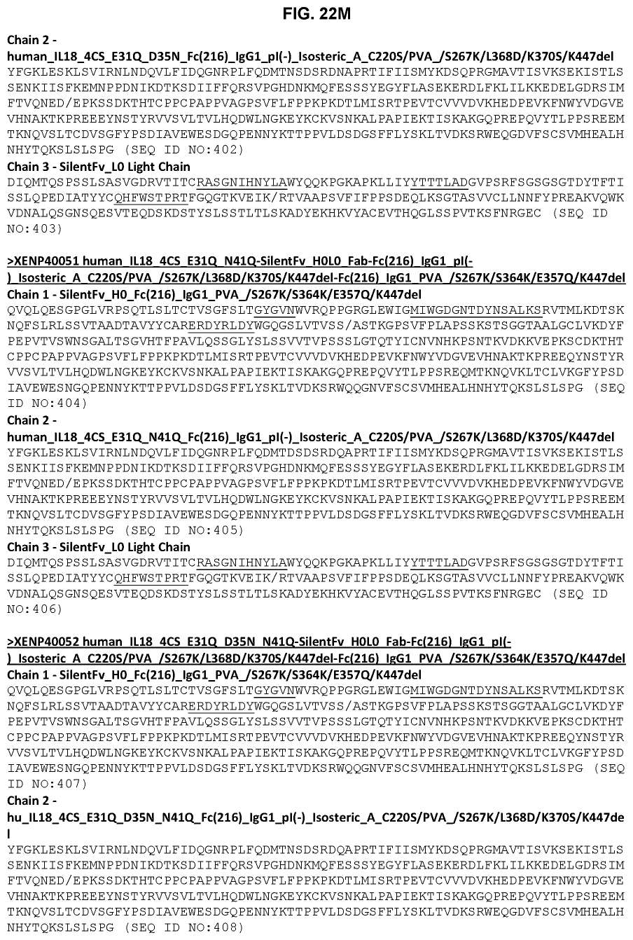

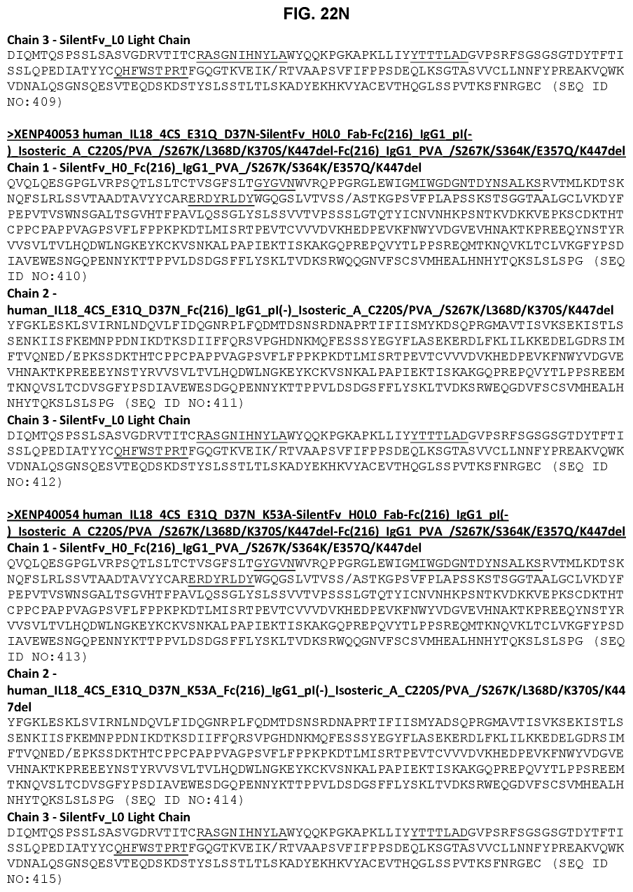

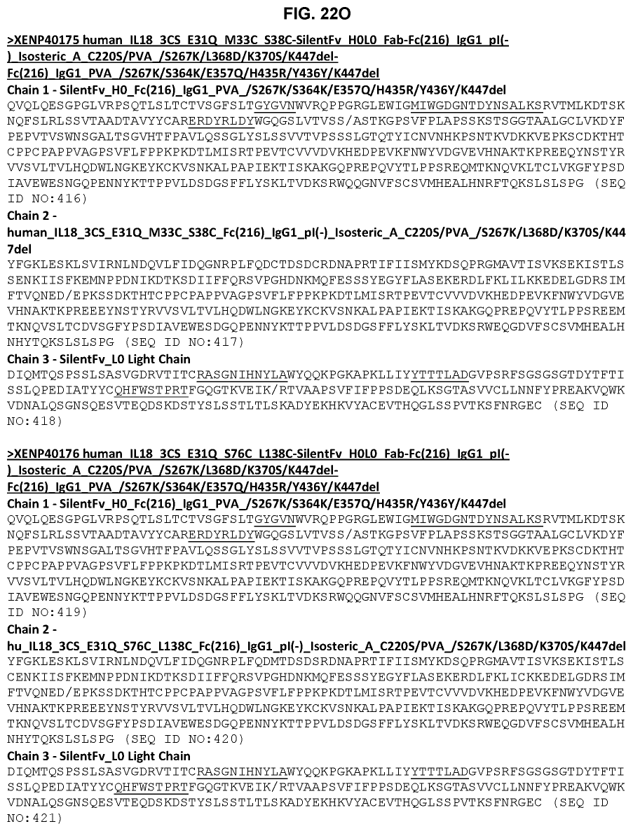

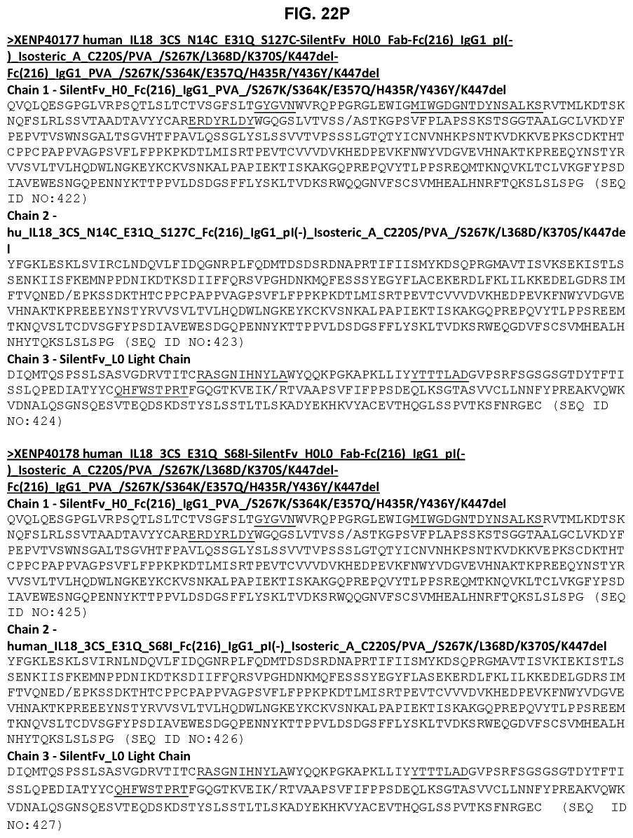

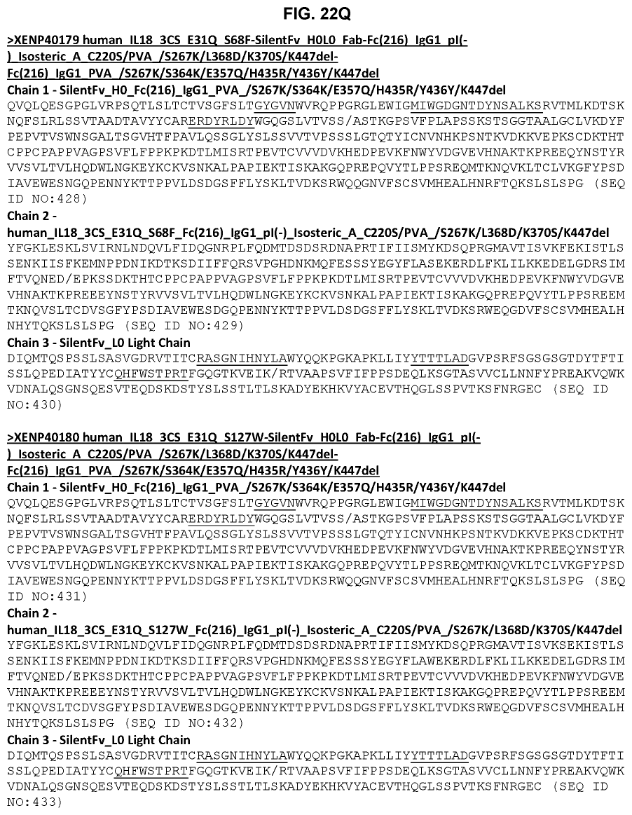

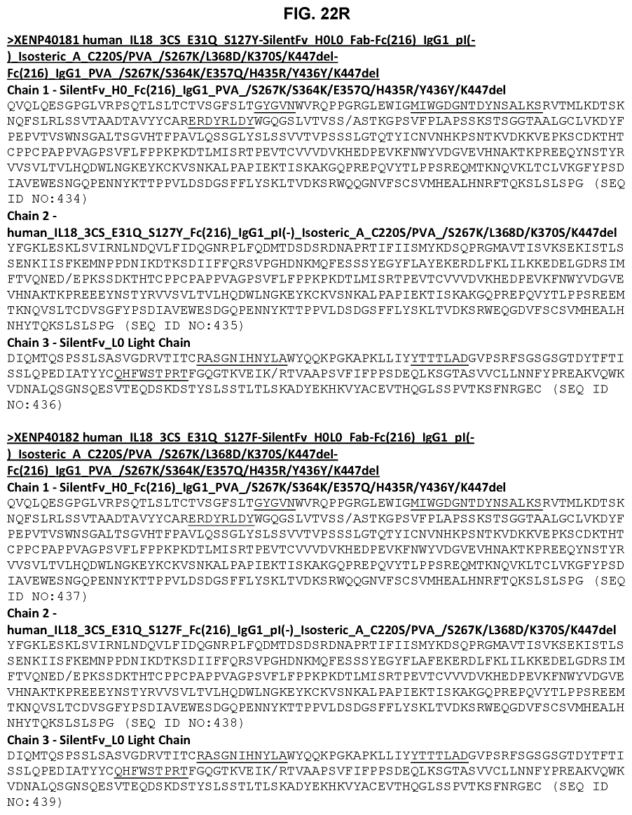

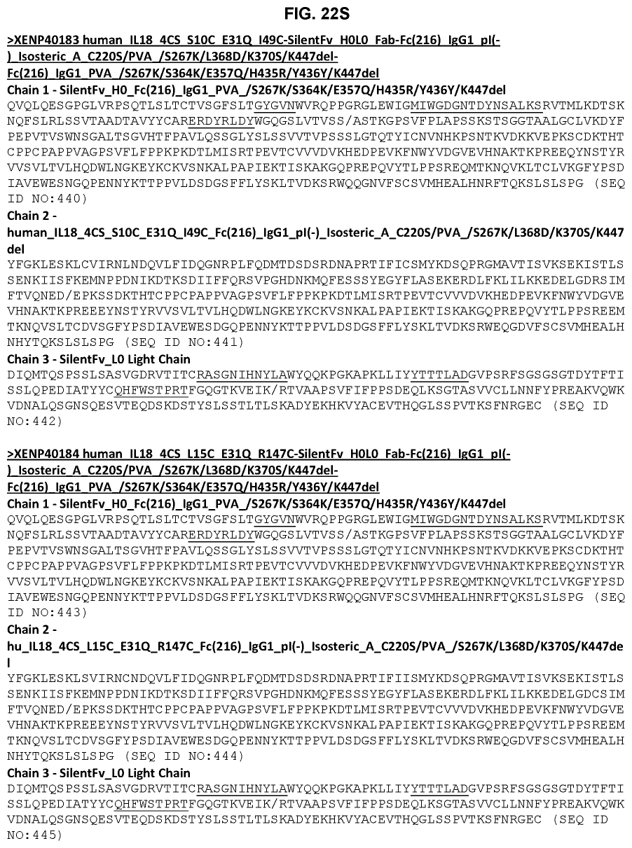

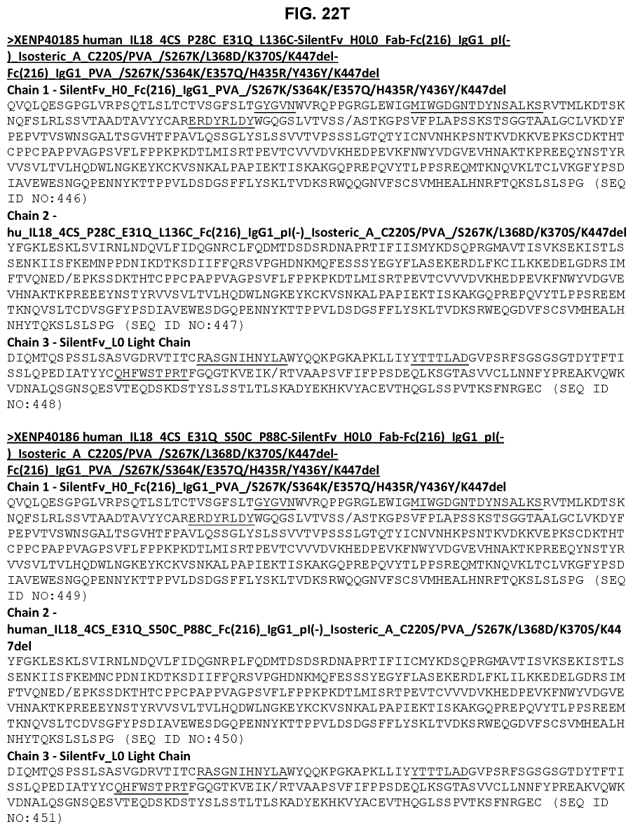

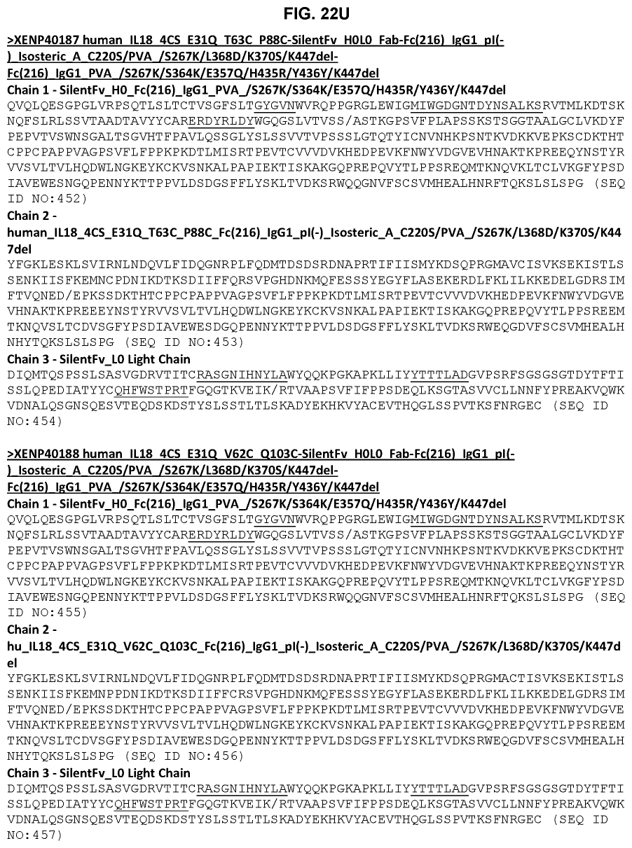

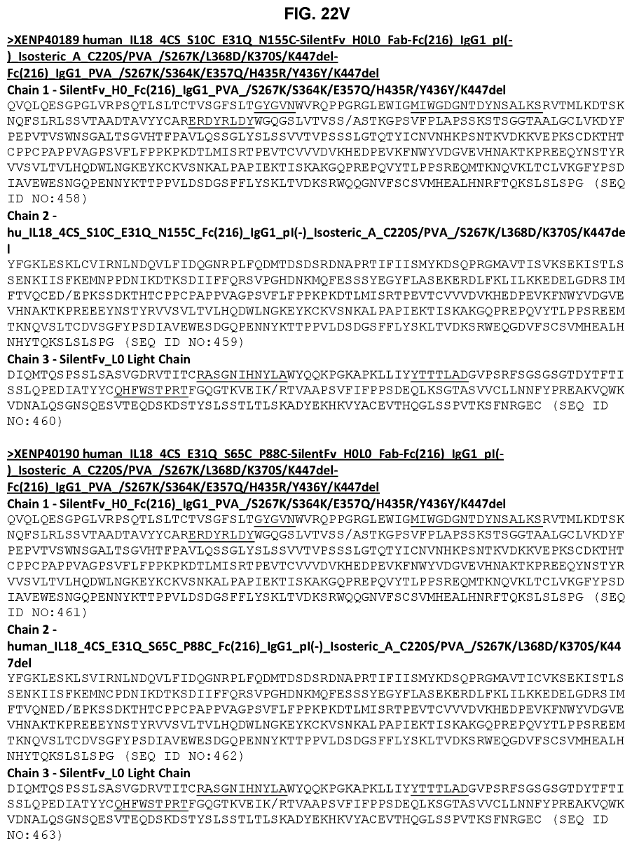

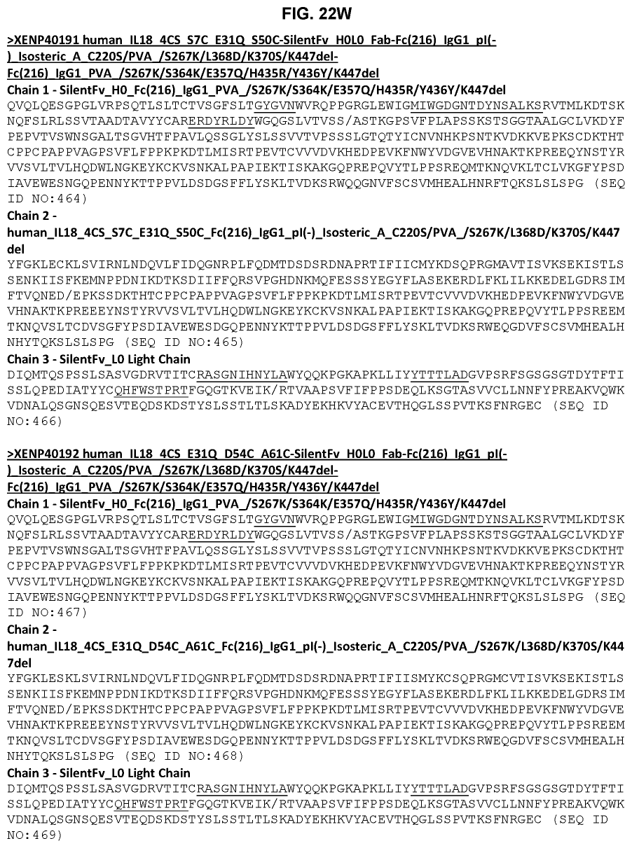

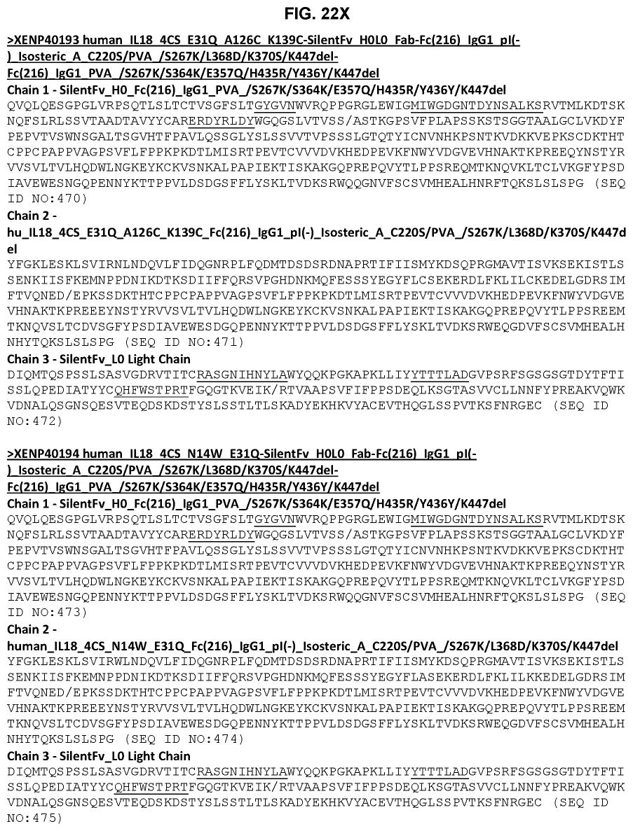

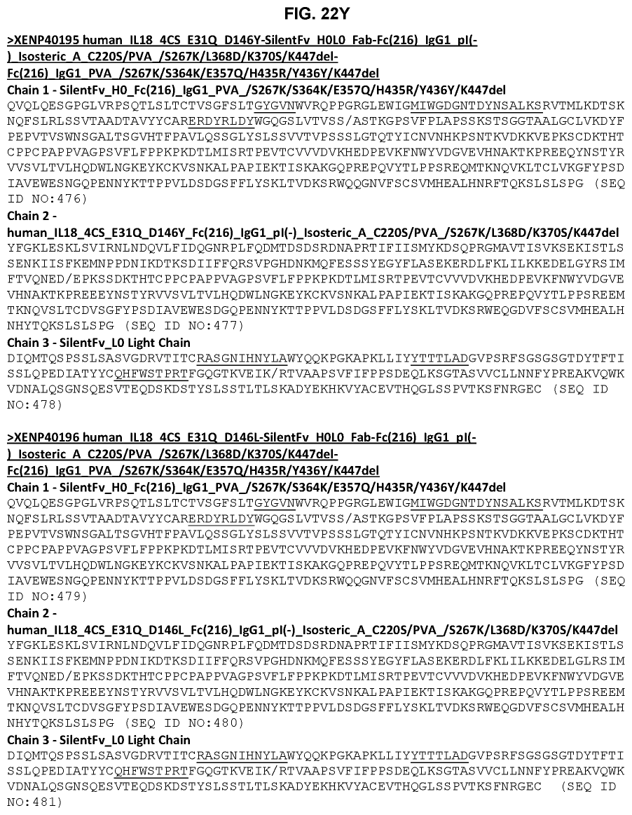

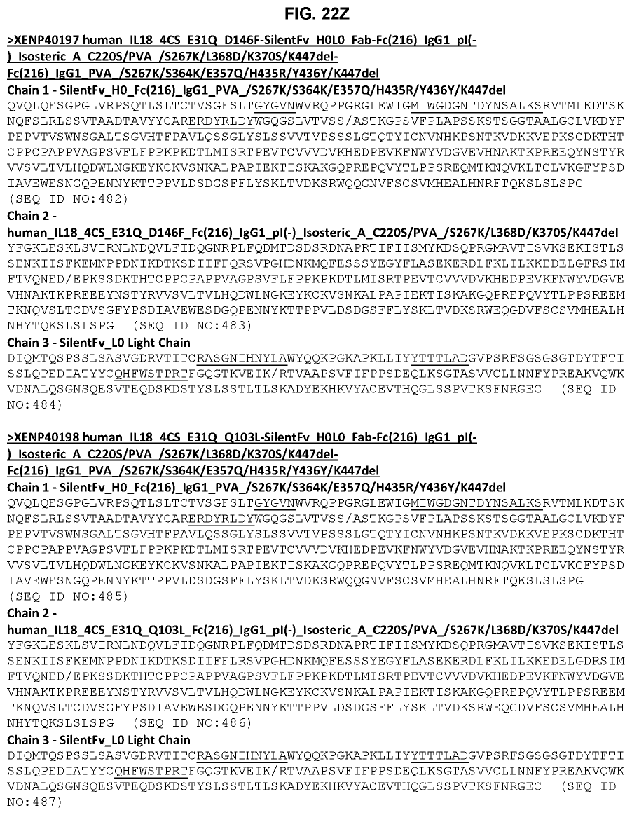

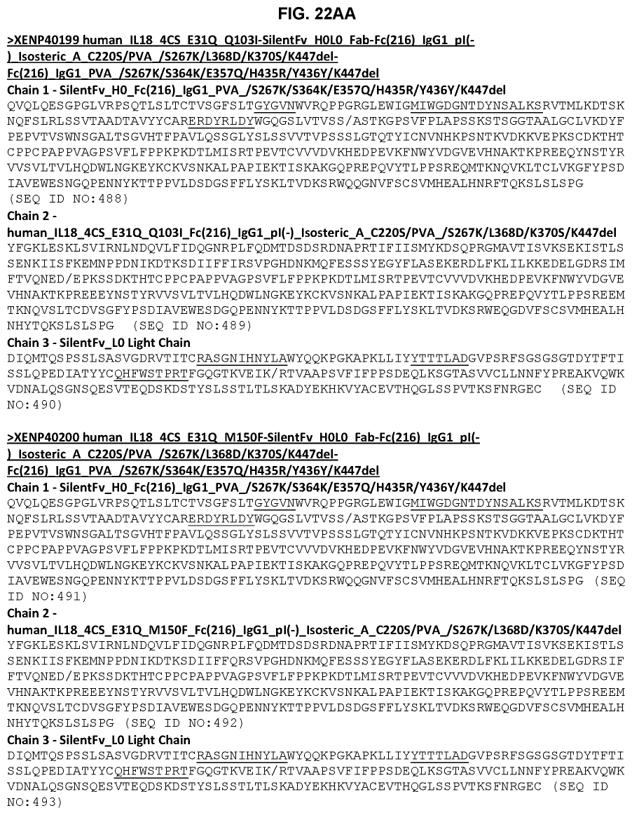

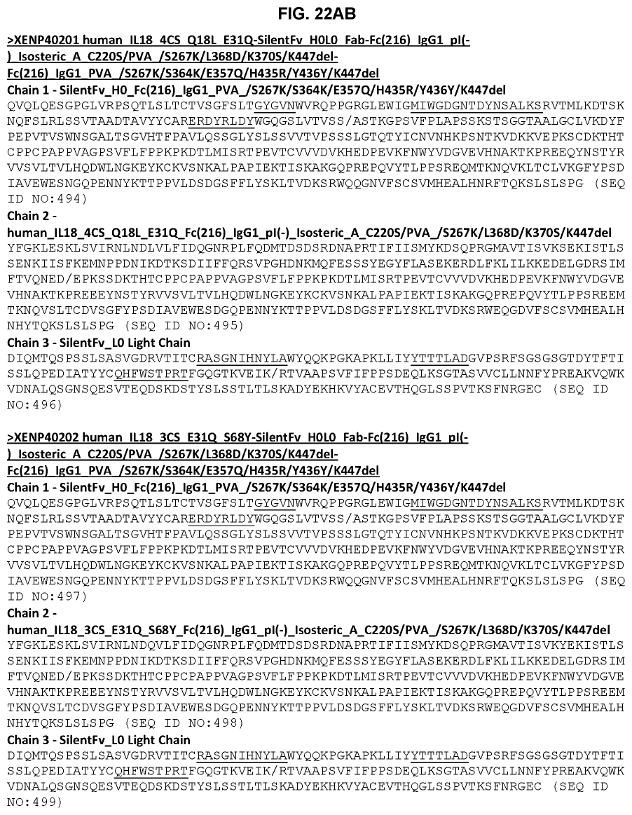

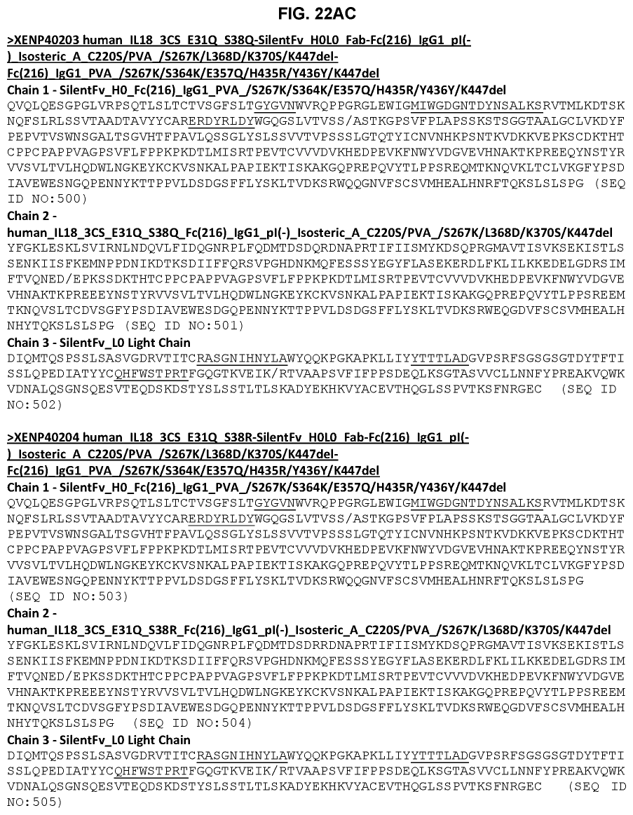

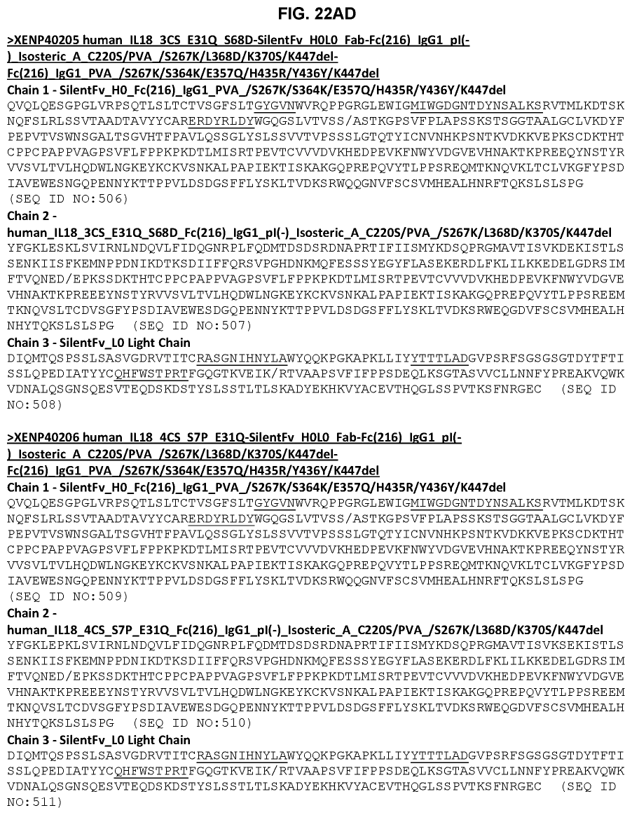

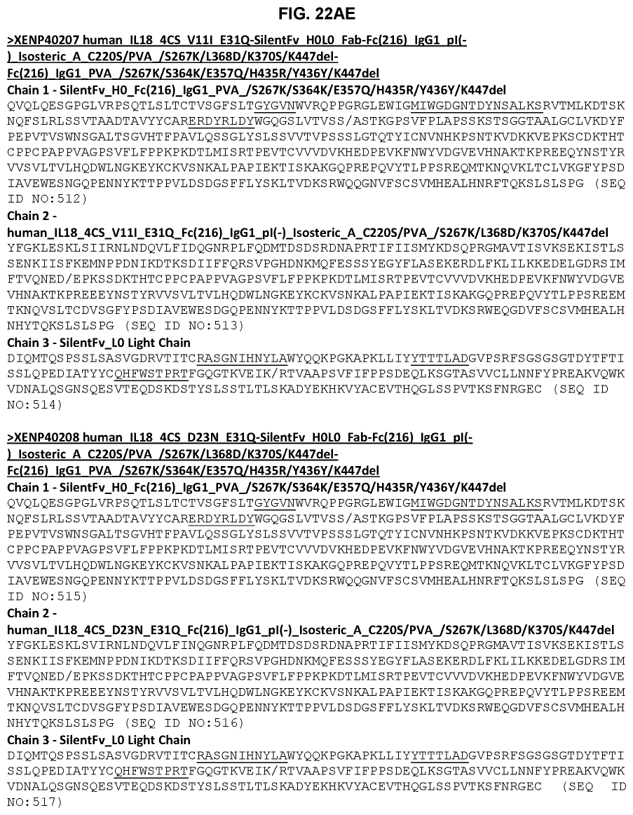

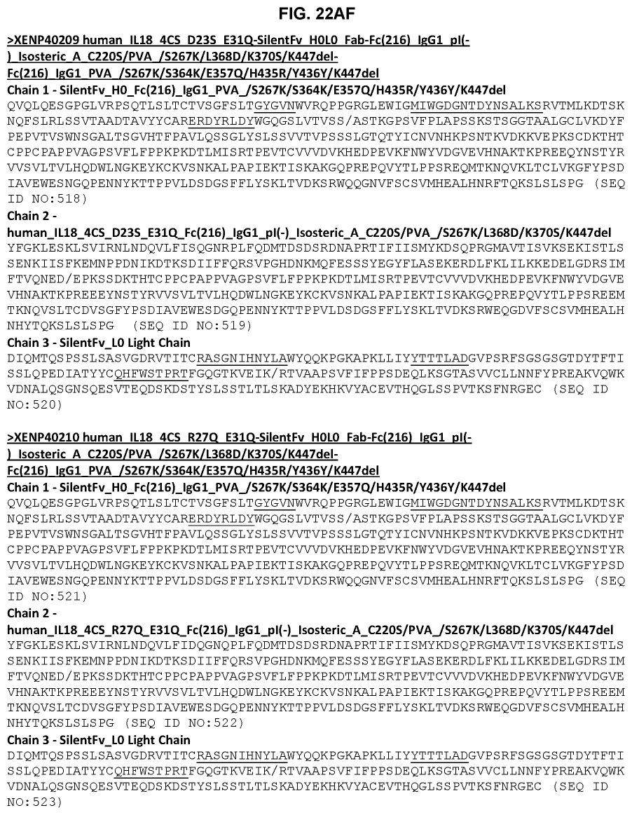

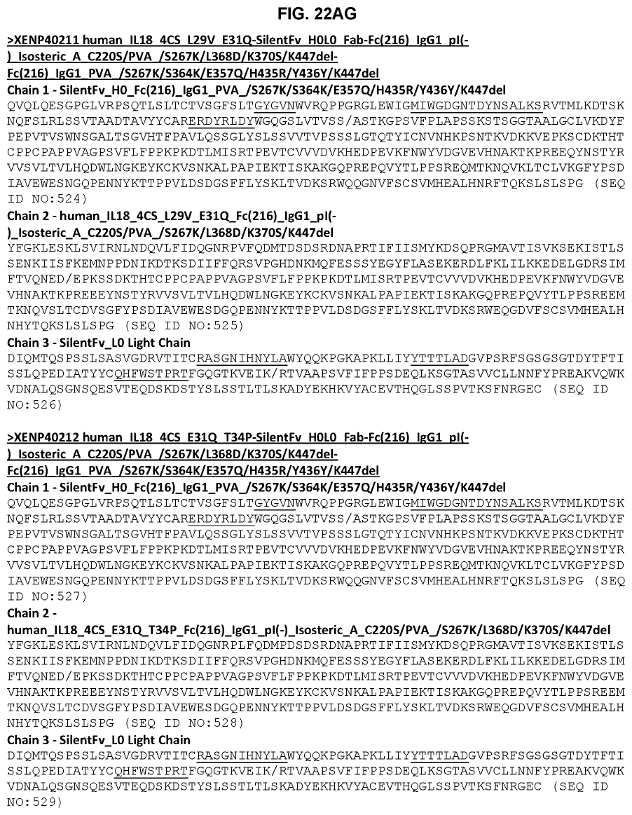

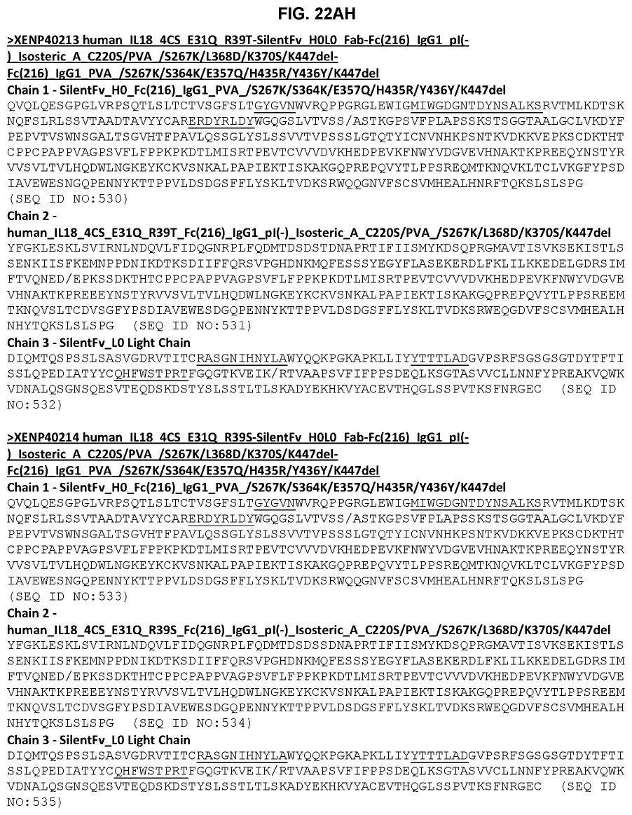

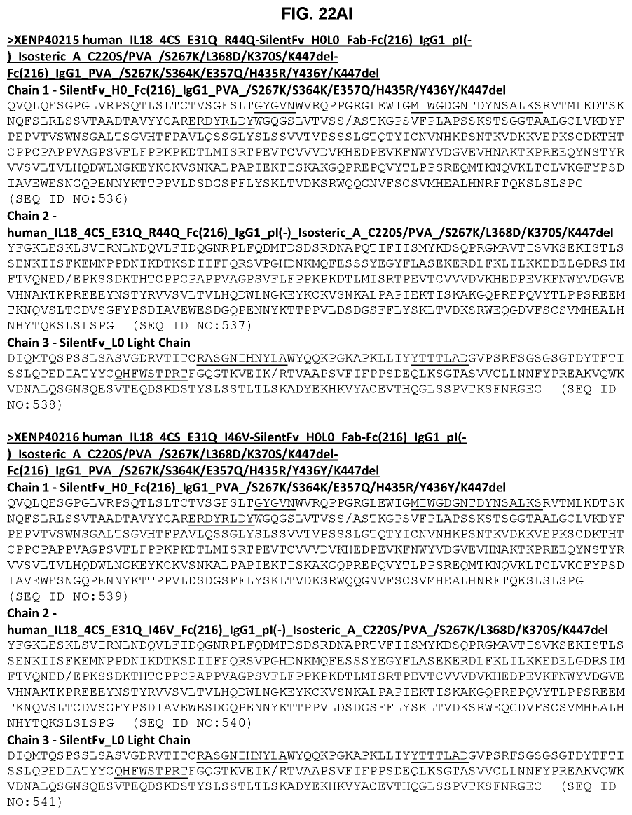

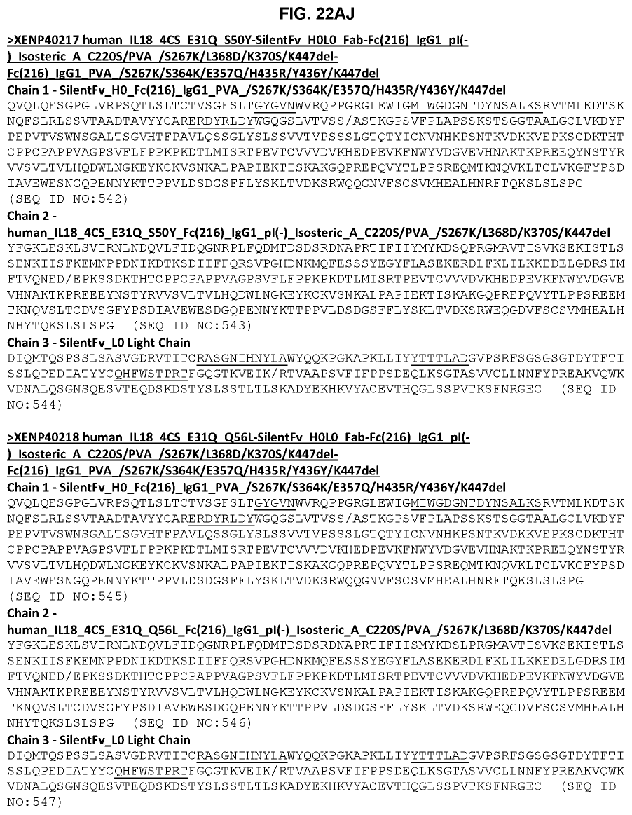

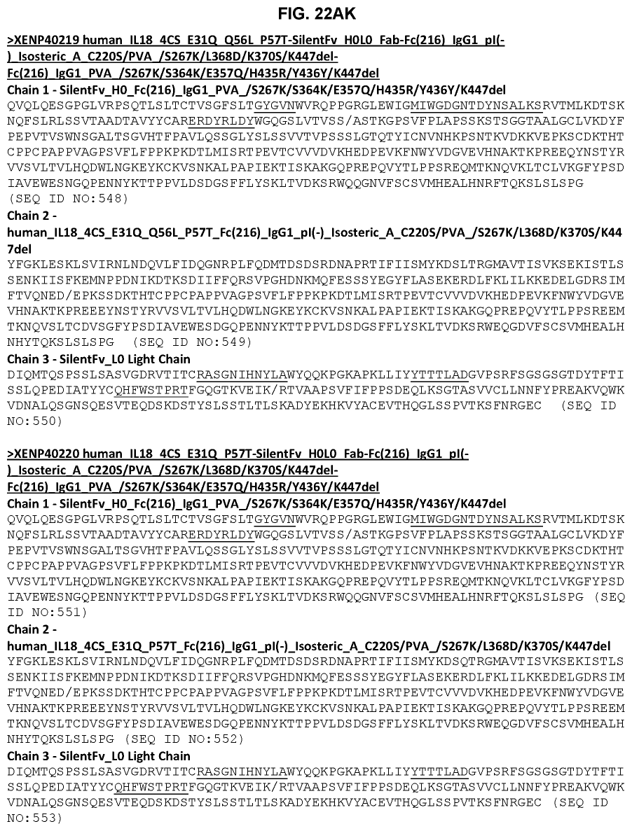

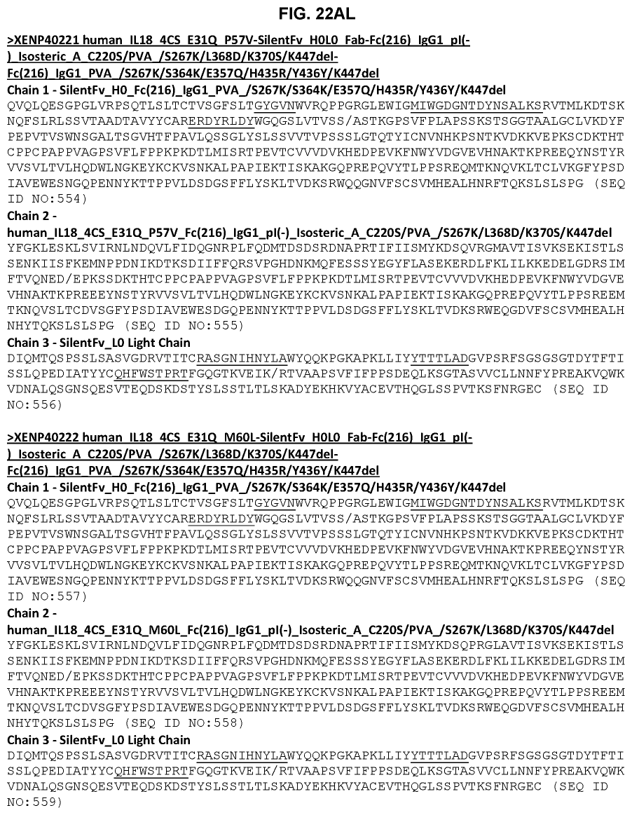

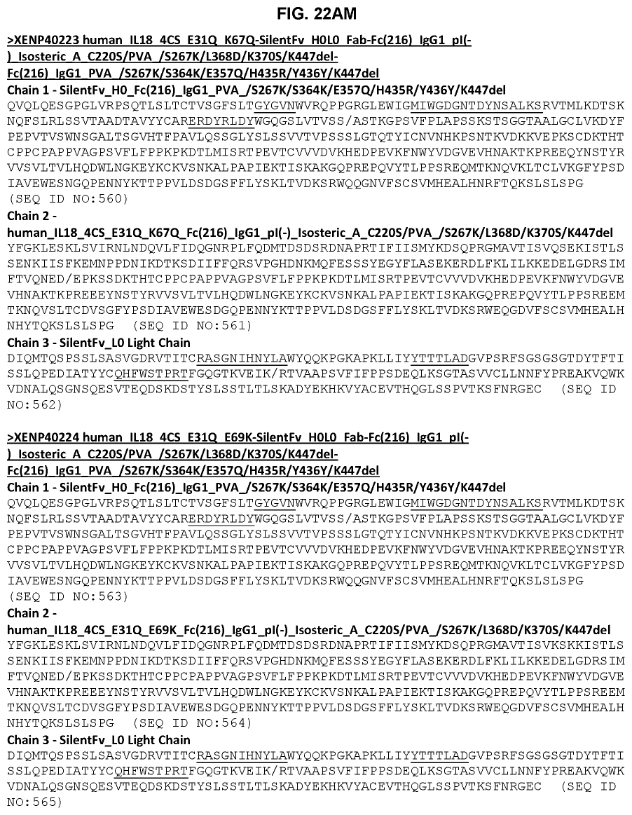

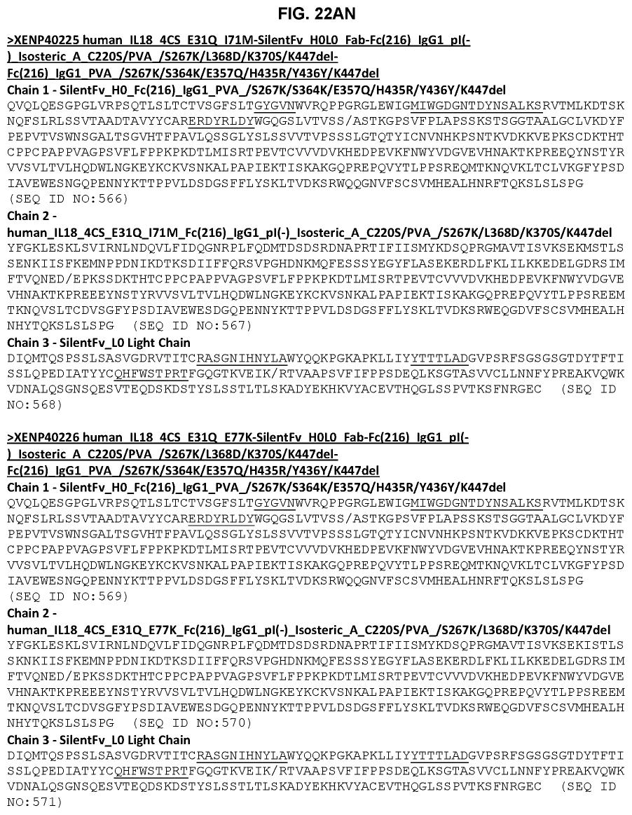

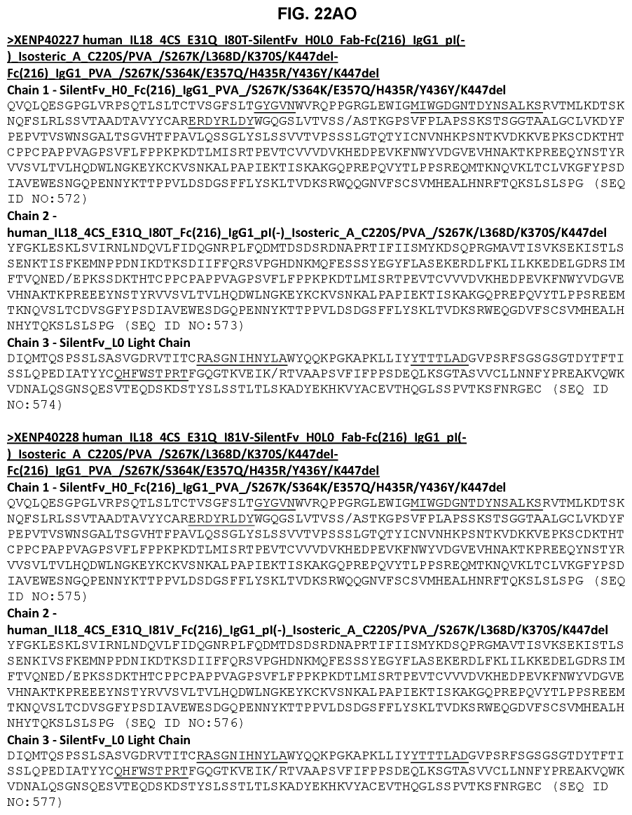

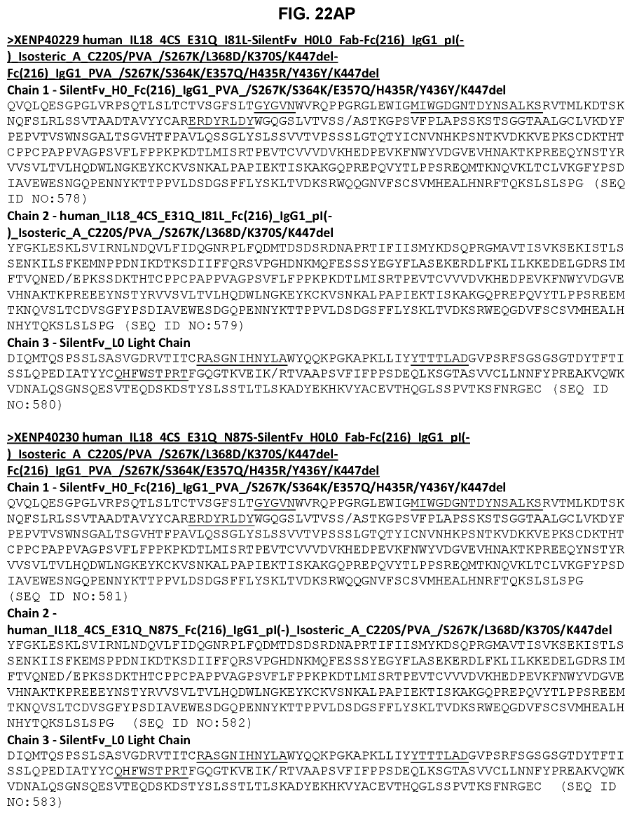

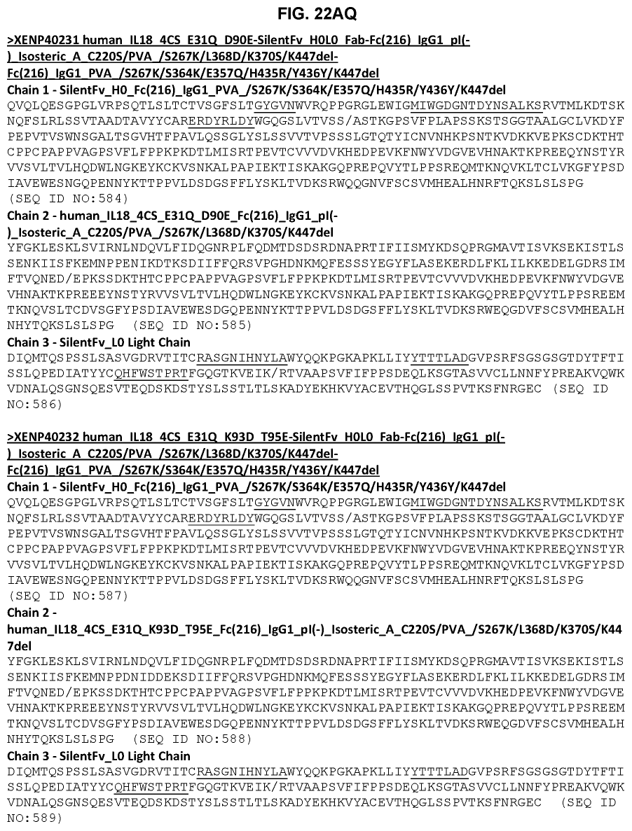

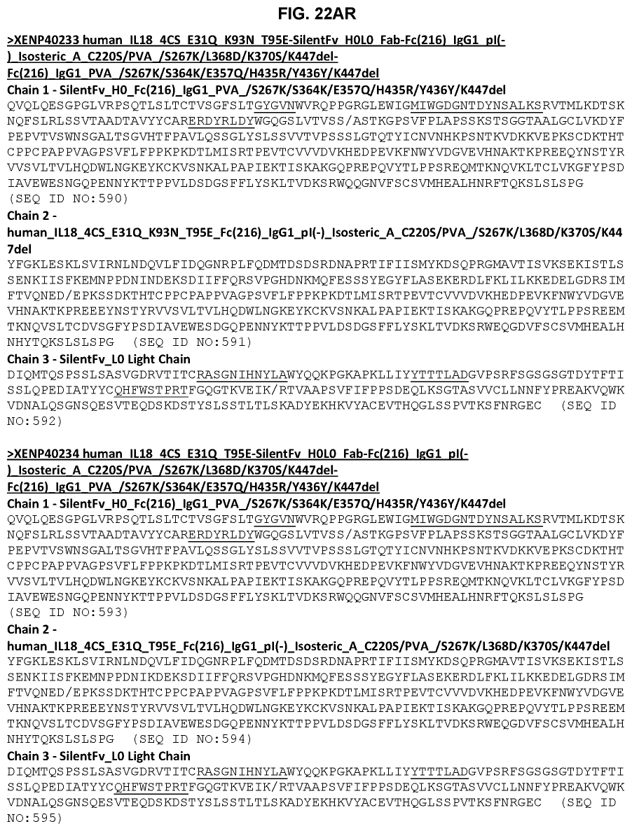

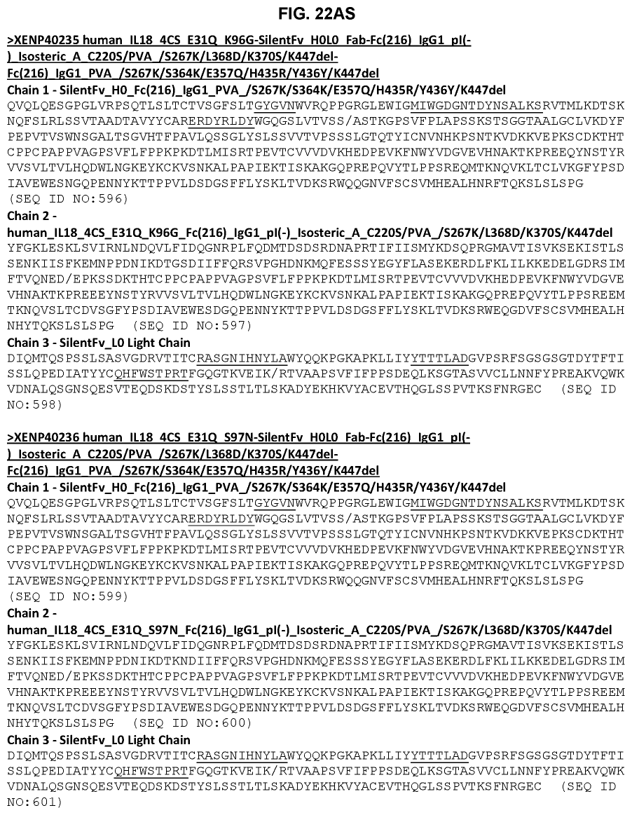

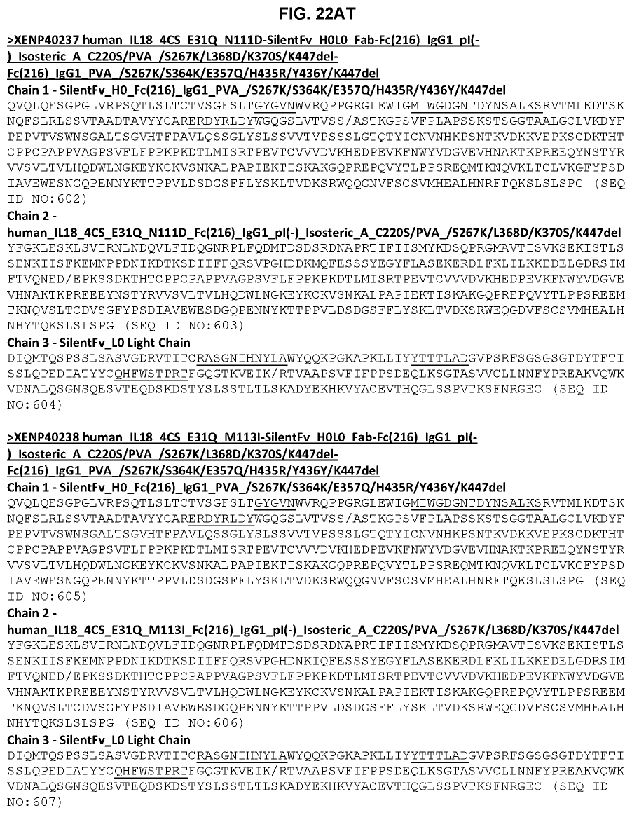

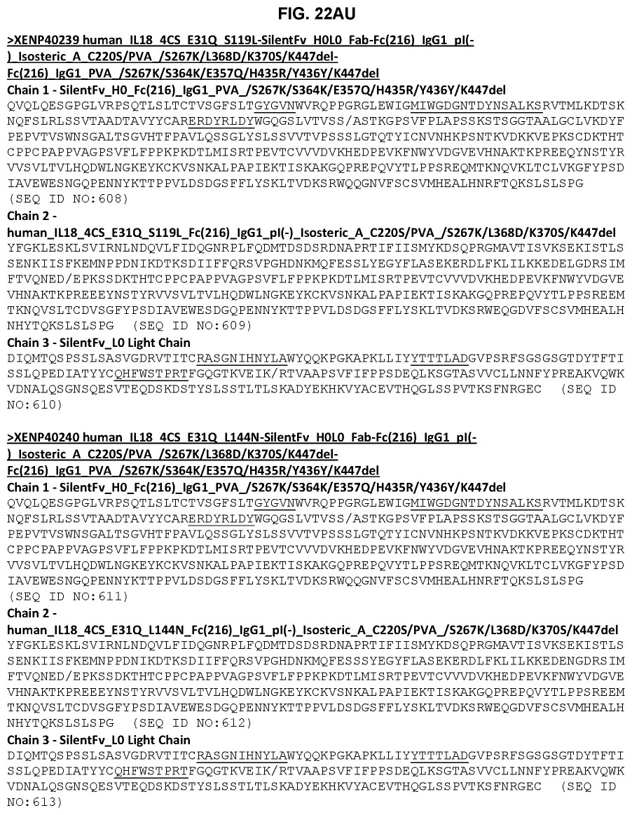

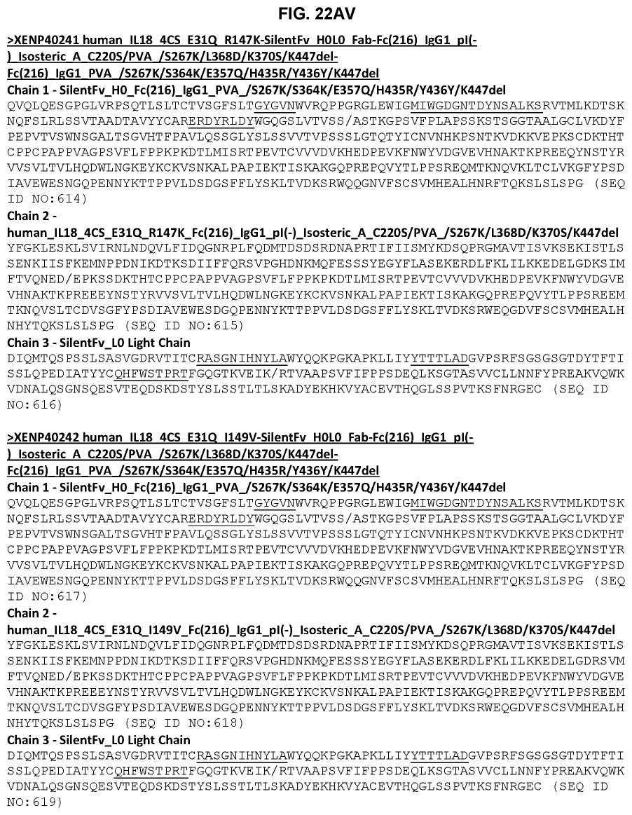

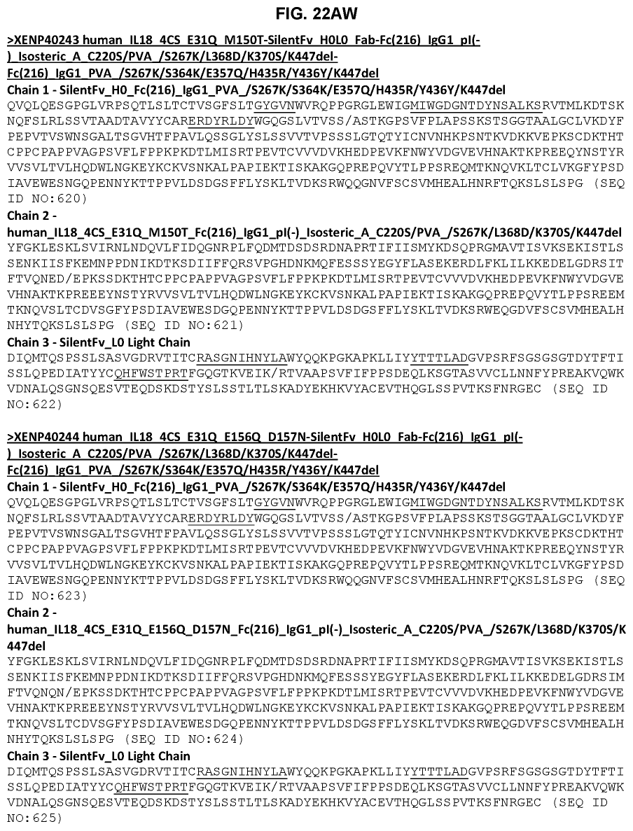

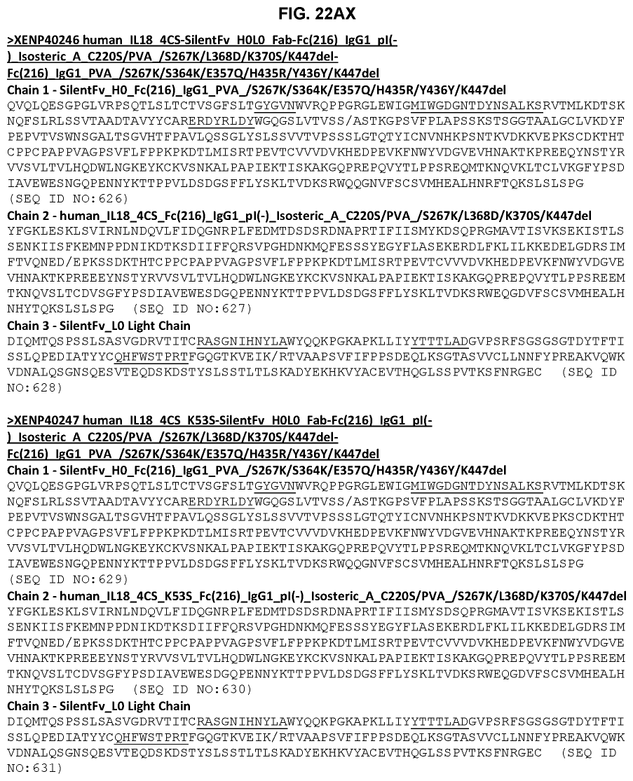

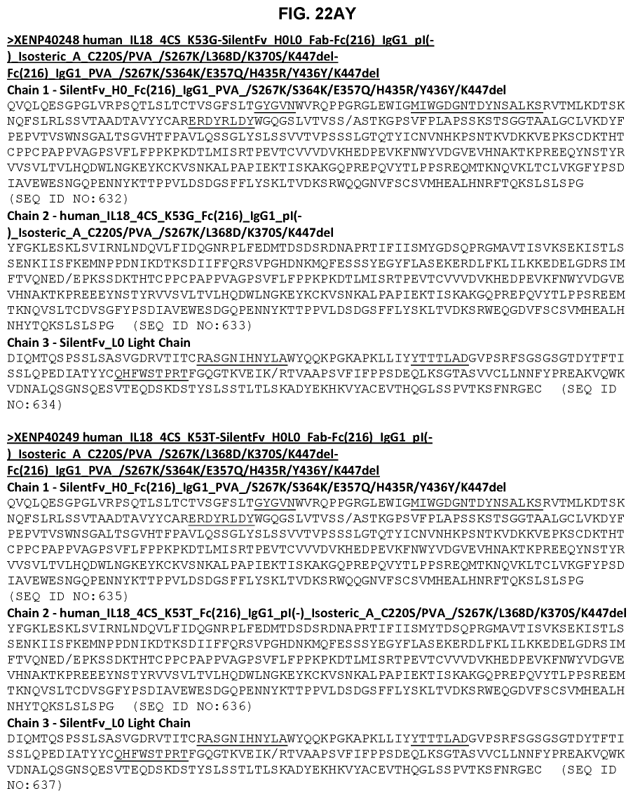

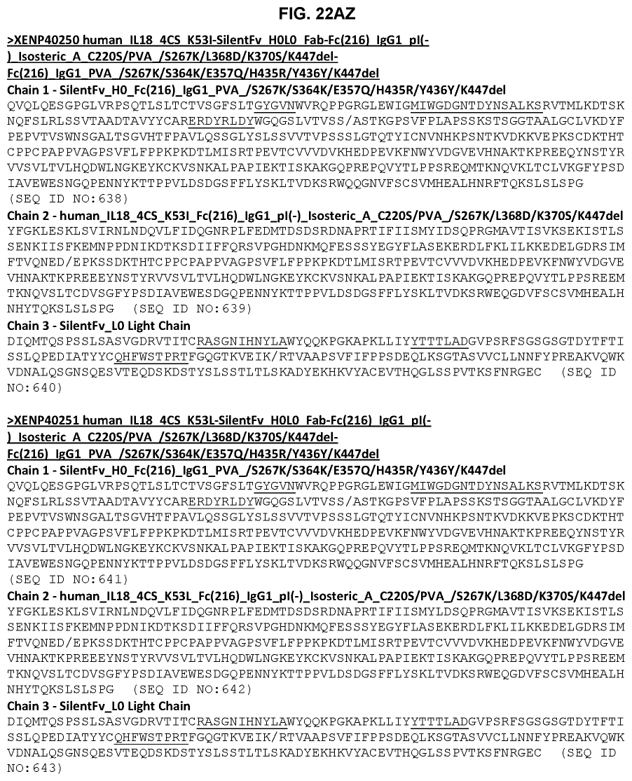

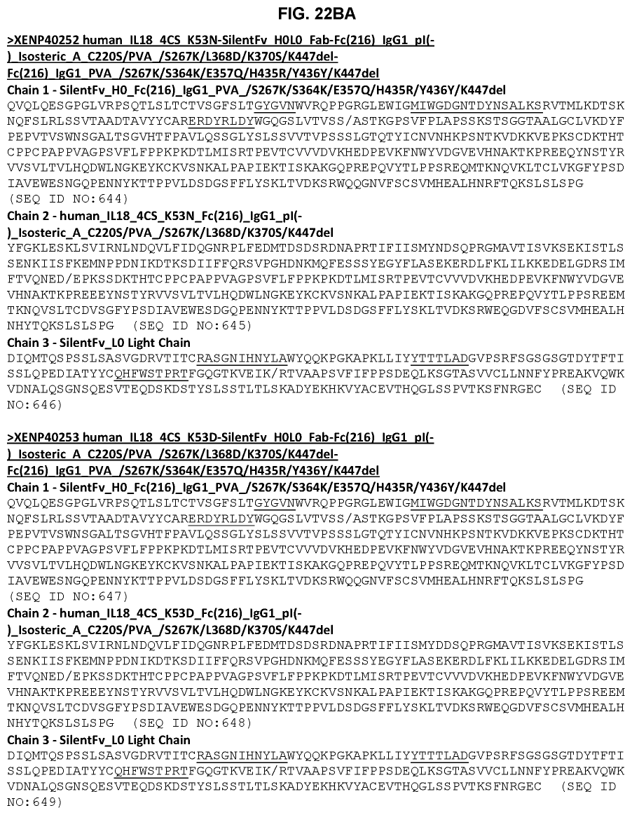

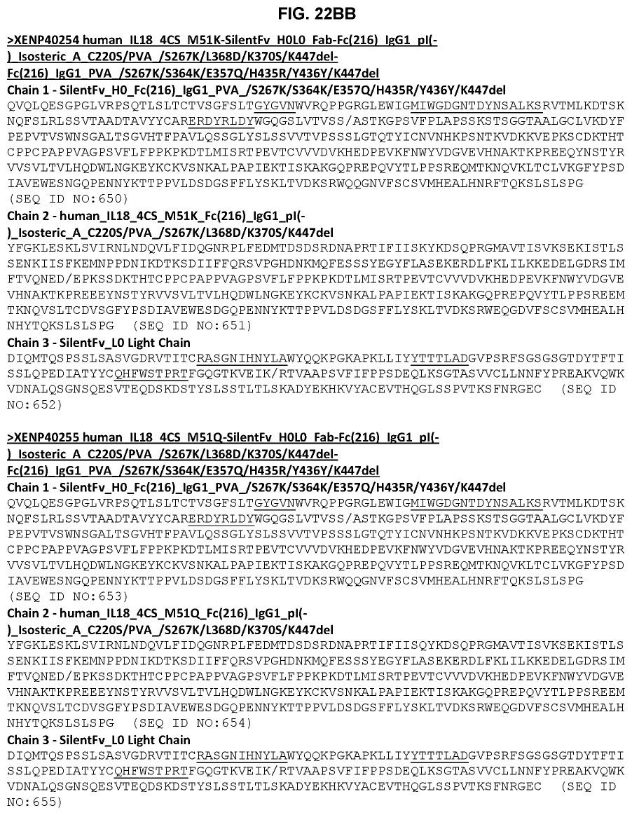

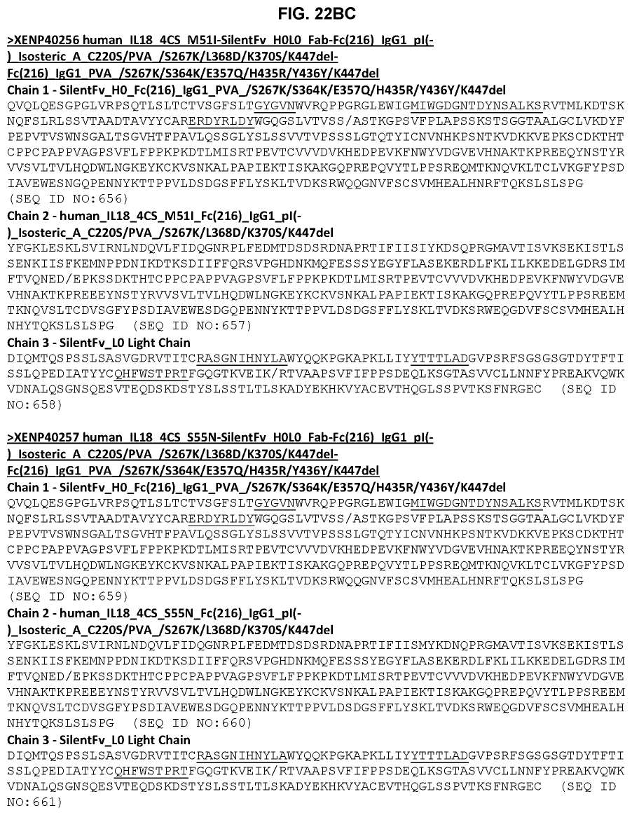

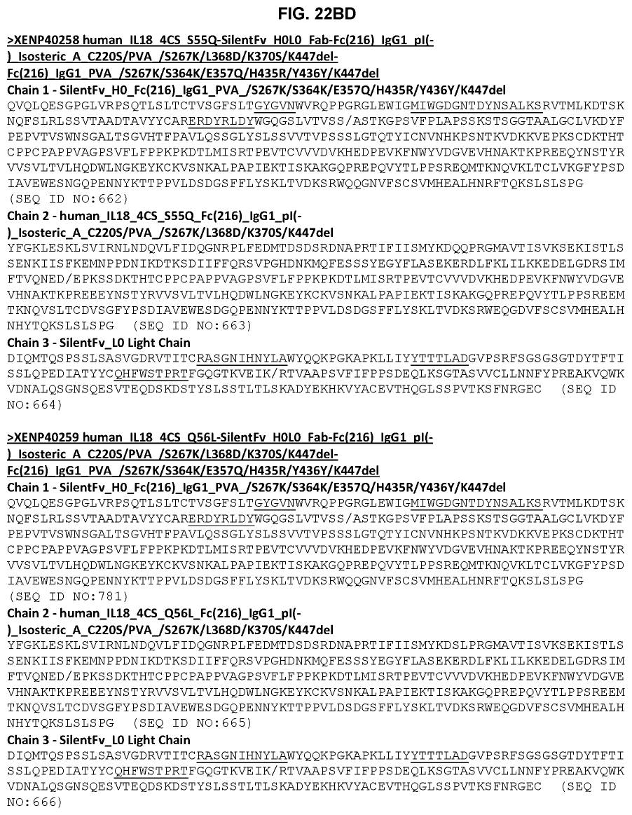

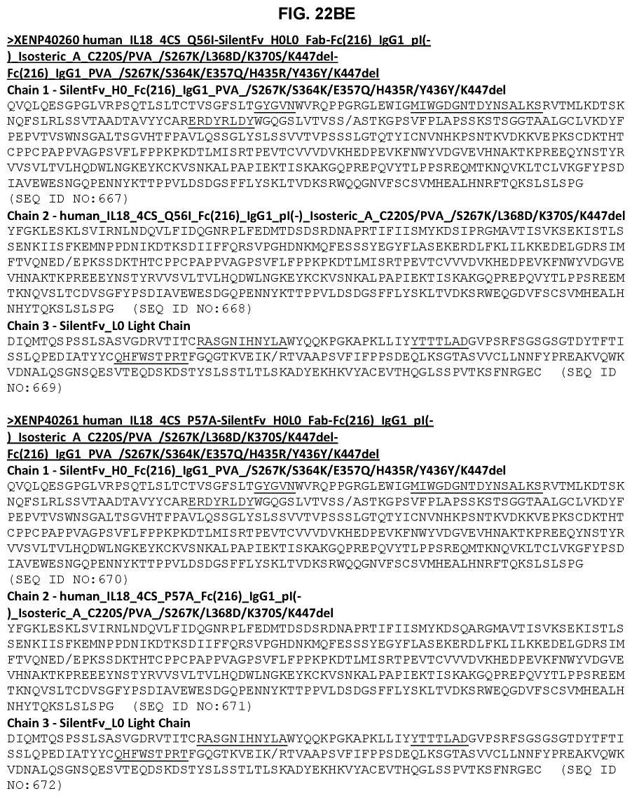

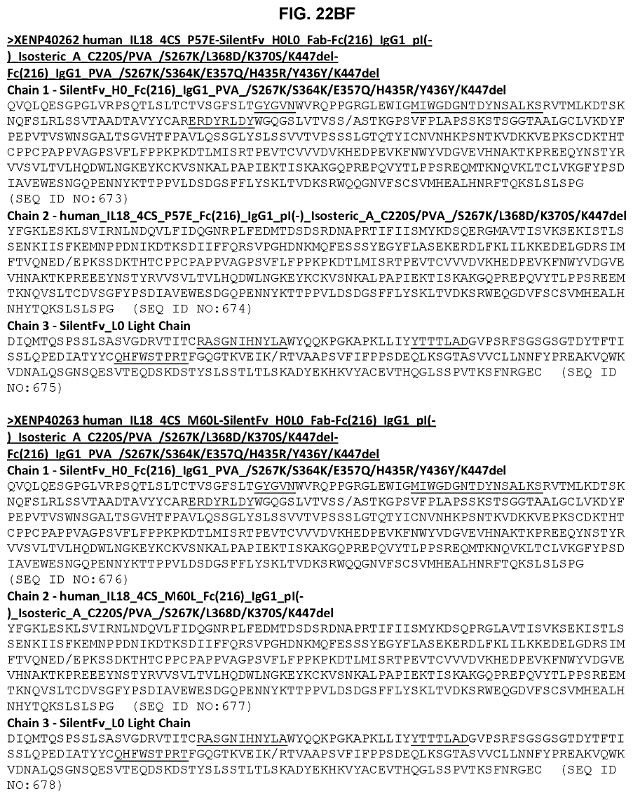

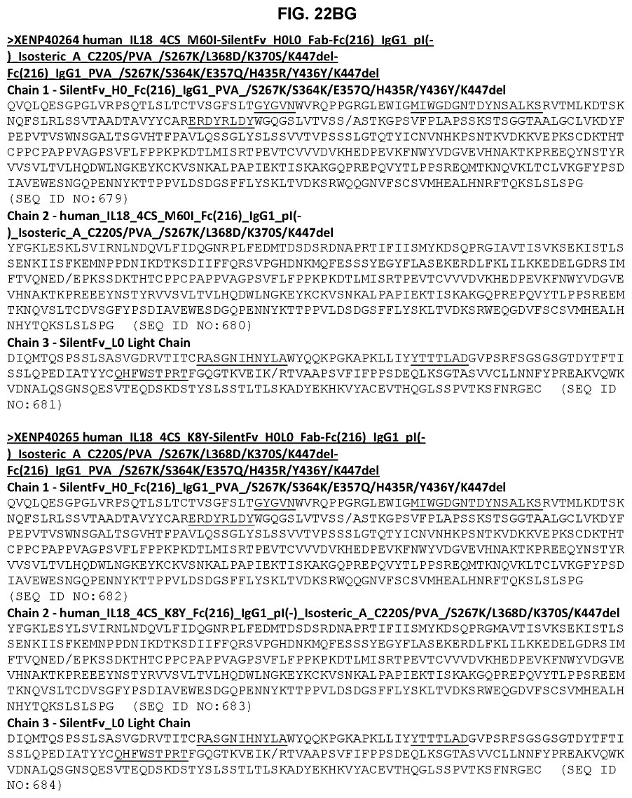

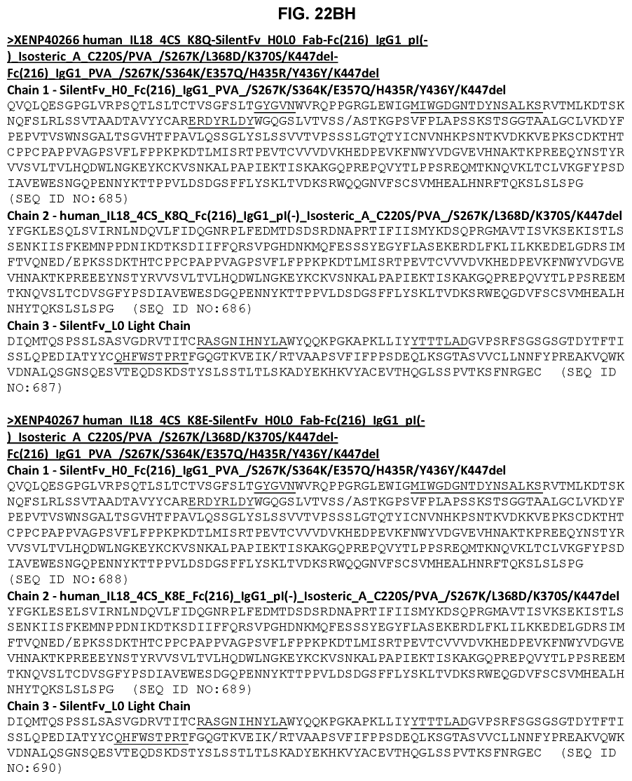

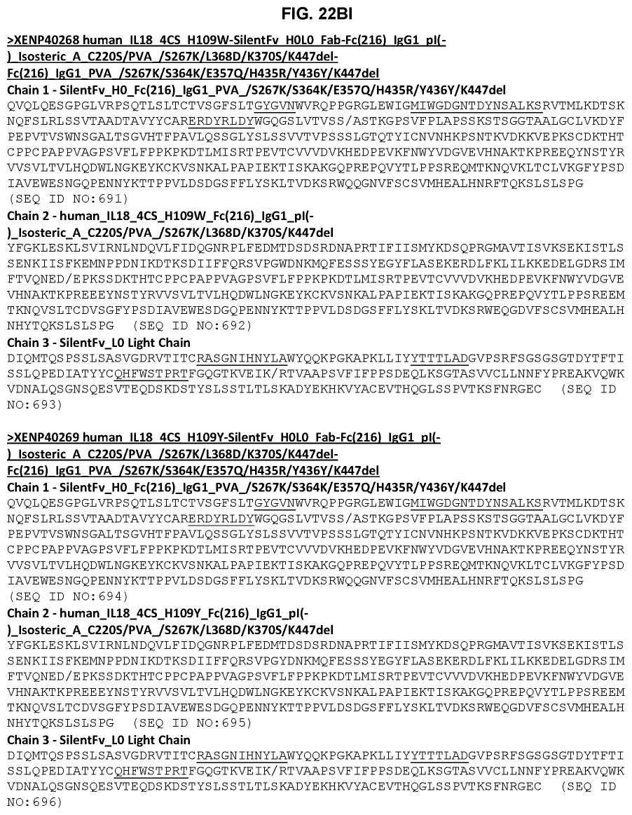

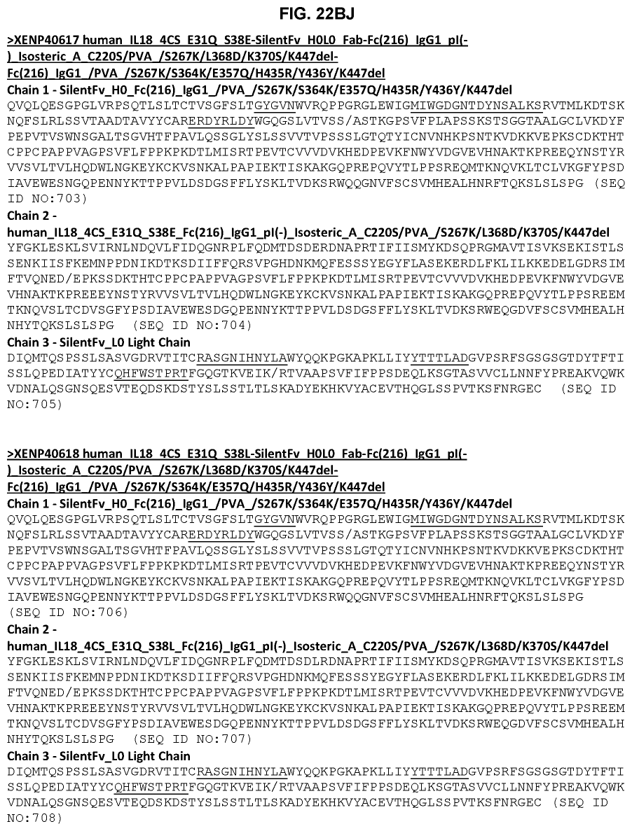

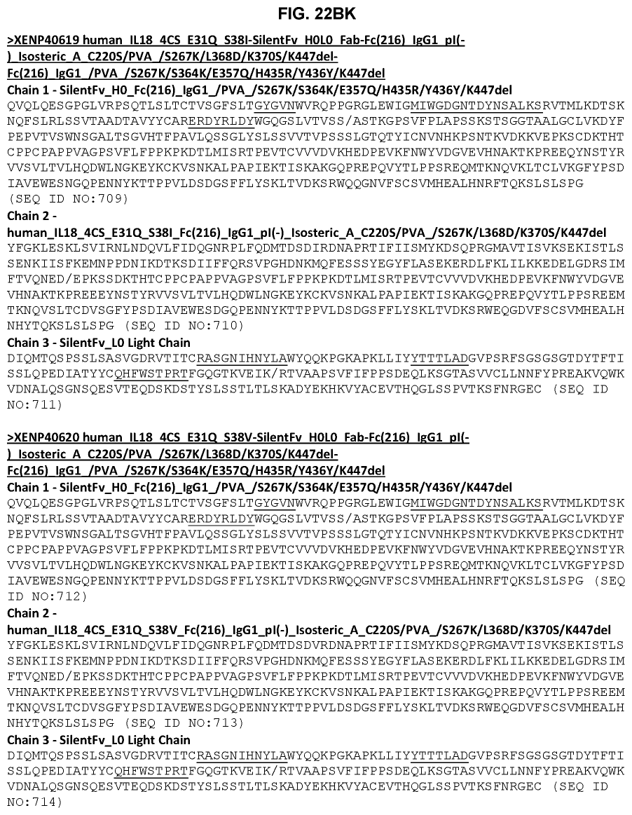

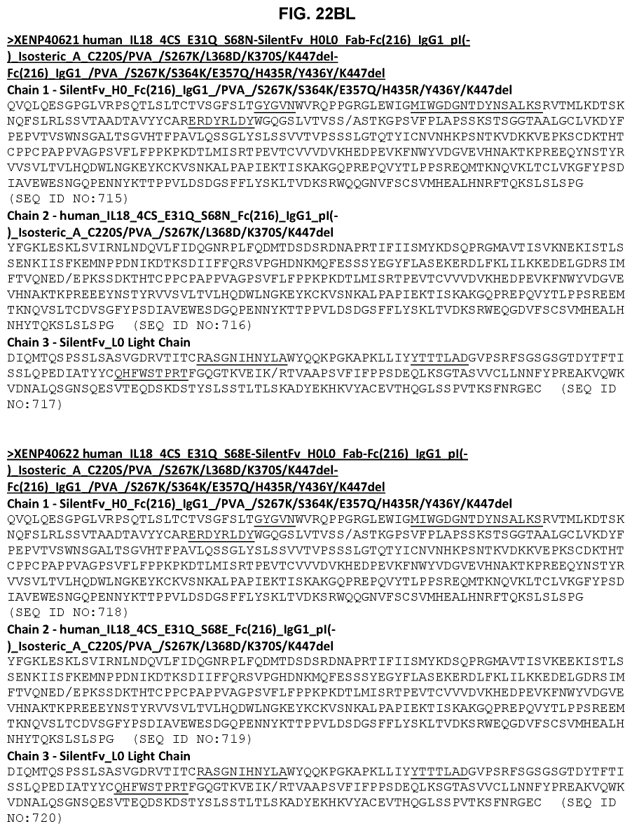

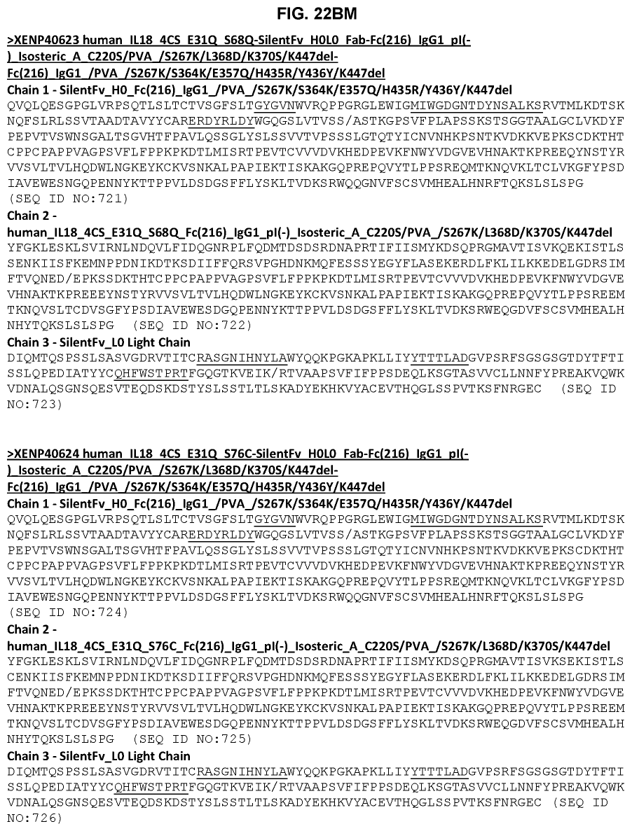

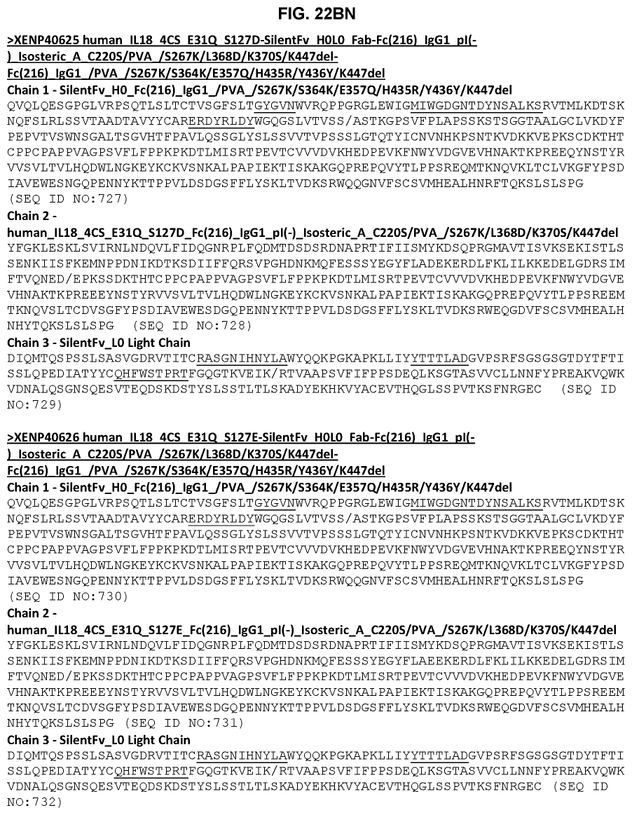

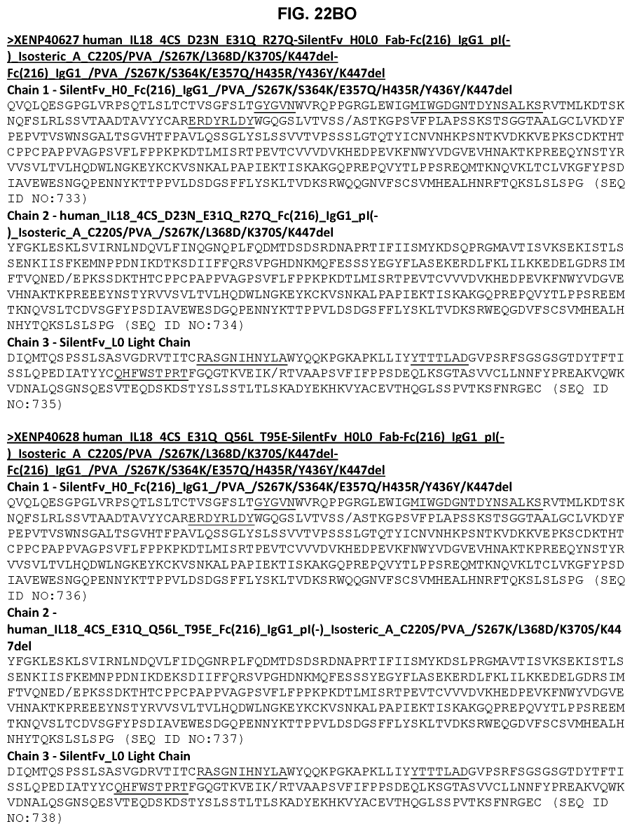

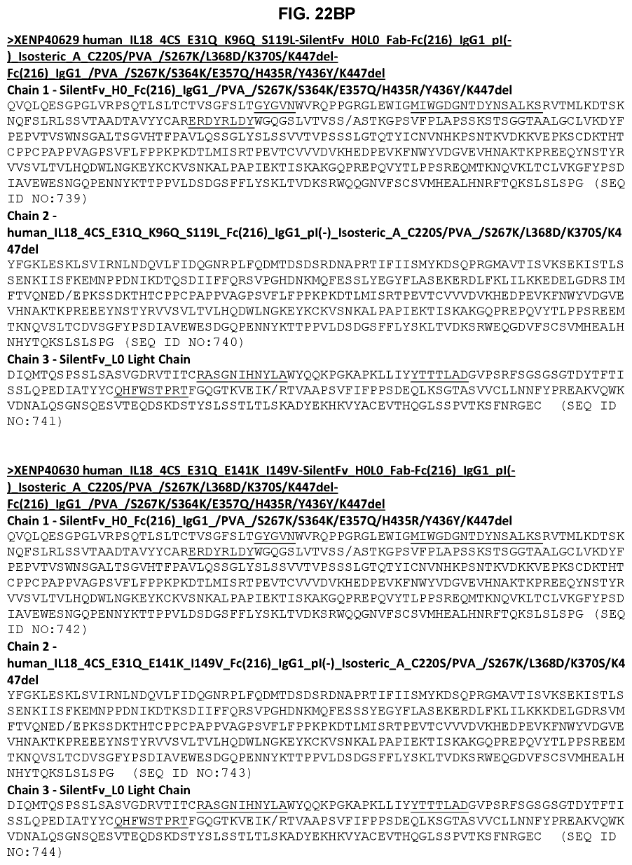

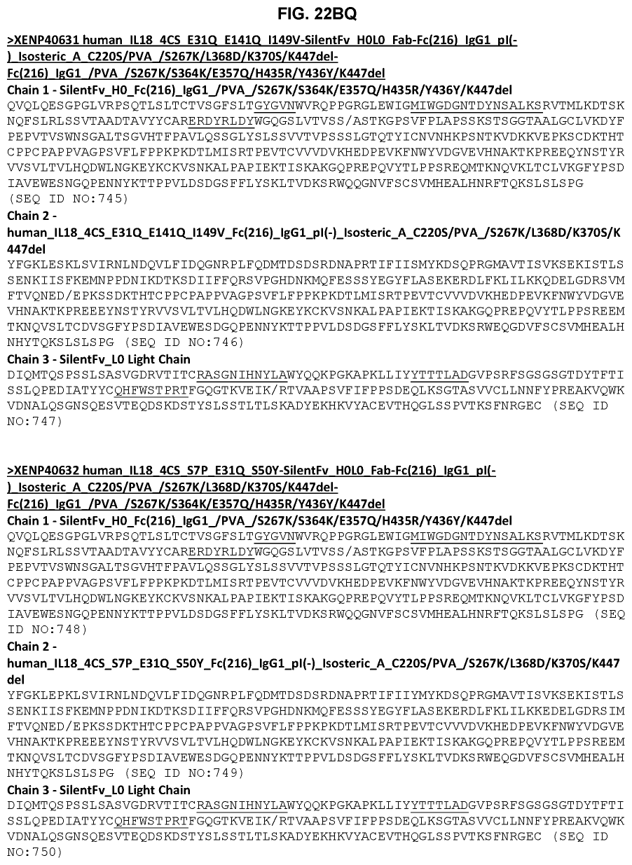

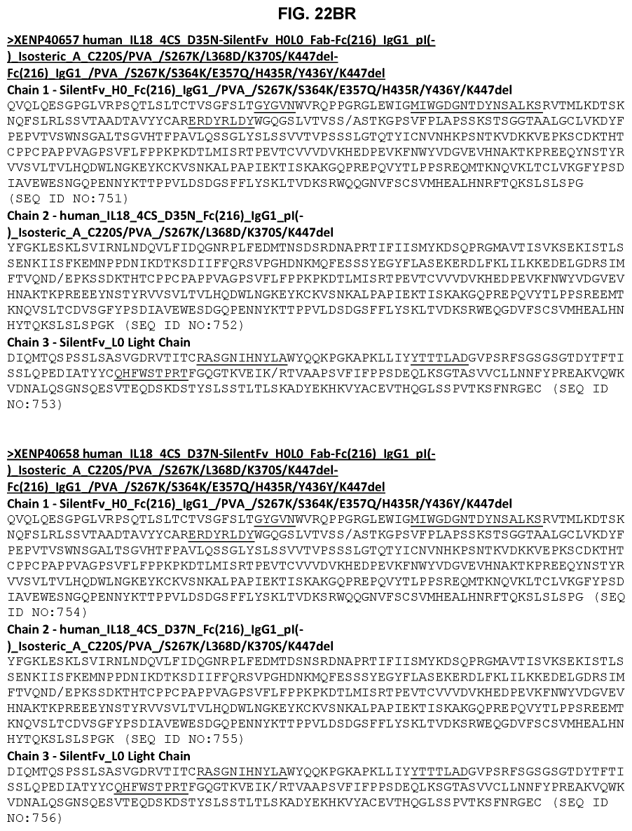

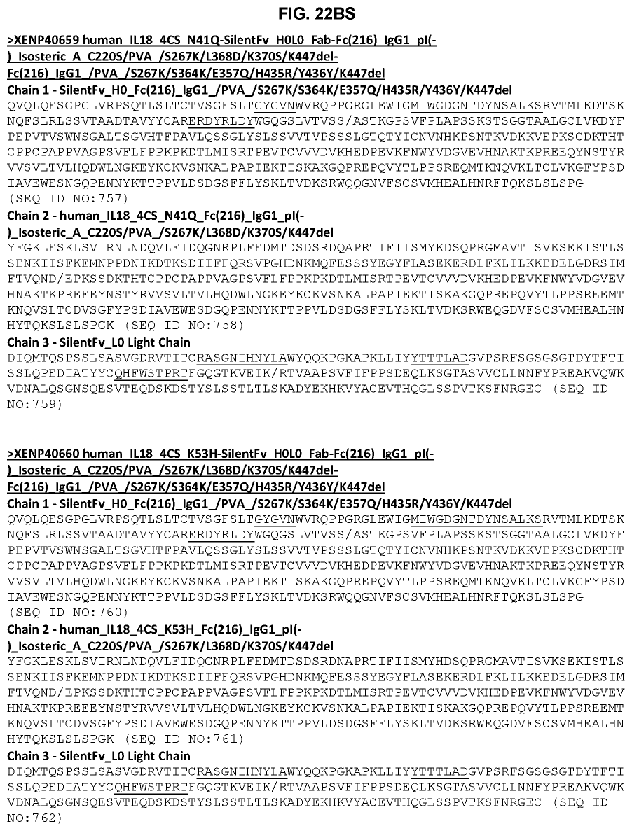

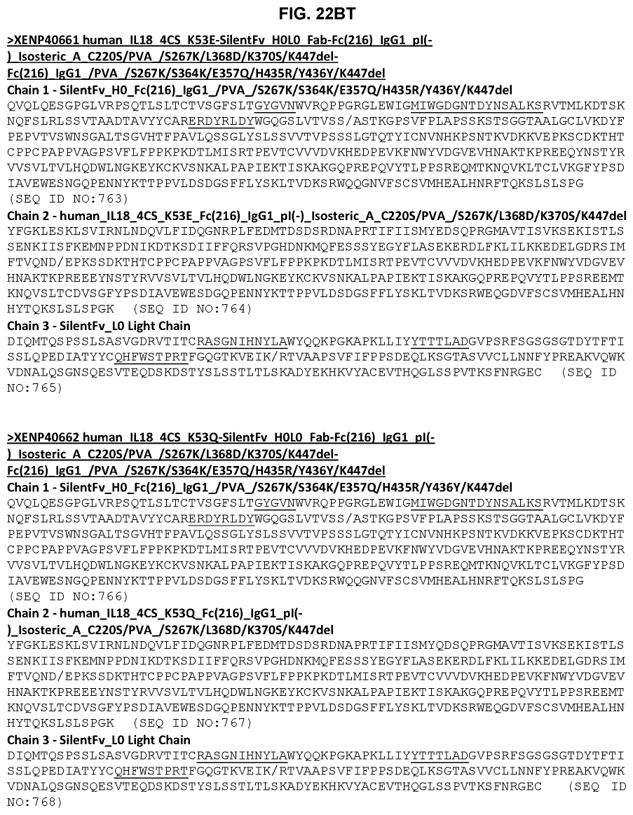

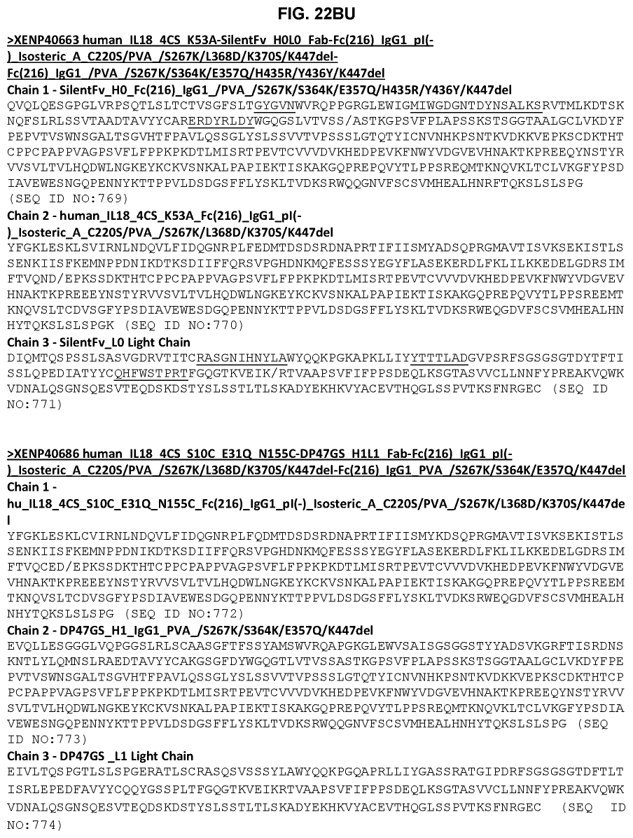

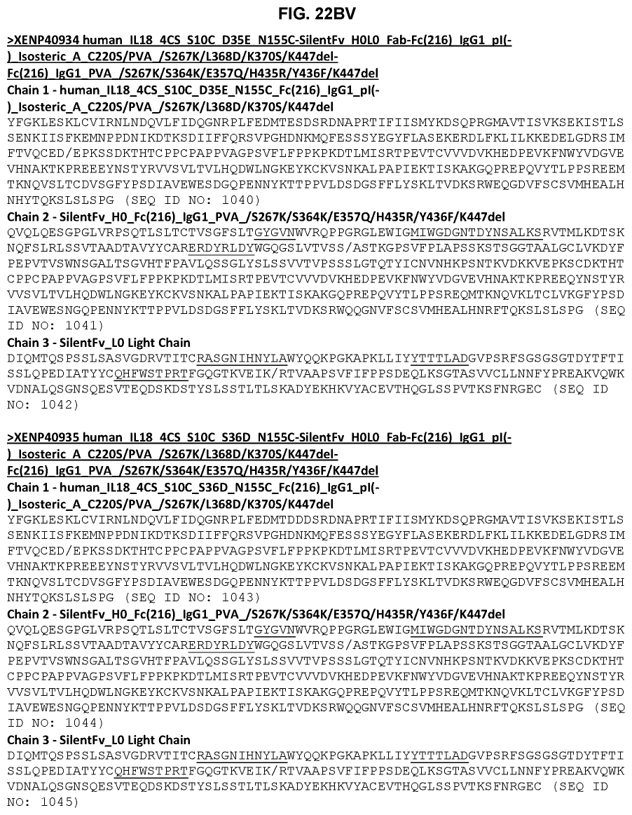

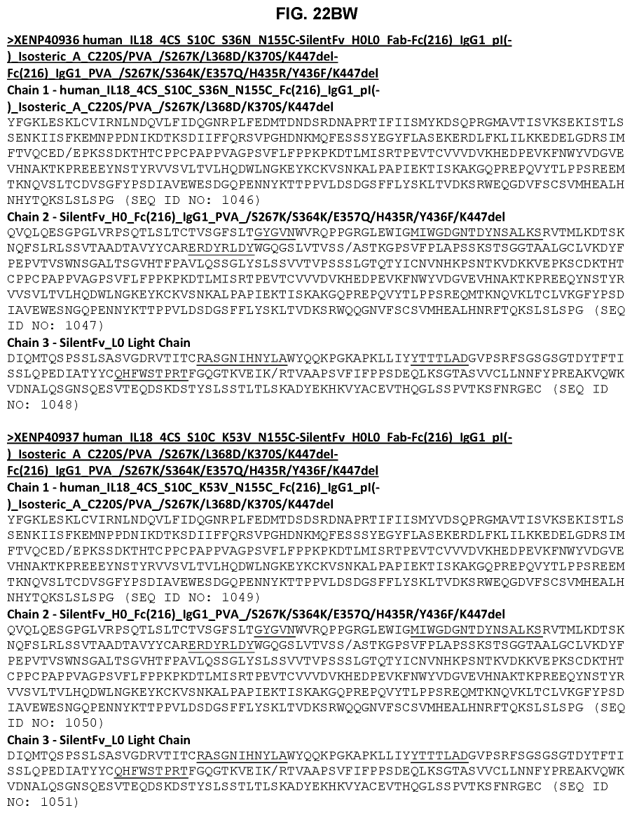

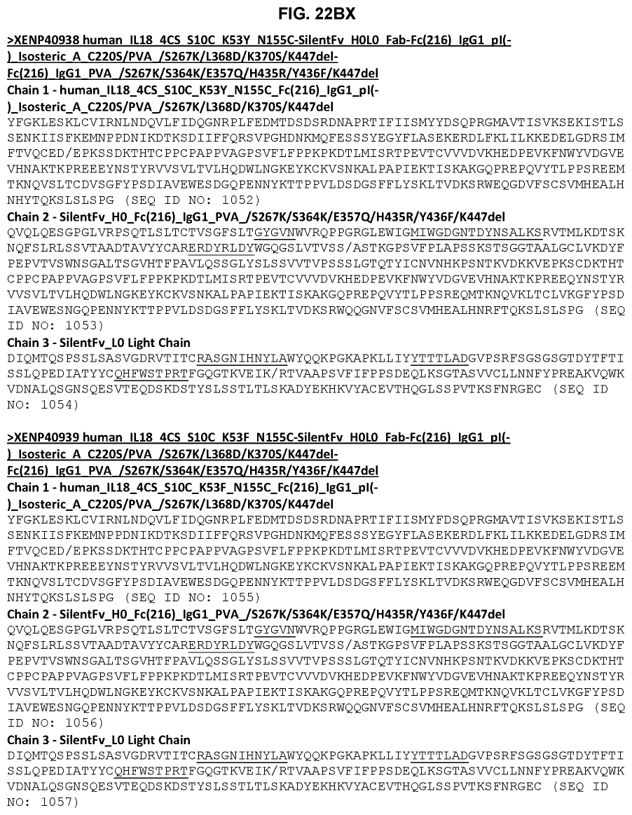

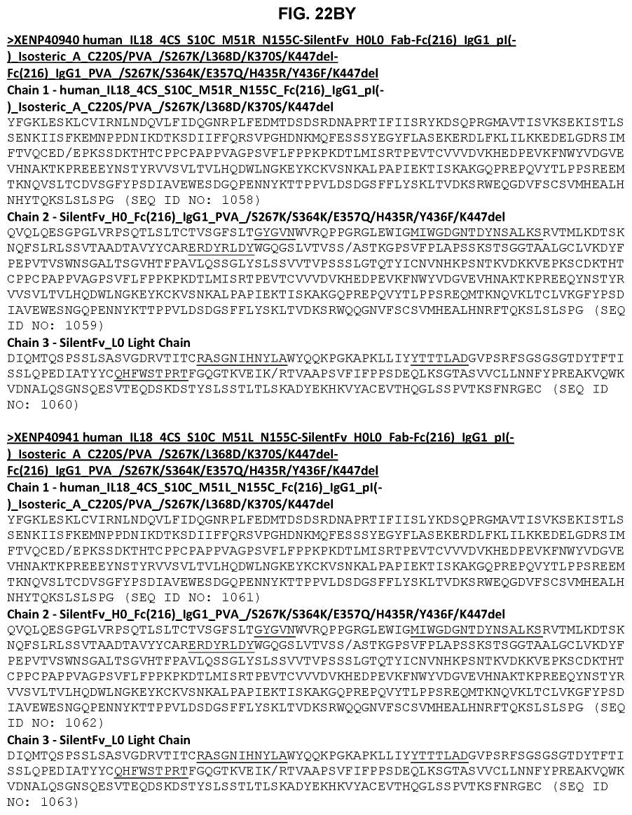

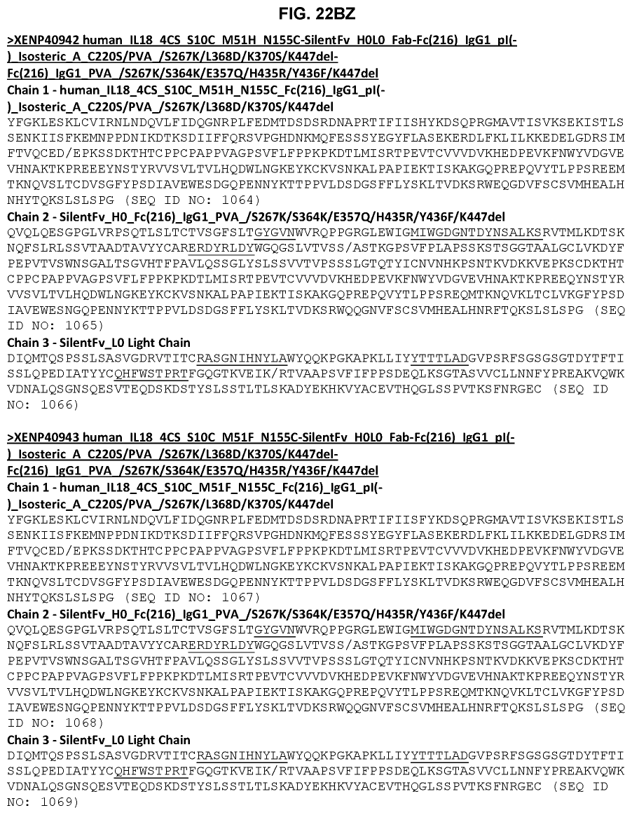

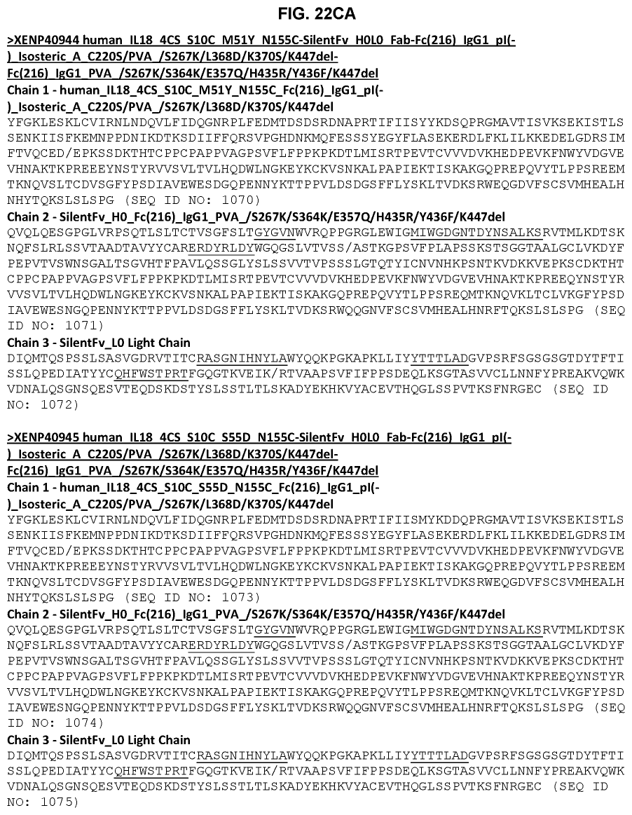

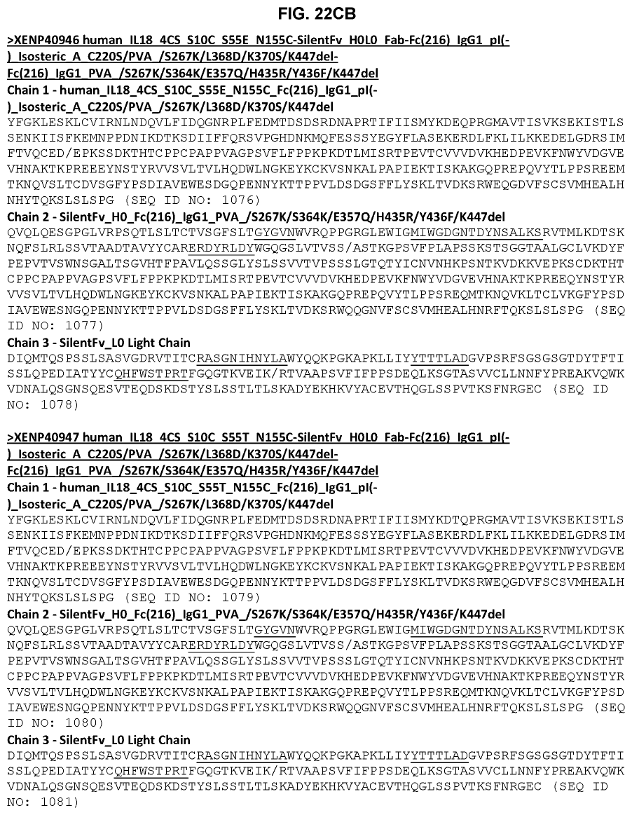

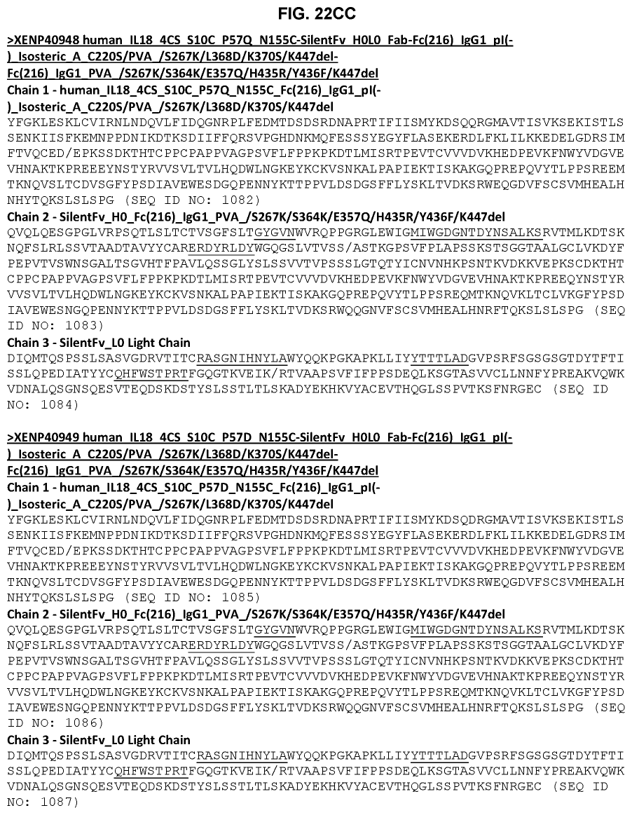

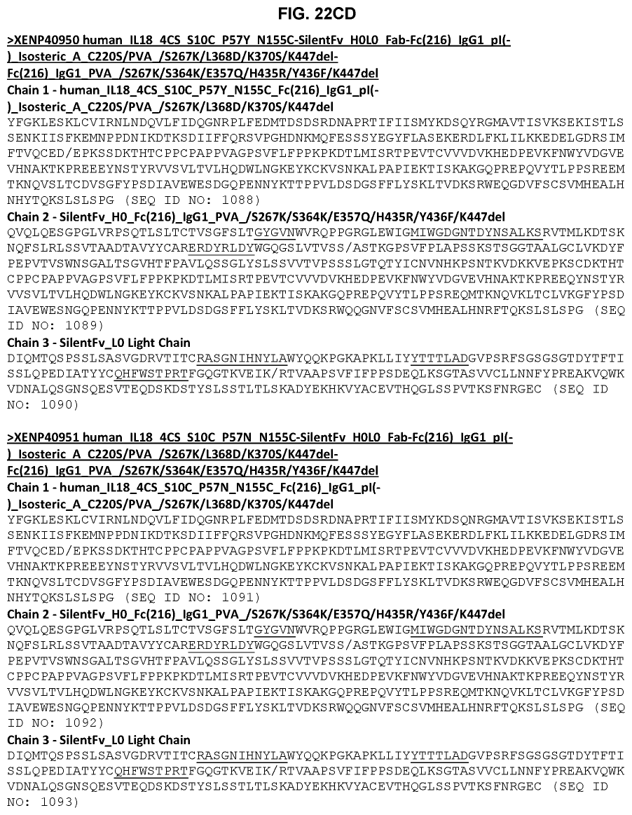

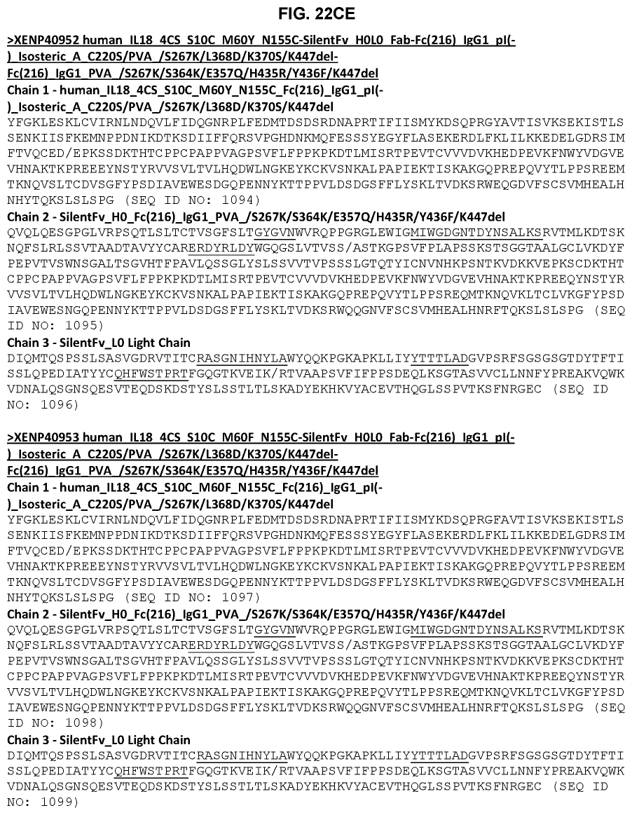

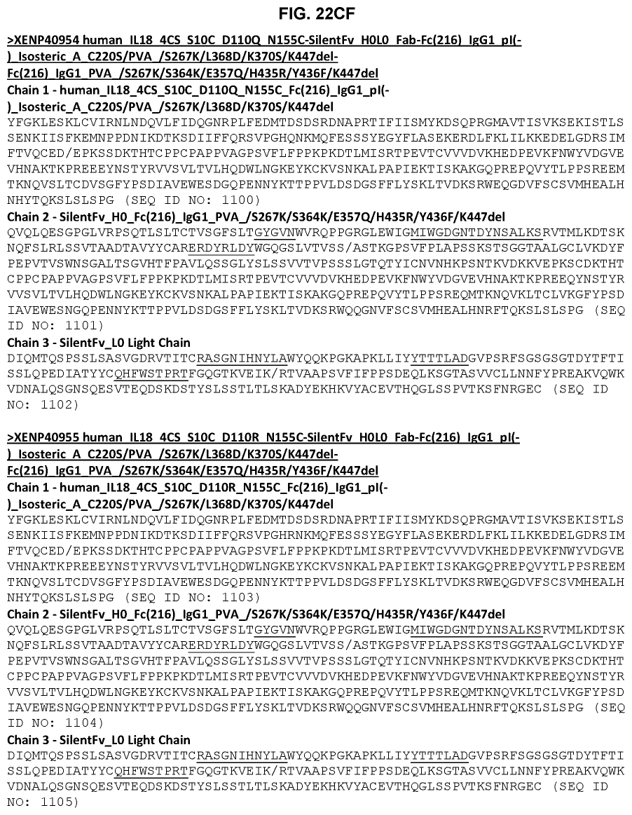

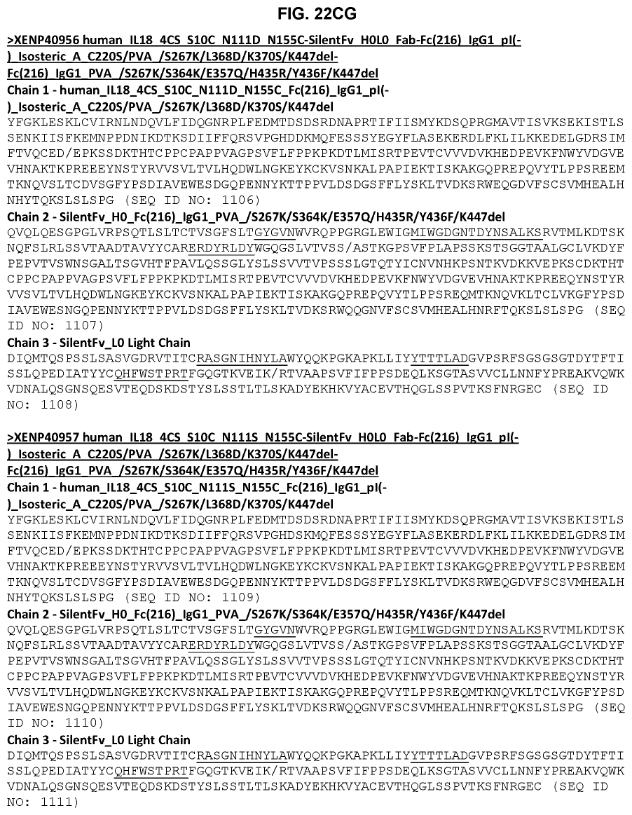

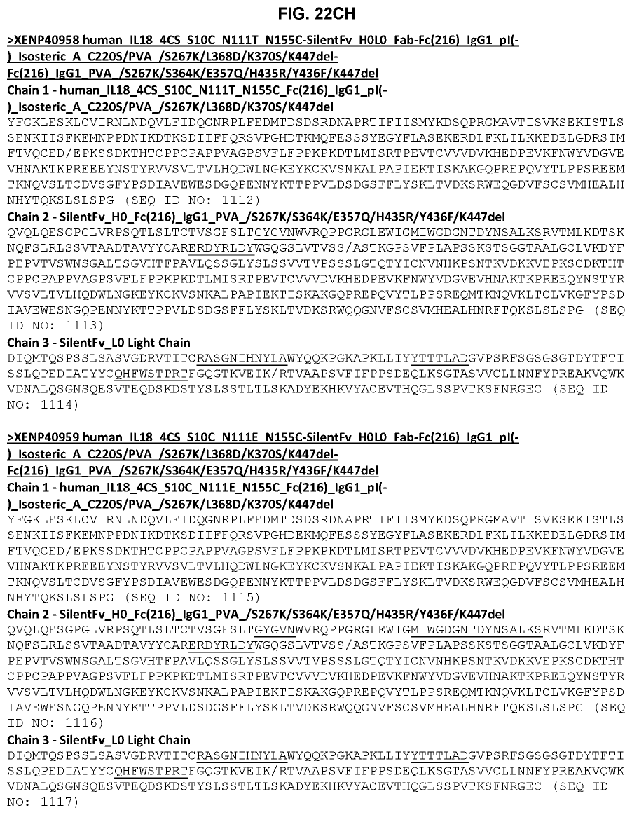

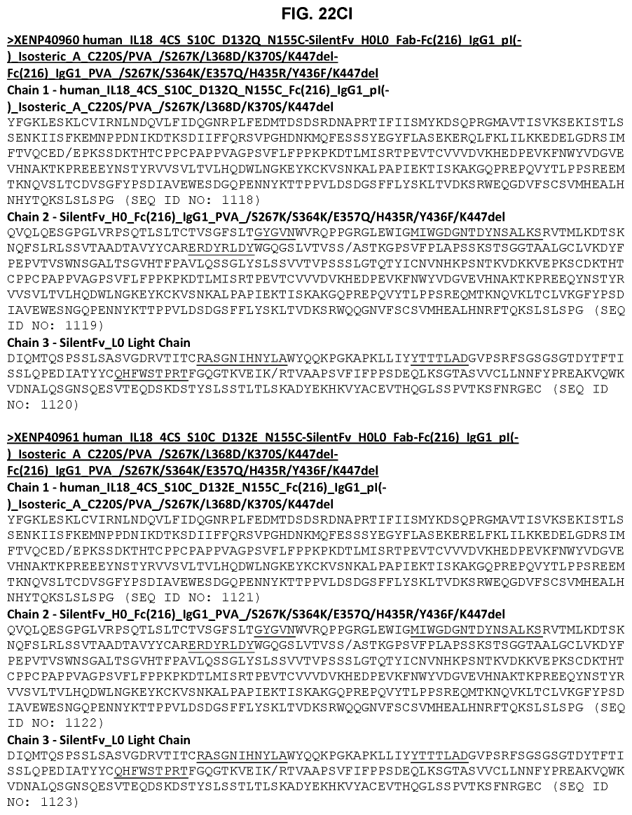

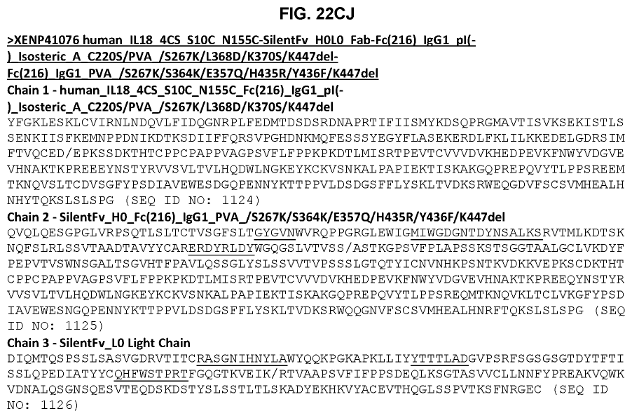

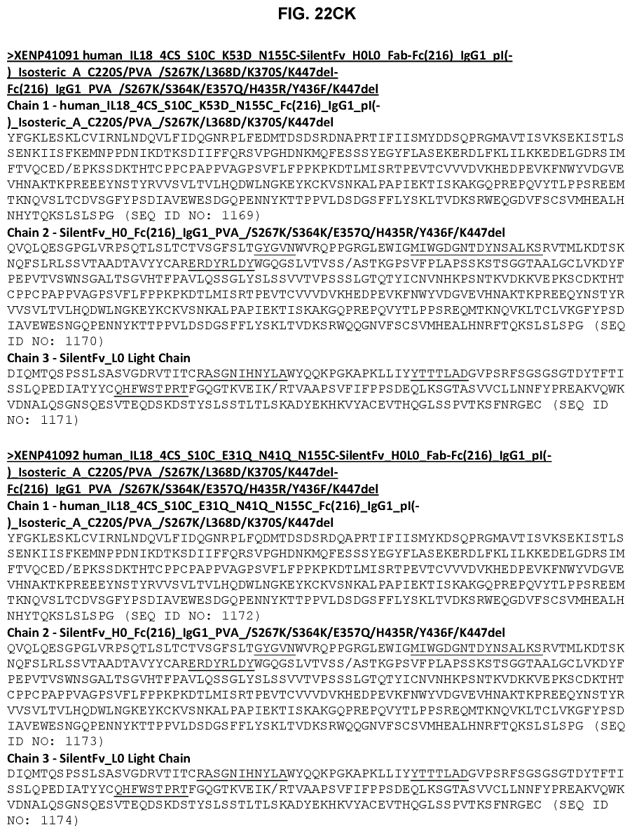

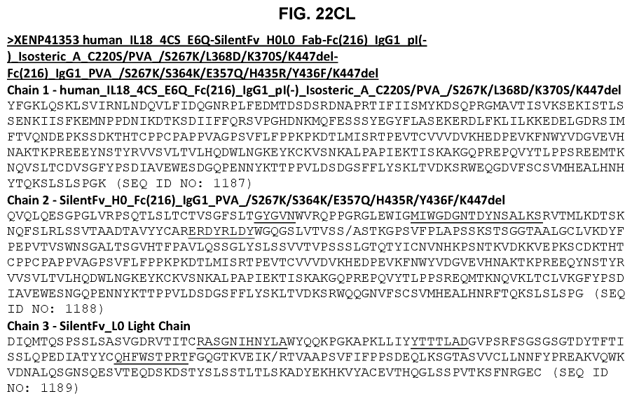

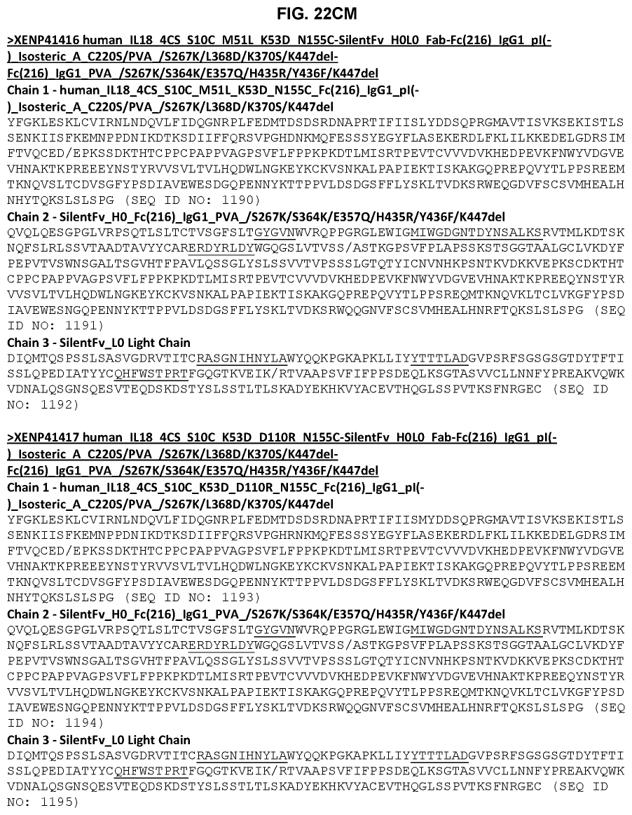

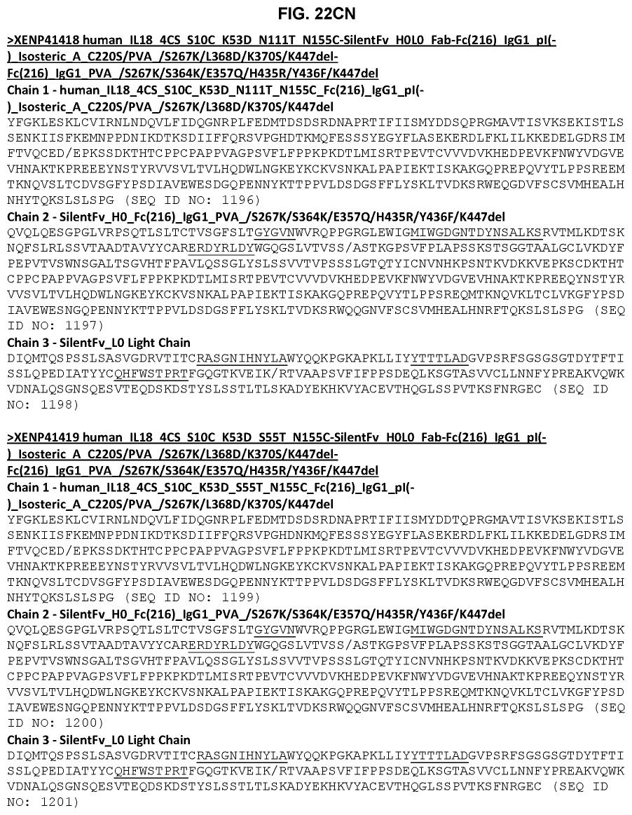

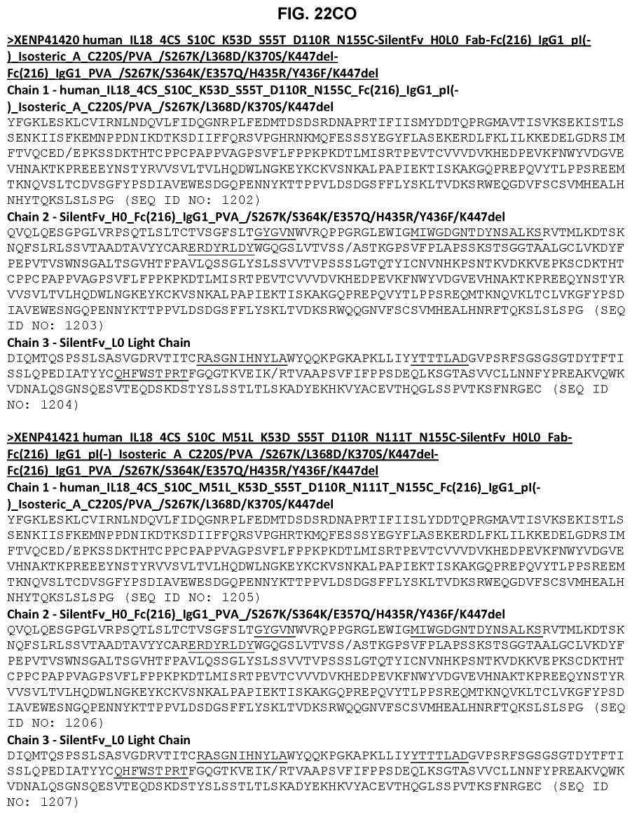

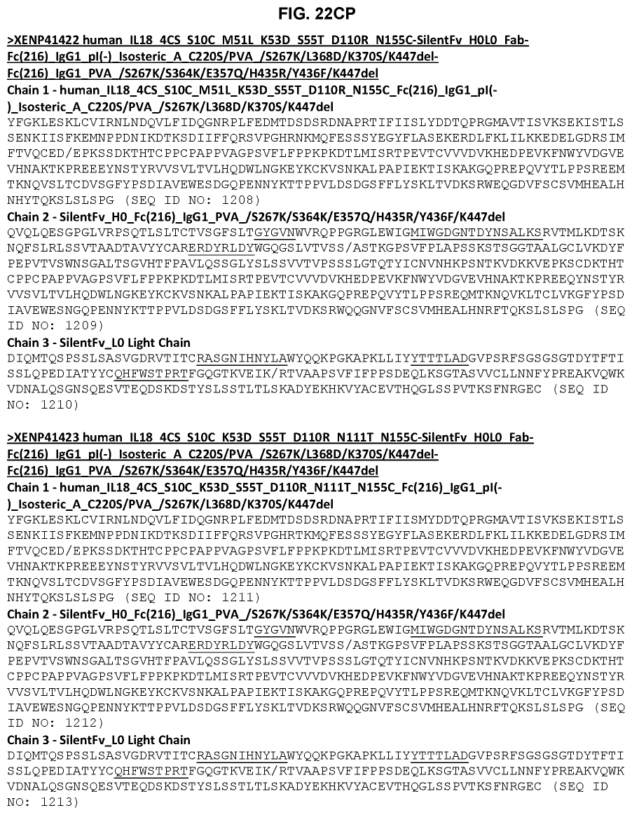

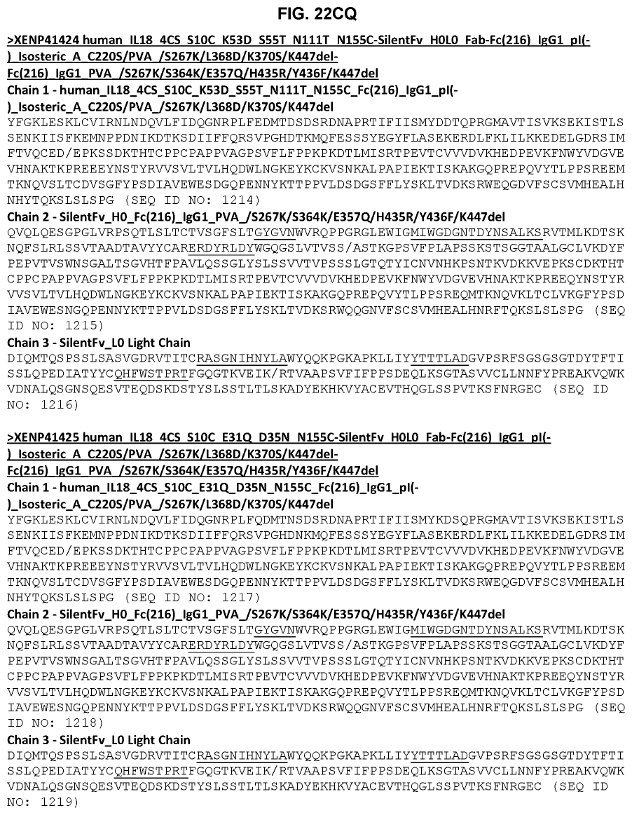

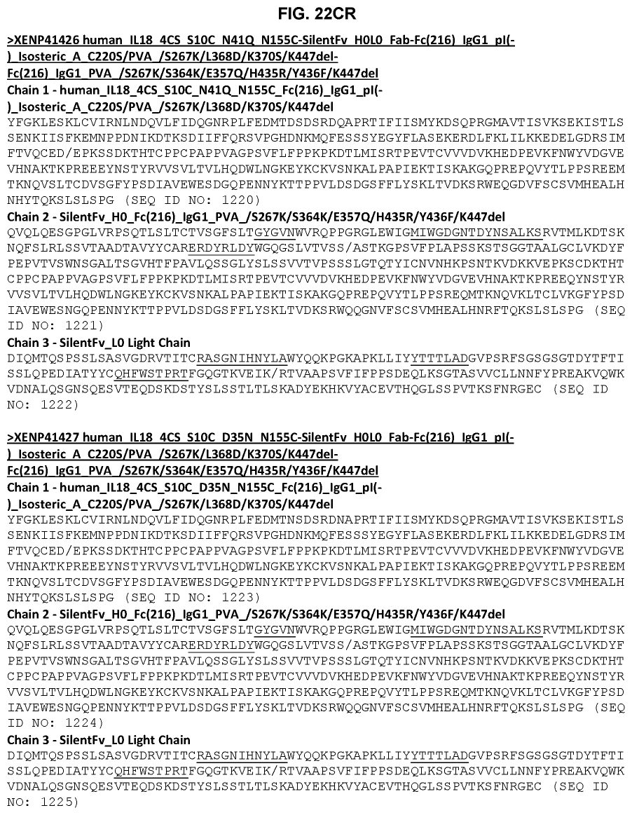

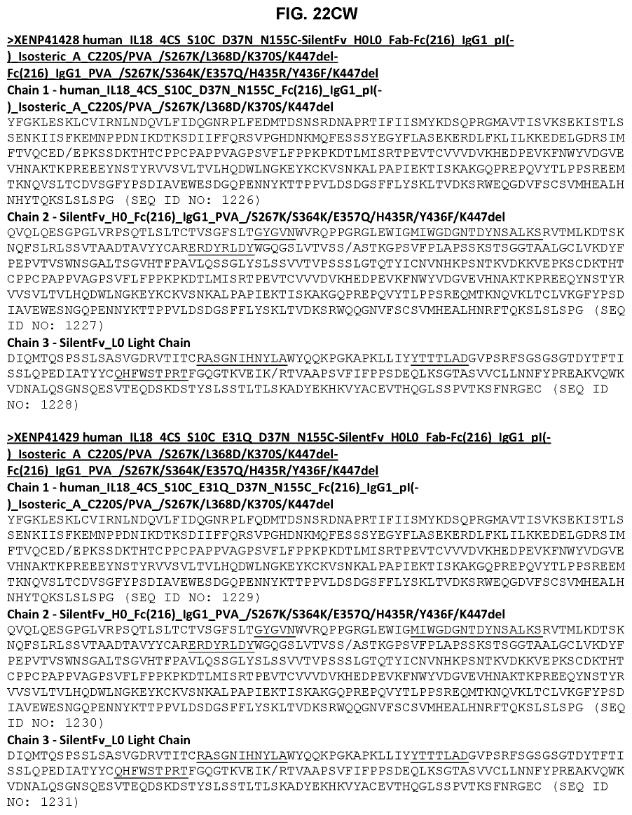

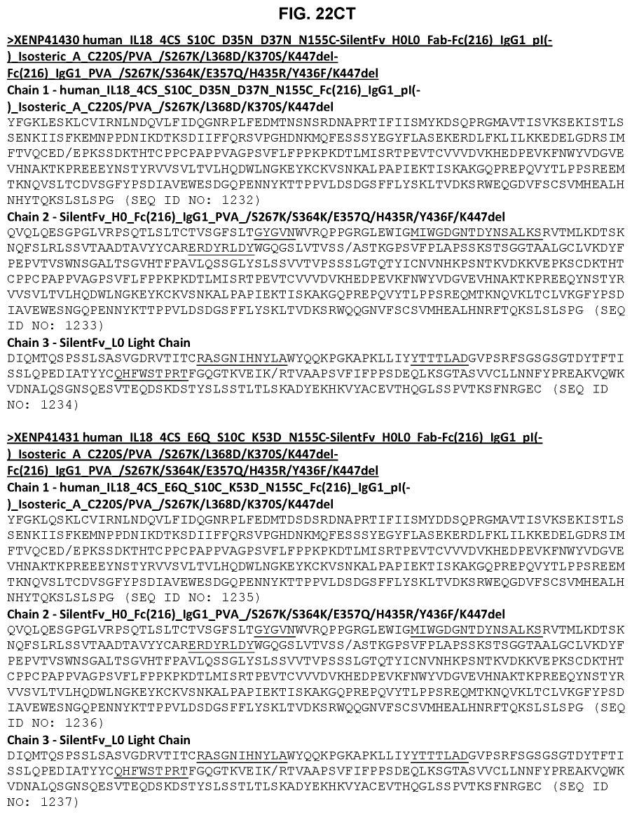

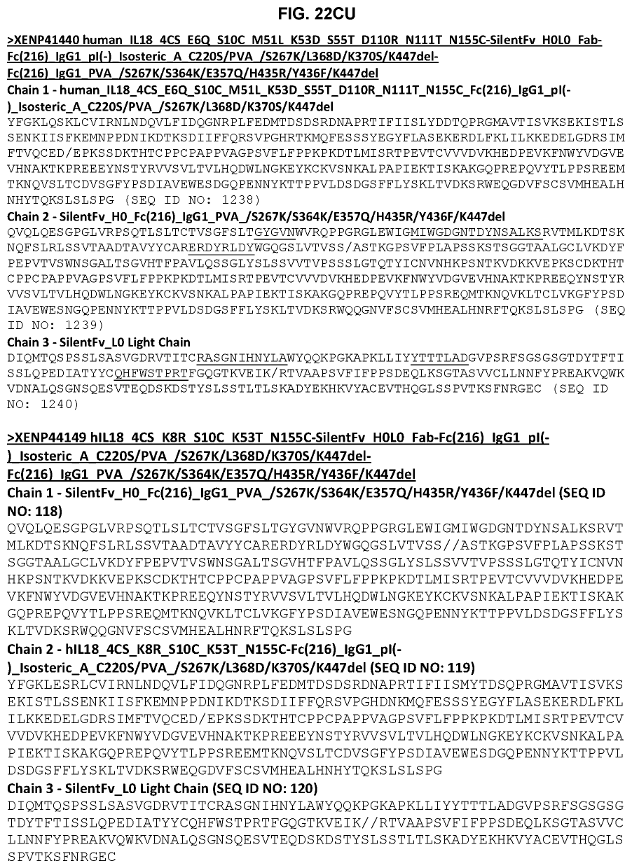

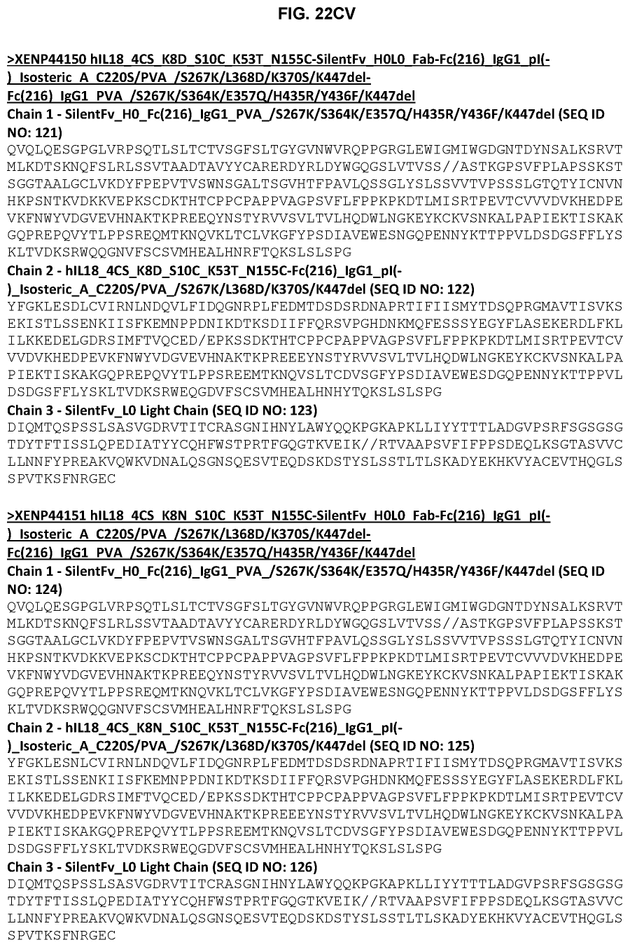

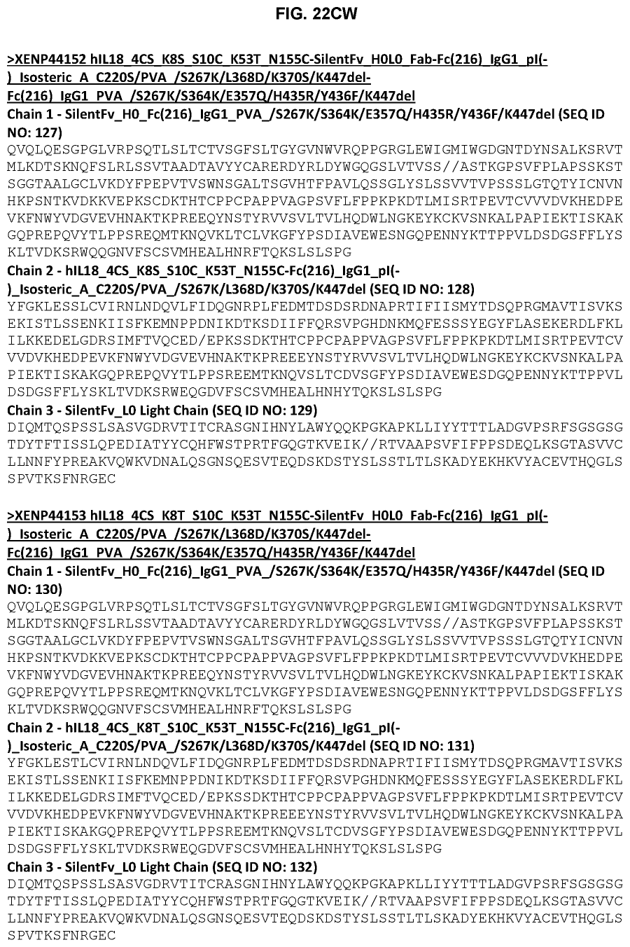

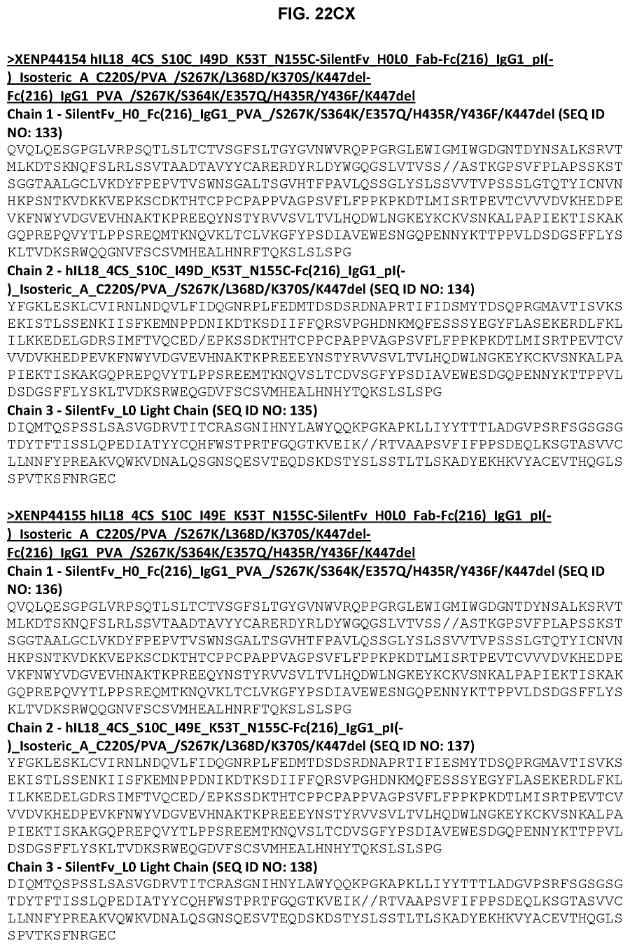

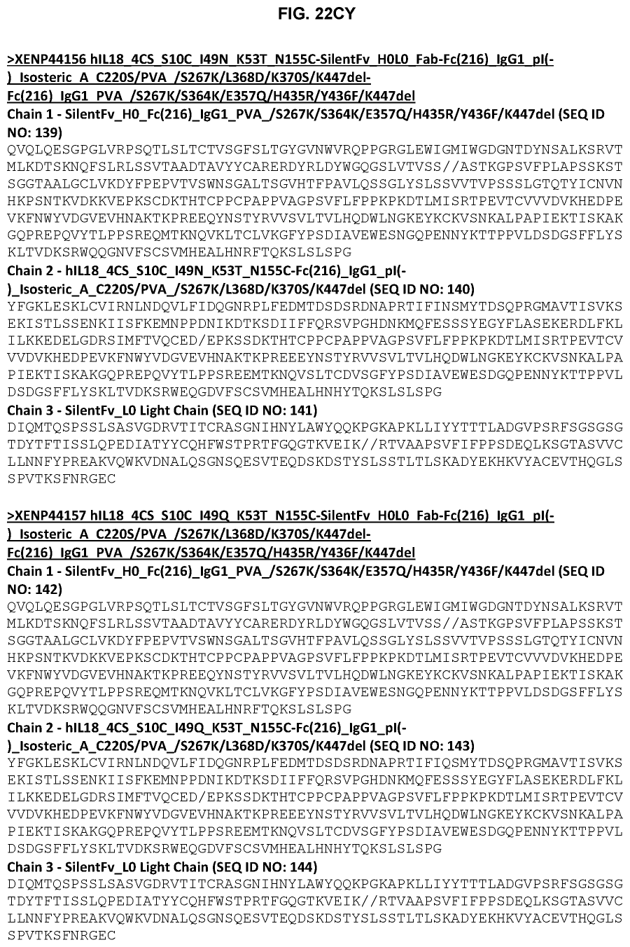

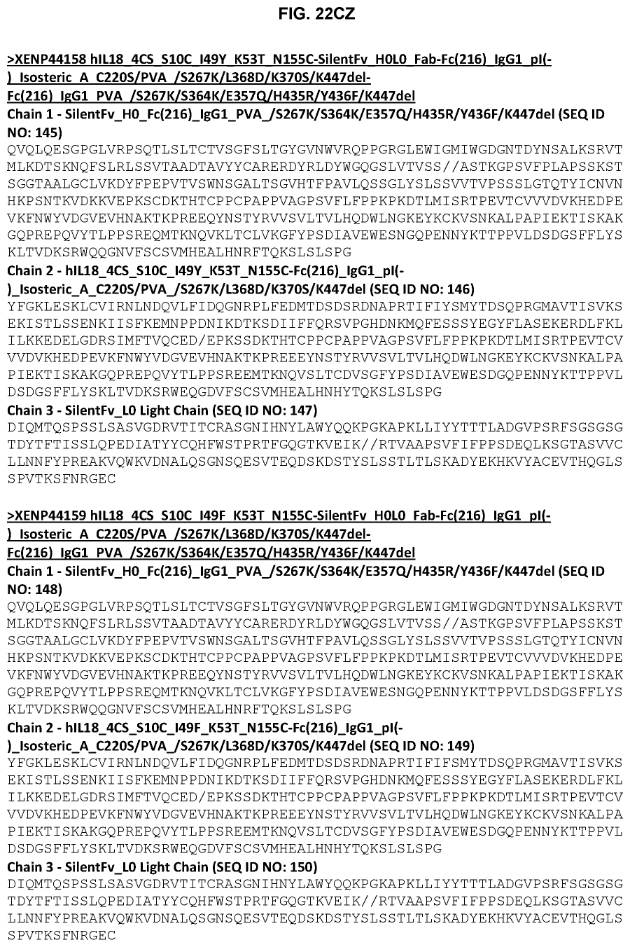

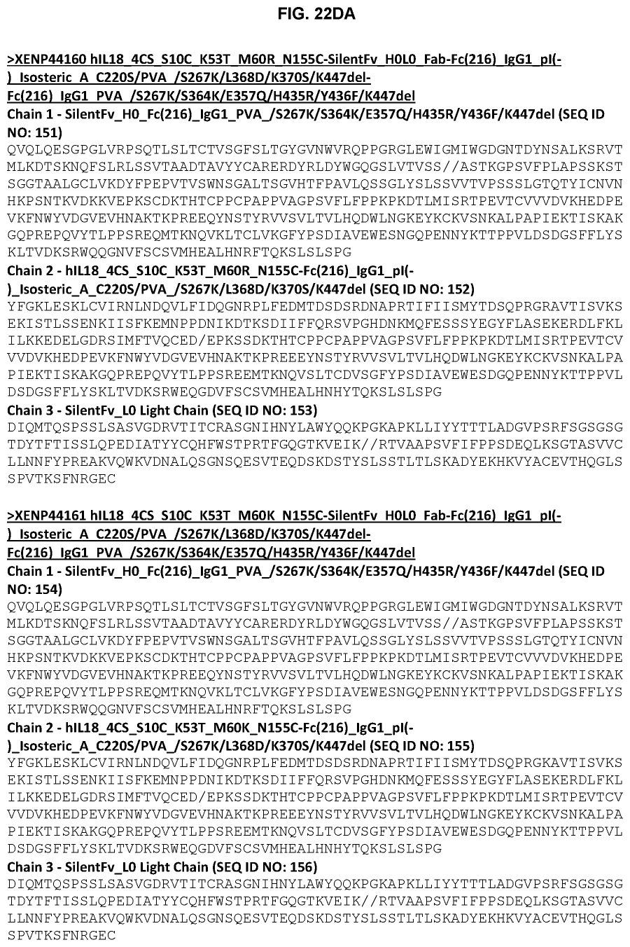

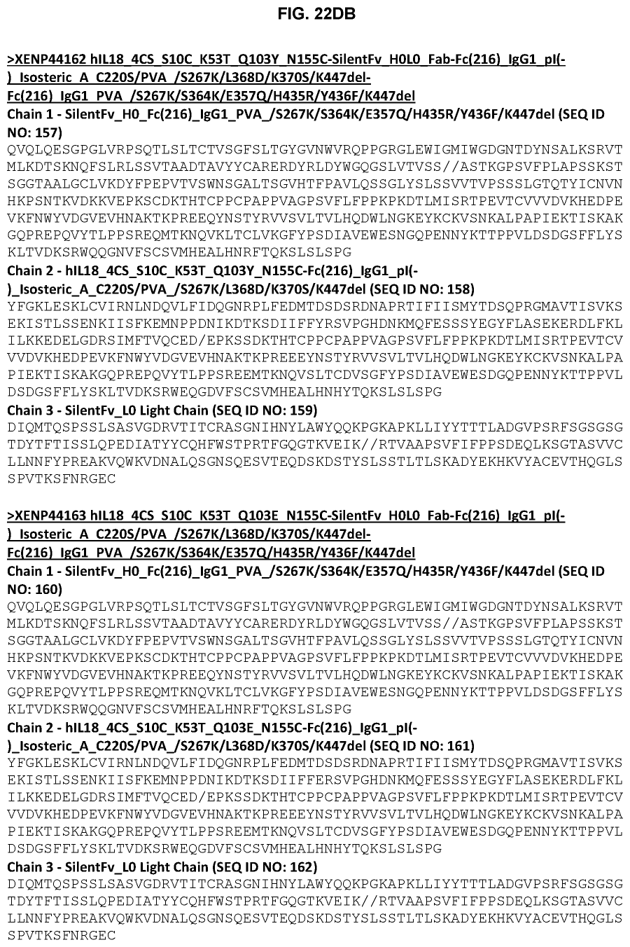

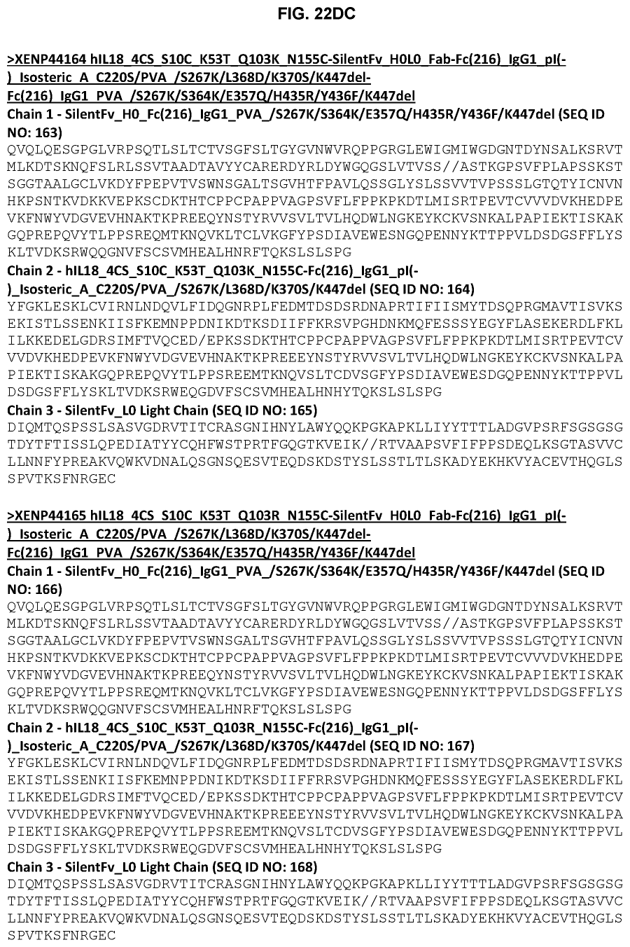

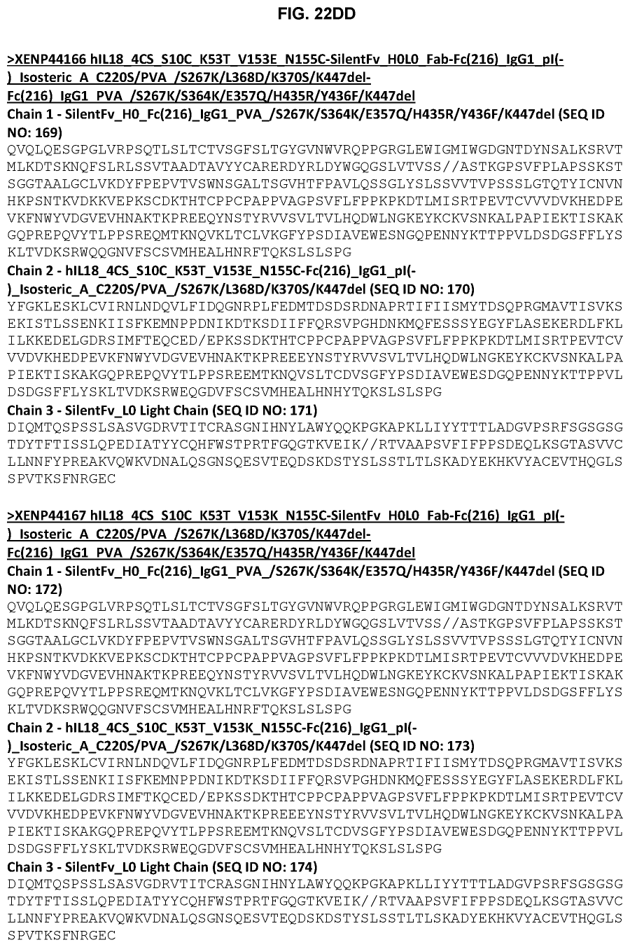

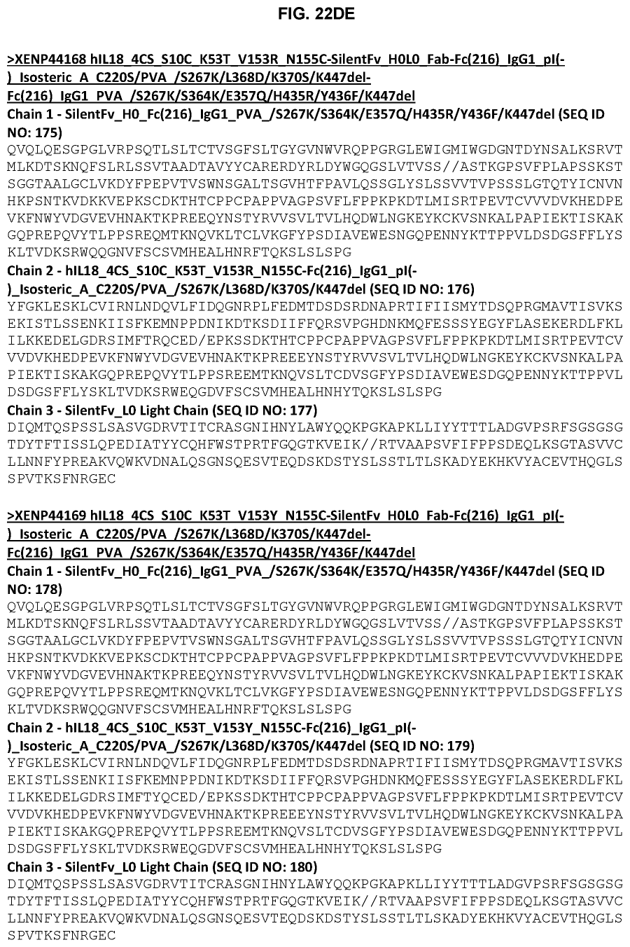

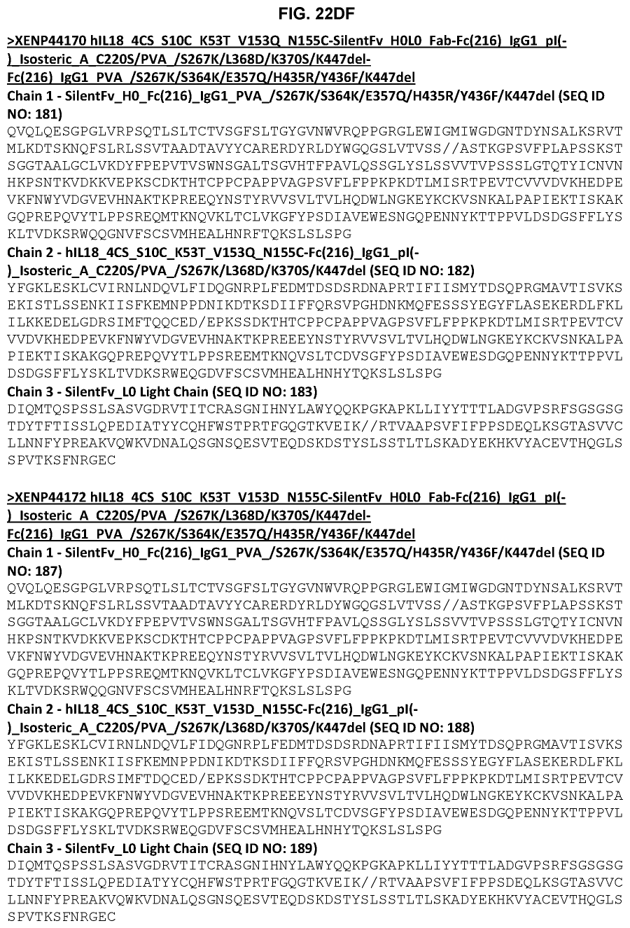

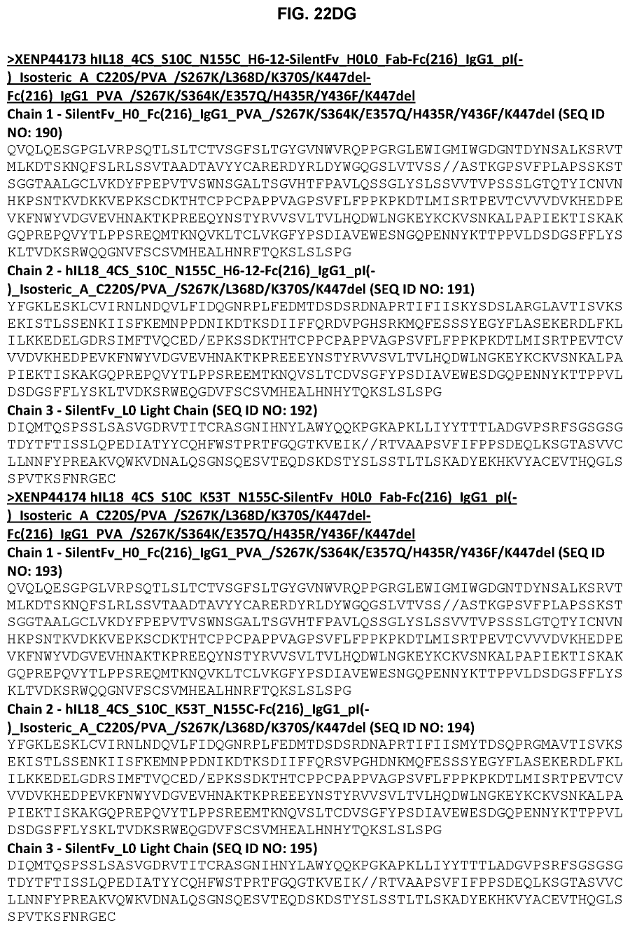

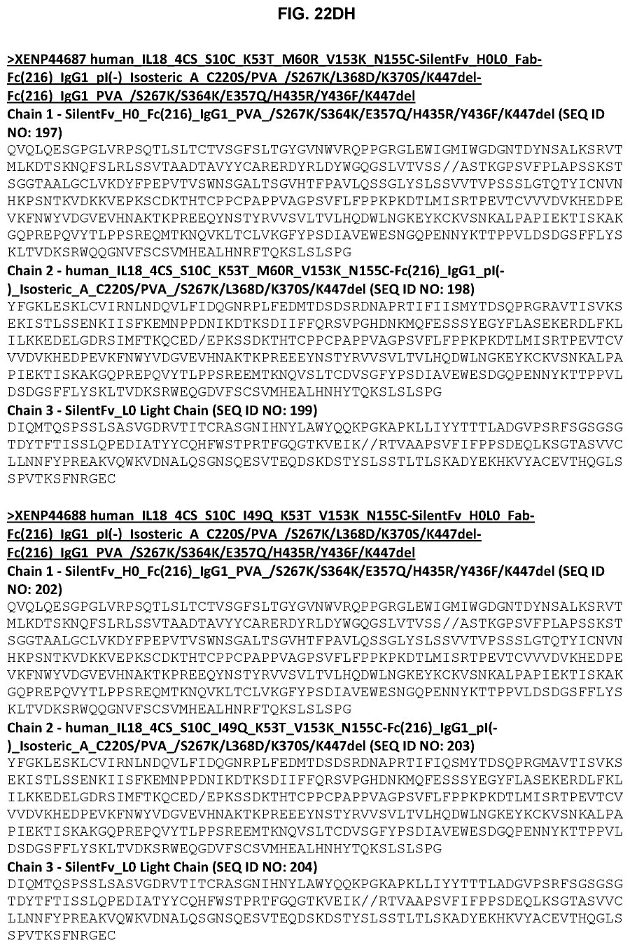

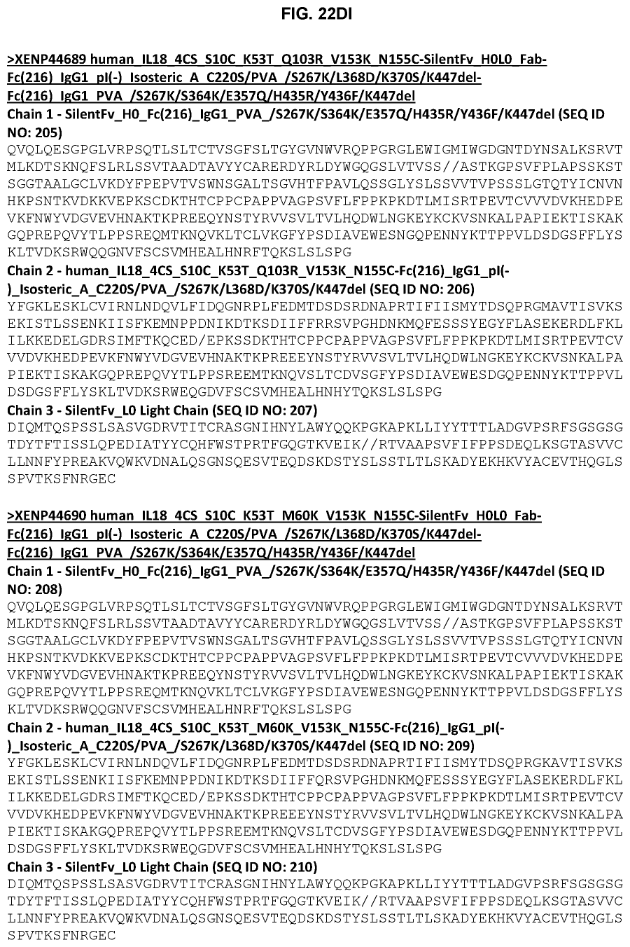

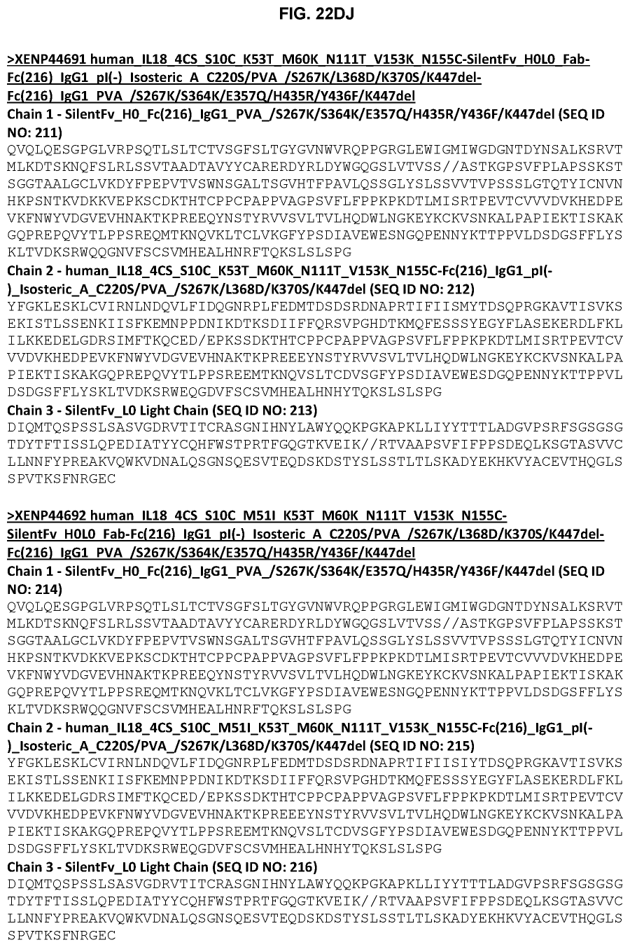

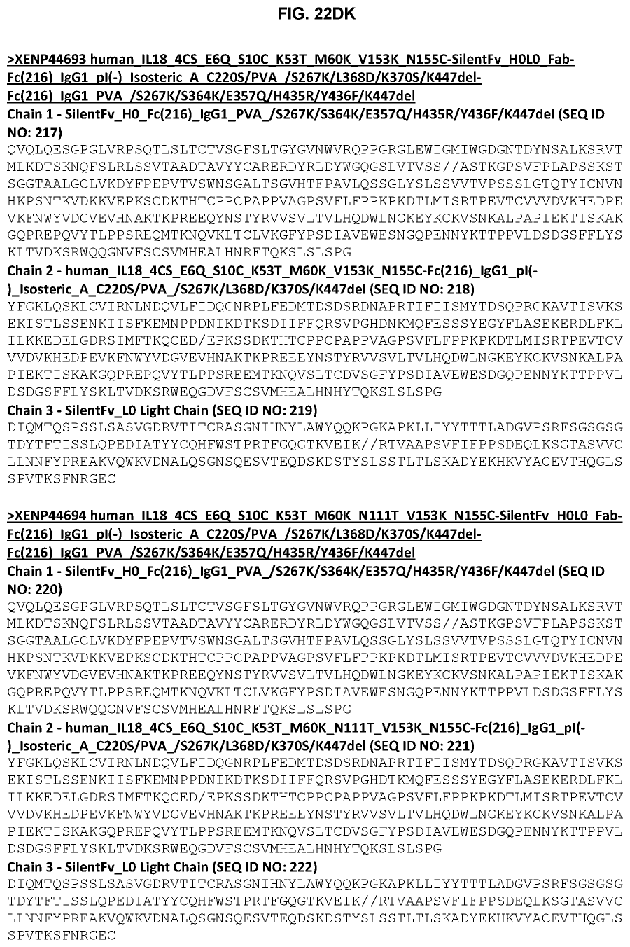

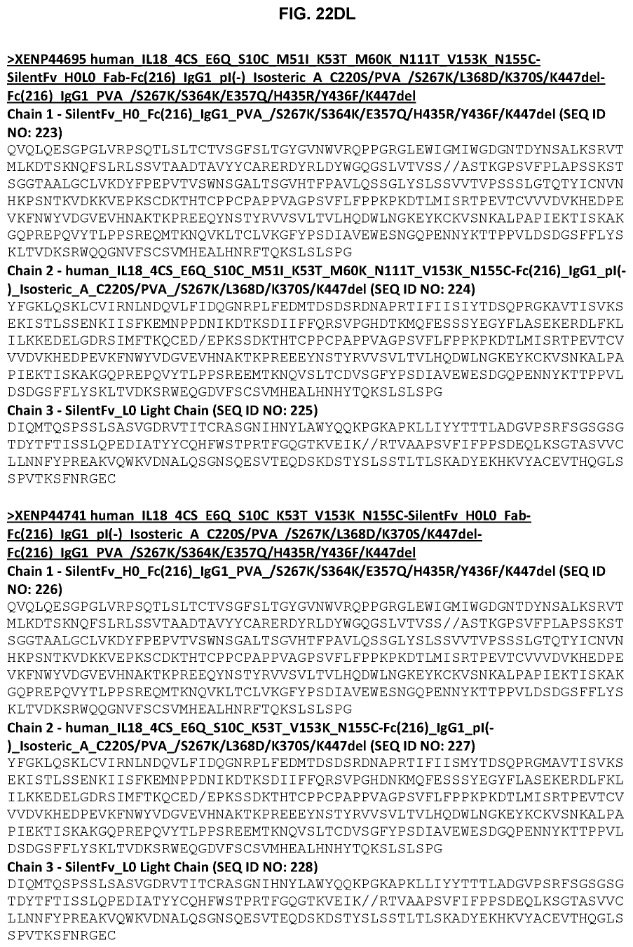

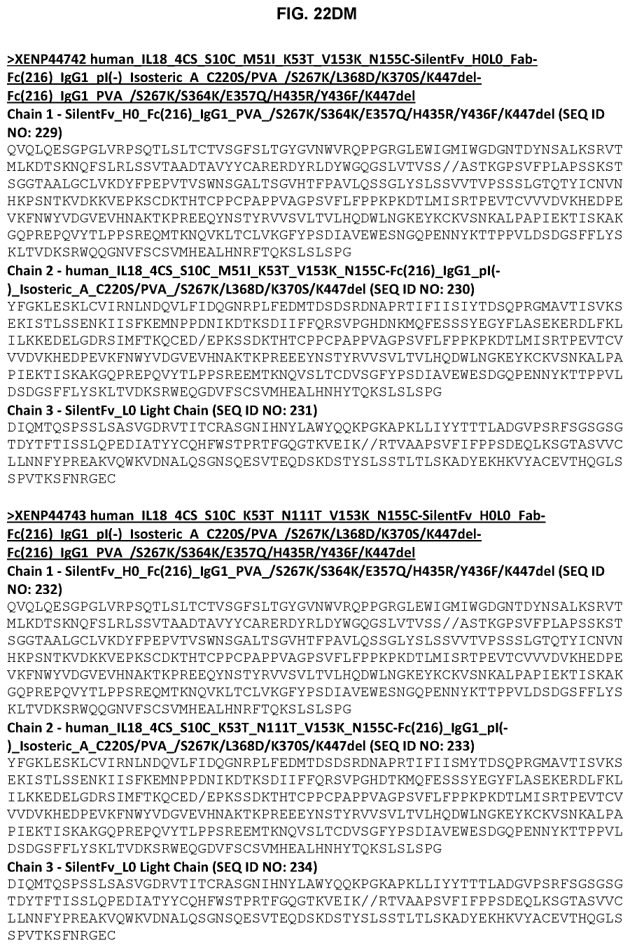

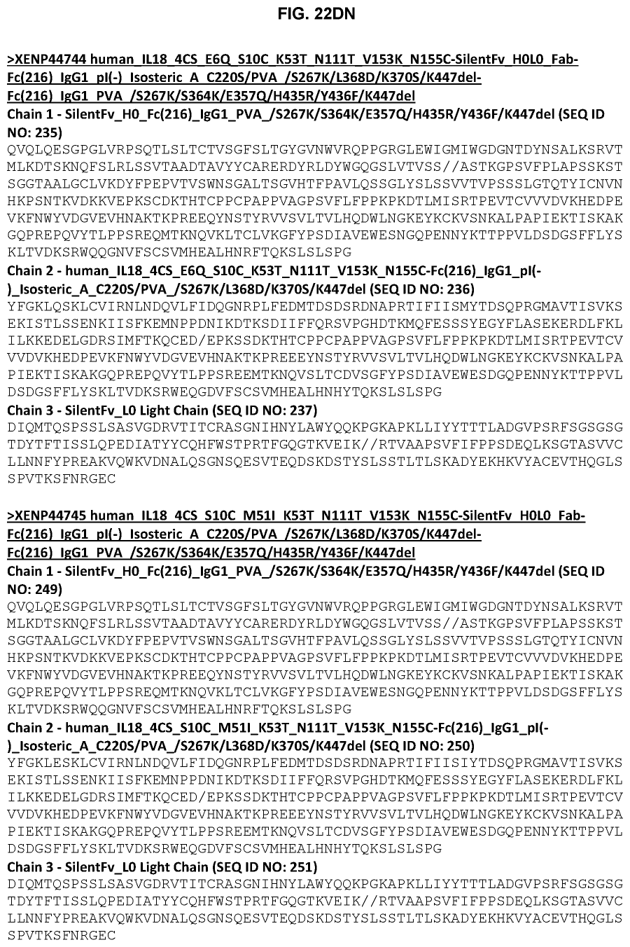

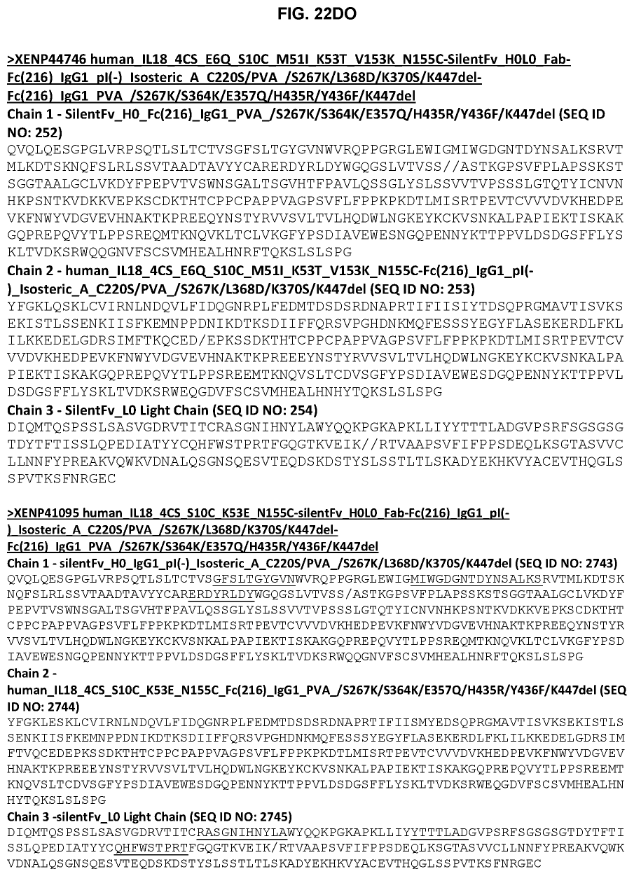

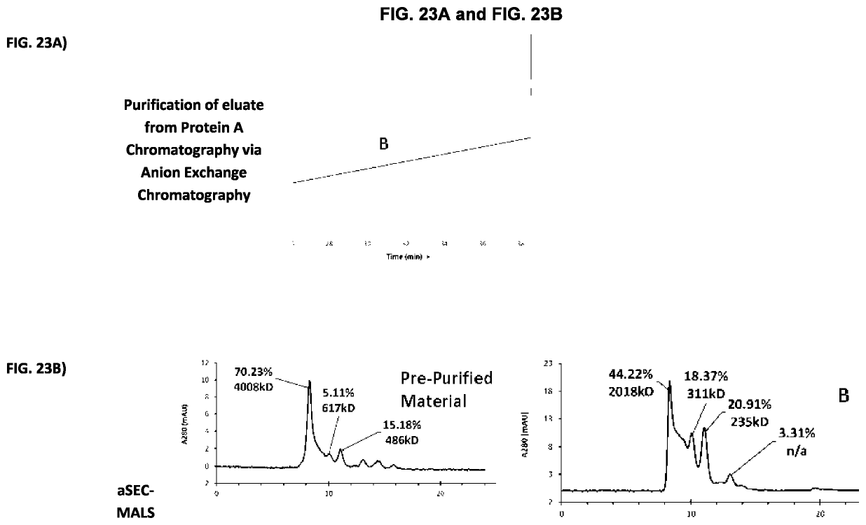

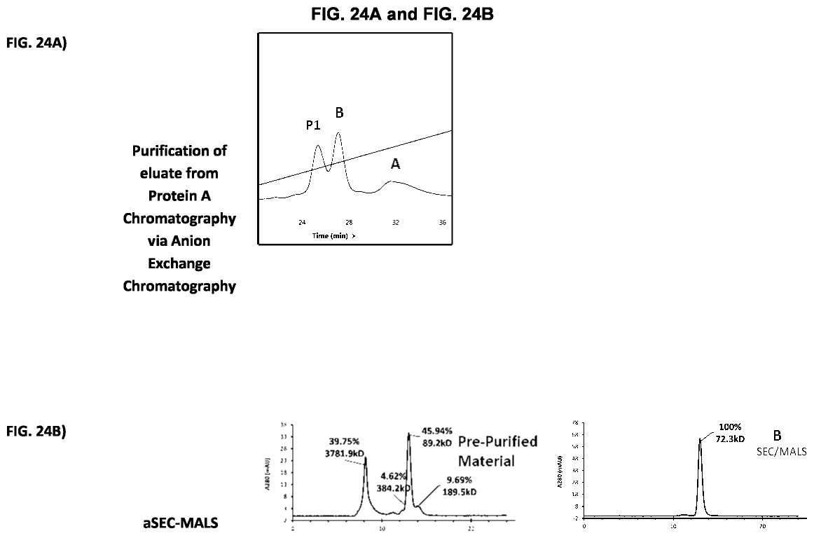

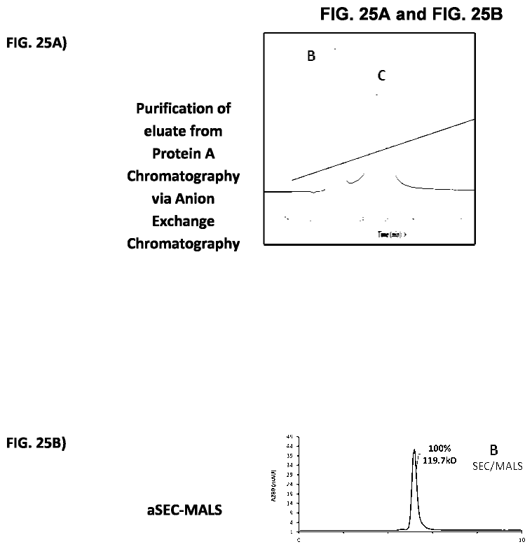

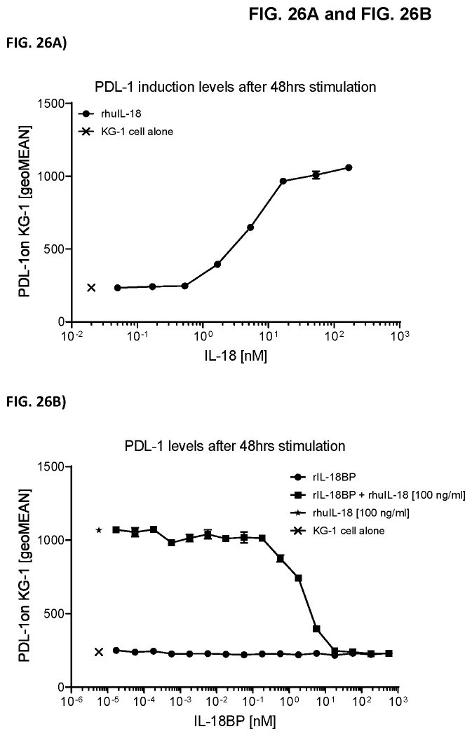

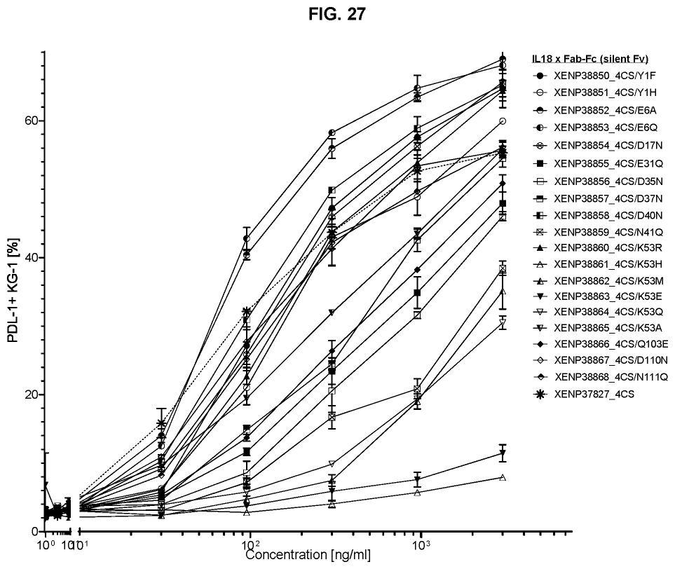

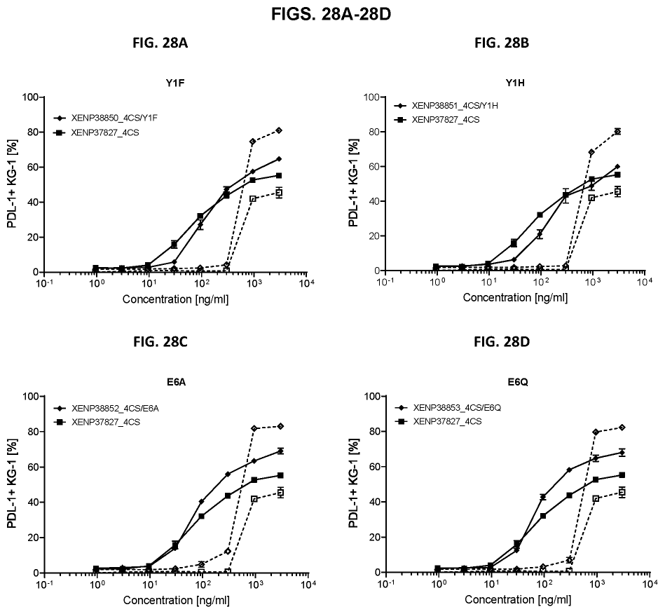

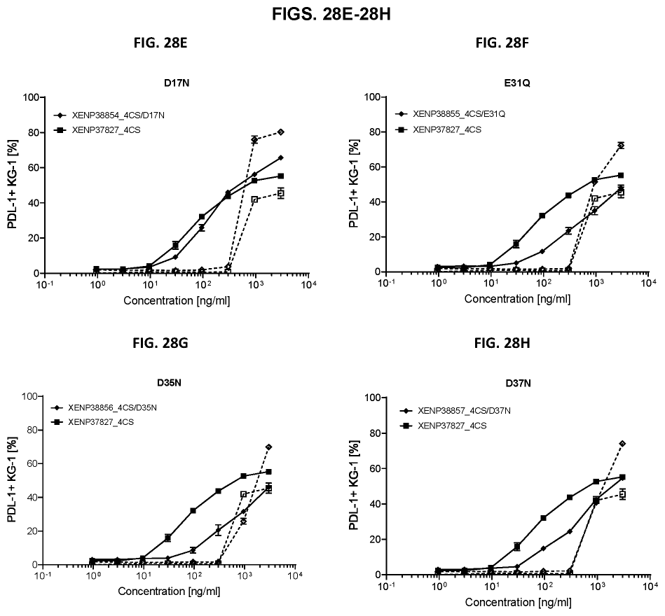

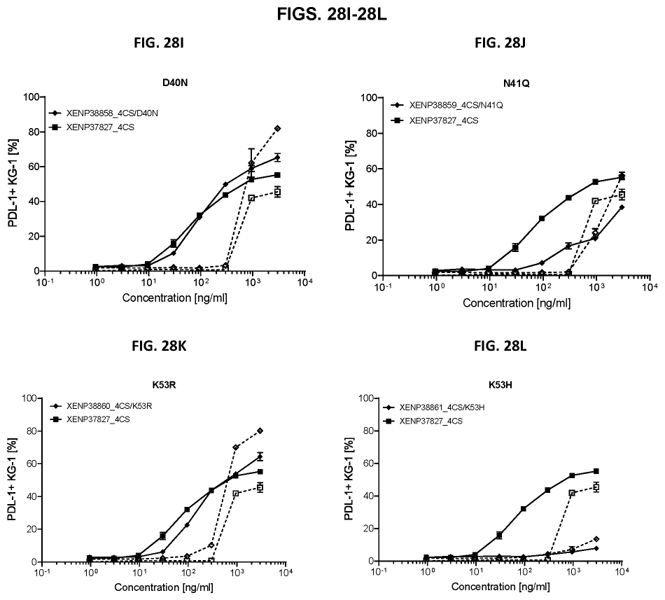

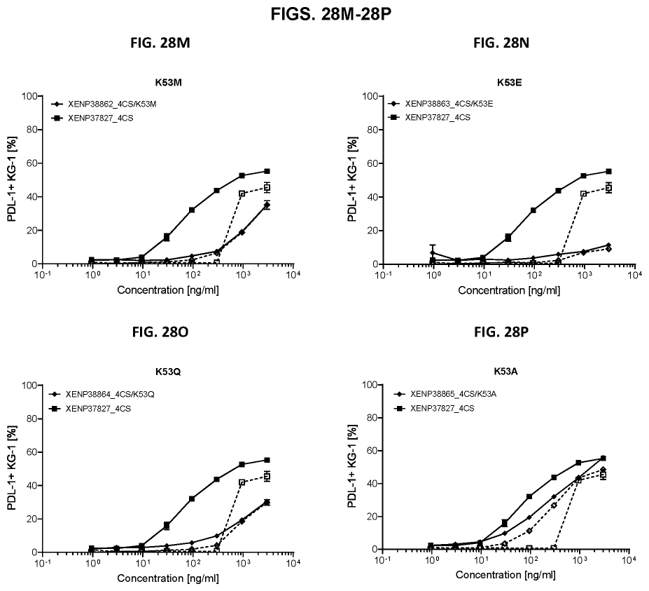

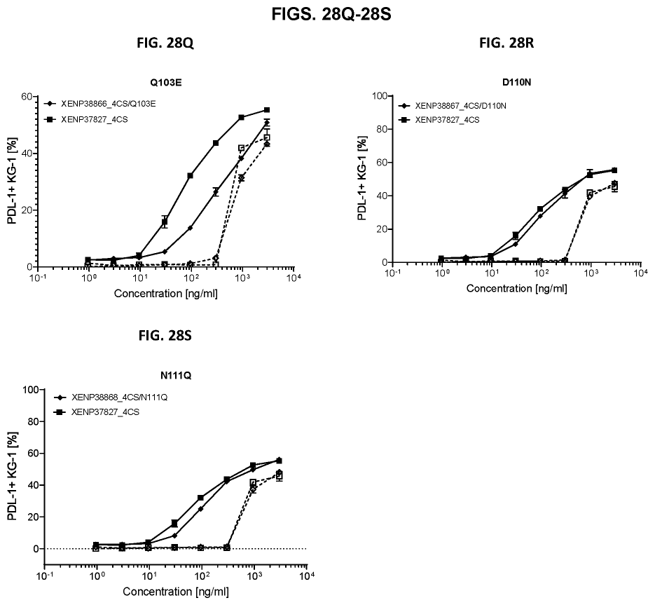

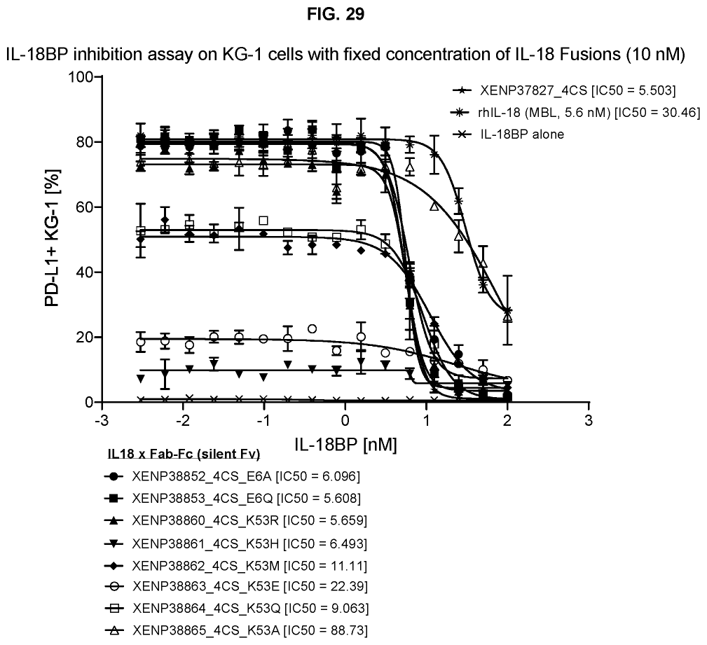

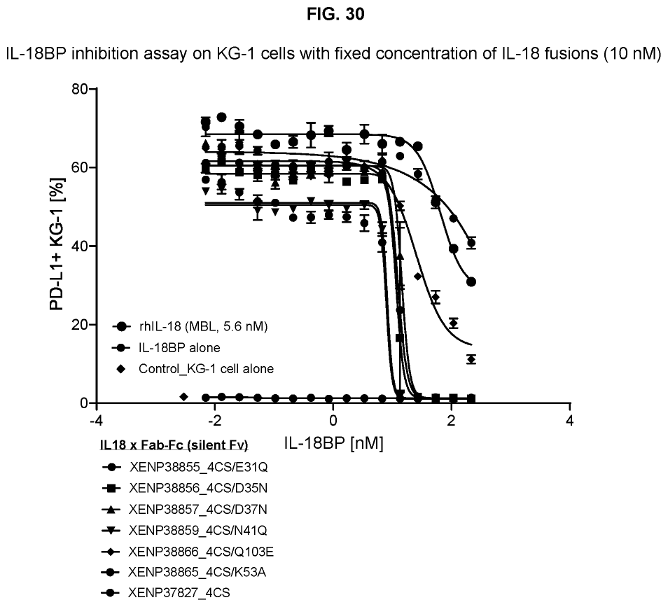

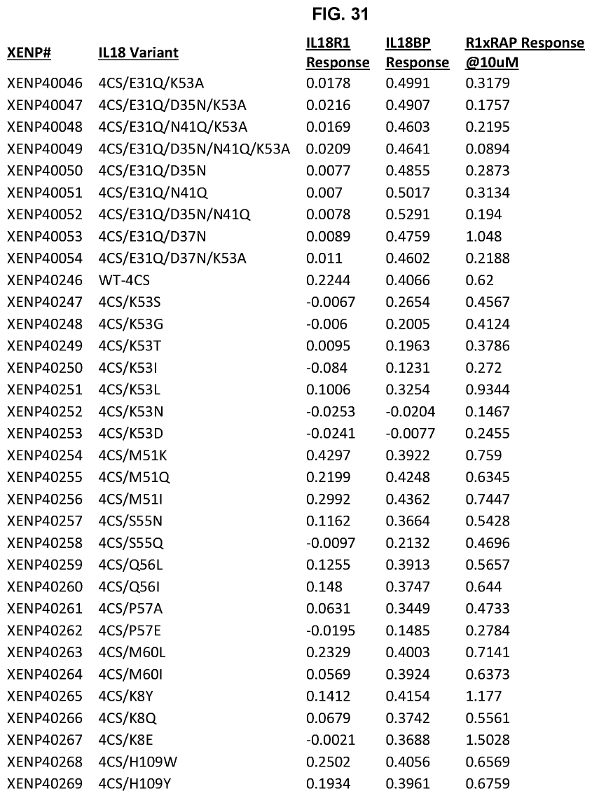

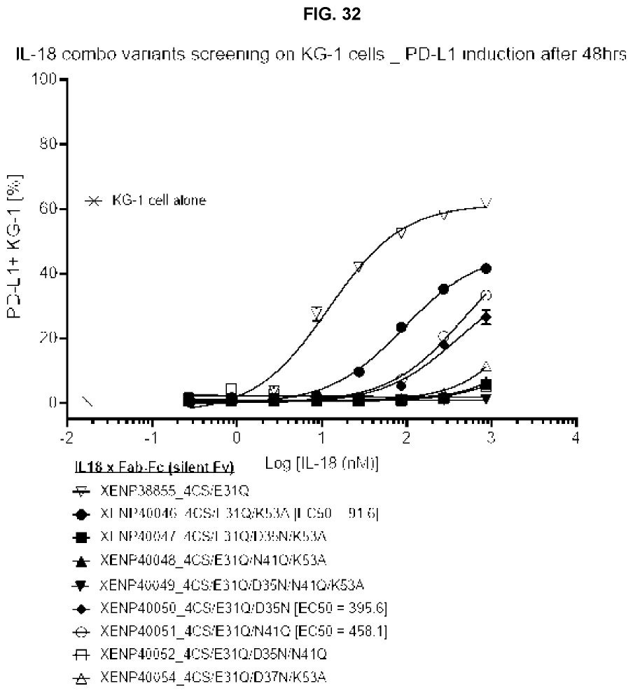

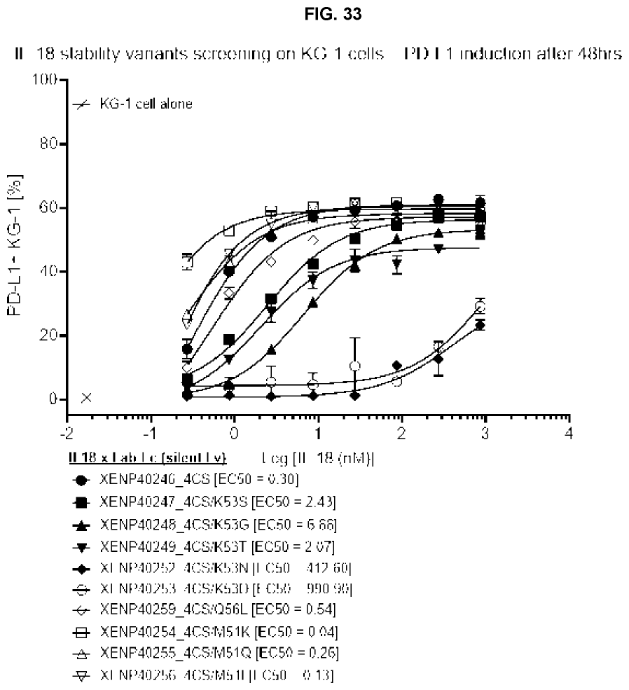

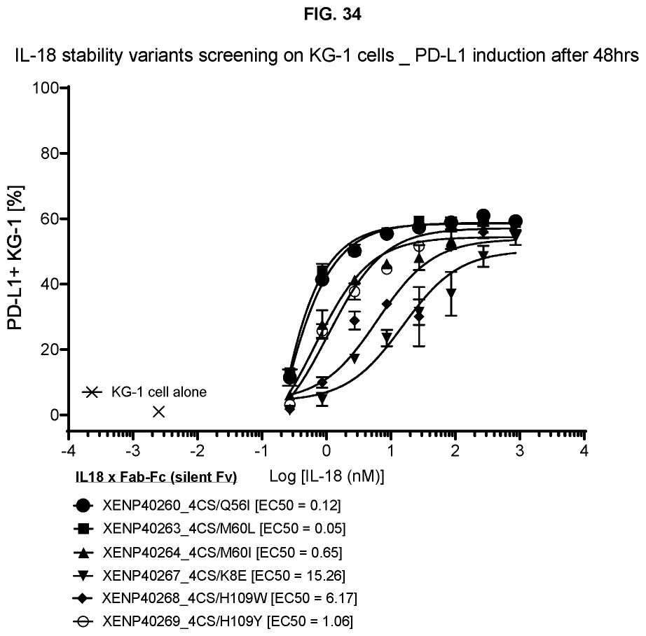

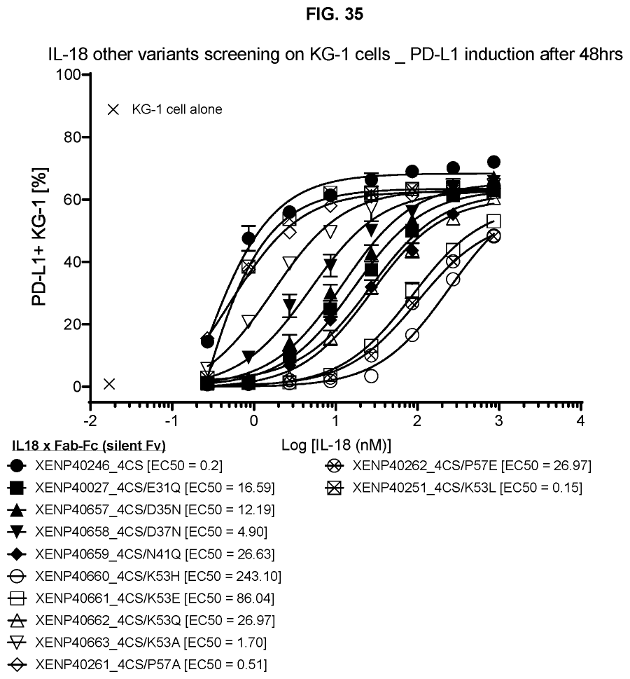

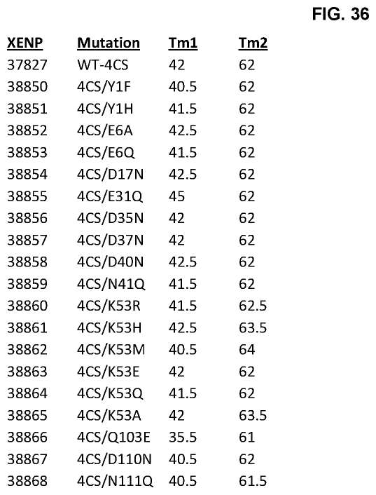

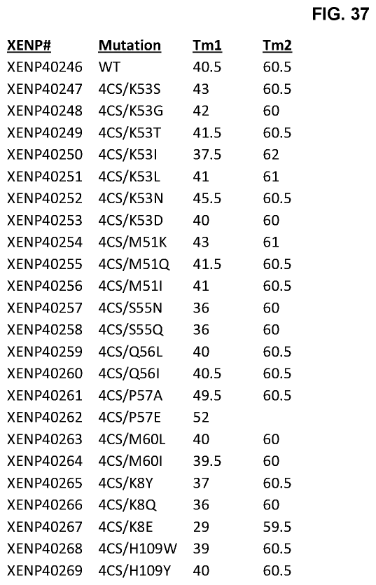

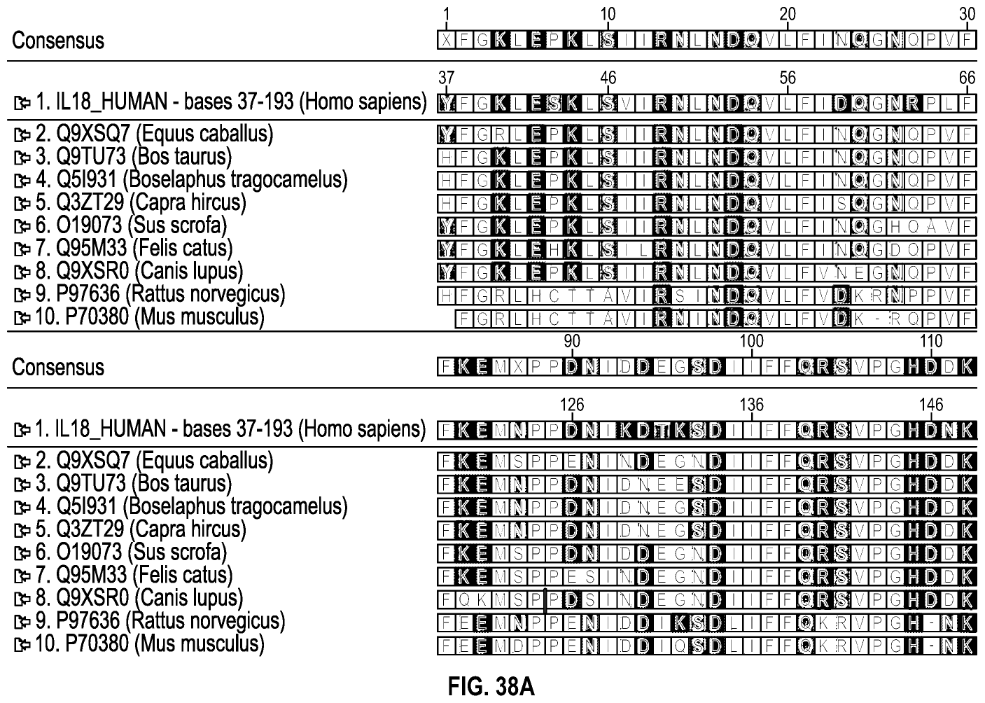

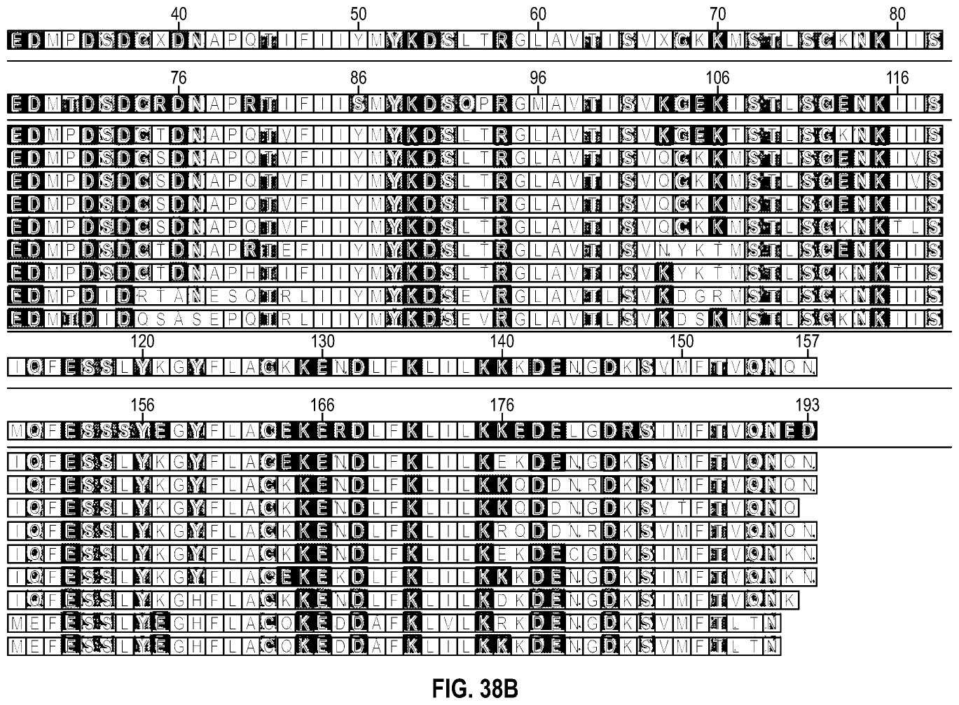

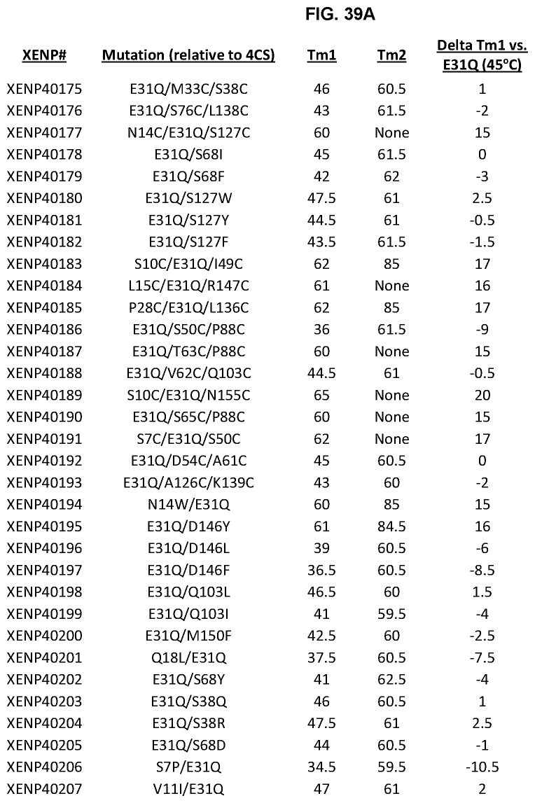

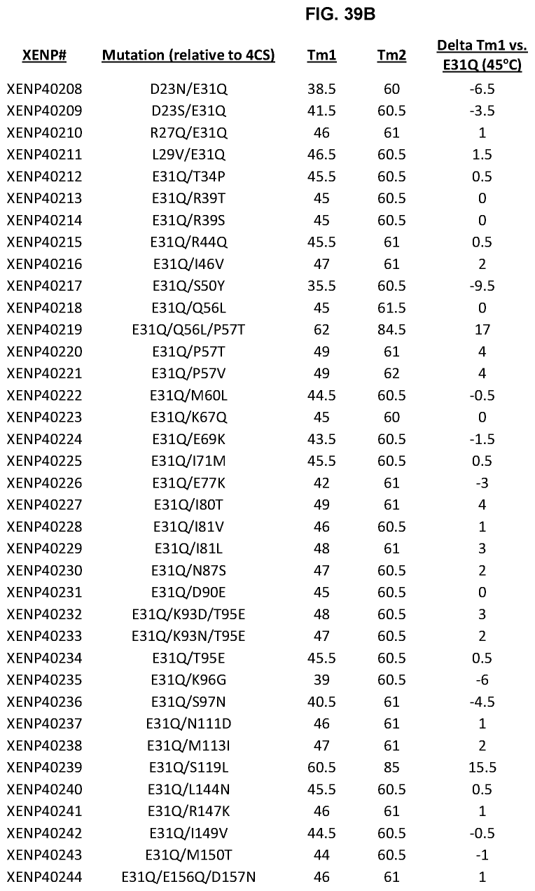

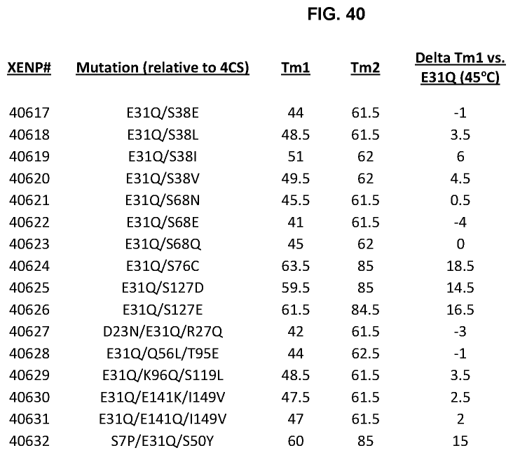

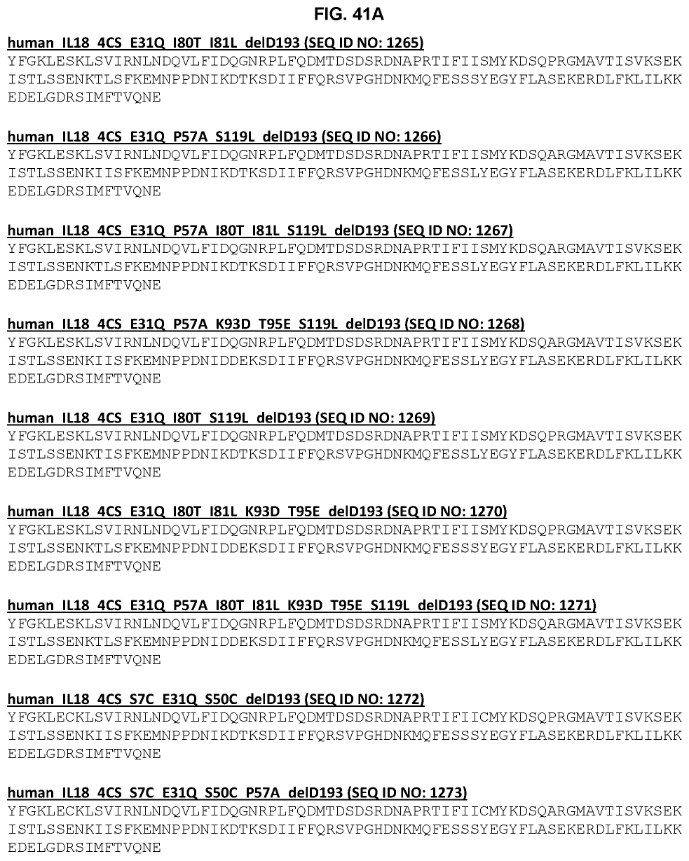

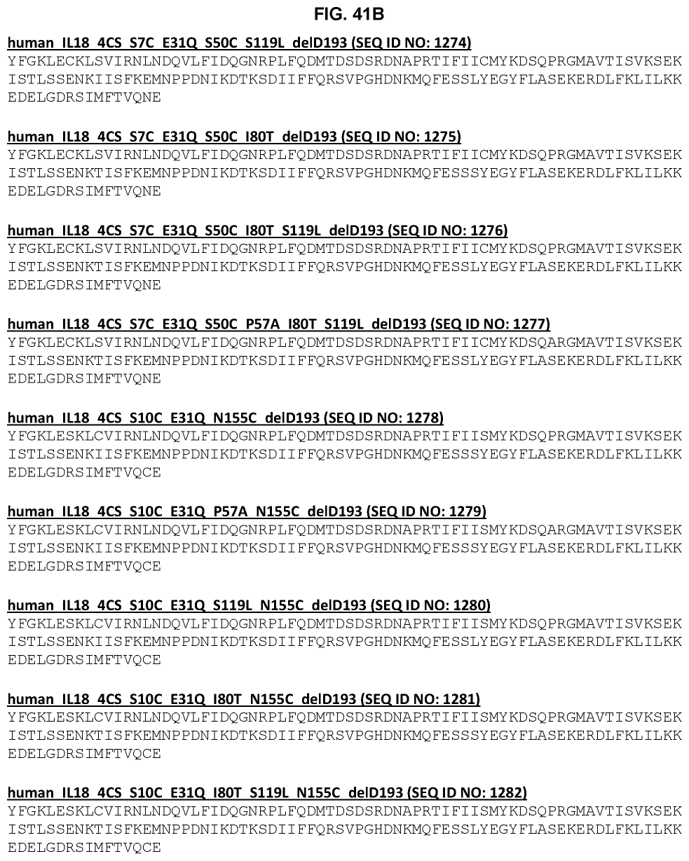

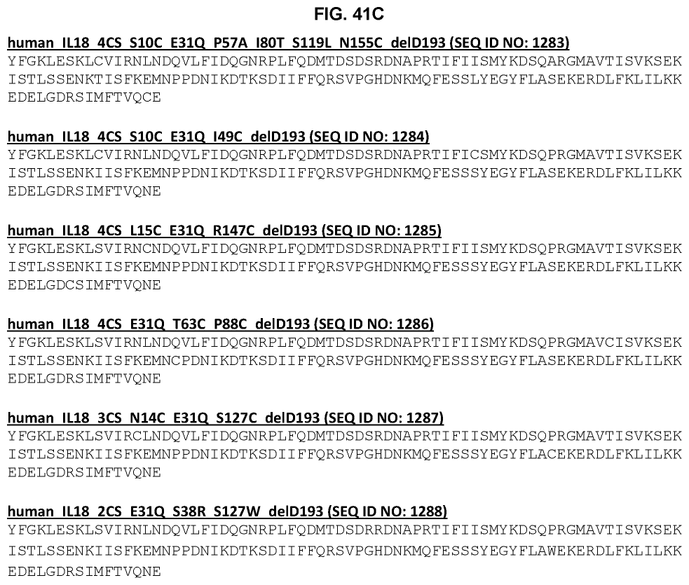

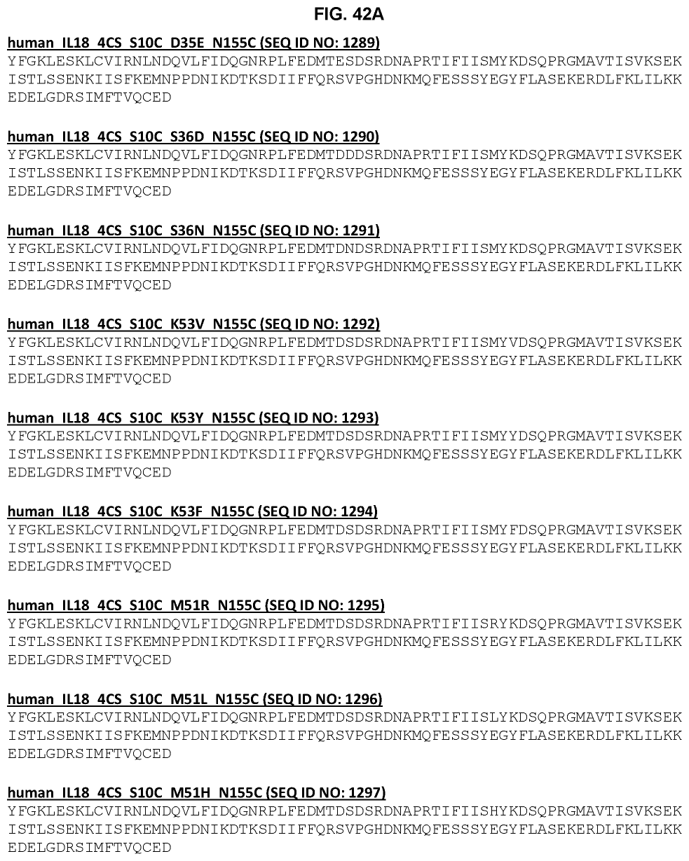

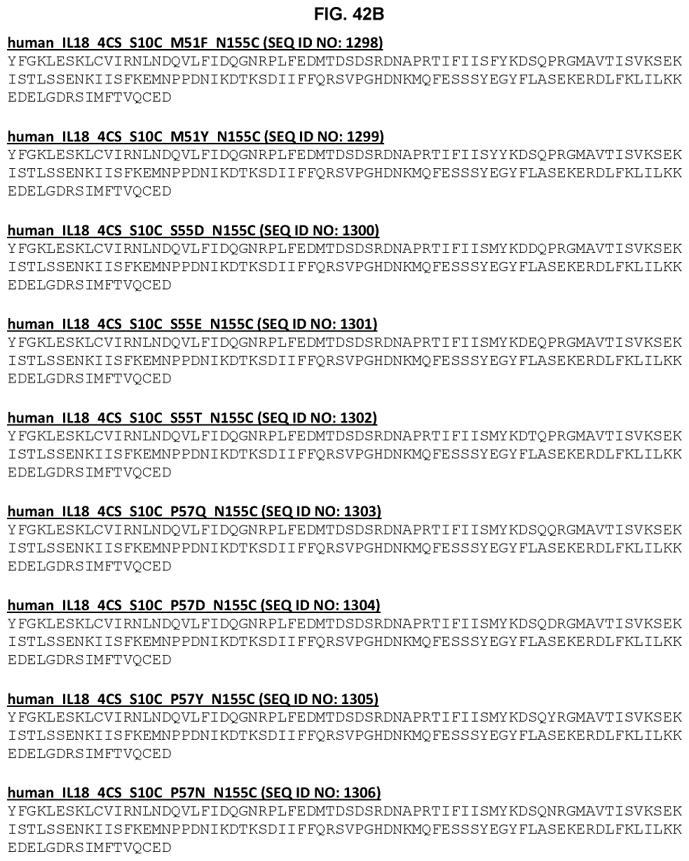

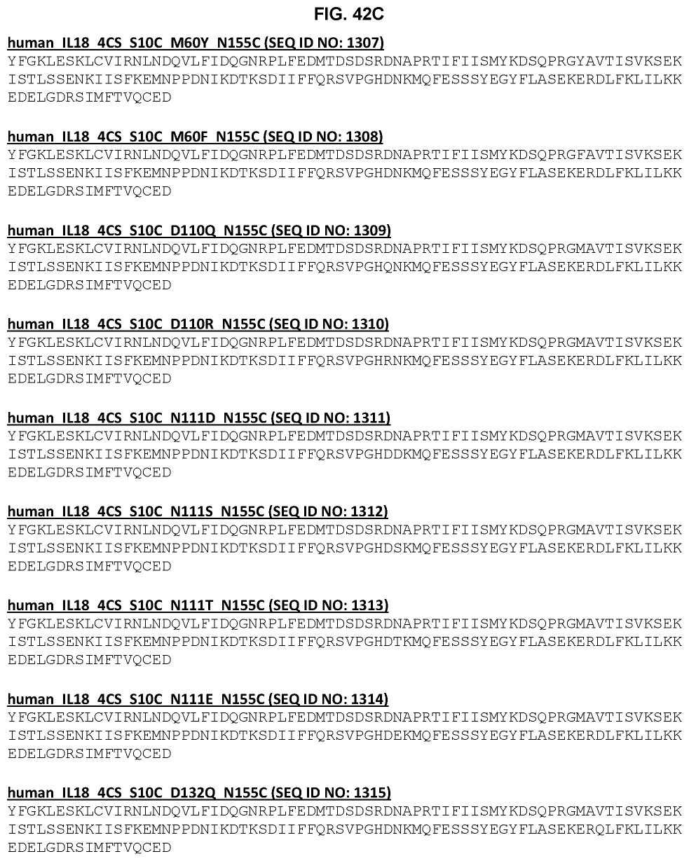

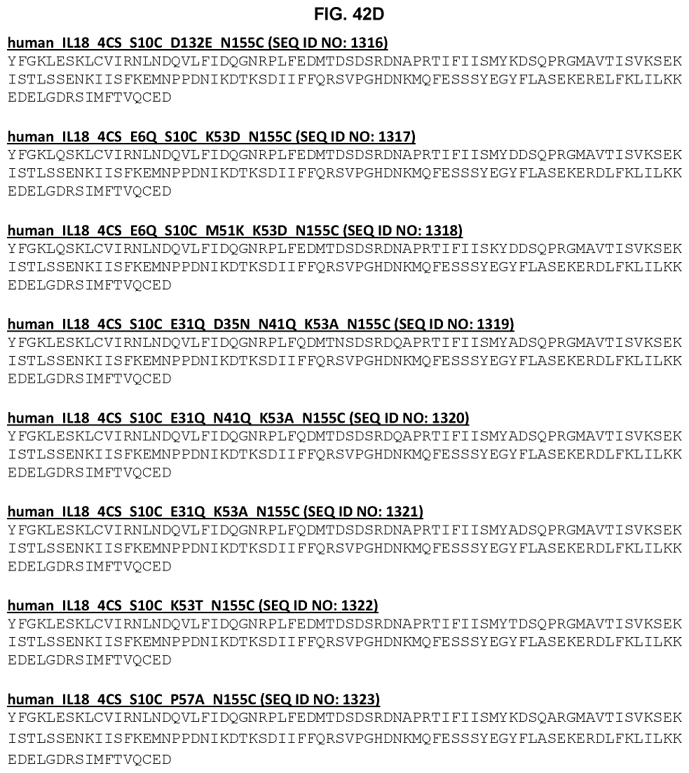

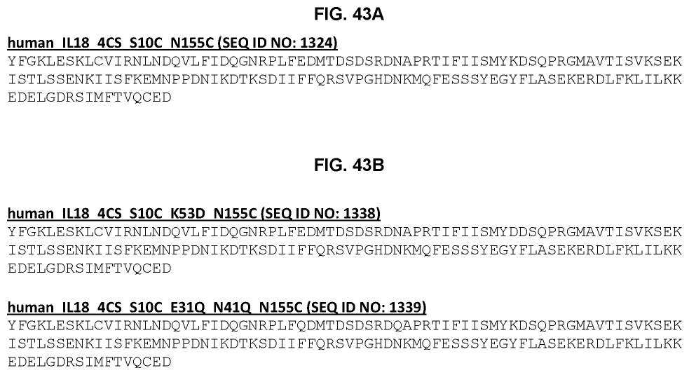

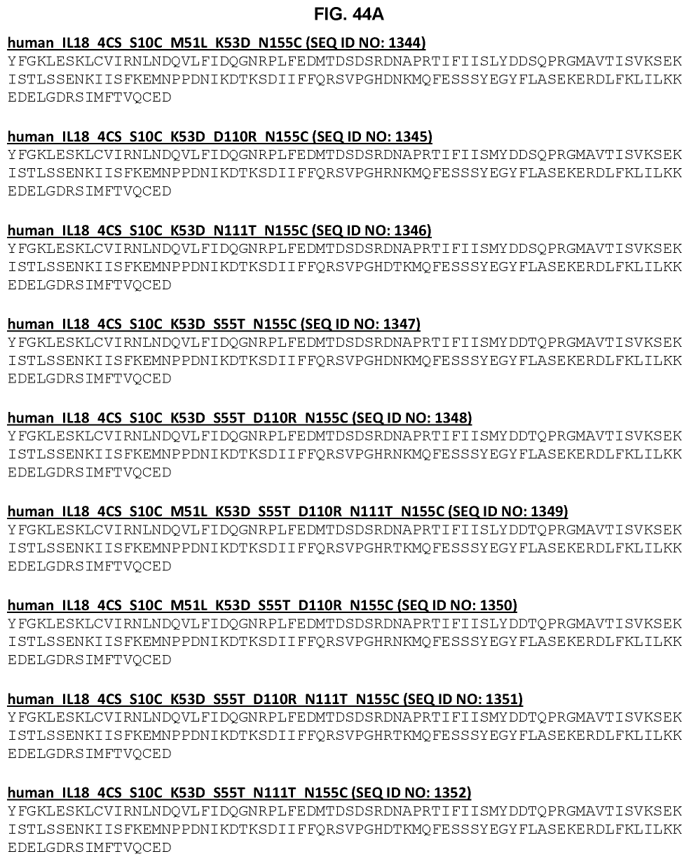

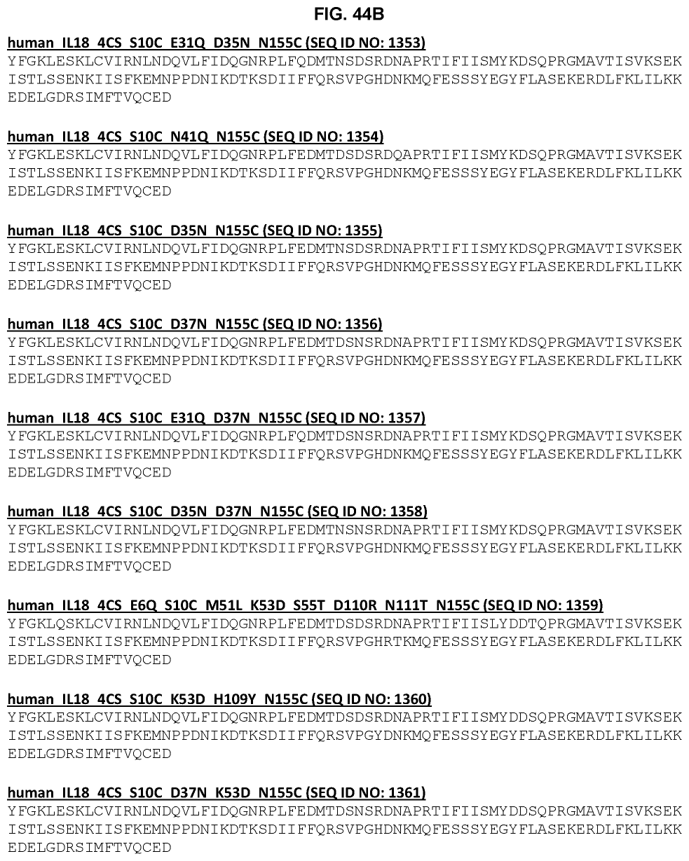

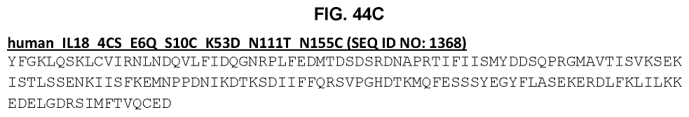

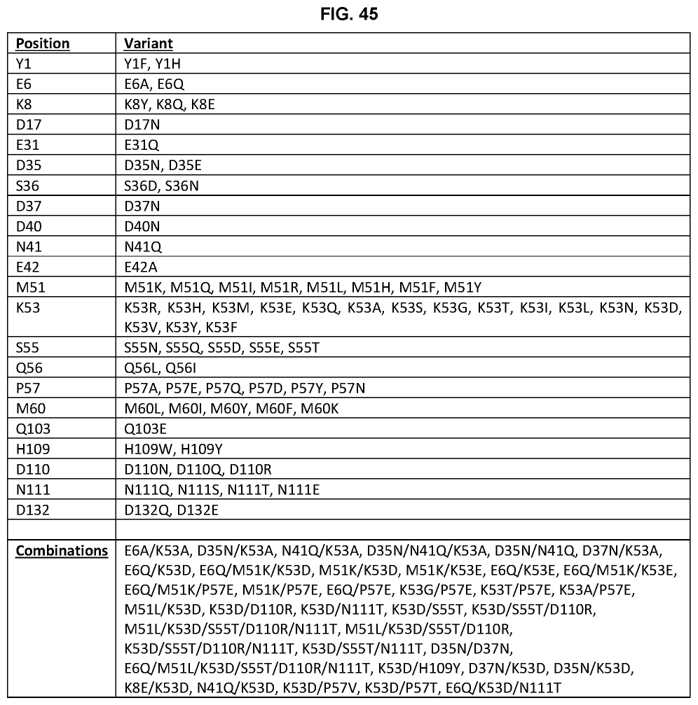

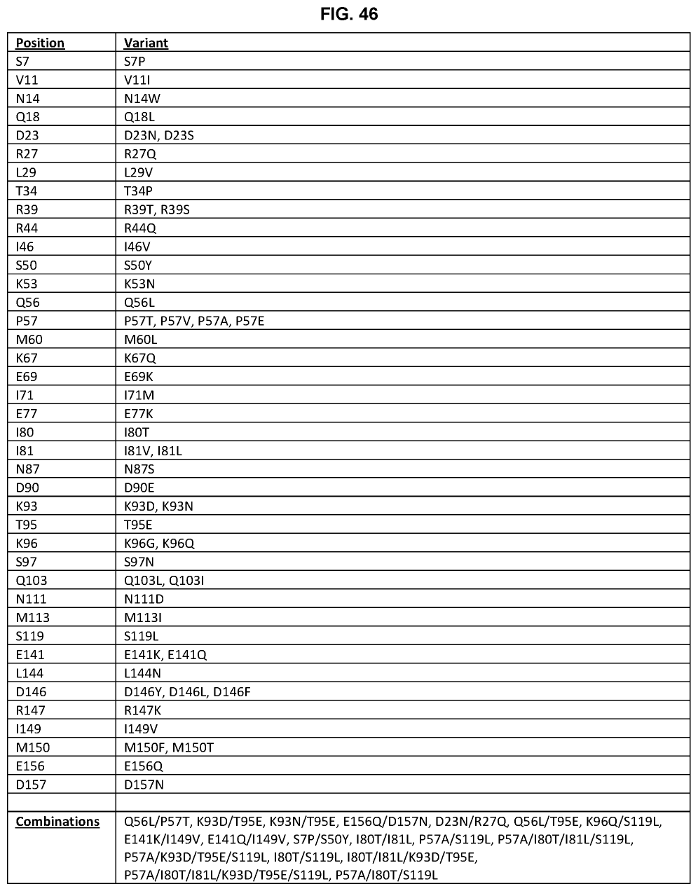

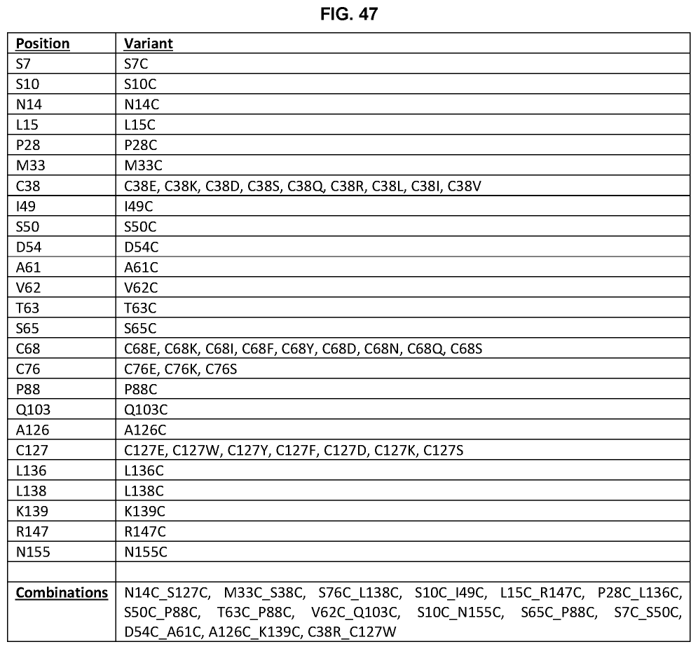

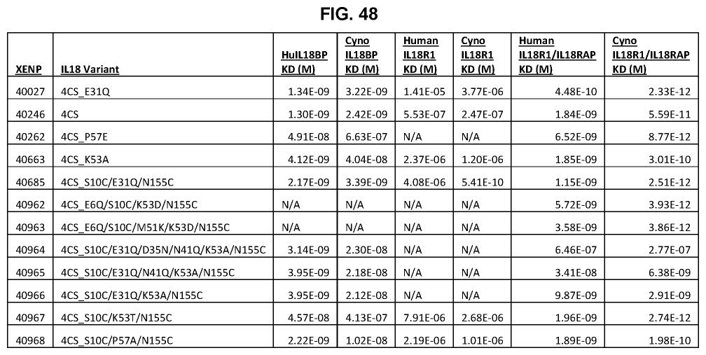

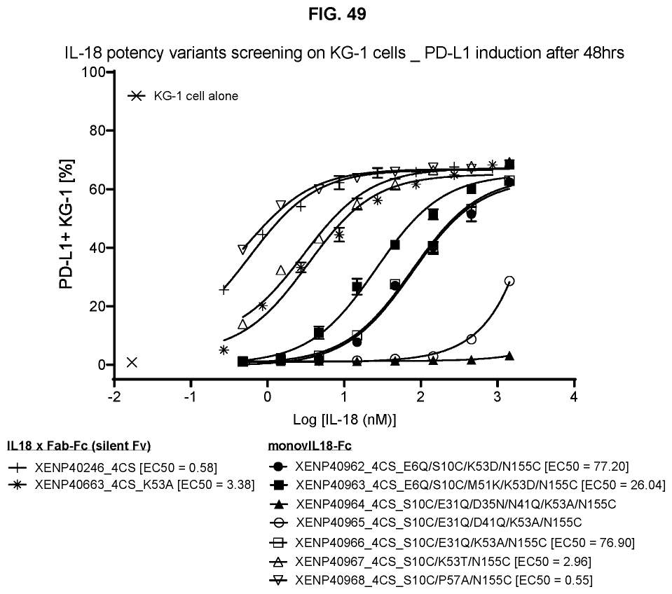

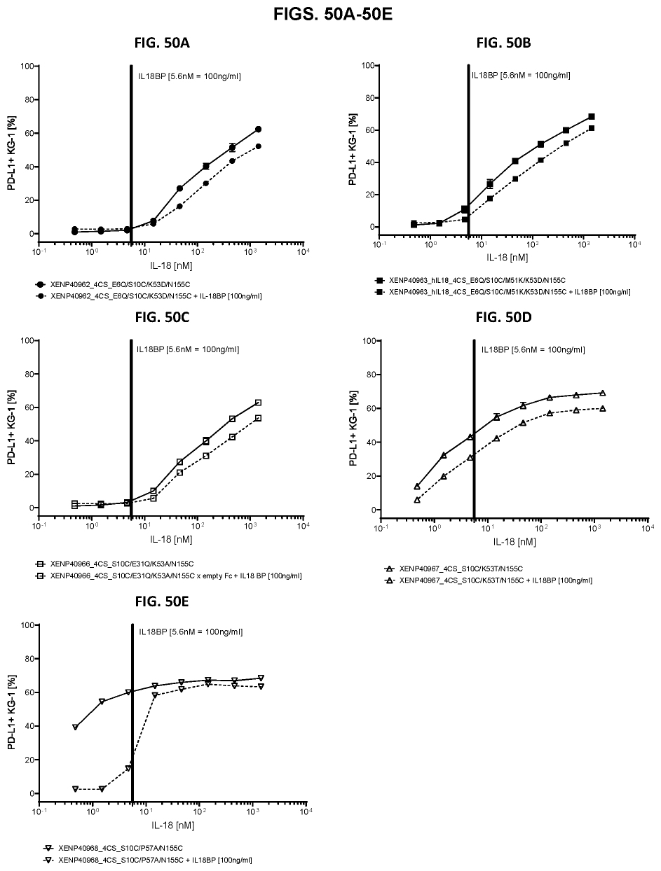

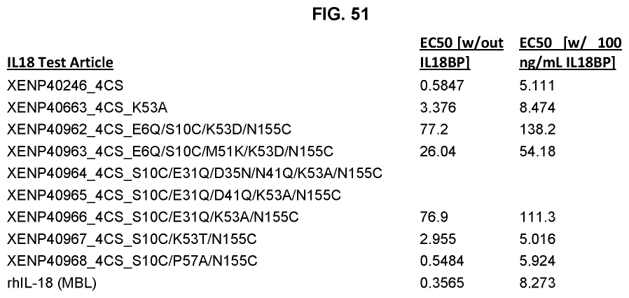

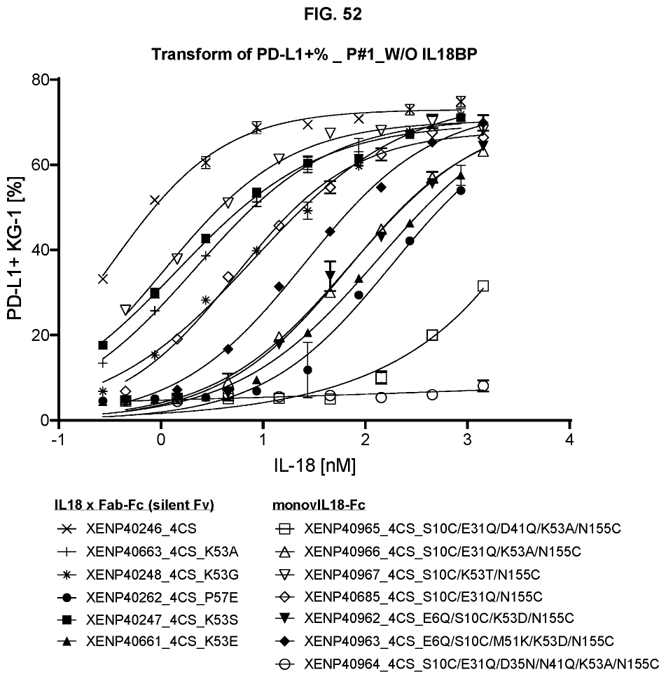

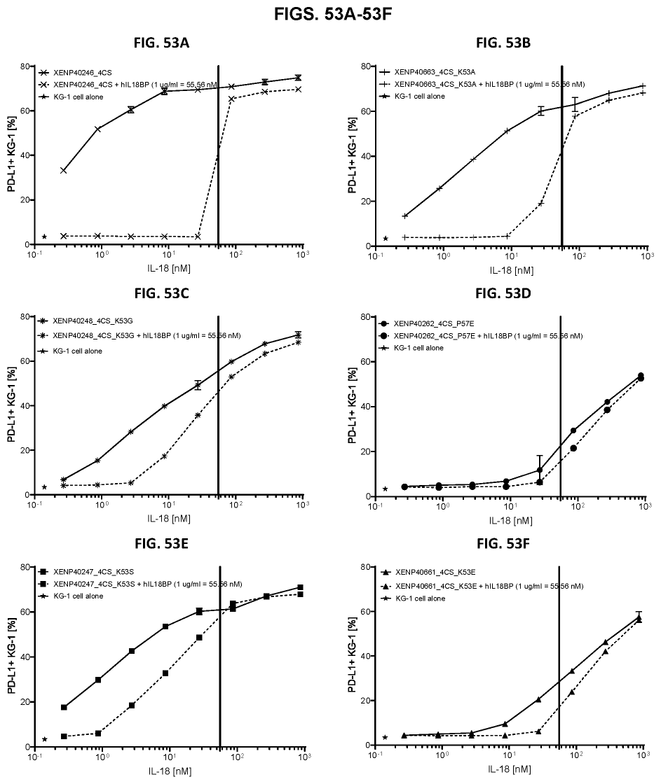

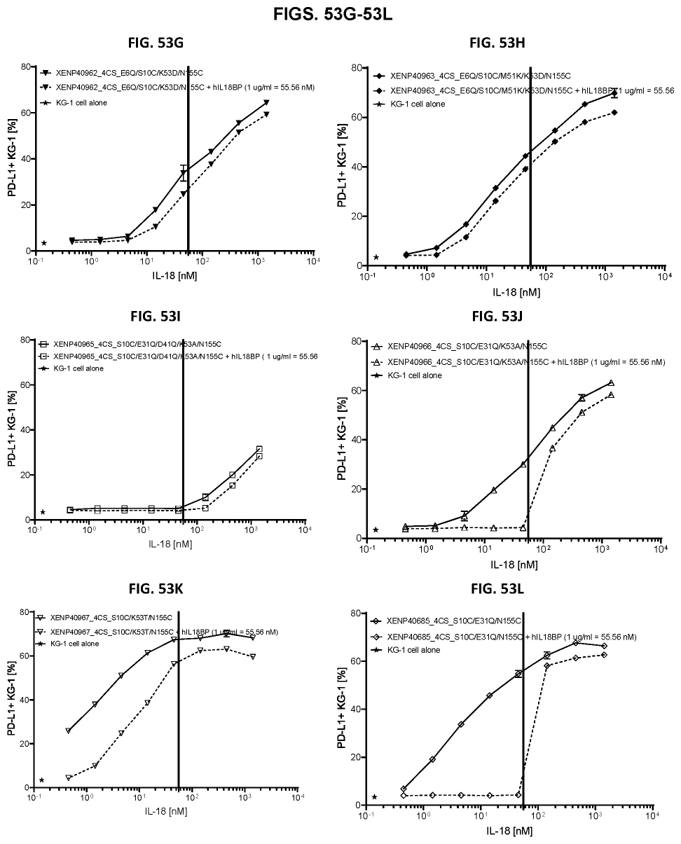

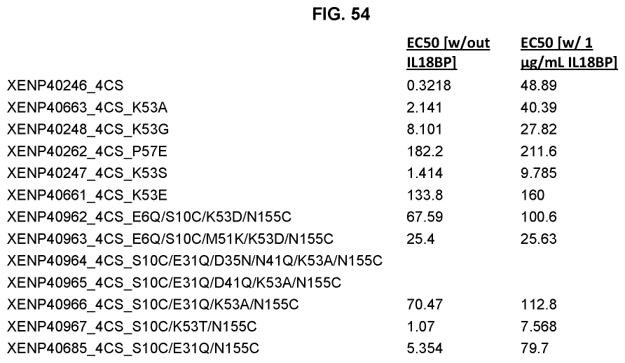

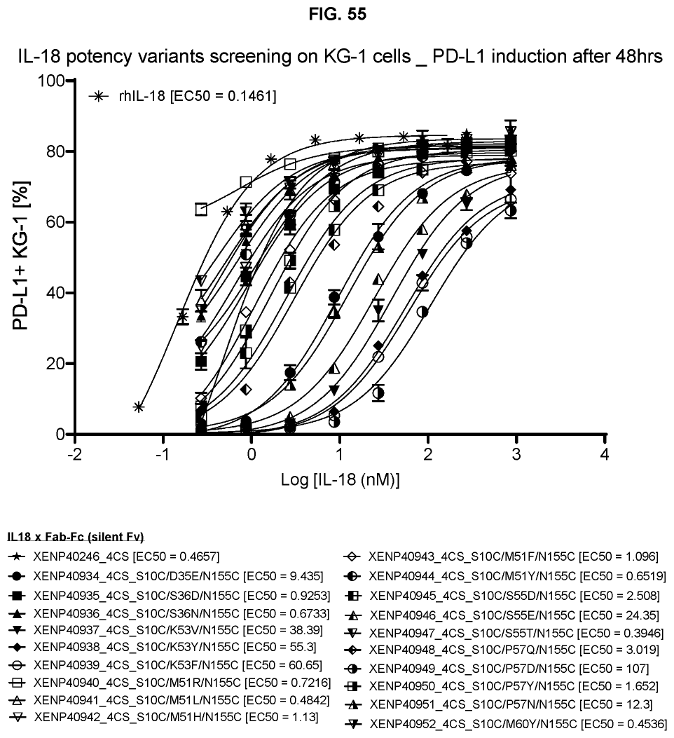

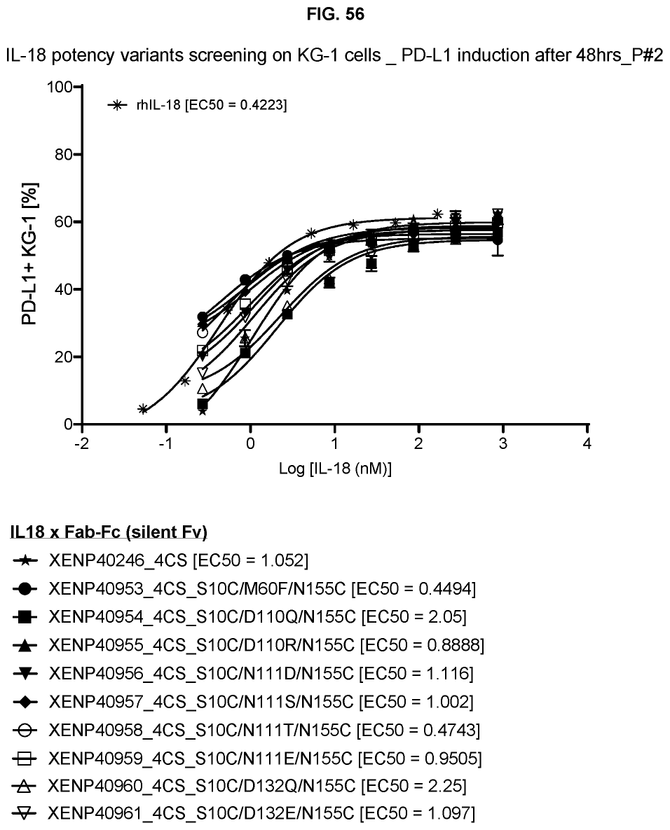

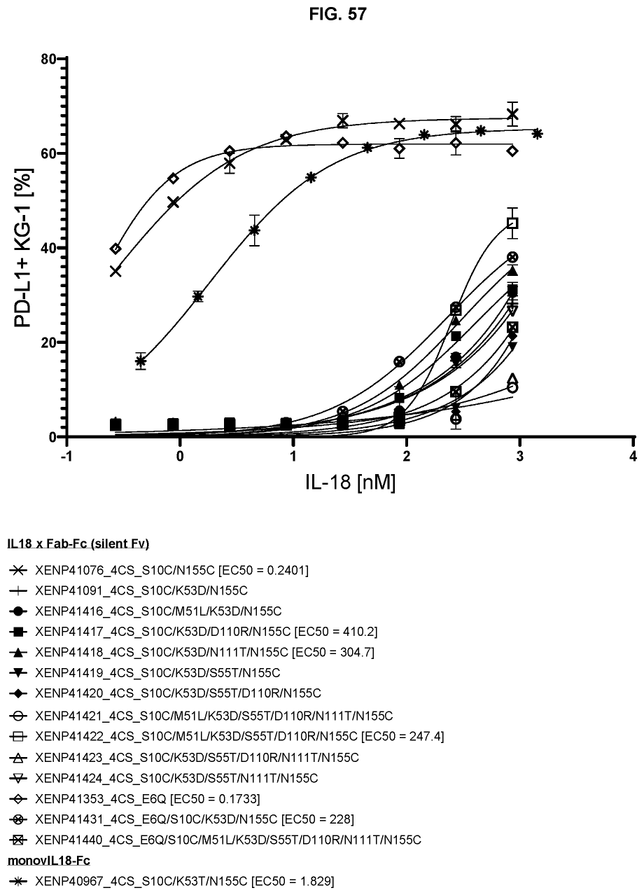

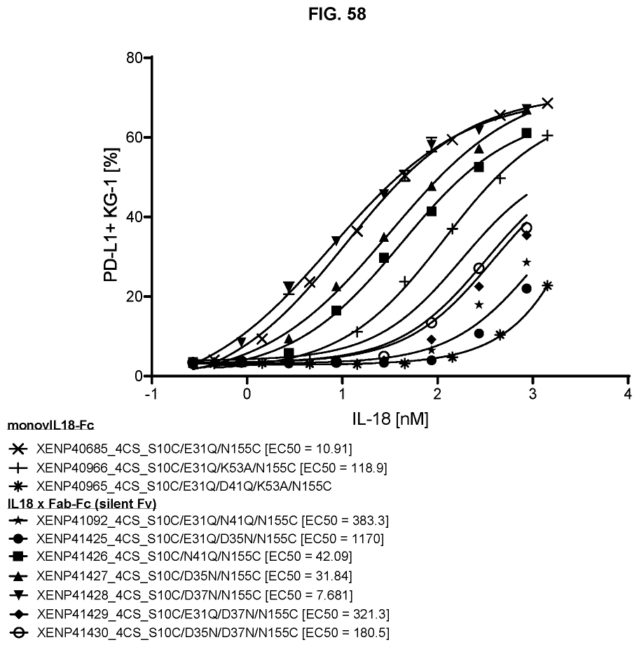

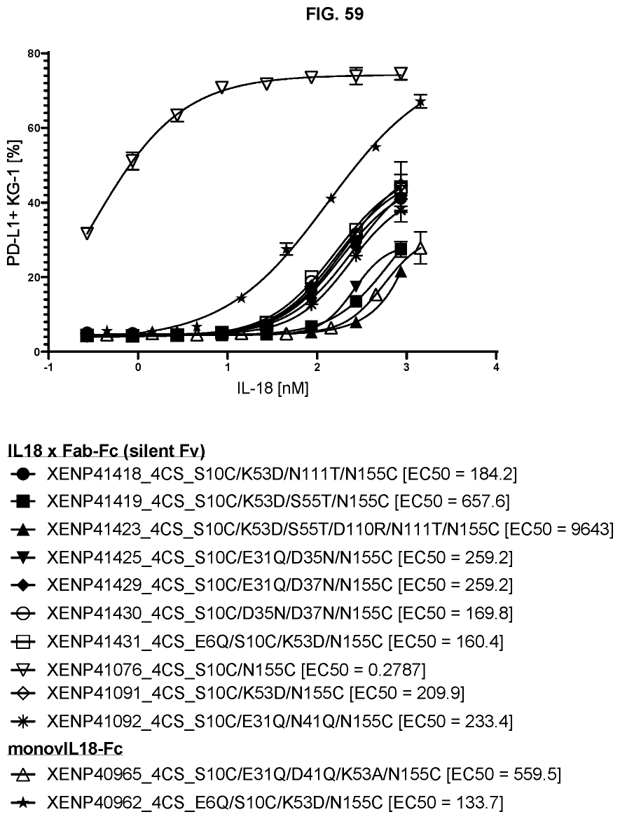

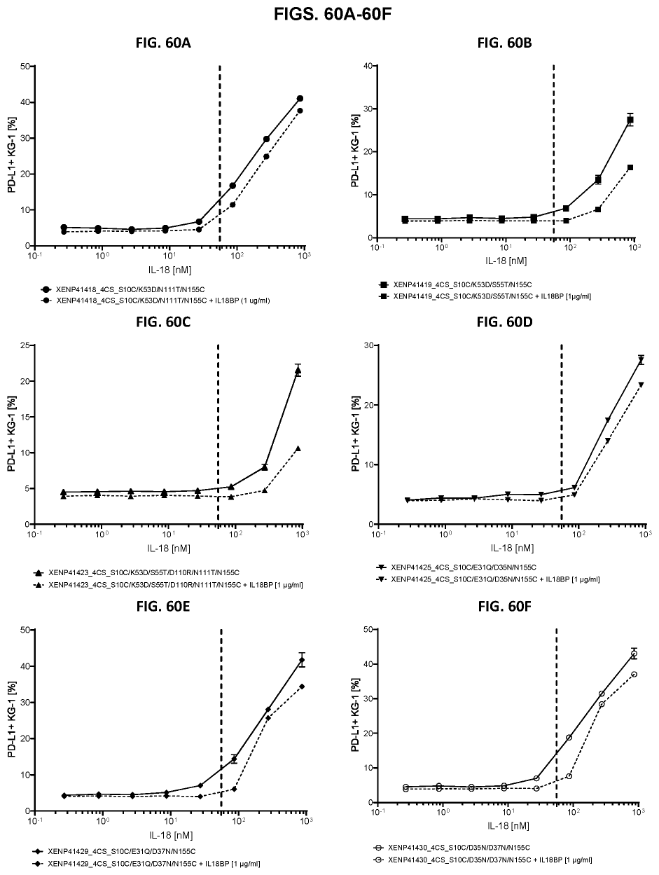

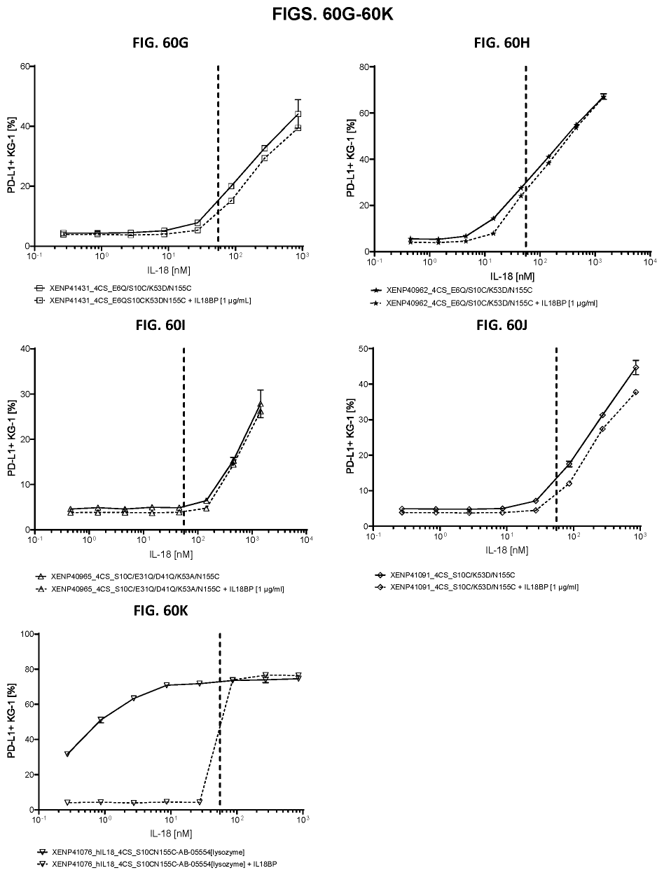

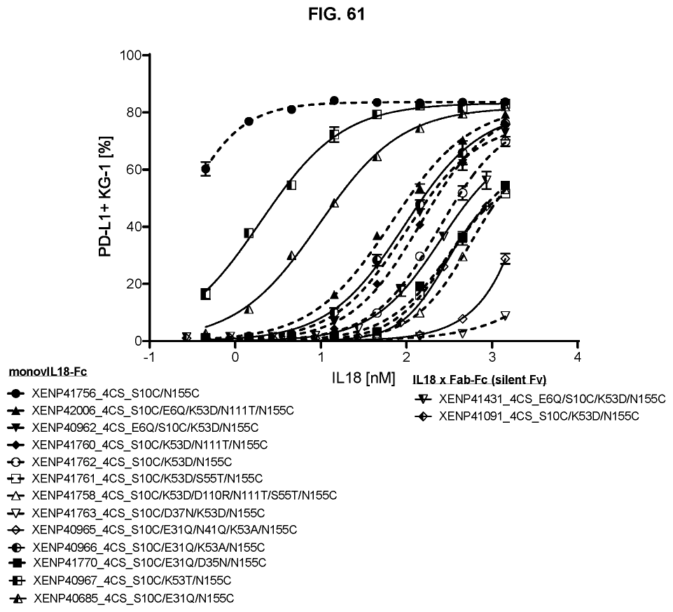

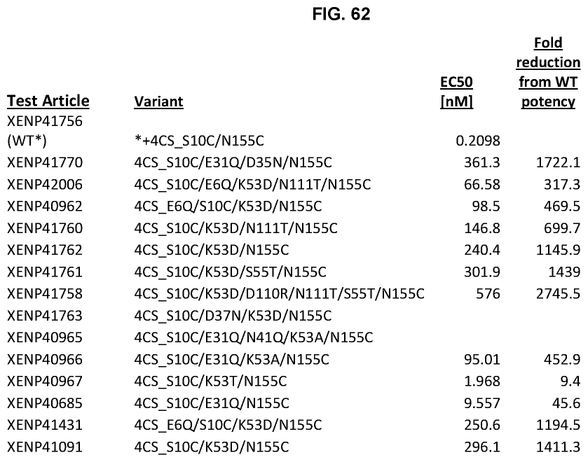

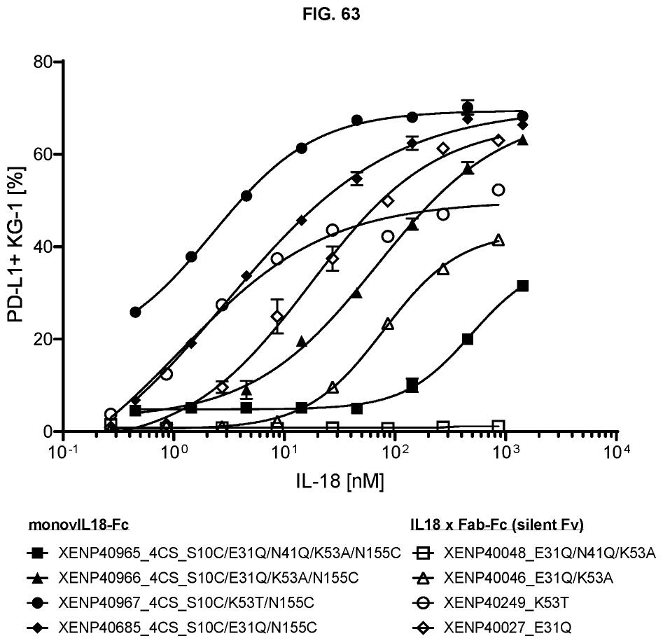

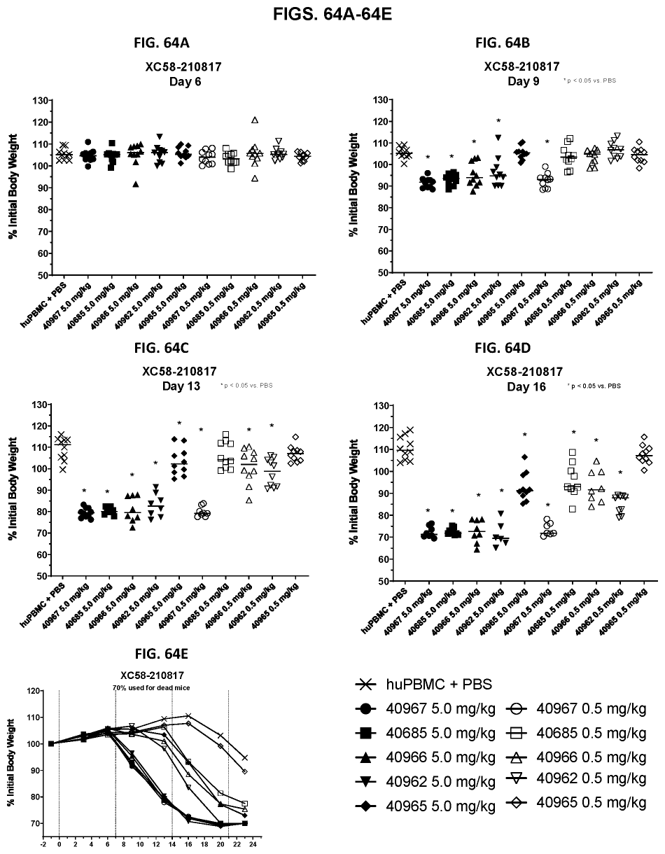

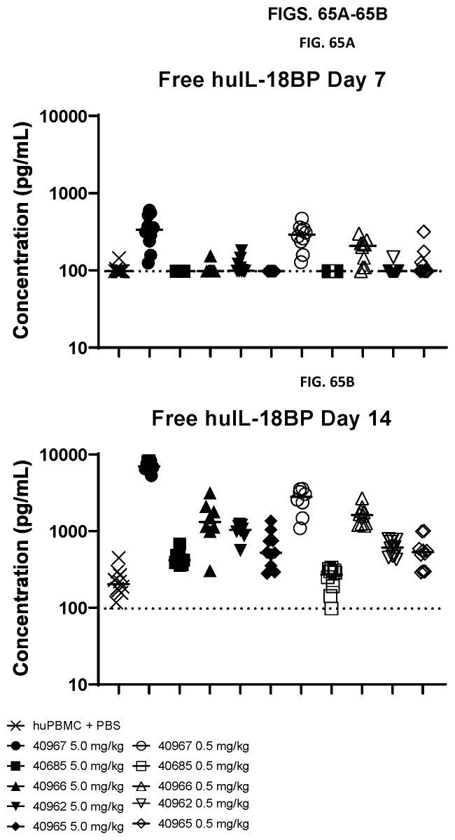

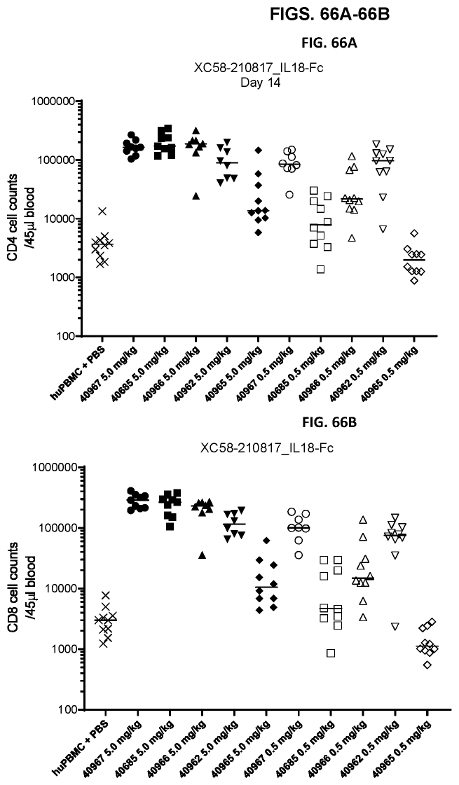

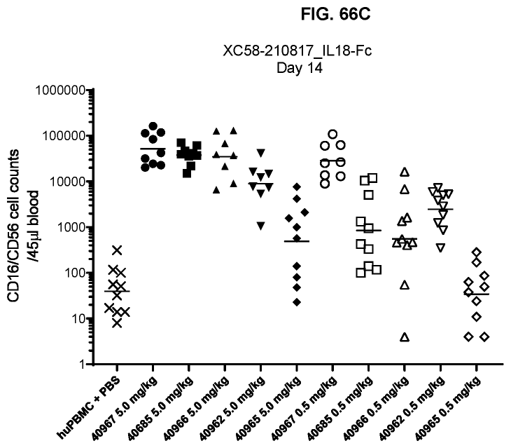

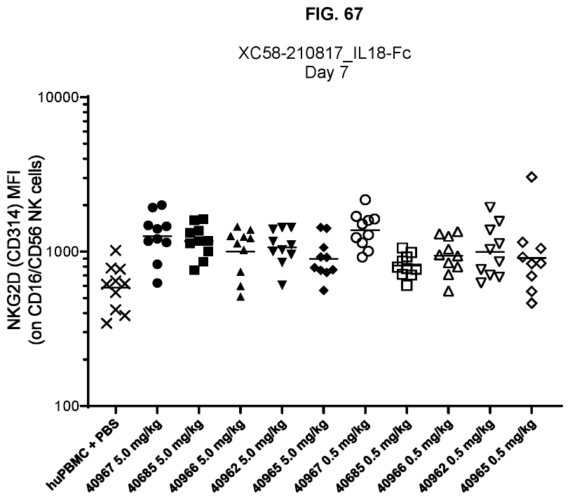

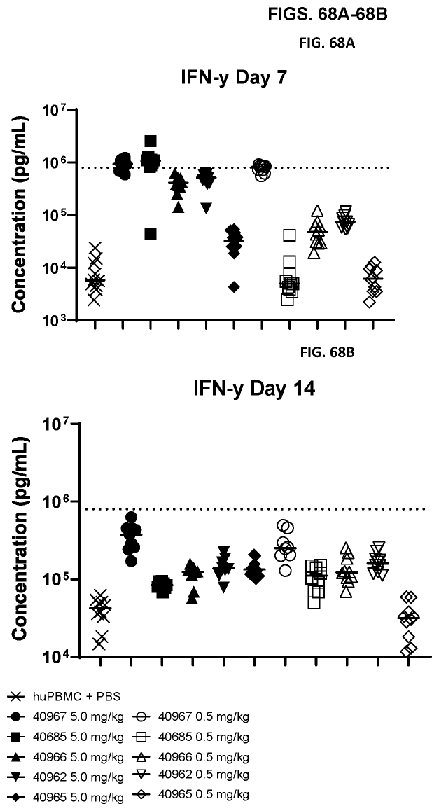

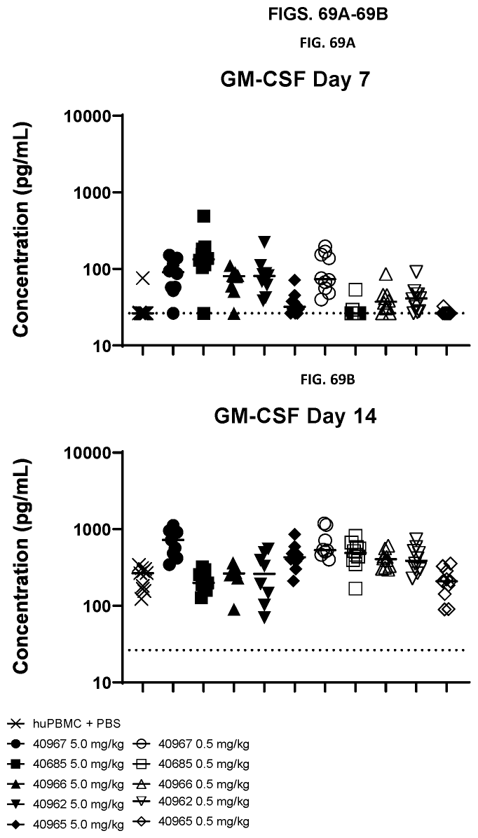

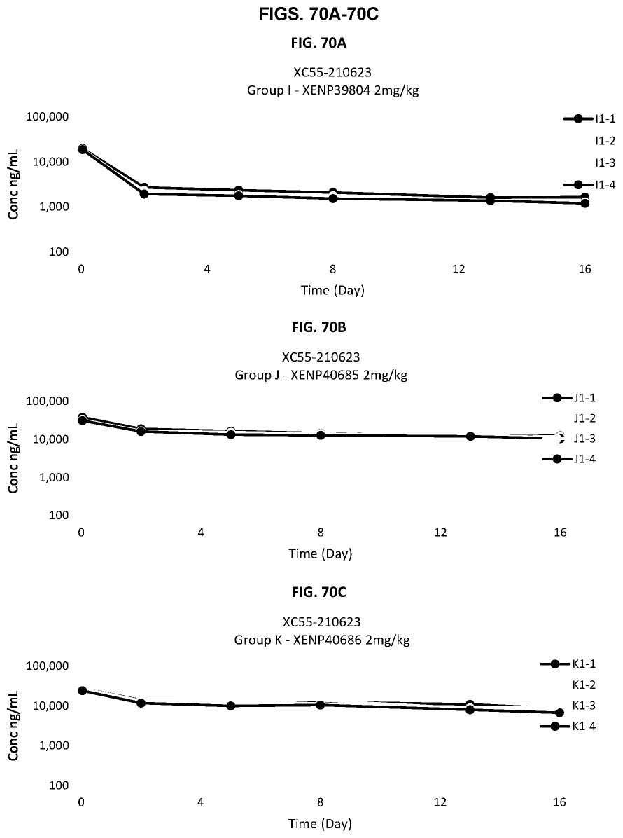

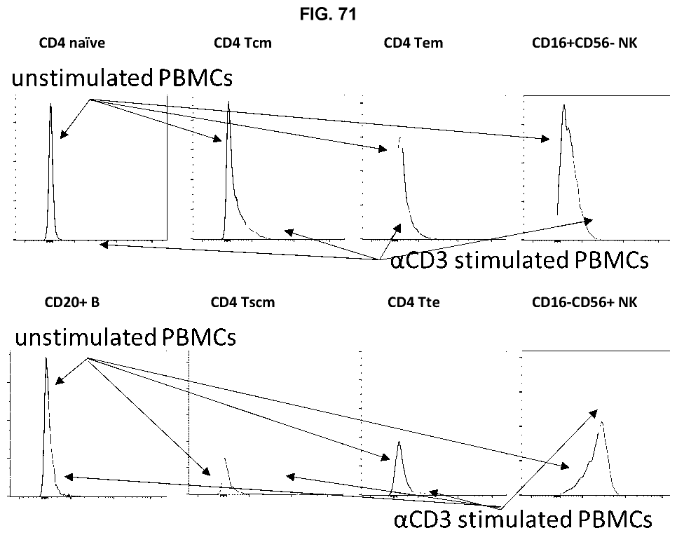

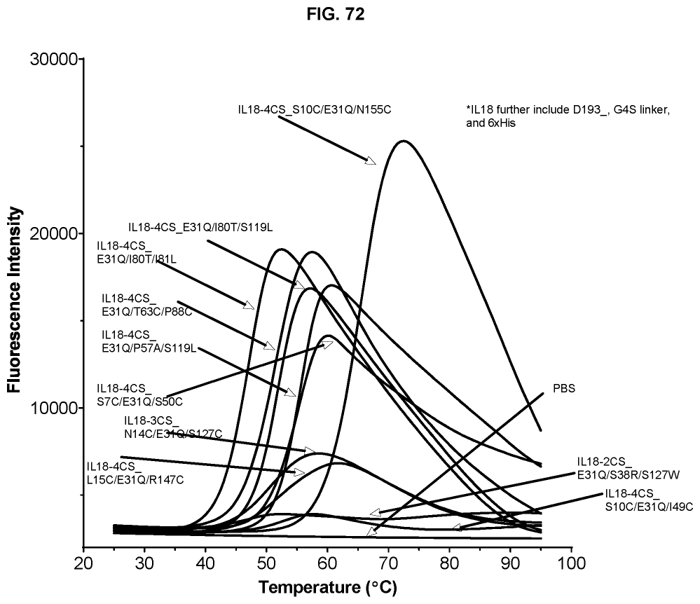

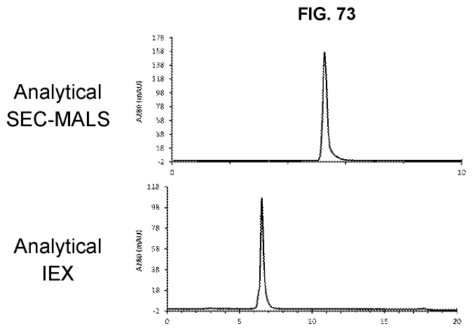

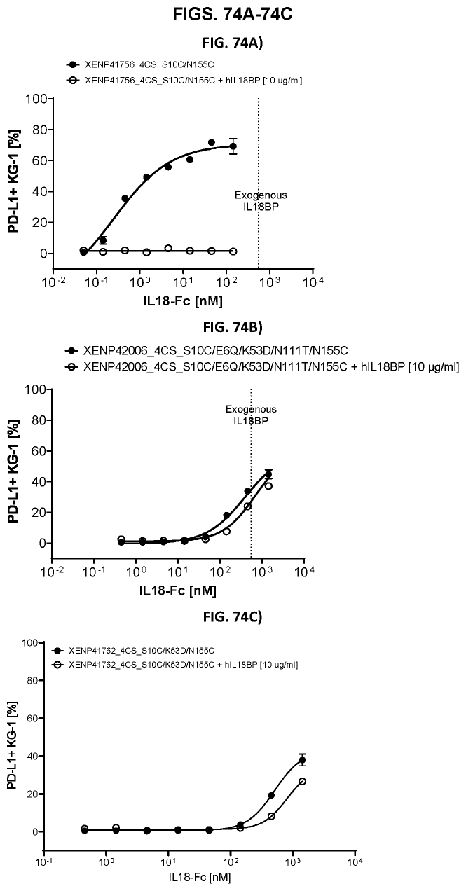

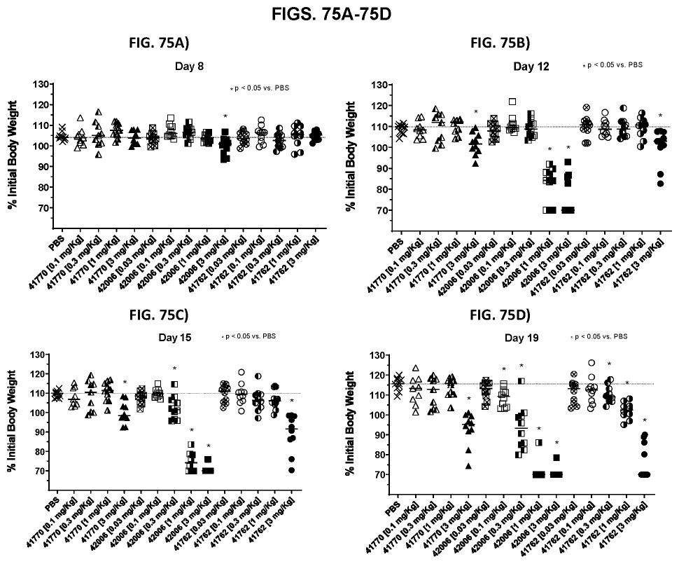

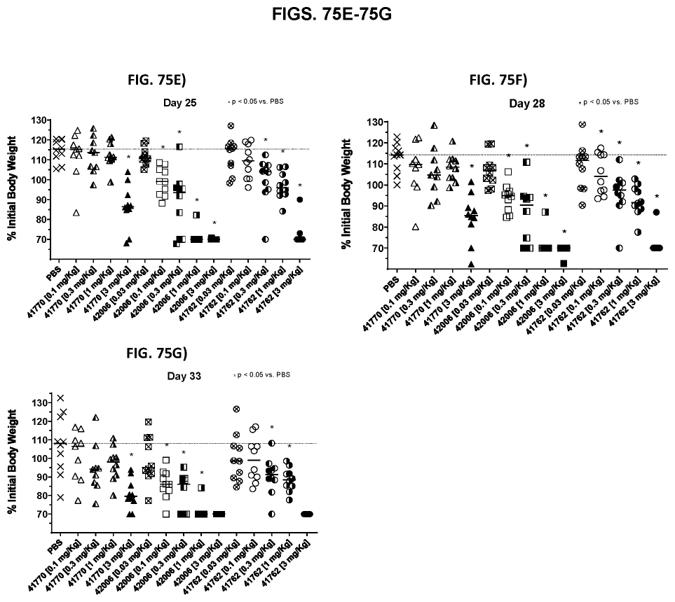

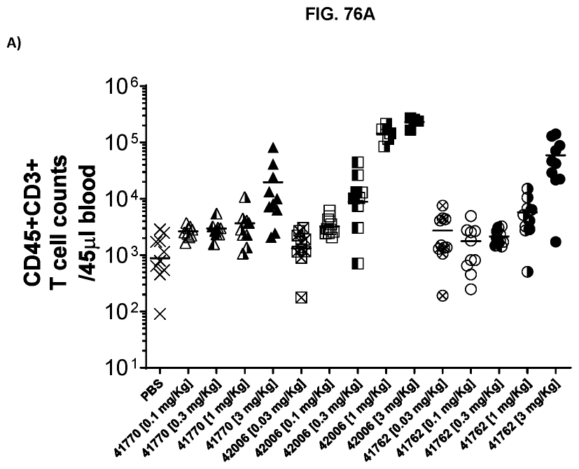

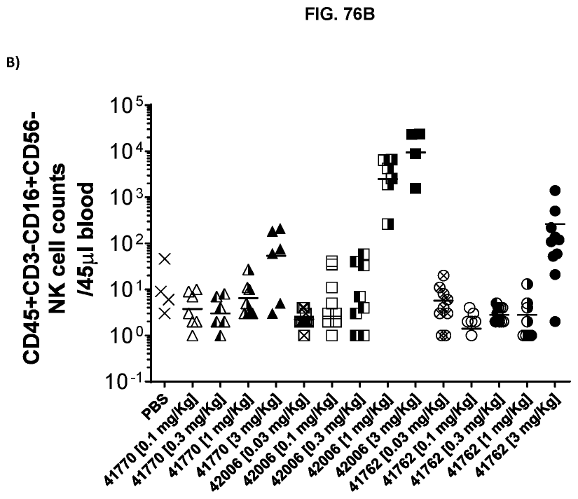

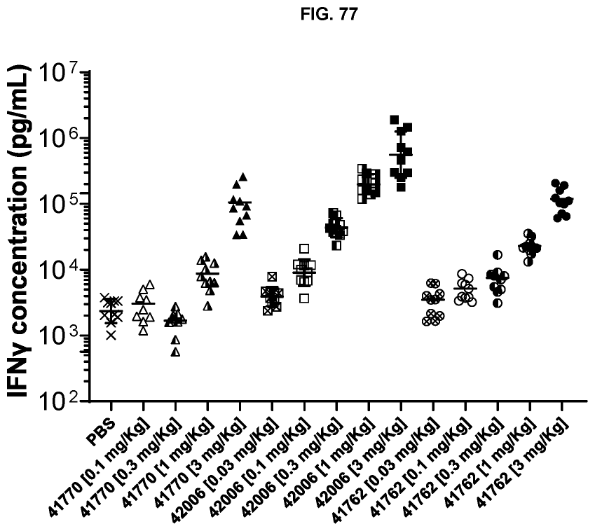

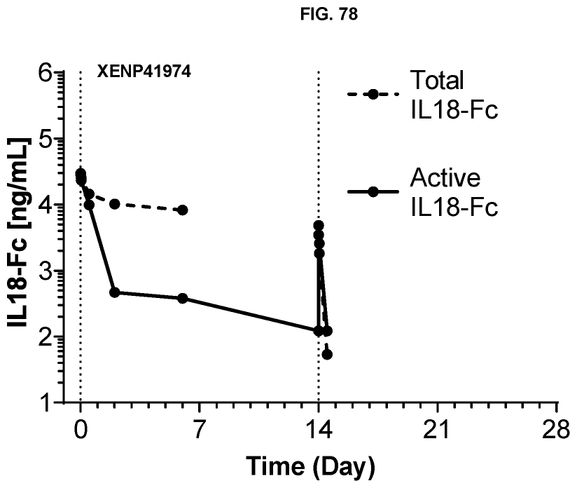

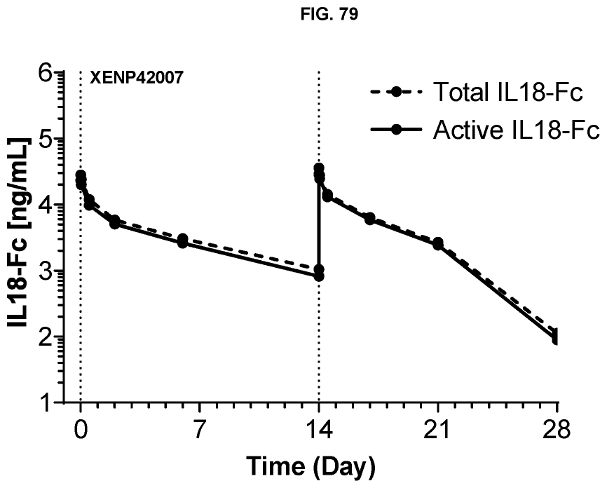

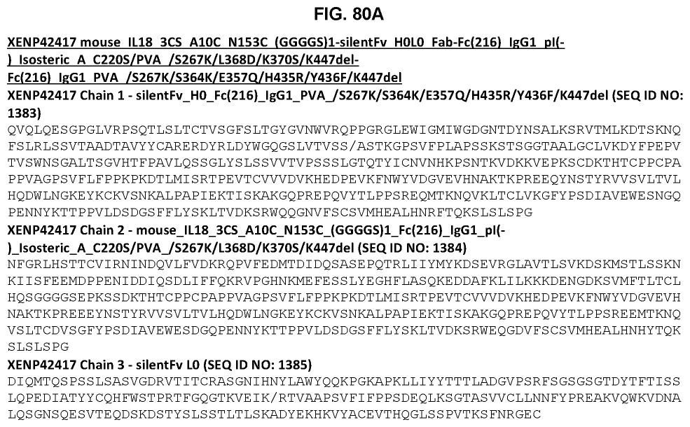

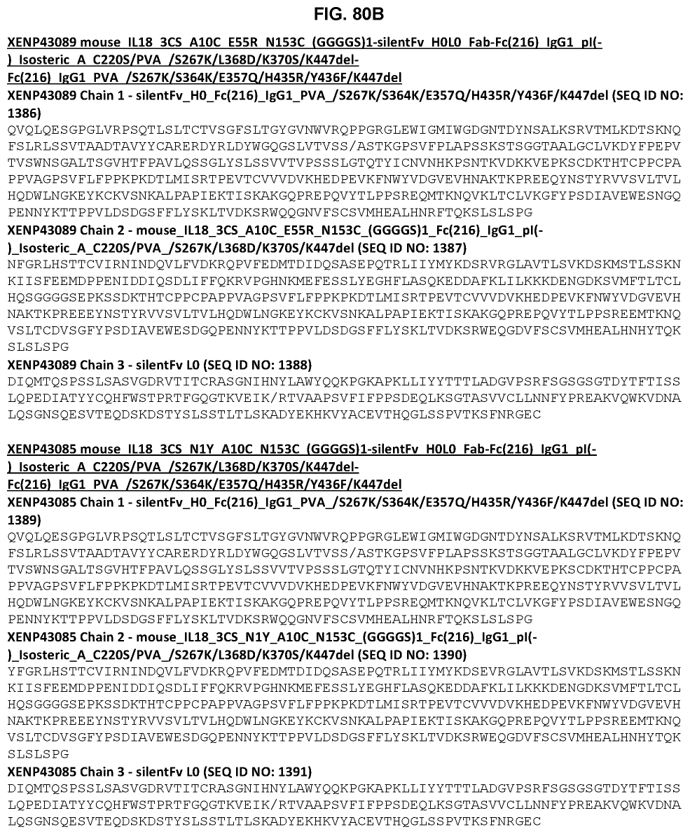

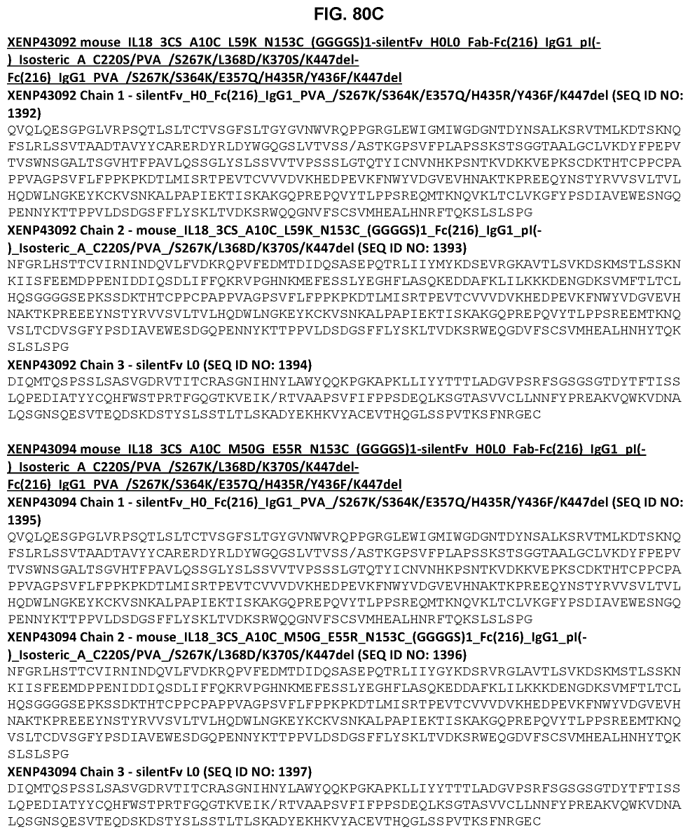

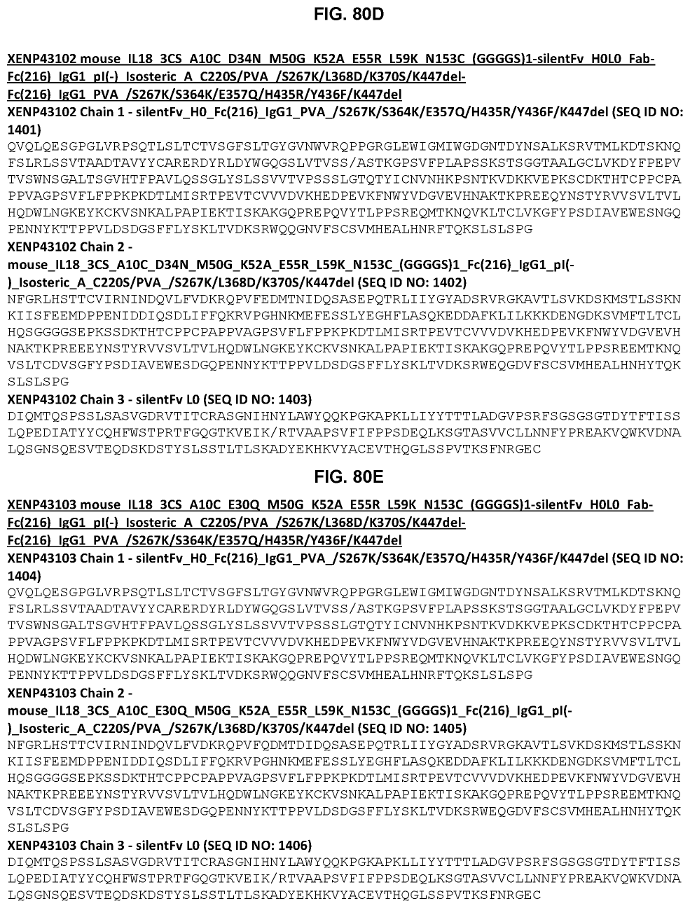

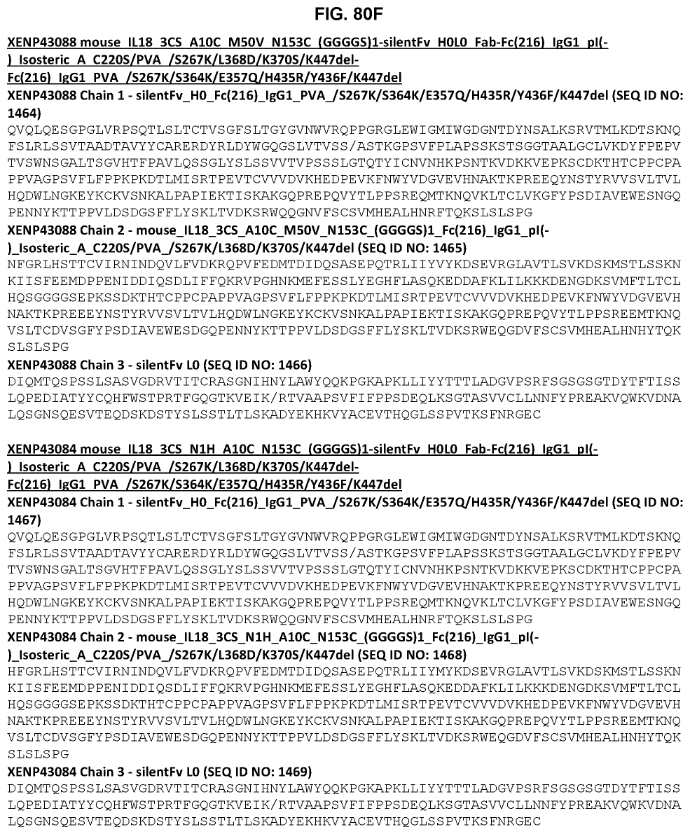

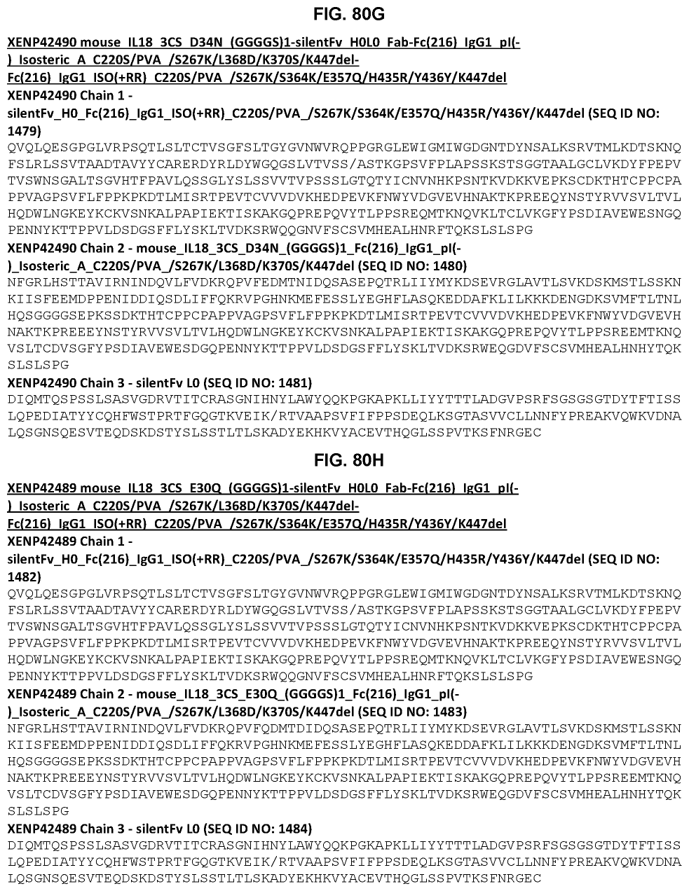

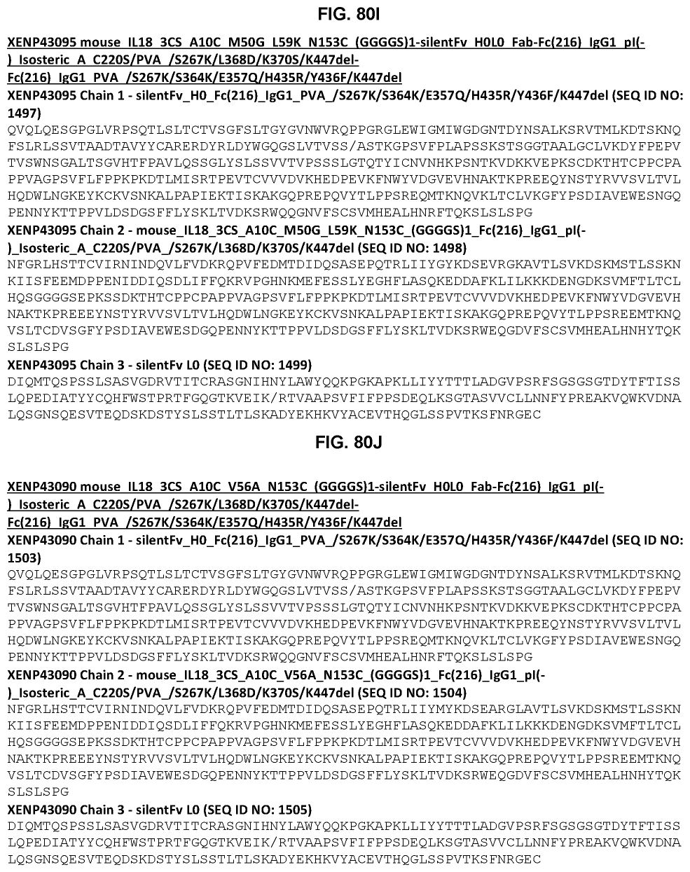

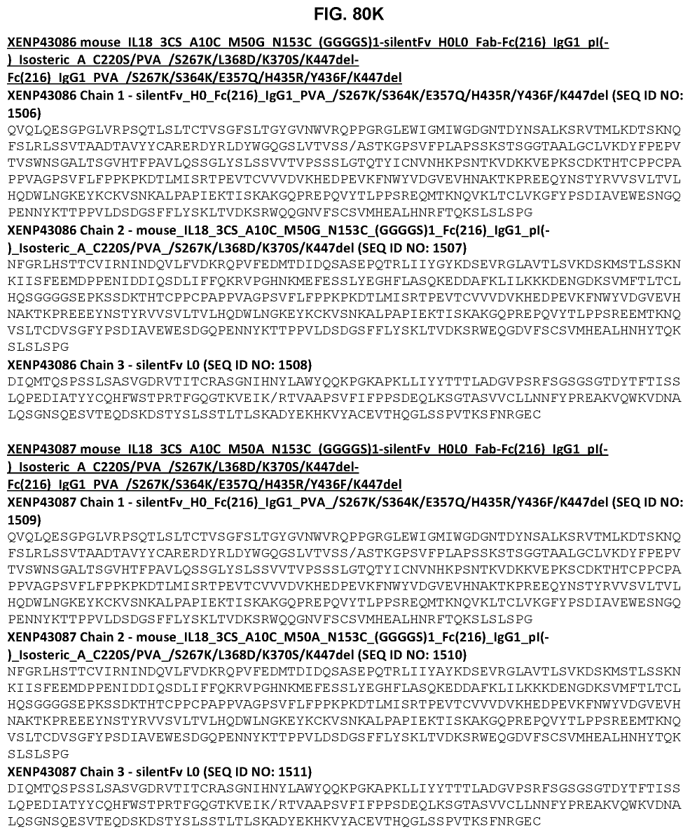

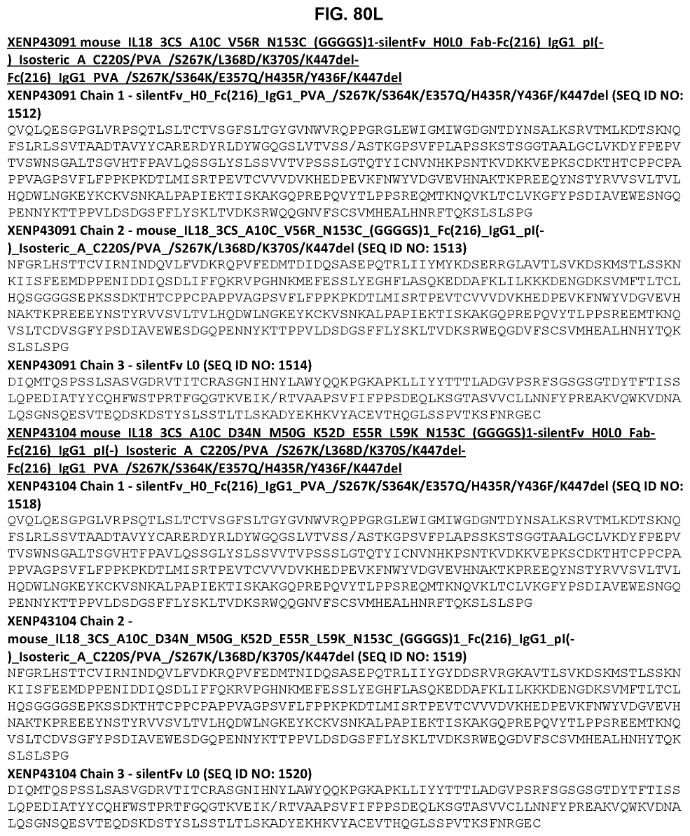

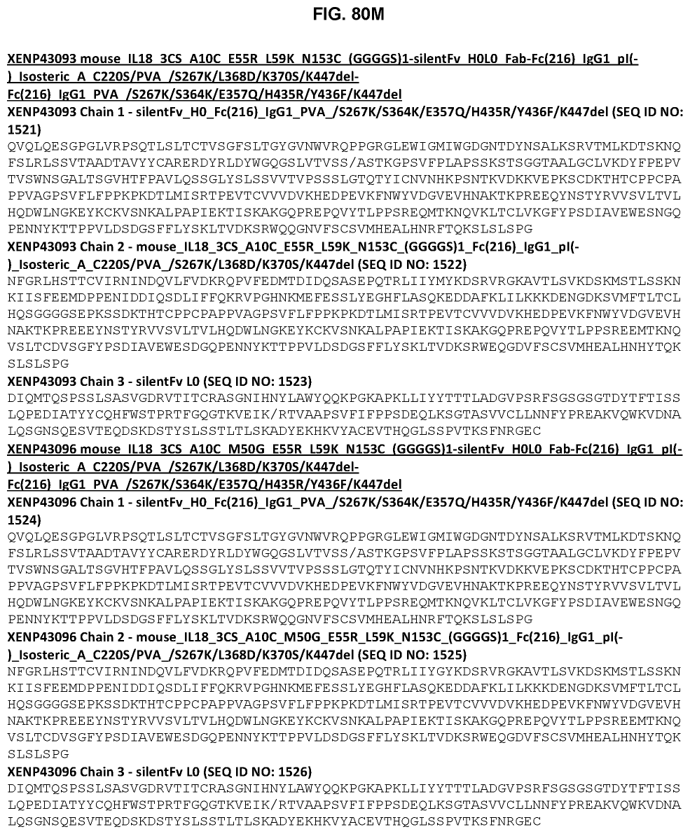

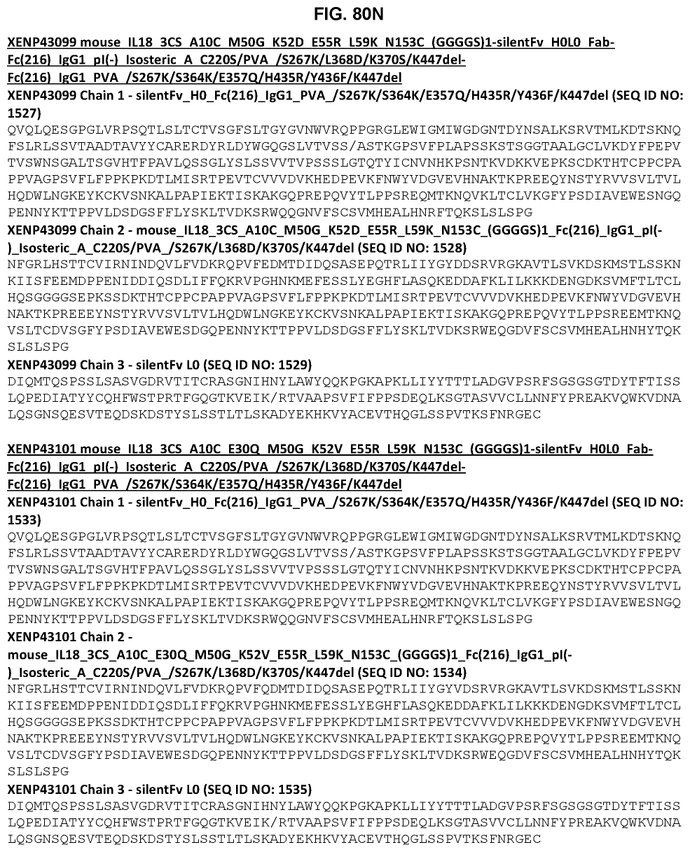

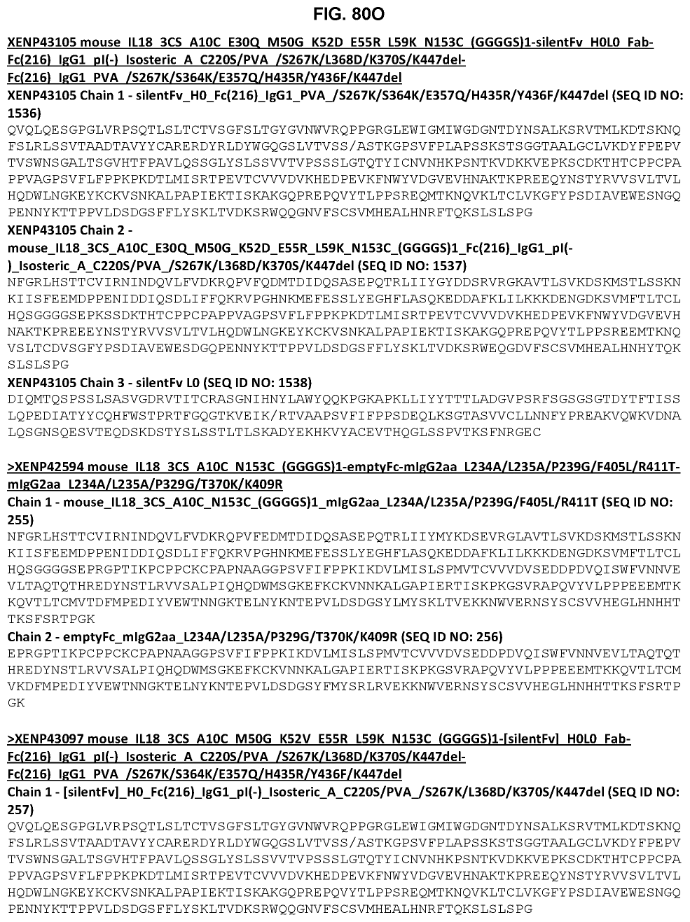

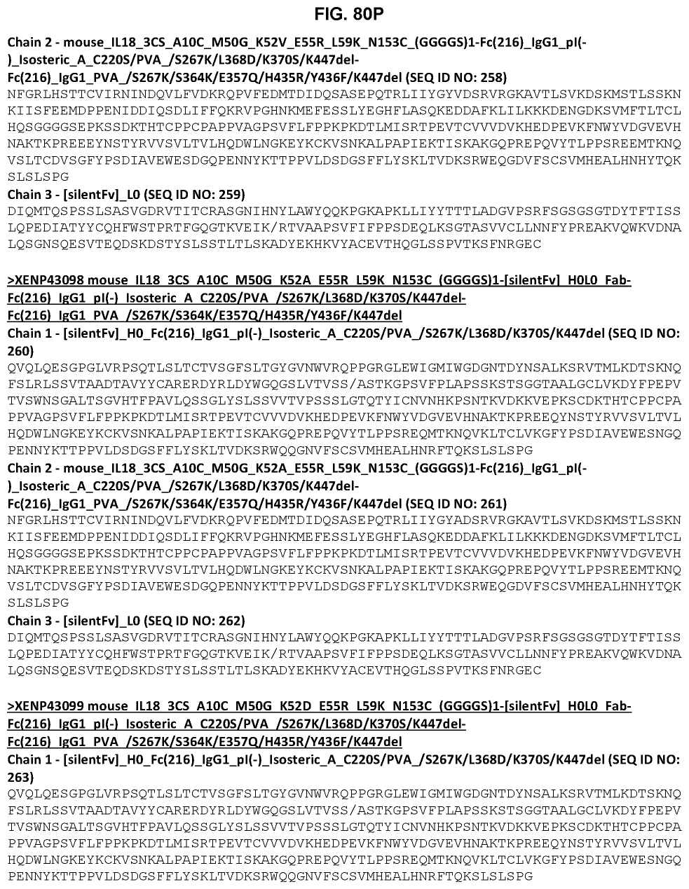

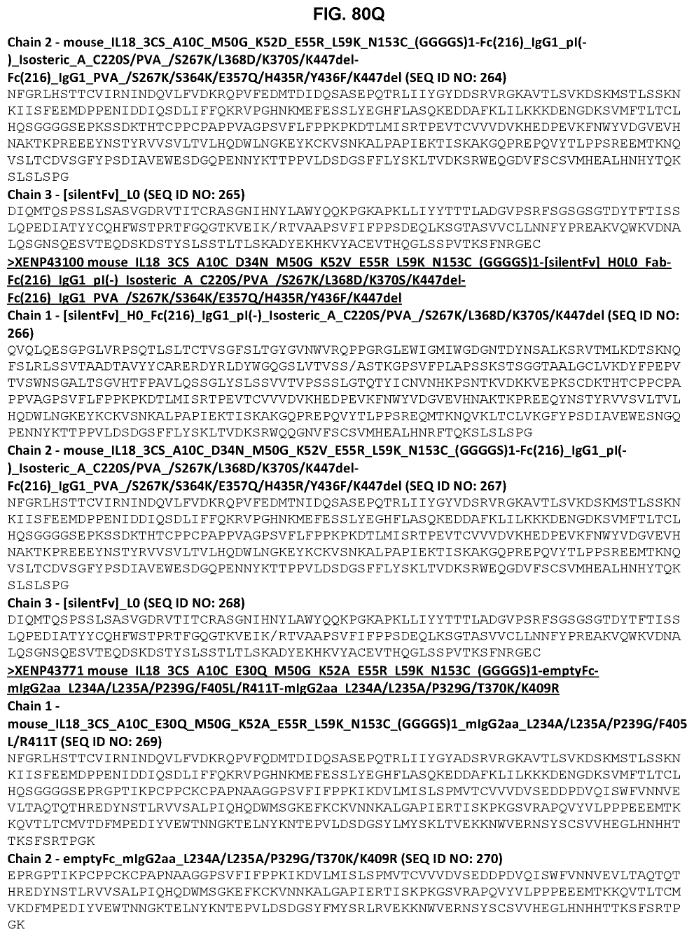

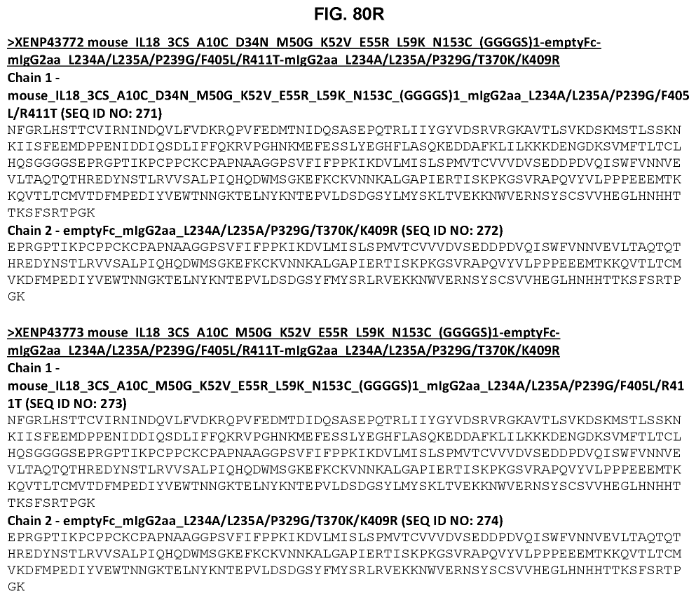

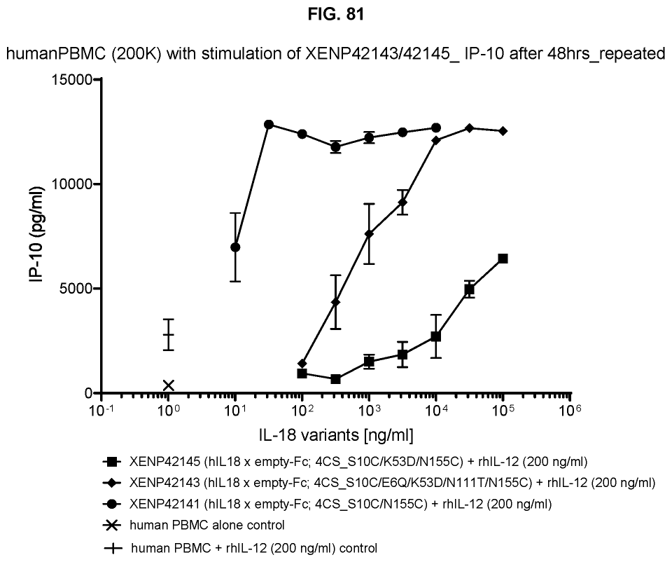

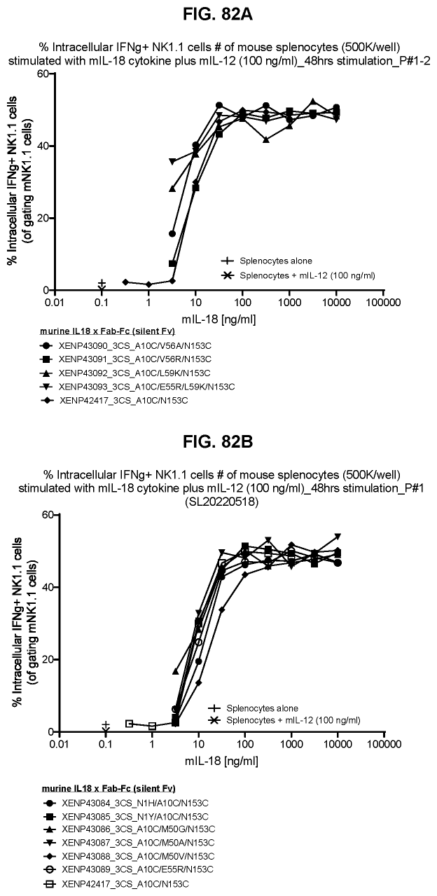

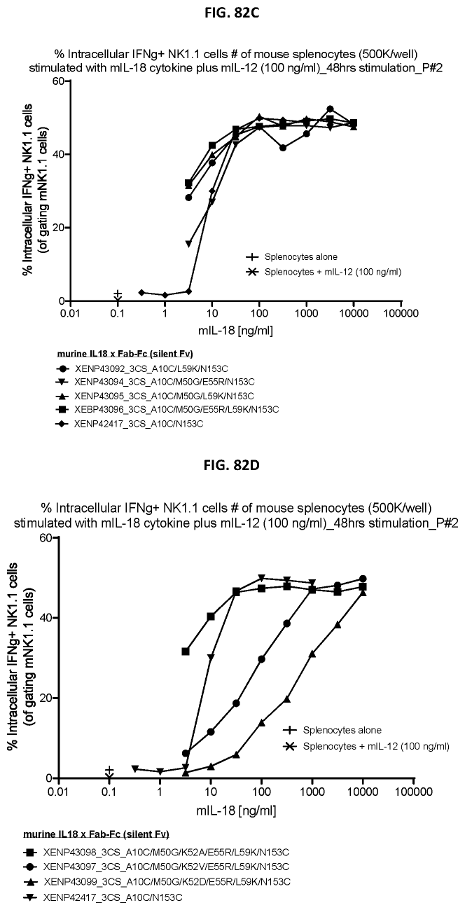

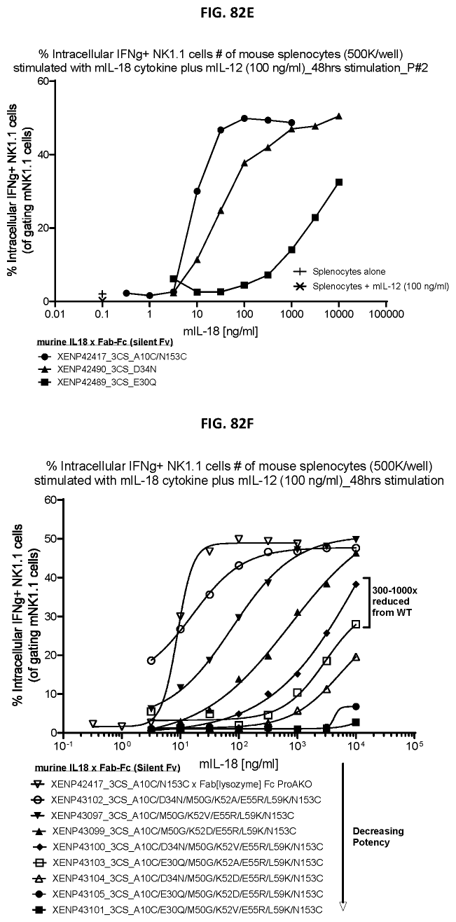

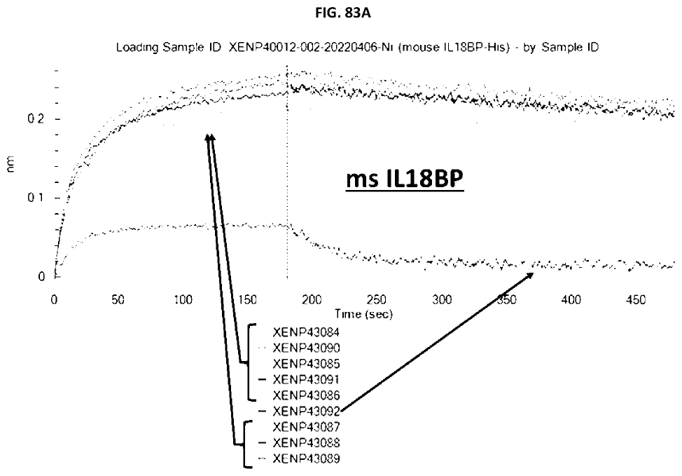

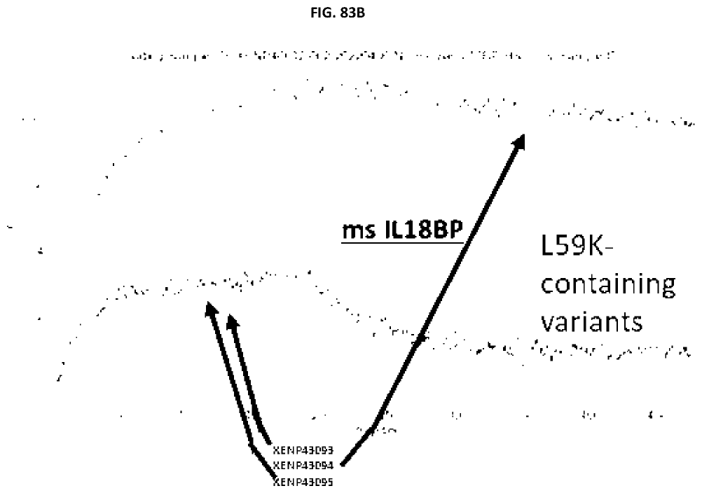

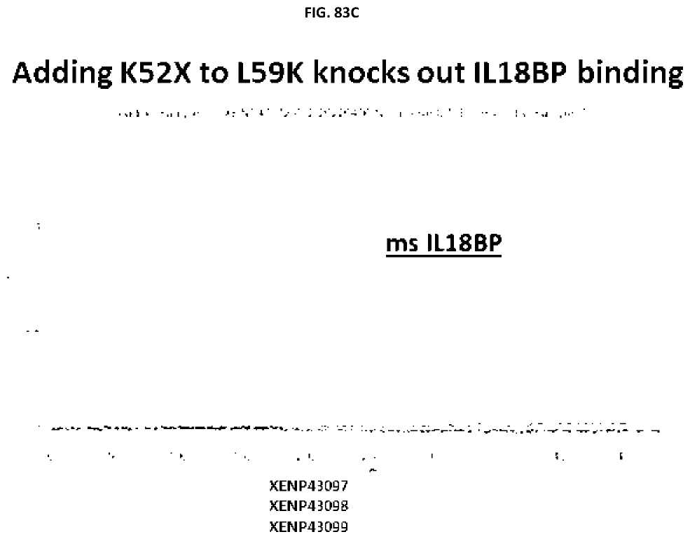

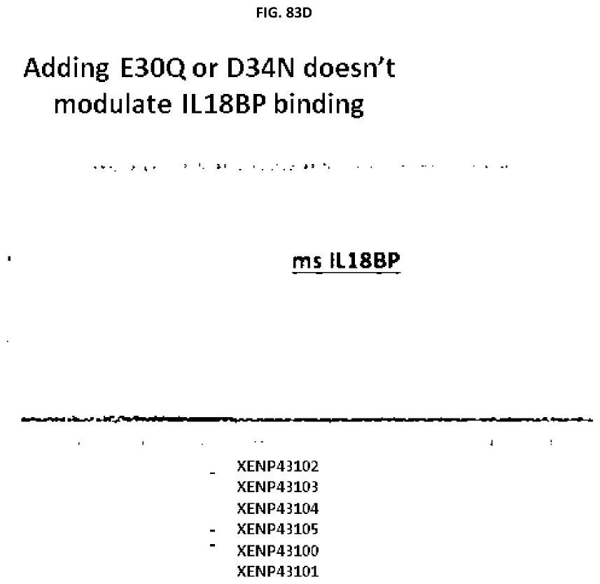

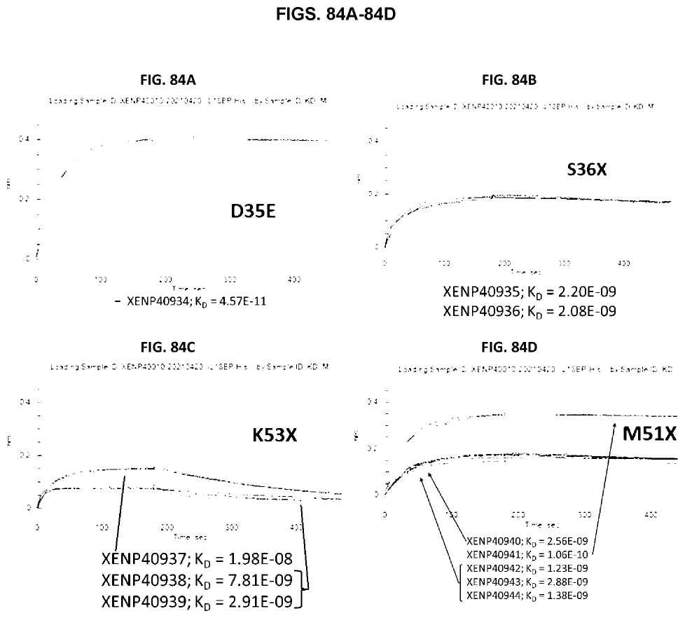

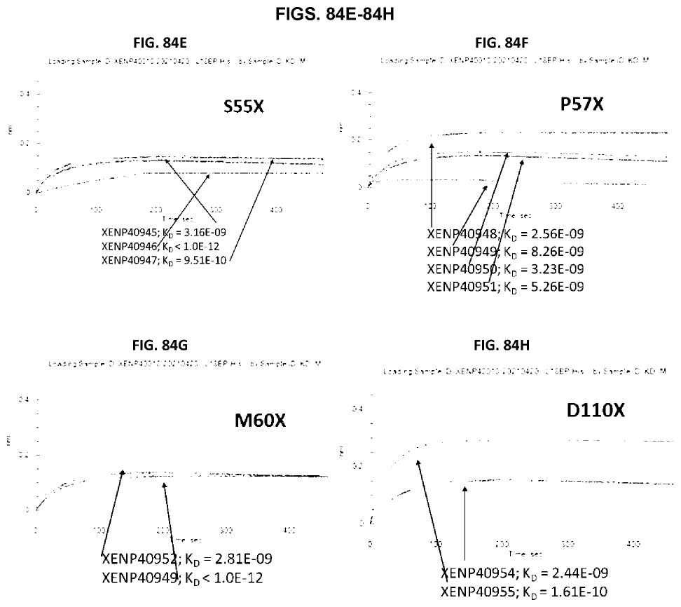

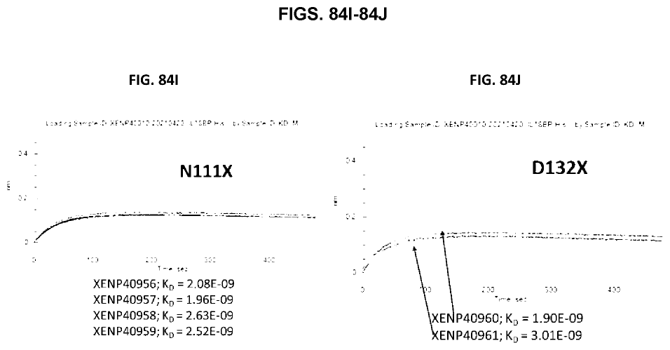

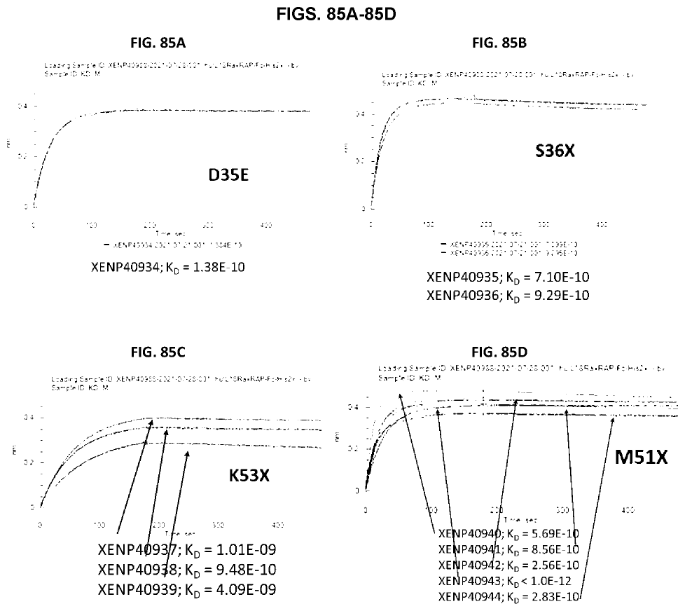

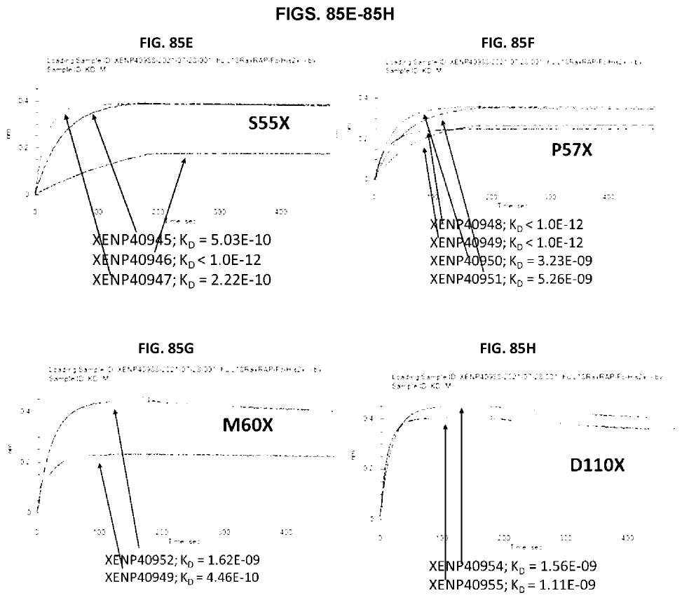

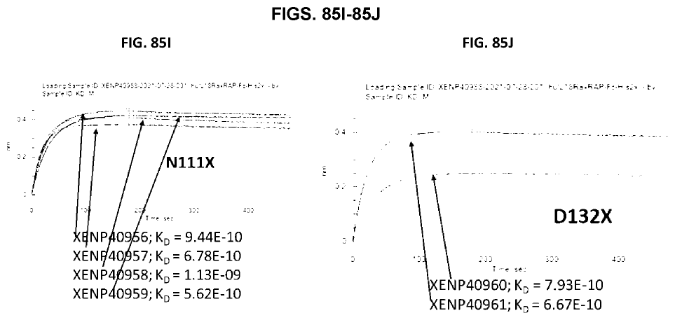

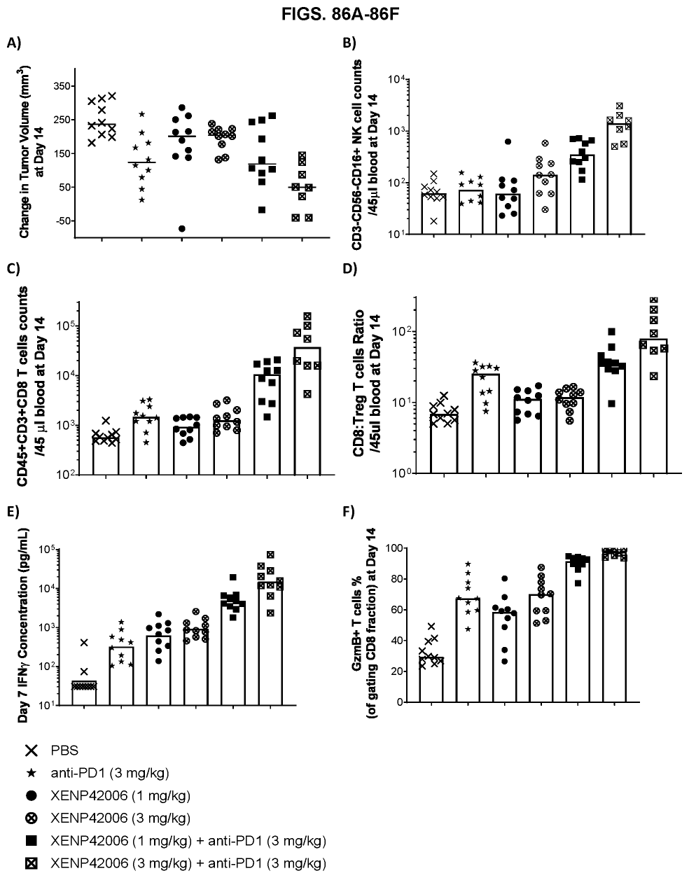

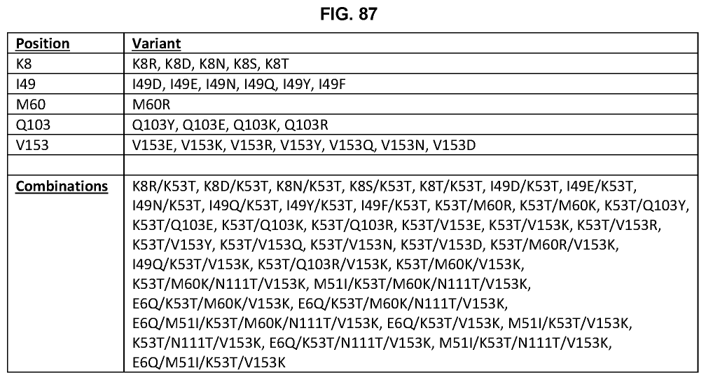

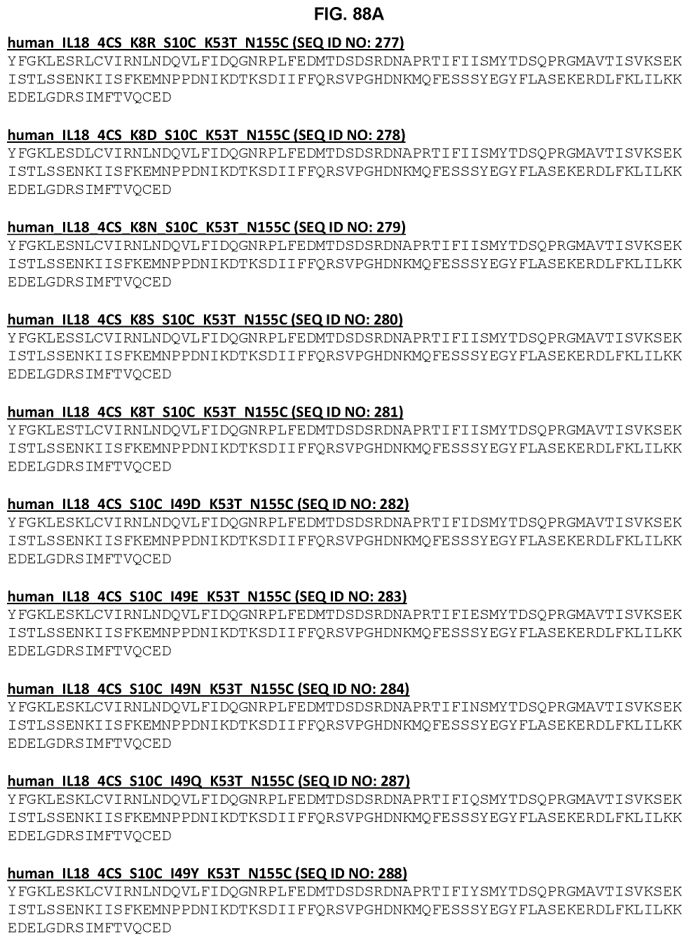

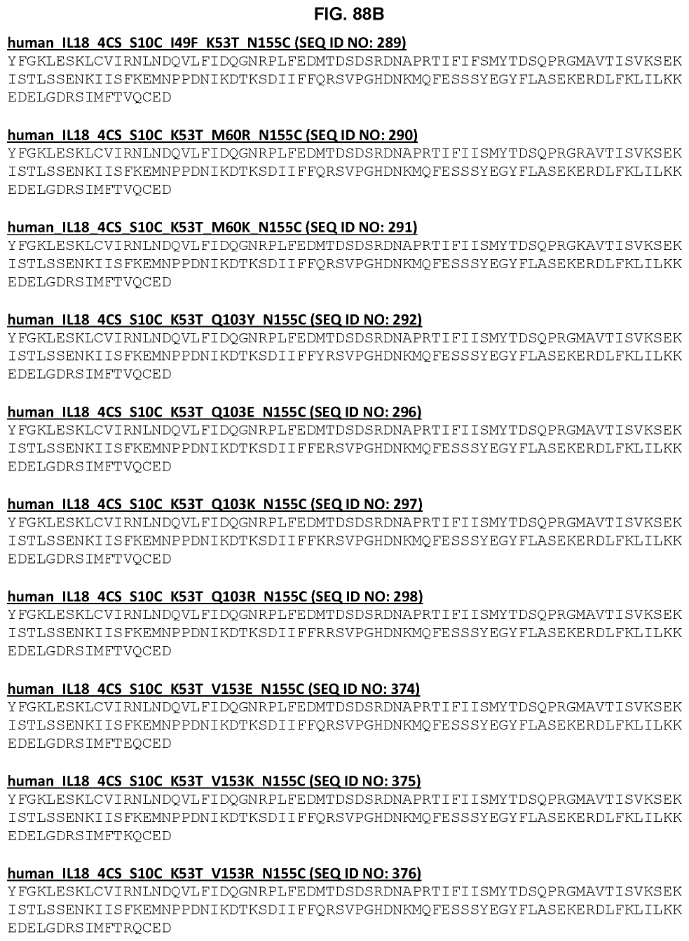

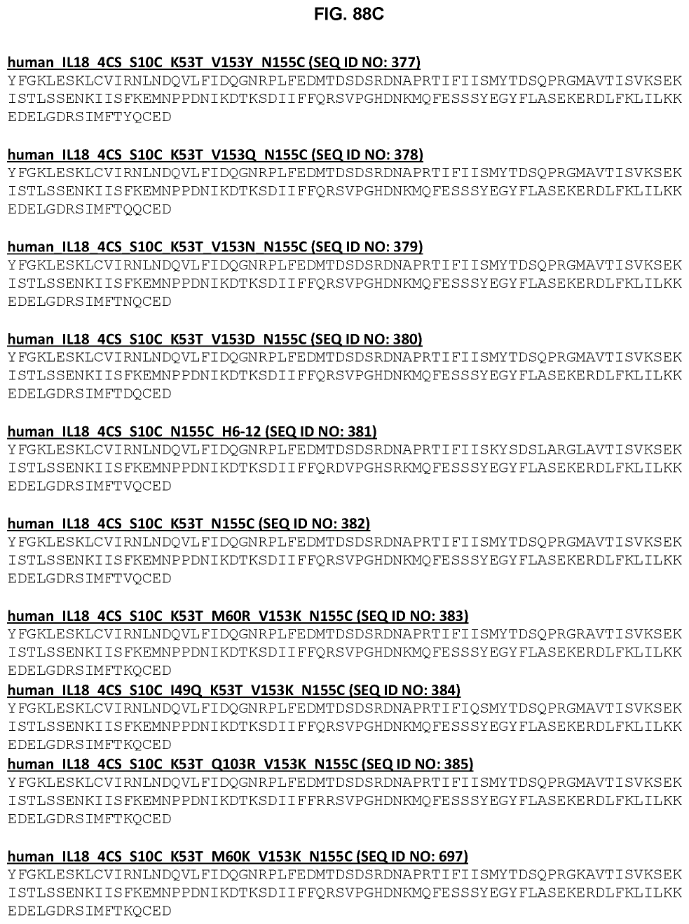

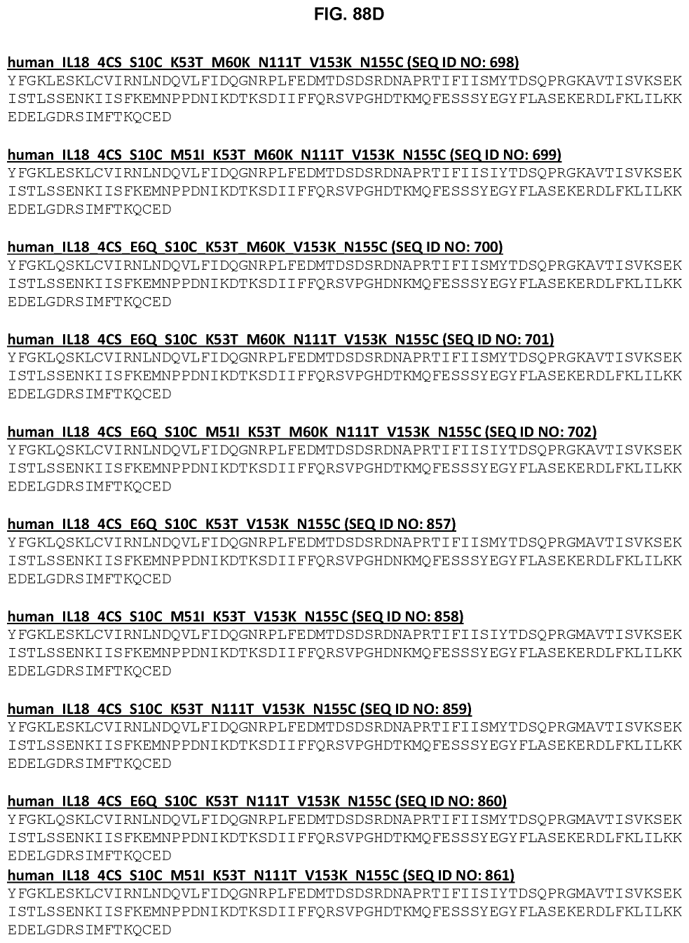

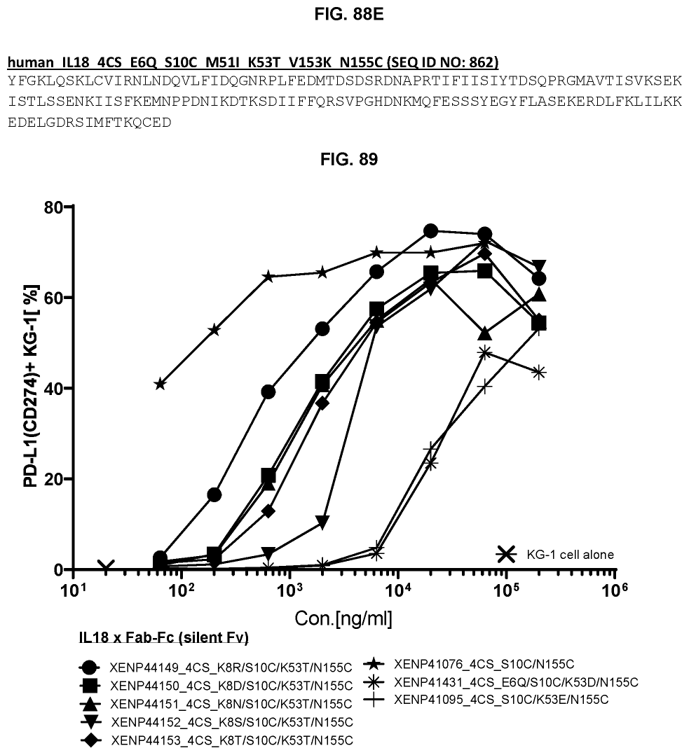

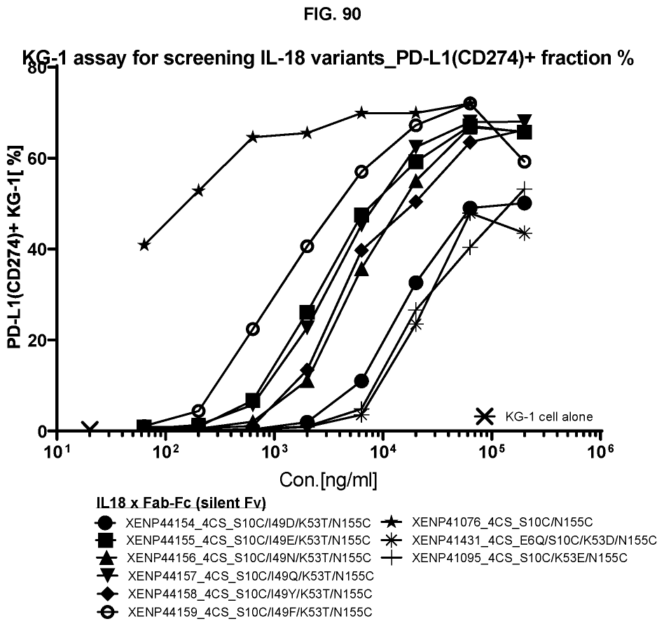

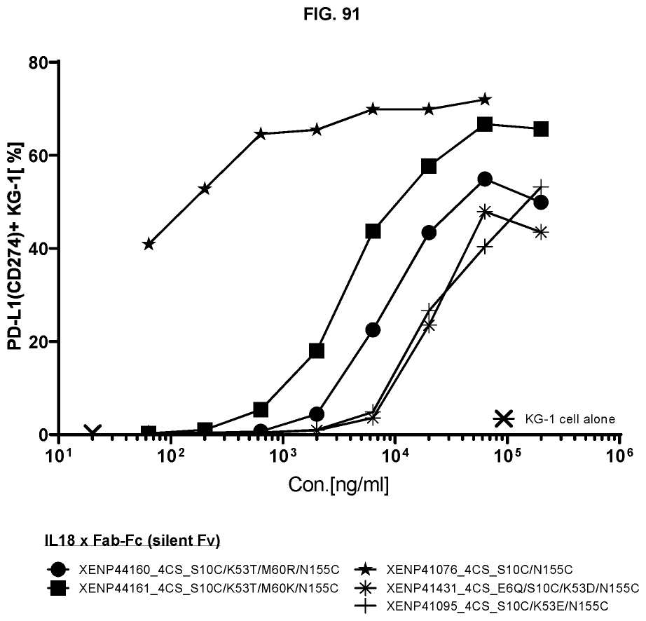

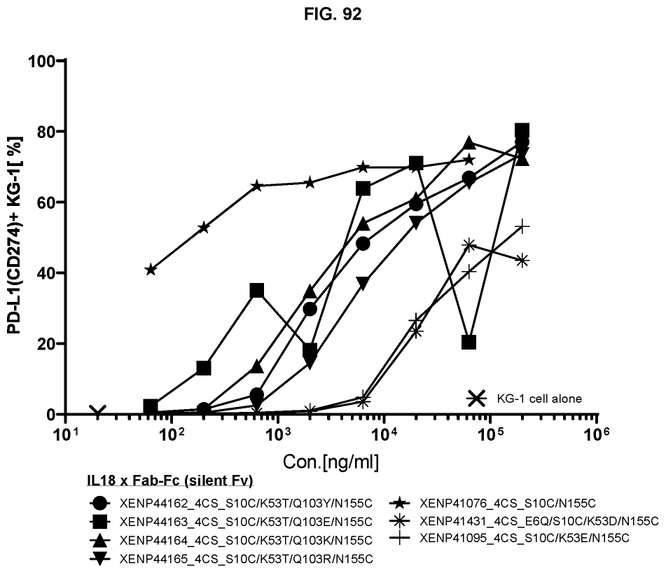

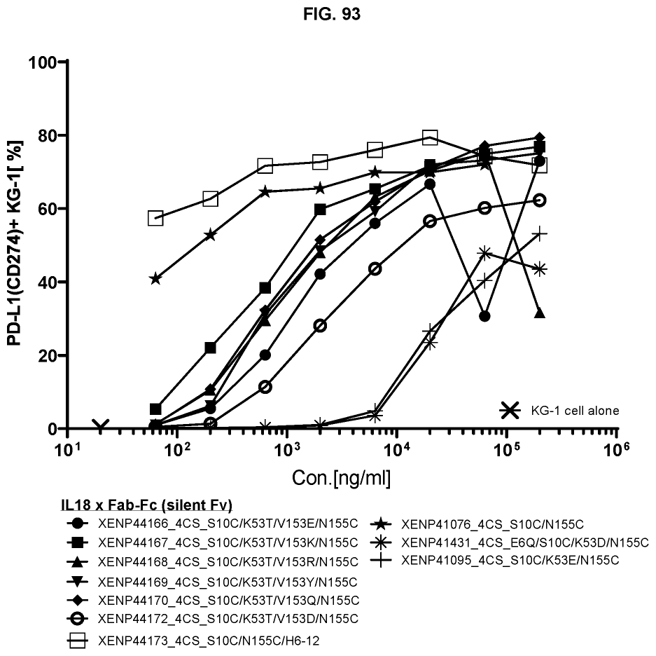

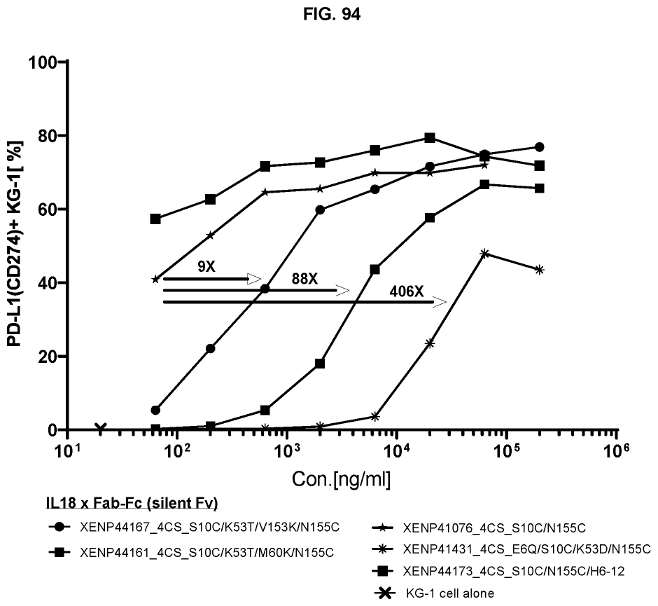

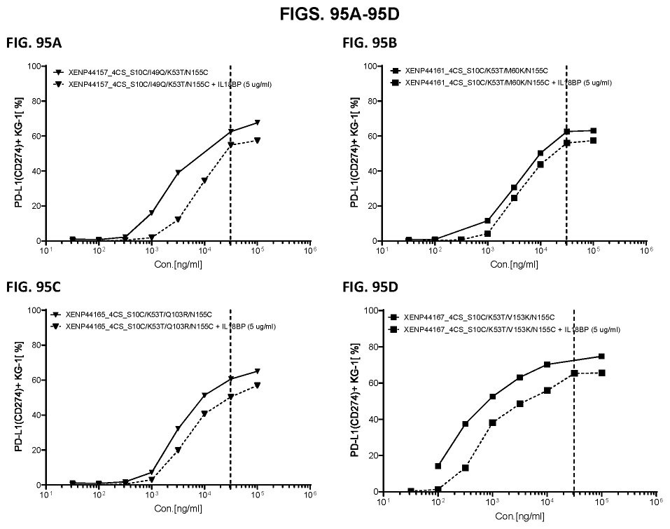

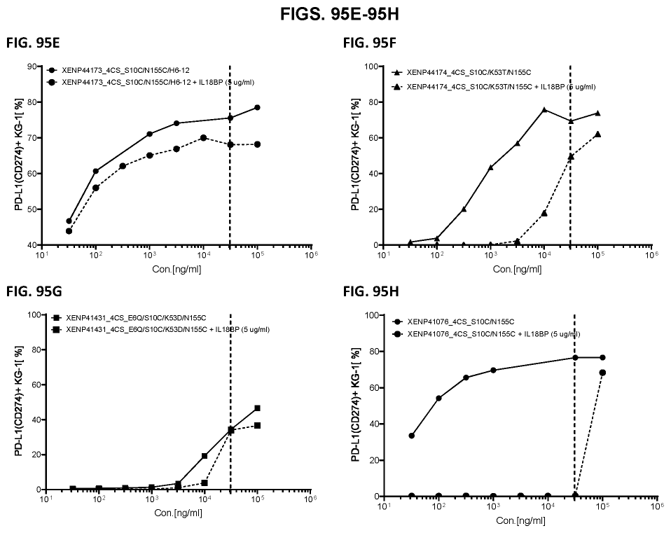

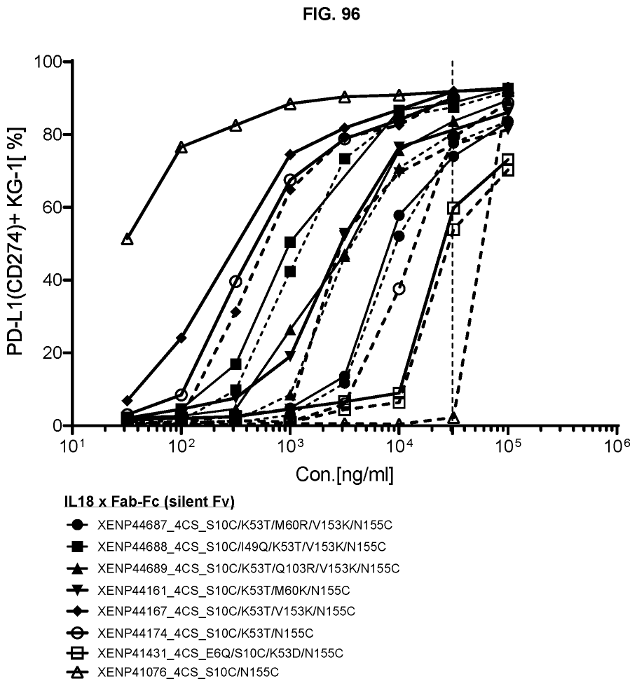

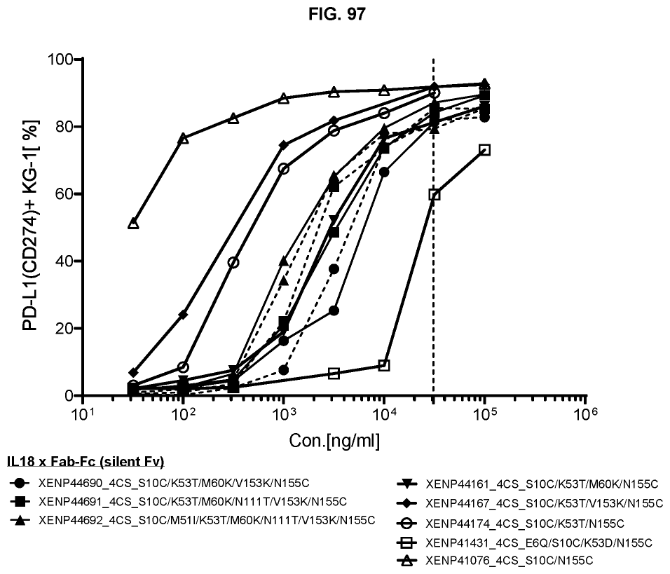

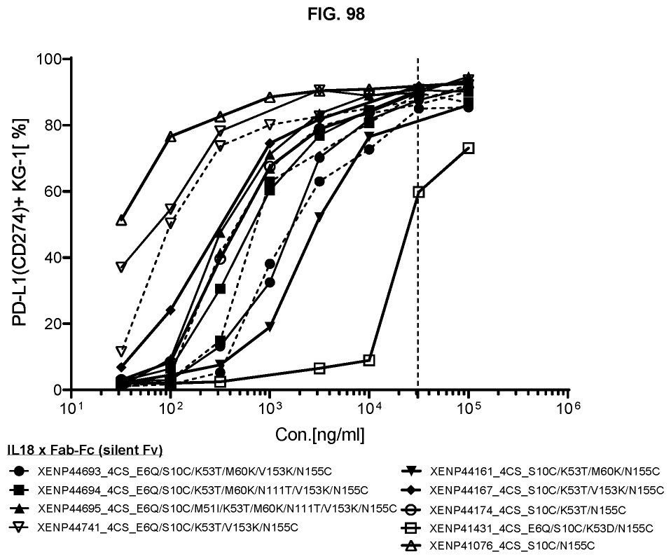

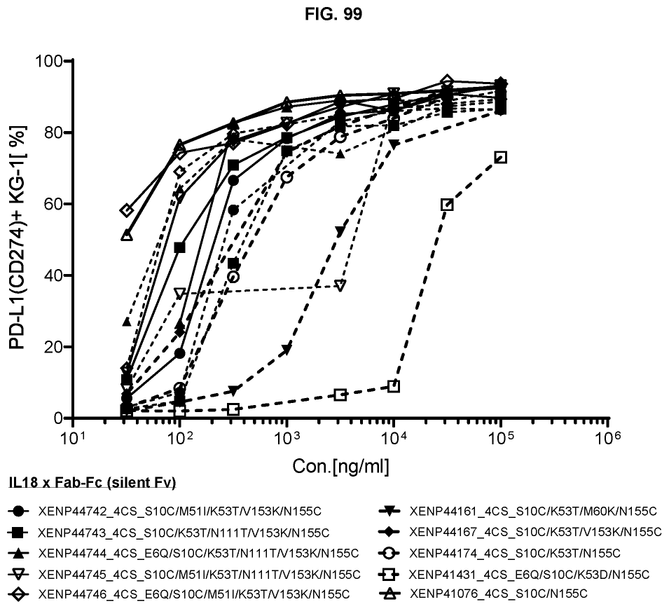

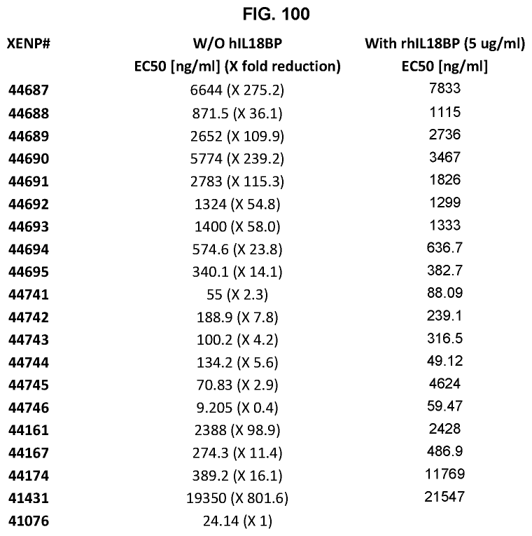

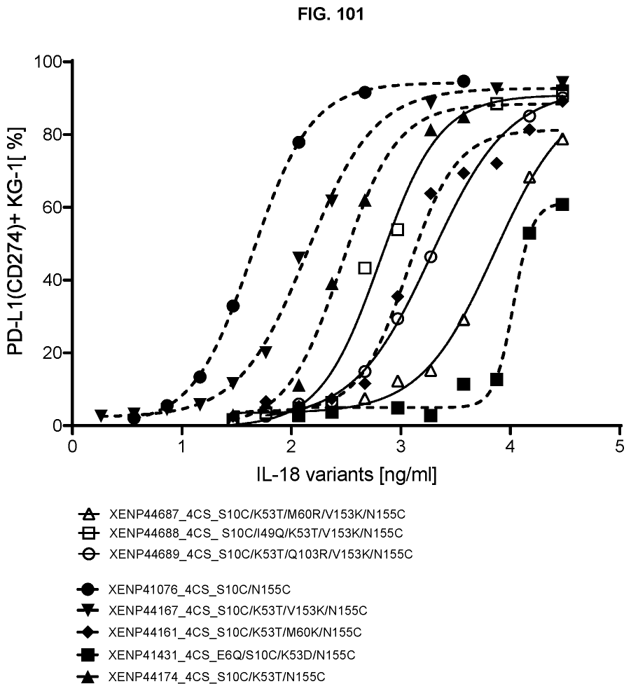

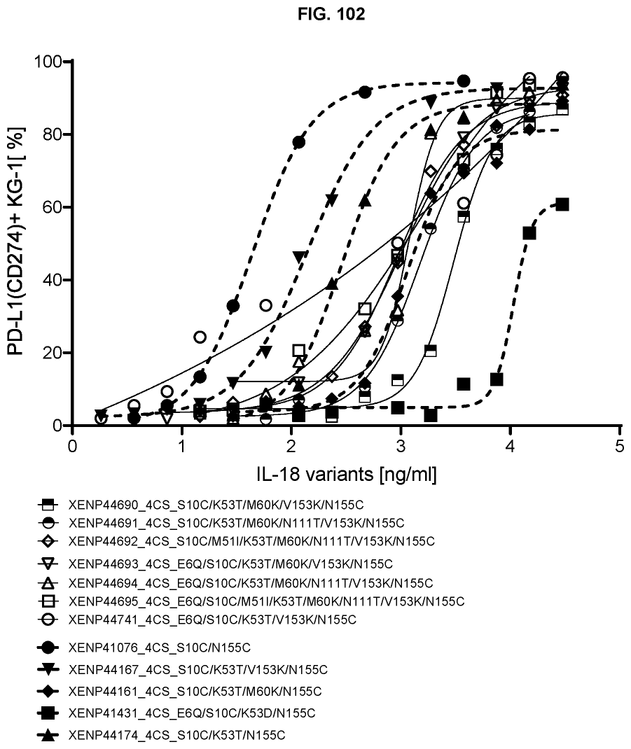

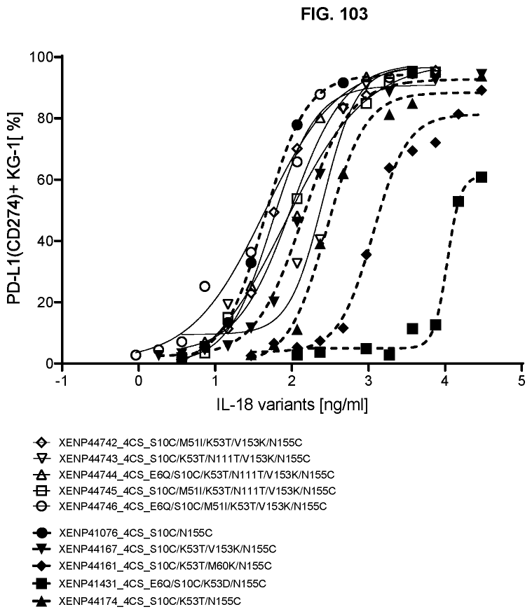

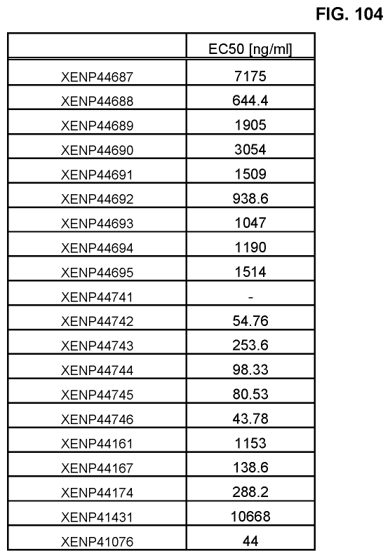

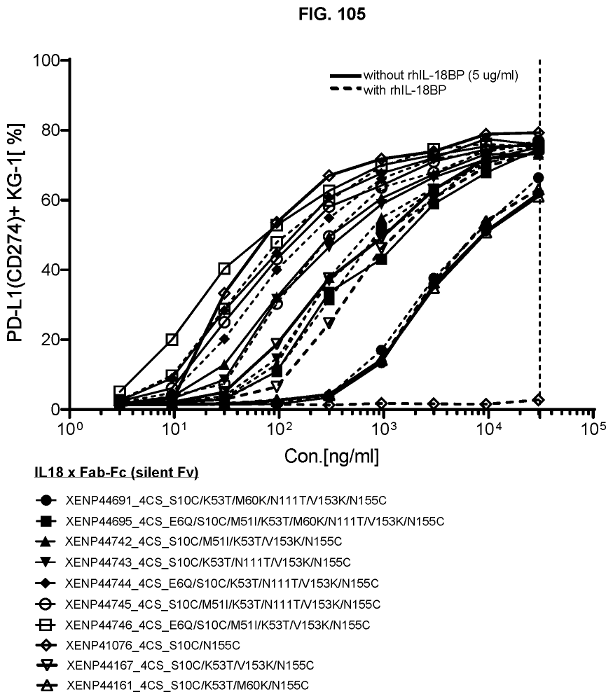

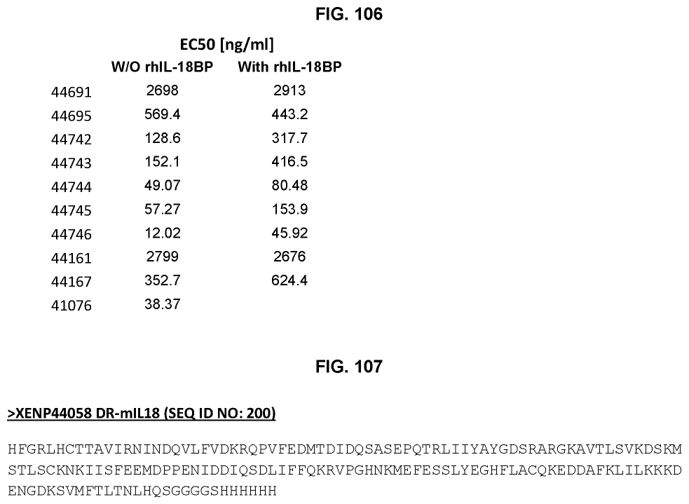

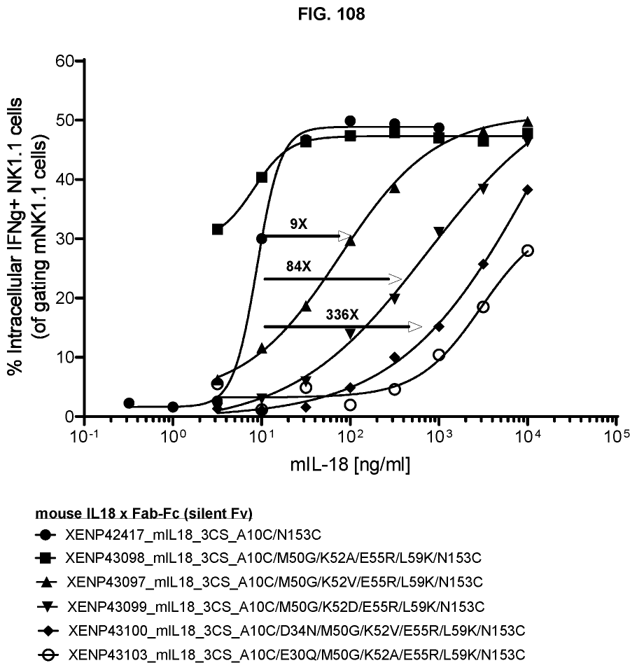

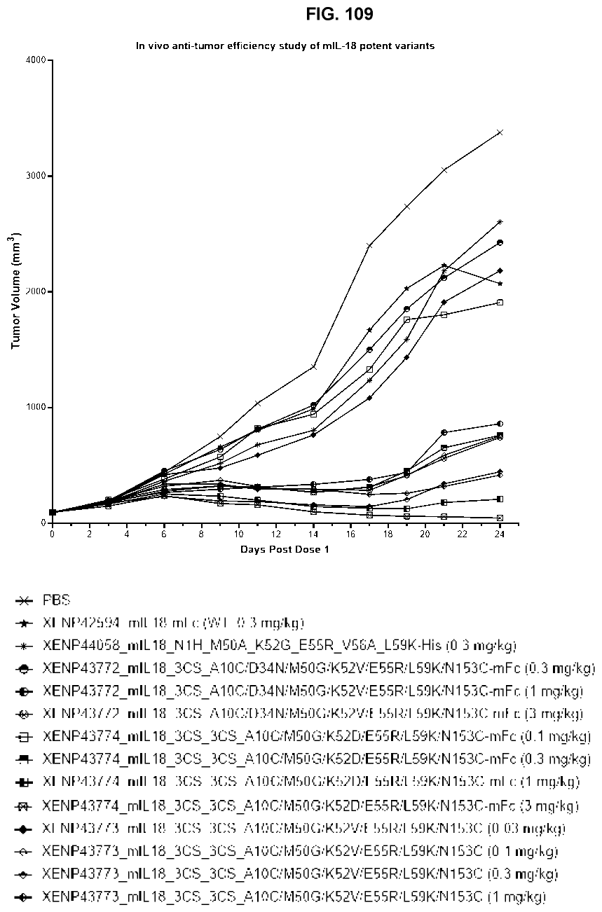

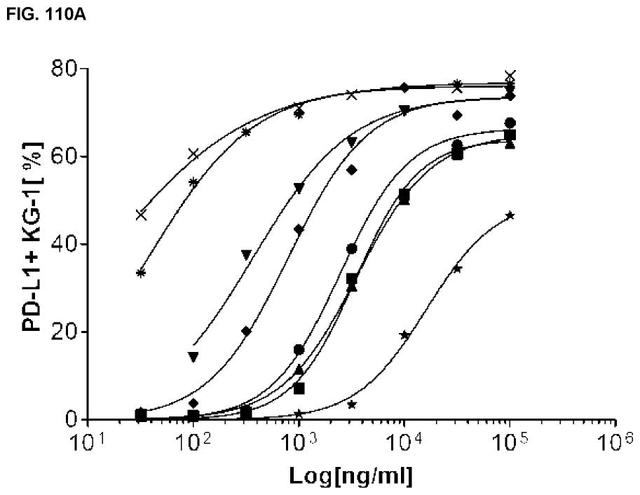

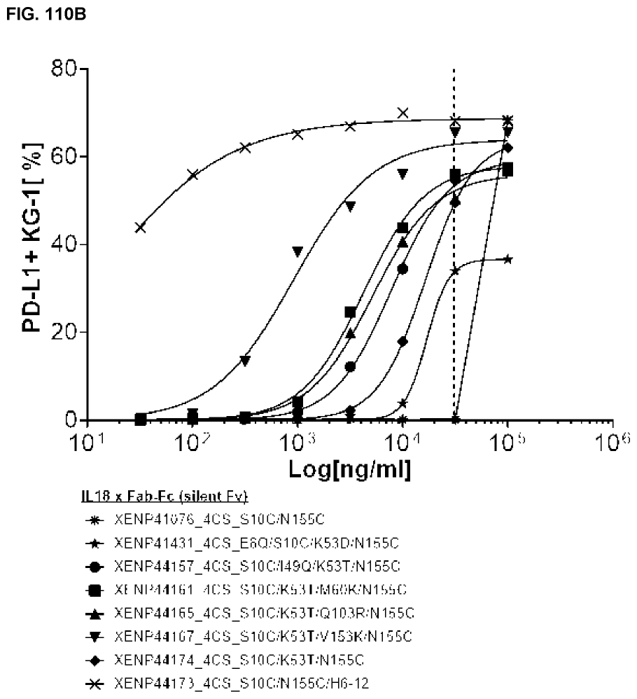

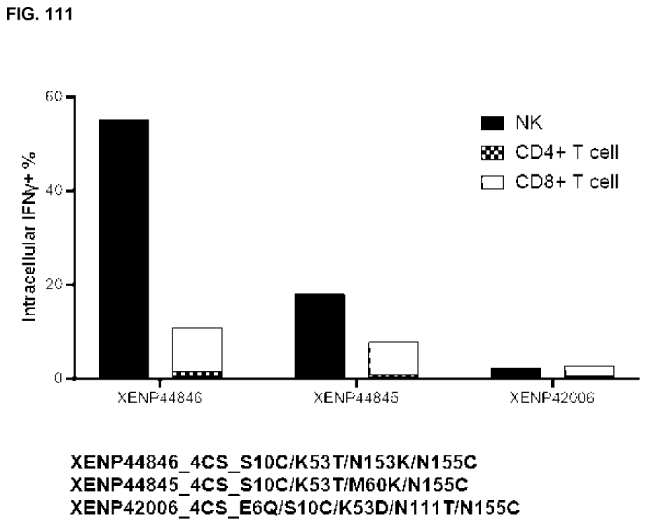

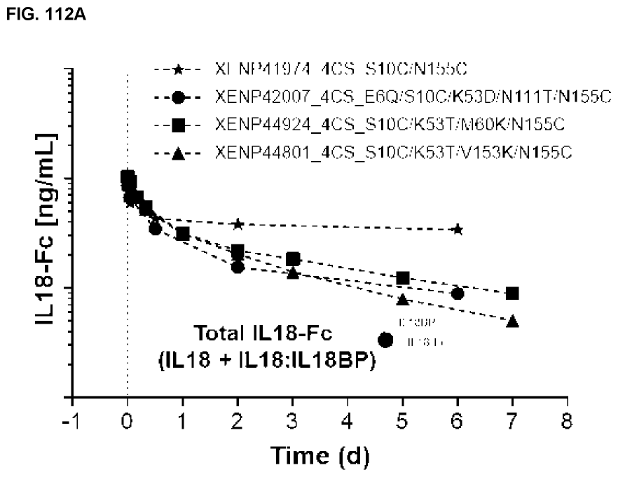

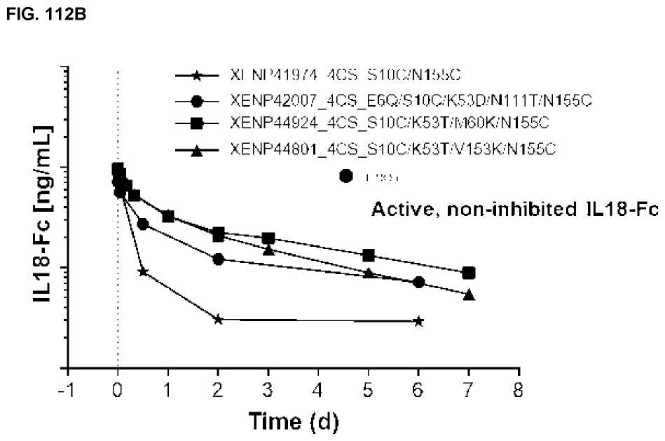

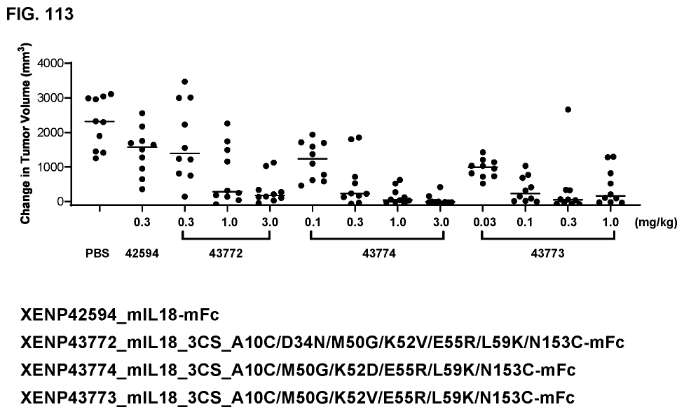

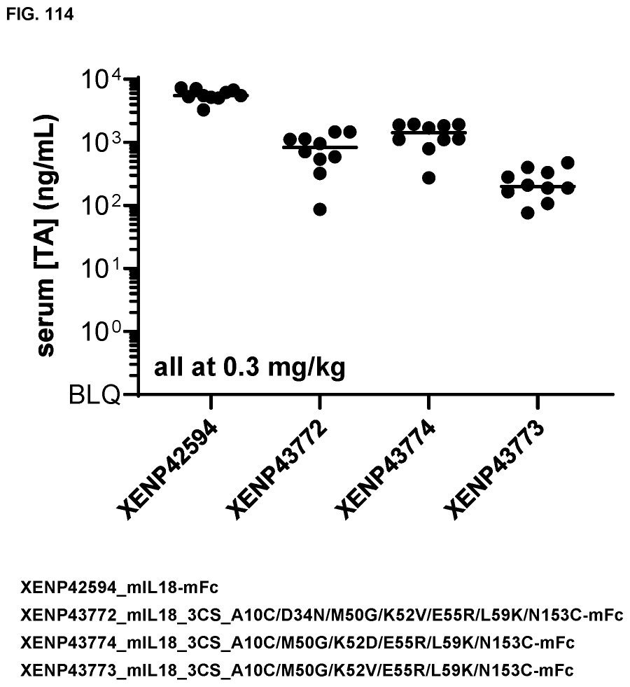

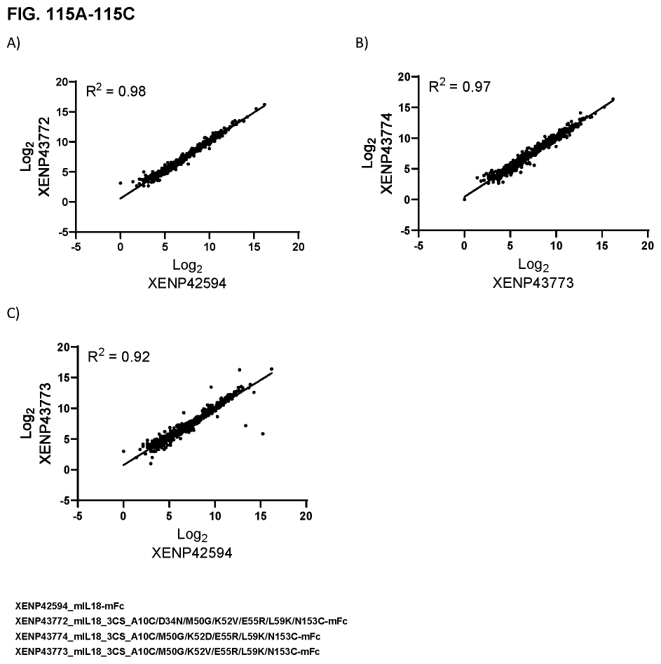

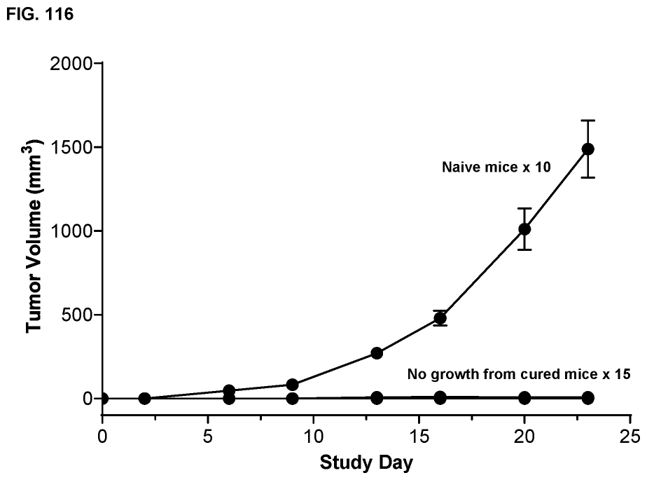

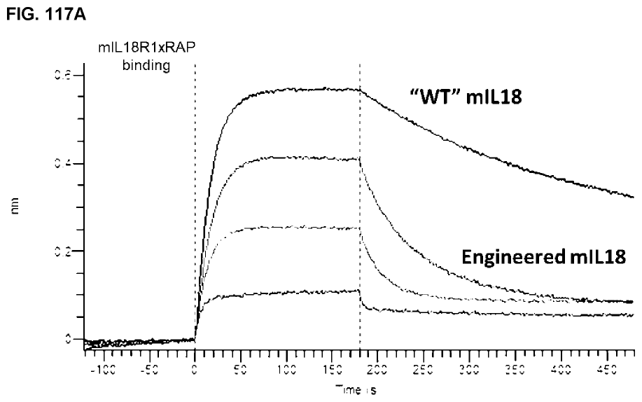

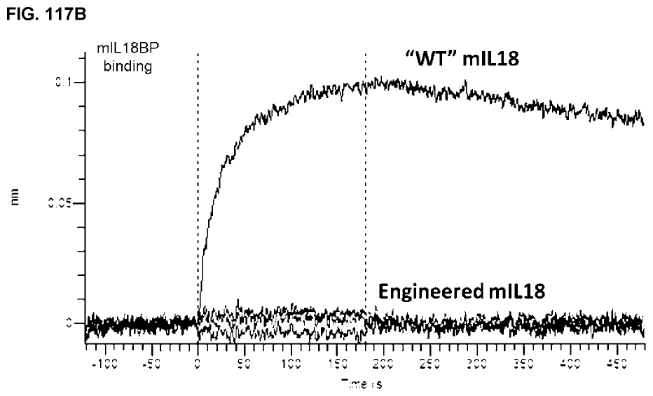

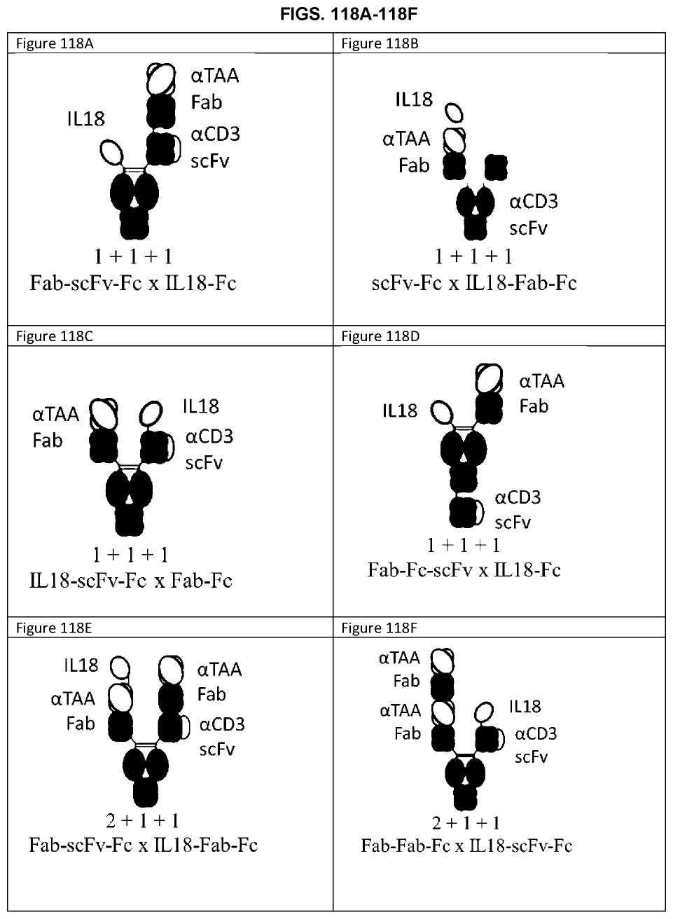

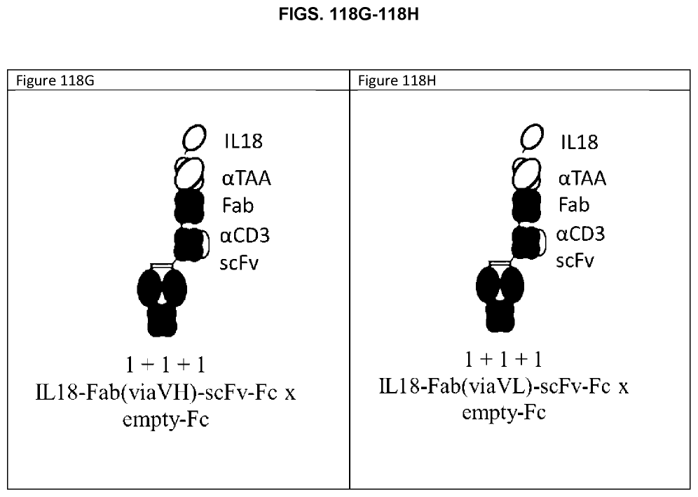

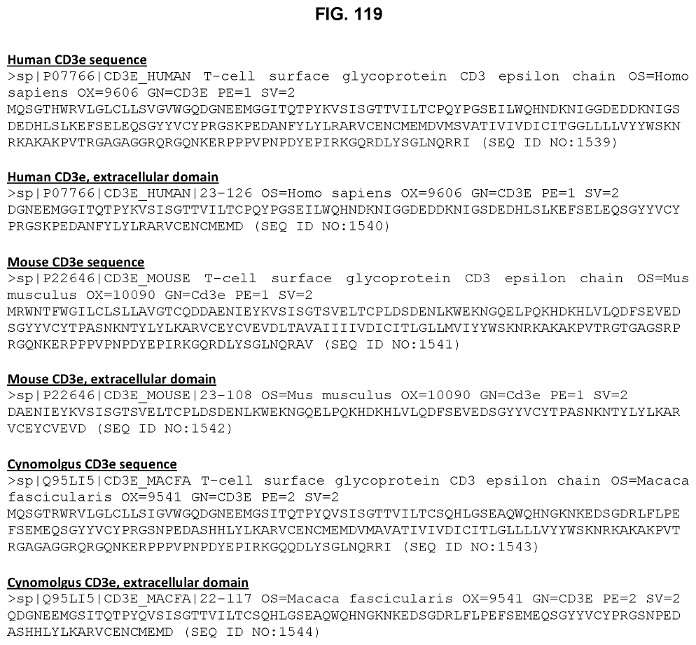

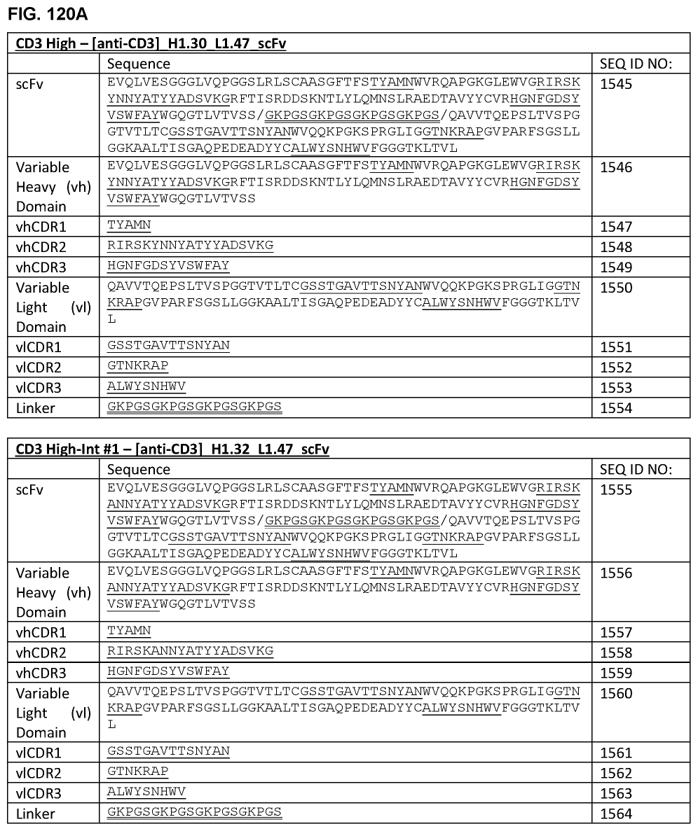

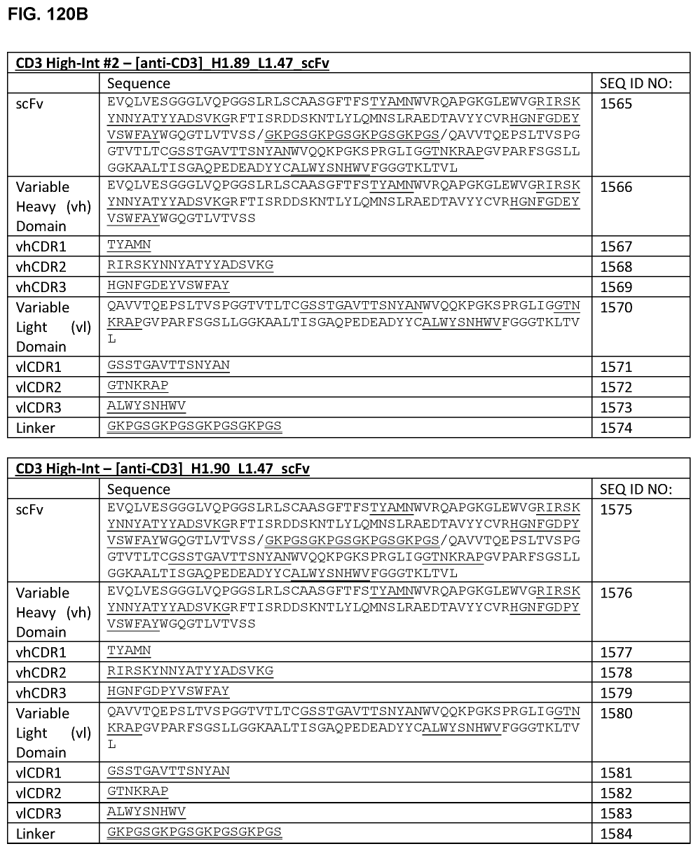

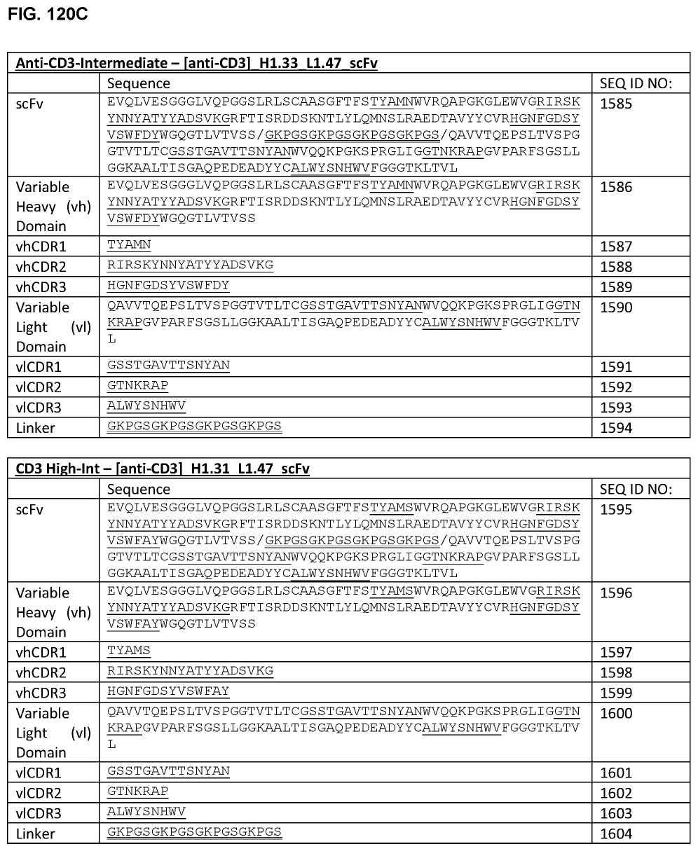

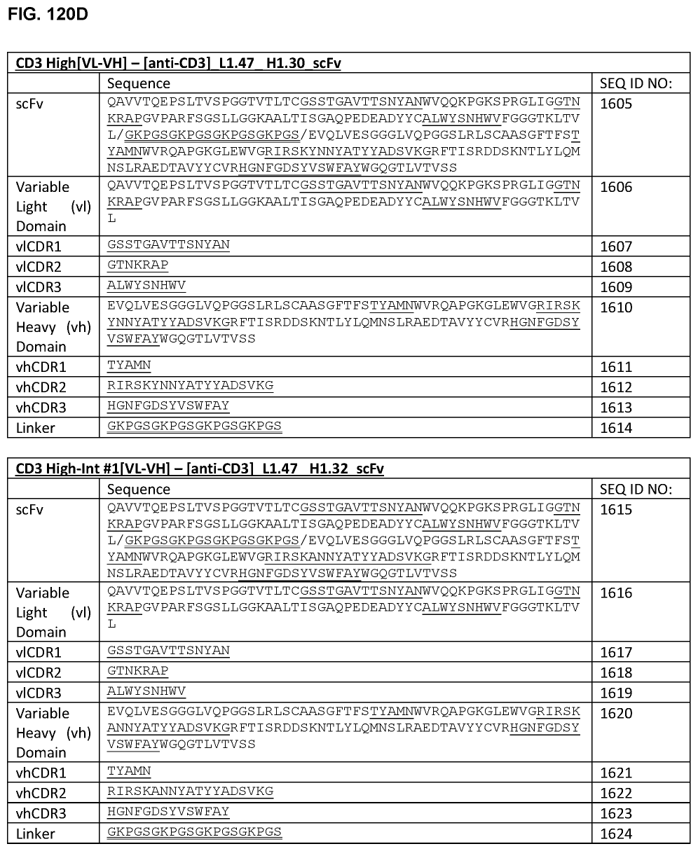

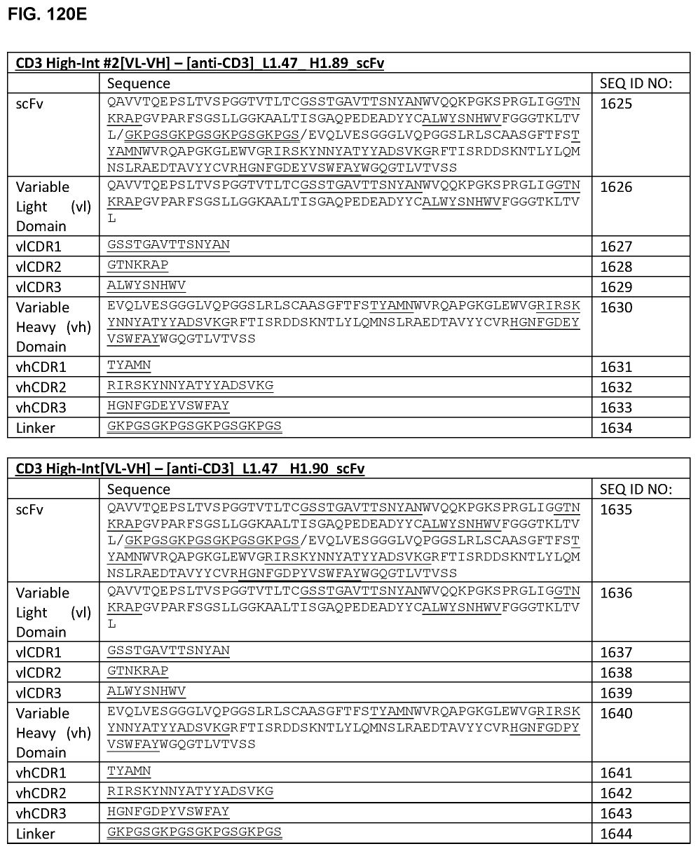

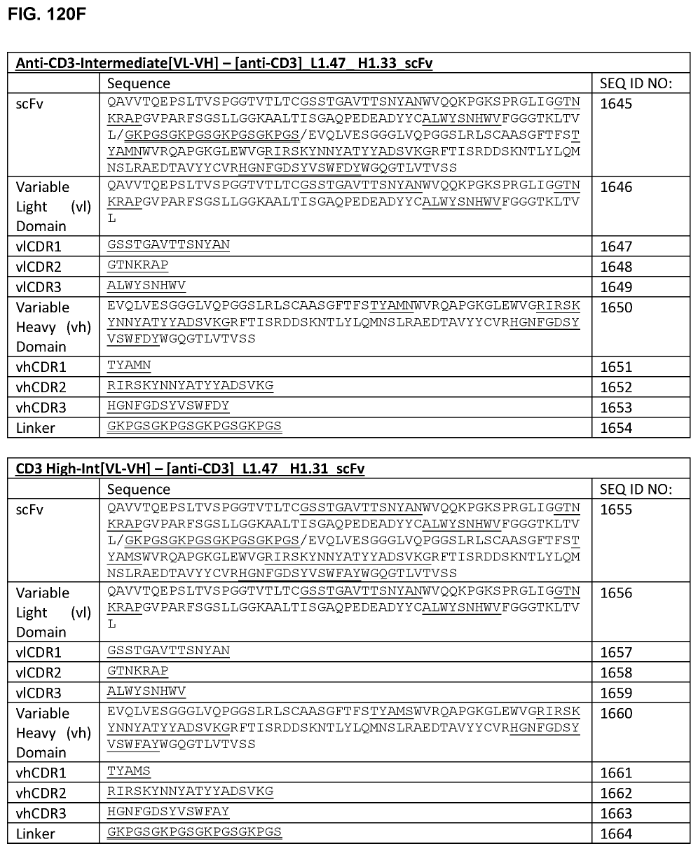

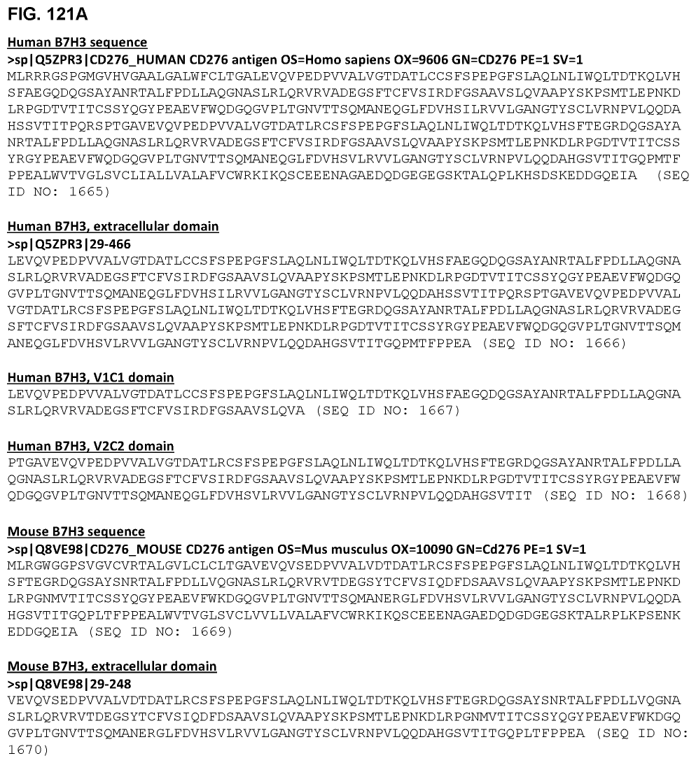

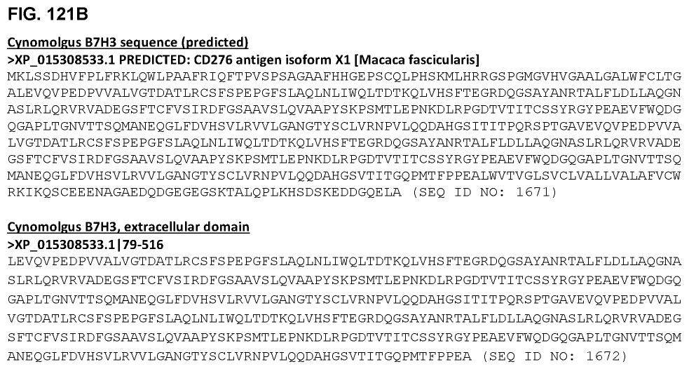

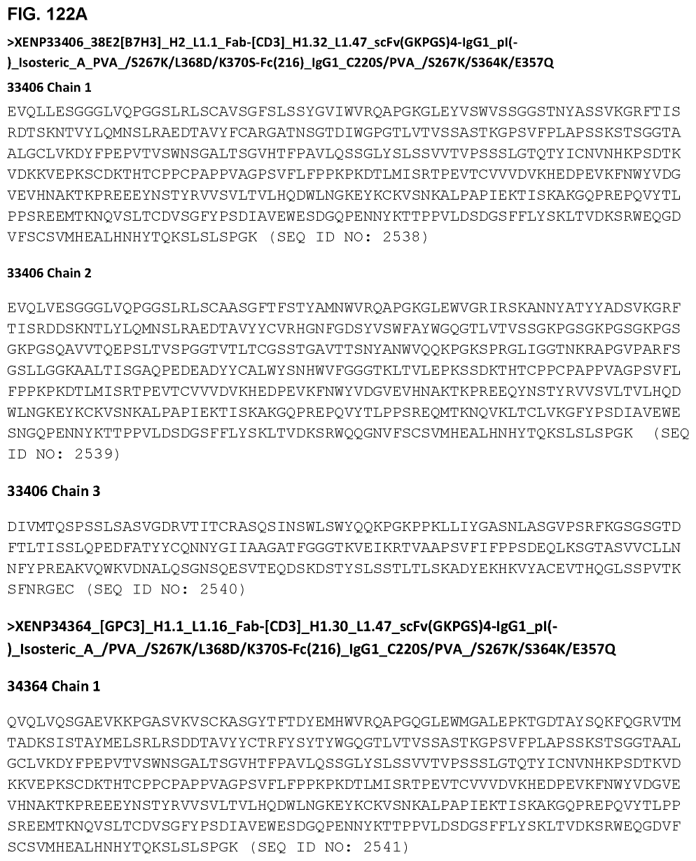

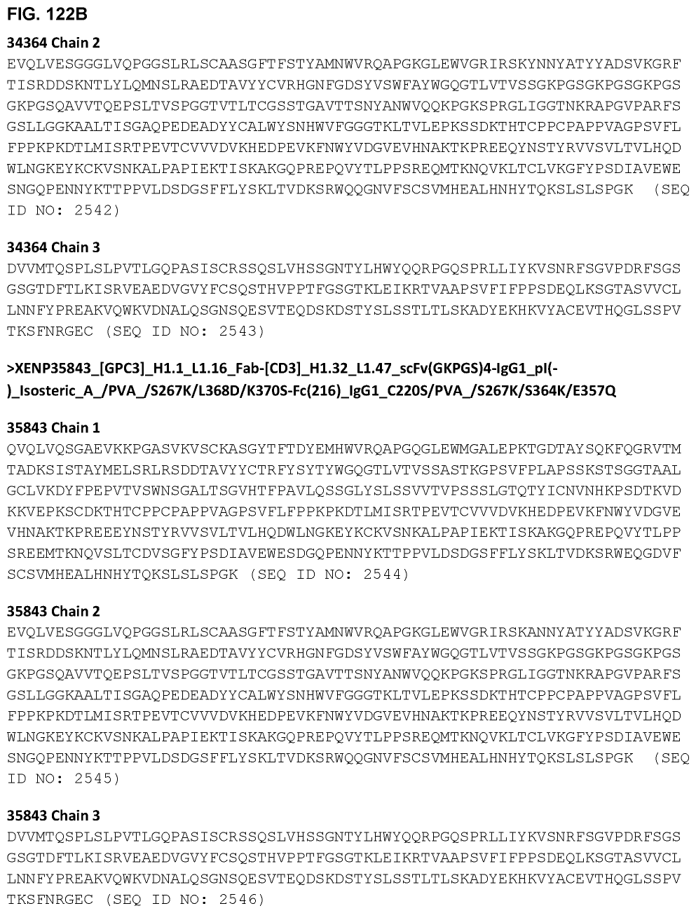

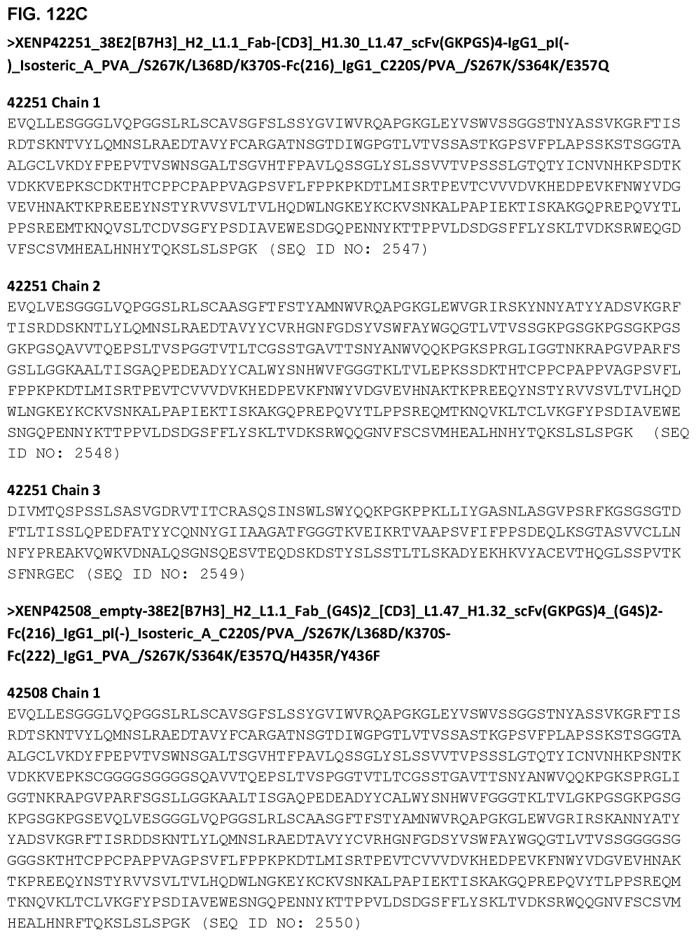

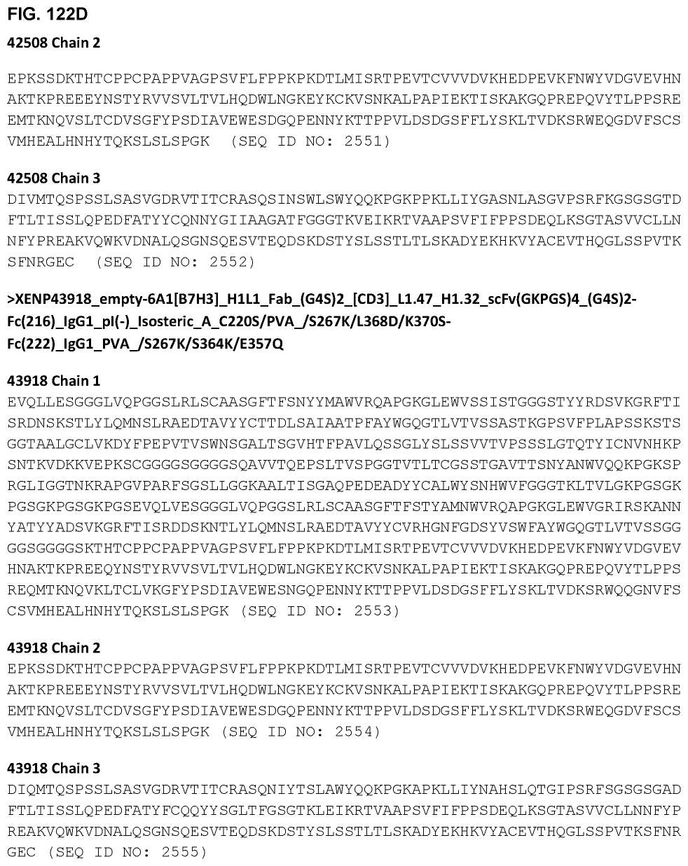

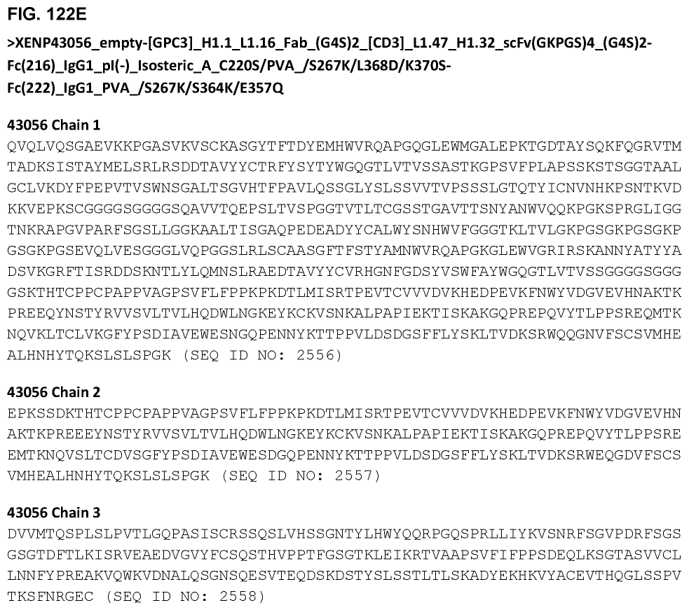

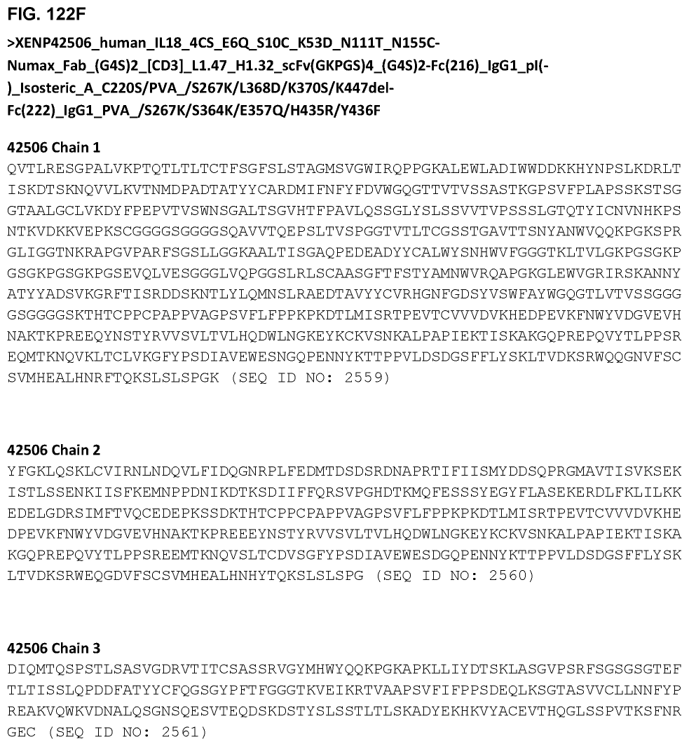

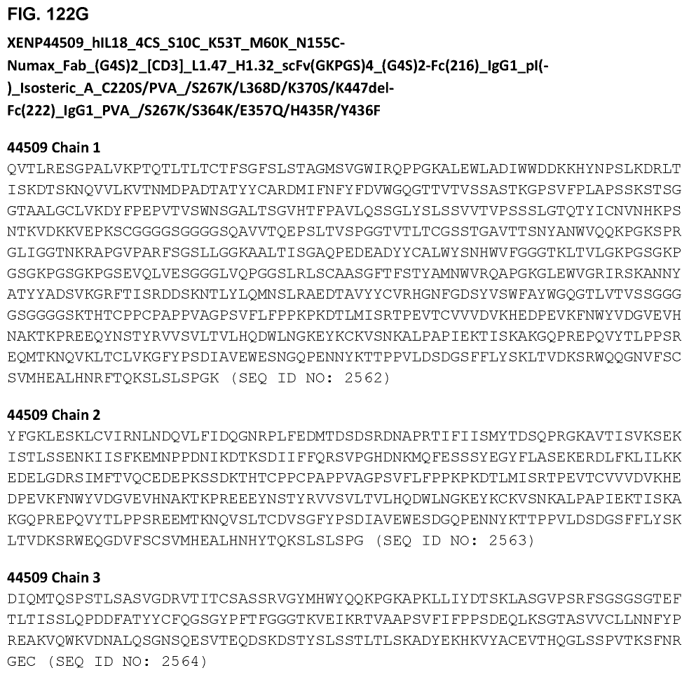

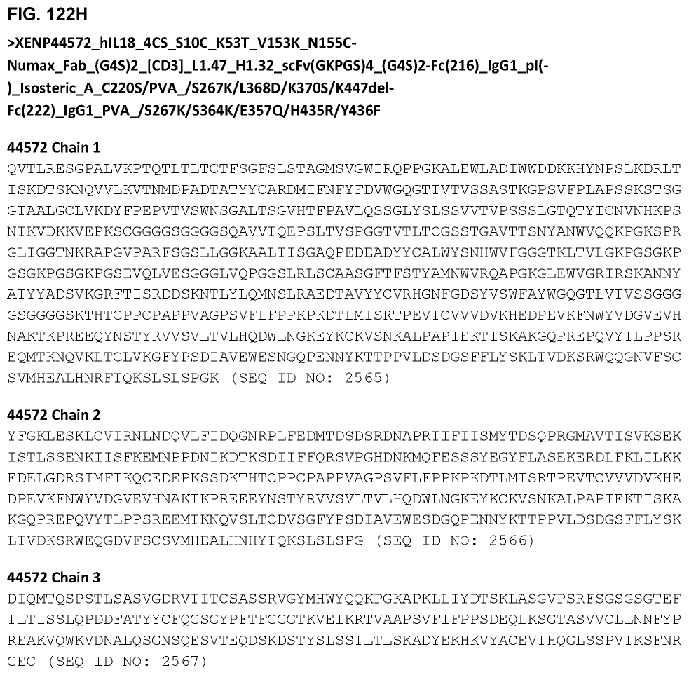

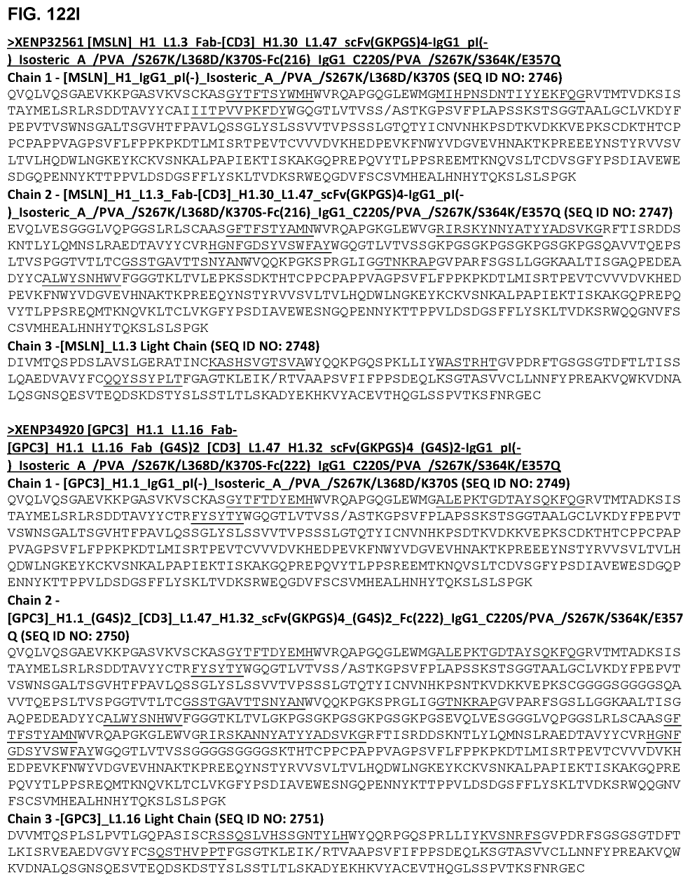

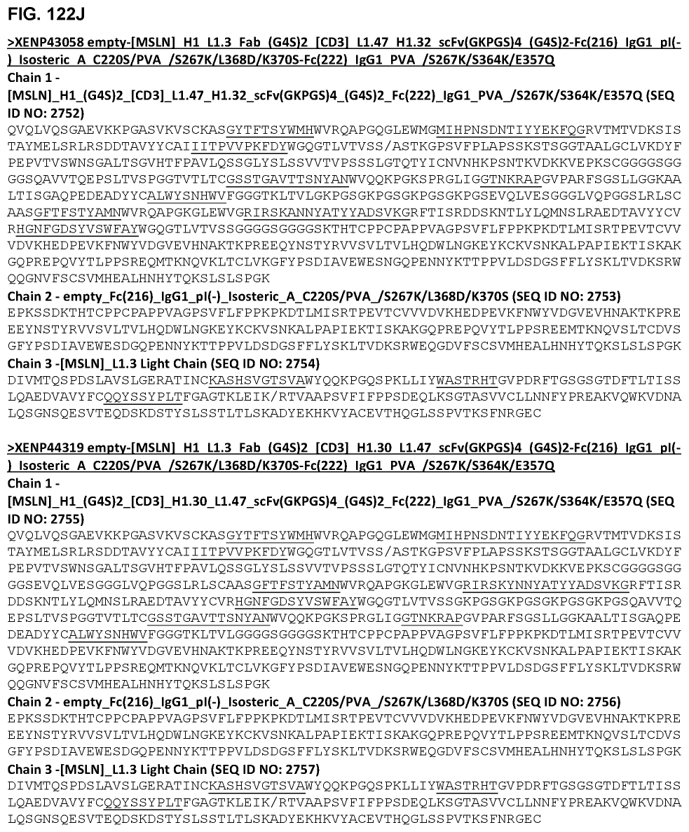

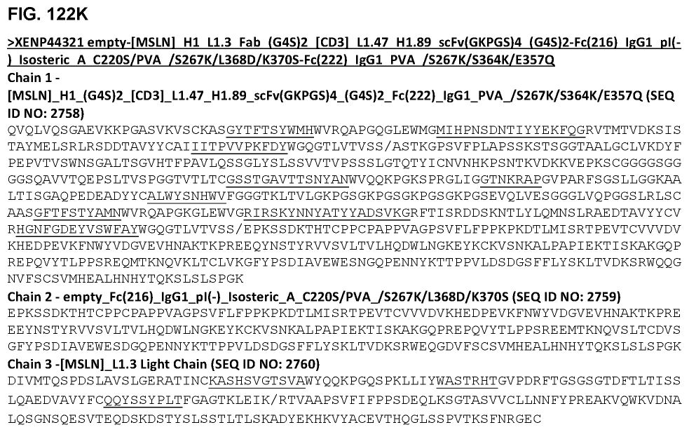

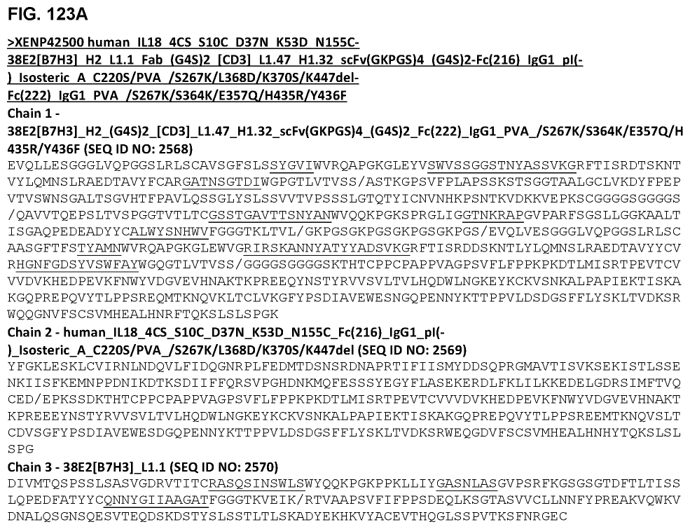

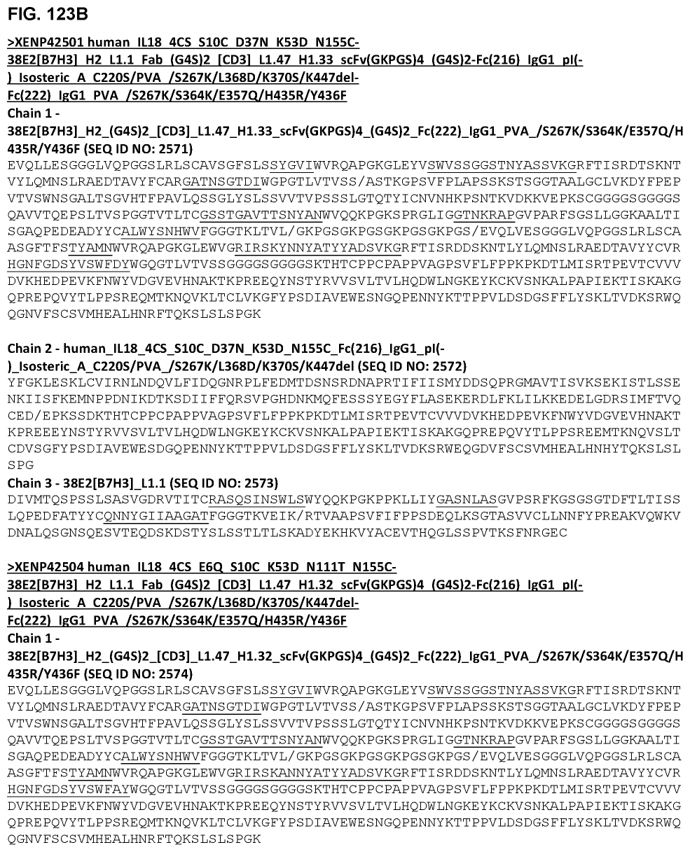

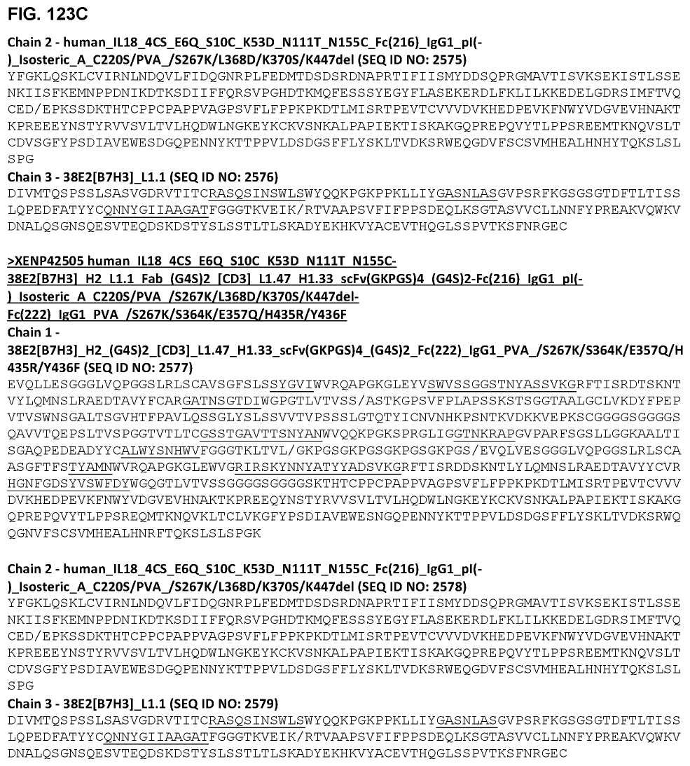

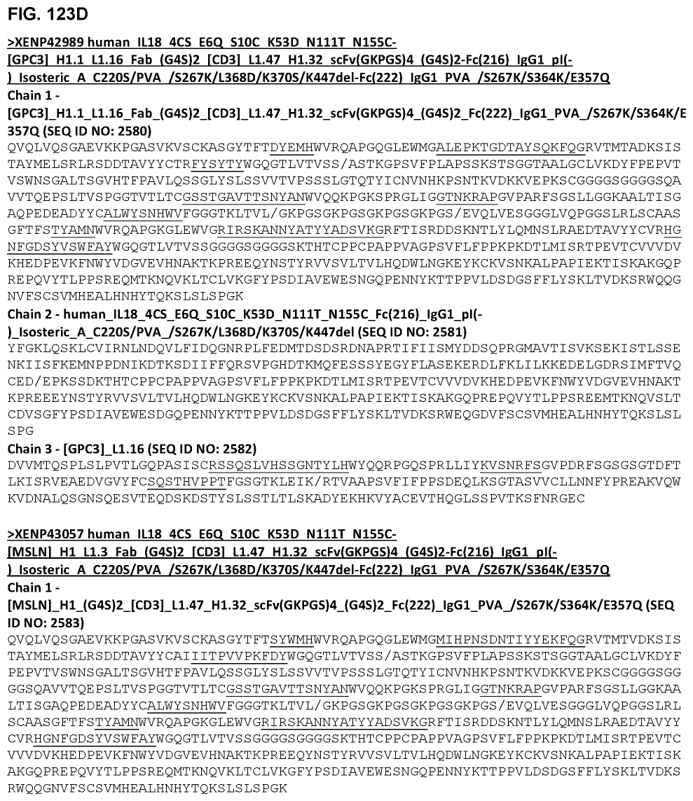

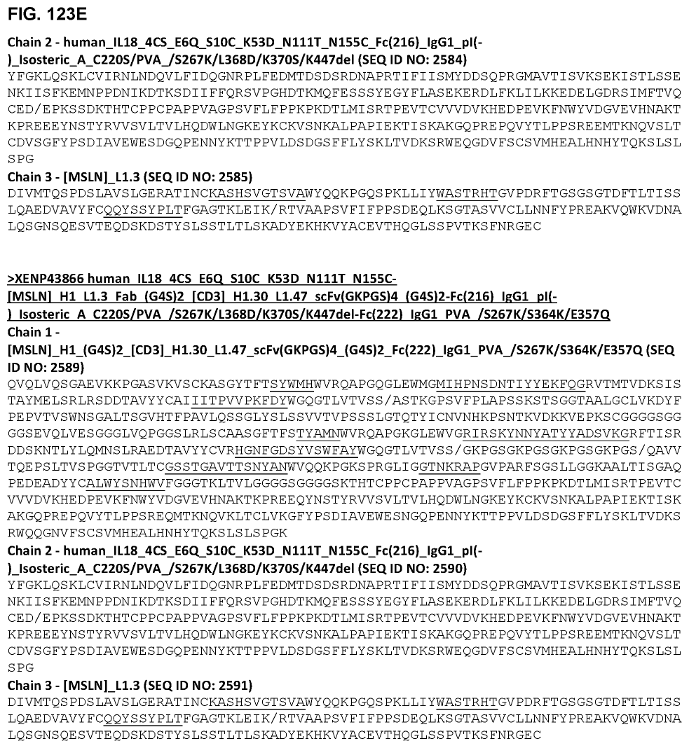

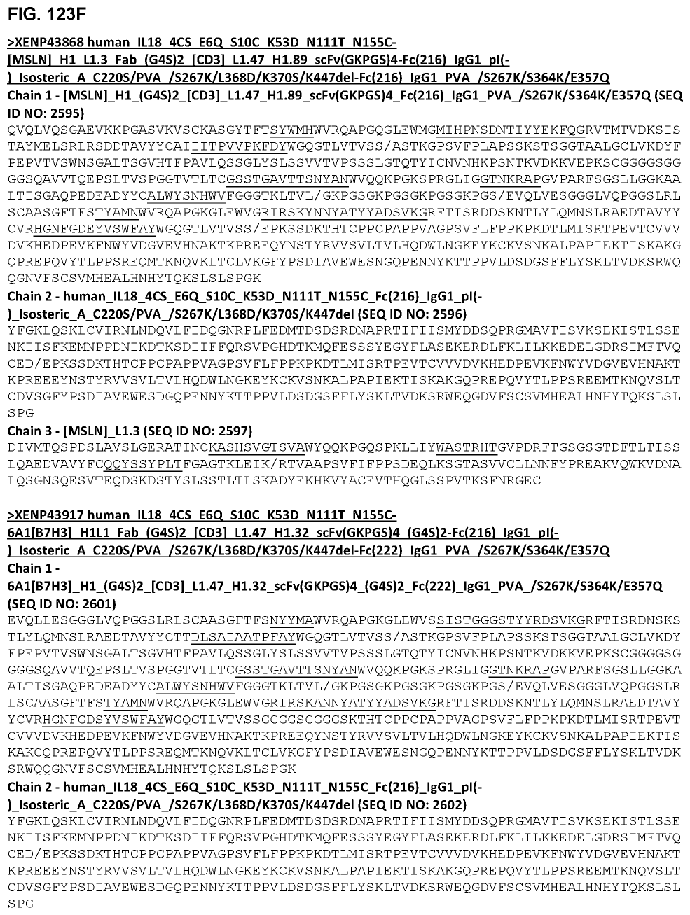

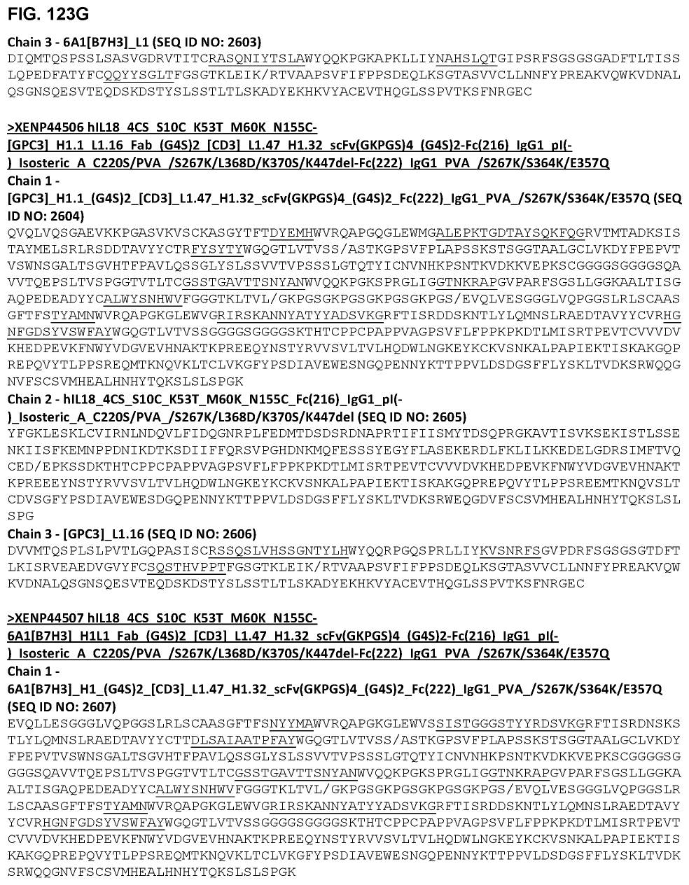

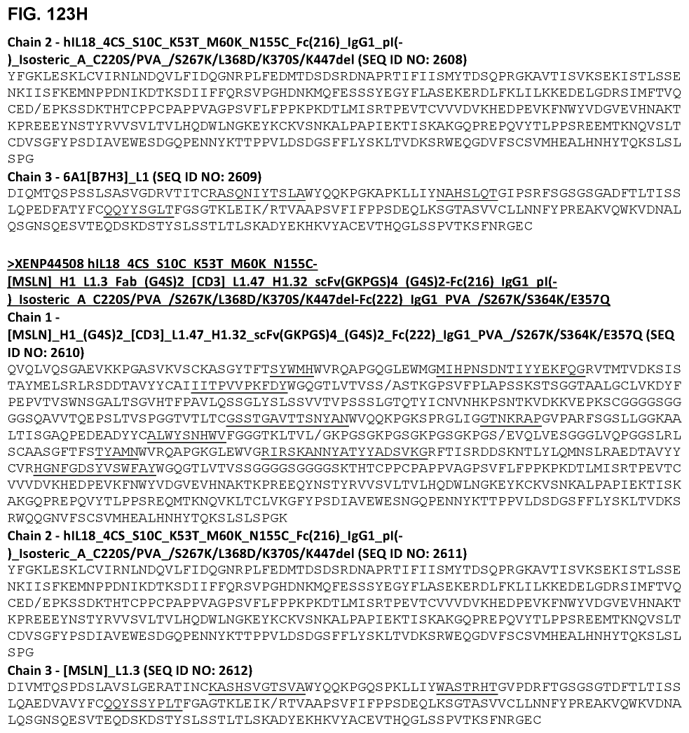

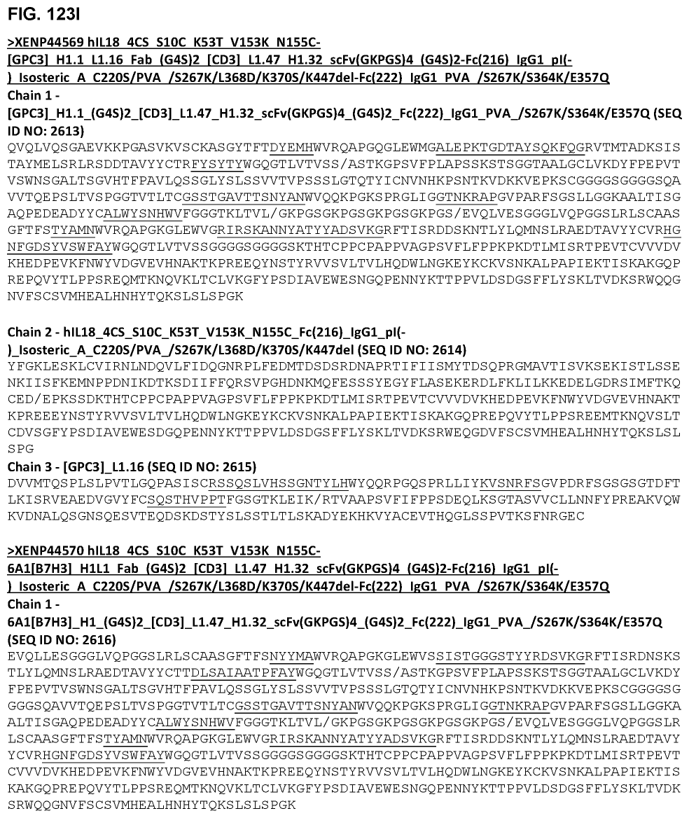

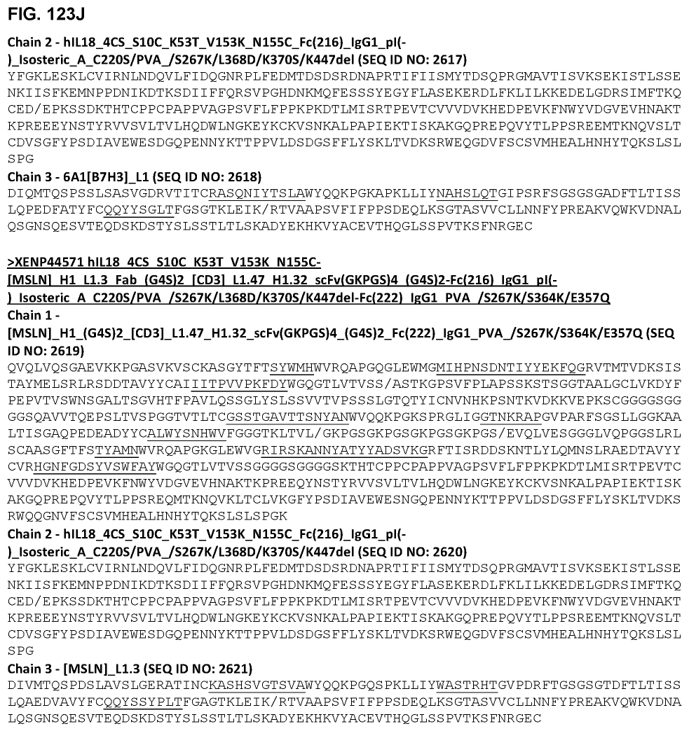

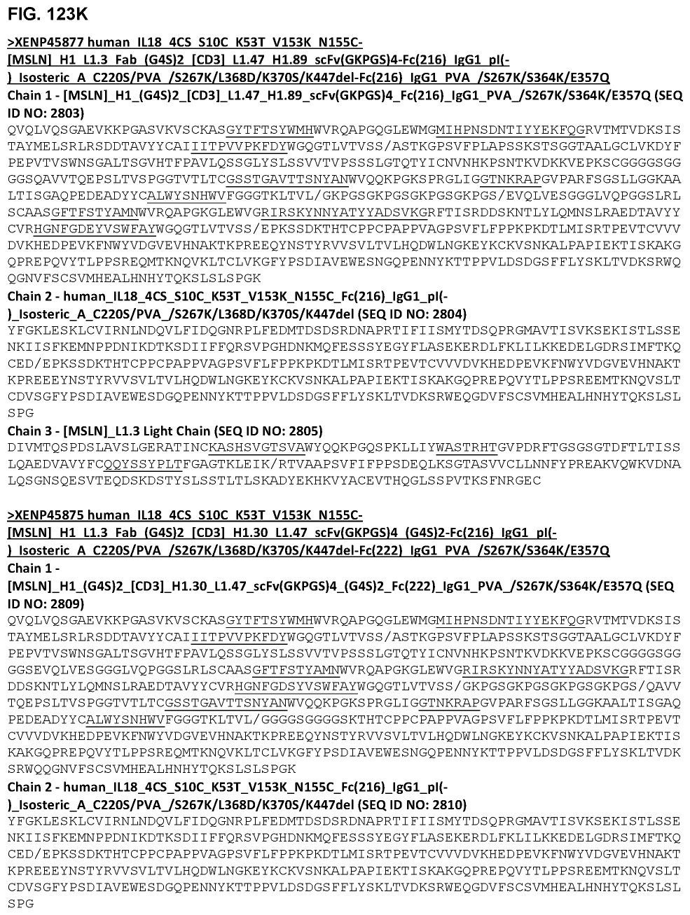

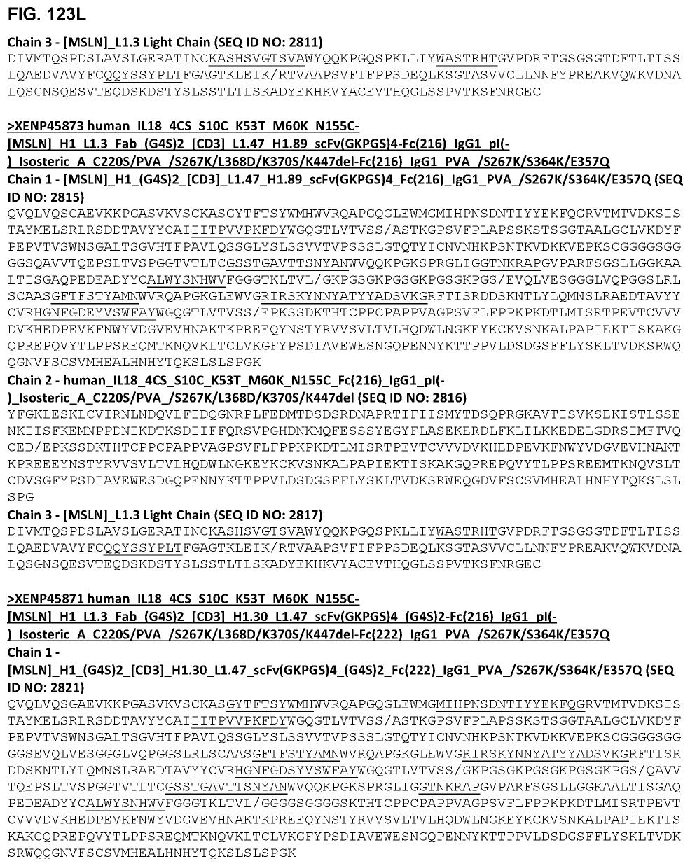

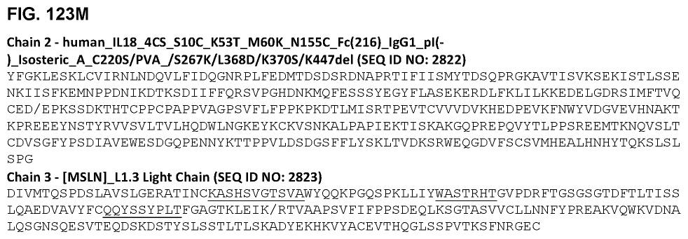

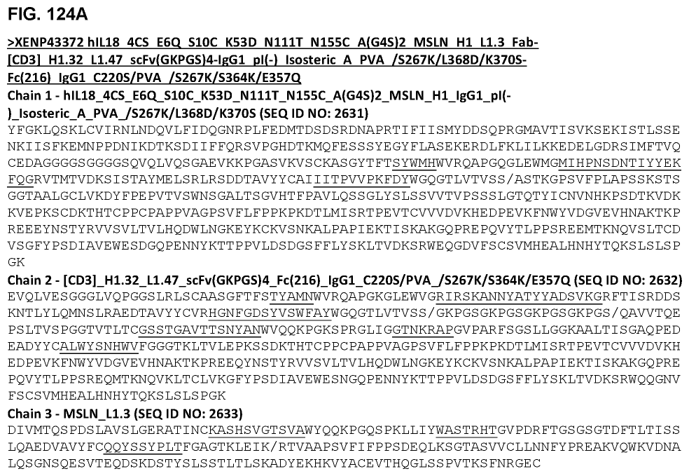

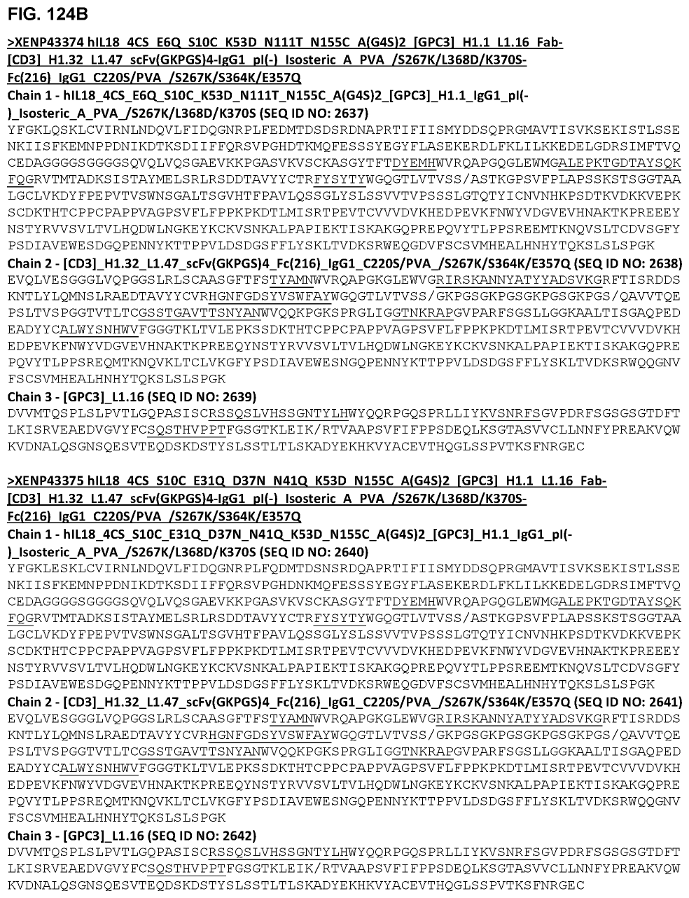

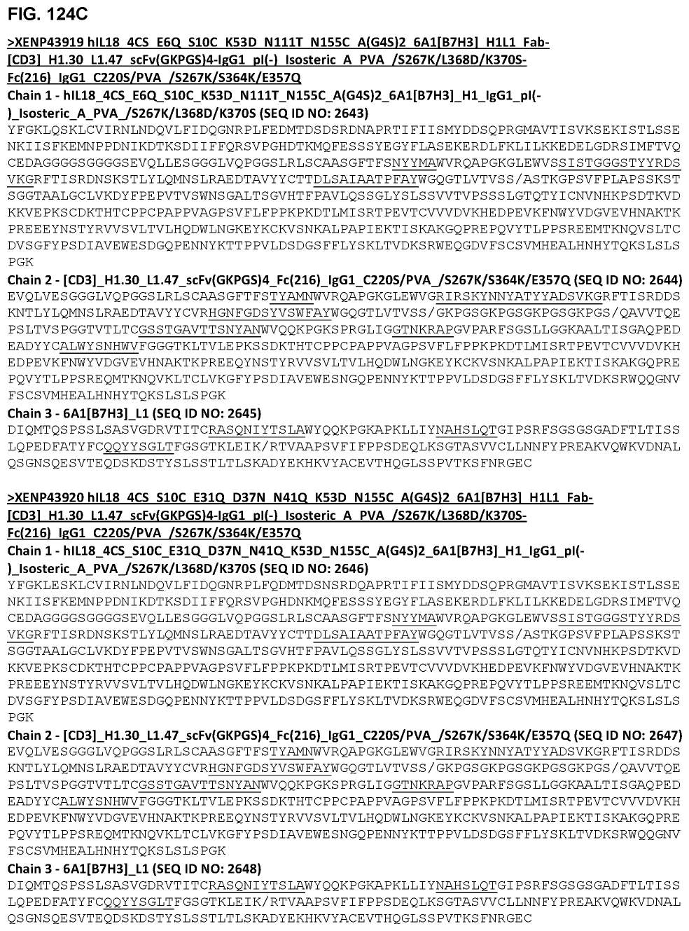

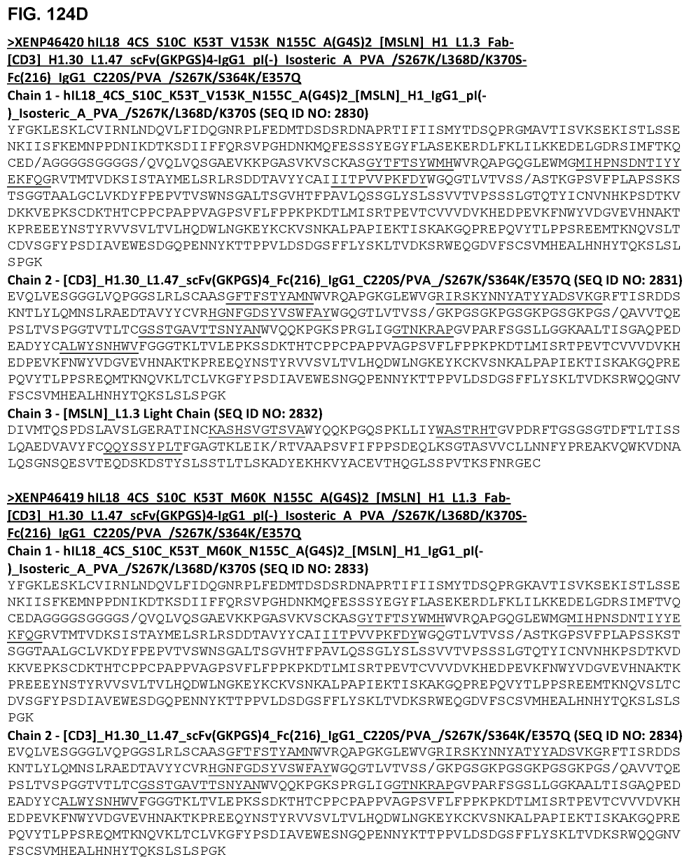

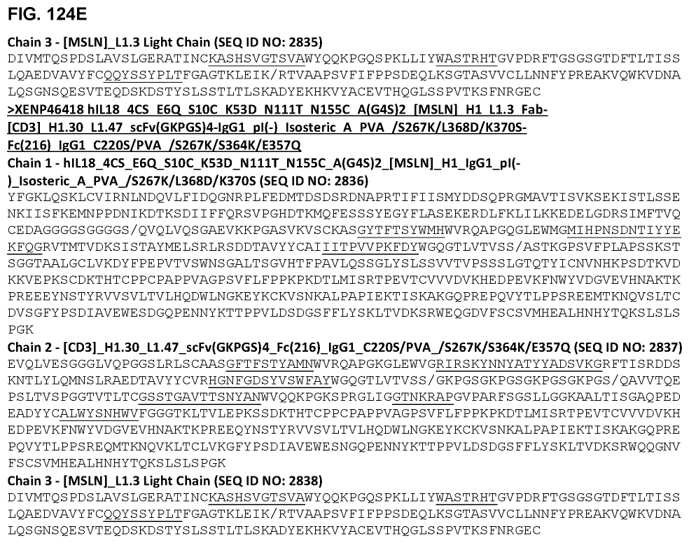

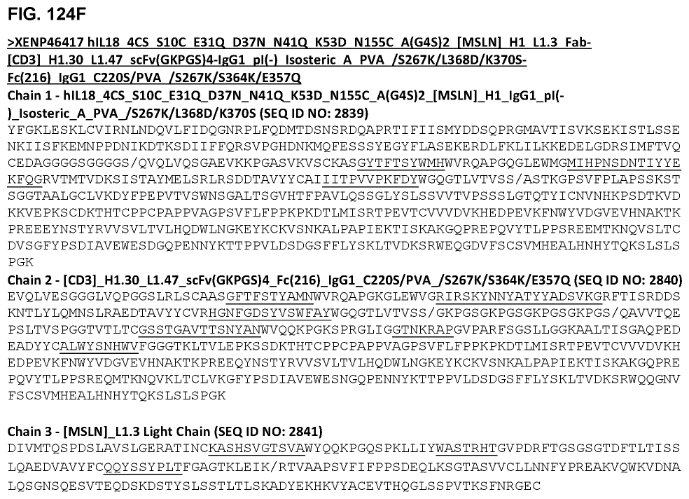

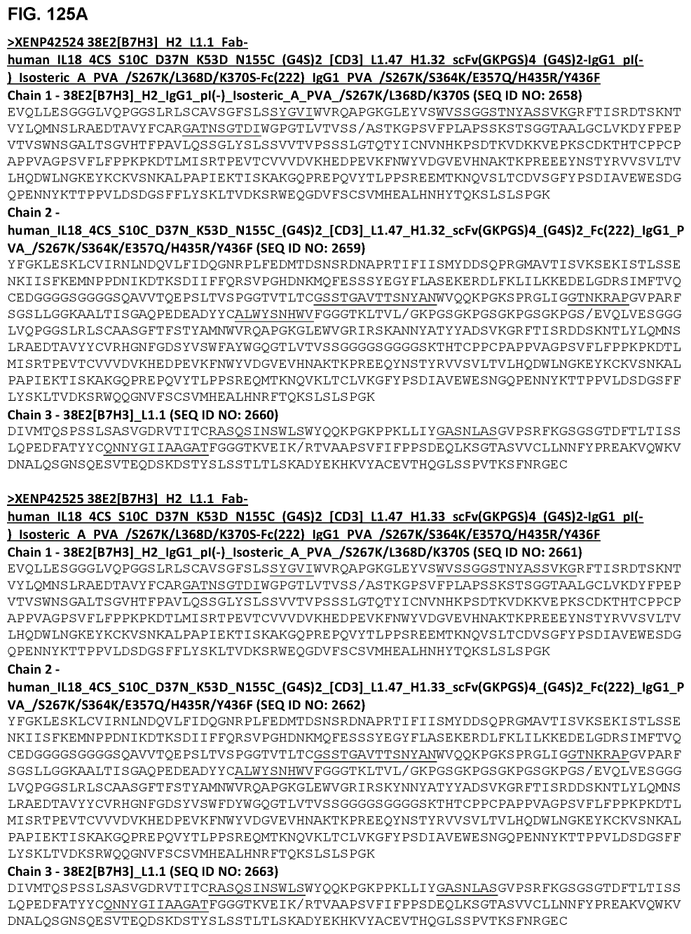

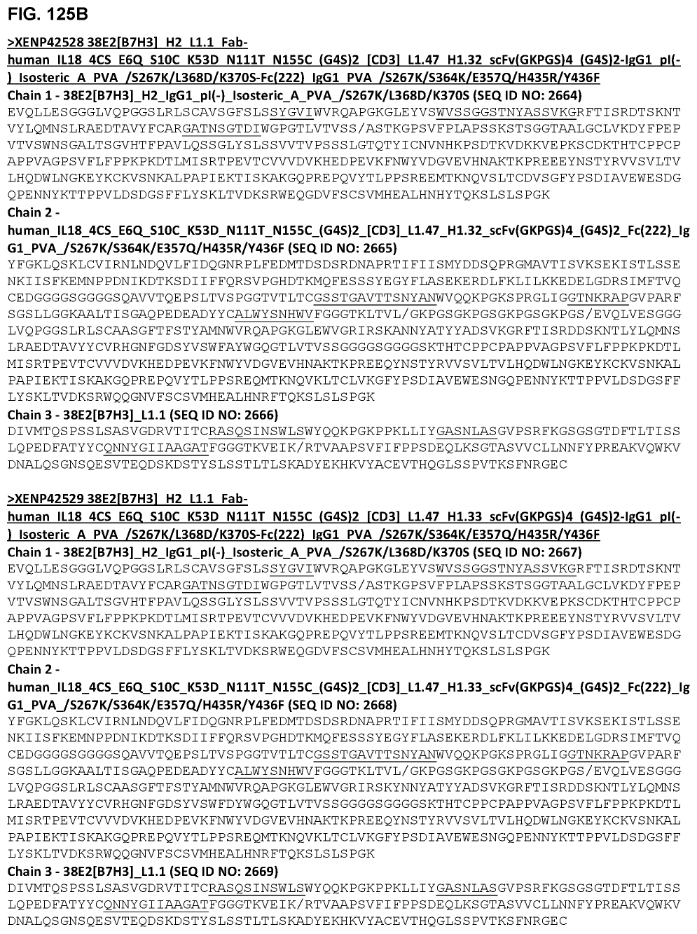

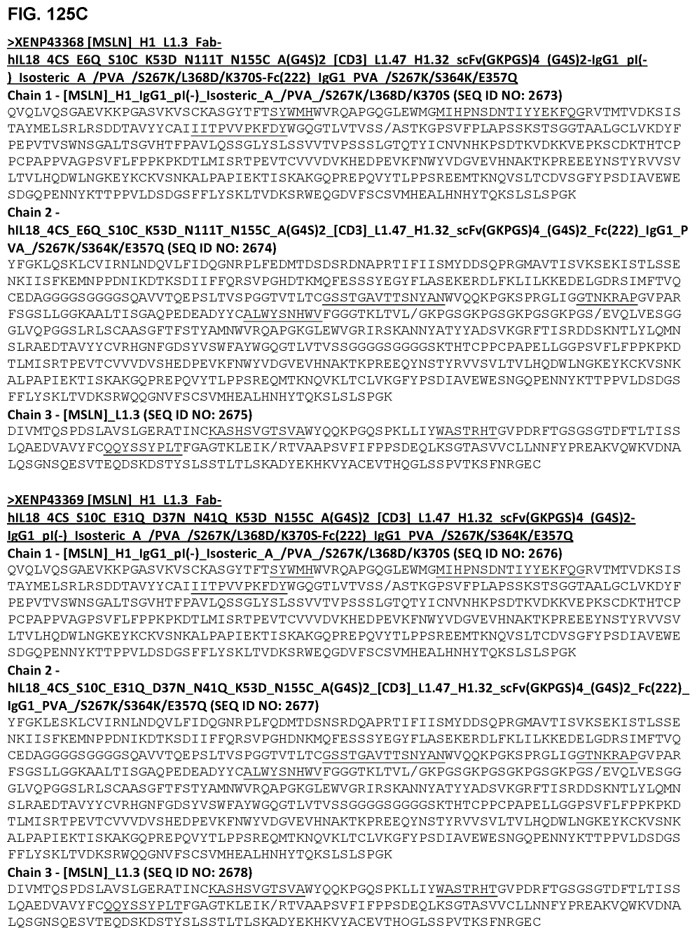

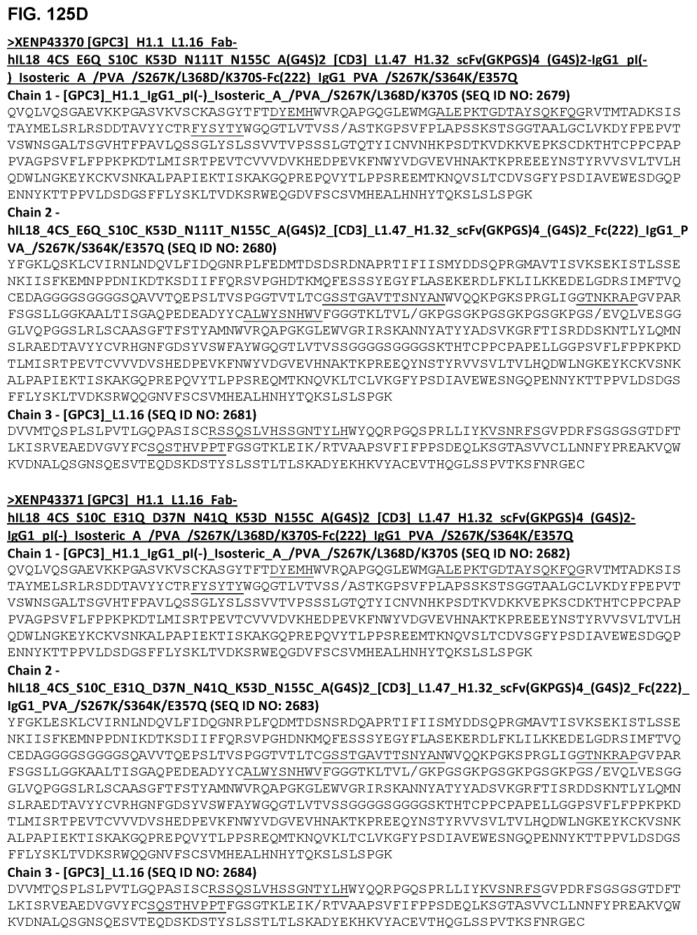

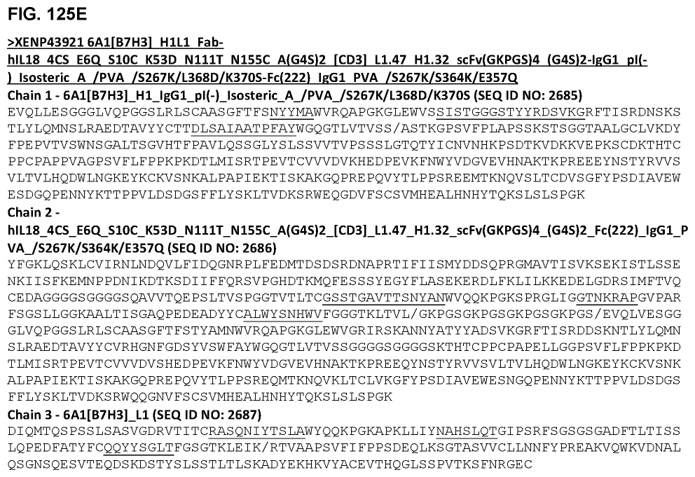

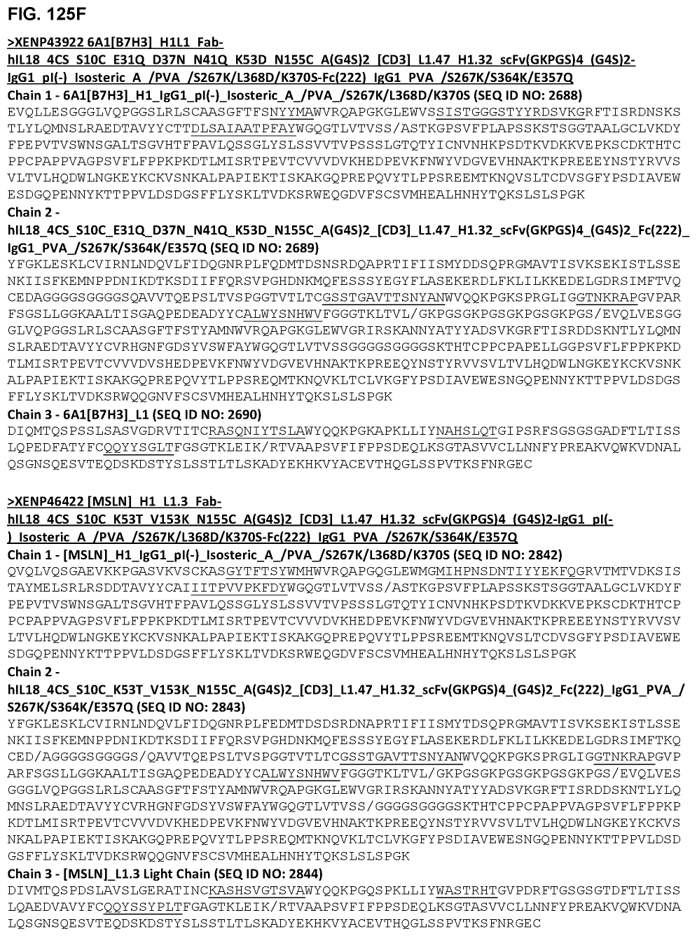

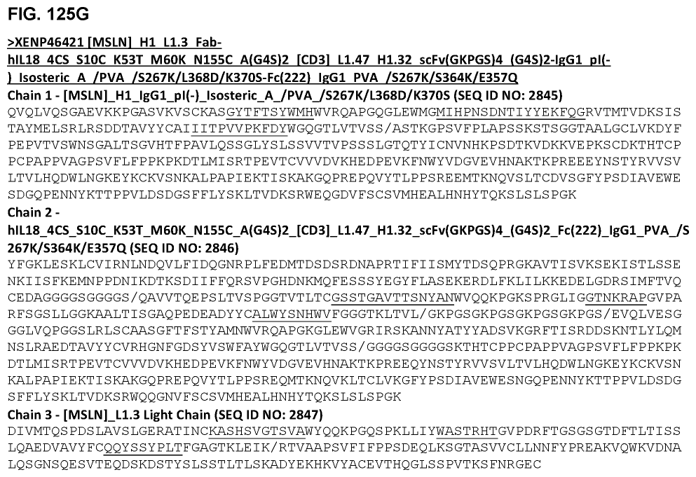

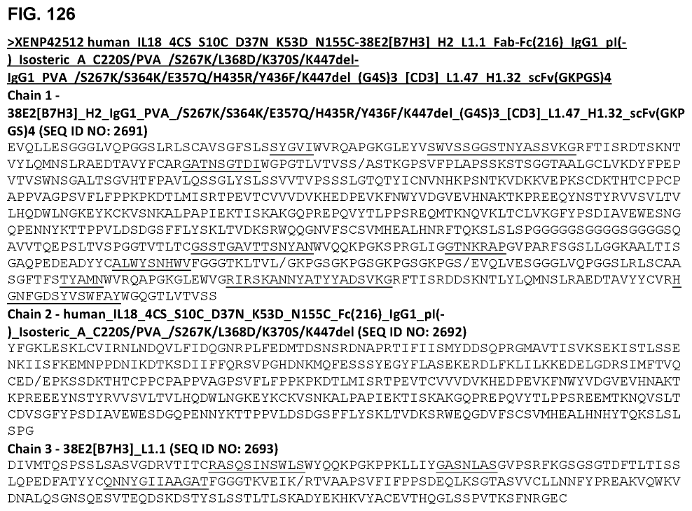

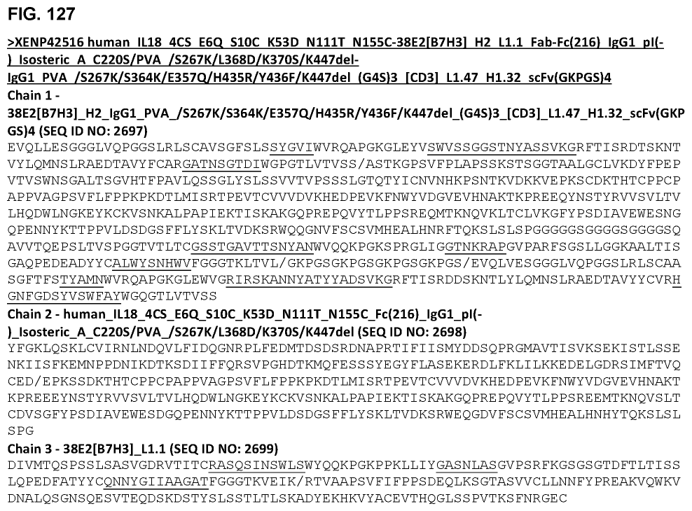

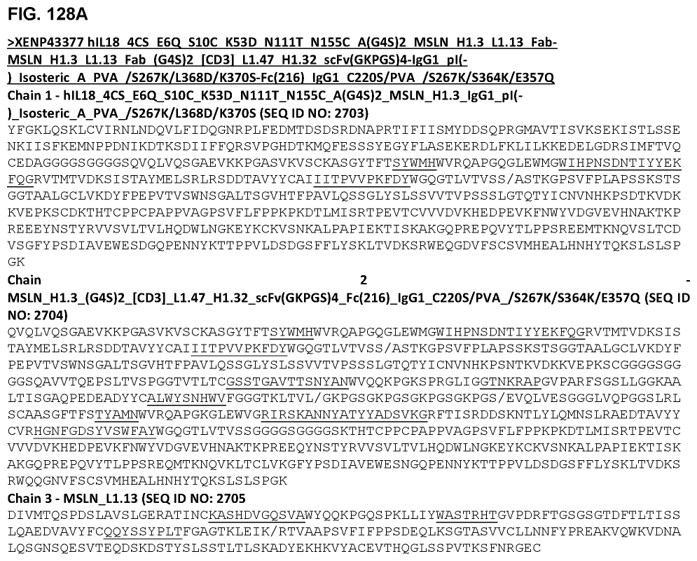

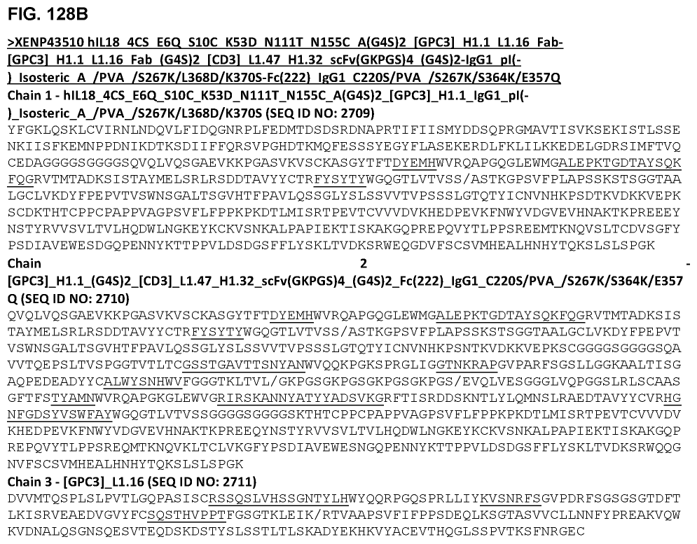

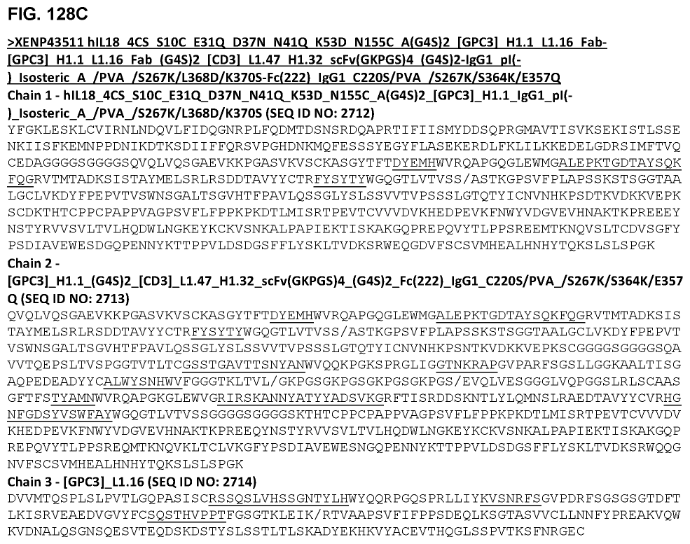

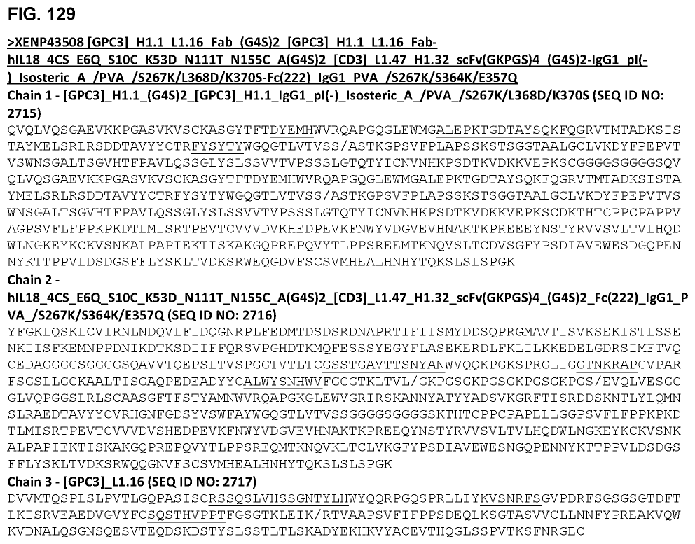

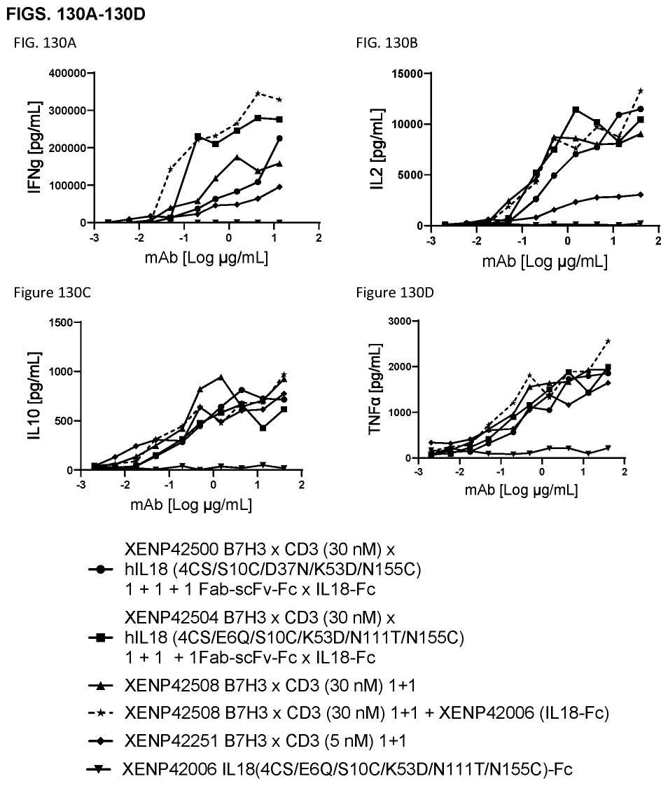

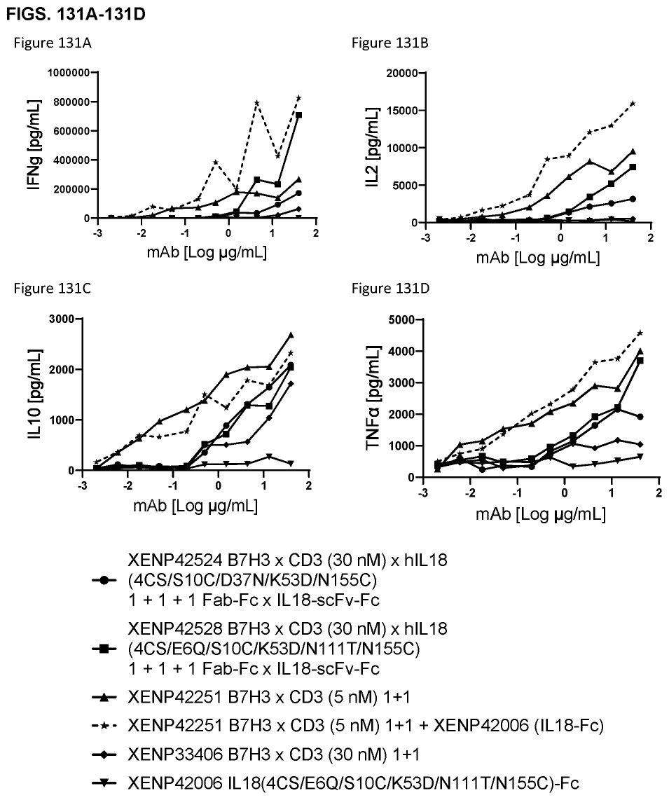

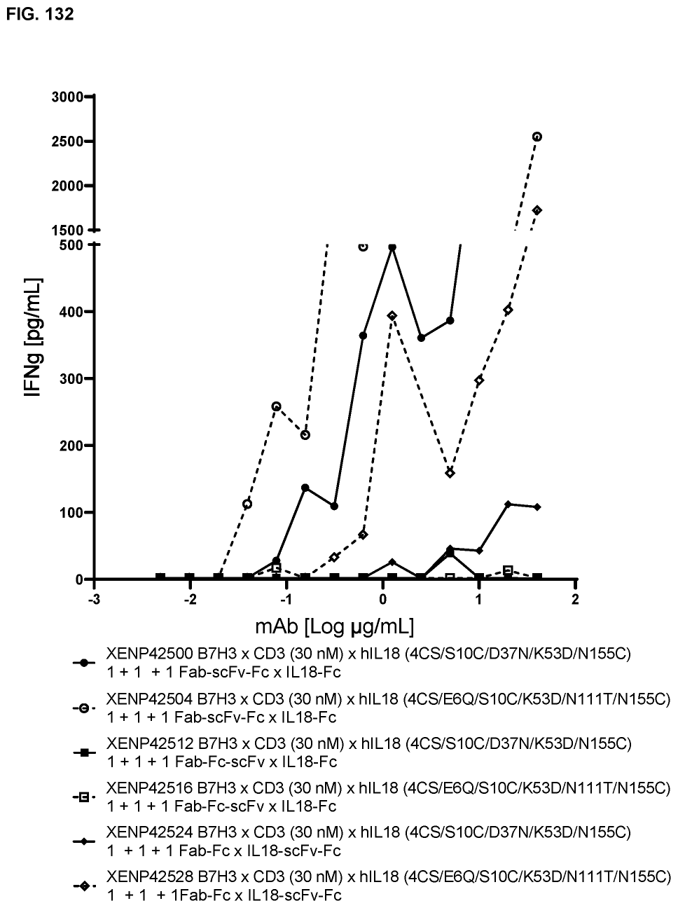

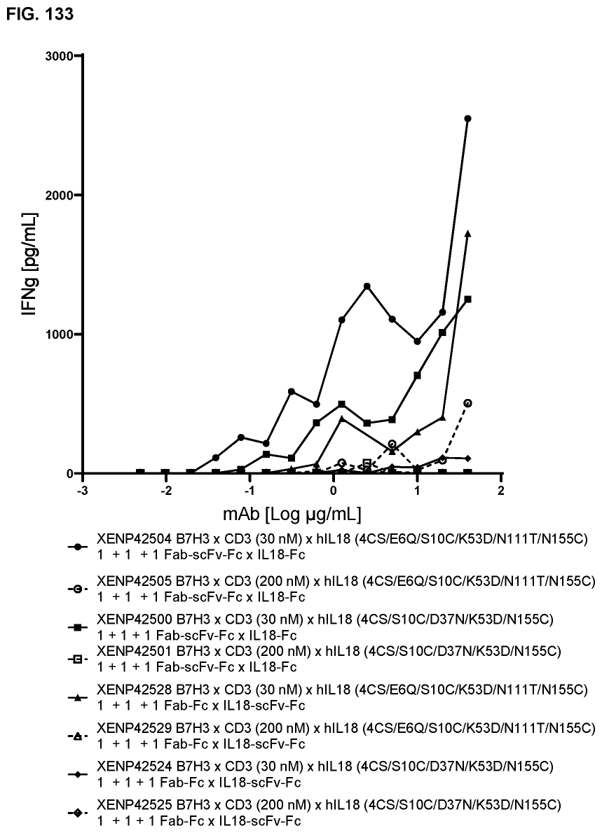

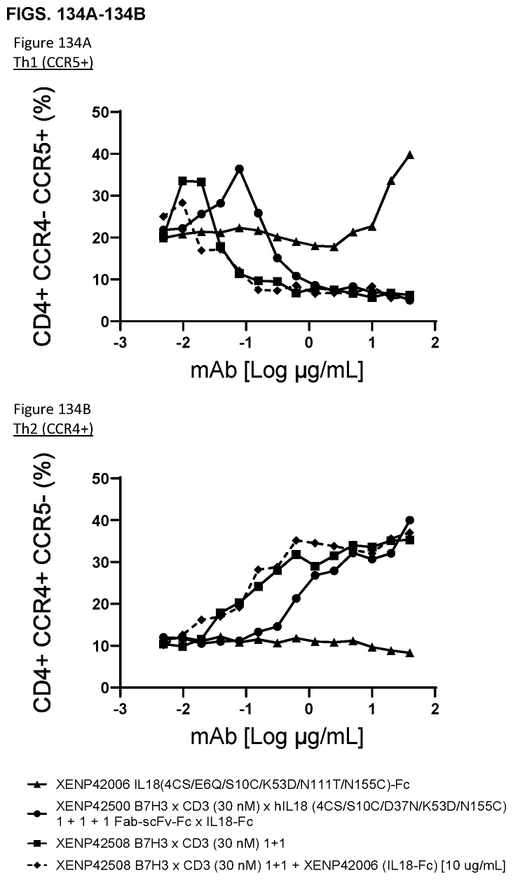

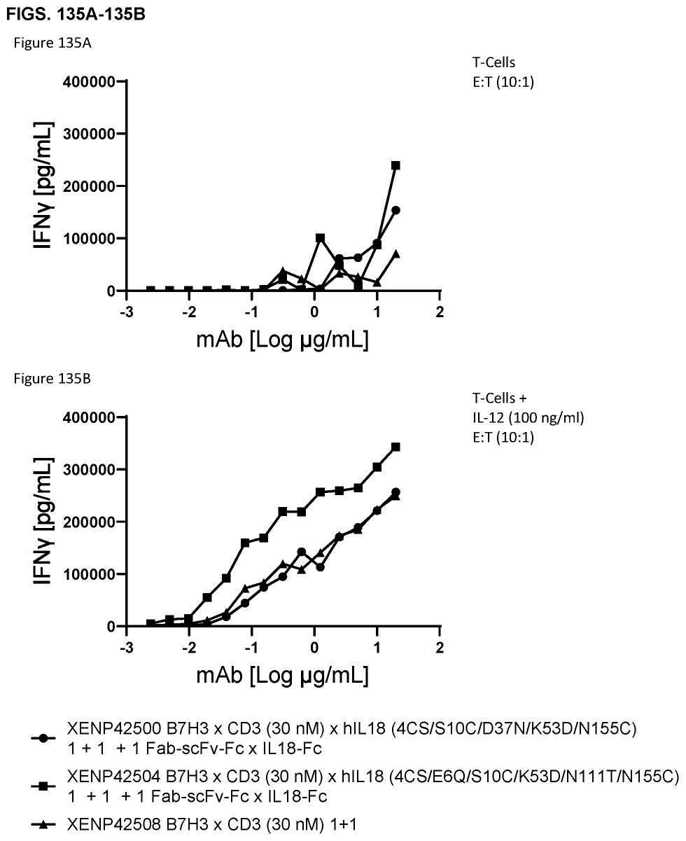

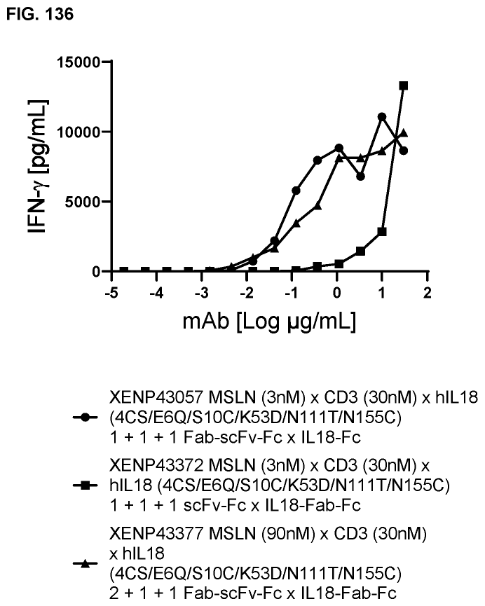

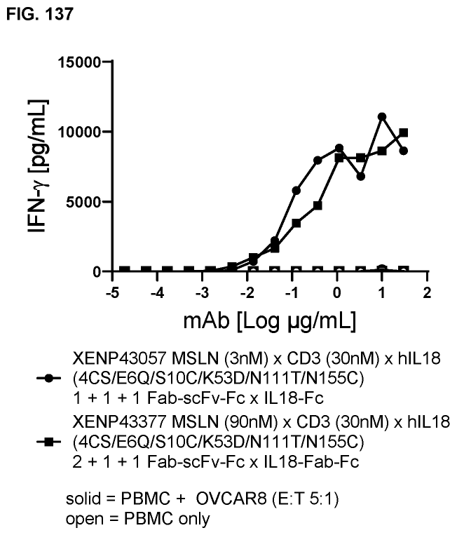

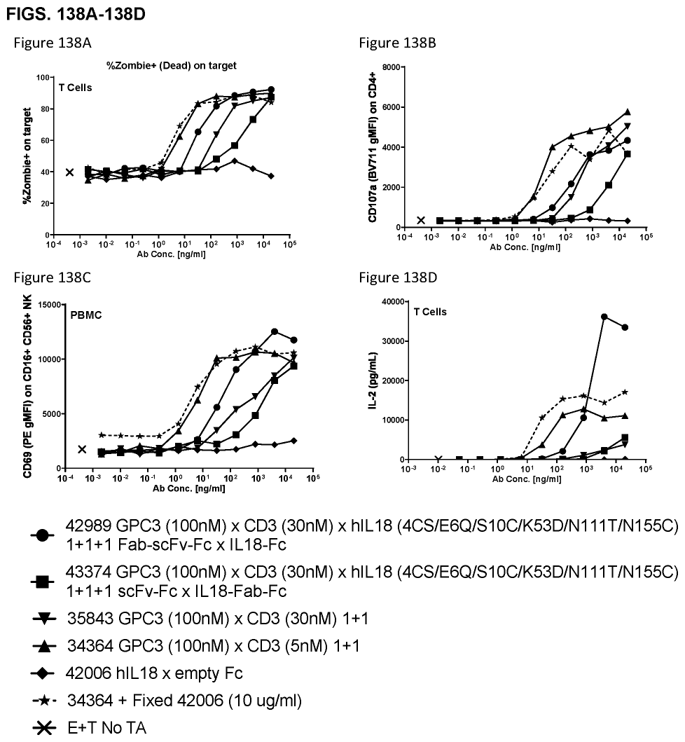

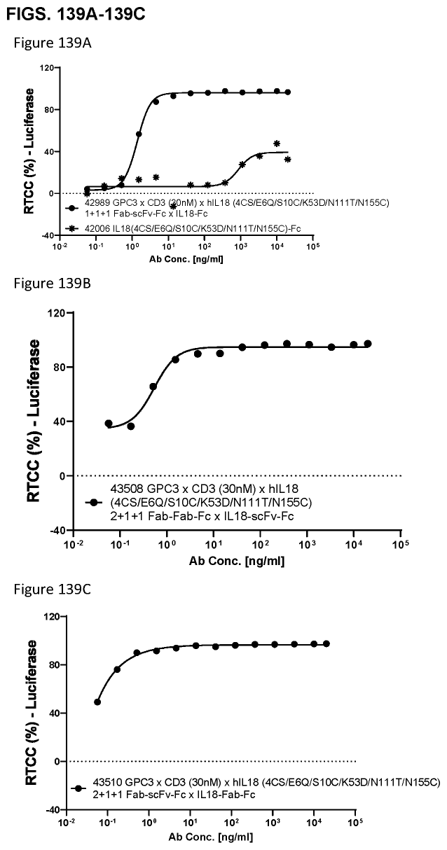

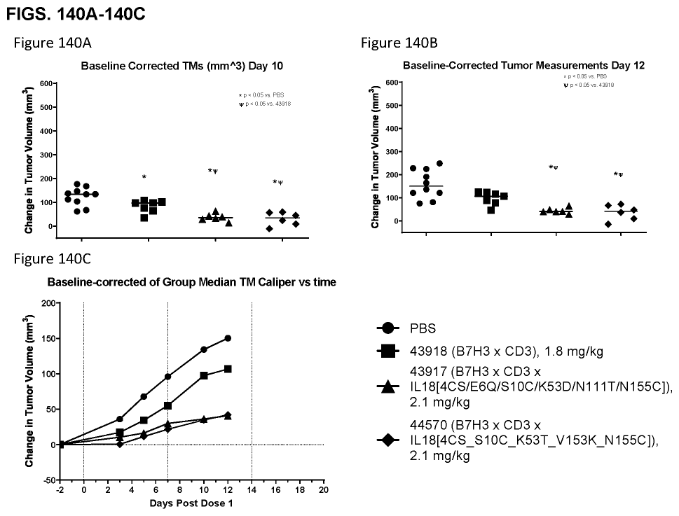

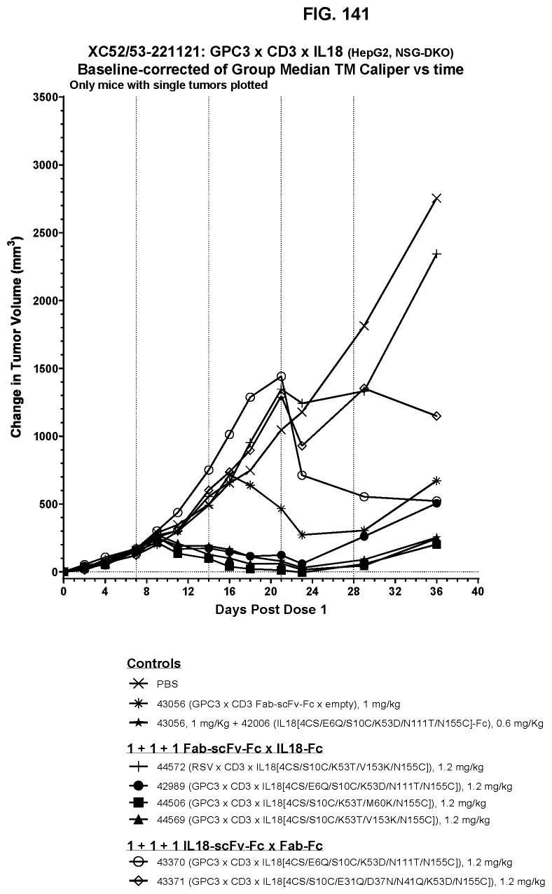

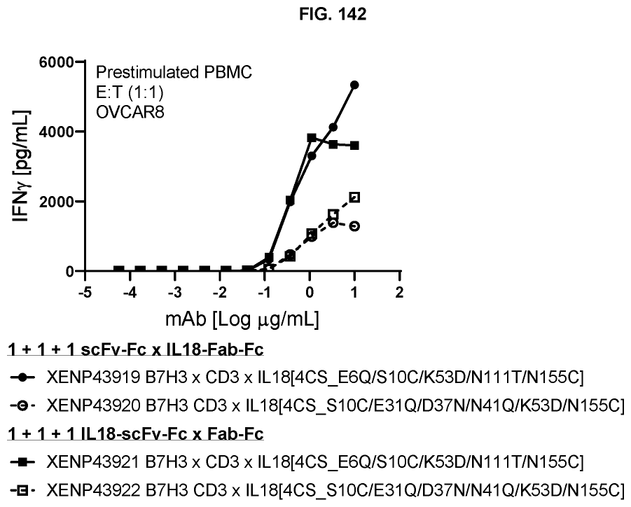

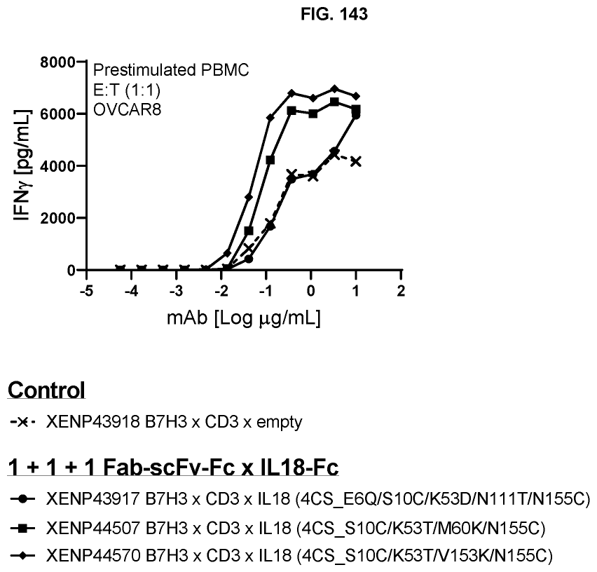

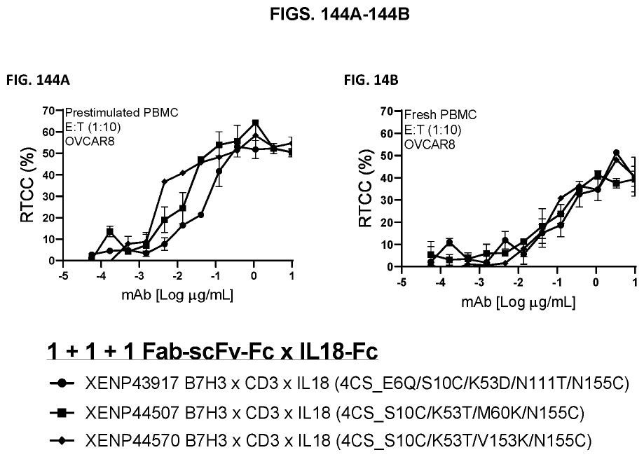

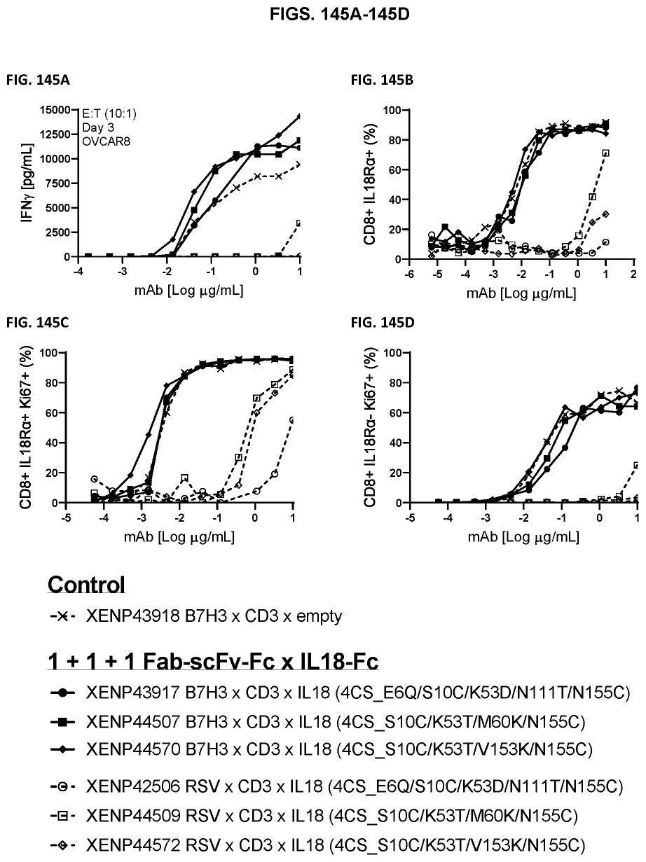

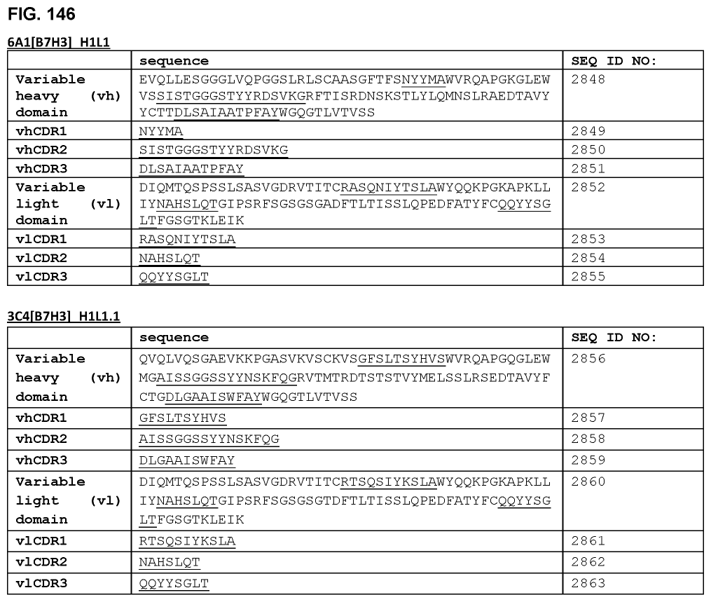

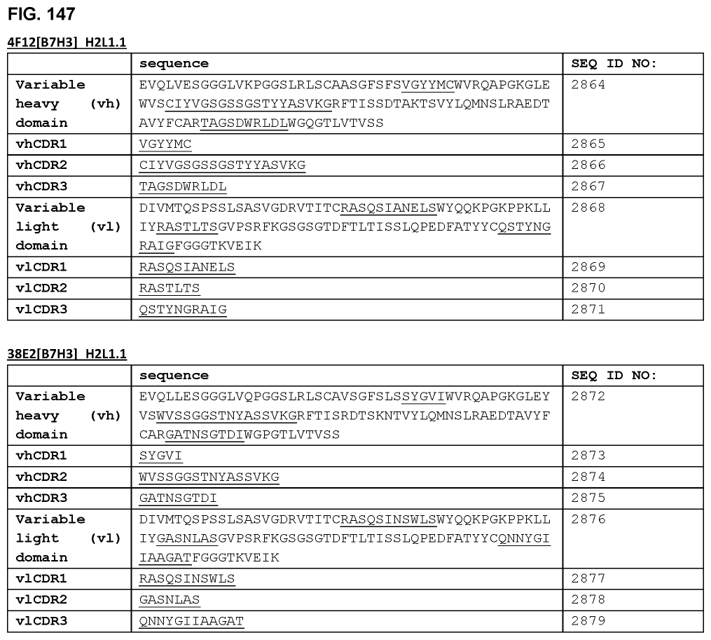

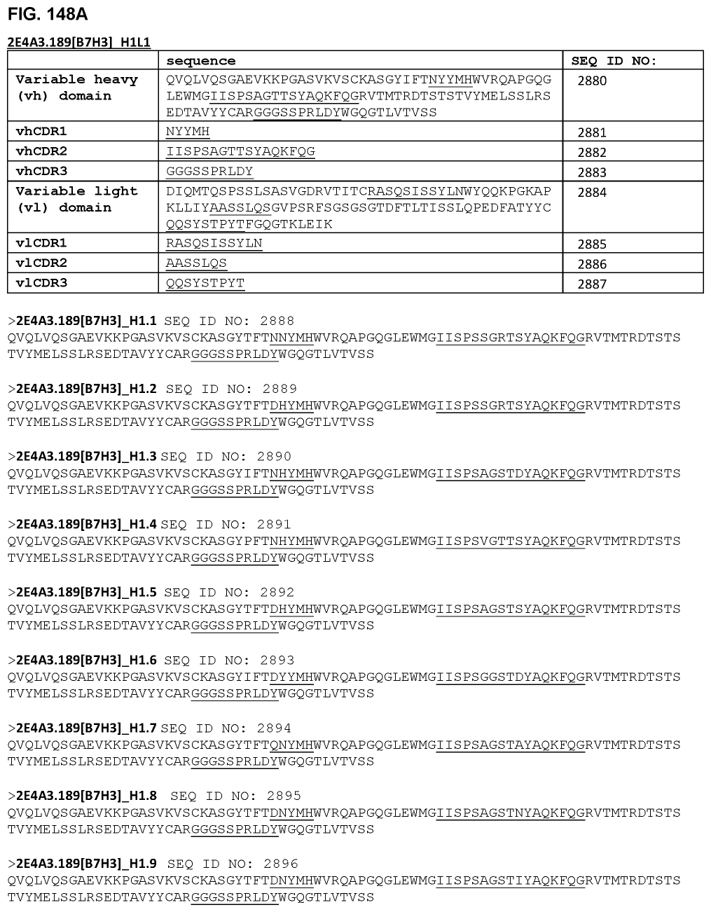

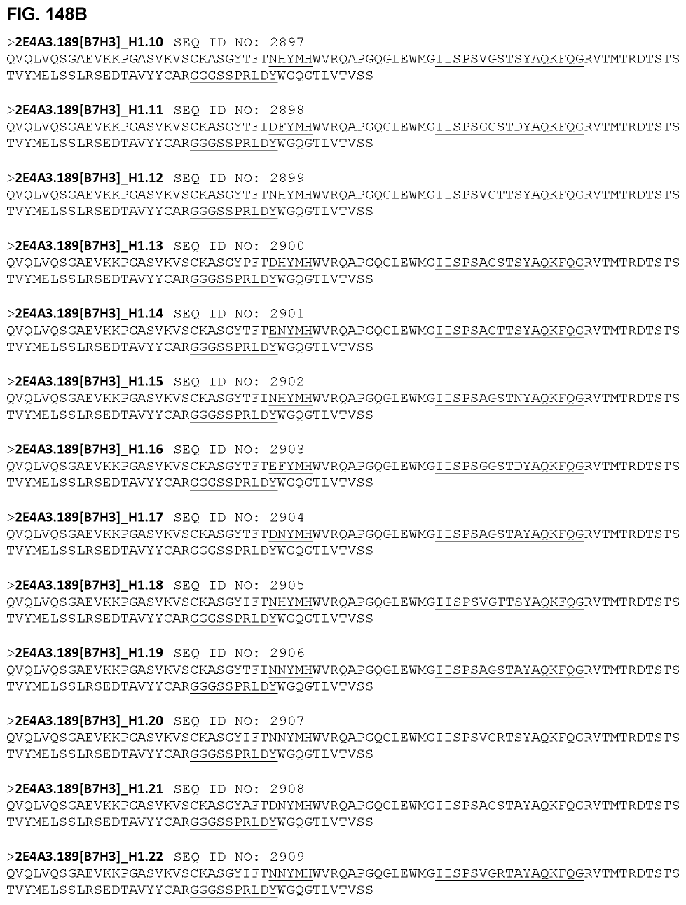

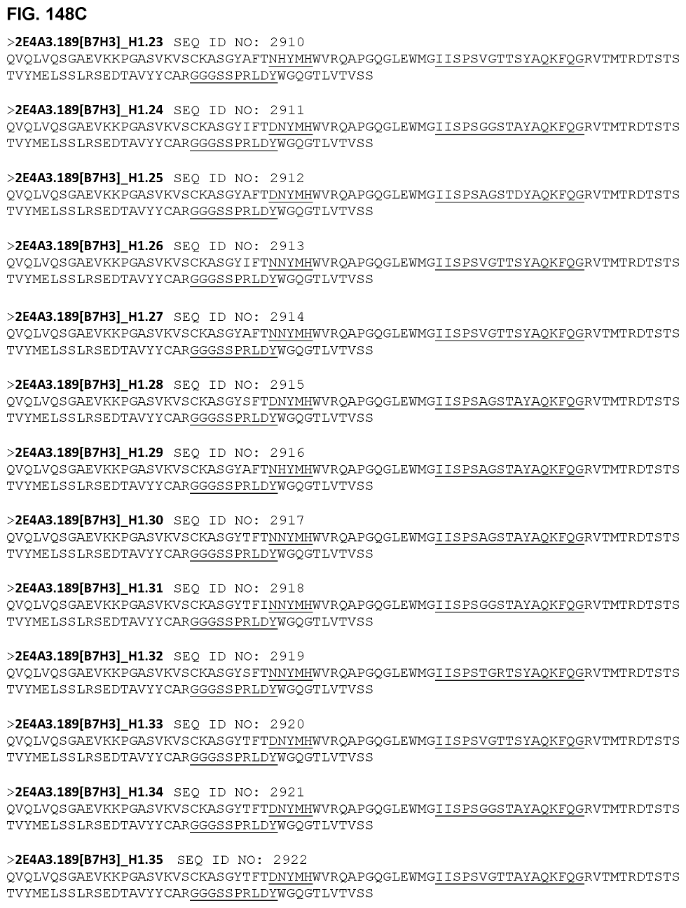

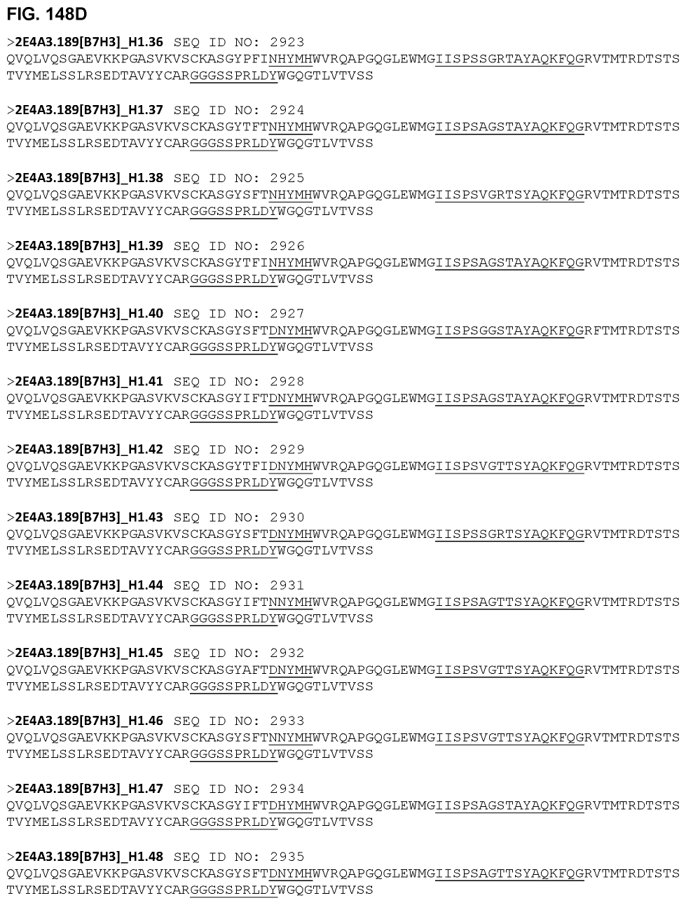

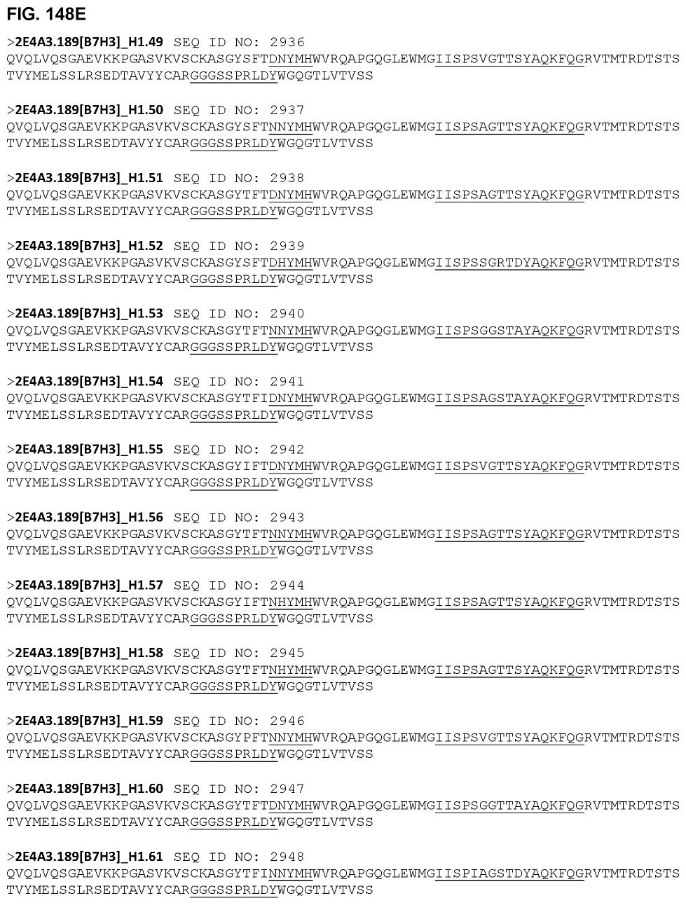

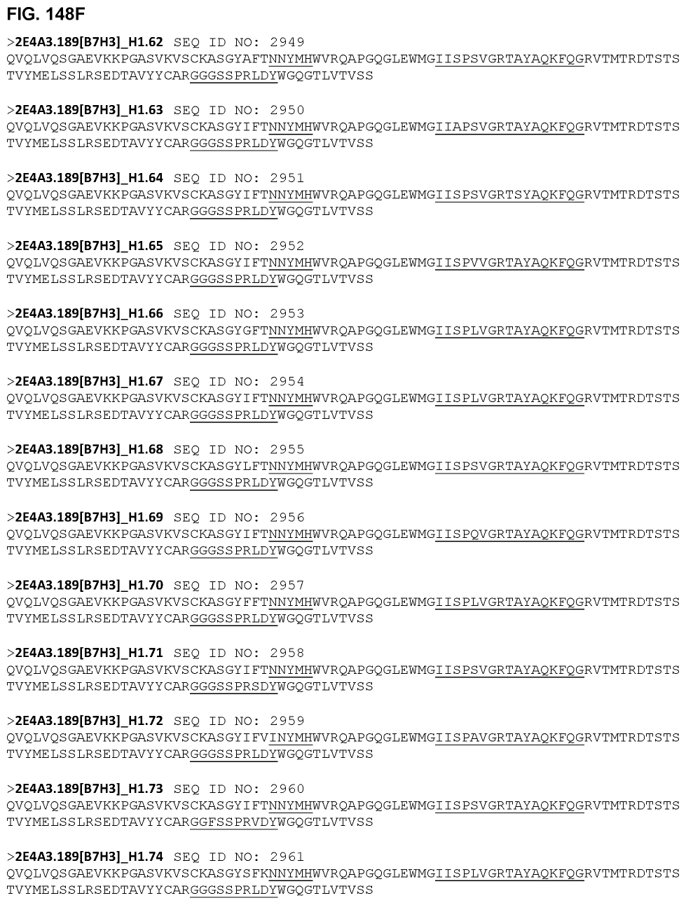

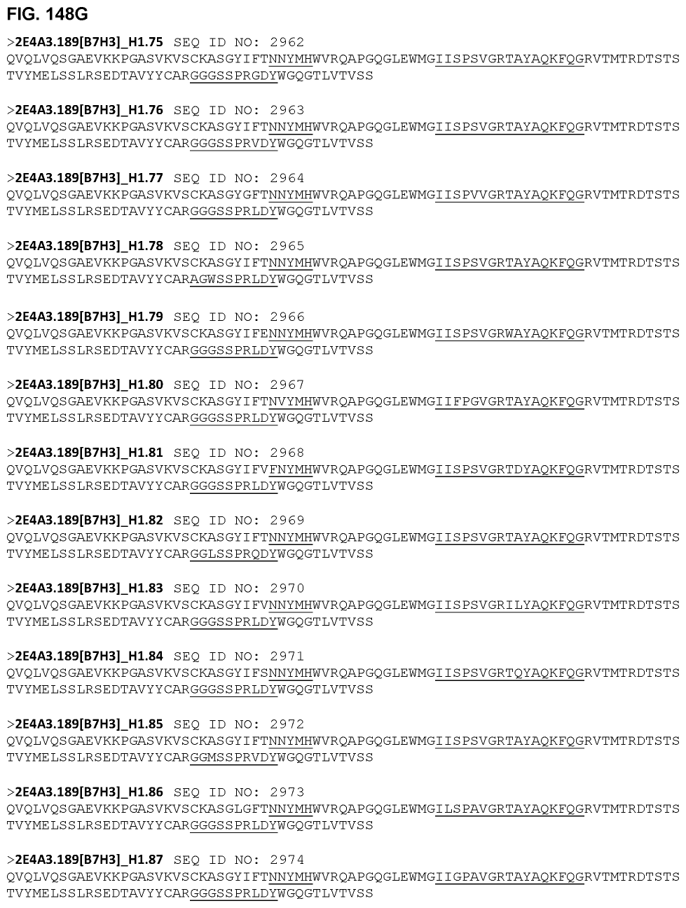

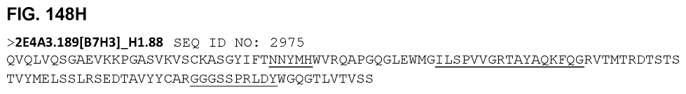

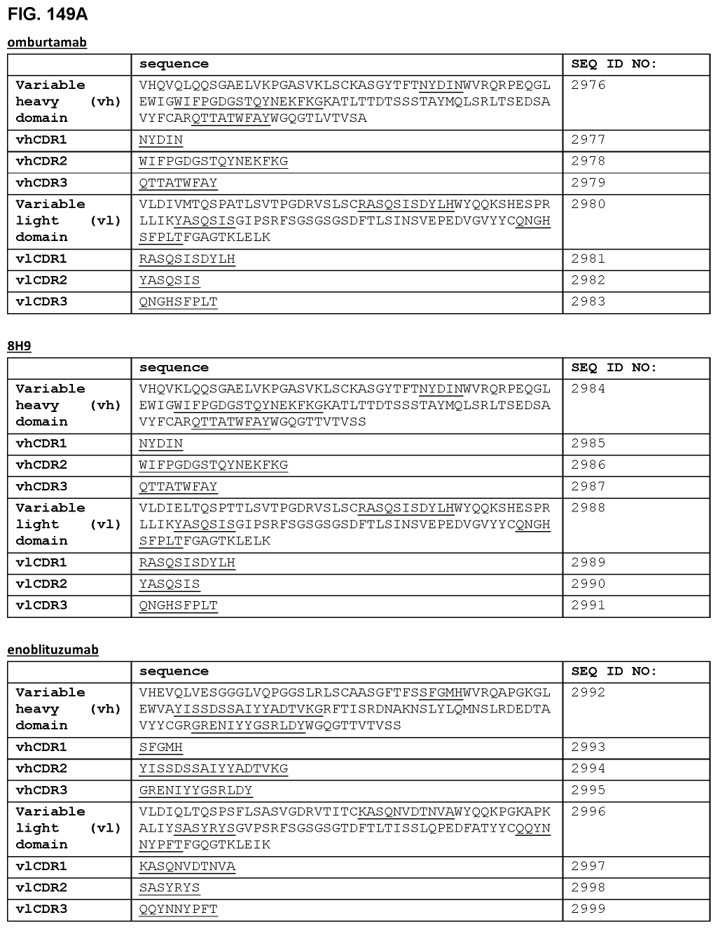

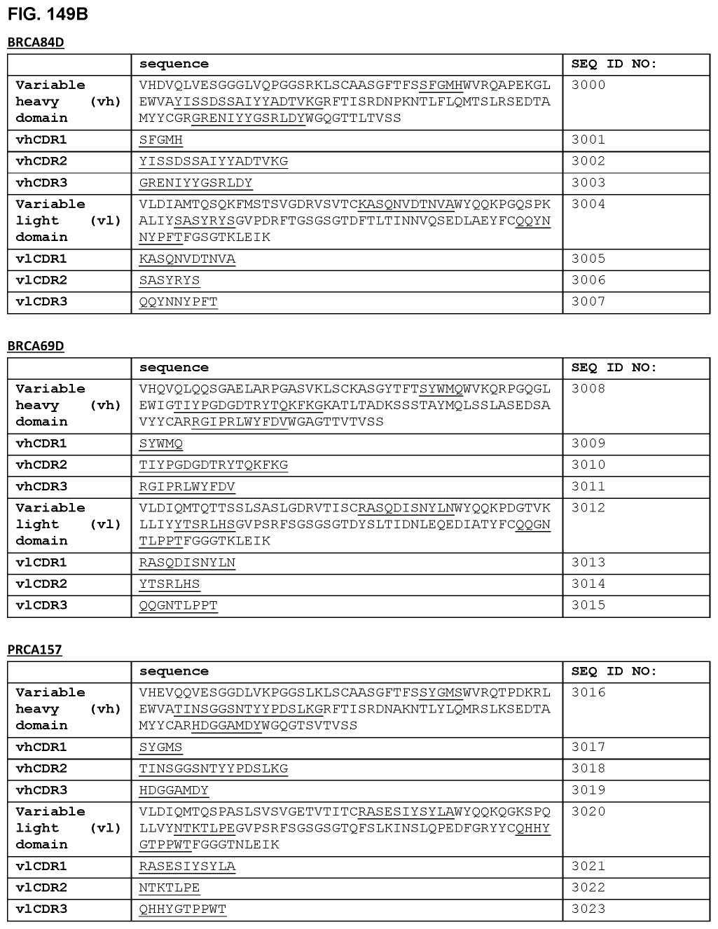

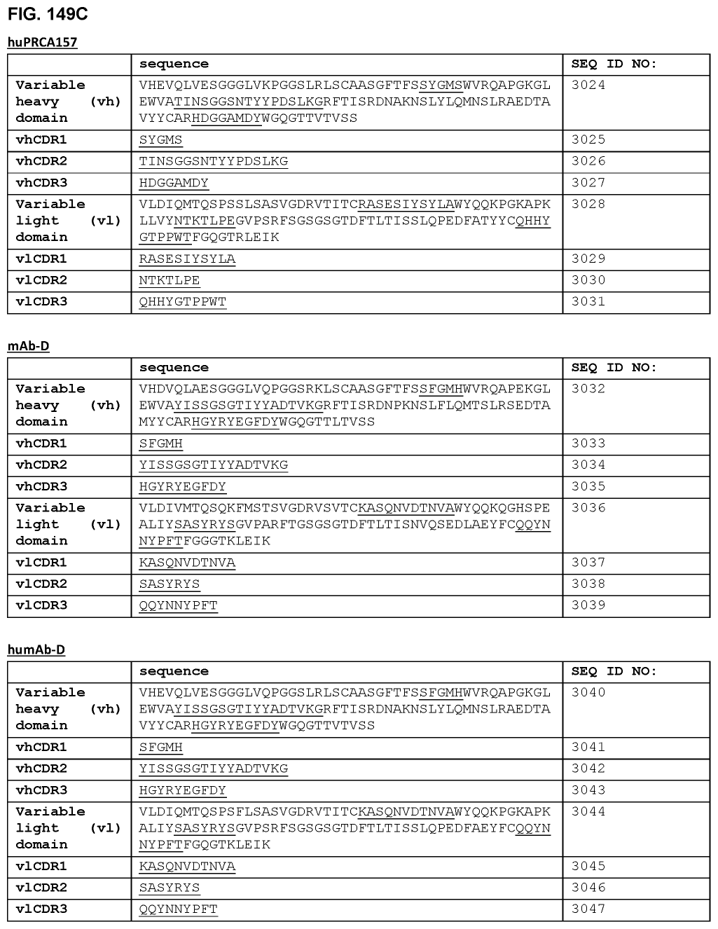

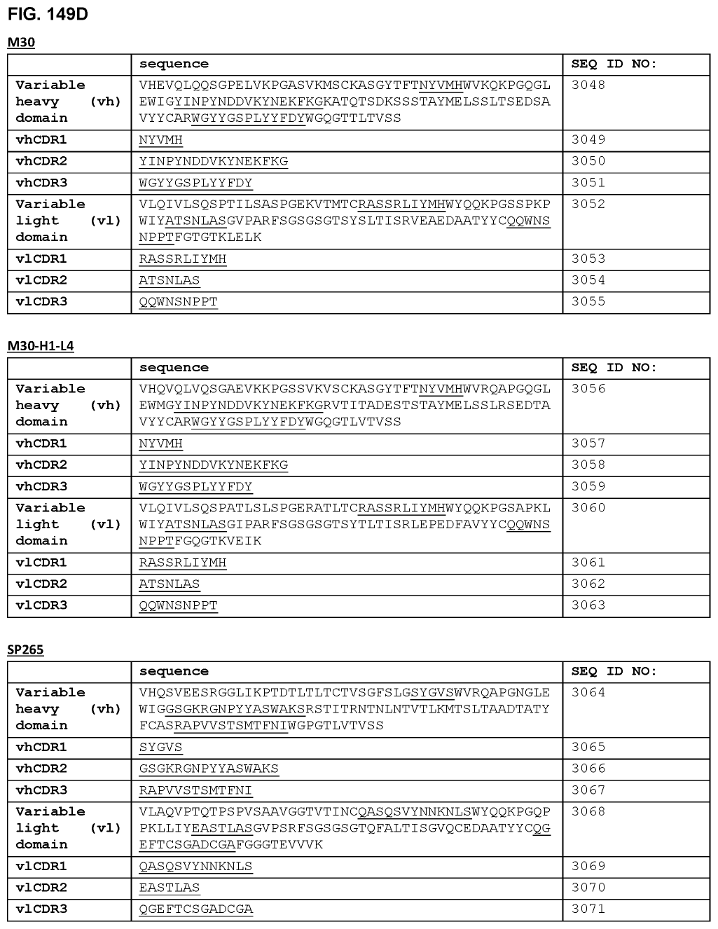

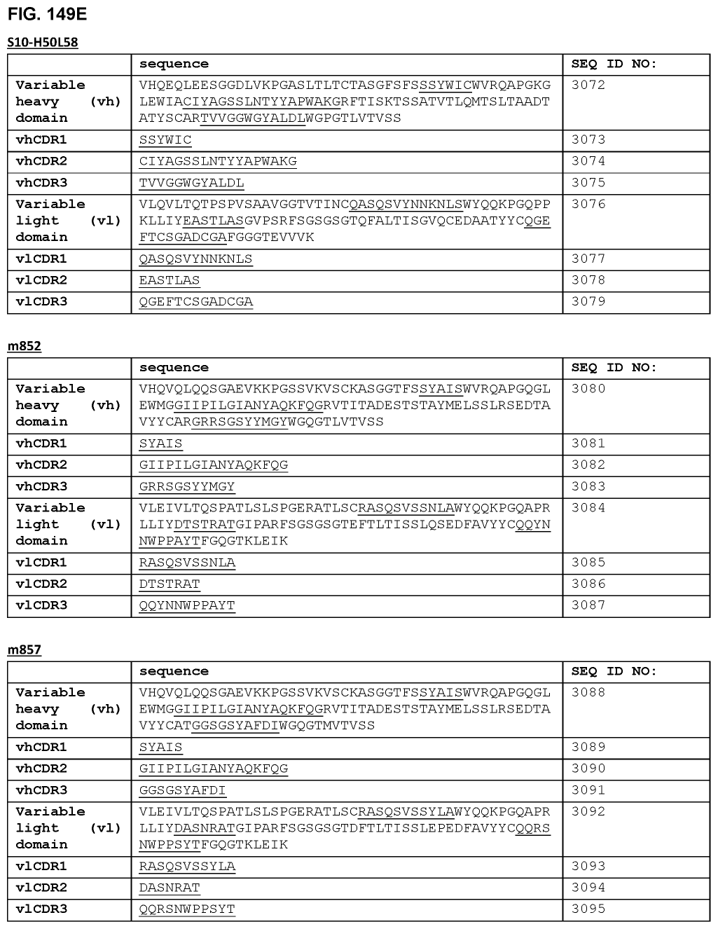

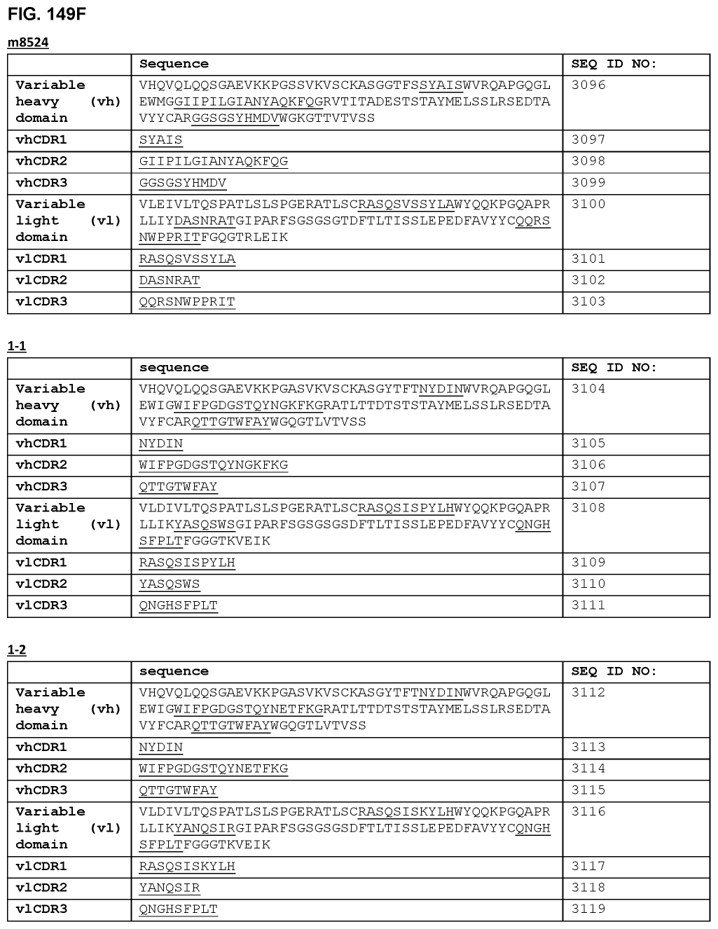

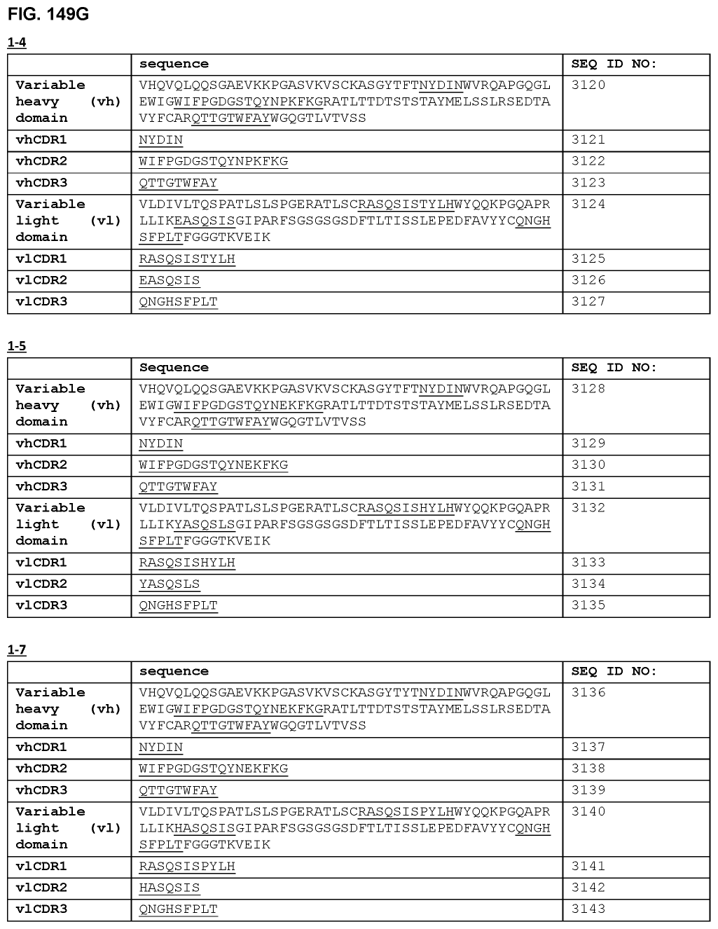

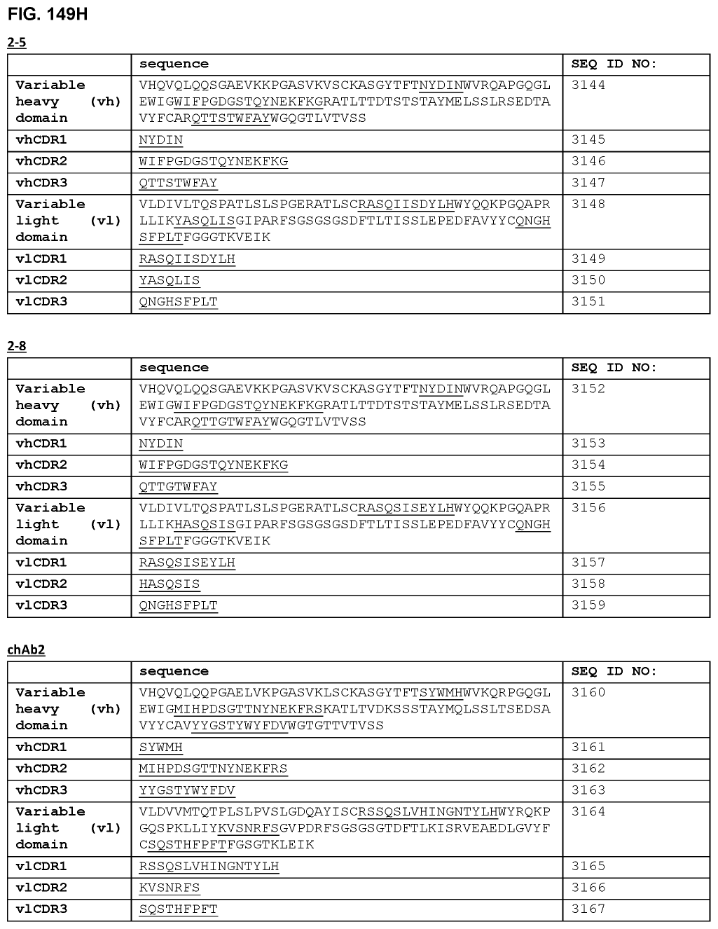

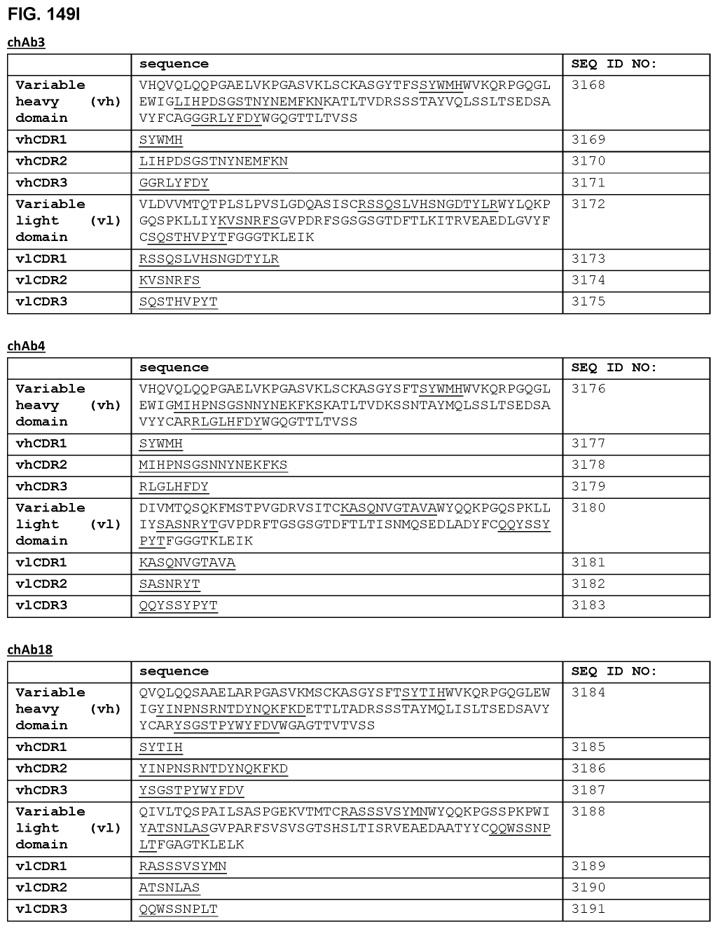

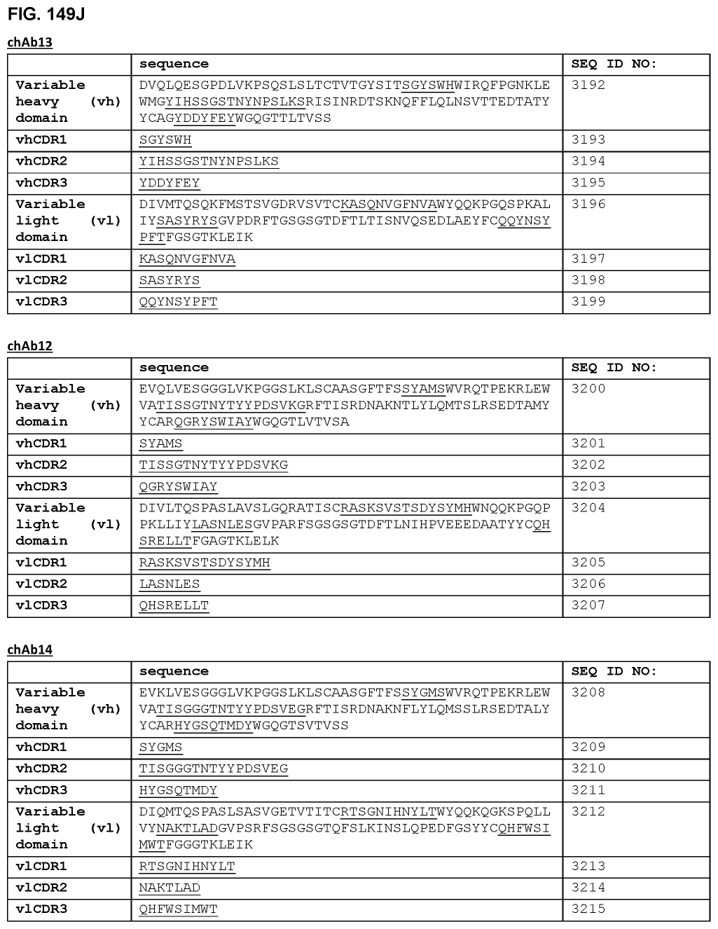

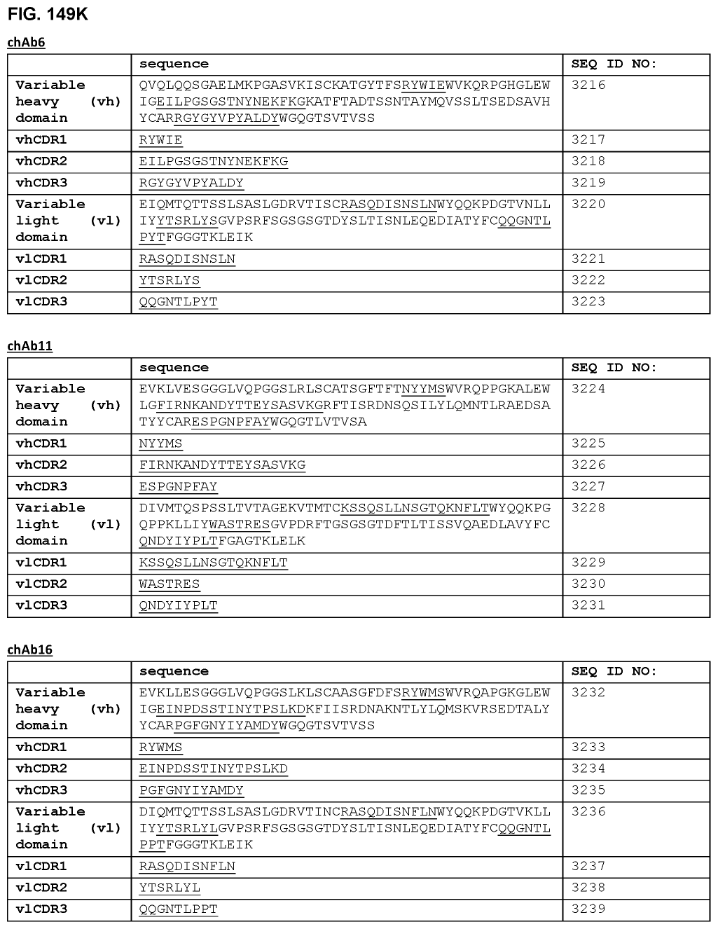

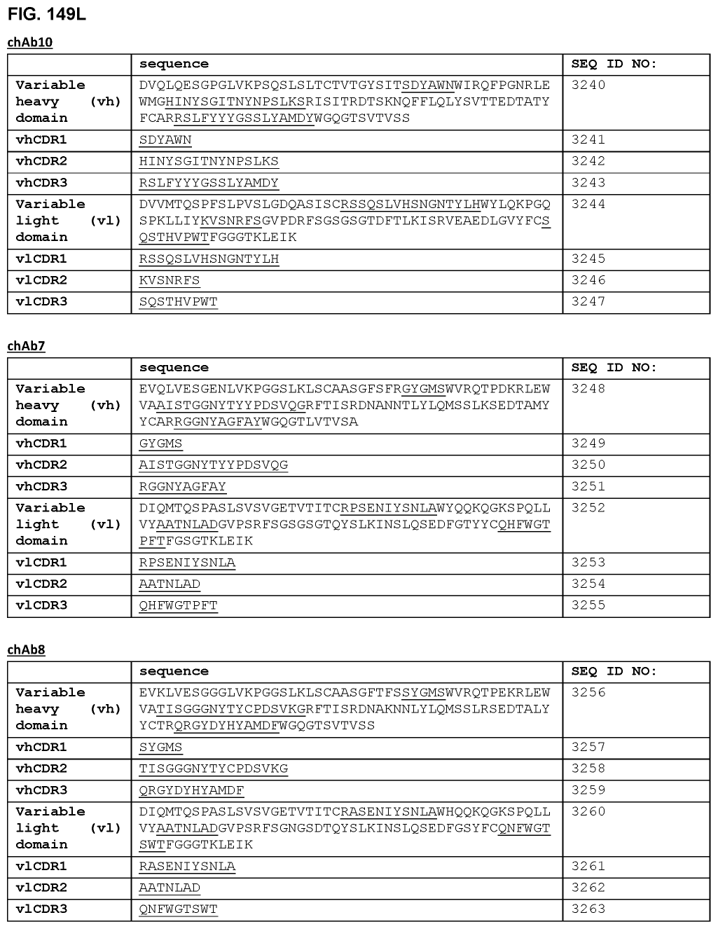

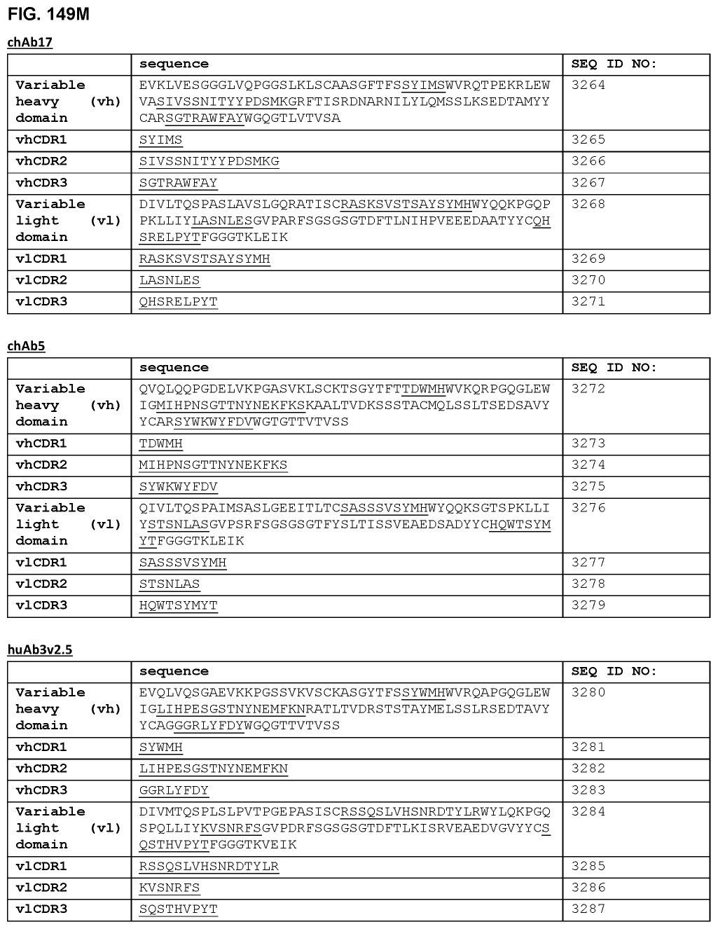

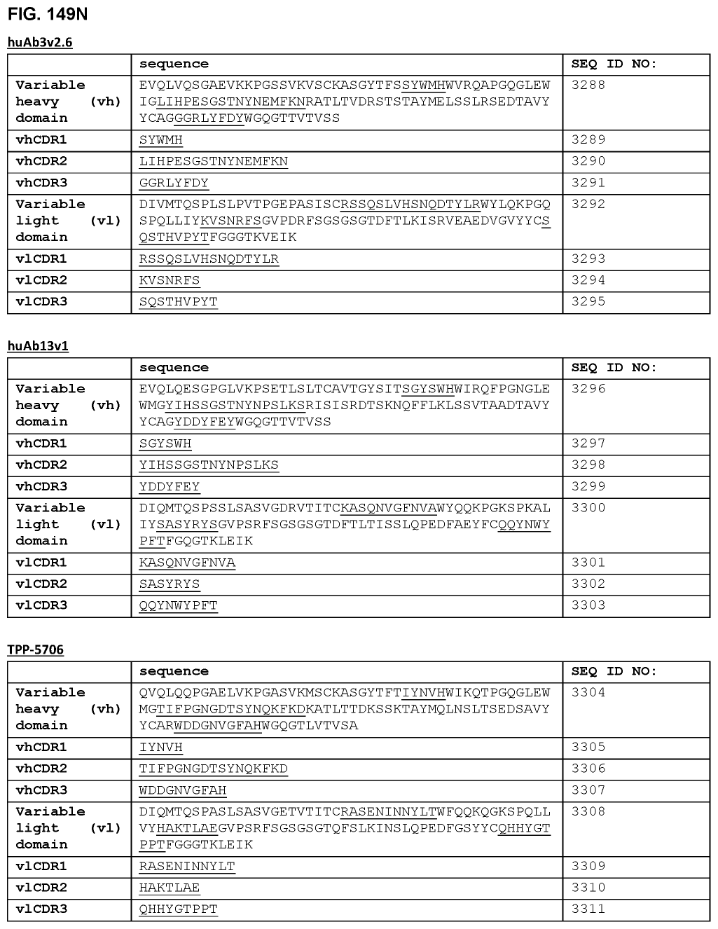

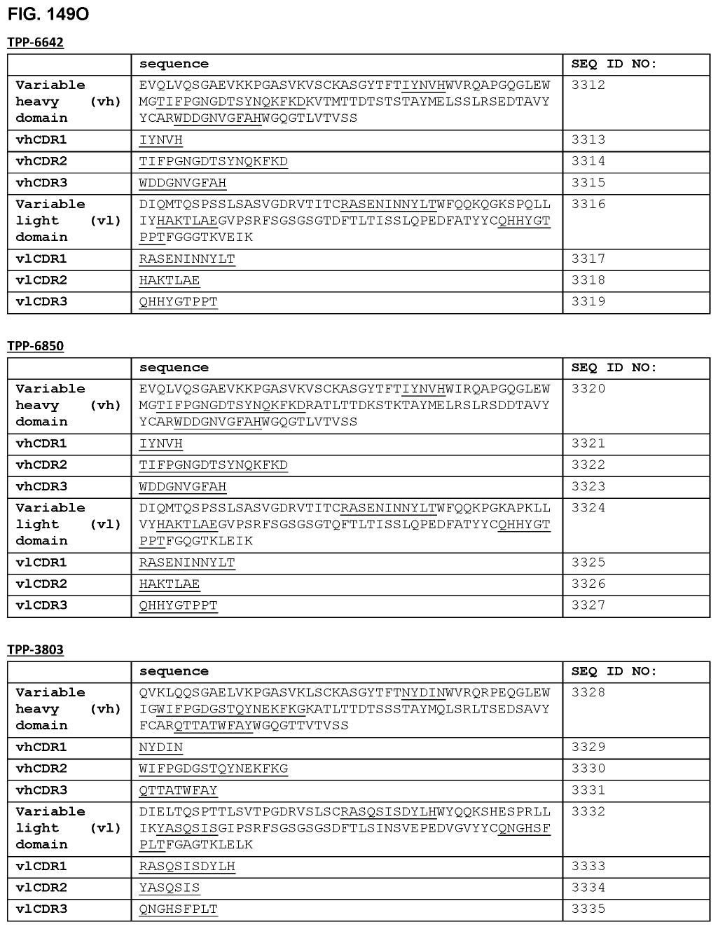

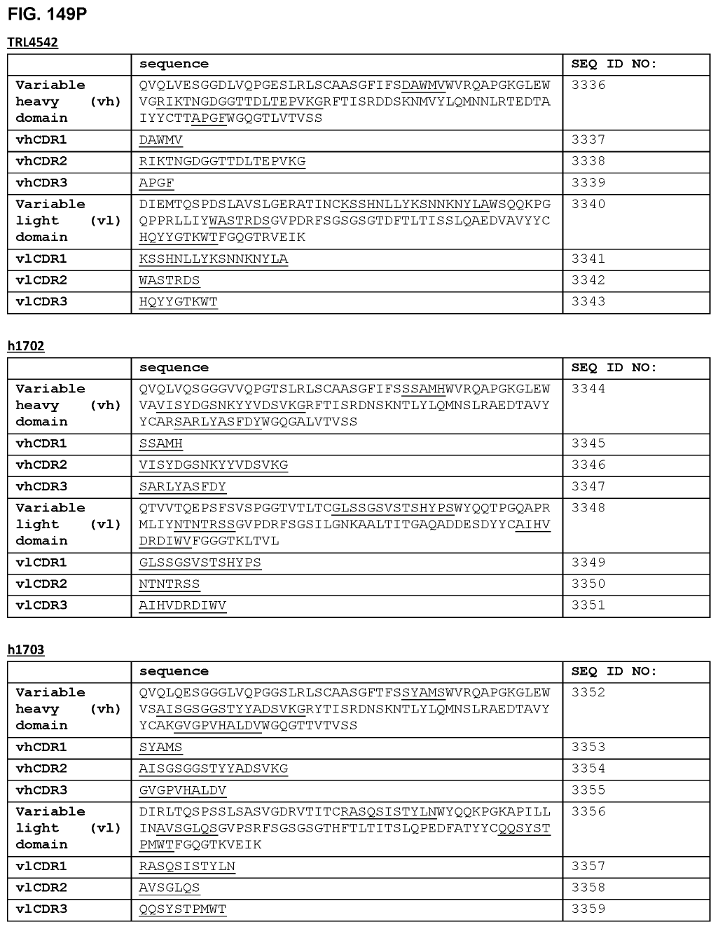

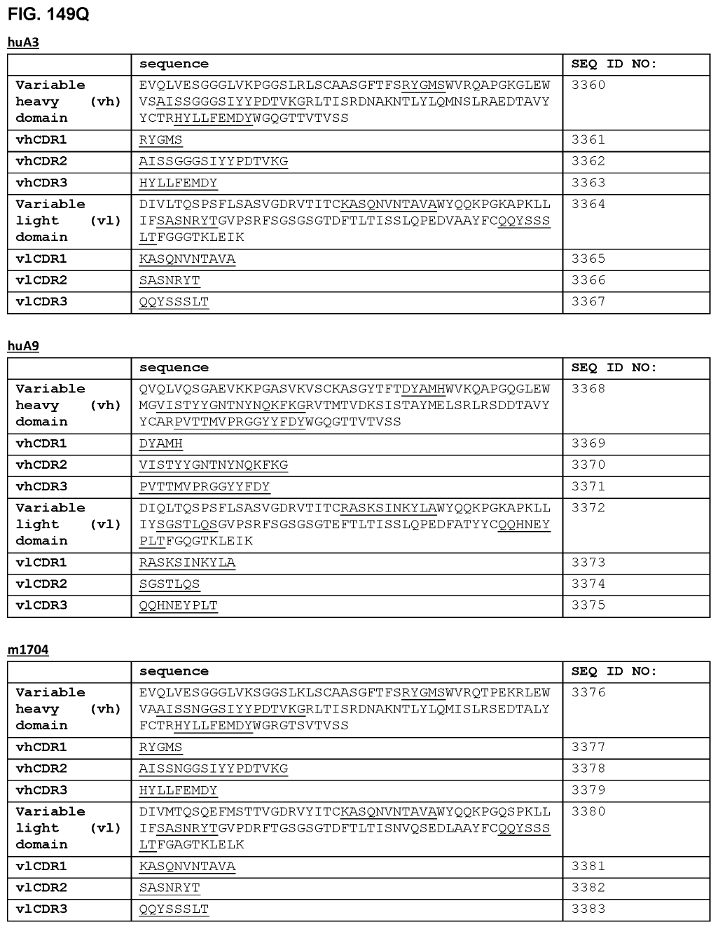

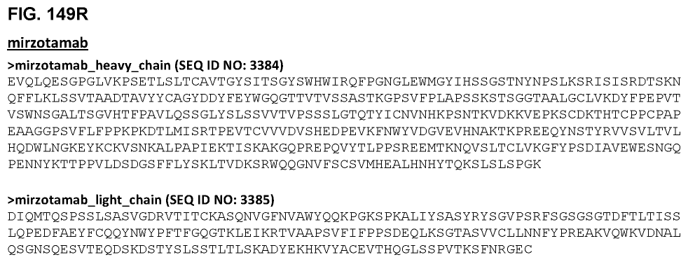

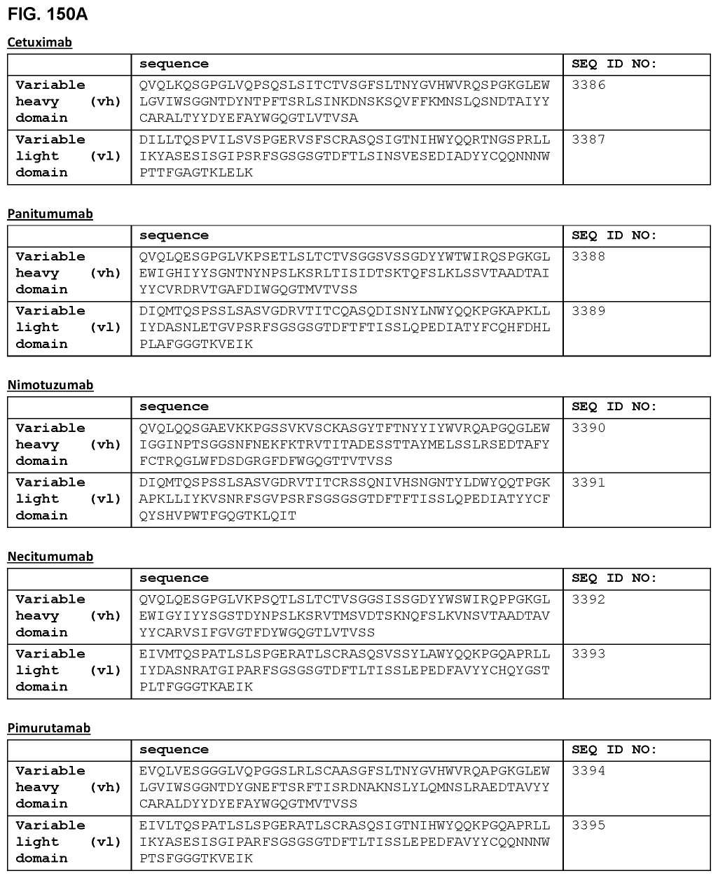

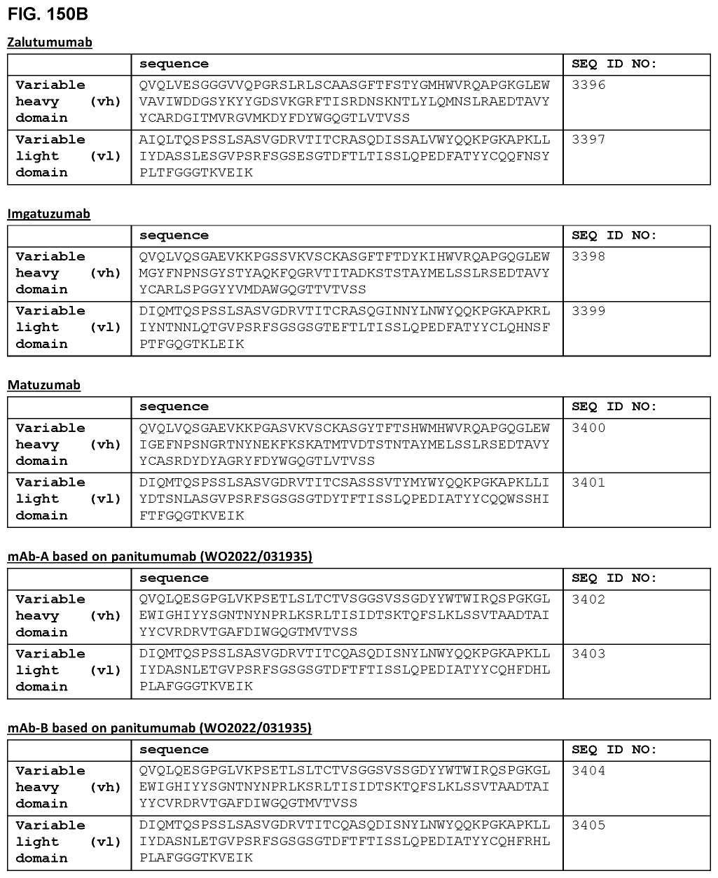

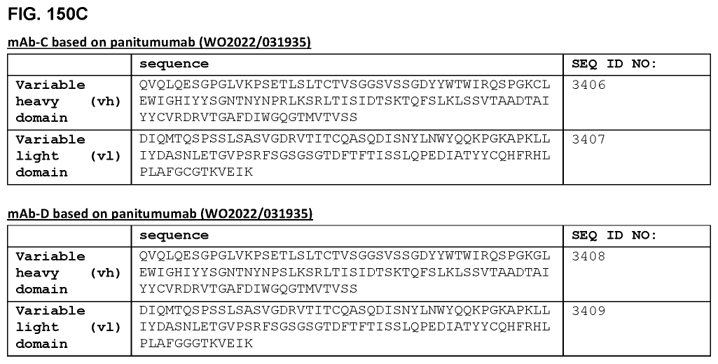

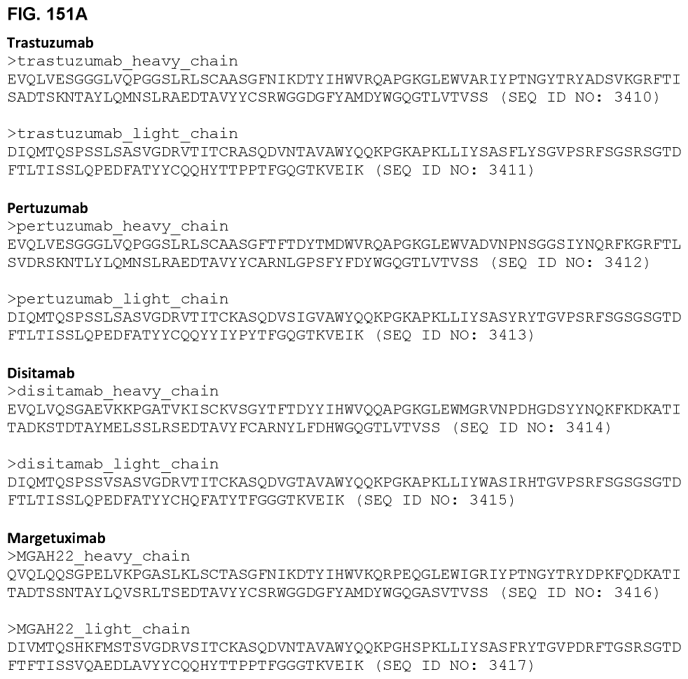

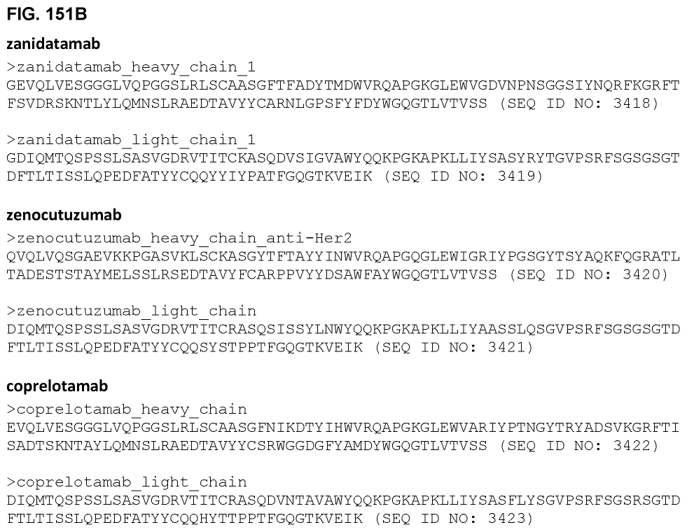

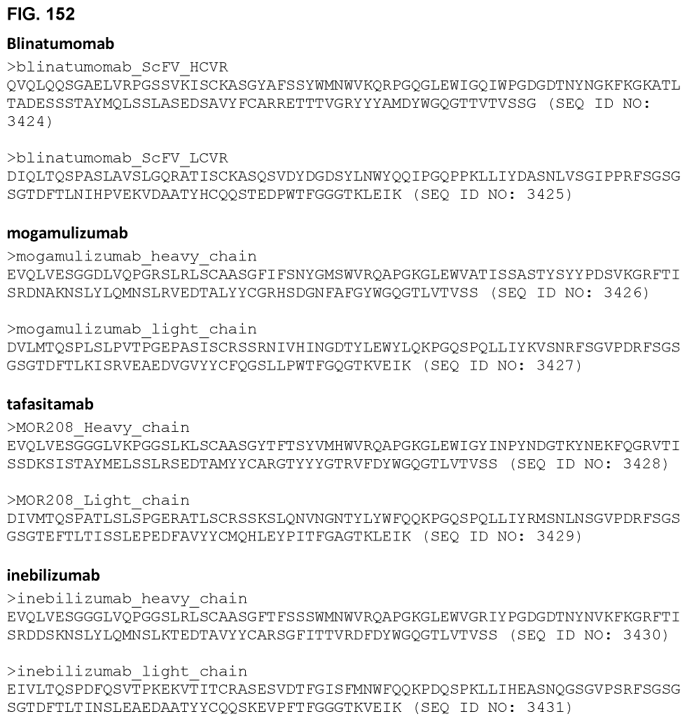

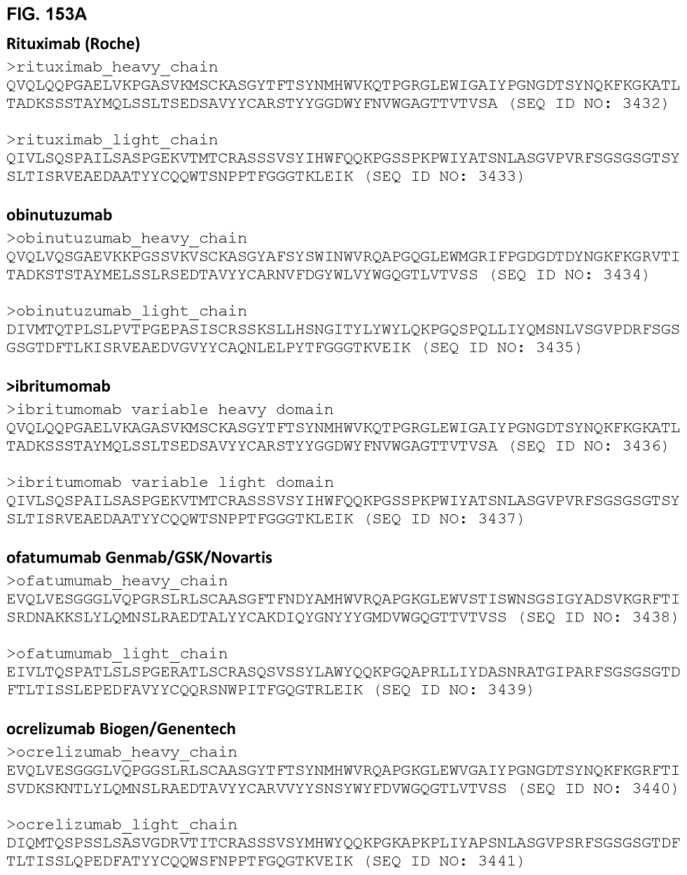

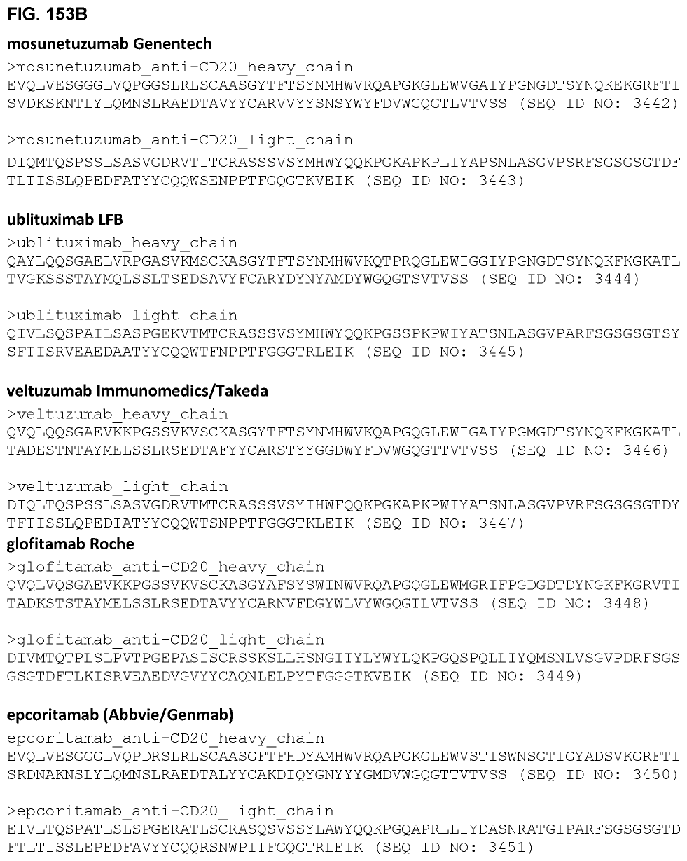

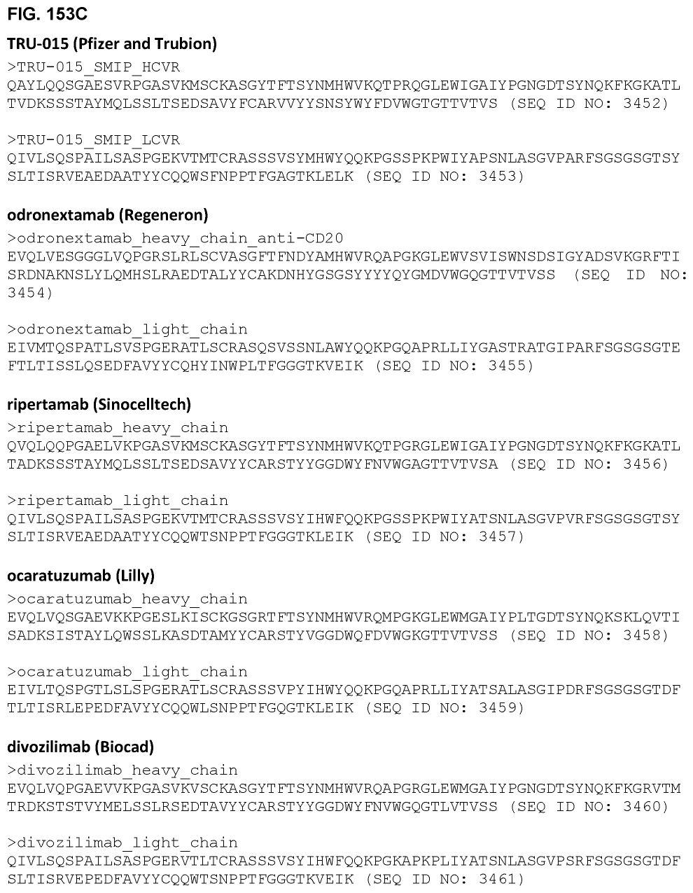

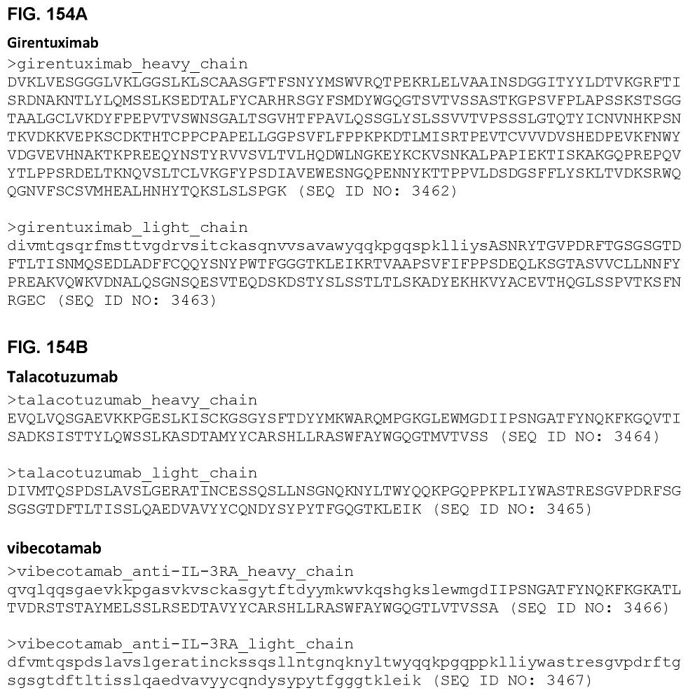

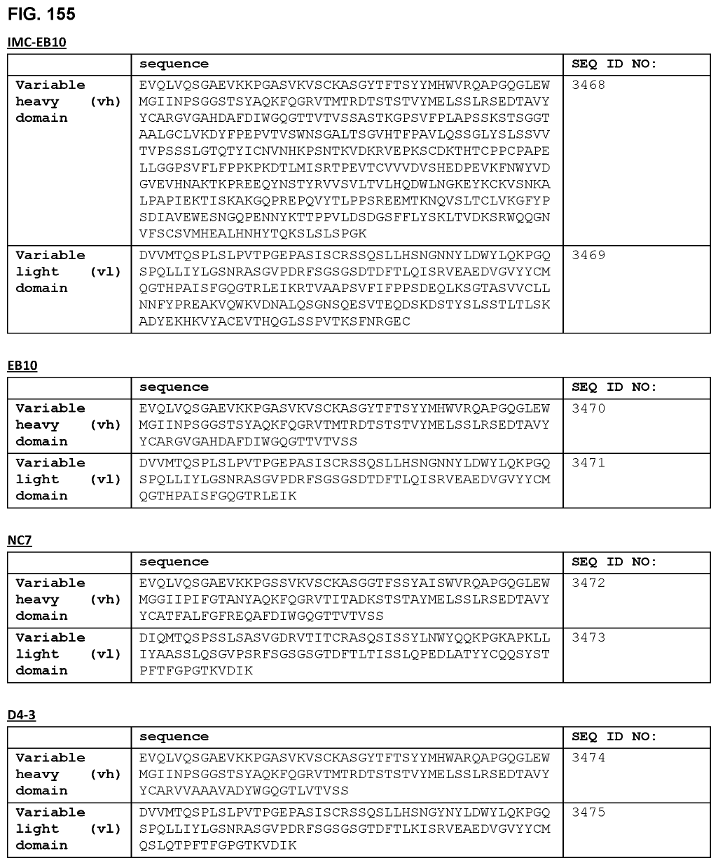

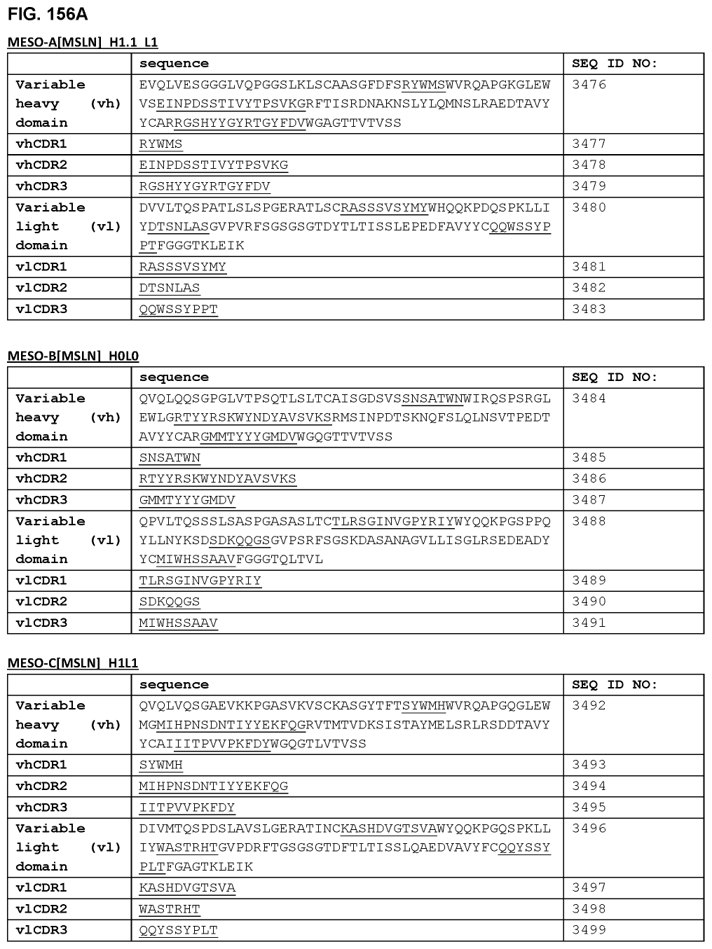

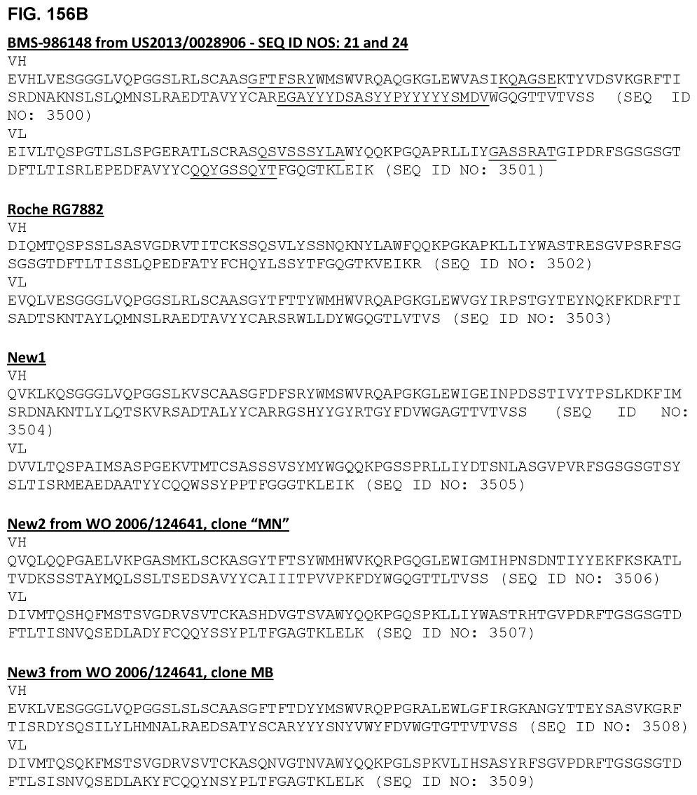

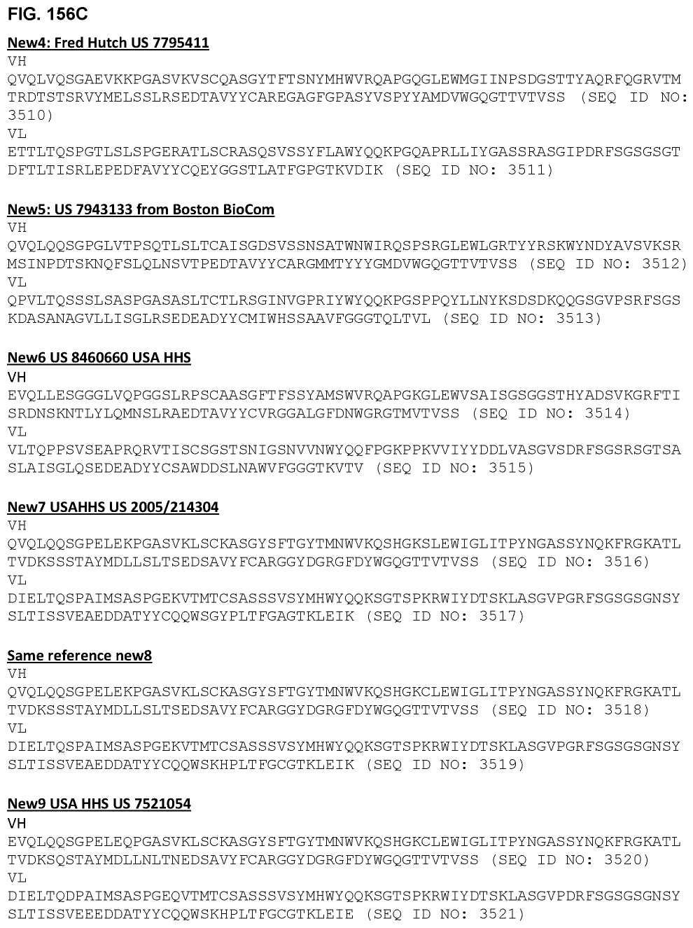

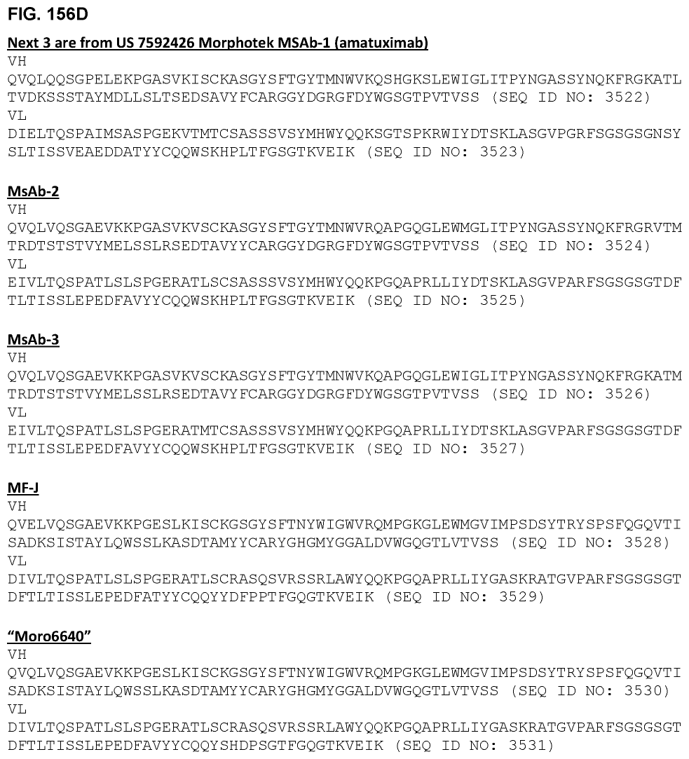

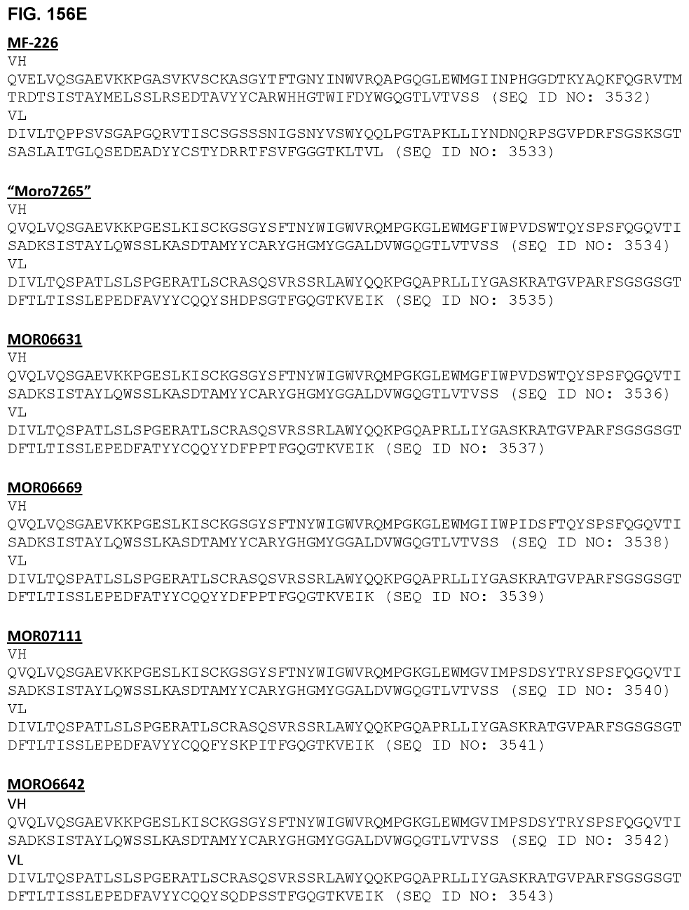

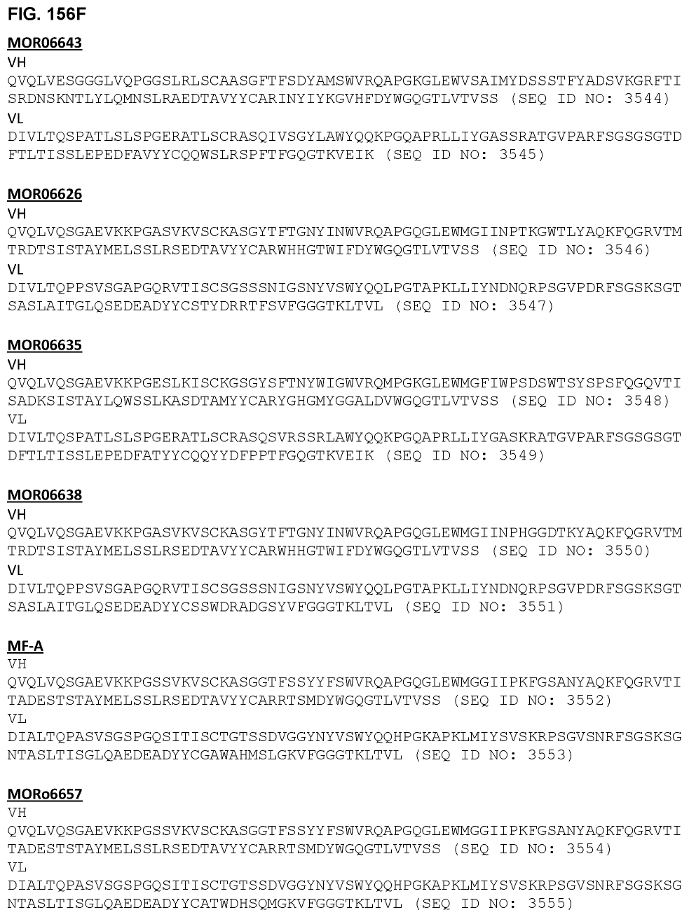

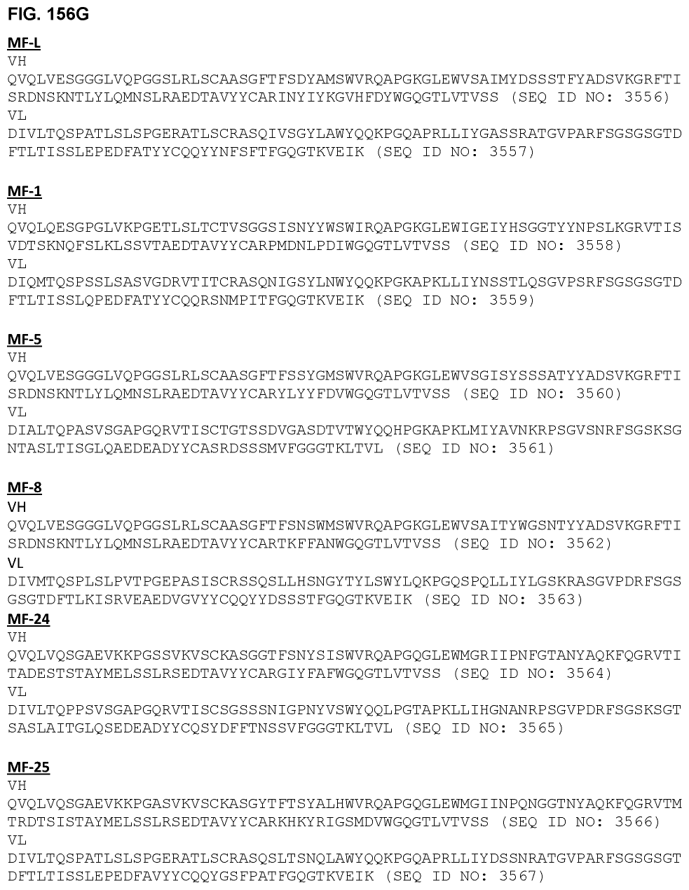

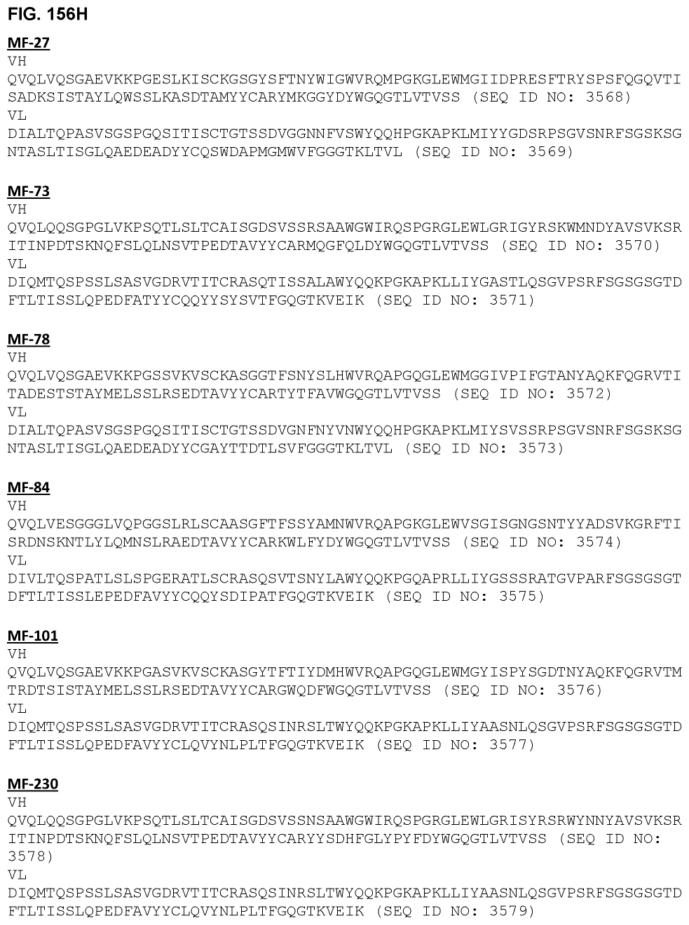

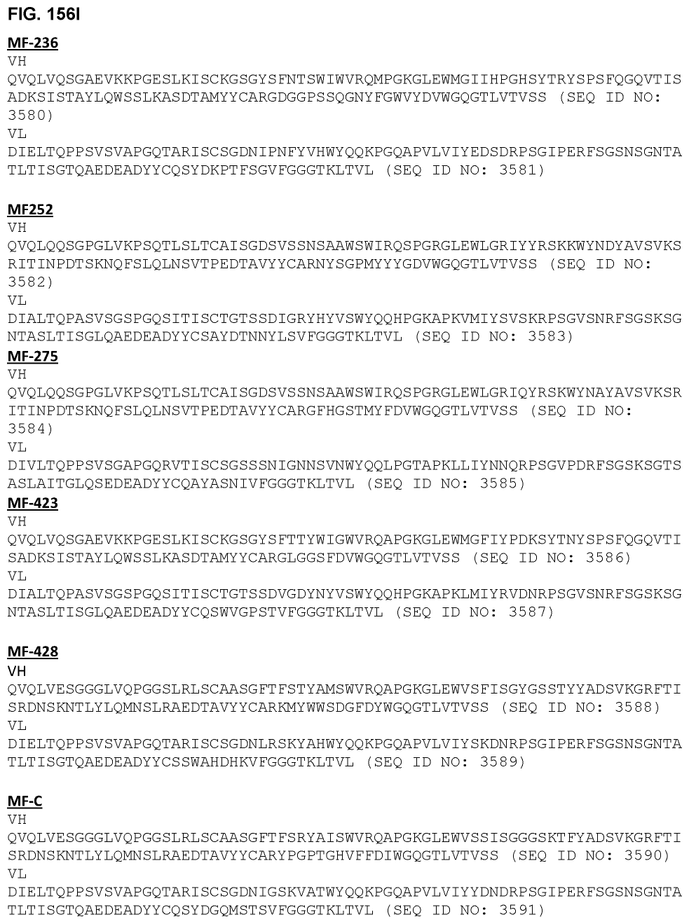

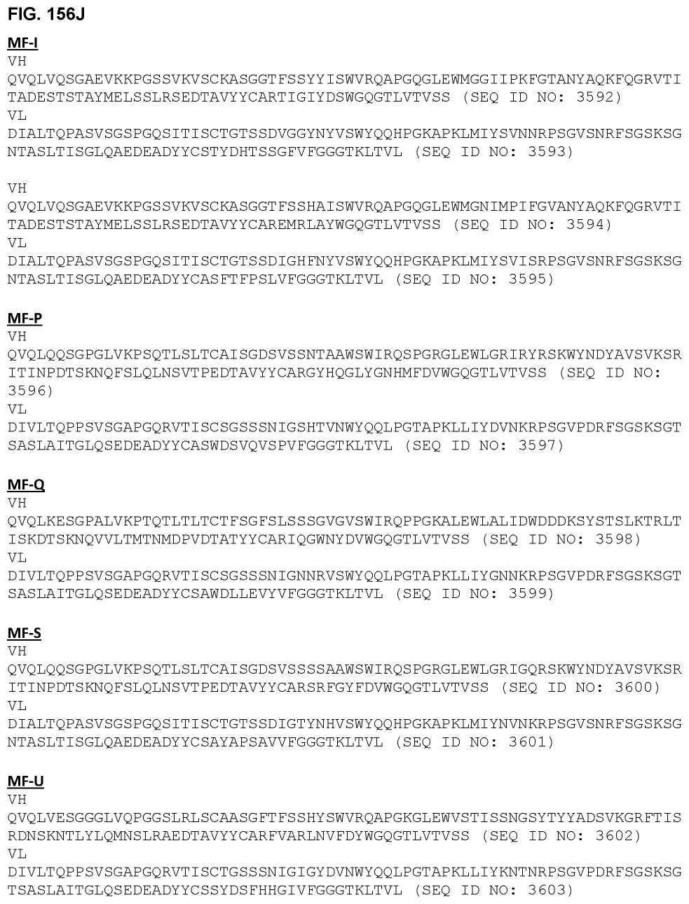

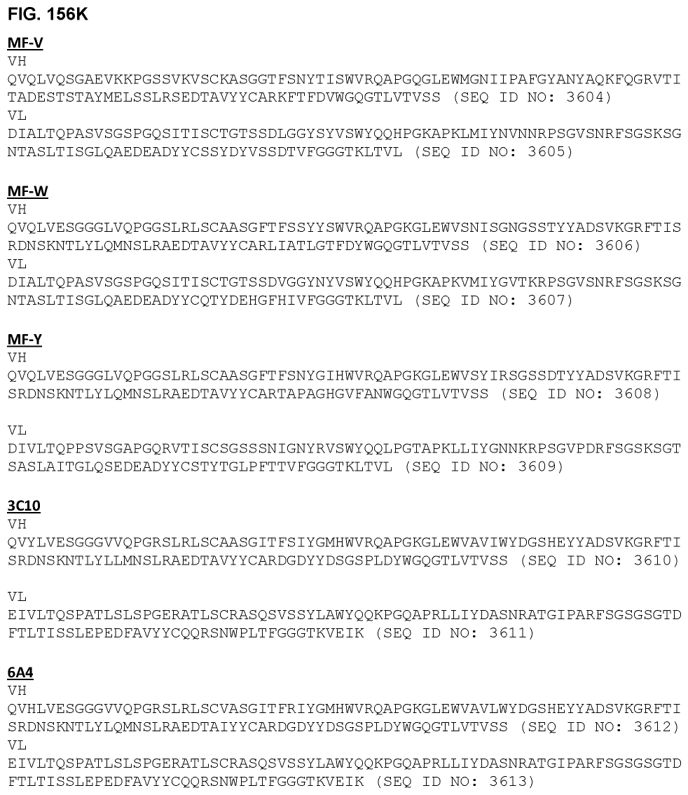

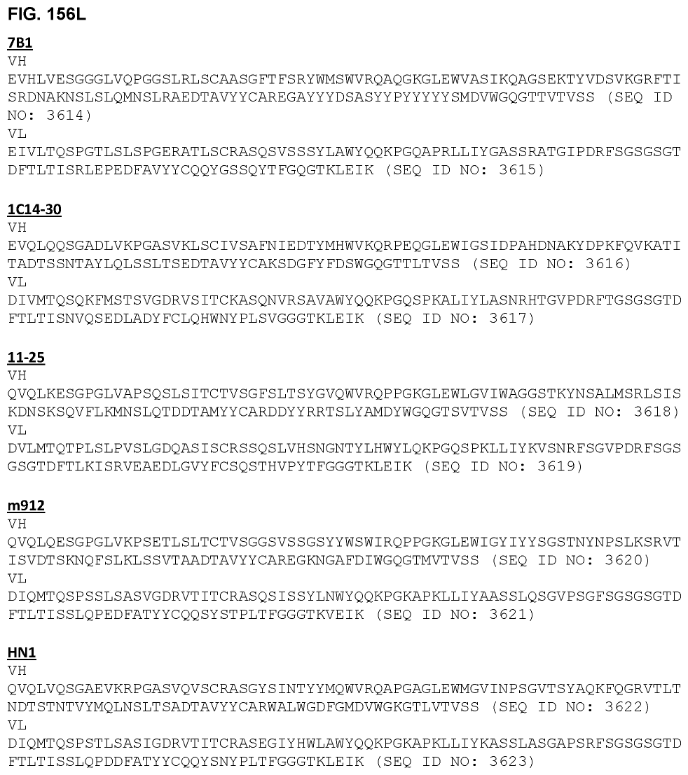

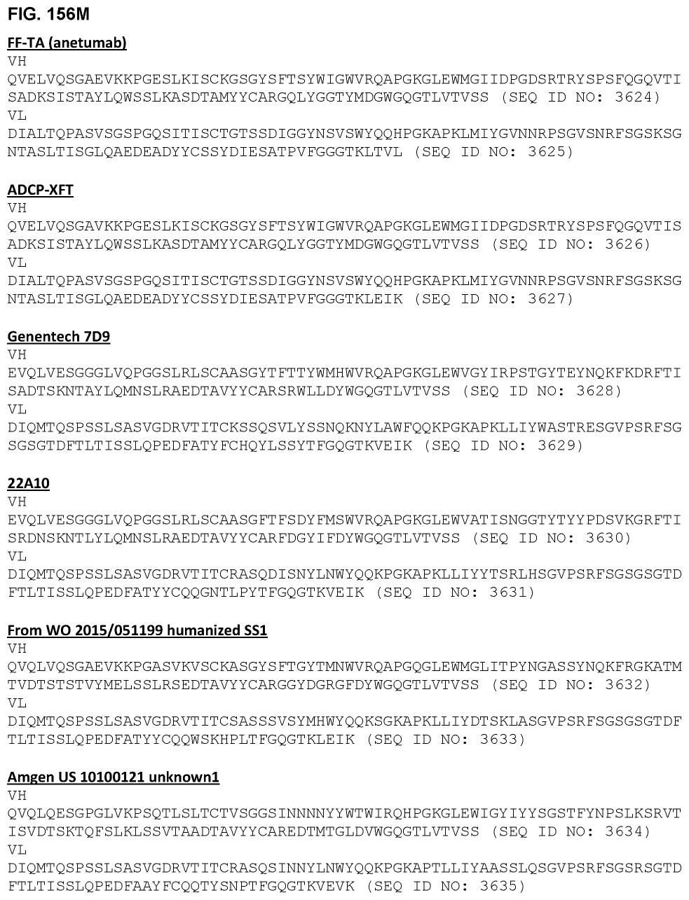

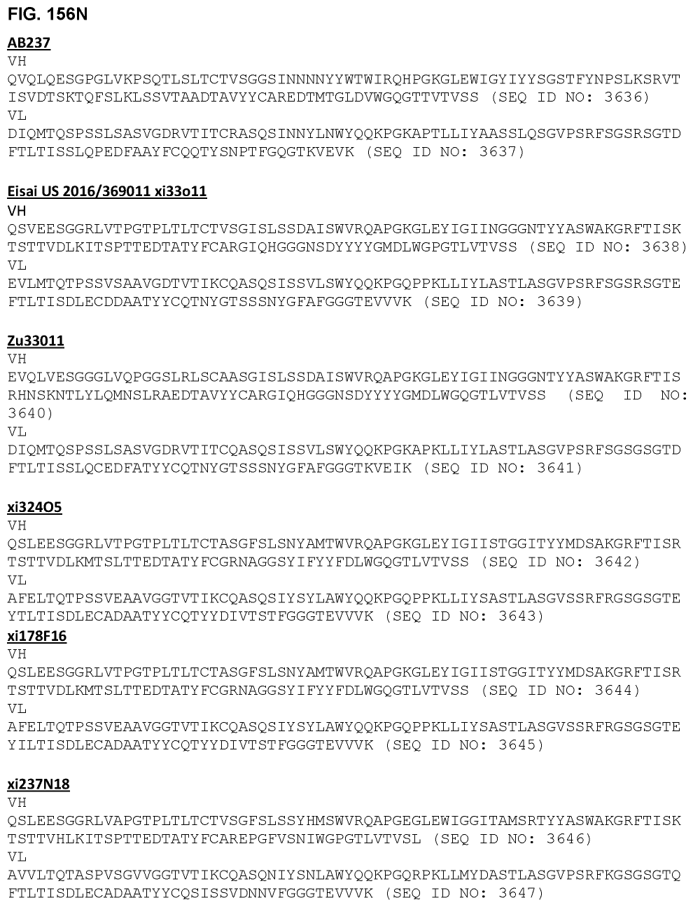

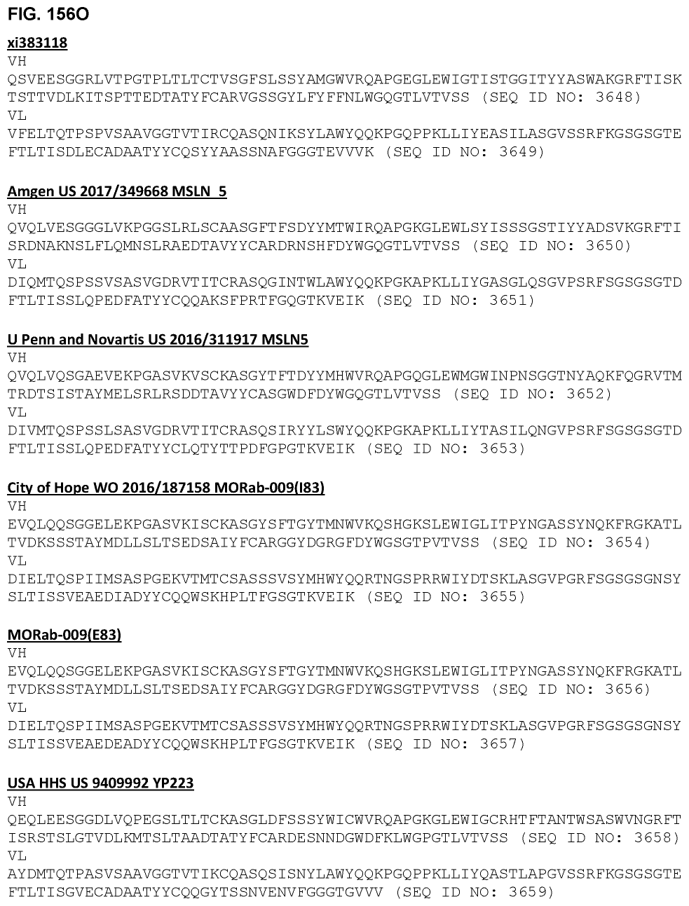

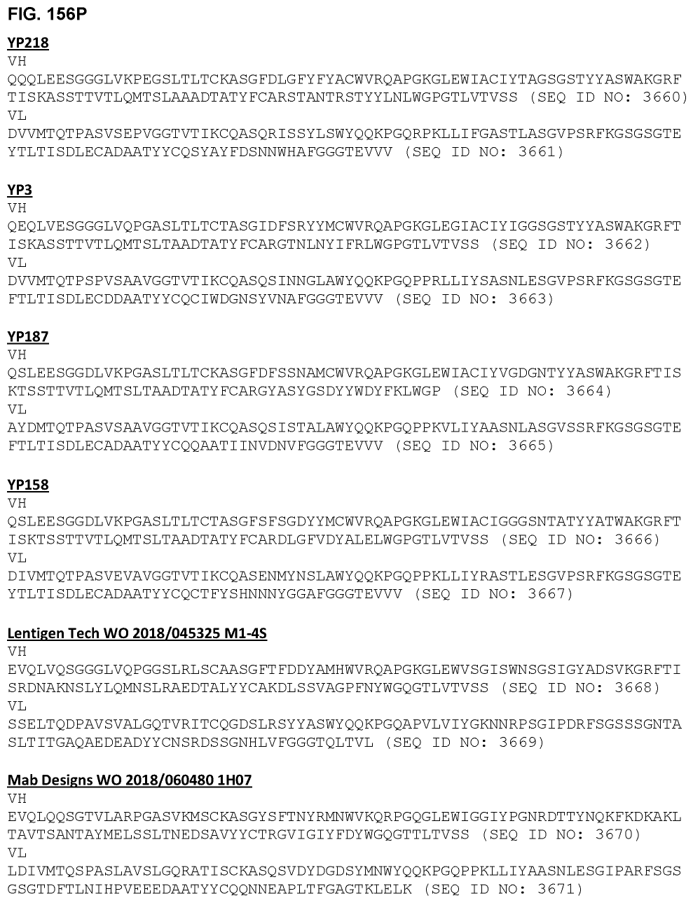

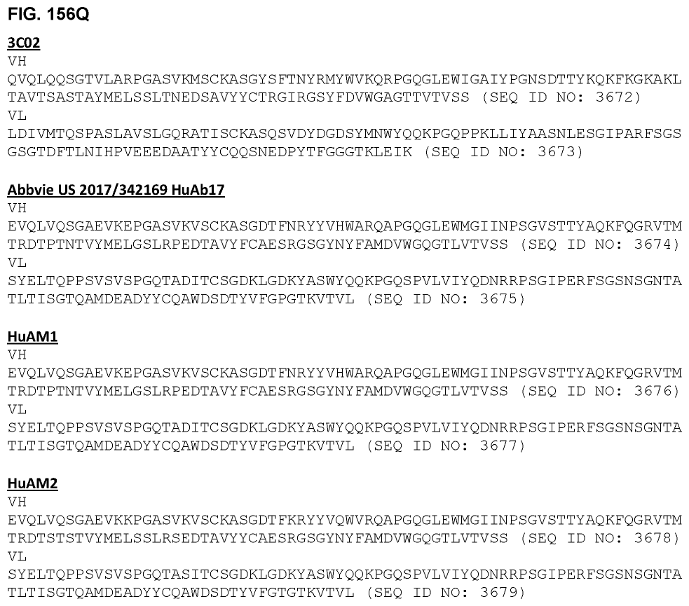

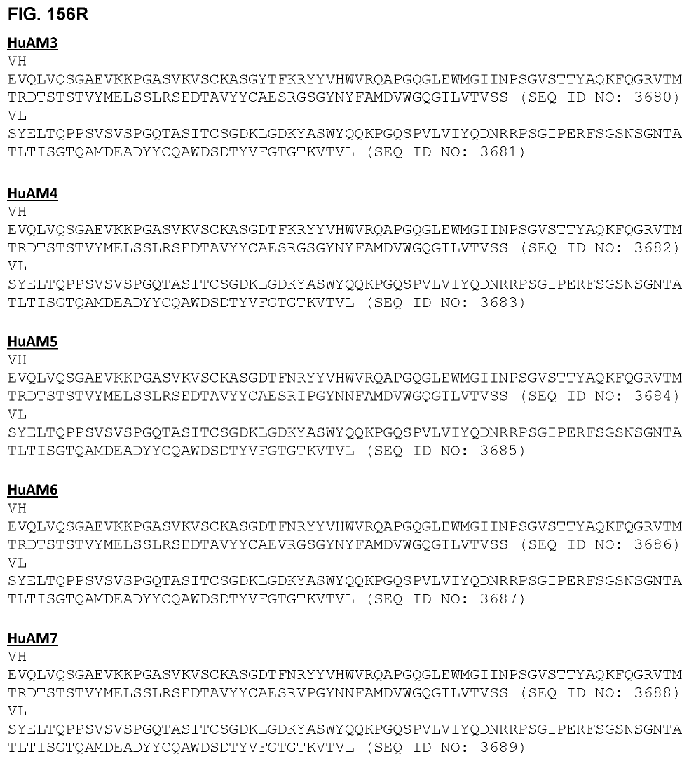

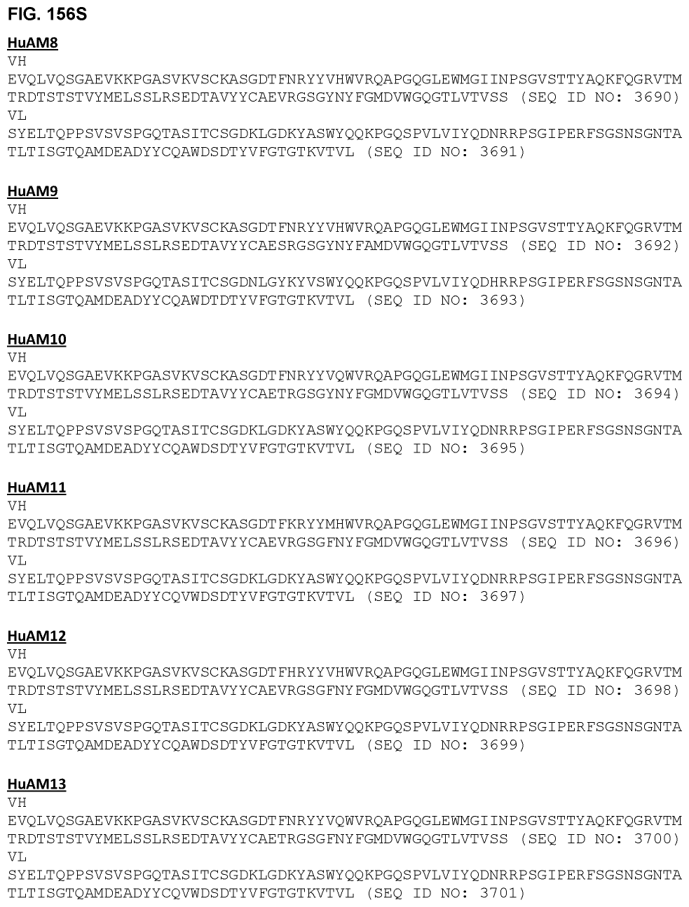

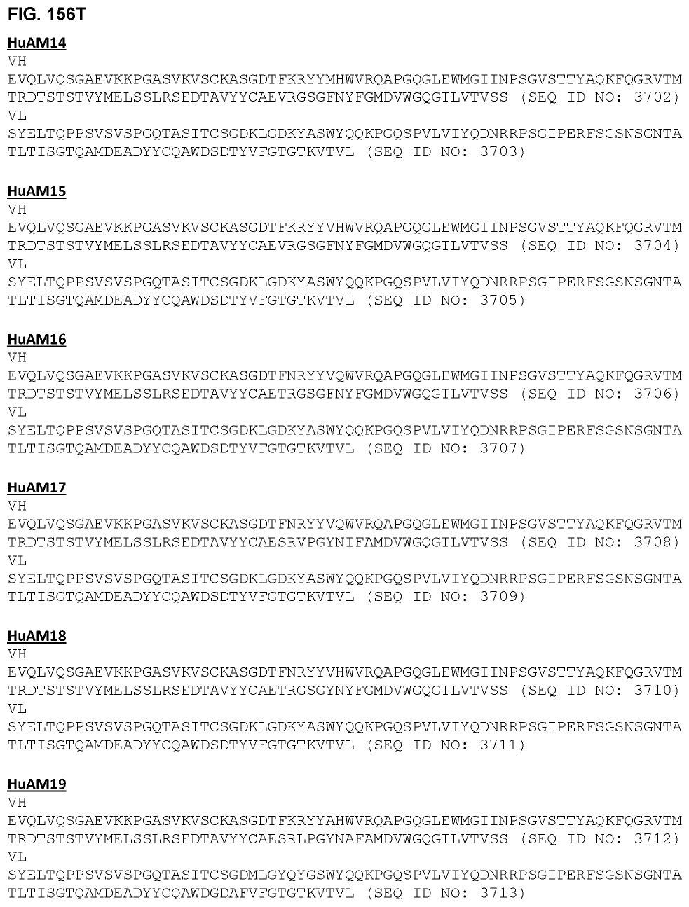

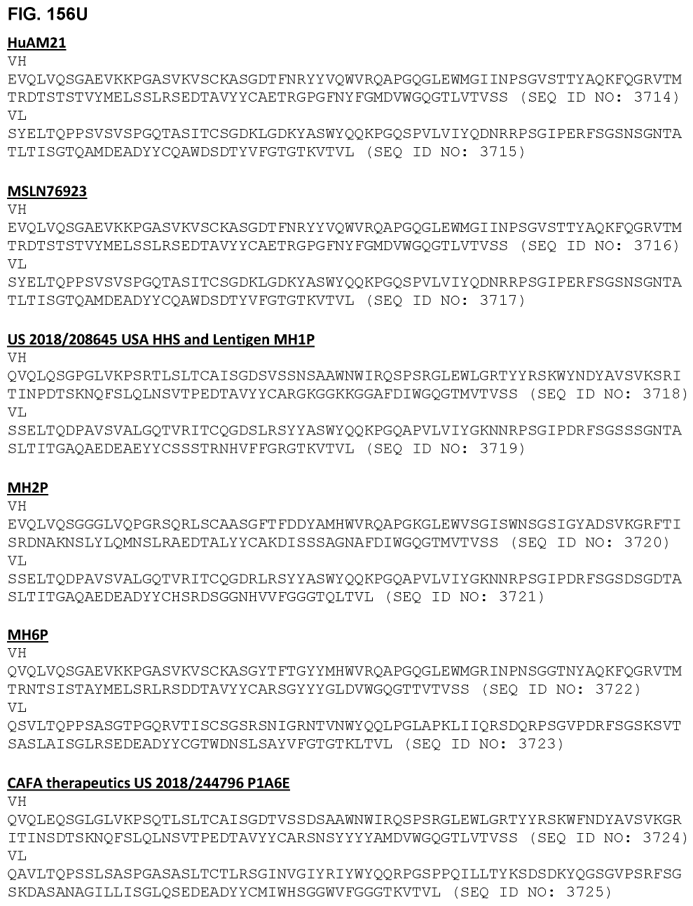

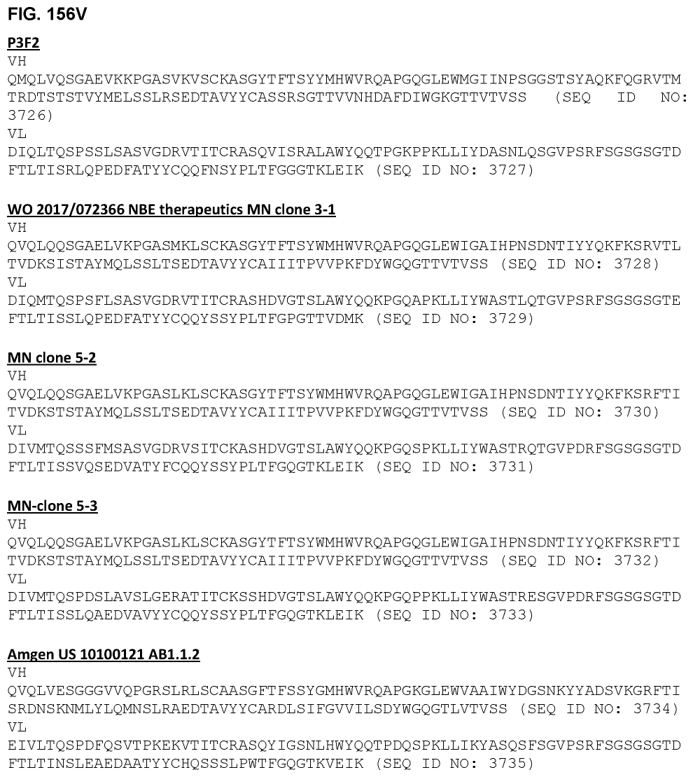

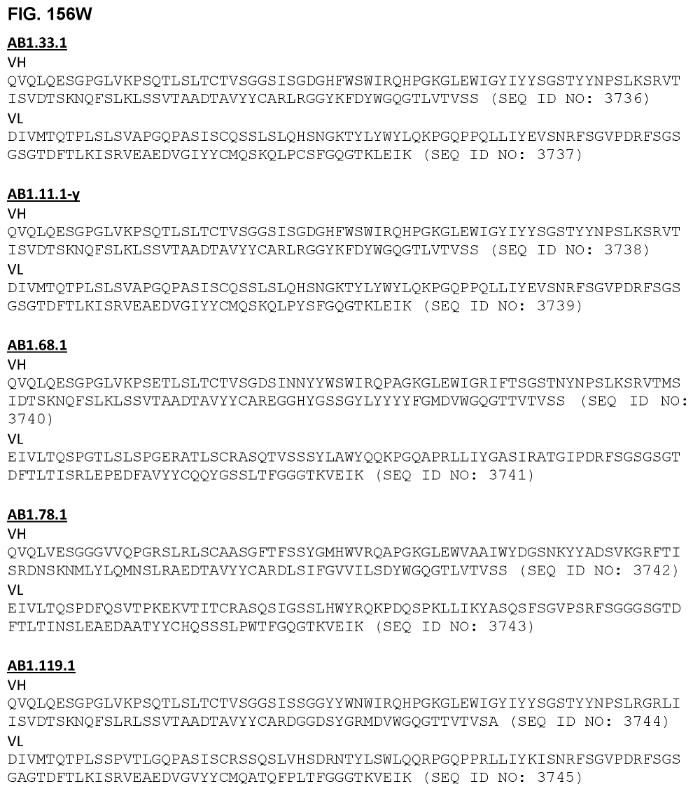

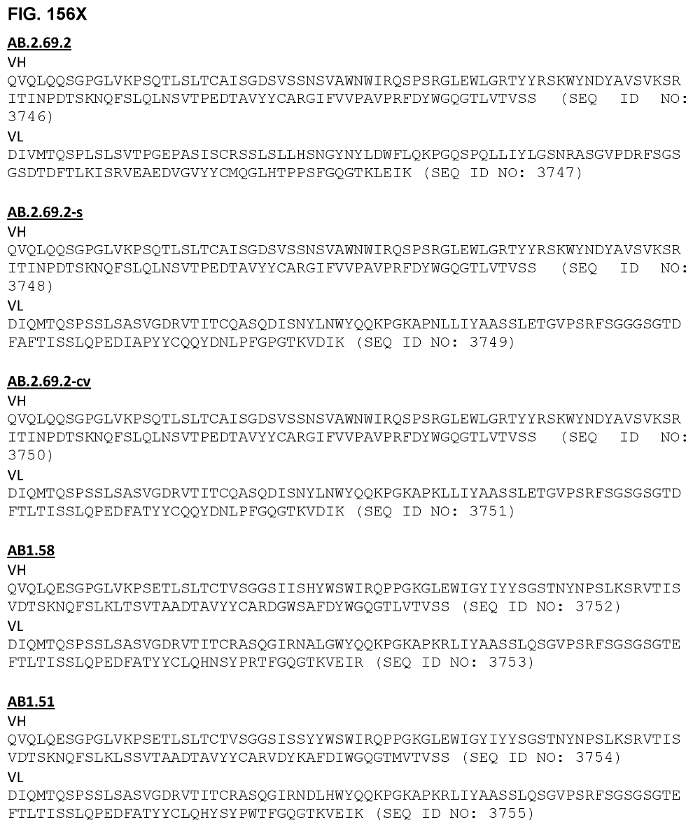

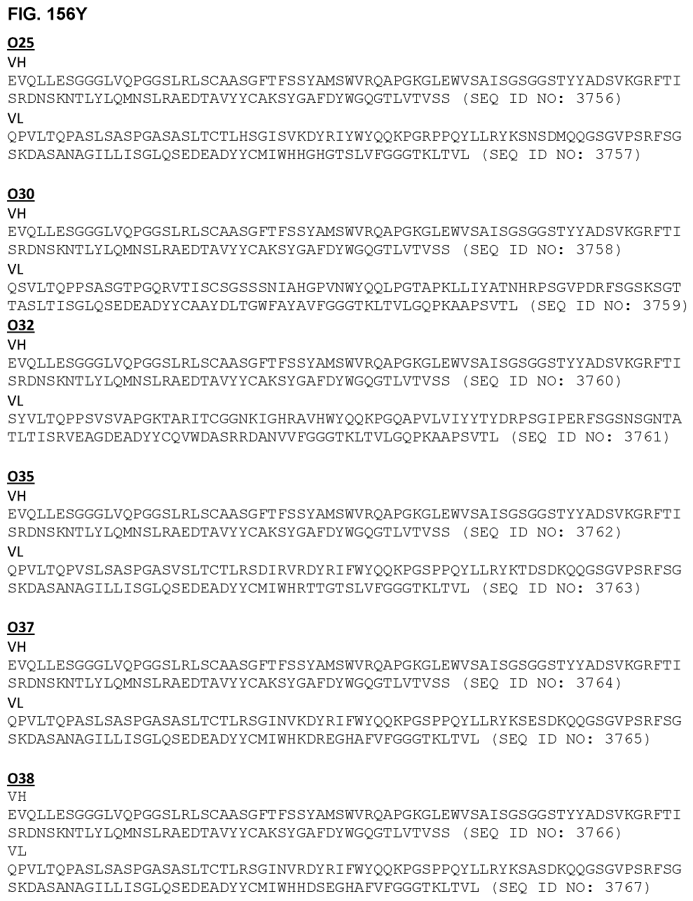

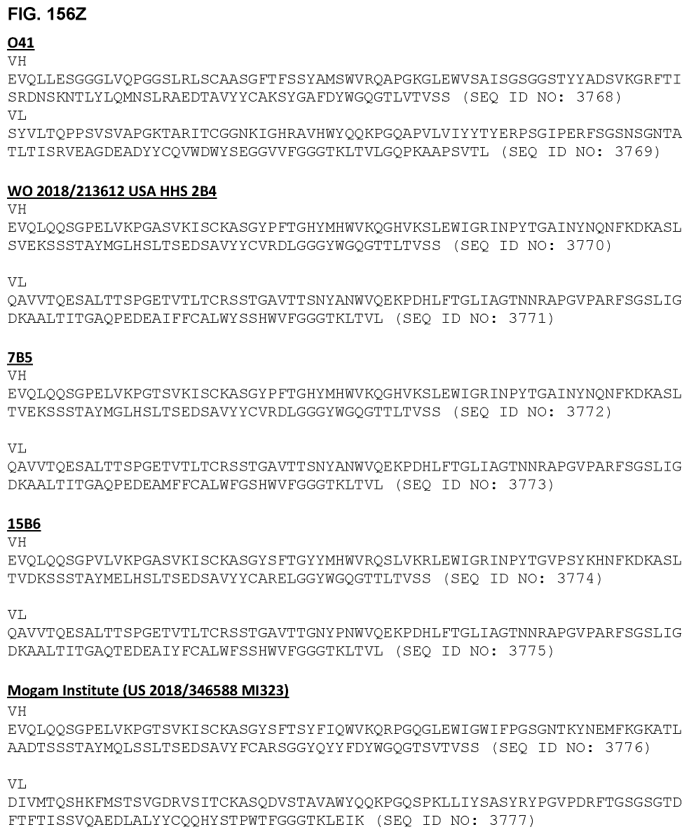

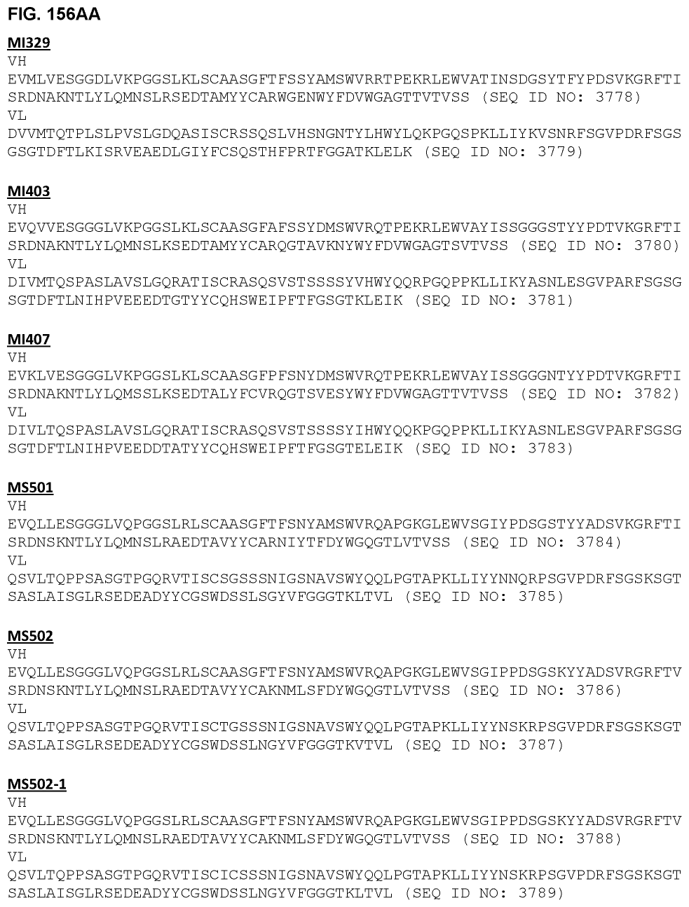

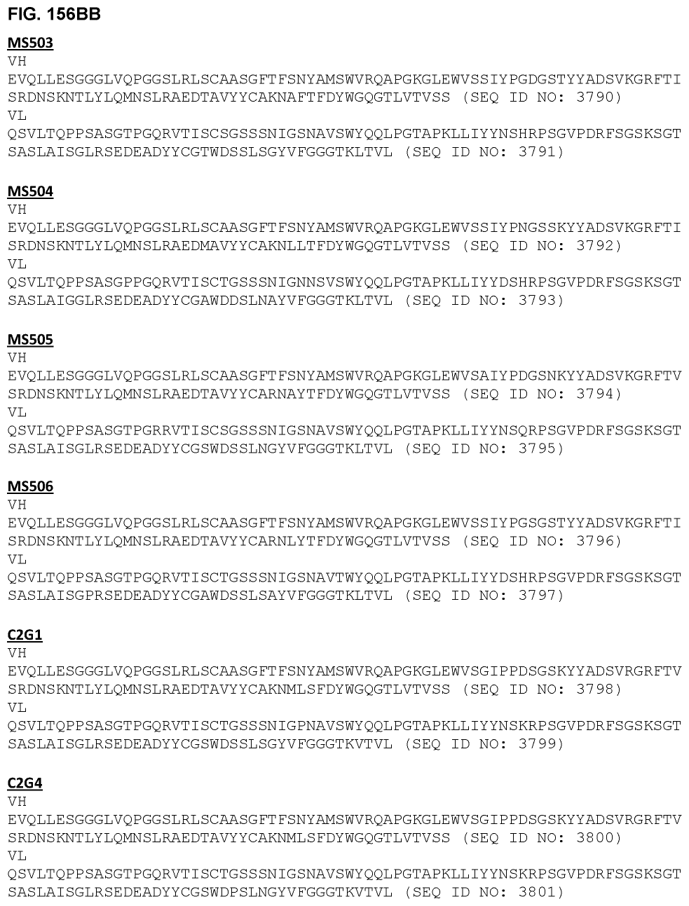

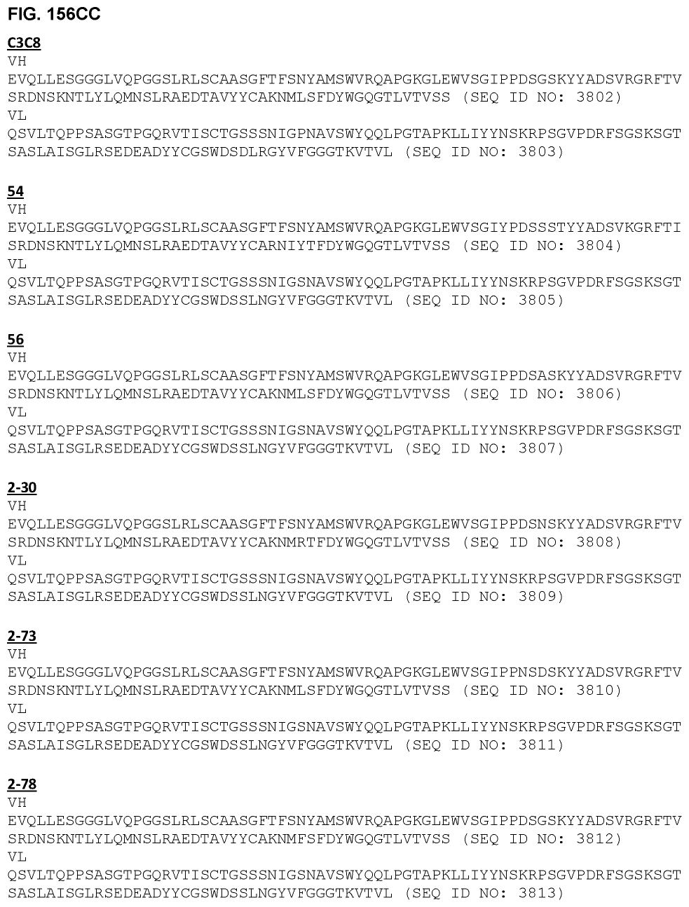

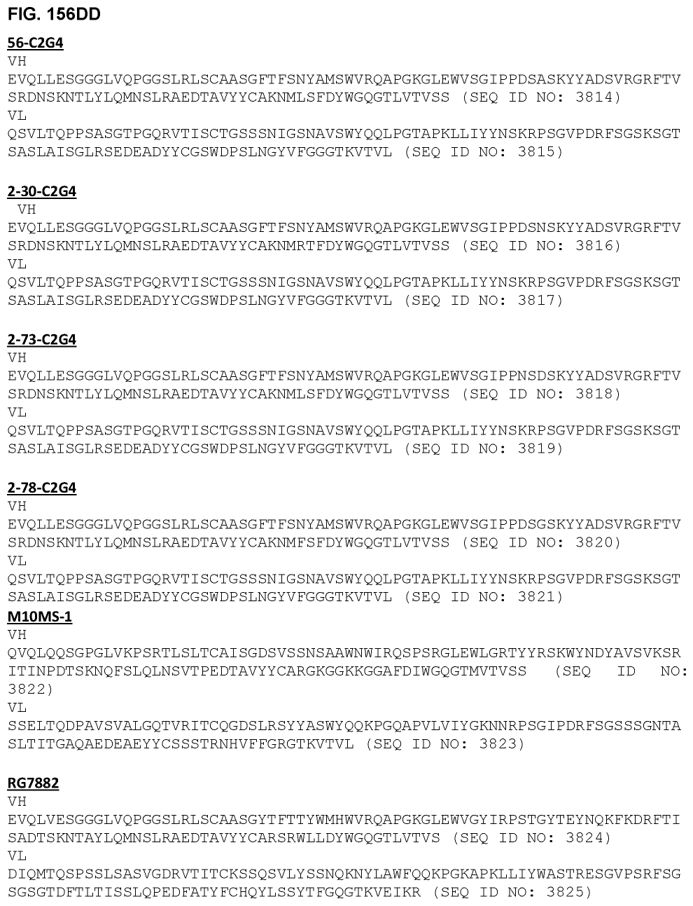

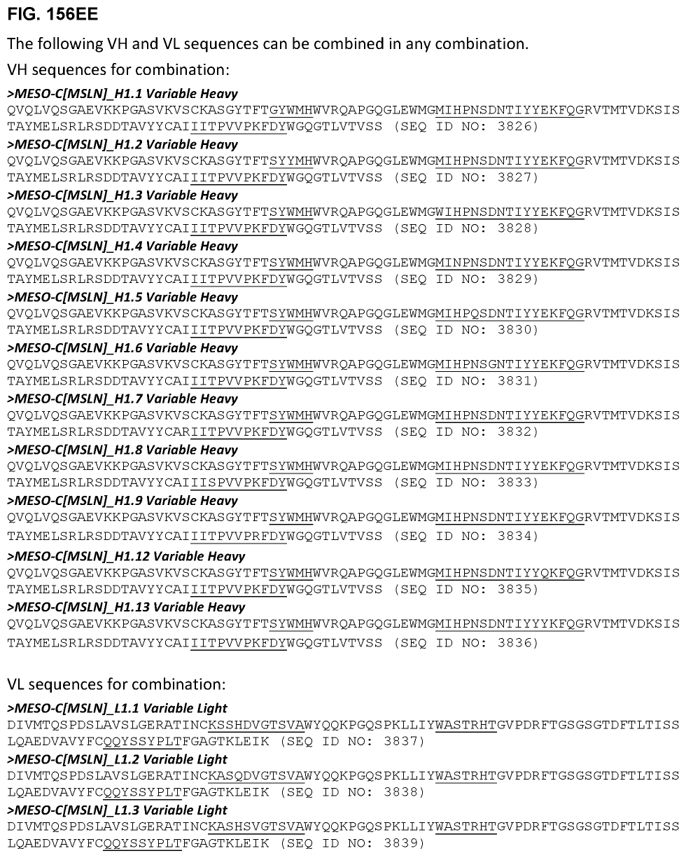

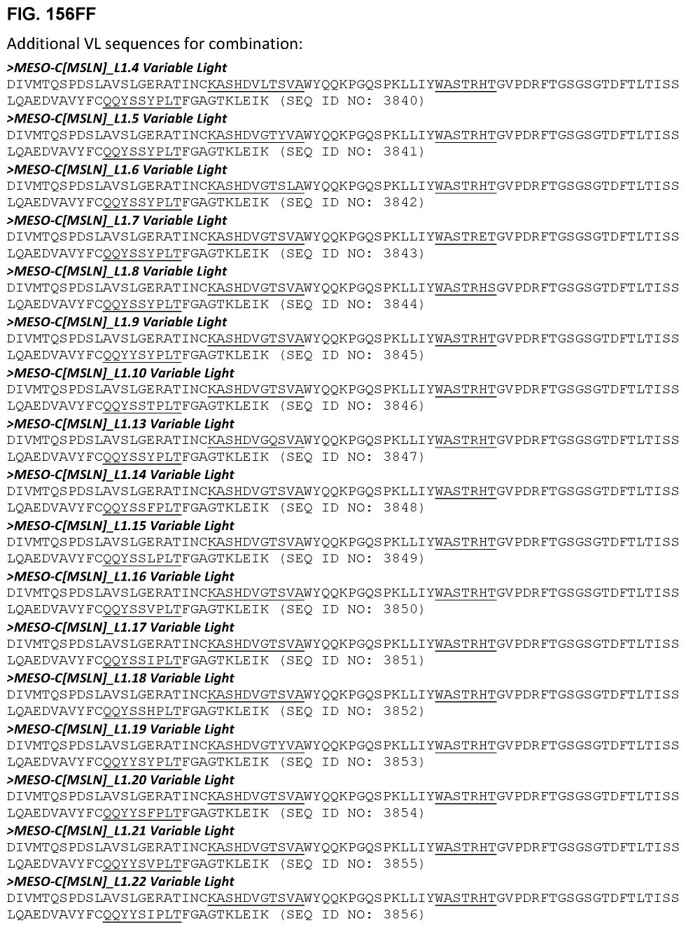

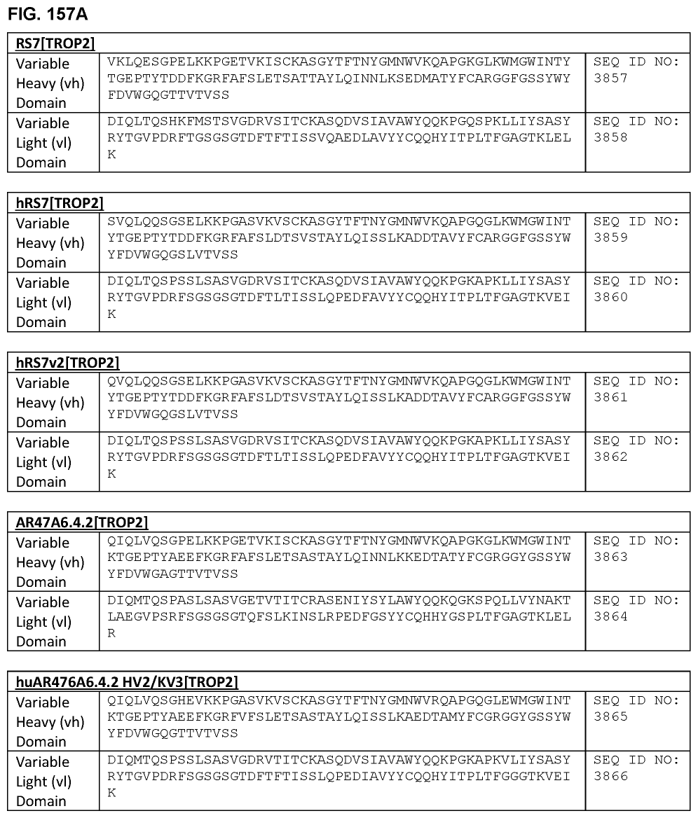

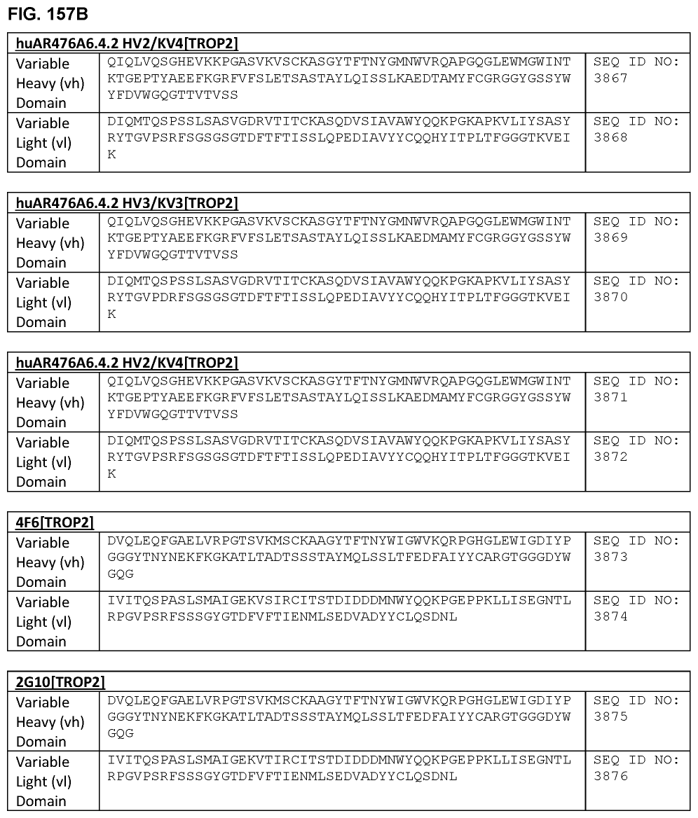

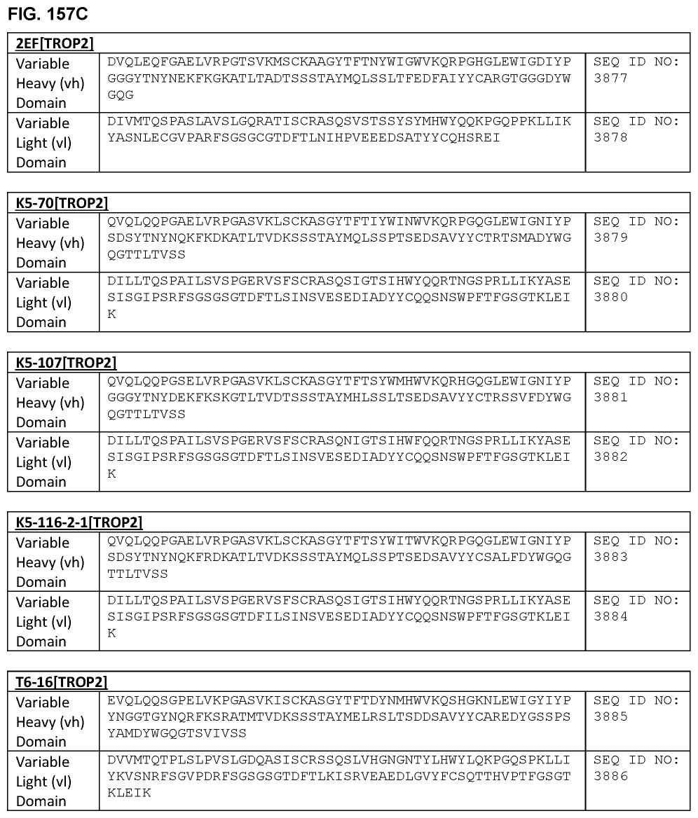

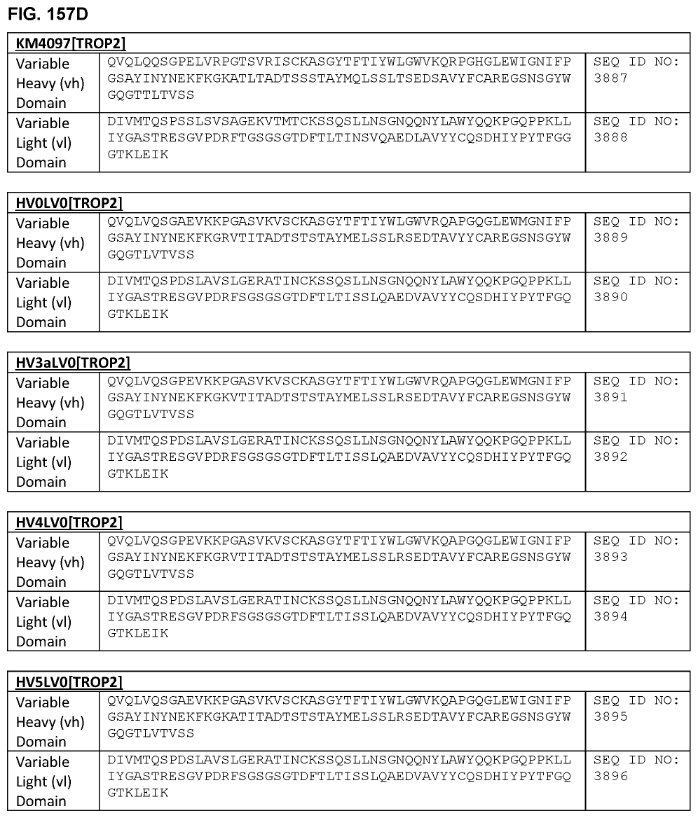

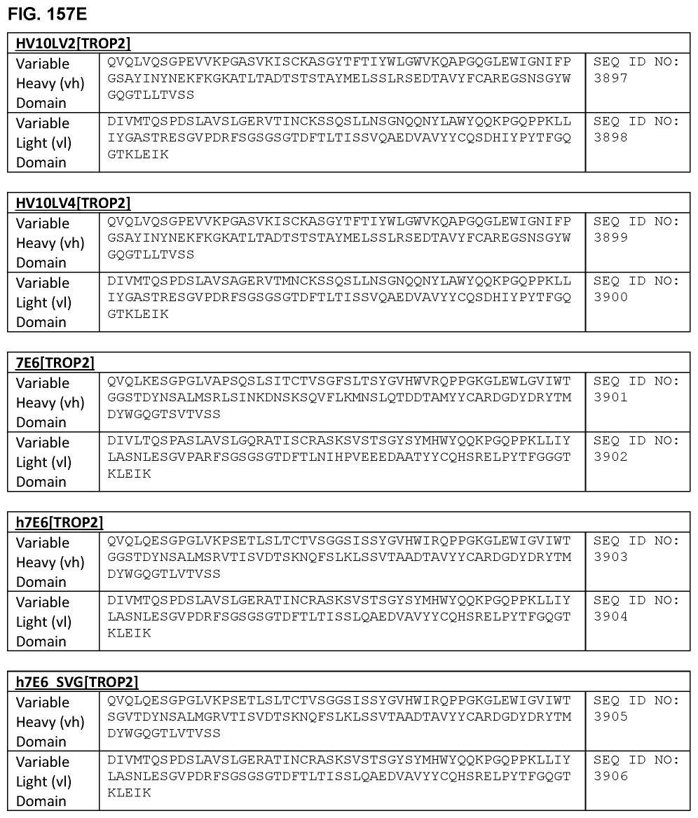

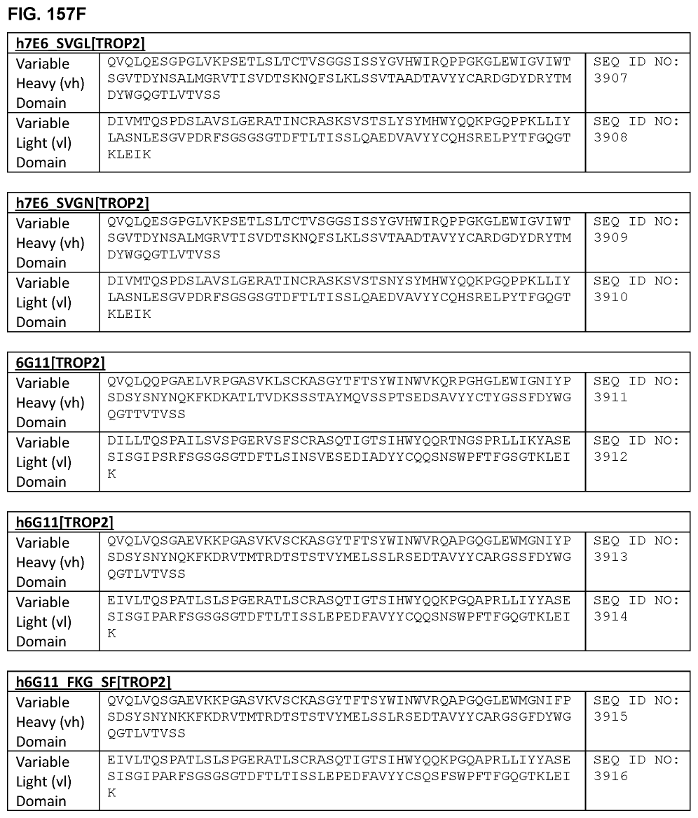

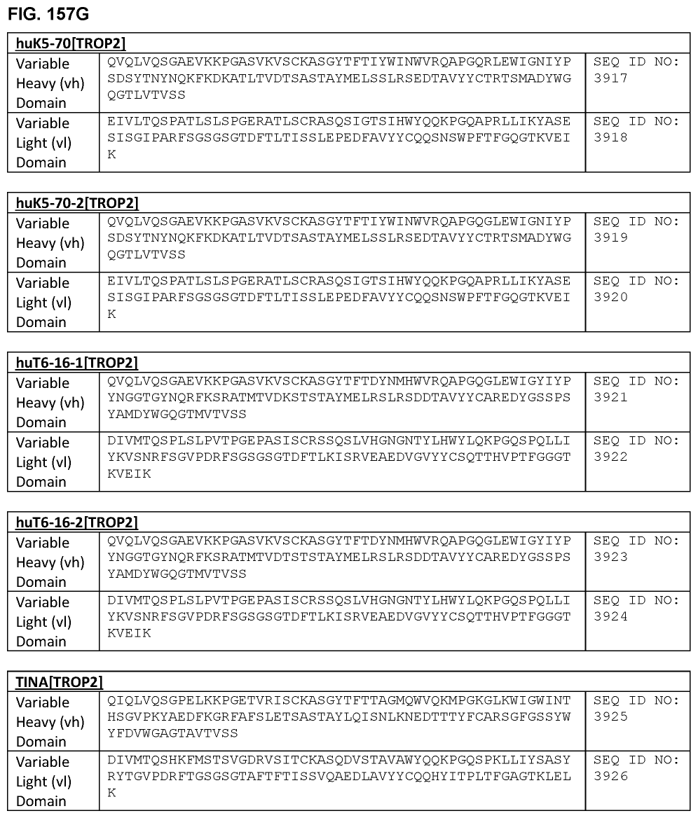

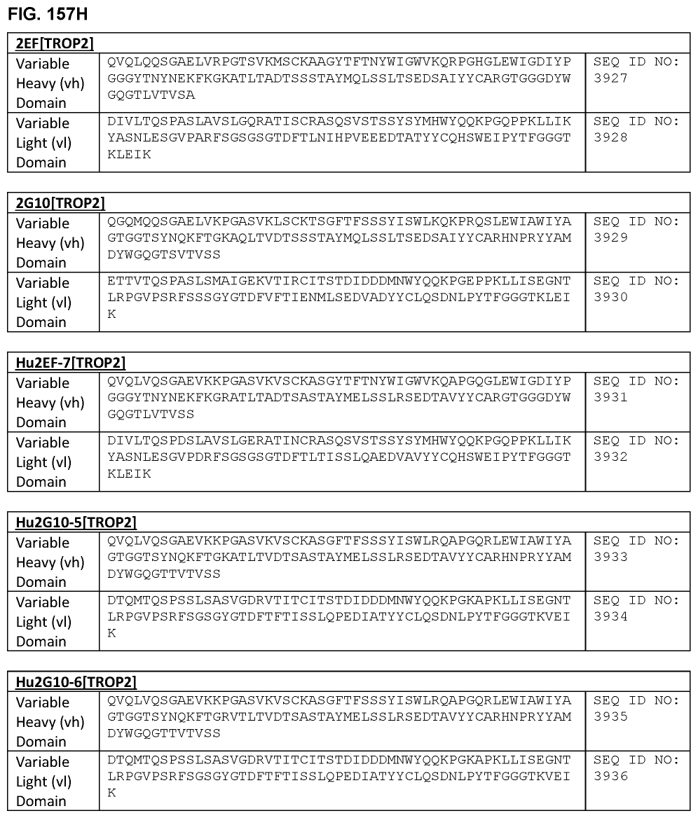

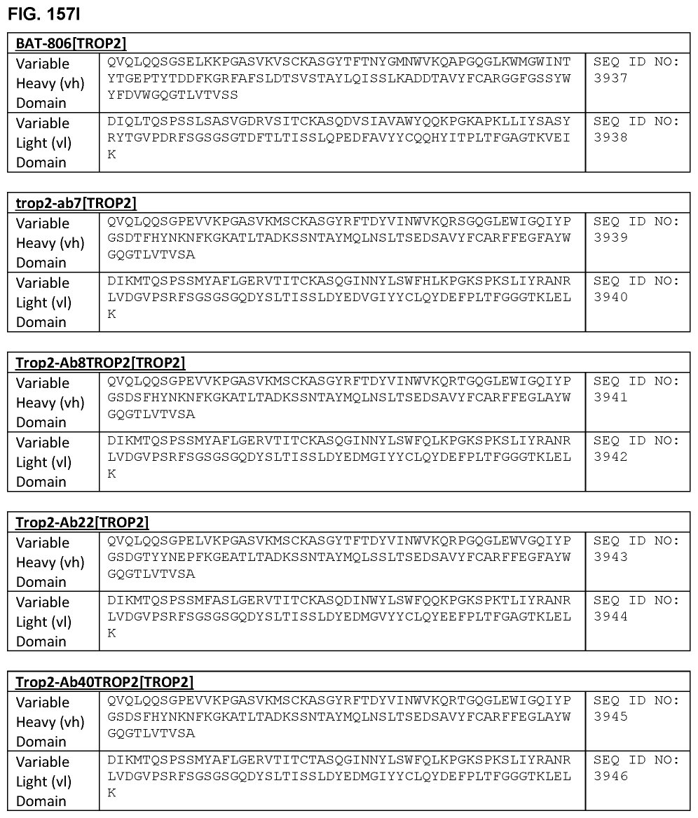

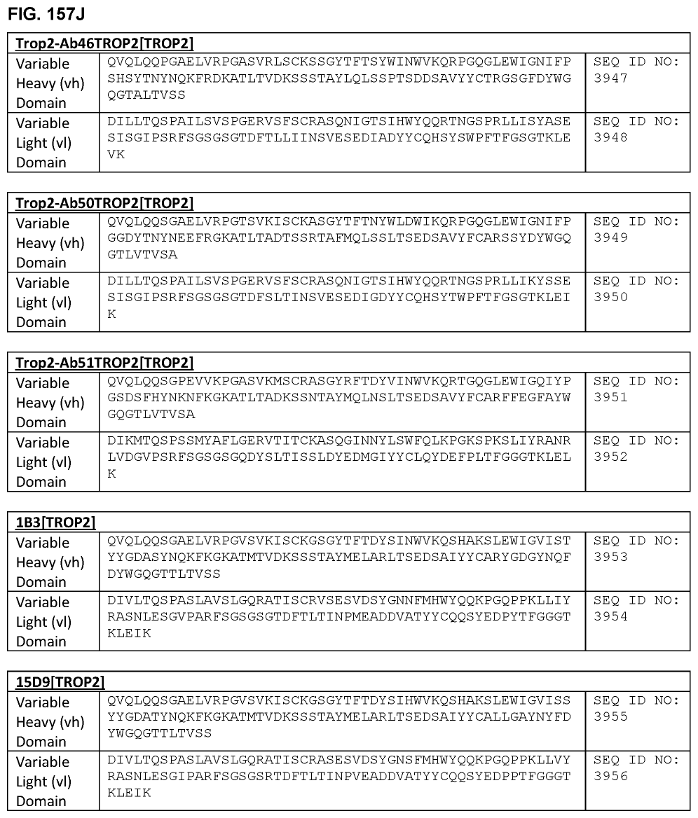

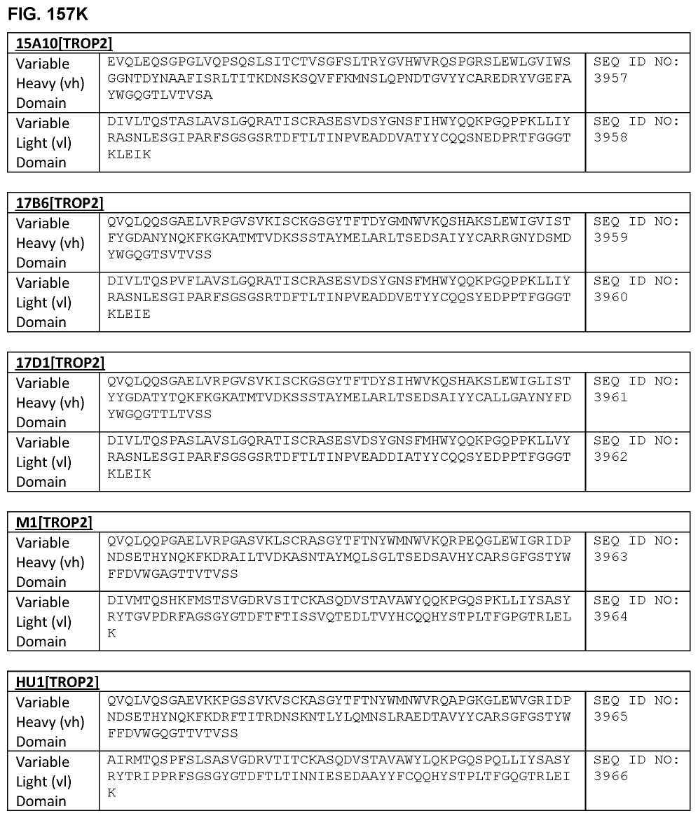

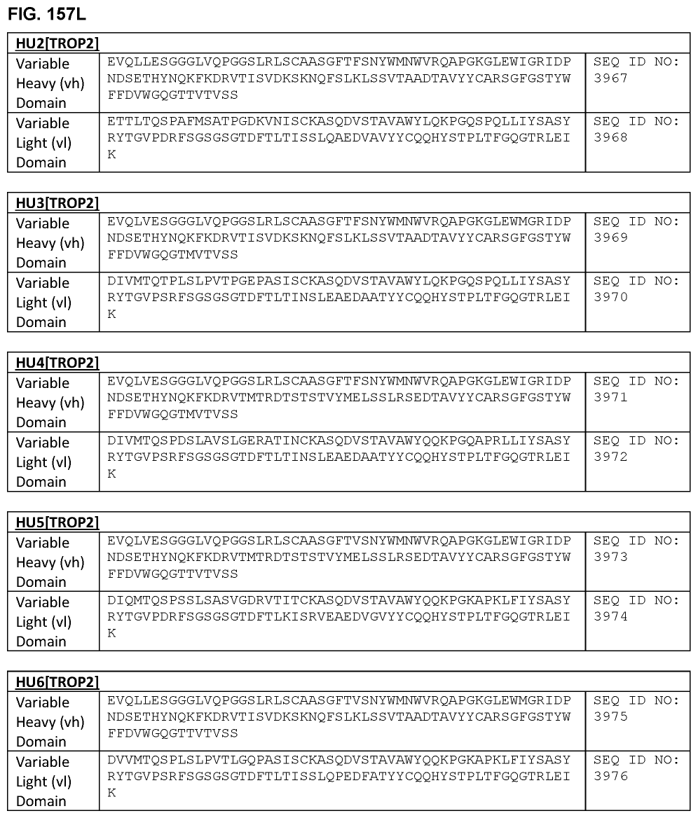

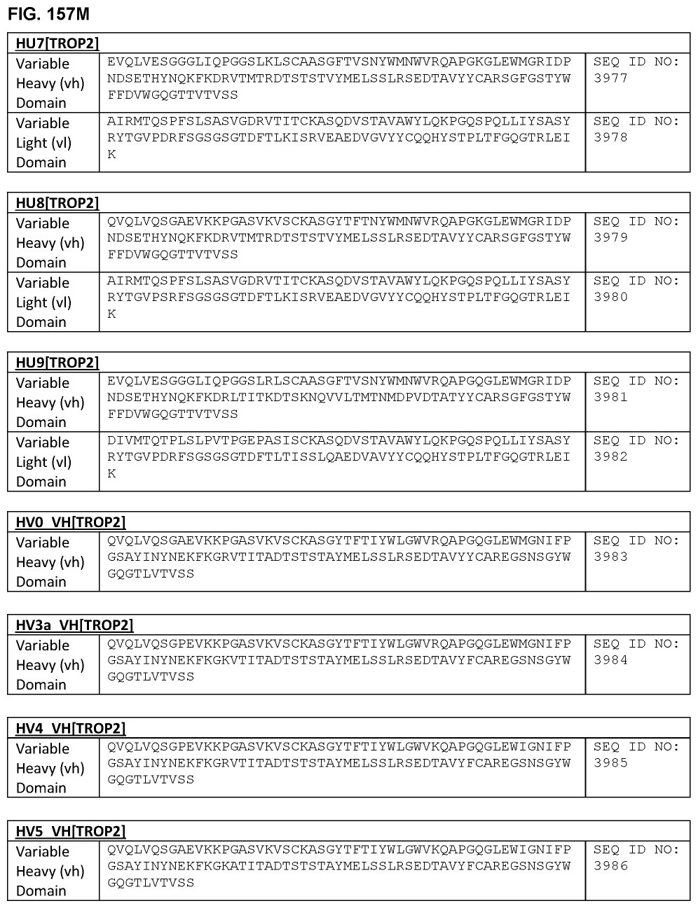

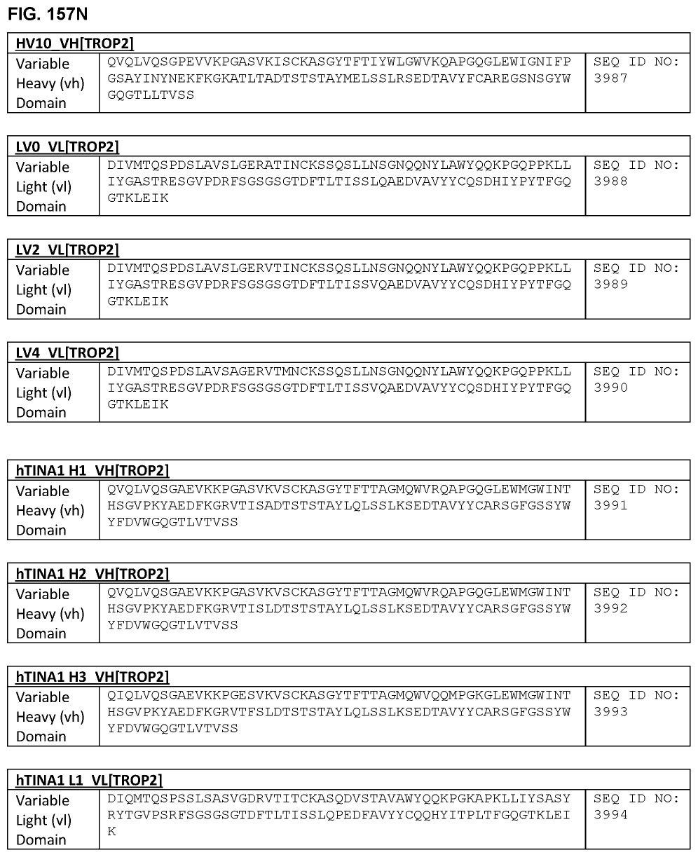

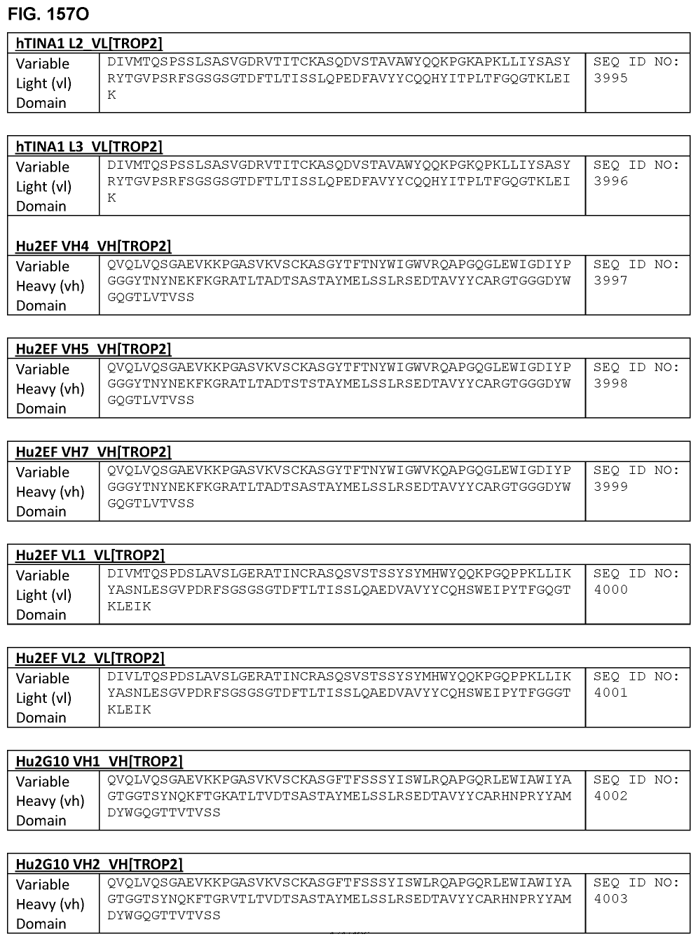

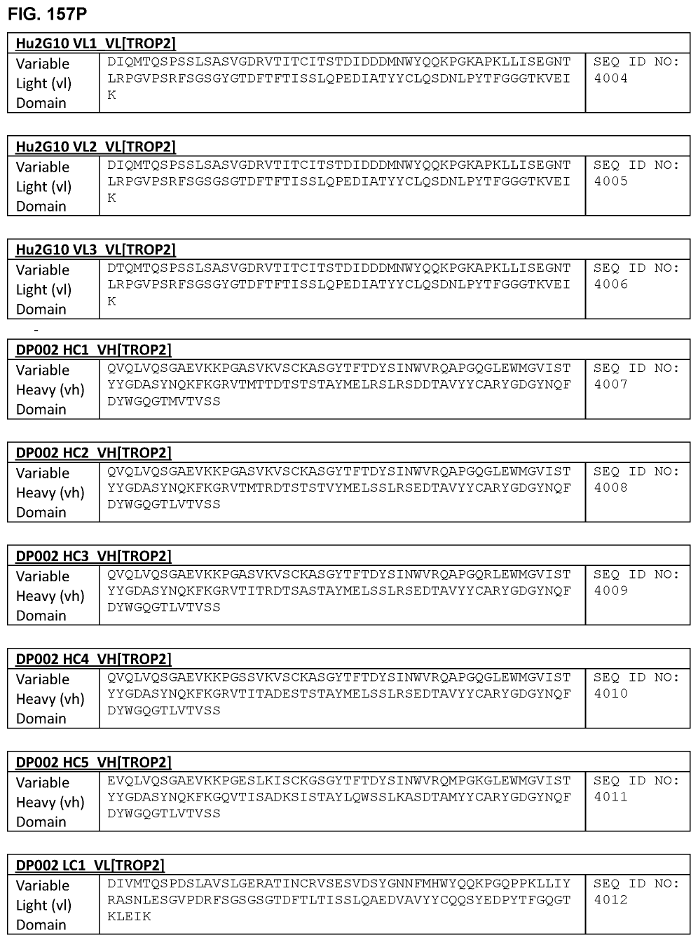

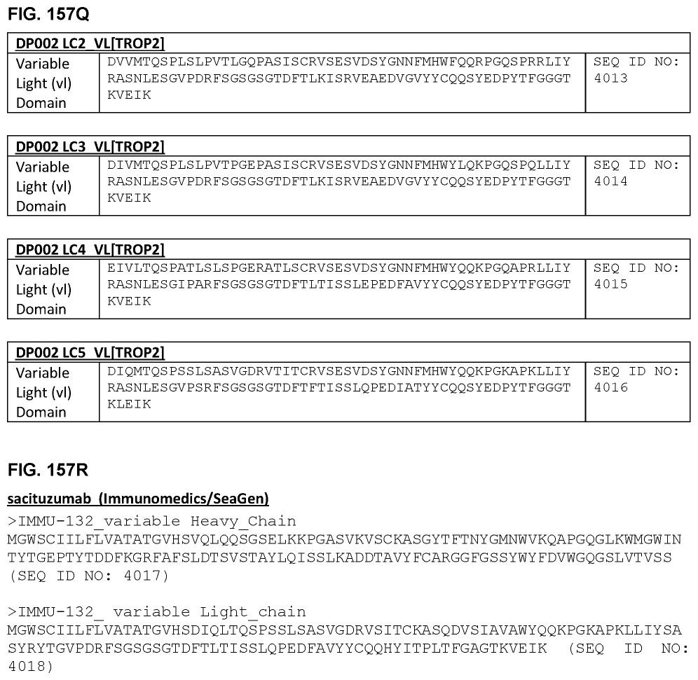

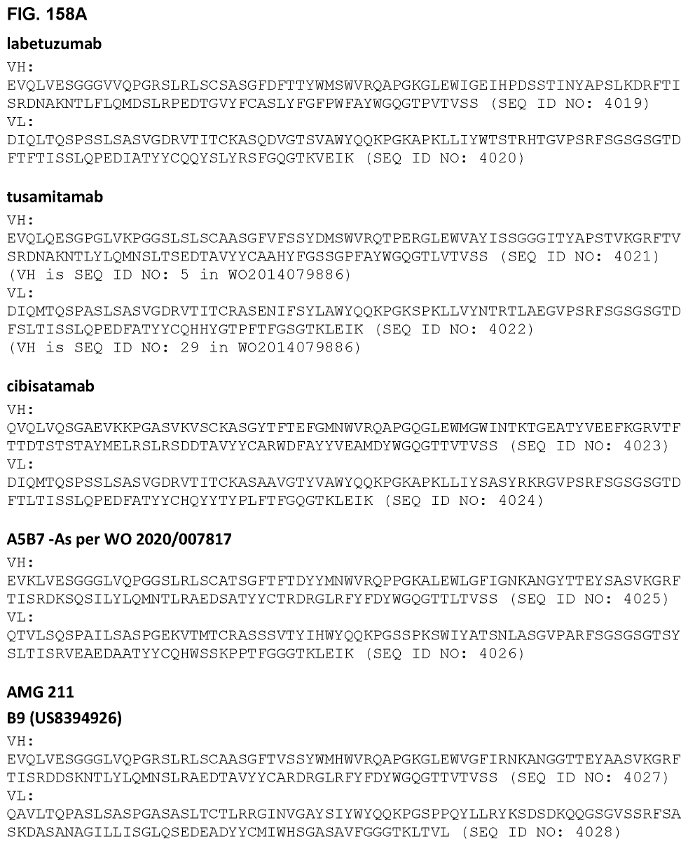

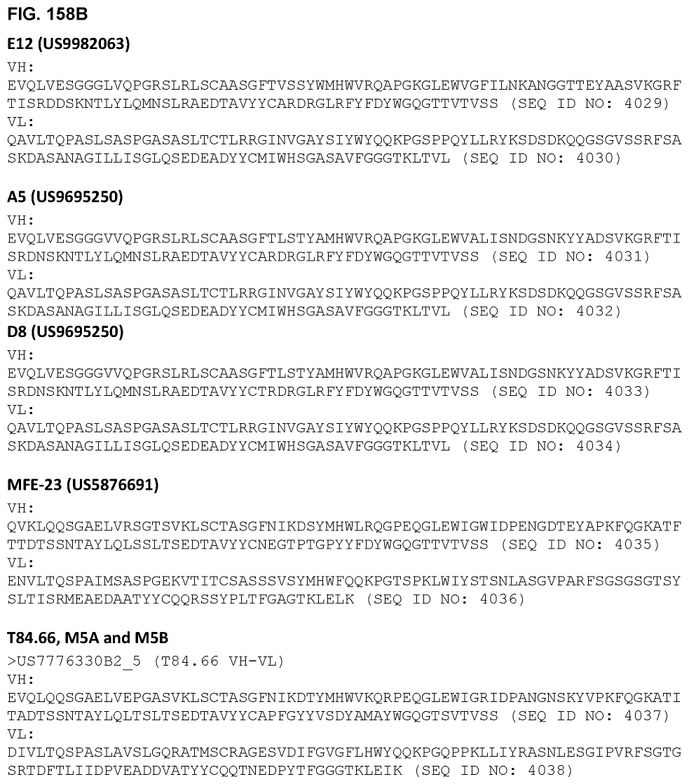

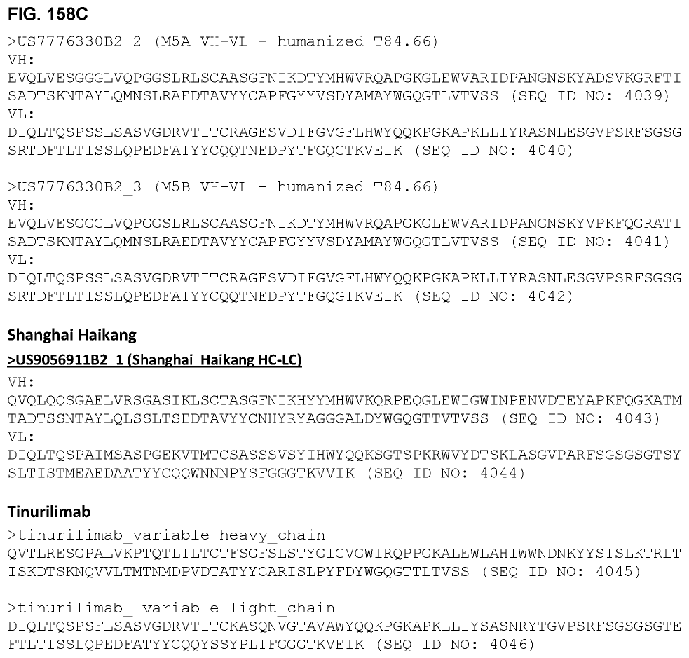

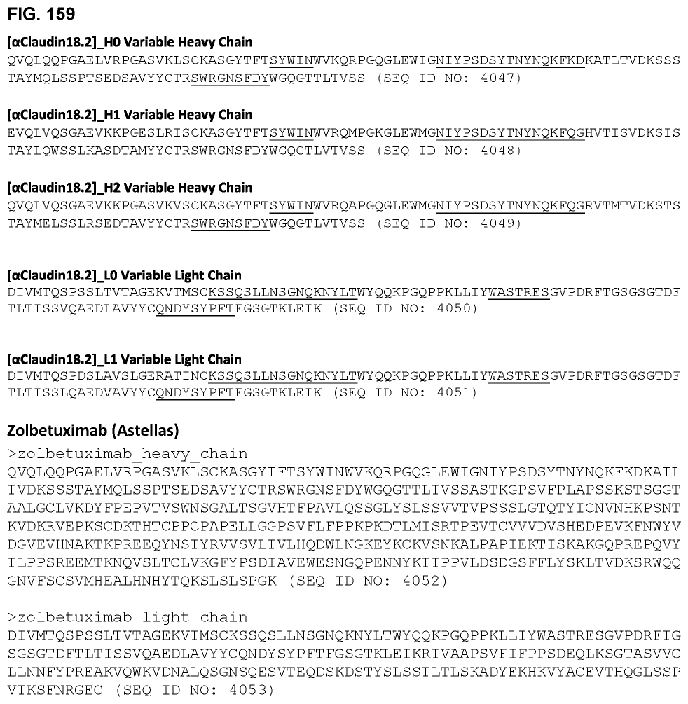

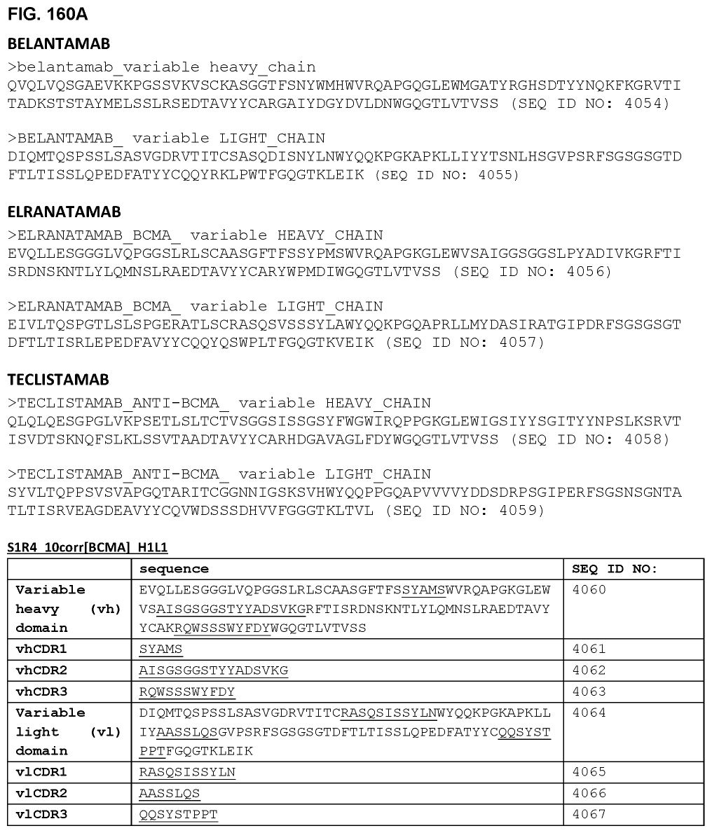

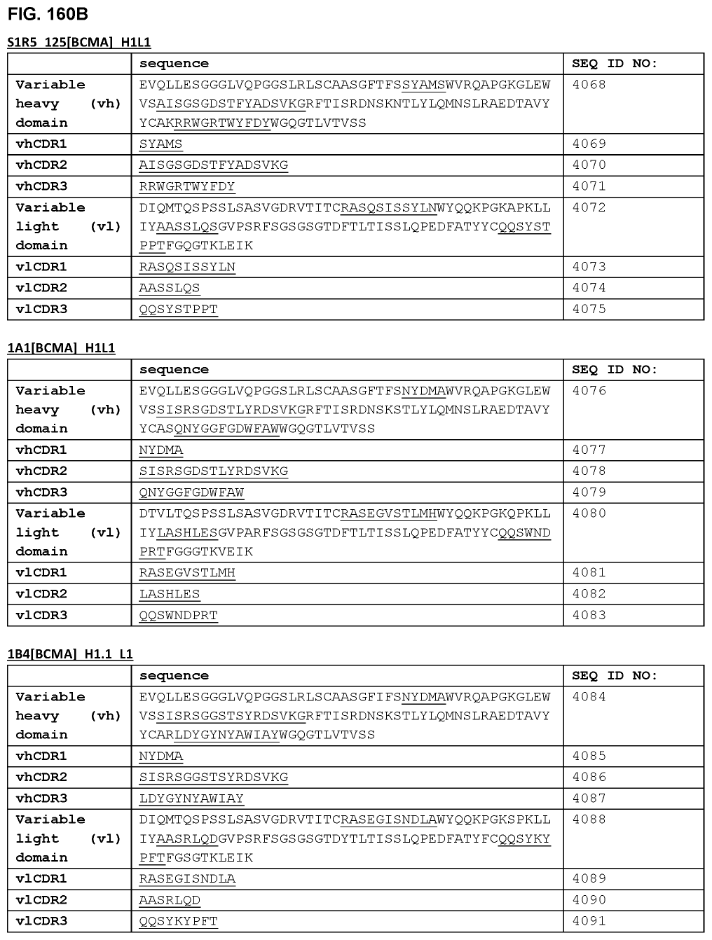

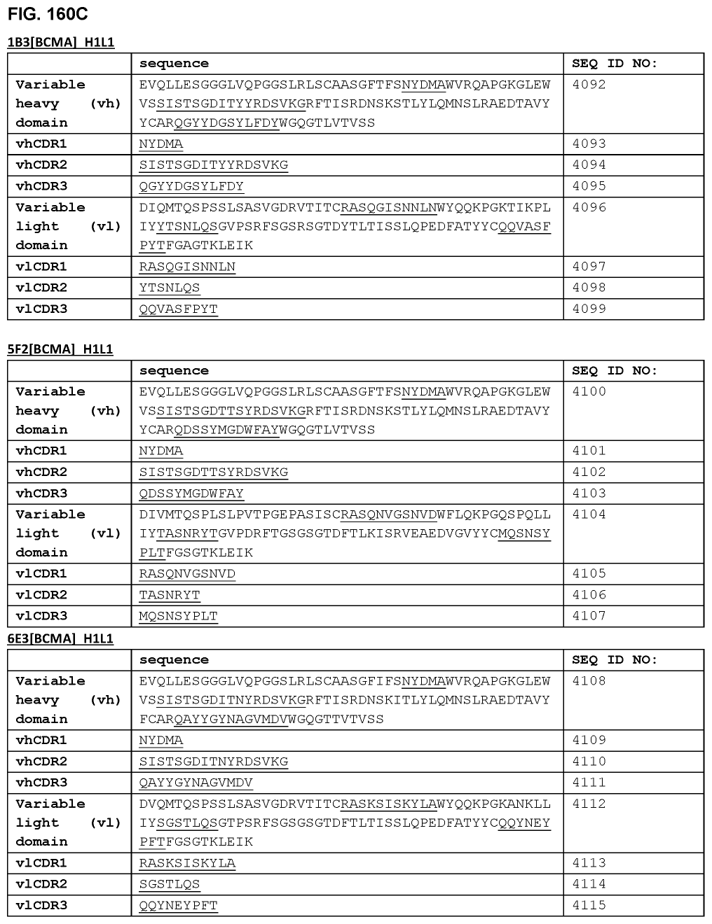

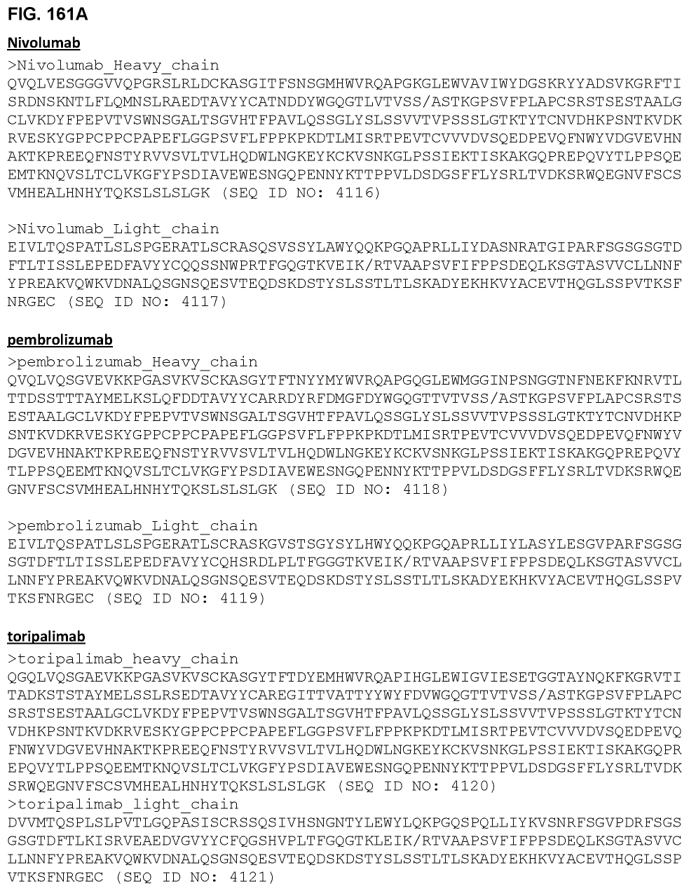

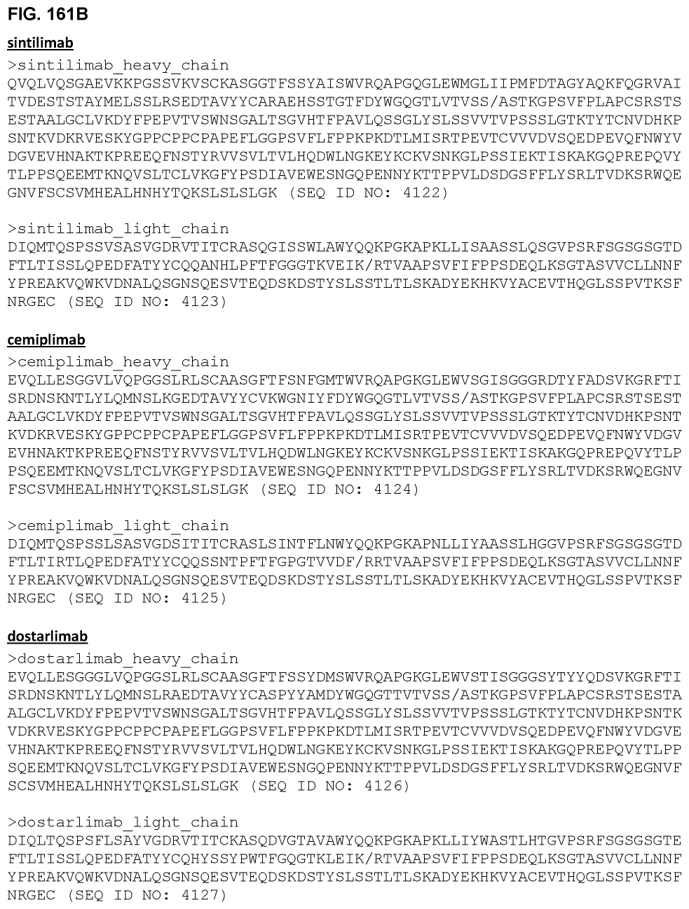

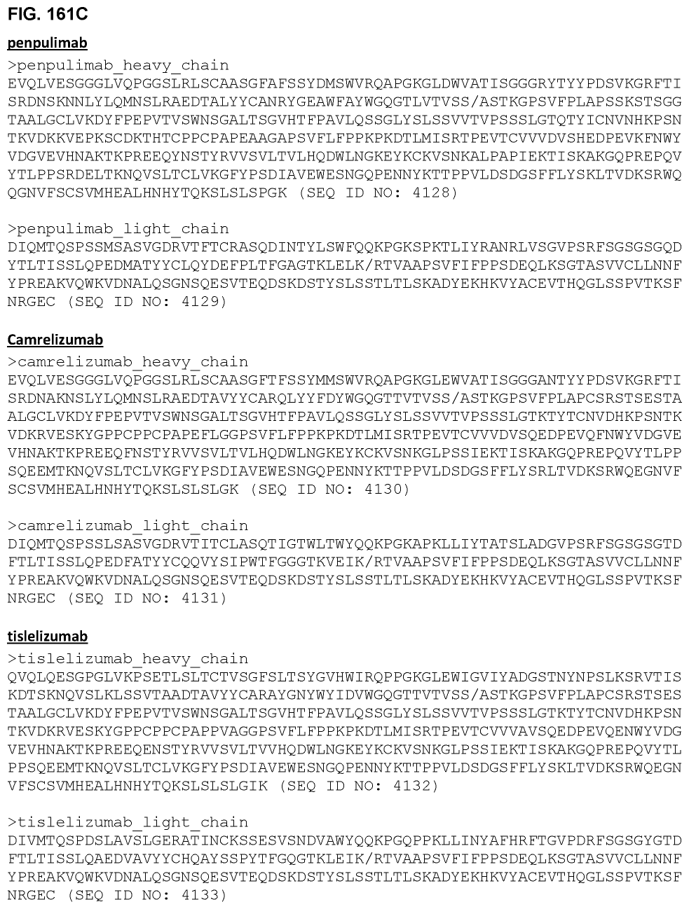

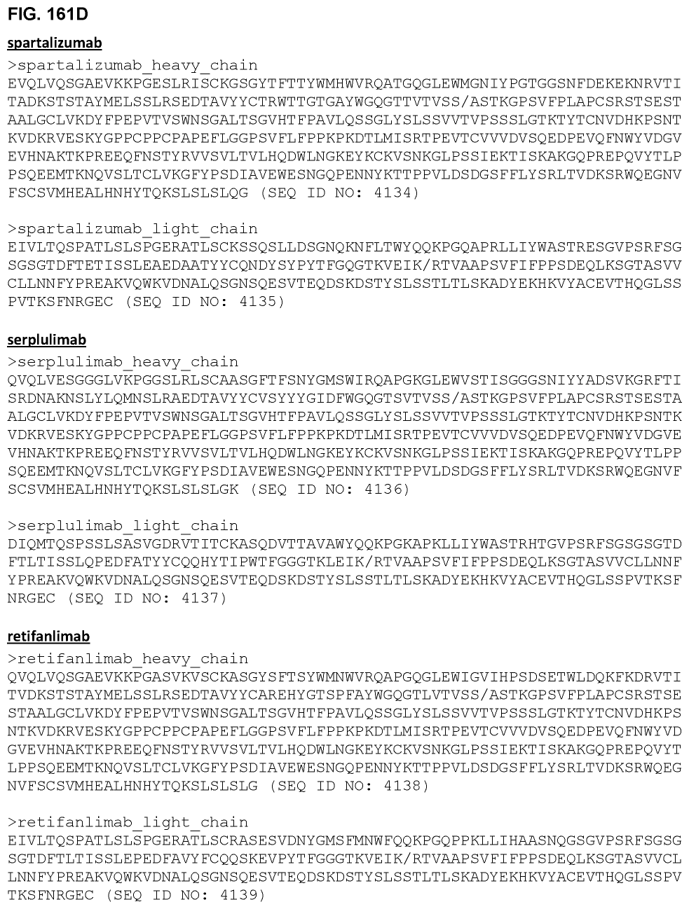

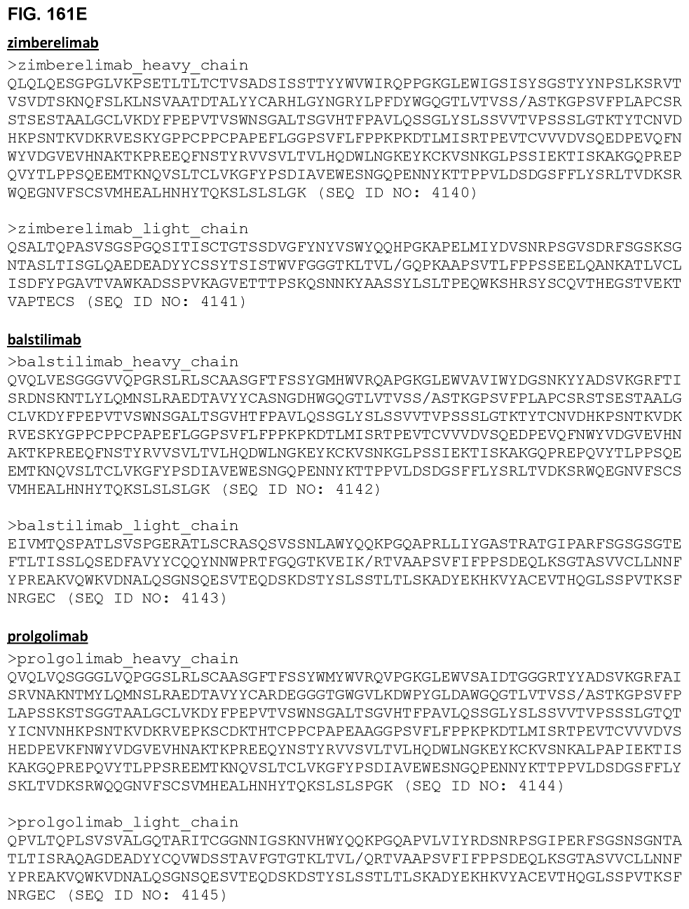

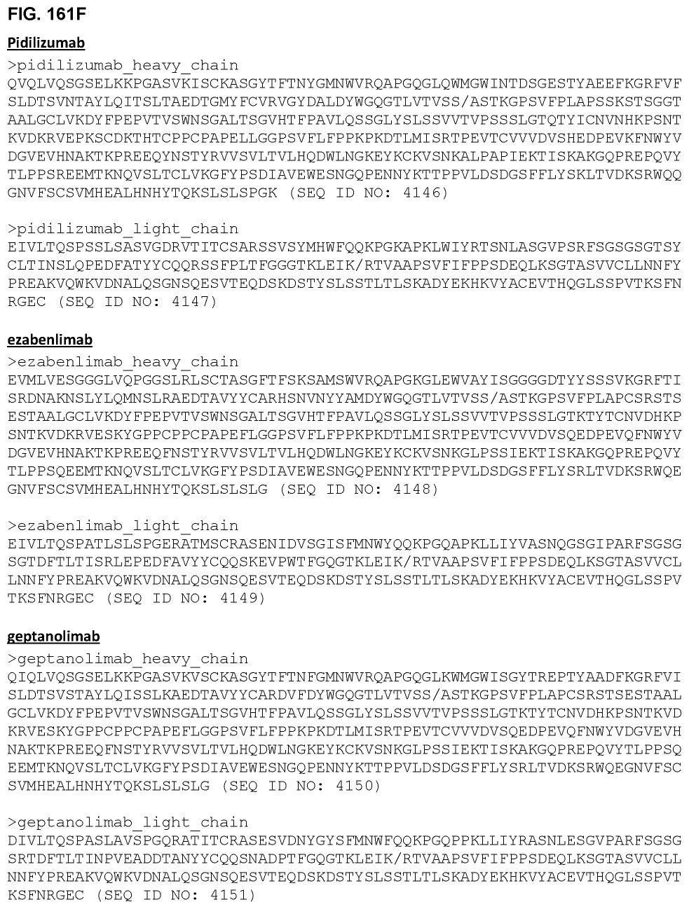

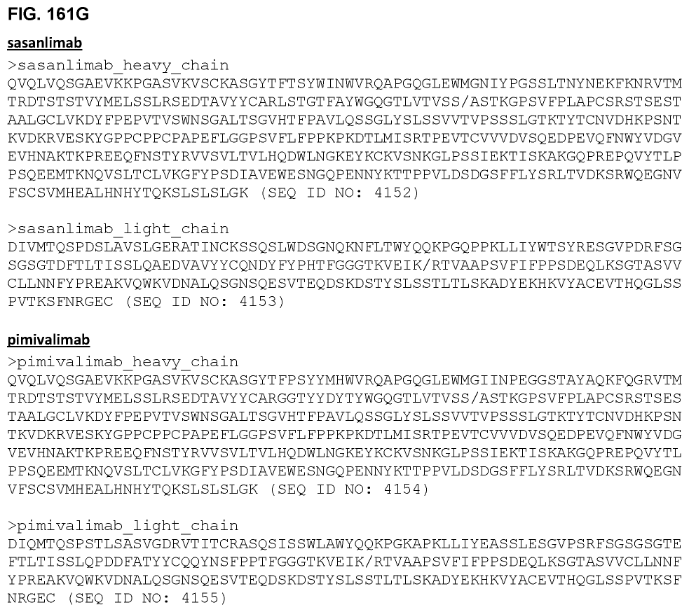

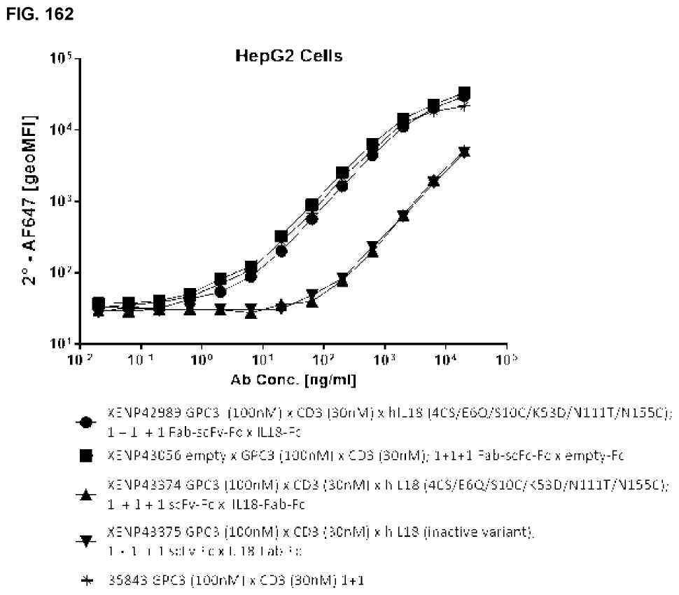

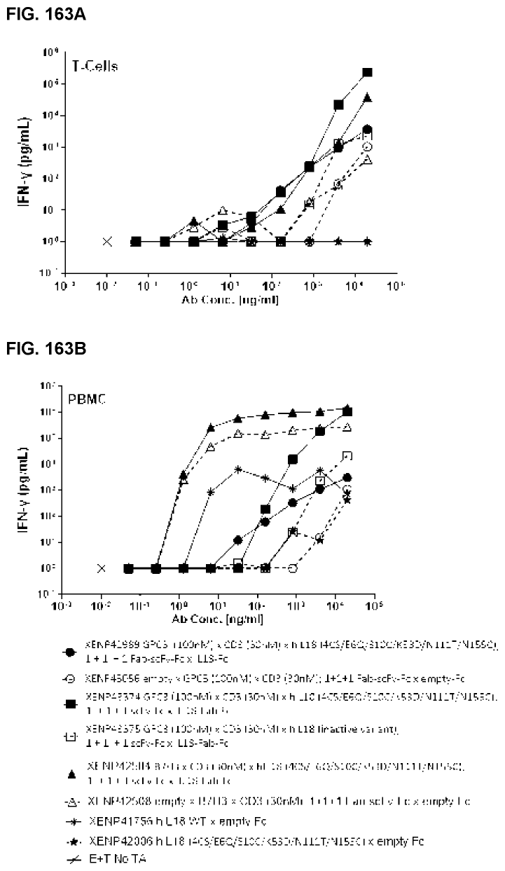

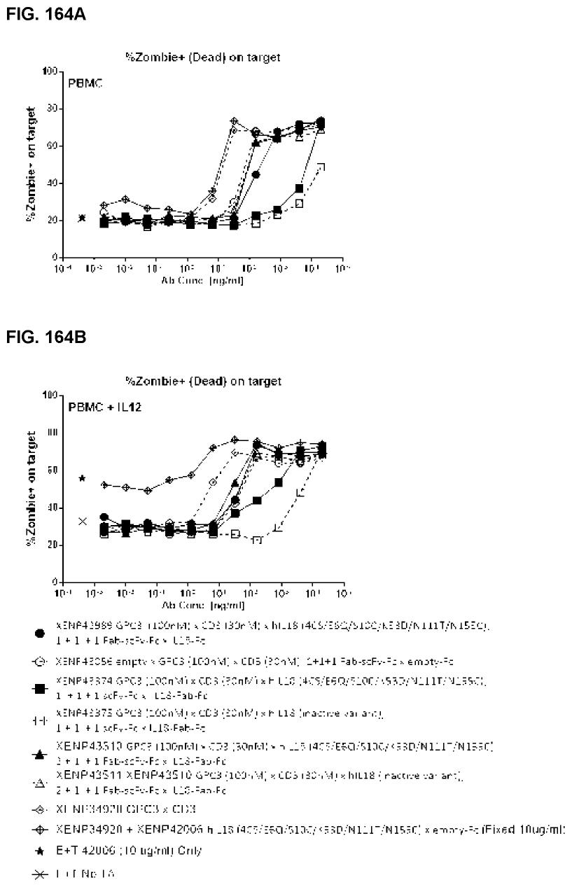

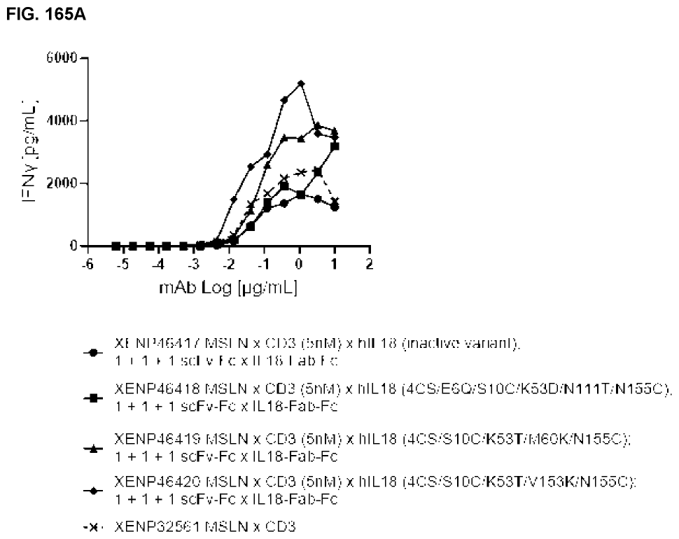

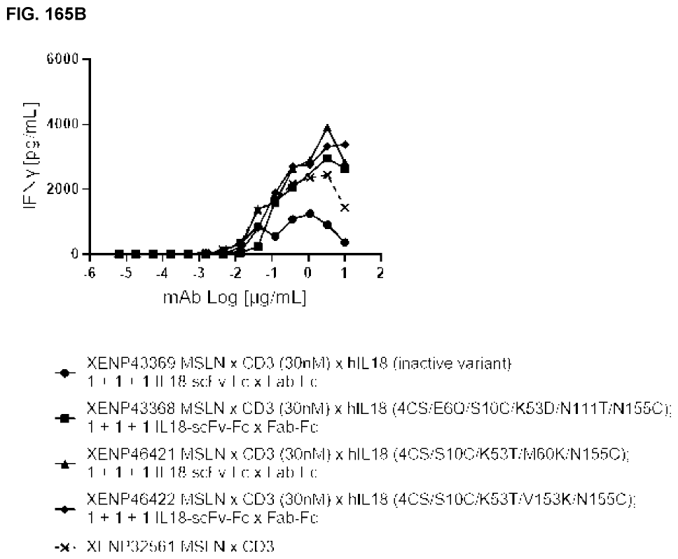

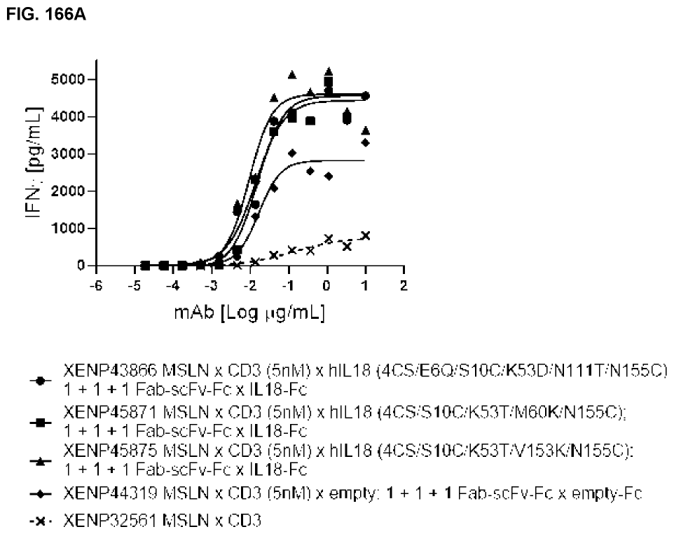

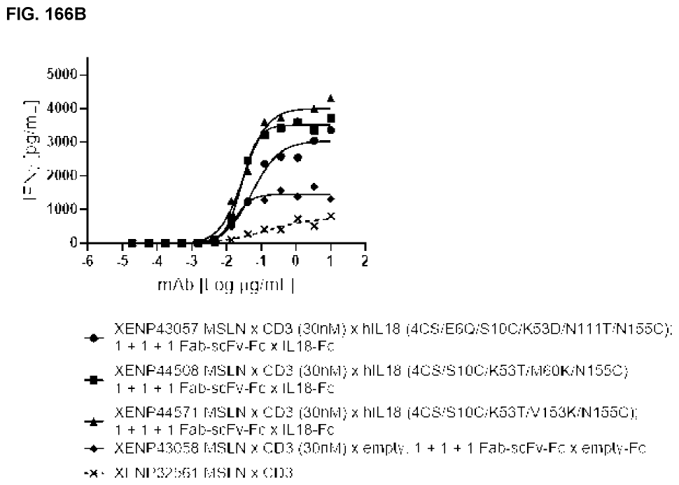

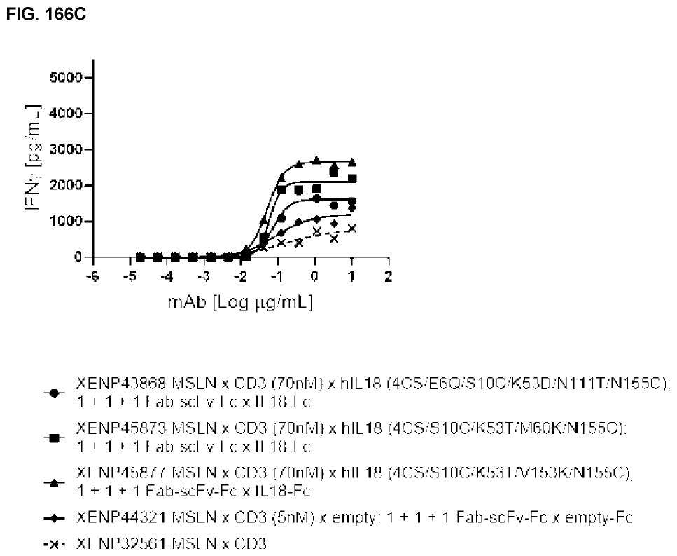

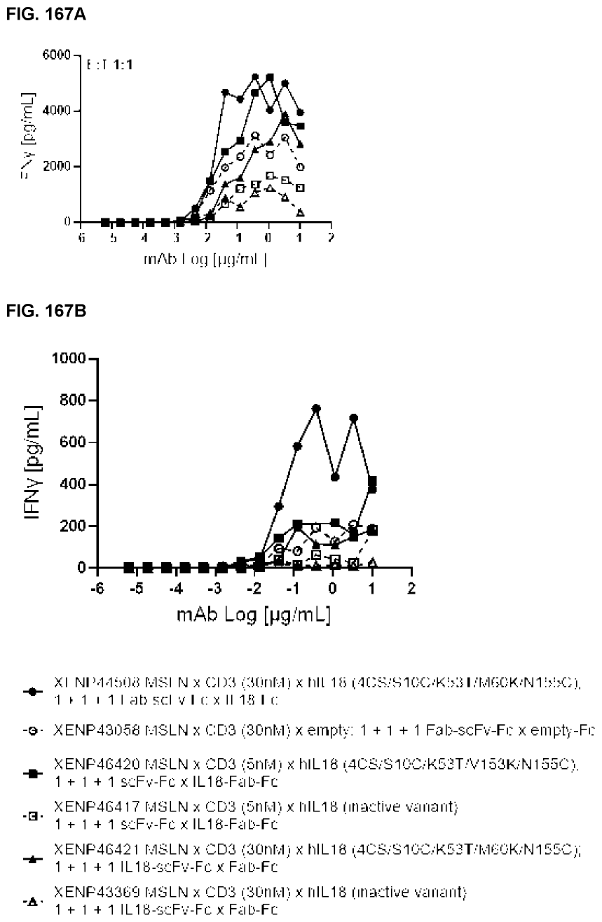

FIG. 10 depicts illustrative CH1 regions which may find use in covalently linking a variable domain to the backbones as depicted in FIG. 9A-E (optionally via a domain linker as depicted in FIG. 8) in the context of IL18×Fab-Fc fusions.

FIG. 11 depicts the “non-Fv” backbone of cognate light chains (i.e., constant light chain) which find use in the IL18×Fab-Fc fusion proteins of the invention.

FIGS. 12A-12B depict illustrative formats for IL18 fusions of the invention. The monovIL18-Fc format (FIG. 12A) comprises a first monomer comprising an IL18 monomer covalently attached to the N-terminus of a first heterodimeric Fc chain (optionally via a domain linker) and a second monomer comprising a complementary second heterodimeric Fc chain that is “Fc-only” or “empty-Fc”. The IL18×Fab-Fc format (FIG. 12B) comprises a first monomer comprising an IL18 monomer covalently attached to the N-terminus of a first heterodimeric Fc chain (optionally via a domain linker), a second monomer comprising a variable heavy (VH) region covalently attached to the N-terminus of a complementary second heterodimeric Fc chain, and a third monomer that is a corresponding light chain that forms a Fab with the second monomer.

FIGS. 13A-13B depict sequences for IL18 production variant comprising C38S, C68S, C76S and/or C127S.

FIG. 14 depicts sequences for IL18 production variants engineered to remove C-terminal aspartic acid.

FIGS. 15A-15B depict sequences for IL18 single substitution affinity variants (Library 1). It should be noted that each of these variants may include additional substitutions such as production variants, affinity variants, and/or stability variants.

FIGS. 16A-16C depict sequences for IL18 single substitution affinity variants (Library 2). It should be noted that each of these variants may include additional substitutions such as production variants, affinity variants, and/or stability variants.

FIG. 17 depicts sequences for IL18 combo substitution affinity variants (Library 2). It should be noted that each of these variants may include additional substitutions such as production variants, affinity variants, and/or stability variants.

FIG. 18 depicts additional affinity variants which may find use in the IL18 fusion proteins of the invention.

FIGS. 19A-19I depict sequences for IL18 stability variants (Library 1). It should be noted that each of these stability variants include the 4CS substitutions, unless reversion to cysteine is explicitly denoted (i.e., S38C, S68C, S76C, and/or S127C). However, it should be noted that the variants at positions other than 38, 68, 76, or 127 may be used independently from the 4CS substitutions. Additionally, it should be noted that each of these variants may include additional substitutions such as production variants, affinity variants, and/or stability variants.

FIGS. 20A-20C depict sequences for IL18 stability variants (Library 2). It should be noted that each of these stability variants include the 4CS substitutions, unless alternative substitution at residues 38, 68, 76, and/or 127 is explicitly denoted (e.g., S38E). However, it should be noted that the substitutions other than C38S, C68S, C76S, and C127S may be used independently from the 4CS substitutions. Additionally, it should be noted that each of these variants may include additional substitutions such as production variants, affinity variants, and/or stability variants.

FIGS. 21A-21I depict the sequences for illustrative IL18 fusions of the monovIL18-Fc format comprising WT human IL18 or human IL18 variants. Slashes (/) indicate the border(s) between IL18 monomer, linkers, and Fc regions. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the monovIL18-Fc sequences may utilize alternative backbones (including, but not limited, to those depicted in FIG. 9A-E).

FIGS. 22A-22DO depict the sequences for illustrative IL18 fusions of the IL18×Fab-Fc format comprising WT human IL18 or human IL18 variants. Slashes (/) indicate the border(s) between IL18 monomer, linkers, and Fc regions. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the IL18×Fab-Fc sequences may utilize alternative backbones (including, but not limited, to those depicted in FIG. 9A-E) or alternative variable heavy and variable light domains.

FIGS. 23A-23B depict A) chromatogram illustrating purification part 2 of XENP30792 (anion exchange chromatography following protein A chromatography), and the heterogeneity of pre-purified material and material from peak B isolated from anion exchange separation as depicted in FIG. 23A by B) analytical size-exclusion chromatography with multi-angle light scattering (aSEC-MALS). FIG. 23B also depicts the molecular weight of protein species as determined by multi-angle light scattering.

FIGS. 24A-24B depict A) chromatogram illustrating purification part 2 of XENP31296 (anion exchange chromatography following protein A chromatography), and the heterogeneity of pre-purified material and purity of material from peak B isolated from anion exchange separation as depicted in FIG. 24A by B) analytical size-exclusion chromatography with multi-angle light scattering (aSEC-MALS). FIG. 24B also depicts the molecular weight of protein species as determined by multi-angle light scattering.

FIGS. 25A-25B depict A) chromatogram illustrating purification part 2 of XENP37827 (anion exchange chromatography following protein A chromatography), and the purity material from peak B isolated from anion exchange separation as depicted in FIG. 25A by B) analytical size-exclusion chromatography with multi-angle light scattering (aSEC-MALS).

FIG. 25B also depicts the molecular weight of protein species as determined by multi-angle light scattering.

FIGS. 26A-26B depict A) dose dependent activation of KG-1 cells (as indicated by PD-L1 expression) by recombinant human TL18 and B) dose dependent neutralization of TL18 activity on KG-1 cells (as indicated by PD-1 expression) by TL18BP in the presence of 0.86 nM (100 ng/ml) recombinant human IL18.

FIG. 27 depicts activation of KG-1 cells (as indicated by induction of PD-L1 expression) by TL18 affinity variants (in the context of IL18×Fab-Fc fusions having a silent Fv). The data show that a number of substitutions enabled reduced activation potency in comparison to WT-4CS variant.

FIGS. 28A-28S depict activation of KG-1 cells (as indicated by induction of PD-L1 expression) by TL18 affinity variants (in the context of IL18×Fab-Fc fusions having a silent Fv) in the absence of L18BP (solid symbols/solid lines) or in the presence of 100 ng/ml TL18BP (open symbol/dotted lines). The data show that a number of substitutions enabled reduced activation potency in comparison to WT-4CS variant. For most of the variants, incubation with TL18BP shifts the activation potency; however, XENP38865 having the K53A appear minimally impacted by TL18BP indicating that the K53A substitution reduces binding affinity and sink by IL18BP.

FIG. 29 shows dose dependent inhibition of KG-1 cell activation (as indicated by induction of PD-L1 expression) by IL18BP and fixed concentration (10 nM) TL18 affinity variants (in the context of IL18×Fab-Fc fusions having a silent Fv). The data show that some of the variants were very sensitive to IL18BP inhibition. However, XENP38865 having the K53A substitution was much less susceptible to IL18BP inhibition (as indicated by higher IC50 value of 88.73 nM in comparison to IC50 value of 5.503 for XENP37827 having WT-4CS IL18).

FIG. 30 shows dose dependent inhibition of KG-1 cell activation (as indicated by induction of PD-L1 expression) by IL18BP and fixed concentration (10 nM) IL18 affinity variants (in the context of IL18×Fab-Fc fusions having a silent Fv).

FIG. 31 depicts BLI-response by IL18×Fab-Fc fusions comprising IL18 affinity variants (Library 2) for IL18R1, IL18BP, and IL18R1×IL18RAP heterodimer as determined by Octet. The data show that the K53D and K53N single substitution variants exhibited weaker binding response to IL18R1 and IL18R1×IL18RAP but also no binding response to IL18BP. Several other substitutions at K53 also enabled weakened binding for both IL18R1 and IL18BP, albeit not to the same level of weakened IL18BP binding as K53D and K53N; S55N and S55Q enabled weakened binding to IL18R1 and IL18BP, although at the expense of stability (as depicted in FIG. 35); and M41K enabled enhanced binding for IL18R1.

FIG. 32 depicts activation of KG-1 cells (as indicated by induction of PD-L1 expression) by IL18 affinity variants (in the context of IL18×Fab-Fc fusions having a silent Fv) in the absence of IL18BP.

FIG. 33 depicts activation of KG-1 cells (as indicated by induction of PD-L1 expression) by IL18 affinity variants (in the context of IL18×Fab-Fc fusions having a silent Fv) in the absence of IL18BP. The M51K variant appears to improve IL18 response.

FIG. 34 depicts activation of KG-1 cells (as indicated by induction of PD-L1 expression) by IL18 affinity variants (in the context of IL18×Fab-Fc fusions having a silent Fv) in the absence of IL18BP.

FIG. 35 depicts activation of KG-1 cells (as indicated by induction of PD-L1 expression) by IL18 affinity variants (in the context of IL18×Fab-Fc fusions having a silent Fv) in the absence of IL18BP.

FIG. 36 depicts melting temperature of IL18 affinity variants (Library 1) in the context of IL18×Fab-Fc fusions having a silent Fv.

FIG. 37 depicts melting temperature of IL18 affinity variants (Library 2) in the context of IL18×Fab-Fc fusions having a silent Fv and RP variants. K53N, P57A, and P57E increased Tm1 respectively by 5, 9.5, and 12° C. Several substitutions, including alternative substitutions as the aforementioned positions (e.g., K53I), decreased Tm1 by as much as 11.5° C.

FIGS. 38A-38B depict sequence alignment of IL18 sequences from various species (SEQ ID NOS: 1369-1379).

FIGS. 39A-39B depict melting temperature of IL18 stability variants (Library 1) in the context of IL18×Fab-Fc fusions having a silent Fv and RP variants.

FIG. 40 depicts melting temperature of IL18 stability variants (Library 2) in the context of IL18×Fab-Fc fusions having a silent Fv and RP variants.

FIGS. 41A-41C depict sequences for Further Variants (Library 1) which explored combinations of favorite variants from Stability Variants (Libraries 1 and 2). It should be noted that each of these variants may include additional substitutions such as production variants, affinity variants, and/or additional stability variants. Additionally, while these variants were produced with delD193 as they were assessed in the context of His-tagged molecules, these IL18 variants may be produced without delD193.

FIGS. 42A-42D depict sequences for Further Variants (Library 2) which explored further substitutions at positions identified from Affinity Variants (Libraries 1 and 2) as well as at newly identified positions S36 and D132. It should be noted that each of these variants may include additional substitutions such as production variants, additional affinity variants, and/or stability variants.

FIGS. 43A-43B depict sequences for Further Variants (Library 3) which explored combinations of favorite affinity variants with S10C/N155C. It should be noted that each of these variants may include additional substitutions such as production variants, affinity variants, and/or stability variants.

FIGS. 44A-44C depict sequences for Further Variants (Library 4) which explored combinations of favorite affinity variants. In particular in certain combinations, variants enhanced in IL18 receptor binding were combined with K53D to restore its reduced IL18 receptor binding. It should be noted that each of these variants may include additional substitutions such as production variants, affinity variants, and/or stability variants.

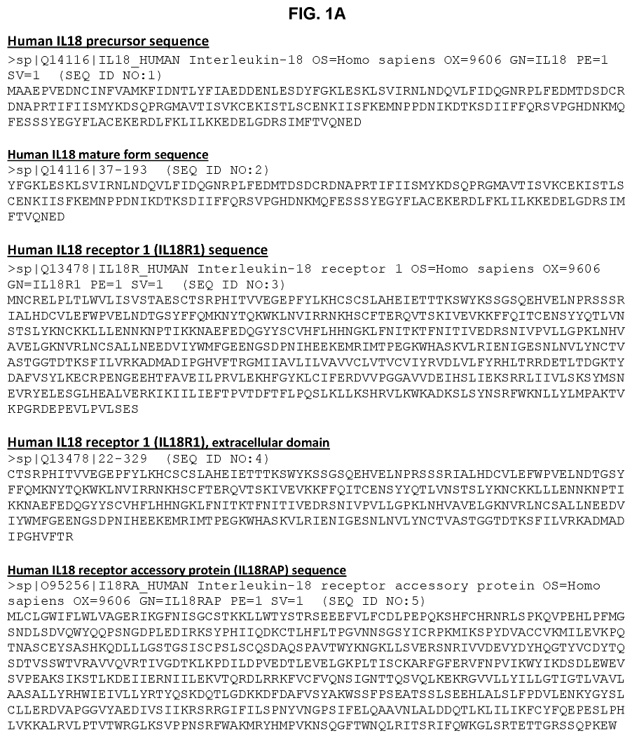

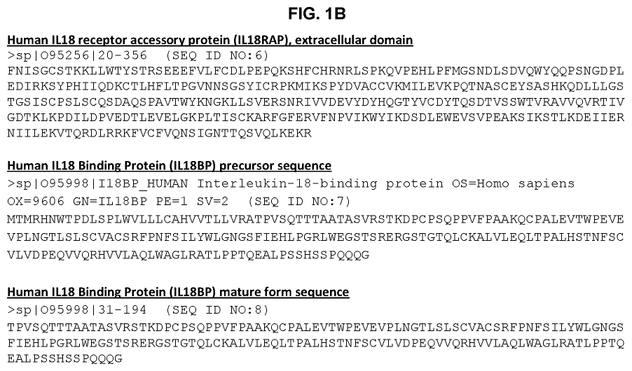

FIG. 45 depicts IL18 positions and substitutions for modulating IL18 binding affinity for IL18 receptors and/or IL18BP. It should be noted that these substitutions can be combined with any other substitutions such as production variants, additional affinity variants, and/or stability variants. The numbering is in the context of Human IL18 mature form sequence as depicted in FIG. 1.

FIG. 46 depicts IL18 positions and substitutions for improving stability. It should be noted that these substitutions can be combined with any other substitutions such as production variants, affinity variants, and/or additional stability variants. The numbering is in the context of Human IL18 mature form sequence as depicted in FIG. 1.

FIG. 47 depicts additional IL18 positions and substitutions for improving stability, specifically in the context of cysteine engineering. These substitutions remove existing cysteine residues or introduce new cysteine residues. It should be noted that these substitutions can be combined with any other substitutions such as production variants, affinity variants, and/or additional stability variants. The numbering is in the context of Human IL18 mature form sequence as depicted in FIG. 1.

FIG. 48 depicts binding to human IL18BP, cynomolgus IL18BP, human IL18R1, cynomolgus IL18R1, human IL18R1/IL18RAP complex, and cynomolgus IL18R1/IL18RAP complex by additional IL18 affinity variants (in the context of 4CS and S10C/N155C variants). The data show that K53D is the most important residue related to detuning IL18BP binding, and K53A and P57E are the next two most important.

FIG. 49 depicts activation of KG-1 cells (as indicated by induction of PD-L1 expression) by additional IL18 combination affinity variants (in the context of monovIL18-Fc fusions). Affinity variants were engineered in the context of IL18-4CS further including S10C/N155C stability variants.

FIGS. 50A-50E depict activation of KG-1 cells (as indicated by induction of PD-L1 expression) by additional TL18 combination affinity variants in the absence of IL18BP (solid lines) or in the presence of 100 ng/ml IL18BP (dotted lines). Affinity variants were engineered in the context of IL18-4CS further including S10C/N155C stability variants. Vertical line indicates 100 ng/mL IL18BP=5.56 nM.

FIG. 51 depicts EC50 of activation of KG-1 cells (as indicated by induction of PD-L1 expression) by additional TL18 combination affinity variants in the absence of IL18BP (solid lines) or in the presence of 100 ng/ml IL18BP (dotted lines). Affinity variants were engineered in the context of IL18-4CS further including S10C/N155C stability variants.

FIG. 52 depicts activation of KG-1 cells (as indicated by induction of PD-L1 expression) by additional IL18 combination affinity variants. New affinity variants were engineered in the context of IL18-4CS further including S10C/N155C stability variants.

FIGS. 53A-53L depict activation of KG-1 cells (as indicated by induction of PD-L1 expression) by additional IL18 combination affinity variants in the absence of IL18BP (solid lines) or in the presence of 1 μg/ml IL18BP (dotted lines). New affinity variants were engineered in the context of IL18-4CS further including S10C/N155C stability variants. Vertical line indicates 1 μg/mL IL18BP=55.56 nM.

FIG. 54 depicts EC50 of activation of KG-1 cells (as indicated by induction of PD-L1 expression) by additional IL18 combination affinity variants in the absence of IL18BP or in the presence of 1 μg/ml IL18BP. Affinity variants were engineered in the context of L18-4CS further including S10C/N155C stability variants.

FIG. 55 depicts activation of KG-1 cells (as indicated by induction of PD-L1 expression) by additional single substitution IL18 affinity variants. New affinity variants were engineered in the context of L18-4CS further including S10C/N155C stability variants.

FIG. 56 depicts activation of KG-1 cells (as indicated by induction of PD-L1 expression) by additional single substitution IL18 affinity variants. New affinity variants were engineered in the context of L18-4CS further including S10C/N155C stability variants.

FIG. 57 depicts activation of KG-1 cells (as indicated by induction of PD-L1 expression) by additional IL18 combination affinity variants. New affinity variants were engineered in the context of L18-4CS further including S10C/N155C stability variants.

FIG. 58 depicts activation of KG-1 cells (as indicated by induction of PD-L1 expression) by additional IL18 combination affinity variants. New affinity variants were engineered in the context of L18-4CS further including S10C/N155C stability variants.

FIG. 59 depicts activation of KG-1 cells (as indicated by induction of PD-L1 expression) by additional IL18 combination affinity variants. New affinity variants were engineered in the context of L18-4CS further including S10C/N155C stability variants.

FIGS. 60A-60K depict activation of KG-1 cells (as indicated by induction of PD-L1 expression) by additional IL18 combination affinity variants in the absence of IL18BP (solid lines) or in the presence of 1 μg/ml IL18BP (dotted lines). New affinity variants were engineered in the context of IL18-4CS further including S10C/N155C stability variants. Vertical line indicates 1 μg/mL IL18BP=55.56 nM.

FIG. 61 depicts activation of KG-1 cells (as indicated by induction of PD-L1 expression) by additional IL18 combination affinity variants. New affinity variants were engineered in the context of IL18-4CS further including S10C/N155C stability variants.

FIG. 62 depicts EC50 of activation of KG-1 cells (as indicated by induction of PD-L1 expression) by additional IL18 combination affinity variants and their fold reduction from WT* (*note: IL18 comprising 4CS and S10C/N155C).

FIG. 63 depicts activation of KG-1 cells (as indicated by induction of PD-L1 expression) by selected IL18 potency variants in the context of monovIL18-Fc (solid symbol) or IL18×Fab-Fc (open symbol). The data suggest that IL18× Fab-Fc fusions have lower Ymax (efficacy).

FIGS. 64A-64E depict change in body weight (as an indicator of GVHD) by A) Day 6, B) Day 9, C) Day 13, D) Day 16, and E) over time in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT).

FIGS. 65A-65B depict serum huIL18BP on A) Day 7 and B) Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show that the IL18 fusion proteins induced huIL18BP over time.

FIGS. 66A-66C depict expansion of A) human CD4 T cells, B) human CD8 T cell, and C) human CD16/CD56 NK cells by Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show T cell expansion generally correlated with IL18 potency (and was dose dependent).

FIG. 67 depicts activation of human CD16/CD56 NK cells (as indicated by NKG2D expression) by Day 7 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT).

FIGS. 68A-68B depict serum IFNγ on A) Day 7 and B) Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show that the TL18 fusion proteins induced IFNγ over time.

FIGS. 69A-69B depict serum GM-CSF on A) Day 7 and B) Day 14 in huPBMC-engrafted NSG mice dosed with PBS or potency reduced IL18 fusion proteins at 5.0 mg/kg or 0.5 mg/kg. Test articles included XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). The data show that the IL18 fusion proteins induced serum GM-CSF over time.

FIGS. 70A-70C depict serum concentration of IL18 fusion proteins over time in C57/B16 mice after dosing with 2 mg/kg XENP39804 (monovIL18-Fc with 4CS/E31Q variant), XENP40685 (monovIL18-Fc with 4CS/E31Q variant and further including S10C/N155C), or XENP40686 (I8× Fab-Fc with 4CS/E31Q variant and further including S10C/N155C). The data show improvement in serum levels upon introduction of the S10C/N155C disulfide variant into the E31Q base (i.e., XENP40685 vs. XENP39804). The IL18× Fab-Fc format did not appear to confer any further improvements (i.e., XENP40686 vs. XENP40685).

FIG. 71 depicts human PBMCs stimulated with 500 ng/mL plate-bound anti-CD3 (OKT3) for 48 h and then analyzed by flow cytometry. Gates were selected for naïve (CD28+CD95−), memory (CD28+CD95+), and effector (CD28-CD95mid) T cells. Central Memory (Tcm) and Stem-cell like memory (Tscm) T cells were further gated by CD45RA− or CD45RA+, respectively. Counts and IL18R1 expression were consistent between CD4 and CD8 populations. Data show that IL18R1 expression is biased towards NKs and memory T cell subsets.

FIG. 72 depicts the unfolding transition of various IL18 variants as determined by differential scanning fluorimetry.

FIG. 73 depicts the stabilization of IL18 that enabled improved solution behavior and facile purification. Analytical SEC and analytical IEX chromatograms of purified, stabilized IL18-Fc material are depicted.

FIGS. 74A-74C depict activation of KG-1 cells (as indicated by induction of PD-L1 expression) by A) XENP41756 (4CS_S10C/N155C), B) XENP42006 (4CS_E6Q/S10C/K53D/N111T/N155C), and C) XENP41762 (4CS_S10C/K53D/N155C) in the absence of IL18BP (solid circle) or in the presence of 10 μg/ml IL18BP (open). Vertical line indicates 10 μg/mL IL18BP=555 nM.

FIGS. 75A-75G depict change in body weight (as an indicator of GVHD) by A) Day 8, B) Day 12, C) Day 15, D) Day 19, E) Day 25, F) Day 28, and G) Day 33 in huPBMC-engrafted NSG mice dosed with PBS or indicate concentrations of XENP41770 (4CS_S10C/E31Q/D35N/N155C), XENP42006 (4CS_E6Q/S10C/K53D/N111T/N155C), and XENP41762 (4CS_S10C/K53D/N155C).

FIGS. 76A-76B depict expansion of A) human CD3 T cells and B) human NK cells on Day 14 in huPBMC-engrafted NSG mice dosed with PBS or indicate concentrations of XENP41770, XENP42006, and XENP41762.

FIG. 77 depicts serum IFNγ on Day 7 in huPBMC-engrafted NSG mice dosed with PBS or indicate concentrations of XENP41770, XENP42006, and XENP41762.

FIG. 78 depicts serum concentration of XENP41974 (4CS_S10C/N155C) (total IL18-Fc vs. active IL18-Fc) over time in cynomolgus monkeys. Total IL18-Fc includes IL18-Fc with and without bound IL18BP, and active IL18-Fc is unbound.

FIG. 79 depicts serum concentration of XENP42007 (4CS_E6Q/S10D/K53D/N111T) (total IL18-Fc vs. active IL18-Fc) over time in cynomolgus monkeys. Total IL18-Fc includes IL18-Fc with and without bound IL18BP, and active IL18-Fc is unbound.

FIGS. 80A-80R depict the amino acid sequences of IL18-Fc fusion of the disclosure.

FIG. 81 depicts secretion of IP10 by human PBMCs upon stimulation with IL18-Fc fusions XENP42141, XENP42143, and XENP42145.

FIGS. 82A-82F depict activation of mouse NK1.1 cells (as indicated by induction of intracellular IFNγ) by murine IL18-Fc fusion having murine IL18 variants.

FIGS. 83A-83D depict binding to murine IL18BP by murine IL18-Fc fusion having murine IL18 variants. The data show that incorporating L59K dramatically lowers affinity to IL18BP (XENP43092 in FIG. 83A and FIG. 83B), adding K52X to L59K abrogates IL18BP binding (FIG. 83C), and adding E30Q or D34N does not modulate IL18BP binding (FIG. 83D).

FIGS. 84A-84J show sensorgrams depicting binding by IL18-Fc fusions for IL18BP as determined by Octet. It should be noted that this experiment was intended to ascertain binding and not intended for accurate KD measurements, but it does show relative ranking of binding enabled by various IL18 variants.

FIGS. 85A-85J show sensorgrams depicting binding by IL18-Fc fusions for IL18R1×IL18RAP heterodimer as determined by Octet. It should be noted that this experiment was intended to ascertain binding and not intended for accurate KD measurements, but it does show relative ranking of binding enabled by various IL18 variants.

FIGS. 86A-86F depict A) change in tumor volume (as determined by caliper measurements), B) NK cell counts, C) CD8 T cell counts, D) CD8:Treg ratio, E) serum IFNγ concentration, and F) % Granzyme B⁺ CD8 T cells on Days 7 or 14 (after 1^{st} dose on Day 0) in MCF7-engrafted CD34+Hu-NSG mice dosed with XENP42006 (at 1 mg/kg or 3 mg/kg) alone or in combination with anti-PD1 mAb (3 mg/kg), anti-PD1 mAb alone, or PBS control.

FIG. 87 depicts additional IL18 positions and substitutions for IL18 binding affinity for IL18 receptors and/or IL18BP. It should be noted that these substitutions can be combined with any other substitutions such as production variants, additional affinity variants, and/or stability variants. The numbering is in the context of Human IL18 mature form sequence as depicted in FIG. 1.

FIGS. 88A-88E depict sequences for further variants which explored additional substitutions modulating IL18 binding affinity for IL18 receptors and/or IL18BP. While these sequences include the 4CS and S10C/N155C stability substitutions, each of these sequences may exclude these substitutions. Additionally, it should be noted that each of these variants may include additional substitutions such as production variants, affinity variants, and/or stability variants.

FIG. 89 depicts activation of KG-1 cells (as indicated by induction of PD-L1 expression) by additional TL18 variants including 4CS, S10C/N155C, K53T and additional substitution at K8 (in the IL18× Fab-Fc (silent Fv) format).

FIG. 90 depicts activation of KG-1 cells (as indicated by induction of PD-L1 expression) by additional TL18 variants including 4CS, S10C/N155C, K53T and additional substitution at 149 (in the IL18× Fab-Fc (silent Fv) format).

FIG. 91 depicts activation of KG-1 cells (as indicated by induction of PD-L1 expression) by additional TL18 variants including 4CS, S10C/N155C, K53T and additional substitution at M60 (in the IL18× Fab-Fc (silent Fv) format).

FIG. 92 depicts activation of KG-1 cells (as indicated by induction of PD-L1 expression) by additional TL18 variants including 4CS, S10C/N155C, K53T and additional substitution at Q103 (in the IL18× Fab-Fc (silent Fv) format).

FIG. 93 depicts activation of KG-1 cells (as indicated by induction of PD-L1 expression) by additional TL18 variants including 4CS, S10C/N155C, K53T and additional substitution at V153 (in the TL18× Fab-Fc (silent Fv) format).

FIG. 94 depicts activation of KG-1 cells (as indicated by induction of PD-L1 expression) by several preferred IL18 variants (in the IL18× Fab-Fc (silent Fv) format).

FIGS. 95A-95H depict activation of KG-1 cells (as indicated by induction of PD-L1 expression) by A) XENP44157_4CS_S10C/I49Q/K53T/N155C, B) XENP44161_4CS_S10C/K53T/M60K/N155C, C), XENP44165_4CS_S10C/K53T/Q103R/N155C, D) XENP44167_4CS_S10C/K53T/V153K/N155C, E) XENP44173_4CS_S10C/N155C/H6-12, F) XENP44174_4CS_S10C/K53T/N155C, G) XENP41431_4CS_E6Q/S10C/K53D/N155C, and H) XENP41076_4CS_S10C/N155C (IL18× Fab-Fc (silent Fv) format) in the absence of IL18BP (solid line) or in the presence of 5 μg/mL IL18BP (dotted line). Vertical line indicates 5 μg/ml IL18BP=270 nM.

FIG. 96 depicts activation of KG-1 cells (as indicated by induction of PD-L1 expression) by additional IL18 variants (in the IL18× Fab-Fc (silent Fv) format) in the absence of IL18BP (solid line) or in the presence of 5 μg/mL IL18BP (dotted line). Vertical line indicates 5 μg/ml IL18BP=270 nM.

FIG. 97 depicts activation of KG-1 cells (as indicated by induction of PD-L1 expression) by additional IL18 variants (in the IL18× Fab-Fc (silent Fv) format) in the absence of IL18BP (solid line) or in the presence of 5 μg/mL IL18BP (dotted line). Vertical line indicates 5 μg/ml IL18BP=270 nM.

FIG. 98 depicts activation of KG-1 cells (as indicated by induction of PD-L1 expression) by additional IL18 variants (in the IL18× Fab-Fc (silent Fv) format) in the absence of IL18BP (solid line) or in the presence of 5 μg/mL IL18BP (dotted line). Vertical line indicates 5 μg/ml IL18BP=270 nM.

FIG. 99 depicts activation of KG-1 cells (as indicated by induction of PD-L1 expression) by additional IL18 variants (in the IL18× Fab-Fc (silent Fv) format) in the absence of IL18BP (solid line) or in the presence of 5 μg/mL IL18BP (dotted line). Vertical line indicates 5 μg/ml IL18BP=270 nM.

FIG. 100 depicts EC50 activation of KG-1 cells (as indicated by induction of PD-L1 expression) by additional IL18 variants (in the IL18× Fab-Fc (silent Fv) format) in the absence of (as well as X fold reduction compared to “WT” XENP41076) or in the presence of 5 μg/mL IL18BP.

FIG. 101 depicts activation of KG-1 cells (as indicated by induction of PD-L1 expression) by additional IL18 variants (in the IL18× Fab-Fc (silent Fv) format).

FIG. 102 depicts activation of KG-1 cells (as indicated by induction of PD-L1 expression) by additional IL18 variants (in the IL18× Fab-Fc (silent Fv) format).

FIG. 103 depicts activation of KG-1 cells (as indicated by induction of PD-L1 expression) by additional IL18 variants (in the IL18× Fab-Fc (silent Fv) format).

FIG. 104 depicts EC50 activation of KG-1 cells (as indicated by induction of PD-L1 expression) by additional IL18 variants (in the IL18× Fab-Fc (silent Fv) format).

FIG. 105 depicts activation of KG-1 cells (as indicated by induction of PD-L1 expression) by additional IL18 variants (in the IL18× Fab-Fc (silent Fv) format) in the absence of IL18BP (solid line) or in the presence of 5 μg/mL IL18BP (dotted line). Vertical line indicates 5 μg/ml IL18BP=270 nM.

FIG. 106 depicts EC50 activation of KG-1 cells (as indicated by induction of PD-L1 expression) by additional IL18 variants (in the IL18× Fab-Fc (silent Fv) format) in the absence of (as well as X fold reduction compared to “WT” XENP41076) or in the presence of 5 μg/mL IL18BP.

FIG. 107 depicts sequences for a comparator DR-mIL18 molecule.

FIG. 108 depicts activation of mouse NK1.1 cells (as indicated by induction of intracellular IFNγ) by murine IL18-Fc fusion having murine IL18 variants.

FIG. 109 depicts change in tumor volume (as determined by caliper measurements) over time (after 1^{st} dose on Day 1) in CT26-engrafted BALB/c mice dosed with indicated concentrations of XENP42594 (“WT” mIL18), XENP44058 (a comparator DR-mIL18, sequences depicted in FIG. 107), XENP43773 (mIL18 with mIL18_3CS_3CS_A10C/M50G/K52V/E55R/L59K/N153C having 9-fold reduced potency in comparison to “WT” mIL18), XENP43774 (mIL18 with mIL18_3CS_3CS_A10C/M50G/K52D/E55R/L59K/N153C having 84-fold reduced potency in comparison to “WT” mIL18), XENP43772 (mIL18 with mIL18_3CS_3CS_A10C/D34N/M50G/K52V/E55R/L59K/N153C having 336-fold reduced potency in comparison to “WT” mIL18) or PBS control.

FIGS. 110A-110B depict activation of KG-1 cells (as indicated by induction of PD-L1 expression) by additional TL18 variants (in the TL18× Fab-Fc (silent Fv) format) A) in the absence of IL18BP or B) in the presence of 5 μg/mL IL18BP (dotted line). Vertical line indicates 5 μg/ml IL18BP=270 nM. (1:1 IL18:IL18BP).

FIG. 111 depicts activity of IL18-Fc fusion (as indicated by upregulation of intracellular IFNγ in NK, CD4+, and CD8+ cells).

FIGS. 112A-112B depict serum concentration of additional IL18-Fc fusions (total IL18-Fc vs. active IL18-Fc) over time in cynomolgus monkeys. A) depicts total IL18-Fc (IL18+IL18:IL18BP) and B) depicts active, non-inhibited (unbound) IL18-Fc. Active forms of engineered IL18-Fc fusions exhibit slow receptor-mediate clearance in NHP.

FIG. 113 depicts change in tumor volume (as determined by caliper measurements) over time (after 1^{st} dose on Day 1) in CT26-engrafted BALB/c mice dosed with indicated concentrations of XENP42594 (“WT” mTL18), XENP44058 (a comparator DR-mTL18, sequences depicted in FIG. 107), XENP43773 (mTL18 with mIL18_3CS_3CS_A10C/M50G/K52V/E55R/L59K/N153C having 9-fold reduced potency in comparison to “WT” mTL18), XENP43774 (mIL18 with mIL18_3CS_3CS_A10C/M50G/K52D/E55R/L59K/N153C having 84-fold reduced potency in comparison to “WT” mTL18), XENP43772 (mIL18 with mIL18_3CS_3CS_A10C/D34N/M50G/K52V/E55R/L59K/N153C having 336-fold reduced potency in comparison to “WT” mTL18), or PBS control.

FIG. 114 depicts total IL18-Fc concentration on Day 9 in serum of mice.

FIGS. 115A-115C depict correlation of gene expression in Day 9 tumor infiltrates after treatment with XENP42594 (“WT” mIL18) vs. A) 43772 (mIL18 with mIL18_3CS_A10C/D34N/M50G/K52V/E55R/L59K/N153C having 336-fold reduced potency in comparison to “WT” mIL18), B) XENP43774 (mIL18 with mIL18_3CS_A10C/M50G/K52D/E55R/L59K/N153C having 84-fold reduced potency in comparison to “WT” mIL18), or C) XENP43773 (mIL18 with mIL18_3CS_A10C/M50G/K52V/E55R/L59K/N153C having 9-fold reduced potency in comparison to “WT” mIL18). The data show that potency reduction does not impact gene expression pattern.

FIG. 116 depicts change in tumor volume (as determined by caliper measurements) over time in 10 naïve Balb/c mice or 15 surviving mice (previously engrafted with CT26 tumor cells and treated with mIL18-Fc fusions) engrafted with CT26 tumor cells. The data show that tumor-free mice from engineered mIL18-Fc fusions prevent tumor growth upon rechallenge.

FIGS. 117A-117B depict binding of surrogate mIL18 potency variants to (A) mIL18R1×RAP and (B) mIL18BP.

FIGS. 118A-118H depict illustrative formats of the IL18 triAbs described herein. FIG. 118A depicts the “1+1+1 Fab-scFv-Fc×IL18-Fc” format which comprises a first monomer comprising a variable heavy (VH) region covalently attached (optionally via a domain linker) to the N-terminus of an scFv covalently attached (optionally via a domain linker) to the N-terminus of a first heterodimeric Fc chain, a second monomer comprising an IL18 monomer covalently attached (optionally via a domain linker) to the N-terminus of a complementary second heterodimeric Fc chain, and a third monomer that is a corresponding light chain VL-CL (wherein the VL forms a binding domain with the VH). FIG. 118B depicts the “1+1+1 scFv-Fc×IL18-Fab-Fc” format which comprises a first monomer comprising an scFv covalently attached (optionally via a domain linker) to the N-terminus of a first heterodimeric Fc chain, a second monomer comprising an IL18 monomer covalently attached (optionally via a domain linker) to the N-terminus of a VH region covalently attached (optionally via a domain linker) to the N-terminus of a complementary second heterodimeric Fc chain, and a third monomer that is a corresponding light chain VL-CL (wherein the VL forms a binding domain with the VH). FIG. 118C depicts the “1+1+1 IL18-scFv-Fc× Fab-Fc” format which comprises a first monomer comprising an IL18 covalently attached (optionally via a domain linker) to the N-terminus of an scFv covalently attached (optionally via a domain linker) to the N-terminus of a first heterodimeric Fc chain, a second monomer comprising a VH region covalently attached (optionally via a domain linker) to the N-terminus of a complementary second heterodimeric Fc chain, and a third monomer that is a corresponding light chain VL-CL (wherein the VL forms a binding domain with the VH). FIG. 118D depicts the “1+1+1 Fab-Fc-scFv×IL18-Fc” format which comprises a first monomer comprising a VH region covalently attached (optionally via a domain linker) to the N-terminus of a first heterodimeric Fc chain covalently attached (optionally via a domain linker) to the N-terminus of an scFv, a second monomer comprising an TL18 monomer covalently attached (optionally via a domain linker) to the N-terminus of a complementary second heterodimeric Fc chain, and a third monomer that is a corresponding light chain VL-CL (wherein the VL forms a binding domain with the VH). FIG. 118E depicts the “2+1+1 Fab-scFv-Fc×IL18-Fab-Fc” format which comprises a first monomer comprising a first VH region covalently attached (optionally via a domain linker) to the N-terminus of an scFv covalently attached (optionally via a domain linker) to the N-terminus of a first heterodimeric Fc chain, a second monomer comprising an IL18 monomer covalently attached (optionally via a domain linker) to the N-terminus of a second VH region covalently attached (optionally via a domain linker) to the N-terminus of a complementary second heterodimeric Fc chain, and a third monomer that is a corresponding light chain VL-CL (wherein the VL form binding domains with the VHs). FIG. 118F depicts the “2+1+1 Fab-Fab-Fc×IL18-scFv-Fc” format which comprises a first monomer comprising a first VH region covalently attached (optionally via a domain linker) to the N-terminus of a second VH region covalently attached (optionally via a domain linker) to the N-terminus of a first heterodimeric Fc chain, a second monomer comprising an IL18 monomer covalently attached (optionally via a domain linker) to the N-terminus of an scFv covalently attached (optionally via a domain linker) to the N-terminus of a complementary second heterodimeric Fc chain. FIG. 118G depicts the “1+1+1 IL18-Fab (via VH)-scFv-Fc×empty-Fc” format which comprises a first monomer comprising a IL18 monomer covalently attached (optionally via a domain linker) to the N-terminus of a variable heavy (VH) region covalently attached (optionally via a domain linker) to the N-terminus of an scFv covalently attached (optionally via a domain linker) to the N-terminus of a first heterodimeric Fc chain, a second monomer comprising a second heterodimeric Fc chain, and a third monomer that is a corresponding light chain VL-CL (wherein the VL forms a binding domain with the VH). FIG. 118H depicts the “1+1+1 IL18-Fab (via VL)-scFv-Fc×empty-Fc” format which comprises a first monomer comprising a variable heavy (VH) region covalently attached (optionally via a domain linker) to the N-terminus of an scFv covalently attached (optionally via a domain linker) to the N-terminus of a first heterodimeric Fc chain, a second monomer comprising a second heterodimeric Fc chain, and a third monomer comprising an IL18 monomer covalently attached (optionally via a domain linker) to the N-terminus of a variable light (VL) region covalently attached to the N-terminus of a constant light domain (wherein the VL forms a binding domain with the VH).

FIG. 119 depicts the sequences for human, mouse, and cynomolgus CD3e. Such CD3e are useful for the development of cross-reactive CD3e antigen binding domains for ease of clinical development.

FIGS. 120A-120F depict sequences for exemplary anti-CD3 binding domains suitable for use in TAA×CD3×IL18 triAbs of the invention. The CDRs are underlined, the scFv linker is double underlined (in the sequences, the scFv linker is a positively charged scFv (GKPGS)4 linker (SEQ ID NO: 1574), although as will be appreciated by those in the art, this linker can be replaced by other linkers, including uncharged or negatively charged linkers, some of which are depicted in FIG. 5), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 121A-121B depict the sequences for human, mouse, and cynomolgus B7H3. Such B7H3 are useful for the development of cross-reactive B7H3 antigen binding domains for ease of clinical development.

FIGS. 122A-122K depict sequences for control molecules used herein.

FIGS. 123A-123M depict the sequences for illustrative IL18 triAbs of the 1+1+1 Fab-scFv-Fc×IL18-Fc format. CDRs are underlined and slashes (/) indicate the border(s) between variable domains, IL18 monomer, and other domains. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the 1+1+1 Fab-scFv-Fc×IL18-Fc sequences may utilize alternative backbones (including, but not limited, to those depicted in FIGS. 9A-9E) or alternative variable heavy and variable light domains. In addition, each sequence outlined herein can include or exclude the M428L/N434S (or M428L/N434S) variants in one or preferably both Fe domains, which results in longer half-life in serum.

FIGS. 124A-124F depict the sequences for illustrative IL18 triAbs of the 1+1+1 scFv-Fc×IL18-Fab-Fc format. CDRs are underlined and slashes (/) indicate the border(s) between variable domains, IL18 monomer, and other domains. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the 1+1+1 scFv-Fc×IL18-Fab-Fc sequences may utilize alternative backbones (including, but not limited, to those depicted in FIGS. 9A-9E) or alternative variable heavy and variable light domains. In addition, each sequence outlined herein can include or exclude the M428L/N434S (or 428L/434A) variants in one or preferably both Fc domains, which results in longer half-life in serum.

FIGS. 125A-125G depict the sequences for illustrative IL18 triAbs of the 1+1+1 IL18-scFv-Fc× Fab-Fc format. CDRs are underlined and slashes (/) indicate the border(s) between variable domains, IL18 monomer, and other domains. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the 1+1+1 IL18-scFv-Fc× Fab-Fc sequences may utilize alternative backbones (including, but not limited, to those depicted in FIGS. 9A-9E) or alternative variable heavy and variable light domains. In addition, each sequence outlined herein can include or exclude the M428L/N434S (or 428L/434A) variants in one or preferably both Fc domains, which results in longer half-life in serum.

FIGS. 126 and 127 depict the sequences for illustrative IL18 triAbs of the 1+1+1 Fab-Fc-scFv×IL18-Fc format. CDRs are underlined and slashes (/) indicate the border(s) between variable domains, IL18 monomer, and other domains. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the 1+1+1 Fab-Fc-scFv×IL18-Fc sequences may utilize alternative backbones (including, but not limited, to those depicted in FIGS. 9A-9E) or alternative variable heavy and variable light domains. In addition, each sequence outlined herein can include or exclude the M428L/N434S (or 428L/434A) variants in one or preferably both Fc domains, which results in longer half-life in serum.

FIGS. 128A-128C depict the sequences for illustrative IL18 triAbs of the 2+1+1 Fab-scFv-Fc×IL18-Fab-Fc format. CDRs are underlined and slashes (/) indicate the border(s) between variable domains, IL18 monomer, and other domains. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the 2+1+1 Fab-scFv-Fc×IL18-Fab-Fc sequences may utilize alternative backbones (including, but not limited, to those depicted in FIGS. 9A-9E) or alternative variable heavy and variable light domains. In addition, each sequence outlined herein can include or exclude the M428L/N434S (ore 428L/434A) variants in one or preferably both Fc domains, which results in longer half-life in serum.

FIG. 129 depicts the sequences for an illustrative IL18 triAbs of the 2+1+1 Fab-Fab-Fc×IL18-scFv-Fc format. CDRs are underlined and slashes (/) indicate the border(s) between variable domains, IL18 monomer, and other domains. It should be noted that IL18 sequences that are 90, 95, 98 and 99% identical (as defined herein), and/or contain from 1, 2, 3, 4, 5, 6, 7, 8, 9 or 10 additional amino acid substitutions and/or exclude amino acid substitutions, including production, affinity, and stability substitutions. Additionally, each of the 2+1+1 Fab-Fab-Fc×IL18-scFv-Fc sequences may utilize alternative backbones (including, but not limited, to those depicted in FIGS. 9A-E) or alternative variable heavy and variable light domains. In addition, each sequence outlined herein can include or exclude the M428L/N434S variants (ore 428L/434A) in one or preferably both Fc domains, which results in longer half-life in serum.

FIGS. 130A-130D depict induction of A) IFNγ, B) IL2, C) IL10, and D) TNFα secretion by B7H3×CD3×IL18 triAbs in 1+1+1 Fab-scFv-Fc×IL18-Fc format with either lower potency 4CS/S10C/D37N/K53D/N155C or higher potency 4CS/E6Q/S10C/K53D/N111T/N155C IL18 variants.

FIGS. 131A-131D depict induction of A) IFNγ, B) IL2, C) IL10, and D) TNFα secretion by B7H3×CD3×IL18 triAbs in 1+1+1 Fab-Fc×IL18-scFv-Fc format with either lower potency 4CS/S10C/D37N/K53D/N155C or higher potency 4CS/E6Q/S10C/K53D/N111T/N155C IL18 variants.

FIG. 132 depicts IFNγ secretion by B7H3×CD3×IL18 triAbs in 1+1+1 Fab-scFv-Fc×IL18-Fc, 1+1+1 Fab-Fc-scFv×IL18-Fc, and 1+1+1 Fab-Fc×IL18-scFv-Fc formats with either lower potency 4CS/S10C/D37N/K53D/N155C or higher potency 4CS/E6Q/S10C/K53D/N111T/N155C IL18 variants.

FIG. 133 depicts induction of IFNγ secretion by B7H3×CD3×IL18 triAbs in various formats and having 30 nM or 5 nM CD3 affinity and with either lower potency 4CS/S10C/D37N/K53D/N155C or higher potency 4CS/E6Q/S10C/K53D/N111T/N155C IL18 variants. The data show that regardless of format or IL18 potency, higher CD3 affinity increased potency.

FIGS. 134A-134B depict induction of A) Th1 (as indicated by percentage CCR5+CD4+ T cells) and B) TH2 (as indicated by percentage CCR4+CD4+ T cells) responses by B7H3×CD3×IL18 triAbs. The data show that while IL18-Fc induces Th1 polarization, the IL18 triAbs enhance Th2 response.

FIGS. 135A-135B depict induction of IFNγ secretion by B7H3×CD3×IL18 triAbs in the presence and absence of exogenous IL-12. The data show that IL18 triAbs enhance IFNγ secretion on isolated T cells in the presence of exogenous IL-12.

FIG. 136 depicts induction of IFNγ secretion by MSLN×CD3×IL18 triAbs in the 1+1+1 Fab-scFv-Fc×IL18-Fc, 1+1+1 scFv-Fc×IL18-Fab-Fc, and 2+1+1 Fab-scFv-Fc×IL18-Fab-Fc formats. The data show that each of IL18 triAbs induced IFNγ secretion, although the 1+1+1 scFv-Fc×IL18-Fab-Fc format was less potent.

FIG. 137 depicts induction of IFNγ secretion by MSLN×CD3×IL18 triAbs in the presence (solid symbol) or absence (open symbol) of cancer cells.

FIGS. 138A-138D depict A) induction of tumor (HepG2) cell killing (as indicated by % Zombie+ on target), B) T-cell activation (as indicated by CD107a expression on CD4 T cells), C) NK cell activation (as indicated by CD69 expression), and D) IL2 secretion by GPC3×CD3×IL18 triAbs.

FIGS. 139A-139C depict induction of tumor cell (Hep3B2) killing (as indicated by Luciferase) by GPC3×CD3×IL18 triAbs in A) 1+1+1 Fab-scFv-Fc×IL18-Fc, B) 2+1+1 Fab-Fab-Fc×IL18-scFv-Fc, and C) 2+1+1 Fab-scFv-Fc×IL18-Fab-Fc formats.

FIGS. 140A-140C depict group median change in tumor volume (as determined by caliper measurement; baseline corrected) over time (in days) in pp65-MCF7-gfp-engrafted CD34+Hu-NSG mice dosed with B7H3×CD3×IL18 triAbs XENP43917 or XENP44570 (2.1 mg/kg) or B7H3×CD3 XENP43918 (1.8 mg/kg) or PBS controls.

FIG. 141 depicts group median change in tumor volume (as determined by caliper measurements on mice with single tumors; baseline corrected) over time (in days) in huPBMC and HepG2-engrafted NSG-DKO mice dosed with GPC3×CD3×IL18 triAbs (XENP42989, XENP44506, XENP44569, or XENP43370) or GPC3×CD3 (alone or in combination with IL18-Fc) or PBS controls.

FIG. 142 depicts induction of IFNγ secretion by B7H3×CD3×IL18 triAbs in the 1+1+1 scFv-Fc×IL18-Fab-Fc and 1+1+1 IL18-scFv-Fc× Fab-Fc formats.

FIG. 143 depicts induction of IFNγ secretion by B7H3×CD3×IL18 triAbs in the 1+1+1 Fab-scFv-Fc×IL18-Fc format and having IL18 variants of differing potencies (as described in Example 11; 4CS_S10C/K53T/V153K/N155C>4CS_S10C/K53T/M60K/N155C>4CS_E6Q/S10C/K53D/N111T/N155C).

FIGS. 144A-144B depict induction of cancer cell (OVCAR8) kill by B7H3×CD3×IL18 triAbs in the 1+1+1 Fab-scFv-Fc×IL18-Fc format and having IL18 variants of differing potencies (as described in Example 11; 4CS_S10C/K53T/V153K/N155C>4CS_S10C/K53T/M60K/N155C>4CS_E6Q/S10C/K53D/N111T/N155C) in the presence of A) pre-stimulated PBMC and B) fresh PBMC.

FIGS. 145A-145D depict induction of A) IFNγ secretion, B) IL18Rα upregulation on CD8⁺ T cells, C) proliferation of CD8+IL18Rα⁺ T cells, and D) proliferation of CD8+IL18Rα⁻ T cells by B7H3×CD3×IL18 and RSV×CD3×IL18 triAbs in the 1+1+1 Fab-scFv-Fc×IL18-Fc format and having IL18 variants of differing potencies.

FIG. 146 depicts the variable heavy and variable light chain sequences for 6A1 and 3C4, exemplary humanized rat hybridoma-derived B7H3 binding domains.

FIG. 147 depicts the variable heavy and variable light chain sequences for 4F12 and 38E2, exemplary humanized rabbit hybridoma-derived B7H3 binding domains.

FIGS. 148A-148H depict the variable heavy and variable light chain sequences for 2E4A3.189, an exemplary phage-derived B7H3 binding domain, and additional sequences for affinity-optimized variable heavy domains from anti-B7H3 clone 2E4A3.189. It should be noted that the variable heavy domains can be paired with either 2E4A3.189_L1, as depicted in FIG. 148A.

FIGS. 149A-149R depict the variable heavy and variable light chain sequences for additional B7H3 binding domains which find use in the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 150A-150C depict anti-human EGFR antigen binding domains of use in the present invention.

FIGS. 151A-151B depict anti-human HER2 antigen binding domains of use in the present invention.

FIG. 152 depicts anti-human CD19 antigen binding domains of use in the present invention.

FIGS. 153A-153C depict anti-human CD20 antigen binding domains of use in the present invention.

FIGS. 154A-154B depict anti-CAIX and CD123 antigen binding domains of use in the present invention.

FIG. 155 depicts anti-human FLT3 antigen binding domains of use in the present invention.

FIGS. 156A-156Z and 156AA-156FF depict anti-human MSLN antigen binding domains of use in the present invention.

FIGS. 157A-157R depict anti-human Trop-2 antigen binding domains of use in the present invention.

FIGS. 158A-158C depict anti-human CEA antigen binding domains of use in the present invention.

FIG. 159 depicts anti-human CLDN18.2 antigen binding domains of use in the present invention.

FIGS. 160A-160C depict anti-human BCMA antigen binding domains of use in the present invention.

FIGS. 161A-161G depict anti-human PD-1 antigen binding domains of use in the present invention. Note that slashes (“/”) are placed between variable (VH and VL) and constant domains of the ABDs.

FIG. 162 depicts binding of GPC3×CD3×IL18 triAbs in the 1+1+1 Fab-scFv-Fc×IL18-Fc and 1+1+1 scFv-Fc×IL18-Fab-Fc formats (and empty or inactive IL18 variant controls) to GPC3+HepG2 cells. The data show that stacking IL18 on top of the GPC3 Fab decreases cell binding.

FIGS. 163A-163B depict induction of IFNγ secretion by A) purified T cells and B) human PBMCs following treatment with GPC3×CD3×IL18 triAbs in the 1+1+1 Fab-scFv-Fc×IL18-Fc and 1+1+1 scFv-Fc×IL18-Fab-Fc formats (and empty or inactive IL18 variant controls). The data show that an active IL18 arm potentiates CD3 dependent IFNγ secretion from T cells and PBMCs.

FIGS. 164A-164B depict induction of tumor cell kill by human PBMCs in the presence of A) GPC3×CD3×IL18 triAbs alone or B) triAbs in combination with IL12. The data show combining with IL12 potentiates CD3 mediated tumor cell killing.

FIGS. 165A-165B depict induction of IFNγ secretion by MSLN×CD3×IL18 triAbs in A) 1+1+1 scFv-Fc×IL18-Fab-Fc and B) 1+1+1 IL18-scFv-Fc× Fab-Fc formats, with IL18 of different potencies (4CS/S10C/K53T/V153K/N155C>4CS/S10C/K53T/M60K/N155C>4CS/E6Q/S10C/K53D/N111T/N155C).

FIGS. 166A-166C depict induction of IFNγ secretion by MSLN×CD3×IL18 triAbs in 1+1+1 Fab-scFv-Fc×IL18-Fc with A) 5 nM CD3 scFv, B) 30 nM CD3 scFv, and C) 70 nM CD3 scFv and IL18 of different potencies (4CS/S10C/K53T/V153K/N155C>4CS/S10C/K53T/M60K/N155C>4CS/E6Q/S10C/K53D/N111T/N155C).

FIGS. 167A-167B depict induction of IFNγ secretion by MSLN×CD3×IL18 triAbs in various formats at A) 1:1 and B) 1:10 effector (pre-stimulated T cell): target (OVCAR8).

### DETAILED DESCRIPTION

### I. Overview

An issue in the development of therapies using IL18 is that the activity of IL18 can be suppressed by the IL18 binding protein (IL18BP) which binds to IL18 and prevents it from binding to the IL18 receptor, which limits the utility of any IL18 therapies. Accordingly, the present invention provides IL18-Fc fusion proteins that generally comprise a variant human IL18 protein (as well as additional components as outlined herein). In general, the variant human IL18 protein is a variant of human IL18 (SEQ ID NO: 2), that includes amino acid modifications that reduce the binding of the variant IL18 to human IL18BP as described below. The IL18-Fc fusion proteins of the invention can take on a variety of different formats, as provided herein. In general, the constructs can comprise fusion proteins with just IL18 and Fc domains, fusion proteins with IL-18 and a tumor target antigen (TTA; sometimes referred to herein as “bispecific IL18 Fc fusion proteins”), or fusion proteins with IL-18, a TTA and a CD3 antigen binding domain (ABD; sometimes referred to herein as “trispecific fusion proteins”). Accordingly, provided herein are IL18-Fc fusion proteins that include an empty-Fc domain and an IL18 protein connected to another Fc domain such that the Fc domains form a heterodimer. Also provided herein are additional IL18-Fc fusion proteins such as IL18× Fab-Fc fusion proteins that include a first monomer containing an IL18 protein connected to a first Fc domain, a second monomer containing a variable heavy chain connected to a second Fc domain, and a variable light chain, such that the variable heavy and light chains form a Fab. In some embodiments, the Fab binds to a TTA. Also, provided herein are trispecific IL18-Fc fusion proteins that have three components: an IL18 protein (as more fully described herein, this is generally a variant human IL18 protein), an antigen binding domain (ABD) that binds to human CD3 (e.g. the extracellular domain of human CD3F) and an ABD that binds to a tumor target antigen (TTA), including, but not limited to, EGFR, Trop2, CD20, B7H3, FLT3, CD19, CD123, CD22, CD38, CEA, MSLN, BCMA, CAIX, CLDN18.2, HER2, PD-1, ANO1, and GPC3. Such IL18-Fc fusion proteins exhibit IL18 biological activity and long serum half-lives. Due to the long serum half-lives, the fusion proteins advantageously do not require high doses for use in treatments, thereby minimizing any potential systemic toxicity associated with increased IL18 levels. The IL18-Fc fusion proteins can be used for applications where increased IL18 activity is useful, for example, for increasing an immune response which can be useful for mounting an anti-cancer response in a subject in need thereof. Also described herein are various variant IL18 proteins with modifications to improve production (e.g., by improving yield and/or reduce heterogeneity), improve stability, reduce sink, and/or reduced affinity/potency.

### II. Definitions

In order that the application may be more completely understood, several definitions are set forth below. Such definitions are meant to encompass grammatical equivalents.

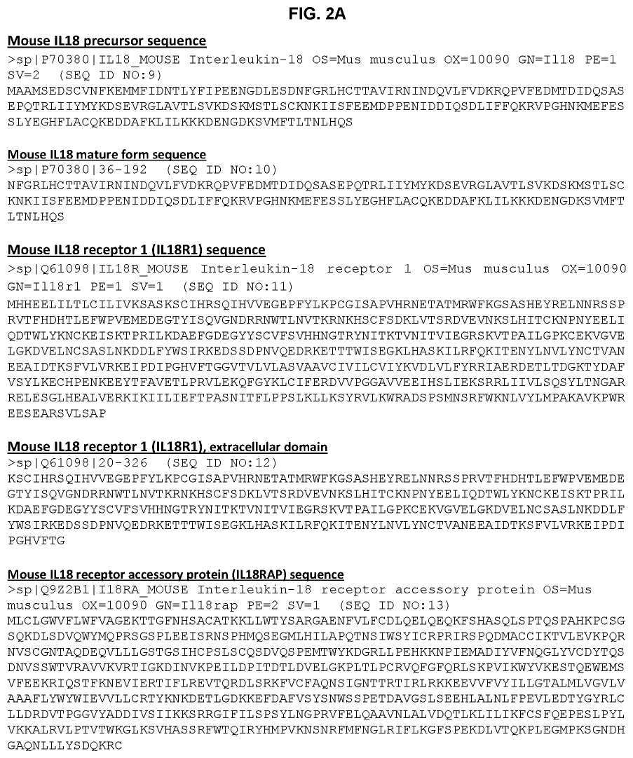

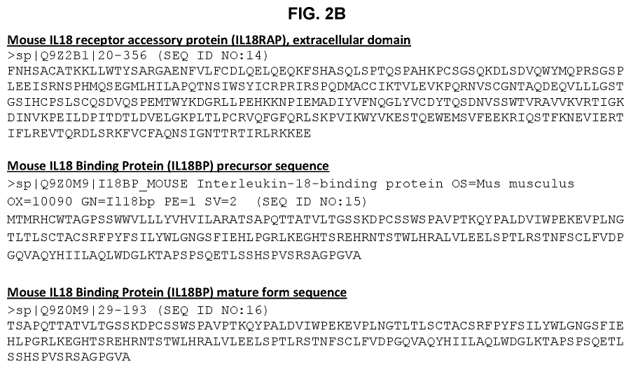

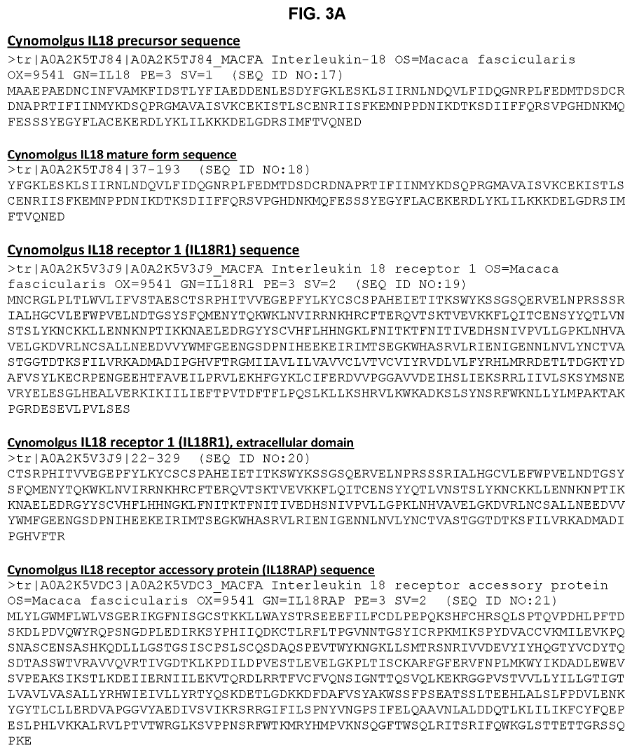

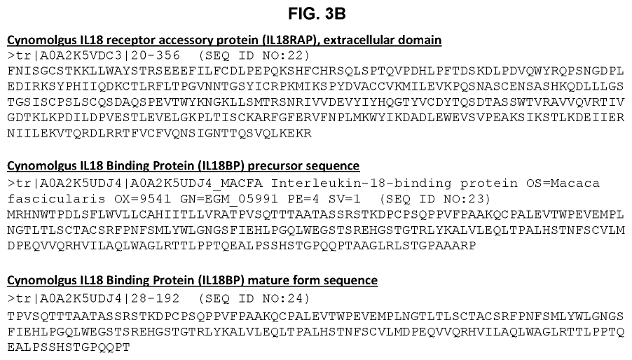

By “IL18,” “Interleukin-18,” and “IL-18” herein is meant a proinflammatory cytokine that binds to IL18 receptor (IL18R1). IL18 is capable of stimulating IFNγ production and regulating Th1 and Th2 responses. Sequences of various IL18 proteins, corresponding IL18 receptors, IL18 receptor accessory proteins (IL18RAPs), and IL18 binding proteins (IL18BPs) are shown in FIGS. 1-3. Sequences of exemplary wildtype human precursor and mature IL18, as well as the IL18 receptor subunits are included in FIGS. 1A-1B.

By “IL18BP,” “IL-18BP,” “IL18 binding protein.” or “IL-18 binding protein” herein is meant a protein that binds to IL18 and prevents IL18 from binding the IL18 receptor.

By “CD3” herein is meant a protein complex composed of 4 distinct protein chains including a CD3γ chain, a CD3δ chain, and two CD3ε chains. The CD3 antigen associates with a T-cell receptor (TCR) to form a CD3-TCR complex on T lymphocytes. The CD3 ABDs of the invention bind to the extracellular domain of human CD3ε.

By “ablation” herein is meant a decrease or removal of binding and/or activity. Thus, for example, “ablating FcγR binding” means the Fc region amino acid variant has less than 50% starting binding as compared to an Fc region not containing the specific variant, with more than 70-80-90-95-98% loss of binding being preferred, and in general, with the binding being below the level of detectable binding in a Biacore assay. Of particular use in the ablation of FcγR binding are those shown in FIG. 6. However, unless otherwise noted, the Fc monomers of the invention retain binding to the FcRn.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, some embodiments ablate ADCC activity entirely.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y or 272Y refers to a variant polypeptide, in this case an Fc variant, in which the glutamic acid at position 272 is replaced with tyrosine. For clarity, a protein which has been engineered to change the nucleic acid coding sequence but not to change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid residue or sequence at a particular position in a parent polypeptide sequence. For example, −233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid residue or sequence at a particular position in a parent polypeptide sequence. For example, E233−, E233 #, E233( ), E233_, or E233del designates a deletion of glutamic acid at position 233. Additionally, EDA233− or EDA233 #designates a deletion of the sequence GluAspAla that begins at position 233.

By “variant protein”, “protein variant”, or “variant” as used herein is meant a protein that differs from that of a parent protein by virtue of at least one amino acid modification (including amino acid substitutions, insertions and deletions). Protein variant may refer to the protein itself, a composition comprising the protein, the amino acid sequence that encodes it, or the DNA sequence that encodes it. Preferably, the protein variant has at least one amino acid modification compared to the parent protein, e.g., from about one to about seventy amino acid modifications, and preferably from about one to about five amino acid modifications compared to the parent. The modification can be an addition, deletion, or substitution. As described below, in some embodiments the parent protein, for example an Fc parent polypeptide, is a human wild type sequence, such as the Fc region from human IgG1, IgG2, IgG3 or IgG4. The protein variant sequence herein will preferably possess at least about 80% identity with a parent protein sequence, and most preferably at least about 90% identity, more preferably at least about 95-98-99% identity. “Variant,” as used herein can also refer to particular amino acid modifications (e.g., substitutions, deletions, insertions) in a variant protein (e.g., a variant Fc domain), for example, heterodimerization variants, ablation variants, FcKO variants, etc., as disclosed in Section III below.

As used herein, by “protein” is meant at least two covalently attached amino acids, which includes proteins, polypeptides, oligopeptides and peptides. When a biologically functional molecule comprises two or more proteins, each protein may be referred to as a “monomer” or as a “subunit; and the biologically functional molecule may be referred to as a “complex.”

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein with respect to an IgG domain is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” or “Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin (ß2-microglobulin) and the heavy chain is encoded by the FcRn gene. Unless otherwise noted herein, FcRn or an FcRn protein refers to the complex of FcRn heavy chain with ß2-microglobulin. A variety of Fc variants can be used to increase binding to the FcRn, and in some cases, to increase serum half-life. In general, unless otherwise noted, the Fc monomers of the invention retain binding to the FcRn (and, as noted below, can include amino acid variants to increase binding to the FcRn).

By “parent polypeptide” as used herein is meant a starting polypeptide that is subsequently modified to generate a variant. The parent polypeptide may be a naturally occurring polypeptide (i.e., a wildtype polypeptide), or a variant or engineered version of a naturally occurring polypeptide. Parent polypeptide may refer to the polypeptide itself, compositions that comprise the parent polypeptide, or the amino acid sequence that encodes it.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminus, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in certain embodiments, wherein the Fe domain is a human IgG1 Fe domain, the hinge includes a C220S amino acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU index as in Kabat. In some embodiments, as is more fully described below, amino acid modifications are made to the Fc region, for example to alter binding to one or more FcγR or to the FcRn.

As will be appreciated by those in the art, the exact numbering and placement of the heavy constant region domains can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

“Fc variant” or “variant Fc” as used herein is meant a protein comprising an amino acid modification in an Fc domain. The modification can be an addition, deletion, or substitution. The Fc variants of the present invention are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fe domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution. In many embodiments, the variant Fc is a variant of a human IgG1 Fc.

By “fusion protein” as used herein is meant covalent joining of at least two proteins or protein domains. Fusion proteins may comprise artificial sequences, e.g., a domain linker, an Fc domain (e.g., a variant Fc domain), an IL18 (e.g., a variant IL18), etc., as described herein. By “Fc fusion protein” herein is meant a protein comprising an Fc region, generally linked (optionally through a domain linker, as described herein) to one or more different protein domains. Accordingly, an “IL18-Fc fusion” includes an Fc domain linked (optionally through a domain linker) to an IL18, as described herein. In some instances, two Fc fusion proteins can form a homodimeric Fc fusion protein or a heterodimeric Fc fusion protein. In some embodiments, one monomer of the heterodimeric IL18-Fc fusion protein includes an Fc domain alone (e.g., an “empty Fc domain”) and the other monomer is an Fc fusion, comprising an IL18, as outlined herein. In other embodiments, both the first and second monomers are Fc fusion proteins that include an Fc domain and an IL18.

By “position” as used herein is meant a location in the sequence of a protein. Positions may be numbered sequentially, or according to an established format, for example the EU index for numbering of antibody domains (e.g., a CH1, CH2, CH3 or hinge domain).

By “strandedness” in the context of the monomers of the heterodimeric proteins of the invention herein is meant that, similar to the two strands of DNA that “match”, heterodimerization variants are incorporated into each monomer so as to preserve, create, and/or enhance the ability to “match” to form heterodimers. For example, if some pI variants are engineered into monomer A (e.g., making the pI higher), then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “wild type,” “wildtype” or “WT” herein is meant an amino acid sequence or a nucleotide sequence that is found in nature, including allelic variations. A WT protein has an amino acid sequence or a nucleotide sequence that has not been intentionally modified.

The IL18-Fc fusion proteins and variant IL18s provided herein are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated protein,” refers to a protein which is substantially free of other proteins from a cell culture such as host cell proteins. “Recombinant” means the proteins are generated using recombinant nucleic acid techniques in exogeneous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Publ. App. No. 20160244525, hereby incorporated by reference.

The degree of identity between an amino acid sequence provided herein (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

By “fused” or “covalently linked” is herein meant that the components (e.g., an IL18 and an Fc domain) are linked by peptide bonds, either directly or indirectly via domain linkers, outlined herein.

The strength, or affinity, of specific binding can be expressed in terms of dissociation constant (KD) of the interaction, wherein a smaller KD represents greater affinity, and a larger KD represents lower affinity. Binding properties can be determined by methods well known in the art such as bio-layer interferometry and surface plasmon resonance-based methods. One such method entails measuring the rates of antigen-binding site/antigen or receptor/ligand complex association and dissociation, wherein rates depend on the concentration of the complex partners, the affinity of the interaction, and geometric parameters that equally influence the rate in both directions. Thus, both the association rate (ka) and the dissociation rate (kd) can be determined, and the ratio of kd/ka is equal to the dissociation constant KD (See Nature 361:186-187 (1993) and Davies et al. (1990) Annual Rev Biochem 59:439-473).

Specific binding for a particular molecule or an epitope can be exhibited, for example, by a molecule (e.g., IL18) having a KD for its binding partner (e.g., IL18 receptor) of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater. Typically, an antigen binding molecule that specifically binds an antigen will have a KD that is 20-, 50-, 100-, 500-, 1000-, 5,000-, 10,000- or more times greater for a control molecule relative to the antigen or epitope.

### III. IL18 Fc Fusion Proteins

In some aspects, provided herein are IL18-Fc fusion proteins that include a first monomer that includes a IL18 protein (e.g., wildtype IL18 or a variant thereof) and a first Fc domain comprising pI variants as described below and a second monomer that includes a second Fc domain. The IL18-Fc fusion proteins are based on the self-assembling nature of the two Fc domains on each monomer leading to a IL18-Fc fusion proteins. Heterodimeric IL18-Fc fusion are made by altering the amino acid sequence of each monomer as more fully discussed below.

In one aspect, the IL18-Fc fusion protein is a heterodimeric Fc fusion protein. Such heterodimeric IL18-Fc fusion protein include a first monomer and a second monomer, each having an Fc domain with different amino acid sequences (e.g., a monovalent IL18-Fc fusion protein). As will be appreciated, discussion herein of components of the IL18-Fc fusion proteins encompassed by the present disclosure is applicable to both homodimeric and heterodimeric Fc fusion proteins as appropriate, unless otherwise specified.

In some embodiments, the IL18-Fc fusion protein is a monovalent IL18 fusion (i.e., includes only one IL18). In such embodiments, the first monomer includes an Fc domain and an IL18 and the second monomer includes an Fc domain alone (i.e., no IL18, an “empty Fc domain,”).

The Fc domains can be derived from IgG Fc domains, e.g., IgG1, IgG2, IgG3 or IgG4 Fc domains, with IgG1 Fc domains finding particular use in the invention. As described herein, IgG1 Fc domains may be used, often, but not always in conjunction with ablation variants to ablate effector function. Similarly, when low effector function is desired, IgG4 Fc domains may be used.

For any of the dimeric IL18-Fc fusion proteins described herein, the carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDRs and the framework and made a list thereof (see SEQUENCES OF IMMUNOLOGICAL INTEREST, 5th edition, NIH publication, No. 91-3242, E. A. Kabat et al., entirely incorporated by reference). Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present IL18 Fc fusion proteins are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

By “hinge” or “hinge region” or “antibody hinge region” or “immunoglobulin hinge region” herein is meant the flexible polypeptide comprising the amino acids between the first and second heavy chain constant domains of an antibody. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index as in Kabat. In some embodiments, for example in the context of an Fc region, the hinge (full length or a fragment of the hinge) is included, generally referring to positions 216-230. As noted herein, pI variants can be made in the hinge region as well.

In some embodiments of the IL18 fusion proteins described herein, each of the first and second monomers include an Fc domain that has the formula hinge-CH2-CH3. In some embodiments of the IL18 fusion proteins described herein, each of the first and second monomers include an Fc domain that has the formula CH2-CH3.

In some embodiments, provided herein is an IL18-Fc fusion protein which is also referred to as “an IL18× Fab-Fc fusion protein”. Such an IL18× Fab-Fc fusion protein includes a first monomer containing a variable heavy chain and an Fc domain; a second monomer containing and an IL18 protein (e.g., a wildtype IL18 protein or a variant thereof) and an Fc domain comprising pI variants as described below, and a third monomer containing a variable light chain such that the variable heavy and light chains form a Fab. In certain embodiments, the IL18 is directly connected to the Fc domain. In some embodiments, the C-terminus of the IL18 is directly connected to the N-terminus of the Fc domain. In some embodiments described herein, the IL18× Fab-Fc fusion protein also includes a second monomer that includes a second Fc domain and a Fab. In certain embodiments, the Fab is directly connected to the second Fc domain. In certain embodiments, the Fab is connected to the second Fc domain via a linker, such as but not limited to a domain linker. In some embodiments, the heavy chain of the Fab is directly connected to the second Fc domain. In some embodiments, the heavy chain of the Fab is connected to the second Fc domain via a linker, such as but not limited to a domain linker. In some embodiments, the C-terminus of the heavy chain of the Fab is directly connected to the N-terminus of the second Fc domain. In some embodiments, the light chain of the Fab is not directed connected to the second monomer. In certain embodiments, the light chain of the Fab is not directed connected to the first monomer.

In certain embodiments, the IL18 is connected to the Fc domain by a linker. In certain embodiments, the linker is a domain linker. Useful domain linkers include, but are not limited to, those disclosed in FIG. 8. While any suitable linker can be used, many embodiments utilize a glycine-serine polymer, including for example (GS)n, (GSGGS)n [SEQ ID NO:778], (GGGGS)n [SEQ ID NO:777], and (GGGS)n [SEQ ID NO:779], where n is an integer of at least one (and generally from 0 to 1 to 2 to 3 to 4 to 5) as well as any peptide sequence that allows for recombinant attachment of the two domains with sufficient length and flexibility to allow each domain to retain its biological function. In some cases, and with attention being paid to “strandedness”, as outlined below, the linker is a charged domain linker.

In certain embodiments, the IL18 fusion protein includes a first monomer, wherein an IL18 is connected to the Fc domain by a domain linker. In some embodiments, the C-terminus of the IL18 is connected to the N-terminus of the Fc domain by a domain linker.

### IV. Interleukin 18 Variants

The IL18-Fc fusion proteins provided herein include an IL18 protein. In some embodiments, the IL18-Fc fusion protein is a monovalent IL18-Fc fusion protein that includes one IL18. The IL18s that can be used with the IL18-Fc fusion proteins provided herein include wildtype IL18 (see FIGS. 1-3), functional fragments of such IL18s and variants that include 1, 2, 3, 4, 5, 6, 7, 8, 9, 10 amino acid modifications as compared to wildtype IL18 (e.g., wildtype human IL18 and wildtype human IL18, mature form). In some embodiments, the IL18 fusion protein or construct provided herein includes a wildtype human IL18 protein or a variant human IL18 protein such as any one of those described herein.

In some embodiments, the IL18 is a variant human IL18 that is at least 90, 91, 92, 93, 94, 95, 96, 97, 98, 99% identical to human IL18. In some embodiments, the IL18 is a variant human IL18 that is at least 90, 91, 92, 93, 94, 95, 96, 97, 98, 99% identical to human IL18, mature form. In particular embodiments, the IL18 includes 1, 2, 3, 4, 5, 6, 7, 8, 9, 10, 11, 12, 13, 14, 15, 16, 17, 18, 19 or 20 modifications as compared to wildtype human IL18. In particular embodiments, the IL18 includes 1, 2, 3, 4, 5, 6, 7, 8, 9, 10, 11, 12, 13, 14, 15, 16, 17, 18, 19 or 20 modifications as compared to wildtype human IL18.

Numbering of such IL18 modifications described herein are based on the human IL18 mature form sequence in FIG. 1, wherein the first amino acid of the sequence (“Y”) is amino acid position 1. Provided herein are compositions that include such a variant TL18 having one or more amino acid substitutions that reduce heterogeneity that may affect IL18-Fc fusion protein production and/or activity.

In certain embodiments, the IL18 variant includes one or more modifications to improve production. Such modifications are intended to improve yield and/or decrease molecular heterogeneity. Residues which may be modified to improve production include E31, C38, C68, C76, C127, D157. In some embodiments, the production variants include one or more amino acid substitutions selected from E31Q, C38S, C68S, C76S, C127S, D157del, D157S, and D157A.

In certain embodiments, the IL18 variant includes one or more modifications that are affinity variants which modulate binding affinity for IL18 receptors (such as IL18R1, IL18RAP, or the IL18R1:IL18RAP complex) and/or IL18BP. In some embodiments, the IL18 variant possesses one or more modifications that modulate IL18 potency and/or IL18 sink. Such modifications are believed to decrease the TL18BP sink and extend the half-life of the subject IL18-Fc fusion protein. Residues which may be modified to modulate binding affinity for IL18BP, IL18R1, IL18RAP, and/or the IL18R1:IL18RAP complex include Y1, E6, K8, D17, E31, D35, S36, D37, D40, N41, 149, M51, K53, S55, Q56, P57, M60, Q103, H109, D110, N111, and D132, and V153. In some embodiments, the affinity variants include one or more amino acid substitutions selected from Y1F, Y1H, E6A, E6Q, K8Y, K8Q, K8E, K8R, K8D, K8N, K8S, K8T, D17N, E31Q, D35N, D35E, S36D, S36N, D37N, D40N, N41Q, I49D, I49E, I49N, I49Q, I49Y, I49F, M51K, M51Q, M51I, M51R, M51L, M51H, M51F, M51Y, K53A, K53S, K53G, K53T, K53I, K53L, K53N, K53D, K53R, K53H, K53M, K53E, K53Q, K53V, K53Y, K53F, S55N, S55Q, S55D, S55E, S55T, Q56L, Q56I, P57A, P57E, P57Q, P57D, P57Y, P57N, M60L, M60I, M60K, M60Y, M60F, M60R, Q103E, Q103Y, Q103E, Q103K, Q103R, H109W, H109Y, D110N, D110Q, D110R, N111Q, N111S, N111T, N111E, D132Q, D132E, V153E, V153K, V153R, V153Y, V153Q, V153N, and V153D. In some embodiments, the amino acid substitution can include E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, or E6Q/M51I/K53T/V153K. In certain embodiments, the IL18 variant includes one or more modifications that modulate binding to IL18R including K53V. In certain other embodiments, the TL18 variant includes one or more modification that modulate binding to IL18BP including M60K.

In certain embodiments, the IL18 variant includes one or more modifications to improve stability. In some embodiments, the IL18 variant possesses one or more modifications that improve stability in the context of cysteine engineering (such as by removing unpaired cysteines and/or introducing new disulfide bridges). Residues which may be modified to improve stability include S7, S10, V11, N14, L15, Q18, D23, R27, P28, L29, E31, M33, T34, C38, R39, R44, I46, I49, 550, K53, D54, Q56, P57, M60, A61, V62, T63, S65, K67, C68, E69, 171, C76, E77, 180, 181, N87, P88, D90, K93, T95, K96, S97, Q103, N111, M113, 5119, A126, C127, L136, L138, K139, E141, L144, D146, R147, 1149, M150, N155, E156, and D157. In some embodiments, the stability variants include one or more amino acid substitutions selected from S7C, S7P, S10C, V11I, N14C, N14W, L15C, Q18L, D23N, D23S, R27Q, P28C, L29V, E31Q, M33C, T34P, C38Q, C38R, C38E, C38L, C38I, C38V, C38K, C38D, C38S, R39T, R39S, R44Q, I46V, I49C, S50C, S50Y, K53N, D54C, Q56L, P57T, P57V, P57A, P57E, M60L, A61C, V62C, T63C, S65C, K67Q, C68I, C68F, C68Y, C68D, C68N, C68E, C68Q, C68K, C68S, E69K, 171M, C76E, C76K, C76S, E77K, 180T, 181V, 181L, N87S, P88C, D90E, K93D, K93N, T95E, K96G, K96Q, S97N, Q103C, Q103L, Q103I, N111D, M113I, S119L, A126C, C127W, C127Y, C127F, C127D, C127E, C127K, C127S, L136C, L138C, K139C, E141K, E141Q, L144N, D146Y, D146L, D146F, R147C, R147K, 1149V, M150F, M150T, N155C, E156Q, and D157N. In some embodiments, the amino acid substitution can include Q56L/P57T, K93D/T95E, K93N/T95E, E156Q/D157N, D23N/R27Q, Q56L/T95E, K96Q/S119L, E141K/1149V, E141Q/1149V, S7P/S50Y, 180T/181L, P57A/S119L, P57A/180T/181L/S119L, P57A/K93D/T95E/S119L, 180T/S119L, 180T/181L/K93D/T95E, P57A/180T/181L/K93D/T95E/S119L, P57A/180T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, or C38R/C127W.

In some embodiments, one or more residues of IL18 selected from Y1, E6, S7, K8, 510, V11, N14, L15, D17, Q18, D23, R27, P28, L29, E31, M33, T34, D35, 536, D37, C38, R39, D40, N41, R44, 146, 149, 550, M51, K53, D54, S55, Q56, P57, M60, A61, V62, T63, S65, K67, C68, E69, 171, C76, E77, 180, 181, N87, P88, D90, K93, T95, K96, S97, Q103, H109, D110, N111, M113, 5119, A126, C127, D132, L136, L138, K139, E141, L144, D146, R147, 1149, M150, V153, N155, E156, and D157 are modified. In some embodiments, the IL18 variant described herein includes one or more amino acid substitutions selected from Y1F, Y1H, E6A, E6Q, S7C, S7P, K8E, K8Q, K8Y, K8R, K8D, K8N, K8S, K8T, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, I49D, I49E, I49N, I49Q, I49Y, I49F, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, M60R, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, Q103Y, Q103E, Q103K, Q103R, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, 1149V, M150F, M150T, V153E, V153K, V153R, V153Y, V153Q, V153N, V153D, N155C, E156Q, D157A, D157S, D157N, and D157del. In some embodiments, the IL18 variant includes a 4CS substitution (C38S/C68S/C76S/C127S substitutions) and one or more of the additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C, M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111 Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

Additional IL18 positions suitable for engineering substitutions, specific substitutions, and specific variants are known in the art which may find use in the IL18 variants and IL18-Fc fusions of the invention. Such positions, substitutions and variants are, for example, described in US 2021/0015891, US 2019/0070262, WO 2022/094473 and Zhou et al. 2020 (e.g., Y1, L5, K8, D17, E31, T34, D35, S36, D37, C38, D40, N41, M51, K53, S55, Q56, P57, G59, M60, C68, E77, Q103, S105, H109, D110, N111, M113, R131, V153, and N155 as in Y1R, Y1H, YlD, Y1L, Y1F, L5H, L51, L5Y, L5F, K8Q, K8R, D17G, D17R, D17H, D17A, E31K, E31A, E31T, E31G, E31R, T34E, T34K, T34A, D35A, D35S, D35Y, S36N, S36K, S36R, D37P, D37R, D37V, D37A, D37L, D37H, C38S, D40A, D40S, D40Y, N41R, N41S, N41K, M51T, M51K, M51D, M51N, M51E, M51R, M51F, M51I, M51L, K53R, K53G, K53S, K53T, S55K, S55R, Q56E, Q56A, Q56R, Q56V, Q56G, Q56K, Q56L, Q56H, P57L, P57G, P57A, P57K, G59A, G59T, M60K, M60Q, M60R, M60L, M60I, M60F, C68D, E77D, Q103E, Q103K, Q103P, Q103A, Q103R, Q103I, Q103L, S105D, S105N, S105R, S105K, S105A, H109A, H109P, H109D, D110K, D110H, D110N, D110Q, D110E, D110S, D110G, N111H, N111Y, N111D, N111R, N111S, N111G, M113V, M113R, M113T, M113K, M113F, M113I, M113L, R131S, V153I, V153T, V153A, N155K, N155H, M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110 S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53 S/Q56R/P57A/M60L/Q103A/S105D/D110S/N111G/M113R, Y1H/D17G/E31K/D35A/D40A/M51F/Q103I/H109A/M113F, Y1D/E31A/S36N/D37P/D40S/M51F/M60I/Q103L/H109P, Y1H/D17G/E31A/T34E/D35A/D37P/M51F/M60L/Q103L, Y1L/D17G/E31A/D35S/D37P/D40S/N41R/M51F/M60F/Q103I/M113I, Y1H/E31T/T34E/D35S/S36K/D37P/N41S/M60I/Q103I/M113F, D17R/E31T/D35Y/S36K/M51F/M60F/Q103I/H109A/M113I, Y1H/D17G/E31G/T34E/D35A/D37P/M51F/Q103I/H109A/M113L, Y1H/D17G/E31T/D35S/N41K/M51F/M60L/Q103L/H109P/M113F, Y1H/D17G/E31A/T34E/D35S/S36N/D37R/N41S/M60F/Q103L/M113I, D17G/E31T/D35A/D37P/M51F/M60L/Q103L/M113I/D132S, Y1D/E31A/T34K/D35S/S36N/D37V/D40A/Q103I/H109P/M113L, Y1H/D17G/E31G/T34E/D35A/D37P/D40A/N41K/M51I/M60F/Q103L/M113F/D132S, Y1H/D17G/E31A/T34A/D35S/S36N/D37R/D40A/M60F/Q103I/H109P/M113F, E31A/T34K/D35A/S36K/D37A/D40S/M51F/M60F/Q103L/H109A/M113I, Y1H/D17H/E31T/T34A/S36N/D37A/M51F/M60L/Q103I/H109D, D17G/E31T/T34K/D35S/S36N/D37L/D40S/M51F/M60I/Q103I/H109D/M113I, Y1H/D17G/E31R/T34A/D35A/D37H/N41S/M51I/M60F/Q103L/M113I, E31A/T34A/D35S/S36N/D37P/D40Y/N41K/M60L/Q103L/H109A/M113L, Y1H/D17G/E31T/T34A/D35S/S36N/D37H/D40Y/M60L/Q103L/H109A/M113I, Y1H/D17G/E31A/S36N/D37P/D40S/N41R/M51F/Q103I/M113F, D17G/E31T/D35A/D37H/D40S/N41S/M51L/Q103L/M113F, D17G/E31T/T34E/D35Y/S36R/D37L/D40A/M51F/Q56H/M60L/Q103L/H109D/M113L, D17G/E31T/T34E/D35S/D37L/M51L/Q103L/H109A/M113F/D132S, D17G/E31K/T34E/D35A/S36N/D40S/N41S/M51I/M60L/Q103I/H109A, Y1D/E31A/T34K/D35A/D37A/D40A/M60L/Q103L/M113F, D17G/E31G/D35A/S36K/D37H/D40A/N41R/M51F/Q103I/H109A/M113F, Y1F/L5F/D17G/E31T/T34E/D35S/S36K/N41R/M51F/M60L/Q103L/M113L, Y1F/D17G/E31A/T34A/D35S/S36N/D37H/D40A/M51L/M60L/Q103I, D17G/E31A/D35S/S36K/D37R/M51L/M60F/Q103I/M113I, Y1L/L5H/D17G/E31G/T34E/D35S/D37P/D40Y/M51F/M60L/Q103I, D17A/E31T/D35A/D37P/D40S/N41S/M51L/M60L/Q103L/H109P/M113I, D17G/E31A/Q103L, D17G/E31A/D35S/M51F/Q103L, D17G/E31A/T34E/D35S/M51F/M60L/Q103L, M51K/K53S/Q56L/D110N/N111R, M51K/K53S/Q56L/P57A/M60L/D110N/N111R, M51K/K53S/Q56L/P57A/M60L/S105D/D110N/N111R, C38S/M51K/K53S/Q56L/P57A/M60L/C68D/S105D/D110S/N111R, M51G/K53A/Q56R/M60K, C38S/C68S, C38S/C68G, C38S/C68A, C38S/C68V, C38S/C68D, C38S/C68E, C38S/C68N, and Y1H/M51A/K53G/Q56R/P57A/M60K); WO2003057821 (S10T, I12V, T45S, F47Y, Y52F, I64V, F101Y, L5V, L20V, L20I, F21Y, I22V, V66I, S72T, S148F, K4E, E61, K8D, R13I, L15R, D17K, R27K, F30A, D35K, D37F, C38E, R39A, D40W, M51E, K53G, Q56I, R58A, V62K, D94K, T95F, R104L, G108I, N111K, K129F, R131D, D132L, L133E, F134A, M150T, and F151S); U.S. Pat. No. 6,800,479 (D35E and D40E); U.S. Pat. Nos. 7,524,488, 7,875,709, WO 2002/101049, and Kim et al. 2001 (e.g., E42, I85, K89, M96, D130, K132, P143, M149, and L189 as in E42A, K89A, and E42A/K89A); and Saetang et al. 2016 (e.g., E6K, M33Q, M60Q, and T63A). In some embodiments, one or more of the foregoing substitutions and variants may be used in combination with any of the other IL-18 substitutions and variants described herein (e.g. those that exhibit reduced binding affinity to the IL18 receptor 1 (IL18R1), IL18 receptor accessory protein (IL18RAP), IL18R1:L18RAP complex and/or the IL18 binding protein (IL18BP), compared to wildtype human IL18; those that exhibit reduced heterogeneity; those that exhibit improved production yield; those that exhibit modulated potency; those that exhibit improved stability, and/or those that exhibit reduced IL18BP sink) to engineer IL18 variants of the invention. In some embodiments, each of these substitutions and variants may be used alone or in combination in the IL18-Fc fusion proteins of the invention. In some embodiments, each of these substitutions and variants may be used in combination with other IL-18 substitutions and variants described herein in the IL18-Fc fusion proteins of the invention. In some embodiments, the IL18 variants or IL18-Fc fusions include alternative substitutions at any of the foregoing positions.

In some embodiments, the IL18 variant of the IL18-Fc fusion protein includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 41A-41C, 42A-42D, 43A-43B, 44A-44C, and 88A-88E. In certain embodiments, the IL18 variant includes an amino acid sequence set forth in SEQ ID NOS:84-238, and 799-949.

### V. Interleukin 18-Fusion Variants

In some embodiments, the IL18-Fc fusion proteins described include one or more modifications (including an amino acid addition, deletion and substitution) to reduce binding to IL18BP. In some embodiments, the IL18-Fc fusion proteins include one or more modifications to reduce binding to IL18 receptors. In some embodiments, the IL18-Fc fusion proteins include one or more modifications to improve stability of the fusion proteins compared to wildtype IL18. In some embodiments, the IL18-Fc fusion proteins include one or more modifications for improving the production (such as, but not limited to, reducing heterogeneity of such proteins during manufacturing) of such fusion proteins. In some embodiments, the IL18-Fc fusion proteins include one or more modifications to remove free cysteines.

In certain embodiments, the IL18 includes one or more modifications to reduce heterogeneity that may affect IL18-Fc fusion protein production and/or activity. In some embodiments, such IL18 variants include one or more modifications to remove one or more free cysteines. In some embodiments, a cysteine (C) of a wildtype IL18 is substituted with a serine (S). Exemplary residues that may be modified to reduce heterogeneity and improve production include one or more amino acid residues selected from the group including: C38, C68, C76 and C127.

Particular modifications to reduce heterogeneity include amino acid substitutions C38S, C68S, C76S, C127S, C38S/C68S, C38S/C76S, C38S/C127S, C68S/C76S, C68S/C127S, C76S/C127S, C38S/C68S/C76S, C38S/C68S/C127S, C38S/C76S/C127S, C68S/C76S/C127S, C38S/C68S/C76S/C127S, (also referred to as the “4CS” substitutions) and combinations thereof. In some embodiments, the amino acid substitutions C38S/C68S/C76S/C127S of IL18 is herein referred to as the “4CS” substitutions.

In certain embodiments, the IL18-Fc fusion proteins include one or more modifications to improve production of such fusion proteins. For instance, one or more of the modifications is directed to reduce the potential impact of post-translational modification related liabilities. Residues which may be modified include removing the C-terminal lysine on one or both Fc domains. In some embodiments, the modification includes a K447 deletion (K447_or K447del) in one or both Fc domains. Such modifications are depicted in the heterodimeric Fc backbones of FIGS. 9A-9D.

Exemplary IL18 variants that can be included in the IL18-Fc fusion proteins include, but are not limited to, those in FIGS. 13A-20D, 41A-44C, 87, and 88A-88E. In some embodiments, the IL18-Fc fusion protein includes one or more of the IL18 variants in FIGS. 13A-20D and 41A-44C. In certain embodiments, the IL18-Fc fusion protein includes an IL18 that includes 1, 2, 3, 4, 5, 6, 7, 8, 9, 10 or more additional modifications as compared to an IL18 variant in FIGS. 13A-20D, 41A-44C, 87, and 88A-88E.

In some embodiments, the IL18 variant is engineered to remove a possible “DG” aspartic acid isomerization motif, such as those found when the C-terminus of IL18 is covalently attached to a G4S linker. In certain embodiments, the C-terminus of IL18 is covalently attached to the glutamic acid (E) residue of the hinge region of the Fc domain. In some embodiments, the IL18 variant of the fusion protein comprises a deletion or substitution at the C-terminal aspartic acid (D) residue (D). In certain embodiments, the IL18 variant includes a modification such as a D157del (D157_), D157S, or D157A modification. In some embodiments, the C-terminus of IL18 is covalently attached to a linker selected from the group including: AGGGG (SEQ ID NO: 39) or EAAAK (SEQ ID NO:40).

In some embodiments, the IL18-Fc fusion protein includes a format to improve the production of the fusion protein. In some instances, the IL18-Fc fusion protein includes a Fab-Fc chain occupying the empty-Fc chain. In some embodiments, the Fab arm is a silent Fv. In some embodiments, the silent Fv is based on SEQ ID NOS:23 and 24 as disclosed in the sequence listing of WO 2020/078905. In some embodiments, the format comprising a first monomer comprising a IL18 protein or variant thereof and a first Fe domain, a second monomer comprising the heavy chain of a Fab arm and a second Fc domain, and a third monomer comprising the light chain of the Fab arm.

In some embodiments, the empty-Fc chain of an IL18-Fc fusion protein includes the substitutions H435R/Y436F in the Fc domain. In some embodiments, the empty-Fc chain of the IL18-Fc fusion protein does not include the substitutions H435R/Y436F in the Fc domain. In some embodiments, the Fab-Fc chain of the IL18-Fc fusion protein includes the substitutions H435R/Y436F in the Fc domain. In some embodiments, the Fab-Fc chain of the IL18-Fc fusion protein does not include the substitutions H435R/Y436F in the Fc domain.

In some embodiments, the IL18 variant of the fusion protein includes a modification at position E31 of IL18. In some embodiments, the IL18 variant includes the amino acid substitution E31Q.

In some embodiments, the IL18 variant of the fusion protein includes a modification at position K53 of IL18. In some embodiments, the IL18 variant includes the amino acid substitution K53D. In some embodiments, the IL18 variants includes the amino acid substitutions K53T.

In one aspect, provided herein are compositions that include any one of the IL18 variants described herein.

### VI. Heterodimerization Variants

In some embodiments, the dimeric IL18-Fc fusion protein is a heterodimeric IL18-Fc fusion protein. Such heterodimeric proteins include two different Fc domains (one on each of the first and second monomers) that include modifications that facilitate the heterodimerization of the first and second monomers and/or allow for ease of purification of heterodimers over homodimers, collectively referred to herein as “heterodimerization variants.” As discussed below, heterodimerization variants can include skew variants (e.g., the “knobs and holes” and “charge pairs” variants described below) as well as “pI variants” that facilitates the separation of homodimers away from heterodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

1. Skew Variant

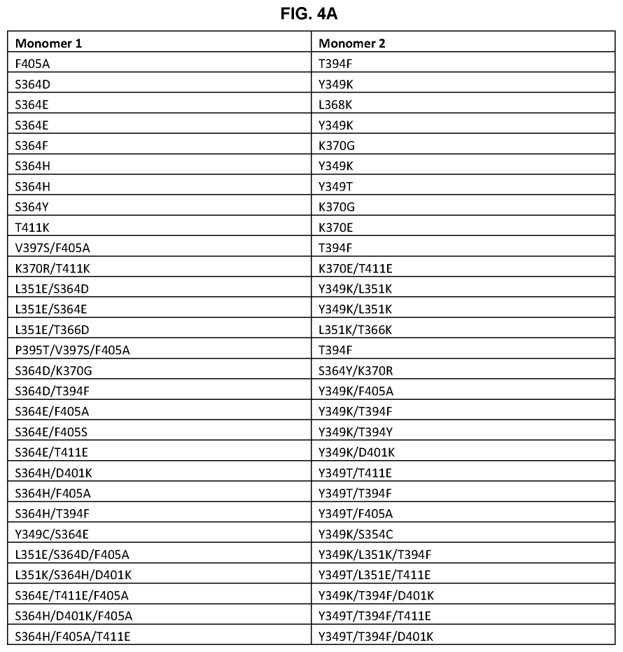

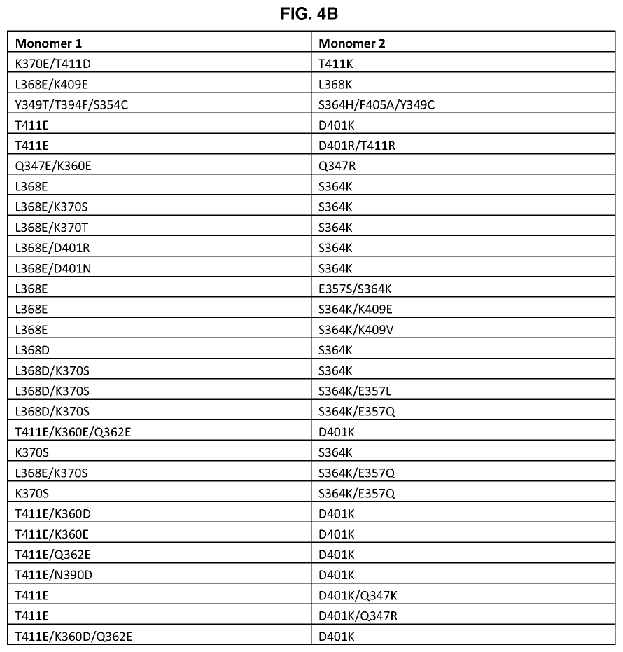

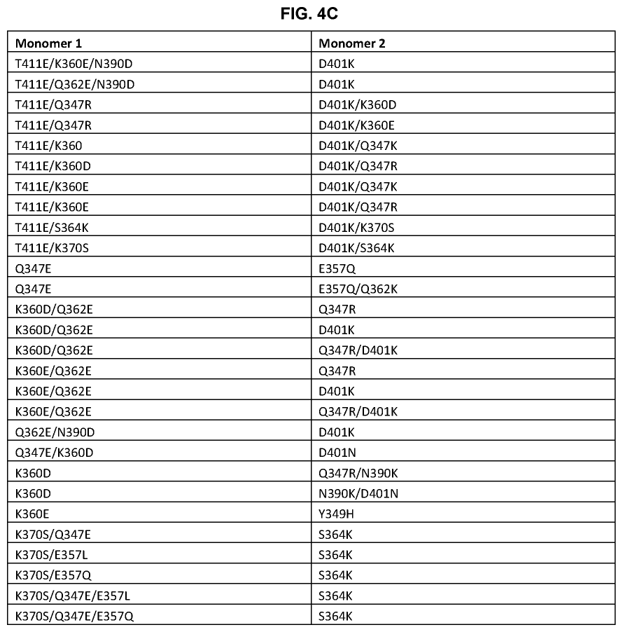

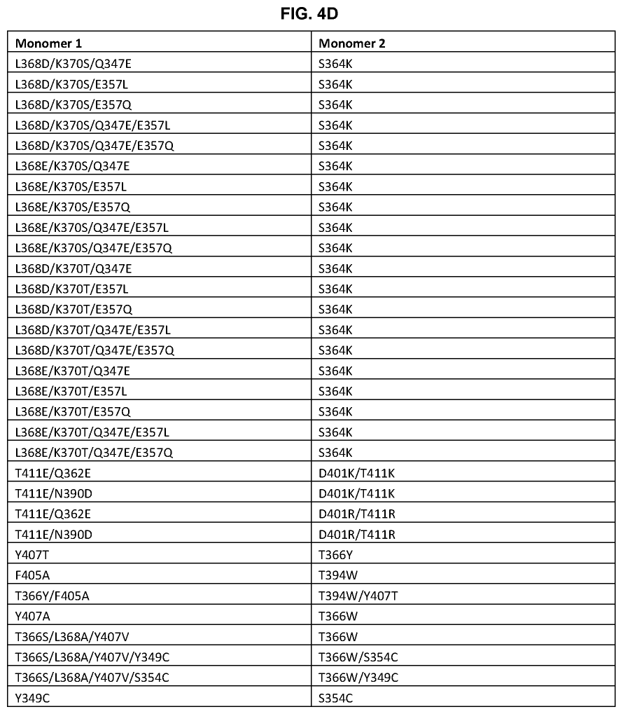

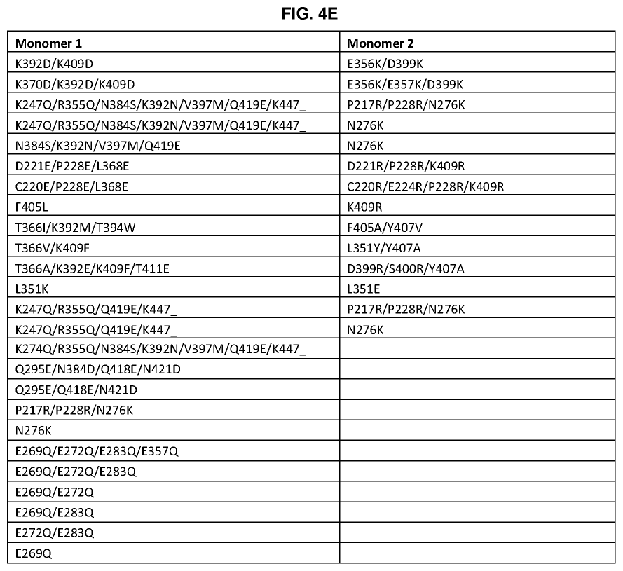

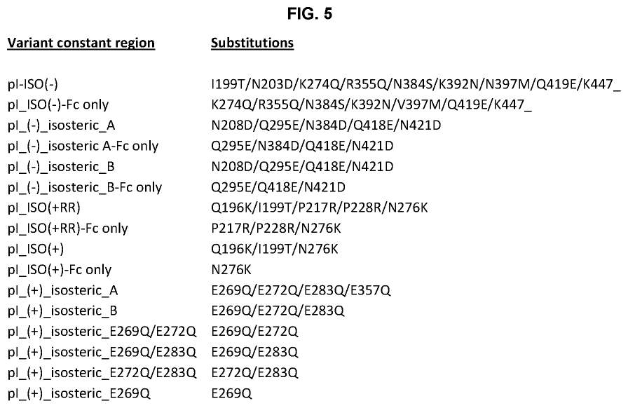

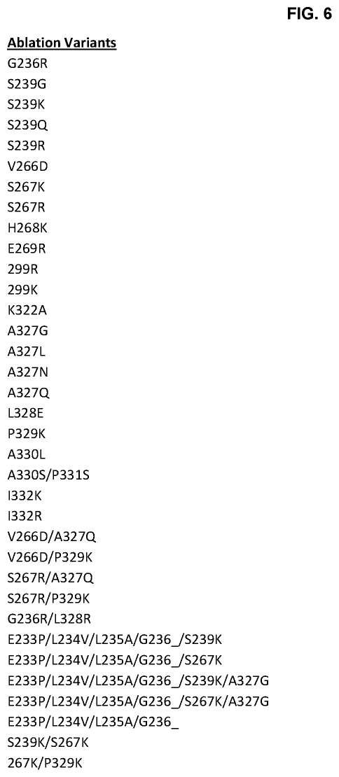

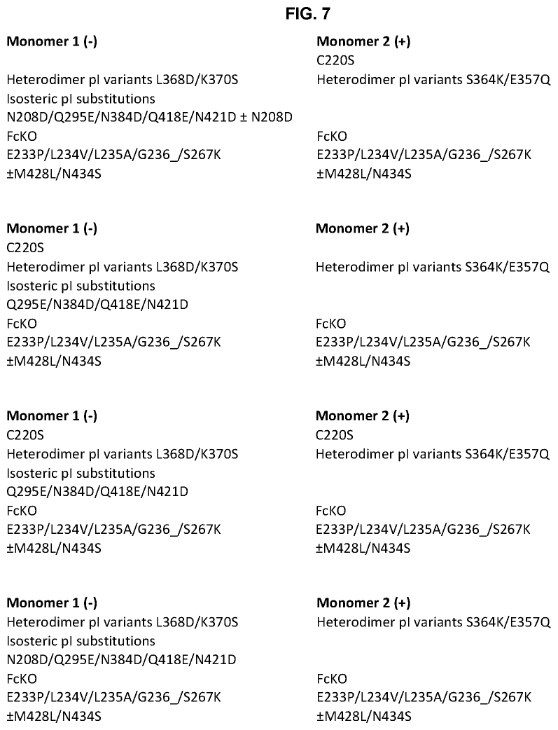

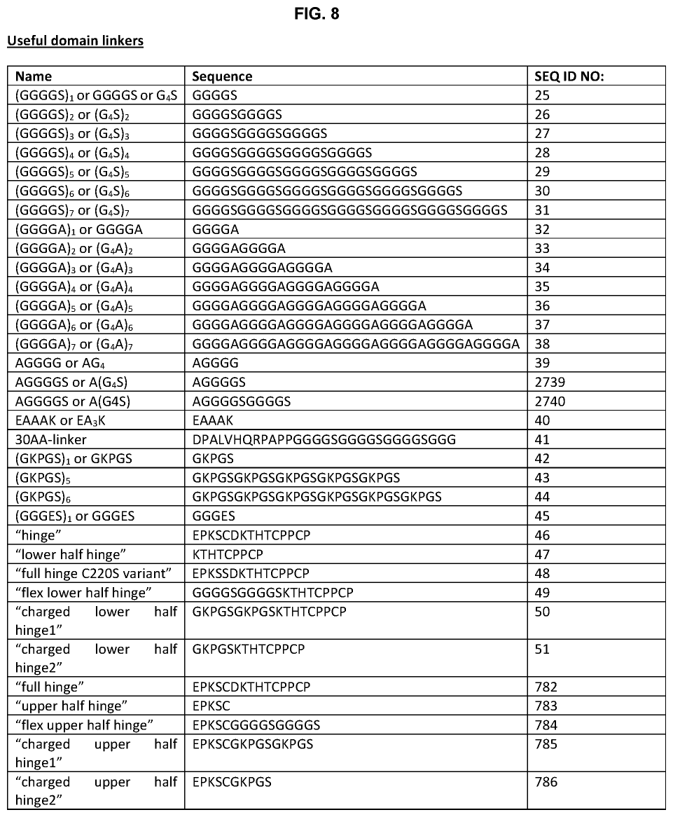

In some embodiments, the heterodimeric IL18-Fc fusion protein includes skew variants, which are one or more amino acid modifications in a first Fc domain (A) and/or a second Fc domain (B) that favor the formation of Fc heterodimers (Fc dimers that include the first and the second Fc domain; A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIG. 4.

One mechanism for skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A—monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

An additional mechanism for skew variants that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer” corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric IL18-Fc fusion proteins include one or more sets of such heterodimerization skew variants. Exemplary skew variants that fall into this category include: S364K/E357Q:L368D/K370S; L368D/K370S:S364K; L368E/K370S:S364K; T411E/K360E/Q362E:D401K; L368D/K370S:S364K/E357L; K370S:S364K/E357Q; a T366S/L368A/Y407V:T366W (optionally including a bridging disulfide, T366S/L368A/Y407V/Y349C:T366W/S354C or T366S/L368A/Y407V/S354C: T366W/Y349C). These variants come in “pairs” of “sets.” That is, one set of the pair is incorporated into the first monomer and the other set of the pair is incorporated into the second monomer. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIG. 4.

In exemplary embodiments, the heterodimeric IL18-Fc fusion protein includes a S364K/E357Q:L368D/K370S; L368D/K370S:S364K; L368E/K370S:S364K; T411E/K360E/Q362E: D401K; L368D/K370S:S364K/E357L; K370S:S364K/E357Q; or a T366S/L368A/Y407V: T366W (optionally including a bridging disulfide, T366S/L368A/Y407V/Y349C: T366W/S354C or T366S/L368A/Y407V/S354C T366W/Y349C) “skew” variant amino acid substitution set. In an exemplary embodiment, the heterodimeric IL18-Fc fusion protein includes a “S364K/E357Q:L368D/K370S” amino acid substitution set.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the IL18-Fc fusion protein. Further, individual modifications can also independently and optionally be included or excluded from the subject IL18-Fc fusion proteins.

2. pI (Isoelectric Point) Variants for Heterodimers

In some embodiments, the heterodimeric IL18-Fc fusion protein includes purification variants that advantageously allow for the separation of heterodimeric IL18-Fc fusion proteins from homodimeric proteins.

There are several basic mechanisms that can lead to ease of purifying heterodimeric proteins. One such mechanism relies on the use of pI variants which include one or more modifications that affect the isoelectric point of one or both of the monomers of the fusion protein, such that each monomer, and subsequently each dimeric species, has a different pI, thus allowing the isoelectric purification of A-A, A-B and B-B dimeric proteins. Alternatively, some formats also allow separation on the basis of size. As is further outlined below, it is also possible to “skew” the formation of heterodimers over homodimers using skew variants. Thus, a combination of heterodimerization skew variants and pI variants find particular use in the subject IL18 fusion proteins provided herein.

Additionally, as more fully outlined below, depending on the format of the heterodimeric Fc fusion protein, pI variants can be either contained within the constant region and/or Fc domains of a monomer, and/or domain linkers can be used. In some embodiments, the heterodimeric IL18-Fc fusion protein includes additional modifications for alternative functionalities can also create pI changes, such as Fc, FcRn and KO variants.

In the embodiments that utilizes pI as a separation mechanism to allow the purification of heterodimeric IL18-Fc fusion proteins, amino acid modifications can be introduced into one or both of the monomers of the heterodimeric IL18-Fc fusion protein. That is, the pI of one of the monomers (referred to herein for simplicity as “monomer A”) can be engineered away from monomer B, or both monomer A and B can be changed, with the pI of monomer A increasing and the pI of monomer B decreasing. As discussed, the pI changes of either or both monomers can be done by removing or adding a charged residue (e.g., a neutral amino acid is replaced by a positively or negatively charged amino acid residue, e.g., glutamine to glutamic acid), changing a charged residue from positive or negative to the opposite charge (e.g. aspartic acid to lysine) or changing a charged residue to a neutral residue (e.g., loss of a charge; lysine to serine.). A number of these variants are shown in the figures, including, FIGS. 4 and 5.

Creating a sufficient change in pI in at least one of the monomers such that heterodimers can be separated from homodimers can be done by using a “wild type” heavy chain constant region and a variant region that has been engineered to either increase or decrease its pI (wt A: B+ or wt A: B−), or by increasing one region and decreasing the other region (A+: B− or A−: B+).

Thus, in general, a component of some embodiments of the present subject fusion proteins are amino acid variants in the Fc domains or constant domain regions that are directed to altering the isoelectric point (pI) of at least one, if not both, of the monomers of a dimeric protein by incorporating amino acid substitutions (“pI variants” or “pI substitutions”) into one or both of the monomers. The separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) of a heterodimeric IL18-Fc fusion protein to achieve good separation will depend in part on the starting pI of the components. That is, to determine which monomer to engineer or in which “direction” (e.g., more positive or more negative), the sequences of the Fc domains and any IL18 or linker included in each monomer are calculated and a decision is made from there based on the pIs of the monomers. As is known in the art, different Fc domains, linkers and IL18s will have different starting pIs. In general, as outlined herein, the pIs are engineered to result in a total pI difference of each monomer of at least about 0.1 logs, with 0.2 to 0.5 being preferred as outlined herein.

In general, as will be appreciated by those in the art, there are two general categories of amino acid modifications that affect pI: those that increase the pI of the protein (basic changes) and those that decrease the pI of the protein (acidic changes). As described herein, all combinations of these variants can be used: one monomer may include a wild type Fc domain, or a variant Fc domain that does not display a significantly different pI from wild-type, and the other monomer includes a Fc domain that is either more basic or more acidic. Alternatively, each monomer may be changed, one to more basic and one to more acidic.

In the case where pI variants are used to achieve heterodimerization, a more modular approach to designing and purifying heterodimeric IL18-Fc fusion proteins is provided. Thus, in some embodiments, heterodimerization variants (including skew and pI variants) must be engineered. In addition, in some embodiments, the possibility of immunogenicity resulting from the pI variants is significantly reduced by importing pI variants from different IgG isotypes such that pI is changed without introducing significant immunogenicity (see isotypic variants below). Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g., Asn to Asp; and Gln to Glu).

A side benefit that can occur with this pI engineering is also the extension of serum half-life and increased FcRn binding. That is, as described in US Publ. App. No. US 2012/0028304 (incorporated by reference in its entirety and specifically for the disclosure of pI variants that provide additional function), lowering the pI of antibody constant domains (including those found in Fc fusions) can lead to longer serum retention in vivo. These pI variants for increased serum half-life also facilitate pI changes for purification.

In addition, it should be noted that the pI variants of the heterodimerization variants give an additional benefit for the analytics and quality control process of Fc fusion proteins, as the ability to either eliminate, minimize and distinguish when homodimers are present is significant. Similarly, the ability to reliably test the reproducibility of the heterodimeric Fc fusion protein production is important.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, the heterodimeric IL18-Fc fusion protein includes a monomer with a variant Fc domain having pI variant modifications 295E/384D/418E/421D (Q295E/N384D/Q418E/N421D when relative to human IgG1). In one embodiment, the heterodimeric IL18-Fc fusion protein includes a monomer with a variant Fc domain having pI variant modifications 217R/228R/276K (P217R/P228R/N276K when relative to human IgG1). Additional exemplary pI variant modification that can be incorporated into the Fc domain of a subject are depicted in FIG. 5.

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an IL18-Fc fusion protein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

3. Isotypic Variants

In addition, some embodiments of the IL18-Fc fusion proteins provided herein rely on the “importation” of pI amino acids at particular positions from one IgG isotype into another, thus reducing or eliminating the possibility of unwanted immunogenicity being introduced into the variants. A number of these are shown in FIG. 21 of US Publ. App. No. 2014/0370013, hereby incorporated by reference, particularly for its disclosure of isotypic variants. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significantly affect the pI of the variant Fc fusion protein. However, it should be noted as discussed below that even changes in IgG2 molecules allow for increased serum half-life.

In other embodiments, non-isotypic amino acid modifications are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant modifications in each monomer of the heterodimer can be seen. As discussed herein, having the pIs of the two monomers differ by at least 0.5 can allow separation by ion exchange chromatography or isoelectric focusing, or other methods sensitive to isoelectric point.

4. Calculating pI

The pI of each monomer of the IL18-Fc fusion protein can depend on the pI of the variant Fc domain and the pI of the total monomer, including the variant Fc domain and any IL18 and/or domain linker included in the monomer. Thus, in some embodiments, the change in pI is calculated on the basis of the variant Fc domain, using the chart in the FIG. 19 of US Publ. App. No. 2014/0370013, hereby incorporated by reference, particularly for its disclosure of methods of calculating pI. As discussed herein, which monomer to engineer is generally decided by the inherent pI of each monomer.

5. pI Variants that Also Confer Better FcRn In Vivo Binding

In the case where the pI variant(s) decreases the pI of the monomer, such modifications can have the added benefit of improving serum retention in vivo.

Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc modifications that will increase Fc's half-life in vivo will ideally increase FcRn binding at the lower pH while still allowing release of Fc at higher pH. The amino acid histidine changes its charge state in the pH range of 6.0 to 7.4. Thus, it is not surprising to find His residues at important positions in the Fc/FcRn complex.

### VII. Other Fc Variants for Additional Functionality

In addition to heterodimerization variants, the subject heterodimeric IL18-Fc fusion proteins provided herein may independently include Fc modifications that affect functionality including, but not limited to, altering binding to one or more Fc receptors (e.g., FcγR and FcRn).

1. FcγR Variants

In one embodiment, the IL18-Fc fusion proteins include one or more amino acid modifications that affect binding to one or more Fcγ receptors (i.e., “FcγR variants”). FcγR variants (e.g., amino acid substitutions) that result in increased binding as well as decreased binding can be useful. For example, it is known that increased binding to FcγRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. FcγR variants that find use in the IL18 fusion proteins include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

In addition, amino acid substitutions that increase affinity for FcγRIIc can also be independently included in the Fc domain variants outlined herein. Useful substitutions that for FcγRIIc are described in, for example, U.S. Pat. Nos. 8,188,321 and 10,113,001, all of which are expressly incorporated herein by reference in their entirety and specifically for the variants disclosed therein that affect Fcγ receptor binding.

2. FcRn Variants

Further, IL18-Fc fusion proteins described herein can independently include Fc substitutions that confer increased binding to the FcRn and increased serum half-life. Such modifications are disclosed, for example, in U.S. Pat. No. 8,367,805, hereby incorporated by reference in its entirety, and specifically for Fc substitutions that increase binding to FcRn and increase half-life. Such modifications include, but are not limited to 434S, 434A, 428L, 308F, 259I, 428L/434S, 428L/434A, M252Y/S254T/T256E, 259I/308F, 436I/428L, 436I or V/434S, 436V/428L and 259I/308F/428L.

3. Ablation Variants

In some embodiments, the IL18-Fc fusion protein includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In some embodiments, particularly in the use of immunomodulatory proteins, it is desirable to ablate FcγRIIIa binding to eliminate or significantly reduce ADCC activity such that one of the Fc domains comprises one or more Fcγ receptor ablation variants. These ablation variants are depicted in FIG. 31 of U.S. Pat. No. 10,259,887, which is herein incorporated by reference in its entirety, and each can be independently and optionally included or excluded, with preferred aspects utilizing ablation variants selected from the group including: G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to the EU index. In addition, ablation variants of use in the subject IL18-Fc fusion proteins are also depicted in FIG. 6. It should be noted that the ablation variants referenced herein ablate FcγR binding but generally not FcRn binding.

### VIII. Combination of Heterodimeric and Fc Variants

As will be appreciated by those in the art, the Fc modifications described herein can independently be combined. For example, all of the recited heterodimerization variants (including skew and/or pI variants) can be optionally and independently combined in any way, as long as they retain their “strandedness” or “monomer partition.”

In the case of pI variants, while embodiments finding particular use are shown in the figures, other combinations can be generated, following the basic rule of altering the pI difference between two monomers to facilitate purification.

In addition, any of the heterodimerization variants, may also be independently and optionally combined with other variants described herein including, but not limited to, Fc ablation variants, FcRn variants, and/or half/life extension variants as generally outlined herein.

Exemplary combinations of modifications are shown in FIG. 7 and the backbone sequences in FIG. 9 (heterodimeric backbones). In certain embodiments, the IL18-Fc fusion protein is heterodimeric and includes a combination of Fc domain modifications as depicted in FIG. 4. In some embodiments, the heterodimeric IL18-Fc fusion protein includes a first monomer having a first Fc domain with the backbone sequence of any one of the “monomer 1” backbones in FIG. 7 and a second Fc domain with the backbone sequence of a corresponding “monomer 2” backbone in FIG. 7. In certain embodiments, the homodimeric IL18-Fc fusion protein includes a first monomer with a first Fc domain and a second monomer with a second Fc domain, where the first and second Fc domains each have the sequence of any of the backbone sequences in FIGS. 9 and 10.

In some embodiments, wherein the IL18-Fc fusion protein is a monovalent (i.e., only one IL18), the first monomer includes a first Fc domain with heterodimer skew variants L368D/K370S, isosteric pI variants Q295E/N384D/Q418E/N421D, and FcKO variants E233P/L234V/L235A/G236del/S267K and the second monomer includes a second Fc domain with heterodimer skew variants S364K/E357Q and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first and second monomers each also include a N297A or N297S amino acid substitution that removes glycosylation. In some embodiments, the first monomer includes a first Fc domain with modifications C220S/E233P/L234V/L235A/G236del/S267K/Q295E/L368D/K370S/N384D/Q418E/N421D and optionally M428L/N434S and the second monomer includes a second Fc domain with modifications C220S/E233P/L234V/L235A/G236del/S267K/S364K/E357Q and optionally modifications M428L/N434S, according to the EU index.

In some embodiments, wherein the IL18-Fc fusion protein is a monovalent (i.e., only one IL18), the first monomer includes a first Fc domain with heterodimer skew variants L368D/K370S, isosteric pI variants Q295E/N384D/Q418E/N421D, and FcKO variants E233P/L234V/L235A/G236del/S267K and the second monomer includes a second Fc domain with heterodimer skew variants S364K and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/E233P/L234V/L235A/G236del/S267K/Q295E/L368D/K370S/N384D/Q418E/N421D and optionally M428L/N434S and the second monomer includes a second Fc domain with modifications C220S/E233P/L234V/L235A/G236del/S267K/S364K and optionally modifications M428L/N434S, according to the EU index.

In some embodiments, wherein the IL18-Fc fusion protein is a monovalent (i.e., only one IL18), the first monomer includes a first Fc domain with heterodimer skew variants L368E/K370S, isosteric pI variants Q295E/N384D/Q418E/N421D, and FcKO variants E233P/L234V/L235A/G236del/S267K and the second monomer includes a second Fc domain with heterodimer skew variants S364K and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/E233P/L234V/L235A/G236del/S267K/Q295E/L368E/K370S/N384D/Q418E/N421D and optionally M428L/N434S and the second monomer includes a second Fc domain with modifications C220S/E233P/L234V/L235A/G236del/S267K/S364K and optionally modifications M428L/N434S, according to the EU index.

In some embodiments, wherein the IL18-Fc fusion protein is a monovalent (i.e., only one IL18), the first monomer includes a first Fc domain with heterodimer skew variants K360E/Q362E/T411E, isosteric pI variants Q295E/N384D/Q418E/N421D, and FcKO variants E233P/L234V/L235A/G236del/S267K and the second monomer includes a second Fc domain with heterodimer skew variants D401K and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/E233P/L234V/L235A/G236del/S267K/Q295E/K360E/Q362E/384D/T411E/Q418E/N421D and optionally M428L/N434S and the second monomer includes a second Fc domain with modifications C220S/E233P/L234V/L235A/G236del/D401K and optionally modifications M428L/N434S, according to the EU index.

In some embodiments, wherein the IL18-Fc fusion protein is a monovalent (i.e., only one IL18), the first monomer includes a first Fc domain with heterodimer skew variants L368D/K370S and a variant that ablates Fab arm exchange, S228P, and the second monomer includes a second Fc domain with heterodimeric pI variants S364K/E357Q and S228P to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/L368D/K370S and optionally M428L/N434S and the second monomer includes a second Fc domain with modifications C220S/S228P/S364K/E357Q and optionally modifications M428L/N434S, according to the EU index. In exemplary embodiments, the Fc domains are human IgG4 Fc domains.

In some embodiments, wherein the IL18-Fc fusion protein is a monovalent (i.e., only one IL18), the first monomer includes a first Fc domain with heterodimer skew variants L368D/K370S and isosteric pI variants Q295E/N384D/Q418E/N421D and the second monomer includes a second Fc domain with heterodimer skew variants S364K/E357Q, according to the EU index. In some embodiments, the first and second monomers each also include M428L/N434S half-life extension variants. In some embodiments, the first and second monomers each also include a C219S hinge modification. In some embodiments, the first monomer includes a first Fc domain with modifications Q295E/L368D/K370S/384D/Q418E/N421D and optionally M428L/N434S and the second monomer includes a second Fc domain with modifications S364K/E357Q and optionally modifications M428L/N434S, according to the EU index. In exemplary embodiments, the Fc domains are human IgG2 Fc domains.

In some embodiments, wherein the IL18-Fc fusion protein is a monovalent (i.e., only one IL18), the first monomer includes a first Fc domain with heterodimer skew variants L368D/K370S, isosteric pI variants Q295E/N384D/Q418E/N421D, and FcKO variant S267K and the second monomer includes a second Fc domain with heterodimer skew variants S364K/E357Q and FcKO variant S267K, according to the EU index. In some embodiments, the first and second monomers each also include M428L/N434S half-life extension variants. In some embodiments, the first and second monomers each also include a C219S hinge modification. In some embodiments, the first monomer includes a first Fc domain with modifications S267K/Q295E/L368D/K370S/384D/Q418E/N421D and optionally M428L/N434S and the second monomer includes a second Fc domain with modifications S267K/S364K/E357Q and optionally modifications M428L/N434S, according to the EU index. In exemplary embodiments, the Fc domains are human IgG2 Fc domains.

In some embodiments, wherein the IL18-Fc fusion protein is a monovalent (i.e., only one IL18), the first monomer includes a first Fc domain with heterodimer skew variants L368D/K370S and FcKO variants E233P/L234V/L235A/G236del/S267K and the second monomer includes a second Fc domain with heterodimer skew variants S364K/E357Q, isosteric pI variants P217R/P228R/N276K, and FcKO variants E233P/L234V/L235A/G236del/S267K, according to the EU index. In some embodiments, the first and second monomers each also include M428L/N434S half-life extension variants. In some embodiments, the first and second monomers each also include a C220S hinge amino acid substitution. In some embodiments, the first monomer includes a first Fc domain with modifications C220S/E233P/L234V/L235A/G236del/S267K/L368D/K370S and optionally M428L/N434S and the second monomer includes a second Fc domain with modifications P217R/C220S/P228R/E233P/L234V/L235A/G236del/S267K/N276K/S364K/E357Q and optionally modifications M428L/N434S, according to the EU index.

The variant Fc domains provided herein can also include 1, 2, 3, 4, 5, 6, 7, 8, 9, or 10 additional mutations in addition to the enumerated mutations.

### IX. Domain Linkers

In some embodiments of the subject IL18-Fc fusion protein, an IL18 is covalently attached to an Fc domain by a linker (e.g., (IL18)₁-L-Fc). In some embodiments, the linker is a “domain linker.” While any suitable linker can be used, many embodiments utilize a glycine-serine polymer, including for example (GS)ₙ, (GSGGS)ₙ [SEQ ID NO:778], (GGGGS)ₙ [SEQ ID NO: 777], and (GGGS)ₙ [SEQ ID NO:779], where n is an integer of at least 0 (and generally from 0 to 1 to 2 to 3 to 4 to 5), as well as any peptide sequence that allows for recombinant attachment of the two domains with sufficient length and flexibility to allow each domain to retain its biological function. In certain cases, useful linkers include (GGGGS)₁ (SEQ ID NO: 25) or (GGGGS)₂ (SEQ ID NO: 26). Illustrative domain linkers are depicted in FIG. 8. In some cases, and with attention being paid to “strandedness”, as outlined below, charged domain linkers can be used as discussed herein.

### X. Additional Antibody Components

In some embodiments, the IL18-Fc fusion constructs provided herein include one or more additional antibody components including but not limited to antigen binding domains (ABDs). In some embodiments, the IL18 fusion constructs described herein include a human TL18 protein or a variant thereof, a CD3 antigen binding domain, and a tumor target antigen (TTA) antigen binding domain. In some embodiments, the trispecific constructs of the invention include a human IL18 protein or a variant thereof, an ABD that binds the ECD of human CD3, and an ABD that binds the extracellular domains of a human tumor target antigen (TTA) selected from the group including: EGFR, Trop2, CD20, B7H3, FLT3, CD19, CD123, CD22, CD38, CEA, MSLN, BCMA, CAIX, CLDN18.2, HER2, PD-1 and ANO1.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to antibodies or antibody fragments (antibody monomers) that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype.

In addition, many of the monomer sequences herein have at least one the cysteines at position 220 replaced by a serine, to reduce disulfide formation. Specifically included within the sequences herein are one or both of these cysteines replaced (C220S).

Thus, “isotype” as used herein is meant any of the subclasses of immunoglobulins defined by the chemical and antigenic characteristics of their constant regions.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3).

A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF MIMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat. As shown herein and described below, the pI variants can be in one or more of the CH regions, as well as the hinge region, discussed below.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus, for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230. As noted herein, pI variants can be made in the hinge region as well.

The light chain generally comprises two domains, the variable light domain (containing the light chain CDRs and together with the variable heavy domains forming the Fv region), and a constant light chain region (often referred to as CL or Cκ).

Another region of interest for additional substitutions, outlined herein, is the Fc region.

Thus, the present heterodimeric fusion proteins provided herein include one or more antibody domains. As described herein and known in the art, the heterodimeric antibodies provided herein comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

In certain embodiments, the ABDs of the invention comprise a heavy chain variable region with frameworks from a particular germline heavy chain immunoglobulin gene and/or a light chain variable region from a particular germline light chain immunoglobulin gene. For example, such ABDs may comprise or consist of a human ABD comprising heavy or light chain variable regions that are “the product of” or “derived from” a particular germline sequence. An ABD that is “the product of” or “derived from” a human germline immunoglobulin sequence can be identified as such by comparing the amino acid sequence of the ABD to the amino acid sequences of human germline immunoglobulins and selecting the human germline immunoglobulin sequence that is closest in sequence (i.e., greatest % identity) to the sequence of the ABD. An ABD that is “the product of” or “derived from” a particular human germline immunoglobulin sequence may contain amino acid differences as compared to the germline sequence, due to, for example, CDRs, naturally occurring somatic mutations or intentional introduction of site-directed mutation. However, a humanized ABD typically is at least 90% identical in amino acids sequence to an amino acid sequence encoded by a human germline immunoglobulin gene and contains amino acid residues that identify the ABD as being derived from human sequences when compared to the germline immunoglobulin amino acid sequences of other species (e.g., murine germline sequences). In certain cases, a humanized ABD may be at least 95, 96, 97, 98 or 99%, or even at least 96%, 97%, 98%, or 99% identical in amino acid sequence to the amino acid sequence encoded by the germline immunoglobulin gene. Typically, a humanized ABD derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized ABD may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention). In one embodiment, the parent ABD has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference. The ABD herein can be in the form of either a Fab or an scFv.

1. CD3 Antigen Binding Domains

In some embodiments, the antibody component is a CD3 ABD. The CD3 ABD can be in the form of either a Fab or an scFv. In some embodiments, the CD3 ABD is a scFv, wherein the VH and VL domains are joined using an scFv linker, which can be optionally a charged scFv linker. As will be appreciated by those in the art, the scFv can be assembled from N- to C-terminus, as N-VH-scFv linker-VL-C or as N-VL-scFv linker-VH-C, with the C terminus of the scFv domain generally being linked to the hinge-CH2-CH3 Fc domain, wherein the hinge in this case serving as a domain linker. Suitable Fvs (including CDR sets and variable heavy/variable light domains) can be used in scFv formats or Fab formats are shown in FIG. 120.

Alternatively, the CD3 ABD can be in the form of a Fab fragment. In this embodiment, the ABD is made up of a variable heavy domain, contributed by a heavy chain, and a variable light domain, contributed by a light chain. Suitable Fvs (including CDR sets and variable heavy/variable light domains) can be used in scFv formats or Fab formats are shown in the FIG. 120.

As will be appreciated by those in the art, suitable CD3 binding domains can comprise a set of 6 CDRs as depicted in the sequence listing and FIG. 120, either as they are underlined/bolded or, in the case where a different numbering scheme is used as described herein and as shown in Table 2, as the CDRs that are identified using other alignments within the variable heavy (VH) domain and variable light domain (VL) sequences of those depicted in the FIG. 109 and the sequence listing. Suitable CD3 ABDs that find use in the subject IL18-Fc fusion proteins can also include the entire VH and VL sequences as depicted in these sequences and figures, used as scFvs or as Fabs.

In one embodiment, the CD3 antigen binding domain includes the 6 CDRs (i.e., vhCDR1-3 and vlCDR1-3) of any of the CD3 binding domains described in FIG. 109 or the sequence listing.

In addition to the parental CDR sets disclosed in the figures and sequence listing that form an ABD to CD3, provided herein are variant CD3 ABDS having CDRs that include at least one modification of the CD3 ABD CDRs disclosed herein (e.g., FIG. 109). In one embodiment, the heterodimeric fusion protein includes a CD3 ABD that includes a set of 6 CDRs with 1, 2, 3, 4, 5, 6, 7, 8, 9, 10 amino acid modifications as compared to the 6 CDRs of a CD3 ABD as depicted in FIG. 109 or the sequence listing. In certain embodiments, the CD3 ABD is capable of binding CD3 antigen, as measured by at least one of a Biacore, surface plasmon resonance (SPR) and/or BLI (biolayer interferometry, e.g., Octet assay) assay, with the latter finding particular use in many embodiments.

In one embodiment, the CD3 ABD of the subject IL18-Fc fusion protein includes 6 CDRs that are at least 90, 95, 97, 98 or 99% identical to the 6 CDRs of a CD3 ABD as depicted in FIG. 120 or the sequence listing. In certain embodiments, the CD3 ABD is capable of binding to the CD3, as measured by at least one of a Biacore, surface plasmon resonance (SPR) and/or BLI (biolayer interferometry, e.g., Octet assay) assay, with the latter finding particular use in many embodiments.

In some embodiments, the CD3 ABD of the IL18-Fc fusion protein includes the variable heavy domain (VH) and variable light domain (VL) of any of the CD3-ABDs disclosed herein, including, but not limited to those disclosed in FIG. 120. In addition to the parental CD3 variable heavy and variable light domains disclosed herein, provided herein are subject IL18-Fc fusion proteins having one or more CD3 ABDs that include a variable heavy domain and/or a variable light domain that are variants of a CD3 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD3 ABD depicted in FIG. 120 or the sequence listing. In certain embodiments, the CD3 ABD is capable of binding to CD3, as measured at least one of a Biacore, surface plasmon resonance (SPR) and/or BLI (biolayer interferometry, e.g., Octet assay) assay, with the latter finding particular use in many embodiments.

In one embodiment, the variant VH and/or VL domain of the IL18-Fc fusion protein is at least 90, 95, 97, 98 or 99% identical to the VH and/or VL of a CD3 ABD as depicted in FIG. 120 or the sequence listing. In certain embodiments, the CD3 ABD is capable of binding to CD3, as measured by at least one of a Biacore, surface plasmon resonance (SPR) and/or BLI (biolayer interferometry, e.g., Octet assay) assay, with the latter finding particular use in many embodiments.

2. Tumor Target Antigen Binding Domains

Described herein are trispecific constructs that include a human IL18 protein or a variant thereof, a CD3 antigen binding domain, and a tumor target antigen (TTA) antigen binding domain.

The present invention provides constructs that bind to the extracellular domains of human tumor target antigens (TTAs), including, but not limited to, EGFR, Trop2, CD20, B7H3, FLT3, CD19, CD123, CD22, CD38, CEA, MSLN, BCMA, CAIX, CLDN18.2, HER2, PD-1 and ANO1. Binding affinities of a specific TTA ABD can be evaluated using an assay recognized by those skilled in the art including, but not limited to, a Surface Plasmon Resonance (SPR) and/or a BLI binding assay (such as Biacore, Octet, or Carterra LSA).

a. B7H3 Antigen Binding Domain

In some embodiments, the trispecific constructs of the invention include an ABD that binds to the ECD of human B7H3. Suitable variable heavy and variable light domains that bind to the ECD of human B7H3 include, but are not limited to, the sequences shown in FIGS. 146, 147, 148A-148H, and 149A-149R. Additionally, any sequence included in FIGS. 146, 147, 148A-148H, and 149A-149R can also be individually included or excluded. It should be noted that these ABDs can be included in any of the heterodimeric formats described herein.

Additionally, included herein are B7H3 ABDs that have the variable heavy chain depicted in SEQ ID NO: 11 or SEQ ID NO: 13, and variable light chain depicted in SEQ ID NO: 12 or SEQ ID NO: 14 from U.S. Pat. No. 10,501,544, incorporated by reference in its entirety, with particularity for relevant disclosure pertaining to B7H3 ABDs and the accompanying sequences described therein. Additionally, any sequence included above from U.S. Pat. No. 10,501,544 can also be individually included or excluded. Further, included herein are B7H3 antigen binding domain with: a) the VH CDRs depicted in SEQ ID NO: 1, SEQ ID NO: 25, and SEQ ID NO: 33 in combination with the VL CDRs depicted in SEQ ID NO: 34, SEQ ID NO: 36, and SEQ ID NO: 6; or b) the VH CDRs depicted in SEQ ID NO: 1, SEQ ID NO: 25, and SEQ ID NO: 10 in combination with the VL CDRs depicted in SEQ ID NO: 38, SEQ ID NO: 30, and SEQ ID NO: 6; both from U.S. Pat. No. 9,963,509, incorporated by reference in its entirety, with particularity for relevant disclosure pertaining to B7H3 ABDs and the accompanying sequences described therein. Additionally, any sequence included above from U.S. Pat. No. 9,963,509 can also be individually included or excluded. Further, included herein are anti-B7H3 antigen binding domains with the VH CDRs depicted in SEQ ID NO: 1, SEQ ID NO: 9, and SEQ ID NO: 10 in combination with the VL CDRs depicted in SEQ ID NO: 11, SEQ ID NO: 312, and SEQ ID NO: 6; from U.S. Pat. No. 10,865,245, incorporated by reference in its entirety, with particularity for relevant disclosure pertaining to B7H3 ABDs and the accompanying sequences described therein. Additionally, any sequence included above from U.S. Pat. No. 10,865,245 can also be individually included or excluded. Still further, included herein are anti-B7H3 antigen binding domain with: a) vhCDR1 with the sequence depicted in SEQ ID NO: 110; b) vhCDR2 with the sequence depicted in SEQ ID NO: 111; c) vhCDR3 with the sequence depicted in SEQ ID NO: 113; d) vlCDR1 with the sequence depicted in SEQ ID NO: 114; e) vlCDR2 with the sequence depicted in SEQ ID NO: 115; and f) vlCDR3 with the sequence depicted in SEQ ID NO: 116, from WO2020/033702, incorporated by reference in its entirety, with particularity for relevant disclosure pertaining to B7H3 ABDs and the accompanying sequences described therein. Additionally, any sequence included above from WO2020/033702 can also be individually included or excluded. Yet further, included herein is an anti-B7H3 antigen binding domain with: a) vhCDR1 with the sequence depicted in SEQ ID NO: 118; b) vhCDR2 with the sequence depicted in SEQ ID NO: 119; c) vhCDR3 with the sequence depicted in SEQ ID NO: 120; d) vlCDR1 with the sequence depicted in SEQ ID NO: 121; e) vlCDR2 with the sequence depicted in SEQ ID NO: 122; and f) vlCDR3 with the sequence depicted in SEQ ID NO: 123, from WO2020/033702, incorporated by reference in its entirety, with particularity for relevant disclosure pertaining to B7H3 ABDs and the accompanying sequences described therein. Additionally, any sequence included above from WO2020/033702 can also be individually included or excluded. Still even further, included herein is an anti-B7H3 antigen binding domain with: a) vhCDR1 with the sequence depicted in SEQ ID NO: 371; b) vhCDR2 with the sequence depicted in SEQ ID NO: 372; c) vhCDR3 with the sequence depicted in SEQ ID NO: 373; d) vlCDR1 with the sequence depicted in SEQ ID NO: 374; e) vlCDR2 with the sequence depicted in SEQ ID NO: 375; and f) vlCDR3 with the sequence depicted in SEQ ID NO: 376, from WO2020/033702, incorporated by reference in its entirety, with particularity for relevant disclosure pertaining to B7H3 ABDs and the accompanying sequences described therein. Additionally, any sequence included above from WO2020/033702 can also be individually included or excluded. Yet still even further, included herein is an anti-B7H3 antigen binding domain comprising: a) a heavy chain variable region comprising a vhCDR1 having SEQ ID NO: 6, a vhCDR2 having SEQ ID NO: 7, and a vhCDR3 having SEQ ID NO: 8; and b) a light chain variable region comprising a vlCDR1 having SEQ ID NO: 9, a vlCDR2 with the sequence WAS (tryptophan-alanine-serine) and a vlCDR3 having SEQ ID NO: 10, the sequences are depicted in WO2022/105879, hereby incorporated by reference for these sequences. Further, included herein is an anti-B7H3 antigen binding domain comprising a variable heavy chain and a variable light chain pair selected from the group including: a) a VH having SEQ ID NO:1 and a VL having SEQ ID NO:5; b) a VH having SEQ ID NO:11 and a VL having SEQ ID NO:15; c) a VH having SEQ ID NO:21 and a VL having SEQ ID NO:25; d) a VH having SEQ ID NO:31 and a VL having SEQ ID NO:35; e) a VH having SEQ ID NO:41 and a VL having SEQ ID NO:45; f) a VH having SEQ ID NO:55 and a VL having SEQ ID NO:57; g) a VH having SEQ ID NO:63 and a VL having SEQ ID NO:35; and h) a VH having SEQ ID NO:63 and a VL having SEQ ID NO:68, wherein the sequences are as depicted in U.S. Pat. No. 11,071,788, hereby incorporated by reference for these sequences. Additionally, any sequence included above from U.S. Pat. No. 11,071,788 can also be individually included or excluded.

In some embodiments, the trispecific constructs of the invention include at least one anti-B7H3 antigen binding domain, as described herein and in the Figures. Described herein is a plurality of means for binding the ECD of human B7H3.

b. EGFR Antigen Binding Domains

In some embodiments, the trispecific constructs of the invention include an ABD that binds to the ECD of human EGFR. Suitable variable heavy and variable light domains that bind to the ECD of human EGFR include, but are not limited to, the sequences shown in FIGS. 150A-150C. Additionally, any sequence included in FIG. 150 can also be individually included or excluded. It should be noted that these ABDs can be included in any of the formats described herein.

Further, included herein is an anti-EGFR antigen binding domain with sequences selected from the group including: a) a VH shown in SEQ ID NO:11 and VL shown in SEQ ID NO:13; b) VH shown in SEQ ID NO:11 and VL shown in SEQ ID NO:16; c) a VH shown in SEQ ID NO:32 and VL shown in SEQ ID NO:33; and d) VH shown in SEQ ID NO:1 and VL shown in SEQ ID NO:16, wherein the sequences are as shown in WO2022/031935, hereby expressly incorporated by reference for the sequences. Additionally, any sequence included above from WO2022/031935 can also be individually included or excluded.

In some embodiments, the trispecific constructs of the invention include at least one anti-EGFR antigen binding domain, as described herein and in the Figures. Described herein is a plurality of means for binding the ECD of human EGFR.

c. HER2 Antigen Binding Domains

In some embodiments, the TTA binds to the ECD of human HER2. Suitable variable heavy and variable light domains that bind to the ECD of human HER2, include, but are not limited to, the sequences shown in FIGS. 151A-151B. Additionally, any sequence included in FIG. 151 can also be individually included or excluded. It should be noted that these ABDs can be included in any of the formats described herein.

In some embodiments, the trispecific constructs of the invention include at least one anti-HER2 antigen binding domain, as described herein and in the Figures. Described herein is a plurality of means for binding the ECD of human HER2.

d. CD19 Antigen Binding Domains

In some embodiments, the TTA binds to the ECD of human CD19. Suitable variable heavy and variable light domains that bind to the ECD of human CD19, include, but are not limited to, the sequences shown in FIG. 152. Additionally, any sequence included in FIG. 152 can also be individually included or excluded. It should be noted that these ABDs can be included in any of the formats described herein.

In some embodiments, the trispecific constructs of the invention include at least one anti-CD19 antigen binding domain, as described herein and in the Figures. Described herein is a plurality of means for binding the ECD of human CD16.

e. CD20 Antigen Binding Domains

In some embodiments, the trispecific constructs include an ABD that binds to the ECD of human CD20. Suitable variable heavy and variable light domains that bind to the ECD of human CD20, but are not limited to, the sequences shown in FIGS. 153A-153C. Additionally, any sequence included in FIG. 153 can also be individually included or excluded. It should be noted that these ABDs can be included in any of the formats described herein.

In some embodiments, the trispecific constructs of the invention include at least one anti-CD20 antigen binding domain, as described herein and in the Figures. Described herein is a plurality of means for binding the ECD of human CD20.

f. CD123 Antigen Binding Domains

In some embodiments, the TTA binds to the ECD of human CD123. Suitable variable heavy and variable light domains that bind to the ECD of human CD123, include, but are not limited to, the sequences shown in FIG. 154B. Additionally, any sequence included in FIG. 154B can also be individually included or excluded. It should be noted that these ABDs can be included in any of the formats described herein.

In some embodiments, the trispecific constructs of the invention include at least one anti-CD123 antigen binding domain, as described herein and in the Figures. Described herein is a means for binding the ECD of human CD123 antigen.

g. CAIX Antigen Binding Domains

In some embodiments, the tumor target antigen (TTA) for use herein bind to the ECD of human carbonic anhydrase 9 (CAIX or CA9). In this case, the amino acid sequences of the variable heavy and variable light chain can be selected from the group including: the amino acid sequences depicted in SEQ ID NO:72 and SEQ ID NO:73 of WO2018/157147; SEQ ID NO:80 and SEQ ID NO:81 as depicted in WO2018/157147, and SEQ ID NO:88 and SEQ ID NO:89 as depicted in WO2018/157147; the heavy and light variable domains of girentuximab, shown in FIG. 23; SEQ ID NO:9 and SEQ ID NO:10 as depicted in US2009/0162382; and SEQ ID NO:17 and SEQ ID NO:19 as depicted in US2009/0162382. Additionally, any sequence described in this paragraph can also be individually included or excluded.

In some embodiments, the trispecific constructs of the invention include at least one anti-CAIX antigen binding domain, as described herein and in FIG. 154A. Described herein is a plurality of means for binding the ECD of human CAIX.

h. FLT3 Antigen Binding Domains

In some embodiments, the trispecific constructs include an ABD that binds to the ECD of human FLT3. Suitable variable heavy and variable light domains that bind to the ECD of human FLT3, but are not limited to, the sequences shown in FIG. 155. Additionally, any sequence included in FIG. 155 can also be individually included or excluded. It should be noted that these ABDs can be included in any of the formats described herein.

Further, included herein is an anti-FLT3 antigen binding domain with a VH/VL pair selected from the group including: a) a variable heavy domain with SEQ ID NO: 187 and a variable light domain with SEQ ID NO: 188; b) a variable heavy domain with SEQ ID NO: 207 and a variable light domain with SEQ ID NO: 208; c) a variable heavy domain with SEQ ID NO: 217 and a variable light domain with SEQ ID NO: 218; d) a variable heavy domain with SEQ ID NO: 227 and a variable light domain with SEQ ID NO: 228; e) a variable heavy domain with SEQ ID NO: 257 and a variable light domain with SEQ ID NO: 258; f) a variable heavy domain with SEQ ID NO: 267 and a variable light domain with SEQ ID NO: 268; g) a variable heavy domain with SEQ ID NO: 277 and a variable light domain with SEQ ID NO: 278; h) a variable heavy domain with SEQ ID NO: 287 and a variable light domain with SEQ ID NO: 288; i) a variable heavy domain with SEQ ID NO: 297 and a variable light domain with SEQ ID NO: 298; j) a variable heavy domain with SEQ ID NO: 307 and a variable light domain with SEQ ID NO: 308; k) a variable heavy domain with SEQ ID NO: 317 and a variable light domain with SEQ ID NO: 318; 1) a variable heavy domain with SEQ ID NO: 337 and a variable light domain with SEQ ID NO: 338; m) a variable heavy domain with SEQ ID NO: 347 and a variable light domain with SEQ ID NO: 348; n) a variable heavy domain with SEQ ID NO: 437 and a variable light domain with SEQ ID NO: 438; o) a variable heavy domain with SEQ ID NO: 457 and a variable light domain with SEQ ID NO: 458; p) a variable heavy domain with SEQ ID NO: 467 and a variable light domain with SEQ ID NO: 468; q) a variable heavy domain with SEQ ID NO: 477 and a variable light domain with SEQ ID NO: 478; r) a variable heavy domain with SEQ ID NO: 497 and a variable light domain with SEQ ID NO: 498; s) a variable heavy domain with SEQ ID NO: 537 and a variable light domain with SEQ ID NO: 538; t) a variable heavy domain with SEQ ID NO: 547 and a variable light domain with SEQ ID NO: 548; u) a variable heavy domain with SEQ ID NO: 567 and a variable light domain with SEQ ID NO: 568; v) a variable heavy domain with SEQ ID NO: 597 and a variable light domain with SEQ ID NO: 598; w) a variable heavy domain with SEQ ID NO: 617 and a variable light domain with SEQ ID NO: 618; x) a variable heavy domain with SEQ ID NO: 637 and a variable light domain with SEQ ID NO: 638; y) a variable heavy domain with SEQ ID NO: 707 and a variable light domain with SEQ ID NO: 708; all as depicted in U.S. Pat. No. 11,447,567 and incorporated by reference herein. Additionally, any sequence included above from U.S. Pat. No. 11,447,567 can also be individually included or excluded.

Further, included herein is an anti-FLT3 antigen binding domain with a VH/VL pair selected from the group including: a) a variable heavy domain with SEQ ID NO: 20 and a variable light domain with SEQ ID NO: 19; b) a variable heavy domain with SEQ ID NO: 6 and a variable light domain with SEQ ID NO: 5; c) a variable heavy domain with SEQ ID NO: 4 and a variable light domain with SEQ ID NO: 3; d) a variable heavy domain with SEQ ID NO: 8 and a variable light domain with SEQ ID NO: 7; e) a variable heavy domain with SEQ ID NO: 10 and a variable light domain with SEQ ID NO: 9; f) a variable heavy domain with SEQ ID NO: 12 and a variable light domain with SEQ ID NO: 11; g) a variable heavy domain with SEQ ID NO: 14 and a variable light domain with SEQ ID NO: 13; h) a variable heavy domain with SEQ ID NO: 16 and a variable light domain with SEQ ID NO: 15; i) a variable heavy domain with SEQ ID NO: 18 and a variable light domain with SEQ ID NO: 17; j) a variable heavy domain with SEQ ID NO: 2 and a variable light domain with SEQ ID NO: 1; k) a variable heavy domain with SEQ ID NO: 22 and a variable light domain with SEQ ID NO: 21; 1) a variable heavy domain with SEQ ID NO: 24 and a variable light domain with SEQ ID NO: 23; m) a variable heavy domain with SEQ ID NO: 26 and a variable light domain with SEQ ID NO: 25; n) a variable heavy domain with SEQ ID NO: 28 and a variable light domain with SEQ ID NO: 27; o) a variable heavy domain with SEQ ID NO: 30 and a variable light domain with SEQ ID NO: 29; p) a variable heavy domain with SEQ ID NO: 32 and a variable light domain with SEQ ID NO: 31; q) a variable heavy domain with SEQ ID NO: 34 and a variable light domain with SEQ ID NO: 33; r) a variable heavy domain with SEQ ID NO: 36 and a variable light domain with SEQ ID NO: 35; s) a variable heavy domain with SEQ ID NO: 205 and a variable light domain with SEQ ID NO: 204; t) a variable heavy domain with SEQ ID NO: 207 and a variable light domain with SEQ ID NO: 206; u) a variable heavy domain with SEQ ID NO: 209 and a variable light domain with SEQ ID NO: 208; v) a variable heavy domain with SEQ ID NO: 211 and a variable light domain with SEQ ID NO: 210; w) a variable heavy domain with SEQ ID NO: 213 and a variable light domain with SEQ ID NO: 212; x) a variable heavy domain with SEQ ID NO: 215 and a variable light domain with SEQ ID NO: 214; y) a variable heavy domain with SEQ ID NO: 217 and a variable light domain with SEQ ID NO: 216; z) a variable heavy domain with SEQ ID NO: 219 and a variable light domain with SEQ ID NO: 218; aa) a variable heavy domain with SEQ ID NO: 221 and a variable light domain with SEQ ID NO: 220; bb) a variable heavy domain with SEQ ID NO: 223 and a variable light domain with SEQ ID NO: 222; cc) a variable heavy domain with SEQ ID NO: 225 and a variable light domain with SEQ ID NO: 224; dd) a variable heavy domain with SEQ ID NO: 227 and a variable light domain with SEQ ID NO: 226; ee) a variable heavy domain with SEQ ID NO: 229 and a variable light domain with SEQ ID NO: 228; ff) a variable heavy domain with SEQ ID NO: 231 and a variable light domain with SEQ ID NO: 230 and gg) a variable heavy domain with SEQ ID NO: 233 and a variable light domain with SEQ ID NO: 232, all as shown in U.S. Pat. No. 11,421,040, hereby incorporated by reference for the sequences. Additionally, any sequence included above from U.S. Pat. No. 11,421,040 can also be individually included or excluded.

Further, included herein is an anti-FLT3 antigen binding domain (“CHv62.21”) with, the variable heavy domain depicted as part of SEQ ID NO: 11, and the variable light domain depicted as part of SEQ ID NO: 10 from U.S. Pat. No. 10,751,422, hereby incorporated by reference for the sequences. Additionally, any sequence included above from U.S. Pat. No. 10,751,422 can also be individually included or excluded.

Further, included herein is an anti-FLT3 antigen binding domain with a VH/VL pair selected from the group including: a) a variable heavy domain with SEQ ID NO: 9 and a variable light domain with SEQ ID NO: 10; b) a variable heavy domain with SEQ ID NO: 13 and a variable light domain with SEQ ID NO: 10; c) a variable heavy domain with SEQ ID NO: 17 and a variable light domain with SEQ ID NO: 10; d) a variable heavy domain with SEQ ID NO: 9 and a variable light domain with SEQ ID NO: 22; e) a variable heavy domain with SEQ ID NO: 37 and a variable light domain with SEQ ID NO: 38; f) a variable heavy domain with SEQ ID NO: 41 and a variable light domain with SEQ ID NO: 42; g) a variable heavy domain with SEQ ID NO: 45 and a variable light domain with SEQ ID NO: 42; h) a variable heavy domain with SEQ ID NO: 49 and a variable light domain with SEQ ID NO: 42; and i) a variable heavy domain with SEQ ID NO: 53 and a variable light domain with SEQ ID NO: 42; all as shown in WO2021/076564, hereby incorporated by reference for the sequences. Additionally, any sequence included above from WO2021/076564 can also be individually included or excluded.

Further, included herein is an anti-FLT3 antigen binding domain with a VH/VL pair selected from the group including: a) a variable heavy domain with SEQ ID NO: 187 and a variable light domain with SEQ ID NO: 288; b) a variable heavy domain with SEQ ID NO: 207 and a variable light domain with SEQ ID NO: 208; c) a variable heavy domain with SEQ ID NO: 217 and a variable light domain with SEQ ID NO: 218; d) a variable heavy domain with SEQ ID NO: 227 and a variable light domain with SEQ ID NO: 228; e) a variable heavy domain with SEQ ID NO: 257 and a variable light domain with SEQ ID NO: 258; f) a variable heavy domain with SEQ ID NO: 267 and a variable light domain with SEQ ID NO: 268; g) a variable heavy domain with SEQ ID NO: 277 and a variable light domain with SEQ ID NO: 278; h) a variable heavy domain with SEQ ID NO: 287 and a variable light domain with SEQ ID NO: 288; i) a variable heavy domain with SEQ ID NO: 297 and a variable light domain with SEQ ID NO: 298; j) a variable heavy domain with SEQ ID NO: 307 and a variable light domain with SEQ ID NO: 308; k) a variable heavy domain with SEQ ID NO: 317 and a variable light domain with SEQ ID NO: 318; l) a variable heavy domain with SEQ ID NO: 337 and a variable light domain with SEQ ID NO: 338; m) a variable heavy domain with SEQ ID NO: 347 and a variable light domain with SEQ ID NO: 348; n) a variable heavy domain with SEQ ID NO: 437 and a variable light domain with SEQ ID NO: 438; o) a variable heavy domain with SEQ ID NO: 457 and a variable light domain with SEQ ID NO: 458; p) a variable heavy domain with SEQ ID NO: 467 and a variable light domain with SEQ ID NO: 468; q) a variable heavy domain with SEQ ID NO: 477 and a variable light domain with SEQ ID NO: 478; r) a variable heavy domain with SEQ ID NO: 497 and a variable light domain with SEQ ID NO: 498; s) a variable heavy domain with SEQ ID NO: 537 and a variable light domain with SEQ ID NO: 538; t) a variable heavy domain with SEQ ID NO: 547 and a variable light domain with SEQ ID NO: 548; u) a variable heavy domain with SEQ ID NO: 567 and a variable light domain with SEQ ID NO: 568; v) a variable heavy domain with SEQ ID NO: 597 and a variable light domain with SEQ ID NO: 598; w) a variable heavy domain with SEQ ID NO: 617 and a variable light domain with SEQ ID NO: 618; x) a variable heavy domain with SEQ ID NO: 637 and a variable light domain with SEQ ID NO: 638; y) a variable heavy domain with SEQ ID NO: 707 and a variable light domain with SEQ ID NO: 708; all as shown in U.S. Pat. No. 11,447,567, hereby incorporated by reference for the sequences. Additionally, any sequence included above from U.S. Pat. No. 11,447,567 can also be individually included or excluded.

In some embodiments, the trispecific constructs of the invention include at least one anti-FLT3 antigen binding domain, as described herein and in the Figures. Described herein is a plurality of means for binding the ECD of human FLT3.

i. Mesothelin (MSLN) Antigen Binding Domains

In some embodiments, the trispecific constructs include an ABD that binds to the ECD of human MSLN. Suitable variable heavy and variable light domains that bind to the ECD of human MSLN, but are not limited to, the sequences shown in FIGS. 156A-Z and 156AA-FF. It should be noted that these ABDs can be included in any of the formats described herein. Additionally, any sequence included above from FIG. 156 can also be individually included or excluded.

In some embodiments, an anti-human MSLN antigen binding domain includes a combination of VH and VL sequences, such as those shown in FIGS. 156EE and 156FF. Additionally, any sequence included above from FIG. 156 can also be individually included or excluded.

Included herein is an anti-human MSLN antigen binding domain with sequences selected from the group including: a) a VH with the amino acid sequence shown in SEQ ID NO:106 and a VL with the amino acid sequence shown in SEQ ID NO:106; b) a VH with the amino acid sequence shown in SEQ ID NO:114 and a VL with the amino acid sequence shown in SEQ ID NO:15; and c) a VH with the amino acid sequence shown in SEQ ID NO:122 and a VL with the amino acid sequence shown in SEQ ID NO:123; wherein the sequences are as shown in Table 4 of WO2019/234220, hereby expressly incorporated by reference for the sequences. Additionally, any sequence included above from Table 4 of WO2019/234220 can also be individually included or excluded. In some cases, other anti-human MSLN antigen binding domains are described in WO2018/157147. Additionally, any sequence included above from WO2018/157147 can also be individually included or excluded.

In some embodiments, the trispecific constructs of the invention include at least one anti-MSLN antigen binding domain, as described herein and in the Figures. Described herein is a plurality of means for binding the ECD of human MSLN.

j. Trop-2 Antigen Binding Domains

In some embodiments, the trispecific constructs include an ABD that binds to the ECD of human Trop2. Suitable variable heavy and variable light domains that bind to the ECD of human Trop2 include, but are not limited to, the sequences shown in FIGS. 157A-157R. Additionally, any sequence included above from FIG. 157 can also be individually included or excluded. It should be noted that these ABDs can be included in any of the formats described herein.

An anti-human Trop-2 antigen binding domain with VH and VL sequences selected from the group including: a) a VH having the amino acid sequence of positions 20 to 140 of SEQ ID NO:12 and a VL having the amino acid sequence of positions 21 to 129 of SEQ ID NO:18; b) a VH having the amino acid sequence of positions 20 to 140 of SEQ ID NO:14 and a VL having the amino acid sequence of positions 21 to 129 of SEQ ID NO:18; c) a VH having the amino acid sequence of positions 20 to 140 of SEQ ID NO:14 and a VL having the amino acid sequence of positions 21 to 129 of SEQ ID NO:20; d) a VH having the amino acid sequence of positions 20 to 140 of SEQ ID NO:16 and a VL having the amino acid sequence of positions 21 to 129 of SEQ ID NO:22; wherein the sequences are as shown in U.S. Pat. No. 9,850,312, hereby expressly incorporated by reference for the sequences. Additionally, any sequence included above from U.S. Pat. No. 9,850,312 can also be individually included or excluded.

An anti-human Trop-2 antigen binding domain with VH and VL sequences selected from the group including: a) a VH having the amino acid sequence of SEQ ID NO:172 and a VL having the amino acid sequence of SEQ ID NO:173; b) a VH having the amino acid sequence of SEQ ID NO:180 and a VL having the amino acid sequence of SEQ ID NO: 181; c) a VH having the amino acid sequence of SEQ ID NO:188 and a VL having the amino acid sequence of SEQ ID NO: 189; d) a VH having the amino acid sequence of SEQ ID NO:190 and a VL having the amino acid sequence of SEQ ID NO:191; and e) a VH having the amino acid sequence of SEQ ID NO:192 and a VL having the amino acid sequence of SEQ ID NO:193; wherein the sequences are as shown in Table 5 of WO2018/157147 hereby expressly incorporated by reference for the sequences. Additionally, any sequence included above from Table 5 of WO2018/157147 can also be individually included or excluded.

In some embodiments, the trispecific constructs of the invention include at least one anti-Trop2 antigen binding domain, as described herein and in the Figures. Described herein is plurality of a means for binding the ECD of human Trop2.

k. CEA Antigen Binding Domains

In some embodiments, the trispecific constructs include an ABD that binds to the ECD of human CEA. Suitable variable heavy and variable light domains that bind to the ECD of human CEA, but are not limited to, the sequences shown in FIGS. 158A-158C. Additionally, any sequence included above from FIG. 158 can also be individually included or excluded. It should be noted that these ABDs can be included in any of the formats described herein.

An anti-human CEA antigen binding domain with sequences selected from the group including: a) a VH with the amino acid sequence shown in SEQ ID NO:195 and a VL with the amino acid sequence shown in SEQ ID NO:196; b) a VH with the amino acid sequence shown in SEQ ID NO:203 and a VL with the amino acid sequence shown in SEQ ID NO:204; and c) a VH with the amino acid sequence shown in SEQ ID NO:211 and a VL with the amino acid sequence shown in SEQ ID NO:212; wherein the sequences are as shown in Table 6 WO2018/157147, hereby expressly incorporated by reference for the sequences. Additionally, any sequence included above from Table 6 of WO2018/157147 can also be individually included or excluded.

In some embodiments, the trispecific constructs of the invention include at least one anti-CEA antigen binding domain, as described herein and in the Figures. Described herein is a plurality of means for binding the ECD of human CEA.

l. CLDN18.2 Antigen Binding Domains

In some embodiments, the trispecific constructs include an ABD that binds to the ECD of human CLDN18.2. Suitable variable heavy and variable light domains that bind to the ECD of human CLDN18.2, but are not limited to, the sequences shown in FIG. 159. I Additionally, any sequence included above from FIG. 159 can also be individually included or excluded. t should be noted that these ABDs can be included in any of the formats described herein.

An anti-human CLDN18.2 antigen binding domain comprising a variable heavy domain and a variable light domain selected from the group including: a) a VH with the amino acid sequence depicted in SEQ ID NO:220 and a VL with the amino acid sequence depicted in SEQ ID NO:221; and b) a VL with the amino acid sequence depicted in SEQ ID NO:228 and a VL with the amino acid sequence depicted in SEQ ID NO:229, wherein the sequences are as depicted in WO2018/157147, hereby incorporated by reference for the sequences.

In some embodiments, the trispecific fusion proteins of the invention include at least one anti-CLDN18.2 antigen binding domain, as described herein and in the Figures. Described herein is a plurality of means for binding the ECD of human CLDN18.2.

m. BCMA Antigen Binding Domains

In some embodiments, the trispecific constructs include an ABD that binds to the ECD of human BCMA. Suitable variable heavy and variable light domains that bind to the ECD of human BCMA, but are not limited to, the sequences shown in FIGS. 160A-160C. Additionally, any sequence included above from FIG. 160 can also be individually included or excluded. It should be noted that these ABDs can be included in any of the formats described herein.

Provided herein is an anti-human BCMA antigen binding domain having a VH with the amino acid sequence depicted in SEQ ID NO:65 and a VL with the amino acid sequence depicted in SEQ ID NO: 66, wherein the sequences as are depicted in WO2019/198501, hereby incorporated by reference for these sequences. Additionally, any sequence included above from WO2019/198501 can also be individually included or excluded.

Also provided herein, is an anti-BCMA antigen binding domain having a variable heavy and a variable light domain with amino acid sequences selected from the sets comprising: a) a VH with SEQ ID NO:1118 and a VL with SEQ ID NO:1119; b) a VH with SEQ ID NO:1123 and a VL with SEQ ID NO:1124; c) a VH with SEQ ID NO:1127 and a VL with SEQ ID NO:1128; d) a VH with SEQ ID NO:1131 and a VL with SEQ ID NO:1132; e) a VH with SEQ ID NO: 1153 and a VL with SEQ ID NO:1154; f) a VH with SEQ ID NO:79 and a VL with SEQ ID NO:94; g) a VH with SEQ ID NO:69 and a VL with SEQ ID NO:84; h) a VH with SEQ ID NO: 70 and a VL with SEQ ID NO:85; i) a VH with SEQ ID NO:71 and a VL with SEQ ID NO:86; j) a VH with SEQ ID NO:72 and a VL with SEQ ID NO:87; k) a VH with SEQ ID NO: 73 and a VL with SEQ ID NO:88; l) a VH with SEQ ID NO:74 and a VL with SEQ ID NO:89; m) a VH with SEQ ID NO:75 and a VL with SEQ ID NO:90; n) a VH with SEQ ID NO: 76 and a VL with SEQ ID NO:91; o) a VH with SEQ ID NO77: and a VL with SEQ ID NO:92; p) a VH with SEQ ID NO:78 and a VL with SEQ ID NO:93; q) a VH with SEQ ID NO:80 and a VL with SEQ ID NO:95; r) a VH with SEQ ID NO:81 and a VL with SEQ ID NO:96; s) a VH with SEQ ID NO:82 and a VL with SEQ ID NO:97; t) a VH with SEQ ID NO:83 and a VL with SEQ ID NO:98; u) a VH with SEQ ID NO:171 and a VL with SEQ ID NO:192; v) a VH with SEQ ID NO:172 and a VL with SEQ ID NO:193; w) a VH with SEQ ID NO: 173 and a VL with SEQ ID NO:194; x) a VH with SEQ ID NO:174 and a VL with SEQ ID NO:195; y) a VH with SEQ ID NO:175 and a VL with SEQ ID NO:196; z) a VH with SEQ ID NO: 176 and a VL with SEQ ID NO:197; aa) a VH with SEQ ID NO: 177 and a VL with SEQ ID NO:198; bb) a VH with SEQ ID NO: 178 and a VL with SEQ ID NO:199; cc) a VH with SEQ ID NO:179 and a VL with SEQ ID NO:200; dd) a VH with SEQ ID NO: 180 and a VL with SEQ ID NO:201; ee) a VH with SEQ ID NO:181 and a VL with SEQ ID NO:202; ff) a VH with SEQ ID NO:182 and a VL with SEQ ID NO:203; gg) a VH with SEQ ID NO: 183 and a VL with SEQ ID NO:204; hh) a VH with SEQ ID NO:184 and a VL with SEQ ID NO:205; ii) a VH with SEQ ID NO:185 and a VL with SEQ ID NO:206; jj) a VH with SEQ ID NO:186 and a VL with SEQ ID NO:207; kk) a VH with SEQ ID NO:187 and a VL with SEQ ID NO:208; ll) a VH with SEQ ID NO:188 and a VL with SEQ ID NO:209; mm) a VH with SEQ ID NO:189 and a VL with SEQ ID NO:210; nn) a VH with SEQ ID NO:190 and a VL with SEQ ID NO:211; and oo) a VH with SEQ ID NO:191 and a VL with SEQ ID NO:212; wherein the sequences as are depicted in US2020/0179511, hereby incorporated by reference for these sequences. Additionally, any sequence included above from US2020/0179511 can also be individually included or excluded.

In some embodiments, the trispecific constructs of the invention include at least one anti-BCMA antigen binding domain, as described herein and in the Figures. Described herein is a plurality of means for binding the ECD of human BCMA.

n. PD-1 Antigen Binding Domains

In some embodiments, the trispecific constructs include an ABD that binds to the ECD of human PD-1. Suitable variable heavy and variable light domains that bind to the ECD of human PD-1, but are not limited to, the sequences shown in FIGS. 161A-161G. Additionally, any sequence included above from FIG. 161 can also be individually included or excluded. It should be noted that these ABDs can be included in any of the formats described herein.

In some embodiments, the trispecific constructs of the invention include at least one anti-PD-1 antigen binding domain, as described herein and in the Figures. Described herein is a plurality of means for binding the ECD of human PD-1.

o. ANO1 Antigen Binding Domains

In some embodiments, the TTA binds to the ECD of human ANO1. In this case, the amino acid sequence of the variable heavy domain is shown in SEQ ID NO:97 of Table 3 of WO2018/157147 and the sequence of the variable light domain is shown in SEQ ID NO:98, the sequences of which are hereby incorporated by reference.

In some embodiments, the trispecific constructs of the invention include at least one anti-ANO1 antigen binding domain, as described herein and in the Figures. Described herein is a means for binding the ECD of human ANO1 antigen.

p. CD22 Antigen Binding Domains

In some embodiments, the TTA binds to the ECD of human CD22. In some embodiments, the amino acid sequence of the variable heavy domain is shown in SEQ ID NO:30 and the variable light domain is shown in SEQ ID NO:28 of U.S. Pat. No. 7,355,011, the sequences of which are hereby incorporated by reference. In some embodiments, the CD22 ABD comprises the variable heavy and variable light domains of an antibody selected from the group including: epratuzumab or moxetumomab.

In some embodiments, the trispecific constructs of the invention include at least one anti-CD22 antigen binding domain, as described herein and in the Figures. Described herein is a means for binding the ECD of human CD22 antigen.

q. CD38 Antigen Binding Domains

In some embodiments, the TTA binds to the ECD of human CD38. In some embodiments, the CD22 ABD comprises the variable heavy and variable light domains of an antibody selected from the group including: daratumumab, isatuximab, felzartamab or mezagitamab.

In some embodiments, the trispecific constructs of the invention include at least one anti-CD38 antigen binding domain, as described herein and in the Figures. Described herein is a means for binding the ECD of human CD38 antigen.

### XI. IL18-Fc Fusion Protein Formats

Useful IL18-Fc fusion protein formats are shown in FIG. 12. IL18-Fc fusion proteins provided herein include monovalent IL18-Fc fusion proteins as shown in FIGS. 12A and 21A-21P) and IL18×Fab-Fc fusion proteins as shown in FIGS. 12B and 22.

1. IL18 Heterodimeric Fc Fusion Proteins

In some embodiments, the IL18-Fc fusion is monovalent IL18-Fc fusion protein that includes (a) a first monomer that includes an TL18 protein or a variant thereof covalently attached to a first Fc domain, and (b) a second monomer that includes a second Fc domain alone (i.e., an “empty Fc”). In some embodiments, the IL18 fusion is monovalent IL18-Fc fusion protein includes (a) a monomer that includes an TL18 covalently attached to an Fc domain and (b) another monomer that includes another Fc domain alone (i.e., an “empty Fc”). See, the schematic diagram in FIG. 12A and the amino acid sequences of FIGS. 21A-21I.

Any of the IL18s described herein can be included in the monovalent IL18-Fc fusion protein. In some embodiments, the IL18 is wildtype mature human IL18 (FIG. 1A). In certain embodiments, the IL18 is a variant IL18 that includes one or more modifications as depicted in FIGS. 13A-13B, 14, 15A-15B, 16A-16C, 17, 18, 19A-19I, 20A-20C, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 of the monovalent IL18-Fc fusion proteins is a variant IL18 that includes a modification at one or more positions selected from Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, 171, C76, E77, 180, I81, N87, P88, D90, K93, T95, K96, S97, Q103, H109, D110, N111, M113, S119, A126, C127, D132, L136, L138, K139, E141, L144, D146, R147, 1149, M150, V153, N155, E156, and D157 are modified. In some embodiments, the IL18 of the monovalent IL18-Fc fusion proteins is a variant IL18 that includes one or more modifications (e.g., substitutions or deletions) selected from Y1F, Y1H, E6A, E6Q, S7C, S7P, K8E, K8Q, K8Y, K8R, K8D, K8N, K8S, K8T, S10C, V11, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, I49D, I49E, I49N, I49Q, I49Y, I49F, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, M60R, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, Q103Y, Q103E, Q103K, Q103R, H109W, H109Y, D110N, N111D, D110Q, D110R, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, 1149V, M150F, M150T, V153E, V153K, V153R, V153Y, V153Q, V153N, V153D, N155C, E156Q, D157A, D157S, D157N, and D157del. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C, M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the IL18-Fc fusion protein includes one or more amino acid substitutions provided in FIGS. 13A-13B, 14, 15A-15B, 16A-16C, 17, 18, 19A-191, 20A-20C, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant of the IL18-Fc fusion protein is depicted in any one of FIGS. 13A-13B, 14, 15A-15B, 16A-16C, 17, 18, 19A-191, 20A-20C, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the monovalent IL18-Fc fusion protein, including the wildtype and variant Fc domains described herein. In some embodiments, each Fc domain includes a CH2 and CH3. In some embodiments, the first and second Fc domains include a hinge, CH2 and CH3. In one embodiment, the first and second Fc domains each have the formula, from N-terminus to C-terminus, hinge-CH2-CH3. In some embodiments, the first and second Fc domains of the monovalent IL18-Fc fusion protein are heterodimeric. Modifications for such Fc domains are described in Sections above.

In an exemplary embodiment, the monovalent IL18-Fc fusion protein is heterodimeric. In some embodiments, the first and second Fc domains include the amino acid substitution set L368D/K370S:S364K/E357Q. In some embodiments, the L368D/K370S modifications are in the first Fc domain and the S364K/E357Q modifications are in the second domain. In certain heterodimeric embodiments, the first Fe domain includes isosteric pI variants Q295E/N384D/Q418E/N421D. In some embodiments, the first Fe domain and the second Fc domain each include K447del modifications. In some embodiments, a IL18 protein or variant thereof is connected to the first Fc domain.

In certain embodiments, both the first and second Fc domains include FcKO variants:E 233P/L234V/L235A/G236del/S267K, according to the EU numbering.

In some embodiments, the first monomer includes a first Fc domain with heterodimer skew variants L368D/K370S, isosteric pI variants Q295E/N384D/Q418E/N421D, and FcKO variants E233P/L234V/L235A/G236del/S267K and the second monomer includes a second Fc domain with heterodimer skew variants S364K/E357Q and FcKO variants E233P/L234V/L235A/G236del/S267K, according to the EU index. In some embodiments, the first and second monomers each also include M428L/N434S half-life extension variants. In some embodiments, a IL18 protein or variant thereof is connected to the first Fc domain.

In some embodiments, the first and second monomers each also include a C220S hinge amino acid substitution. In some embodiments, the first and second monomers each also include a N297A or N297S amino acid substitution that removes glycosylation. In some embodiments, the first monomer includes a first Fc domain with modifications C220S/E233P/L234V/L235A/G236del/S267K/Q295E/L368D/K370S/384D/Q418E/N421D and optionally M428L/N434S and the second monomer includes a second Fc domain with modifications C220S/E233P/L234V/L235A/G236del/S267K/S364K/E357Q and optionally modifications M428L/N434S, according to the EU numbering. In some embodiments, a IL18 protein or variant thereof is connected to the first Fc domain.

FIGS. 21A-21I depict amino acid modifications in the first and second monomers of a heterodimeric monovalent IL18-Fc fusion protein. Additional, exemplary Fc domain “backbone sequences” that find use in the subject monovalent IL18-Fc fusion proteins are depicted in FIGS. 9A-9E and 10.

In the formulas above, “IL18” is any IL18 provided herein (see, e.g., wildtype or variant IL18 depicted in FIGS. 13A-13B, 14, 15A-15B, 16A-16C, 17, 18, 19A-19I, 20A-20C, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

Exemplary monovalent IL18 fusion proteins include, XENP30792 (SEQ ID NOS: 239-240), XENP31296 (SEQ ID NOS: 241-242), XENP39804 (SEQ ID NOS: 285-286), XENP40685 (SEQ ID NOS: 960-961), XENP40962 (SEQ ID NOS: 962-963), XENP40963 (SEQ ID NOS: 964-965), XENP40964 (SEQ ID NOS: 966-967), XENP40965 (SEQ ID NOS: 968-969), XENP40966 (SEQ ID NOS: 970-971), XENP40967 (SEQ ID NOS: 972-973), XENP40968 (SEQ ID NOS: 974-975), XENP41756 (SEQ ID NOS: 976-977), XENP41758 (SEQ ID NOS: 980-981), XENP41760 (SEQ ID NOS: 984-985), XENP41761 (SEQ ID NOS: 986-987), XENP41762 (SEQ ID NOS: 988-989), XENP41763 (SEQ ID NOS: 990-991), XENP41770 (SEQ ID NOS: 1004-1005), XENP41974 (SEQ ID NOS: 1006-1007), and XENP42006 (SEQ ID NOS: 1010-1011), XENP42007 (SEQ ID NOS: 1012-1013), XENP42141 (SEQ ID NOS: 1024-1025), XENP42143 (SEQ ID NOS: 1028-1029), XENP42145 (SEQ ID NOS: 1032-1033) and as shown in FIGS. 21A-21I and the sequence listing.

2. IL18× Fab Heterodimeric Fc Fusion Proteins

In some embodiments, the IL18 fusion is IL18× Fab-Fc fusion protein that includes (a) a first monomer comprising a variable heavy (VH) region covalently attached to the N-terminus of a first heterodimeric Fc chain, (b) a second monomer comprising an IL18 protein or variant thereof covalently attached to the N-terminus of a complementary second heterodimeric Fc chain (optionally via a domain linker), and (c) a third monomer that is a corresponding light chain that forms a Fab with the first monomer. The Fc chain of the first monomer and the Fc chain of the second monomer form a heterodimeric Fc complex. See, the schematic diagram in FIG. 12B and the amino acid sequences of FIGS. 22A-22Z, 22AA-AZ, 22BA-22BZ, 22CA-22CZ, and 22DA-22DO.

Any of the IL18s described herein can be included in the monovalent IL18-Fc fusion protein. In some embodiments, the IL18 is wildtype mature human IL18 (FIG. 1A). In certain embodiments, the IL18 is a variant IL18 that includes one or more modifications as depicted in FIGS. 13A-13B, 14, 15A-15B, 16A-16C, 17, 18, 19A-19I, 20A-20C, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, one or more residues of IL18 selected from Y1, E6, S7, K8, S10, V11, N14, L15, D17, Q18, D23, R27, P28, L29, E31, M33, T34, D35, S36, D37, C38, R39, D40, N41, R44, I46, I49, 550, M51, K53, D54, S55, Q56, P57, M60, A61, V62, T63, S65, K67, C68, E69, 171, C76, E77, 180, 181, N87, P88, D90, K93, T95, K96, S97, Q103, H109, D110, N111, M113, S119, A126, C127, D132, L136, L138, K139, E141, L144, D146, R147, 1149, M150, V153, N155, E156, and D157 are modified. In some embodiments, the IL18 of the IL18× Fab-Fc fusion proteins is a variant IL18 that includes one or more modifications (e.g., substitutions or deletions) selected from Y1F, Y1H, E6A, E6Q, S7C, S7P, K8E, K8Q, K8Y, K8R, K8D, K8N, K8S, K8T, S10C, V11, N14C, N14W, L15C, D17N, Q18L, D23N, D23S, R27Q, P28C, L29V, E3IQ, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, I49D, I49E, I49N, I49Q, I49Y, I49F, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, M60R, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, Q103Y, Q103E, Q103K, Q103R, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, 1149V, M150F, M150T, V153E, V153K, V153R, V153Y, V153Q, V153N, V153D, N155C, E156Q, D157A, D157S, D157N, and D157del. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C, M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the IL18× Fab-Fc fusion protein includes one or more amino acid substitutions provided in FIGS. 13A-13B, 14, 15A-15B, 16A-16C, 17, 18, 19A-19I, 20A-20C, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15B, 16A-16C, 17, 18, 19A-191, 20A-20C, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368, and the sequence listing.

Any Fc domains can be included in the IL18× Fab-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the IL18-Fc fusion protein are heterodimeric. Modifications for such Fc domains are described in Sections above.

In exemplary embodiments, the IL18× Fab-Fc fusion protein is a heterodimeric Fc fusion protein. In some heterodimeric embodiments, the first and second Fc domains include the amino acid substitution set L368D/K370S:S364K/E357Q. In some embodiments, the S364K/E357Q modifications are in the first Fc domain and the L368D/K370S modifications are in the second Fc domain. In certain heterodimeric embodiments, the second Fc domain includes isosteric pI variants Q295E/N384D/Q418E/N421D. In certain embodiments, both the first and second Fc domains include FcKO variants:E233P/L234V/L235A/G236del/S267K, according to the EU numbering. In some embodiments, the first Fc domain and the second Fc domain each include K447del modifications. In some embodiments, the IL18 protein or variant thereof is linked to the Fc domain that includes isosteric pI variants (e.g., the second Fc domain).

In exemplary embodiments, the IL18× Fab-Fc fusion protein is a heterodimeric Fc fusion protein containing a first monomer, a second monomer and a third monomer. In some embodiments, the first monomer includes a variable heavy chain, the second monomer includes an IL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIGS. 21A-21P depict amino acid modifications in the first and second monomers of a heterodimeric monovalent IL18-Fc fusion protein. Additional, exemplary Fc domain “backbone sequences” that find use in the subject monovalent IL18-Fc fusion proteins are depicted in FIGS. 9A-9D and 10.

In the formulas above, “IL18” is any IL18 provided herein (see, e.g., wildtype or variant IL18 depicted in FIGS. 13A-13B, 14, 15A-15B, 16A-16C, 17, 18, 19A-19I, 20A-20C, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

Exemplary IL18×Fab-Fc fusion protein formats include XENP37827, XENP38850, XENP38851, XENP38852, XENP38853, XENP38854, XENP38855, XENP38856, XENP38858, XENP38859, XENP38860, XENP38861, XENP38861, XENP38862, XENP38863, XENP38864, XENP38865, XENP38866, XENP38867, XENP38868, XENP40027, XENP40046, XENP40047, XENP40048, XENP40049, XENP40050, XENP40051, XENP40052, XENP40053, XENP40054, XENP40175, XENP40176, XENP40177, XENP40178, XENP40179, XENP40180, XENP40181, XENP40182, XENP40183, XENP40184, XENP40185, XENP40186, XENP40187, XENP40188, XENP40189, XENP40190, XENP40190, XENP40191, XENP40192, XENP40193, XENP40194, XENP40195, XENP40196, XENP40197, XENP40198, XENP40199, XENP40200, XENP40201, XENP40202, XENP40203, XENP40204, XENP40205, XENP40206, XENP40207, XENP40208, XENP40209, XENP40210, XENP40211, XENP40212, XENP40213, XENP40214, XENP40215, XENP40216, XENP40217, XENP40218, XENP40219, XENP40220, XENP40221, XENP40222, XENP40223, XENP40224, XENP40225, XENP40226, XENP40227, XENP40228, XENP40229, XENP40230, XENP40231, XENP40232, XENP40233, XENP40234, XENP40235, XENP40236, XENP40237, XENP40238, XENP40239, XENP40240, XENP40241, XENP40242, XENP40243, XENP40244, XENP40246, XENP40247, XENP40248, XENP40249, XENP40250, XENP40251, XENP40252, XENP40253, XENP40254, XENP40255, XENP40256, XENP40257, XENP40258, XENP40259, XENP40260, XENP40261, XENP40262, XENP40263, XENP40264, XENP402655, XENP40266, XENP40267, XENP40268, XENP40269, XENP40617, XENP40618, XENP40619, XENP40620, XENP40621, XENP40622, XENP40623, XENP40624, XENP40625, XENP40626, XENP40627, XENP40628, XENP40629, XENP40630, XENP40630, XENP40631, XENP40632, XENP40657, XENP40658, XENP40659, XENP40660, XENP40661, XENP40662, XENP40663, XENP40686, XENP40934, XENP40935, XENP40936, XENP40937, XENP40938, XENP40939, XENP40940, XENP40941, XENP40942, XENP40943, XENP40944, XENP40945, XENP40946, XENP40947, XENP40948, XENP40949, XENP40950, XENP40951, XENP40952, XENP40953, XENP40954, XENP40955, XENP40956, XENP40957, XENP40958, XENP40959, XENP40960, XENP40961, XENP41076, XENP41091, XENP41092, XENP41353, XENP41416, XENP41417, XENP41418, XENP41419, XENP41420, XENP41421, XENP41422, XENP41423, XENP41424, XENP41425, XENP414260, XENP41427, XENP41428, XENP41429, XENP41430, XENP41431, XENP41440, XENP44149, XENP44150, XENP44151, XENP44152, XENP44153, XENP44154, XENP44155, XENP44156, XENP44157, XENP44158, XENP44159, XENP44160, XENP44161, XENP44162, XENP44163, XENP44164, XENP44165, XENP44166, XENP44167, XENP44168, XENP44169, XENP44170, XENP44172, XENP44173, XENP44174, XENP44687, XENP44688, XENP44689, XENP44690, XENP44691, XENP44692, XENP44693, XENP44694, XENP44695, XENP44741, XENP44742, XENP44743, XENP44744, XENP44745, XENP44741, XENP44746, and XENP41095, as shown in FIGS. 22A-22DO and those provided in the sequence listing, such as the constructs (chains 1, 2 and 3) depicted in SEQ ID NOS: 118-237, 249-254, 293-664, 667-774, 1040-1241, for example, SEQ ID NOS:781, 665 and 666 as chains 1, 2, 3, respectively.

3. IL18 Trispecific Heterodimeric Fc Fusion Proteins

a. 1+1+1 Fab-scFv-Fc×IL18-Fc

In some embodiments, the IL18 fusion is 1+1+1 Fab-scFv-Fc×IL18-Fc that includes (a) a first monomer comprising from N-terminus to C-terminus: a first variable heavy domain (VH1), an scFv, and a first Fc domain; (b) a second monomer comprising from N-terminus to C-terminus: an IL-18 protein and a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen. The Fc chain of the first monomer and the Fc chain of the second monomer form a heterodimeric Fc complex. See, the schematic diagram in FIG. 118A and the amino acid sequences of FIG. 123.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 1+1+1 Fab-scFv-Fc×IL18-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C, M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the 1+1+1 Fab-scFv-Fc×IL18-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368, and the sequence listing.

Any Fc domains can be included in the 1+1+1 Fab-scFv-Fc×IL18-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fe domains of the monovalent IL18-Fc fusion protein are heterodimeric. Modifications for such Fe domains are described in Sections above.

In exemplary embodiments, the 1+1+1 Fab-scFv-Fc×IL18-Fc fusion protein is a heterodimeric Fc fusion protein. In some heterodimeric embodiments, the first and second Fe domains include the amino acid substitution set L368D/K370S. S364K/E357Q. In some embodiments, the S364K/E357Q modifications are in the first Fe domain and the L368D/K370S modifications are in the second Fe domain. In some embodiments, the S364K/E357Q modifications are in the second Fe domain and the L368D/K370S modifications are in the first Fe domain. In certain heterodimeric embodiments, the first or second Fe domain includes isosteric pI variants Q295E/N384D/Q418E/N421D. In certain embodiments, both the first and second Fe domains include FcKO variants:E233P/L234V/L235A/G236del/S267K, according to the EU numbering. In some embodiments, the first Fe domain and the second Fe domain each include K447del modifications. In some embodiments, the IL18 protein or variant thereof is linked to the Fe domain that includes isosteric pI variants (e.g., the second Fe domain).

In exemplary embodiments, the 1+1+1 Fab-scFv-Fc×IL18-Fc fusion protein is a heterodimeric Fc fusion protein containing a first monomer, a second monomer and a third monomer. In some embodiments, the first monomer includes a variable heavy chain, the second monomer includes an TL18 protein or variant thereof, and the third monomer includes a variable light chain. In some embodiments, the first monomer includes a first Fe domain with heterodimer skew variants S364K/E357Q and FcKO variants E233P/L234V/L235A/G236del/S267K, according to the EU index. In some embodiments, the second monomer includes a second Fe domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fe domain with modifications C220S/E233P/L234V/L235A/G236del/S267K/S364K/E357Q and optionally modifications M428L/N434S, the second monomer includes a first Fe domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIG. 123 depict amino acid modifications in the first and second monomers of a heterodimeric IL18-Fc fusion protein. Additional, exemplary Fc domain “backbone sequences” that find use in the subject 1+1+1 Fab-scFv-Fc×IL18-Fc are depicted in FIGS. 9A-9E and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

b. 1+1+1 scFv-Fc×IL18-Fab-Fc

In some embodiments, the IL18 fusion is 1+1+1 scFv-Fc×IL18-Fab-Fc that includes (a) a first monomer comprising from N-terminus to C-terminus: an scFv and a first Fc domain; (b) a second monomer comprising from N-terminus to C-terminus: an IL18 protein, a first variable heavy domain (VH1), and a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen. The Fc chain of the first monomer and the Fc chain of the second monomer form a heterodimeric Fc complex. See, the schematic diagram in FIG. 118B and the amino acid sequences of FIG. 124.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 1+1+1 scFv-Fc×IL18-Fab-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the 1+1+1 scFv-Fc×IL18-Fab-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the 1+1+1 scFv-Fc×IL18-Fab-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections above.

In exemplary embodiments, the 1+1+1 scFv-Fc×IL18-Fab-Fc fusion protein is a heterodimeric Fc fusion protein. In some heterodimeric embodiments, the first and second Fc domains include the amino acid substitution set L368D/K370S:S364K/E357Q. In some embodiments, the S364K/E357Q modifications are in the first Fc domain and the L368D/K370S modifications are in the second Fc domain. In some embodiments, the S364K/E357Q modifications are in the second Fc domain and the L368D/K370S modifications are in the first Fc domain. In certain heterodimeric embodiments, the first or second Fc domain includes isosteric pI variants Q295E/N384D/Q418E/N421D. In certain embodiments, both the first and second Fc domains include FcKO variants:E233P/L234V/L235A/G236del/S267K, according to the EU numbering. In some embodiments, the first Fc domain and the second Fc domain each include K447del modifications. In some embodiments, the IL18 protein or variant thereof is linked to the Fc domain that includes isosteric pI variants (e.g., the second Fc domain).

In exemplary embodiments, the 1+1+1 scFv-Fc×IL18-Fab-Fc fusion protein is a heterodimeric Fc fusion protein containing a first monomer, a second monomer and a third monomer. In some embodiments, the first monomer includes a variable heavy chain, the second monomer includes an IL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIG. 124 depict amino acid modifications in the first and second monomers of a heterodimeric IL18-Fc fusion protein. Additional, exemplary Fc domain “backbone sequences” that find use in the subject 1+1+1 scFv-Fc×IL18-Fab-Fc are depicted in FIGS. 9A-9E and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

c. 1+1+1 IL18-scFv-Fc× Fab-Fc

In some embodiments, the IL18 fusion is 1+1+1 IL18-scFv-Fc× Fab-Fc that includes (a) a first monomer comprising from N-terminus to C-terminus: an IL18 protein, an scFv, and a first Fe domain; (b) a second monomer comprising from N-terminus to C-terminus: a first variable heavy domain (VH1) and a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen. The Fc chain of the first monomer and the Fc chain of the second monomer form a heterodimeric Fc complex. See, the schematic diagram in FIG. 118C and the amino acid sequences of FIG. 125.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 1+1+1 IL18-scFv-Fc× Fab-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111S/M113K, M51K/K53T/Q56R/P57A/Q103E/S105D/D110N/N111D/M113R, M51R/Q56G/P57K/M60L/Q103R/D110 S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the 1+1+1 IL18-scFv-Fc× Fab-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the 1+1+1 IL18-scFv-Fc× Fab-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections above.

In exemplary embodiments, the 1+1+1 IL18-scFv-Fc× Fab-Fc fusion protein is a heterodimeric Fc fusion protein. In some heterodimeric embodiments, the first and second Fc domains include the amino acid substitution set L368D/K370S:S364K/E357Q. In some embodiments, the S364K/E357Q modifications are in the first Fc domain and the L368D/K370S modifications are in the second Fc domain. In some embodiments, the S364K/E357Q modifications are in the second Fc domain and the L368D/K370S modifications are in the first Fc domain. In certain heterodimeric embodiments, the first or second Fc domain includes isosteric pI variants Q295E/N384D/Q418E/N421D. In certain embodiments, both the first and second Fc domains include FcKO variants:E233P/L234V/L235A/G236del/S267K, according to the EU numbering. In some embodiments, the first Fe domain and the second Fe domain each include K447del modifications. In some embodiments, the IL18 protein or variant thereof is linked to the Fc domain that includes isosteric pI variants (e.g., the second Fc domain).

In exemplary embodiments, the 1+1+1 IL18-scFv-Fc× Fab-Fc fusion protein is a heterodimeric Fc fusion protein containing a first monomer, a second monomer and a third monomer. In some embodiments, the first monomer includes a variable heavy chain, the second monomer includes an TL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIG. 125 depict amino acid modifications in the first and second monomers of a heterodimeric IL18-Fc fusion protein. Additional, exemplary Fc domain “backbone sequences” that find use in the subject 1+1+1 IL18-scFv-Fc× Fab-Fc are depicted in FIGS. 9A-9D and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fe domain provided herein (e.g., wildtype or variant Fe domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fe domain (i.e., an “empty Fe domain”). In some embodiments, the each of the first and second Fe domains have the formula N-hinge-CH2-CH3-C. In certain embodiments, each of the first and second Fe domains have the formula N-CH2-CH3-C.

d. 1+1+1 Fab-Fc-scFv×IL18-Fc

In some embodiments, the IL18 fusion is 1+1+1 Fab-Fc-scFv×IL18-Fc that includes (a) a first monomer comprising from N-terminus to C-terminus: a first variable heavy domain (VH1), a first Fe domain, and an scFv; (b) a second monomer comprising from N-terminus to C-terminus: an IL-18 protein and a second Fe domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen. The Fc chain of the first monomer and the Fc chain of the second monomer form a heterodimeric Fc complex. See, the schematic diagram in FIG. 118D and the amino acid sequences of FIGS. 126 and 127.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 1+1+1 Fab-Fc-scFv×IL18-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111 Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the 1+1+1 Fab-Fc-scFv×IL18-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the 1+1+1 Fab-Fc-scFv×IL18-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections above.

In exemplary embodiments, the 1+1+1 Fab-Fc-scFv×IL18-Fc fusion protein is a heterodimeric Fc fusion protein. In some heterodimeric embodiments, the first and second Fe domains include the amino acid substitution set L368D/K370S. S364K/E357Q. In some embodiments, the S364K/E357Q modifications are in the first Fc domain and the L368D/K370S modifications are in the second Fc domain. In some embodiments, the S364K/E357Q modifications are in the second Fc domain and the L368D/K370S modifications are in the first Fc domain. In certain heterodimeric embodiments, the first or second Fc domain includes isosteric pI variants Q295E/N384D/Q418E/N421D. In certain embodiments, both the first and second Fc domains include FcKO variants:E233P/L234V/L235A/G236del/S267K, according to the EU numbering. In some embodiments, the first Fc domain and the second Fc domain each include K447del modifications. In some embodiments, the IL18 protein or variant thereof is linked to the Fc domain that includes isosteric pI variants (e.g., the second Fc domain).

In exemplary embodiments, the 1+1+1 Fab-Fc-scFv×IL18-Fc fusion protein is a heterodimeric Fc fusion protein containing a first monomer, a second monomer and a third monomer. In some embodiments, the first monomer includes a variable heavy chain, the second monomer includes an TL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220S/E233P/L234V/L235A/G236del/S267K/Q295E/L368D/K370S/384D/Q418E/N421D and optionally M428L/N434S, and according to the EU numbering and a third monomer that does not include an Fe domain. and according to the EU numbering. In some embodiments, the first Fc domain of the first monomer and the second Fc domain of the second monomer each include K447del modifications.

FIGS. 126 and 127 depict amino acid modifications in the first and second monomers of a heterodimeric IL18-Fc fusion protein. Additional, exemplary Fc domain “backbone sequences” that find use in the subject 1+1+1 Fab-Fc-scFv×IL18-Fc are depicted in FIGS. 9A-9E and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

e. 2+1+1 Fab-scFv-Fc×IL18-Fab-Fc

In some embodiments, the IL18 fusion is 2+1+1 Fab-scFv-Fc×IL18-Fab-Fc that includes (a) a first monomer comprising from N-terminus to C-terminus: a first variable heavy domain (VH1), an scFv, and a first Fc domain; (b) a second monomer comprising from N-terminus to C-terminus: an IL-18 protein, a second variable heavy domain (VH2), and a second Fc domain; and (c) a third monomer comprising from N-terminus to C-terminus: a first variable light domain (VL1) and a constant light domain, wherein the VH1 and the VL1 form a first antigen binding domain (ABD) to a first antigen, and wherein the VH2 and the VL1 form a second ABD to the first antigen, and wherein the scFv comprises a third variable heavy domain (VH3), a scFv linker, and a second variable light domain (VL2), wherein the VH3 and the VL2 together form a third ABD to a second antigen. The Fc chain of the first monomer and the Fc chain of the second monomer form a heterodimeric Fc complex. See, the schematic diagram in FIG. 118E and the amino acid sequences of FIG. 128.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 2+1+1 Fab-scFv-Fc×IL18-Fab-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110 S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V I/C38A/K53A/T63A/C76A/C127A.

In some embodiments, the IL18 variant of the 2+1+1 Fab-scFv-Fc×IL18-Fab-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the 2+1+1 Fab-scFv-Fc×IL18-Fab-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections above.

In exemplary embodiments, the 2+1+1 Fab-scFv-Fc×IL18-Fab-Fc fusion protein is a heterodimeric Fc fusion protein. In some heterodimeric embodiments, the first and second Fc domains include the amino acid substitution set L368D/K370S:S364K/E357Q. In some embodiments, the S364K/E357Q modifications are in the first Fc domain and the L368D/K370S modifications are in the second Fc domain. In some embodiments, the S364K/E357Q modifications are in the second Fc domain and the L368D/K370S modifications are in the first Fc domain. In certain heterodimeric embodiments, the first or second Fc domain includes isosteric pI variants Q295E/N384D/Q418E/N421D. In certain embodiments, both the first and second Fc domains include FcKO variants:E233P/L234V/L235A/G236del/S267K, according to the EU numbering. In some embodiments, the first Fc domain and the second Fc domain each include K447del modifications. In some embodiments, the IL18 protein or variant thereof is linked to the Fc domain that includes isosteric pI variants (e.g., the second Fc domain).

In exemplary embodiments, the 2+1+1 Fab-scFv-Fc×IL18-Fab-Fc fusion protein is a heterodimeric Fc fusion protein containing a first monomer, a second monomer and a third monomer. In some embodiments, the first monomer includes a variable heavy chain, the second monomer includes an TL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIG. 128 depict amino acid modifications in the first and second monomers of a heterodimeric IL18-Fc fusion protein. Additional, exemplary Fc domain “backbone sequences” that find use in the subject 2+1+1 Fab-scFv-Fc×IL18-Fab-Fc are depicted in FIGS. 9A-9E and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

f. 2+1+1 Fab-Fab-Fc×IL18-scFv-Fc

In some embodiments, the IL18 fusion is 2+1+1 Fab-Fab-Fc×IL18-scFv-Fc that includes (a) a first monomer comprising from N-terminus to C-terminus: an IL-18 protein, an scFv, and a first Fc domain; (b) a second monomer comprising from N-terminus to C-terminus: a first variable heavy domain (VH1), a second variable heavy domain (VH2), and a second Fe domain; and (c) a third monomer comprising from N-terminus to C-terminus: a first variable light domain (VL1) and a constant light domain, wherein the VH1 and the VL1 form a first antigen binding domain (ABD) to a first antigen, and wherein the VH2 and the VL1 form a second ABD to the first antigen, and wherein the scFv comprises a third variable heavy domain (VH3), a scFv linker, and a second variable light domain (VL2), wherein the VH3 and the VL2 together form a third ABD to a second antigen. The Fc chain of the first monomer and the Fc chain of the second monomer form a heterodimeric Fc complex. See, the schematic diagram in FIG. 118F and the amino acid sequences of FIG. 129.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 2+1+1 Fab-Fab-Fc×IL18-scFv-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110 S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the 2+1+1 Fab-Fab-Fc×IL18-scFv-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the 2+1+1 Fab-Fab-Fc×IL18-scFv-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections above.

In exemplary embodiments, the 2+1+1 Fab-Fab-Fc×IL18-scFv-Fc fusion protein is a heterodimeric Fc fusion protein. In some heterodimeric embodiments, the first and second Fc domains include the amino acid substitution set L368D/K370S:S364K/E357Q. In some embodiments, the S364K/E357Q modifications are in the first Fc domain and the L368D/K370S modifications are in the second Fc domain. In some embodiments, the S364K/E357Q modifications are in the second Fc domain and the L368D/K370S modifications are in the first Fc domain. In certain heterodimeric embodiments, the first or second Fc domain includes isosteric pI variants Q295E/N384D/Q418E/N421D. In certain embodiments, both the first and second Fc domains include FcKO variants:E233P/L234V/L235A/G236del/S267K, according to the EU numbering. In some embodiments, the first Fc domain and the second Fc domain each include K447del modifications. In some embodiments, the IL18 protein or variant thereof is linked to the Fc domain that includes isosteric pI variants (e.g., the second Fc domain).

In exemplary embodiments, the 2+1+1 Fab-Fab-Fc×IL18-scFv-Fc fusion protein is a heterodimeric Fc fusion protein containing a first monomer, a second monomer and a third monomer. In some embodiments, the first monomer includes a variable heavy chain, the second monomer includes an TL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIG. 129 depict amino acid modifications in the first and second monomers of a heterodimeric IL18-Fc fusion protein. Additional, exemplary Fc domain “backbone sequences” that find use in the subject 2+1+1 Fab-Fab-Fc×IL18-scFv-Fc are depicted in FIGS. 9A-9E and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fe domain provided herein (e.g., wildtype or variant Fe domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fe domain (i.e., an “empty Fe domain”). In some embodiments, the each of the first and second Fe domains have the formula N-hinge-CH2-CH3-C. In certain embodiments, each of the first and second Fe domains have the formula N-CH2-CH3-C.

g. 1+1+1 IL18-Fab (via VH)-scFv-Fc×empty-Fc

In some embodiments, the IL18 fusion is 1+1+1 IL18-Fab (via VH)-scFv-Fc×empty-Fe that includes (a) a first monomer comprising from N-terminus to C-terminus: an IL-18 protein, a first variable heavy domain (VH1), an scFv, and a first Fe domain; (b) a second monomer comprising a second Fe domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen. The Fc chain of the first monomer and the Fc chain of the second monomer form a heterodimeric Fc complex. See, the schematic diagram in FIG. 118G and the amino acid sequences of FIG. 123 of U.S. 63/502,344, hereby incorporated by reference.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 1+1+1 IL18-Fab (via VH)-scFv-Fc×empty-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111 Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110S/N111R/M113V, M51K/K53G/Q56G/P57A/M60L/Q103E/S105D/D110S/N111G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V11I/C38A/K53A/T63A/C76A/C127A.

In some embodiments, the IL18 variant of the 1+1+1 IL18-Fab (via VH)-scFv-Fc×empty-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the 1+1+1 IL18-Fab (via VH)-scFv-Fc×empty-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections VI-VIII above.

In exemplary embodiments, the 1+1+1 IL18-Fab (via VH)-scFv-Fc×empty-Fc fusion protein is a heterodimeric Fc fusion protein. In some heterodimeric embodiments, the first and second Fe domains include the amino acid substitution set L368D/K370S. S364K/E357Q. In some embodiments, the S364K/E357Q modifications are in the first Fe domain and the L368D/K370S modifications are in the second Fe domain. In some embodiments, the S364K/E357Q modifications are in the second Fe domain and the L368D/K370S modifications are in the first Fe domain. In certain heterodimeric embodiments, the first or second Fe domain includes isosteric pI variants Q295E/N384D/Q418E/N421D. In certain embodiments, both the first and second Fe domains include FcKO variants:E233P/L234V/L235A/G236del/S267K, according to the EU numbering. In some embodiments, the first Fe domain and the second Fe domain each include K447del modifications. In some embodiments, the IL18 protein or variant thereof is linked to the Fe domain that includes isosteric pI variants (e.g., the second Fe domain).

In exemplary embodiments, the 1+1+1 IL18-Fab (via VH)-scFv-Fc×empty-Fc fusion protein is a heterodimeric Fc fusion protein containing a first monomer, a second monomer and a third monomer. In some embodiments, the first monomer includes a variable heavy chain, the second monomer includes an IL18 protein or variant thereof, and the third monomer includes a variable light chain. In some embodiments, the first monomer includes a first Fe domain with heterodimer skew variants S364K/E357Q and FcKO variants E233P/L234V/L235A/G236del/S267K, according to the EU index. In some embodiments, the second monomer includes a second Fe domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fe domain with modifications C220S/E233P/L234V/L235A/G236del/S267K/S364K/E357Q and optionally modifications M428L/N434S, the second monomer includes a first Fe domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIG. 123 of U.S. 63/502,344, hereby incorporated by reference, depict amino acid modifications in the first and second monomers of a heterodimeric monovalent IL18-Fc fusion protein. Additional, exemplary Fc domain “backbone sequences” that find use in the subject 1+1+1 IL18-Fab (via VH)-scFv-Fc×empty-Fc are depicted in FIGS. 9A-9E and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

h. 1+1+1 IL18-Fab (via VL)-scFv-Fc×empty-Fc

In some embodiments, the IL18 fusion is 1+1+1 IL18-Fab (via VL)-scFv-Fc×empty-Fc that includes (a) a first monomer comprising from N-terminus to C-terminus: a first variable heavy domain (VH1), an scFv, and a first Fc domain; (b) a second monomer comprising a second Fc domain; and (c) a third monomer comprising from N-terminus to C-terminus: an IL-18 protein, a first variable light domain (VL1), and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen. The Fc chain of the first monomer and the Fc chain of the second monomer form a heterodimeric Fc complex. See, the schematic diagram in FIG. 118H and the amino acid sequences of FIG. 124 of U.S. 63/502,344, hereby incorporated by reference.

In some embodiments, the second antigen is human CD3ε. The CD3 ABDs of the invention bind to the extracellular domain of human CD3ε. In some embodiments, the second ABD comprises any of the variable heavy and light domain pairs as shown in FIG. 120 and described herein as well as any ABDs that compete for binding with the FIG. 120 ABDs and those described herein.

In some embodiments, the first antigen is B7H3 and the B7H3 ABD binds to the extracellular domain of human B7H3. In some embodiments, the first ABD comprises any of the variable heavy and light domain pairs as shown in FIGS. 146, 147, 148, and 149 and described herein. In some embodiments, the first ABD comprises any ABD that competes for binding with the FIGS. 146, 147, 148, and 149 ABDs and those described herein. In some embodiments, the first antigen is EGFR and the EGFR ABD binds to the extracellular domain of human EGFR. In some embodiments, the first ABD comprises any of the variable heavy and light domain pairs as shown in FIG. 150 and described herein. In some embodiments, the first ABD comprises any ABD that competes for binding with the FIG. 150 ABDs and those described herein. In some embodiments, the first antigen is HER2 and the HER2 ABD binds to the extracellular domain of human HER2. In some embodiments, the first ABD comprises any of the variable heavy and light domain pairs as shown in FIG. 151 and described herein. In some embodiments, the first ABD comprises any ABD that competes for binding with the FIG. 151 ABDs and those described herein. In some embodiments, the first antigen is CD19 and the CD19 ABD binds to the extracellular domain of human CD19. In some embodiments, the first ABD comprises any of the variable heavy and light domain pairs as shown in FIG. 152 and described herein. In some embodiments, the first ABD comprises any ABD that competes for binding with the FIG. 152 ABDs and those described herein. In some embodiments, the first antigen is CD20 and the CD20 ABD binds to the extracellular domain of human CD20. In some embodiments, the first ABD comprises any of the variable heavy and light domain pairs as shown in FIG. 153 and described herein. In some embodiments, the first ABD comprises any ABD that competes for binding with the FIG. 153 ABDs and those described herein. In some embodiments, the first antigen is CD123 and the CD123 ABD binds to the extracellular domain of human CD123. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is CAIX and the CAIX ABD binds to the extracellular domain of human CAIX. In some embodiments, the first ABD comprises any of the variable heavy and light domain pairs as shown in FIG. 154 and described herein. In some embodiments, the first ABD comprises any ABD that competes for binding with the FIG. 154 ABDs and those described herein. In some embodiments, the first antigen is FLT3 and the FLT3 ABD binds to the extracellular domain of human FLT3. In some embodiments, the first ABD comprises any of the variable heavy and light domain pairs as shown in FIG. 155 and described herein. In some embodiments, the first ABD comprises any ABD that competes for binding with the FIG. 155 ABDs and those described herein. In some embodiments, the first antigen is MSLN and the MSLN ABD binds to the extracellular domain of human MSLN. In some embodiments, the first ABD comprises any of the variable heavy and light domain pairs as shown in FIG. 156 and described herein. In some embodiments, the first ABD comprises any ABD that competes for binding with the FIG. 156 ABDs and those described herein. In some embodiments, the first antigen is Trop2 and the Trop2 ABD binds to the extracellular domain of human Trop2. In some embodiments, the first ABD comprises any of the variable heavy and light domain pairs as shown in FIG. 157 and described herein. In some embodiments, the first ABD comprises any ABD that competes for binding with the FIG. 157 ABDs and those described herein. In some embodiments, the first antigen is CEA and the CEA ABD binds to the extracellular domain of human CEA. In some embodiments, the first ABD comprises any of the variable heavy and light domain pairs as shown in FIG. 158 and described herein. In some embodiments, the first ABD comprises any ABD that competes for binding with the FIG. 158 ABDs and those described herein. In some embodiments, the first antigen is CLDN18.2 and the CLDN18.2 ABD binds to the extracellular domain of human CLDN18.2. In some embodiments, the first ABD comprises any of the variable heavy and light domain pairs as shown in FIG. 159 and described herein. In some embodiments, the first ABD comprises any ABD that competes for binding with the FIG. 159 ABDs and those described herein. In some embodiments, the first antigen is BCMA and the BCMA ABD binds to the extracellular domain of human BCMA. In some embodiments, the first ABD comprises any of the variable heavy and light domain pairs as shown in FIG. 160 and described herein. In some embodiments, the first ABD comprises any ABD that competes for binding with the FIG. 160 ABDs and those described herein. In some embodiments, the first antigen is PD-1 and the PD-1 ABD binds to the extracellular domain of human PD-1. In some embodiments, the first ABD comprises any of the variable heavy and light domain pairs as shown in FIG. 161 and described herein. In some embodiments, the first ABD comprises any ABD that competes for binding with the FIG. 161 ABDs and those described herein. In some embodiments, the first antigen is ANO1 and the ANO1 ABD binds to the extracellular domain of human ANO1. In some embodiments, the first ABD comprises any of the variable heavy and light domain pairs described herein. In some embodiments, the first ABD comprises any ABD that competes for binding with the ABDs described herein. In some embodiments, the first antigen is CD22 and the CD22 ABD binds to the extracellular domain of human CD22. In some embodiments, the first ABD comprises any of the variable heavy and light domain pairs described herein. In some embodiments, the first ABD comprises any ABD that competes for binding with the ABDs described herein. In some embodiments, the first antigen is CD38 and the CD38 ABD binds to the extracellular domain of human CD38. In some embodiments, the first ABD comprises any of the variable heavy and light domain pairs described herein. In some embodiments, the first ABD comprises any ABD that competes for binding with the ABDs described herein. In some embodiments, the first antigen is GPC3 and the GPC3 ABD binds to the extracellular domain of human GPC3. In some embodiments, the first ABD comprises any of the variable heavy and light domain pairs as shown in SEQ ID NOS:2422-2537. In some embodiments, the first ABD comprises any ABD that competes for binding with the ABDs of SEQ ID NOS:2422-2537 and those described herein.

Any of the IL18s described herein can be included in the 1+1+1 IL18-Fab (via VL)-scFv-Fc×empty-Fc. In some embodiments, the IL18 is wildtype mature human IL18 (FIG. 1A). In certain embodiments, the IL18 is a variant IL18 that includes one or more modifications as depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E and described above. In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y. In some embodiments, the amino acid substitution can include 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D1101R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, 4CS/E6Q/S10C/M51I/K53T/V153K/N155C M51T/M60K/S105D/D110K/N111H, M51T/S55K/G59A/M60K/S105D/D110K/N111H/V153I, Y1R/M51T/M60K/S105D/D110K/N111H, Y1R/M51T/K53R/M60K/S105N/D110K/N111Y, K8Q/M51T/S55K/G59T/M60K/S105R/D110H/N155K, K8R/M51K/S55K/G59A/M60Q/S105D/D110K/N111H/V153I, K8R/M51D/S55K/G59A/M60X/S105D/D110K/N111H/V153I, L5H/M51T/K53R/M60K/S105D/D110N/V153T, L5I/M51K/S55K/G59A/M60Q/S105K/D110Q/N111H/N155K, L5I/M51T/S55R/M60K/Q103E/S105D/D110H/N111H/V153I, L5I/M51T/S55K/M60K/S105D/D110K/N111H/V153T/N155H, L5I/M51T/S55K/G59A/M60K/S105R/D110H/N111H/V153I/N155K, L5I/K8R/M51T/S55K/M60K/S105D/N111Y/V153I/N155K, L5Y/K8R/M51T/K53R/M60K/S105D/D110E/N111H/N155K, Y1H/L5Y/M51T/K53R/M60K/S105D/D110H/N155K, Y1R/M51T/K53R/G59A/M60K/S105D/D110Q/N111H/V153A/N155K, Y1R/K8R/M51D/K53R/M60R/Q103K/S105N/D110K/N111Y/N155H, Y1R/K8R/M51N/K53R/M60Q/Q103K/S105R/D110N/N111H/N155K, Y1R/K8R/M51T/M60K/S105D/D110K/N111H, Y1R/L5H/M51T/K53R/M60K/Q103E/S105N/D110K/N111 Y, Y1R/K8R/M51T/K53R/G59A/M60K/Q103E/S105D/D110Q/N111H/V153I/N155X, Y1R/K8R/M51T/K53R/G59T/M60K/S105N/D110H/N111D/N155H, Y1R/K8R/M51T/G59A/M60K/Q103E/S105D/D110Q/N111H/V153I/N155K, Y1R/L5H/M51T/K53R/M60K/Q103E/S105N/D110K/N111 Y, Y1R/L5Y/M51T/G59T/M60K/E77D/S105D/D110K/N111H, Y1R/K8R/M51T/K53R/G59T/M60K/S105K/D110N/N111H/N155K, M51E/Q56E/P57L/M60R/Q103P/S105A/D110N/N111R/M113V, M51K/Q56A/P57G/M60L/Q103E/S105D/D110S/M113V, M51K/K53G/Q56A/P57A/M60L/D110K/N111R, M51K/K53G/Q56R/P57G/M60L/Q103E/S105D/D110N/N111 S/M113R, M51K/K53G/Q56V/M60L/Q103A/S105A/D110S/N111R/M113T, M51K/K53 S/Q56G/P57A/M60L/Q103A/S105A/D110G/N111R/M113T, M51K/K53S/Q56K/P57A/Q103A/S105D/D110S/N111S/M113R, M51K/K53 S/Q56L/P57A/M60L/S105D/D110S/N111R, M51K/K53 S/Q56R/P57A/M60L/S105N/D110G/N111R, M51K/K53 S/Q56R/P57A/M60L/Q103A/D110G/N111R/M113 T, M51K/K53 S/Q56R/P57A/M60L/Q103A/S105D/D110S/N111G/M113R, M51K/K53T/Q56R/M60L/Q103E/S105D/D110S/N111 S/M113K, M51K/K53T/Q56R/P57A/Q103E/S105D/D110N/N111D/M113R, M51R/Q56G/P57K/M60L/Q103R/D110 S/N111R/M113V, M51K/K53G/Q56G/P57A/M60L/Q103E/S105D/D110S/Ni11G/M113V, M51K/K53G/Q56R/S105A/D110N/N111R, M51K/K53 S/Q56L/P57A/M60L/S105D/D110S/N111R, M51K/K53 S/Q56R/P57A/M60L/Q103A/D110G/N111R/M113 T, M51K/K53 S/Q56R/P57A/M60L/S105N/D110G/N111R, M51K/K53G/Q56V/M60L/Q103A/S105A/D110S/N111R/M113T, M51K/K53 S/Q56R/P57A/M60L/Q103A/D110G/N111R/M113 T, M51K/K53S/Q56R/P57A/M60L/Q103A/S105D/D110S/N111G/M113R, K53A/P57T/M60A, G3Y/S10K/M51Q/K53A, C38S/K53A/P57T/M60A/C68S/C127S, G3Y/S10K/C38S/M51Q/K53A/C68S/C127S, M51A/K53G/Q56R/P57A/M60K, E6A/C38S/K53A/C68S/C76S/C127S, G3Y/E6A/C38S/K53A/C68S/C76S/C127S, G3L/E6A/C38S/K53A/C68S/C76S/C127S, E6W/C38S/K53A/C68S/C76S/C127S, E6A/T34P/C38S/K53A/C68S/C76S/C127S, E6A/C38M/K53A/C68S/C76S/C127S, E6A/C38S/M51Y/K53A/C68S/C76S/C127S, E6A/C38S/K53A/C68S/S72Y/C76S/C127S, E6A/C38S/K53A/C68S/S72F/C76S/C127S, E6A/C38S/K53A/C68S/S72M/C76S/C127S, E6A/C38S/K53A/C68S/S72L/C76S/C127S, E6A/C38S/K53A/C68S/S72W/C76S/C127S, E6A/C38S/K53A/C68S/C76S/K112W/C127S, E6A/C38S/K53A/C68S/C76S/S119V/C127S, E6A/C38S/K53A/C68S/C76S/C127S/G145N, E6A/S7C/C38S/S50C/K53A/C68S/C76S/C127S, G3Y/C38S/C68S/C76S/C127S, G3L/C38S/C68S/C76S/C127S, C38M/C68S/C76S/C127S, C38S/C68S/S72Y/C76S/C127S, C38S/C68S/S72F/C76S/C127S, C38S/C68S/S72M/C76S/C127S, E6K/V11I/C38A/K53A/T63A/C76A/C127A, V11I/C38A/M51G/K53A/C76A/C127A, E6K/V11I/C38A/M51G/K53A/T63A/C76A/C127A, N-terminal G/E6K/V11I/C38A/M51G/K53A/T63A/C76A/C127A, N-terminal G/E6K/V11I/C38A/K53A/T63A/C76A/C127A, or N-terminal 4G/E6K/V I/C38A/K53A/T63A/C76A/C127A.

In some embodiments, the IL18 variant of the 1+1+1 IL18-Fab (via VL)-scFv-Fc×empty-Fc includes one or more amino acid substitutions provided in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In some embodiments, the IL18 variant of the IL18-Fc fusion protein is depicted in any one of FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E. In certain embodiments, the IL18 variant includes an amino acid sequence set forth in SEQ ID NOS: 84-101, 196, 201, 277-284, 287-292, 296-298, 374-385, 697-702, 799-862, 864-949, 1265-1324, 1338, 1339, 1344-1361, and 1368.

Any Fc domains can be included in the 1+1+1 IL18-Fab (via VL)-scFv-Fc×empty-Fc fusion protein, including the wildtype and variant Fc domains described herein. In some embodiments, each Fc domain includes a CH2 and CH3. In certain embodiments, the first and second Fc domains include a hinge, CH2 and CH3. In one embodiment, the first and second Fc domains each have the formula, from N-terminus to C-terminus, hinge-CH2-CH3. In exemplary embodiments, the first and second Fc domains of the monovalent IL18-Fc fusion protein are heterodimeric. Modifications for such Fc domains are described in Sections above.

In exemplary embodiments, the 1+1+1 IL18-Fab (via VL)-scFv-Fc×empty-Fc fusion protein is a heterodimeric Fc fusion protein. In some heterodimeric embodiments, the first and second Fc domains include the amino acid substitution set L368D/K370S:S364K/E357Q. In some embodiments, the S364K/E357Q modifications are in the first Fc domain and the L368D/K370S modifications are in the second Fc domain. In some embodiments, the S364K/E357Q modifications are in the second Fc domain and the L368D/K370S modifications are in the first Fc domain. In certain heterodimeric embodiments, the first or second Fc domain includes isosteric pI variants Q295E/N384D/Q418E/N421D. In certain embodiments, both the first and second Fc domains include FcKO variants:E233P/L234V/L235A/G236del/S267K, according to the EU numbering. In some embodiments, the first Fc domain and the second Fc domain each include K447del modifications. In some embodiments, the IL18 protein or variant thereof is linked to the Fc domain that includes isosteric pI variants (e.g., the second Fc domain).

In exemplary embodiments, the 1+1+1 IL18-Fab (via VL)-scFv-Fc×empty-Fc fusion protein is a heterodimeric Fc fusion protein containing a first monomer, a second monomer and a third monomer. In some embodiments, the first monomer includes a variable heavy chain, the second monomer includes an IL18 protein or variant thereof, and the third monomer includes a variable light chain. In some embodiments, the first monomer includes a first Fc domain with heterodimer skew variants S364K/E357Q and FcKO variants E233P/L234V/L235A/G236del/S267K, according to the EU index. In some embodiments, the second monomer includes a second Fc domain with heterodimer skew variants L368D/K370S, isosteric pI variants Q295E/N384D/Q418E/N421D, and FcKO variants E233P/L234V/L235A/G236del/S267K.

In some embodiments, the first and second monomers each also include M428L/N434S half-life extension variants. In some embodiments, the first and second monomers each also include a C220S hinge amino acid substitution.

In some embodiments, the first monomer includes a second Fc domain with modifications C220S/E233P/L234V/L235A/G236del/S267K/S364K/E357Q and optionally modifications M428L/N434S, the second monomer includes a first Fc domain with modifications C220S/E233P/L234V/L235A/G236del/S267K/Q295E/L368D/K370S/384D/Q418E/N421D and optionally M428L/N434S, and according to the EU numbering and a third monomer that does not include an Fc domain. and according to the EU numbering. In some embodiments, the first Fc domain of the first monomer and the second Fc domain of the second monomer each include K447del modifications.

FIG. 124 of U.S. 63/502,344, hereby incorporated by reference, depict amino acid modifications in the first and second monomers of a heterodimeric IL18-Fc fusion protein. Additional, exemplary Fc domain “backbone sequences” that find use in the subject 1+1+1 IL18-Fab (via VL)-scFv-Fc×empty-Fc are depicted in FIGS. 9A-9E and 10.

In the formulas above, “IL18” is any IL18 provided herein (see, e.g., wildtype or variant IL18 depicted in FIGS. 13A-13B, 14, 15A-15D, 16A-16E, 17A-17B, 18, 19A-19P, 20A-20D, 31, 36, 37, 39A-39B, 40, 41A-41C, 42A-42D, 43A-43B, 44A-44C, 45, 46, 47, 48, 51, 54, 62, 87, and 88A-88E), “Fc domain” refers to any Fc domain provided herein (e.g., wildtype or variant Fc domains provided herein), and “linker” refers to any linker provided herein (see, e.g., FIG. 8). Further, “N” and “C” refer to the N-terminal and C-terminal orientation of each component in the second monomer. In such embodiments, the first monomer only includes an Fc domain (i.e., an “empty Fc domain”). In some embodiments, the each of the first and second Fc domains have the formula N-hinge-CH2-CH3-C. In certain embodiments, each of the first and second Fc domains have the formula N-CH2-CH3-C.

### XII. Nucleic Acids

In another aspect, provided herein are nucleic acid compositions encoding the subject IL18-Fc fusion proteins and IL18s (e.g., variant IL18s) described herein. As will be appreciated by those in the art, the nucleic acid compositions will depend on the format of the fusion protein. Thus, for example, when the format requires two amino acid sequences (e.g., heterodimeric IL18-Fc fusions), two nucleic acid sequences can be incorporated into one or more expression vectors for expression.

As is known in the art, the nucleic acids encoding the monomer components of the IL18-Fc fusion proteins can be incorporated into expression vectors as is known in the art, and depending on the host cells used to produce the heterodimeric IL18-Fc fusion proteins. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors are then transformed into any number of different types of host cells as is well known in the art, including, but not limited to, mammalian, bacterial, yeast, insect and/or fungal cells, with mammalian cells (e.g., CHO cells) being preferred.

In some embodiments, particularly heterodimeric IL18-Fc fusion proteins, nucleic acids encoding each monomer are each contained within a single expression vector, generally under different or the same promoter controls. In certain embodiments, each of the two nucleic acids are contained on a different expression vector.

The subject IL18-Fc fusion protein are made by culturing host cells comprising the expression vector(s) as is well known in the art. Once produced, traditional fusion protein or antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI variants that alter the isoelectric point (pI) of each monomer so that each monomer has a different pI, and the resulting heterodimeric IL18-Fc fusion protein also has a distinct pI advantageously facilitates isoelectric purification of the heterodimer (e.g., anionic exchange chromatography, cationic exchange chromatography). These substitutions also aid in the determination and monitoring of any contaminating homodimers post-purification (e.g., IEF gels, cIEF, and analytical IEX columns).

### XIII. Biological and Biochemical Functionality of IL18 Fc Fusion Proteins

Biological activity of the subject IL18-Fc fusion proteins (including IL18× Fab-Fc fusion) and variant IL18s can be assessed using any IL18 activity assay known in the art. In exemplary in vitro assays, the test IL18-Fc fusion proteins can be used to stimulate the human myelomonocytic cell line, KG-1, which produces IFNγ and then upregulates PD-L1 which can be measured. IL18BP inhibition of the IL18-Fc fusion proteins can also be determined in such assays.

The effects of subject IL18-Fc fusion protein and variant IL18s on the proliferation of various lymphocyte populations can be assessed using any method for lymphocyte proliferation, for example, but not limited to CFSE dilution method, Ki67 intracellular staining of immune effector cells, and ³H-thymidine incorporation method.

Biological activity of the subject IL18-Fc fusion proteins can also be tested in vivo in an animal model, such as a Graft-versus-Host Disease (GVHD) model conducted in immunodeficient mice with engraftment of foreign immune cells (e.g., human PBMCs).

Generally, the subject IL18-Fc fusion proteins are administered to patients in need thereof (e.g., a patient with a cancer) and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

For example, evaluation of changes in immune status (e.g., presence of ICOS+CD4+ T cells following ipilimumab treatment) along with traditional measurements such as tumor burden, size, invasiveness, LN involvement, metastasis, etc. can be done. Thus, any or all of the following can be evaluated: the inhibitory effects of IL18 Fc fusions on CD4+ T cell activation or proliferation, CD8⁺ T (CTL) cell activation or proliferation, CD8⁺ T cell-mediated cytotoxic activity and/or CTL mediated cell depletion, NK cell activity and NK mediated cell depletion, the potentiating effects of IL18 Fc fusions on Treg cell differentiation and proliferation and Treg- or myeloid derived suppressor cell (MDSC)-mediated immunosuppression or immune tolerance, and/or the effects of IL18 Fc fusions on proinflammatory cytokine production by immune cells, e.g., IL-2, IFN-γ or TNF-α production by T or other immune cells.

In some embodiments, assessment of treatment is done by evaluating immune cell proliferation, using for example, CFSE dilution method, Ki67 intracellular staining of immune effector cells, and ³H-thymidine incorporation method.

In some embodiments, assessment of treatment is done by evaluating the increase in gene expression or increased protein levels of activation-associated markers

In general, gene expression assays are done as is known in the art.

In general, protein expression measurements are also similarly done as is known in the art.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, ⁵¹Cr or ³⁵S release method, LDH activity, MTT and/or WST assays, Calcein-AM assay, Luminescent based assay, Annexin V staining, Zombie Aqua™ staining and others.

In some embodiments, assessment of treatment is done by assessing T cell activity measured by cytokine production, measured either intracellularly or in culture supernatant using cytokines including, but not limited to, IFNγ, TNFα, GM-CSF, IL2, IL6, IL4, IL5, IL10, IL13 using well known techniques.

Accordingly, assessment of treatment can be done using assays that evaluate one or more of the following: (i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

### XIV. Treatments

Once made, the subject IL18-Fc fusion proteins find use in a number of oncology applications, such as by promoting IL18-Fc related immune cell activation (e.g., T cells are no longer suppressed) and proliferation.

Accordingly, the subject IL18-Fc fusion proteins provided find use in the treatment of these cancers.

1. Fusion Protein Compositions for In Vivo Administration

Formulations of the IL18-Fc fusion proteins used in accordance with the present invention are prepared for storage by mixing a fusion protein having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions.

2. Administrative Modalities

The IL18-Fc fusion proteins provided herein administered to a subject, in accord with known methods, such as intravenous administration as a bolus or by continuous infusion over a period of time.

3. Treatment Modalities

In the methods described herein, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MM) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the disclosure includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for cancer therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject.

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

### XV. Exemplary IL18 Constructs

1. Variant Human IL18 Protein

In one aspect, provided is a composition comprising a variant human IL18 protein, wherein the variant IL18 protein comprises a modification at one or more amino acid positions selected from the group including: Y1, E6, S7, K8, S10, V11, N14, L15, D17, Q18, D23, R27, P28, L29, E31, M33, T34, D35, S36, D37, C38, R39, D40, N41, R44, I46, I49, 550, M51, K53, D54, S55, Q56, P57, M60, A61, V62, T63, S65, K67, C68, E69, 171, C76, E77, 180, 181, N87, P88, D90, K93, T95, K96, S97, Q103, H109, D110, N111, M113, 5119, A126, C127, D132, L136, L138, K139, E141, L144, D146, R147, 1149, M150, V153, N155, E156, and D157, as compared to wildtype human IL18. In some instances, the variant human IL18 protein comprises an additional 1, 2, 3, 4, 5, 6, 7, 8, 9, or 10 amino acid modifications.

In some instances, the variant human IL18 protein comprises one or more amino acid substitutions selected from the group including: Y1F, Y1H, E6A, E6Q, S7C, S7P, K8E, K8Q, K8Y, K8R, K8D, K8N, K8S, K8T, S10C, V11, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, I49D, I49E, I49N, I49Q, I49Y, I49F, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, M60R, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, Q103Y, Q103E, Q103K, Q103R, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, V153E, V153K, V153R, V153Y, V153Q, V153N, V153D, N155C, E156Q, D157A, D157S, D157N, and D157del, as compared to wildtype human IL18.

In some embodiments, the variant human IL18 protein comprises amino acid substitutions selected from the group including: 4CS/S10C/K53T/M60K/N155C, 4CS/S10C/K53T/V153K/N155C, 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, wherein 4CS comprises the amino acid substitutions C38S/C68S/C76S/C127S. In some embodiments, the variant human IL-18 protein comprises amino acid substitutions selected from the group including: 4CS/S10C/K53T/M60K/N155C or 4CS/S10C/K53T/V153K/N155C.

In some embodiments, the variant human IL18 protein exhibits reduced binding affinity to the IL18 receptor 1 (IL18R1), the IL18 receptor accessory protein (IL18RAP), the IL18R1:IL18RAP complex and/or the IL18 binding protein (IL18BP), compared to wildtype human IL18. In some embodiments, the variant human IL18 protein exhibits reduced heterogeneity compared to wildtype human IL18. In some embodiments, the variant human IL18 protein exhibits improved production yield compared to wildtype human IL18. In some embodiments, the variant human IL18 protein exhibits improved stability compared to wildtype human IL18. The composition according to any one of claims 1-9, wherein the variant human IL18 protein exhibits modulated potency, compared to wildtype human IL18. In some embodiments, the variant human IL18 protein exhibits reduced IL18BP sink compared to wildtype human IL18.

2. Monovalent Fc Fusion

In another aspect, provided herein is a monovalent Fc fusion protein comprising: (a) a first monomer comprising from N-terminus to C-terminus: a wildtype or variant IL-18 protein and a first Fc domain; and (b) a second monomer comprising a second Fc domain.

In some embodiments, the wildtype IL18 protein has an amino acid sequence selected from the group including: SEQ ID NO:1 (human precursor IL18) and SEQ ID NO:2 (human mature IL18). In some embodiments, the variant IL18 protein has at least 90% sequence identity to an amino acid sequence selected from the group including: SEQ ID NO:1 (human precursor IL18) and SEQ ID NO:2 (human mature IL18).

In some embodiments, the variant IL18 protein comprises a modification at one or more amino acid positions selected from the group including: Y1, E6, S7, K8, S10, V11, N14, L15, D17, Q18, D23, R27, P28, L29, E31, M33, T34, D35, S36, D37, C38, R39, D40, N41, R44, 146, 149, S50, M51, K53, D54, S55, Q56, P57, M60, A61, V62, T63, S65, K67, C68, E69, 171, C76, E77, 180, 181, N87, P88, D90, K93, T95, K96, S97, Q103, H109, D110, N111, M113, S119, A126, C127, D132, L136, L138, K139, E141, L144, D146, R147, 1149, M150, V153, N155, E156, and D157, as compared to wildtype human IL18.

In some embodiments, the variant IL18 protein comprises one or more amino acid substitutions selected from the group including: Y1F, Y1H, E6A, E6Q, S7C, S7P, K8E, K8Q, K8Y, K8R, K8D, K8N, K8S, K8T, S10C, V11, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, I49D, 149E, I49N, I49Q, I49Y, I49F, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, M60R, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, Q103Y, Q103E, Q103K, Q103R, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, 1149V, M150F, M150T, V153E, V153K, V153R, V153Y, V153Q, V153N, V153D, N155C, E156Q, D157A, D157S, D157N, and D157del, as compared to wildtype human IL18.

In some embodiments, the variant IL18 protein comprises 4CS/S10C/K53T/M60K/N155C, 4CS/S10C/K53T/V153K/N155C, 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, wherein 4CS comprises the amino acid substitutions C38S/C68S/C76S/C127S. In some embodiments, the variant human IL-18 protein comprises amino acid substitutions 4CS/S10C/K53T/M60K/N155C or 4CS/S10C/K53T/V153K/N155C.

In some embodiments, the first Fe domain further comprise a set of amino acid substitutions Q295E/N384D/Q418E/N421D, according to EU numbering. In some embodiments, the first and/or second Fc domains further comprise an amino acid modification of K447del, according to EU numbering. In some embodiments, the first and/or second Fc domains further comprise a set of amino acid modifications selected from the group including: C219S, C220S, S228P, G236R/L328R, E233P/L234V/L235A/G236del/S239K, E233P/L234V/L235A/G236del/S239K/A327G, E233P/L234V/L235A/G236del/S267K/A327G, E233P/L234V/L235A/G236del, E233P/L234V/L235A/G236del/S267K, and C220S/E233P/L234V/L235A/G236del/S267K, according to EU numbering. In some embodiments, the first and second Fc domains each further comprise amino acid modifications C220S/E233P/L234V/L235A/G236del/S267K, according to EU numbering. In some embodiments, the Fc domain and the second Fc domain have a set of amino acid substitutions selected from the group including: (i) S267K/L368D/K370S:S267K/S364K/E357Q; (ii) S364K/E357Q:L368D/K370S; (iii) L368D/K370S:S364K; (iv) L368E/K370S:S364K; (v) T411E/K360E/Q362E:D401K; (vi) L368D/K370S:S364K/E357Q, and (vii) K370S:S364K/E357Q, according to EU numbering. In some embodiments, the first and second Fc domains further comprise amino acid substitutions M428L/N434S, according to EU numbering. In some embodiments, the first Fc domain comprises amino acid substitutions C220S/PVA_/S267K/L368D/K370S/M428L/N434S, and the second Fc domain comprises amino acid substitutions PVA_/S267K/S364K/E357Q/M428L/N434S, according to EU numbering.

In some embodiments, the wildtype or variant IL-18 protein is covalently attached to the N-terminus of the first Fc domain. In some embodiments, the wildtype or variant IL-18 protein is covalently attached to a domain linker which is covalently attached to the N-terminus of the first Fc domain. In some embodiments, the domain linker is selected from any one of the domain linkers in FIG. 8.

In another aspect, provided herein is a monovalent Fc fusion protein comprising: (a) a first monomer comprising from N-terminus to C-terminus: a variant IL-18 protein and a first Fc domain, wherein the variant human IL-18 protein comprises amino acid substitutions 4CS/S10C/K53T/M60K/N155C or 4CS/S10C/K53T/V153K/N155C, and wherein the variant IL-18 protein is covalently attached to the N-terminus of the first Fc domain; and (b) a second monomer comprising a second Fc domain, wherein the first Fc domain comprises amino acid substitutions C220S/PVA_/S267K/L368D/K370S/M428L/N434S, and the second Fc domain comprises amino acid substitutions PVA_/S267K/S364K/E357Q/M428L/N434S, according to EU numbering.

3. Fab-Fc

In one aspect, provided herein is a Fab-Fc fusion protein comprising: (a) a first monomer comprising from N-terminus to C-terminus: a variable heavy (VH) chain and a first Fc domain; (b) a second monomer comprising from N-terminus to C-terminus: a wildtype or variant IL-18 protein and a second Fc domain; and (c) a third monomer comprising a variable light (VL) chain, wherein the VH and VL form an antigen binding fragment (Fab).

In some embodiments, the wildtype IL18 protein has an amino acid sequence selected from the group including: SEQ ID NO:1 (human precursor IL18) and SEQ ID NO:2 (human mature TL18). In some embodiments, the variant TL18 protein has at least 90% sequence identity to an amino acid sequence selected from the group including: SEQ ID NO:1 (human precursor IL18) and SEQ ID NO:2 (human mature IL18). In some embodiments, the variant IL18 protein comprises a modification at one or more amino acid positions selected from the group including: Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, 171, C76, E77, 180, 181, N87, P88, D90, K93, T95, K96, S97, Q103, H109, D110, N111, M113, S119, A126, C127, D132, L136, L138, K139, E141, L144, D146, R147, 1149, M150, V153, N155, E156, and D157, as compared to wildtype human IL18.

In some embodiments, the variant IL18 protein comprising one or more amino acid substitutions selected from the group including: Y1F, Y1H, E6A, E6Q, S7C, S7P, K8E, K8Q, K8Y, K8R, K8D, K8N, K8S, K8T, S10C, V11, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, I49D, 149E, I49N, I49Q, I49Y, I49F, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, M60R, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, Q103Y, Q103E, Q103K, Q103R, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, 1149V, M150F, M150T, V153E, V153K, V153R, V153Y, V153Q, V153N, V153D, N155C, E156Q, D157A, D157S, D157N, and D157del, as compared to wildtype human IL18.

In some embodiments, the variant IL18 protein comprises 4CS/S10C/K53T/M60K/N155C, 4CS/S10C/K53T/V153K/N155C, 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, wherein 4CS comprises the amino acid substitutions C38S/C68S/C76S/C127S. In some embodiments, the variant human IL-18 protein comprises amino acid substitutions 4CS/S10C/K53T/M60K/N155C, 4CS/S10C/K53T/V153K/N155C, or 4CS/E6Q/S10C/K53D/N111T/N155C.

In some embodiments, the second Fe domain further comprise a set of amino acid substitutions Q295E/N384D/Q418E/N421D, according to EU numbering. In some embodiments, the first and/or second Fc domains further comprise a modification of K447del, according to EU numbering.

In some embodiments, the first and/or second Fc domains further comprise a set of amino acid substitutions selected from the group including: C219S, C220S, S228P, G236R/L328R, E233P/L234V/L235A/G236del/S239K, E233P/L234V/L235A/G236del/S239K/A327G, E233P/L234V/L235A/G236del/S267K/A327G, E233P/L234V/L235A/G236del, E233P/L234V/L235A/G236del/S267K, and C220S/E233P/L234V/L235A/G236del/S267K, according to EU numbering. In some embodiments, the first and second Fc domains each further comprise modifications C220S/E233P/L234V/L235A/G236del/S267K, according to EU numbering. In some embodiments, the first Fc domain and the second Fc domain have a set of amino acid substitutions selected from the group including: (i) S267K/L368D/K370S:S267K/S364K/E357Q; (ii) S364K/E357Q:L368D/K370S; (iii) L368D/K370S:S364K; (iv) L368E/K370S:S364K; (v) T411E/K360E/Q362E:D401K; (vi) L368D/K370S:S364K/E357Q, and (vii) K370S:S364K/E357Q, according to EU numbering. In some embodiments, the first and second Fc domains further comprise amino acid substitutions M428L/N434S, according to EU numbering. In some embodiments, the first Fc domain comprises amino acid substitutions C220S/PVA_/S267K/L368D/K370S/M428L/N434S, and the second Fc domain comprises amino acid substitutions PVA_/S267K/S364K/E357Q/M428L/N434S, according to EU numbering. In some embodiments, the wildtype or variant IL-18 protein is covalently attached to the N-terminus of the first Fc domain. In some embodiments, the wildtype or variant IL-18 protein is covalently attached to a domain linker which is covalently attached to the N-terminus of the first Fc domain. In some embodiments, the domain linker is selected from any one of the domain linkers in FIG. 8.

In some embodiments, provided herein is a Fab-Fc fusion protein comprising: (a) a first monomer comprising from N-terminus to C-terminus: a variable heavy (VH) chain and a first Fc domain; (b) a second monomer comprising from N-terminus to C-terminus: a variant IL-18 protein and a second Fc domain, wherein the variant human IL-18 protein comprises amino acid substitutions 4CS/S10C/K53T/M60K/N155C or 4CS/S10C/K53T/V153K/N155C; and (c) a third monomer comprising a variable light (VL) chain, wherein the VH and VL form an antigen binding fragment (Fab), wherein the first Fe domain comprises amino acid substitutions C220S/PVA_/S267K/L368D/K370S/M428L/N434S, and the second Fc domain comprises amino acid substitutions PVA_/S267K/S364K/E357Q/M428L/N434S, according to EU numbering.

4. 1+1+1 Fab-scFv-Fc×IL18-Fc

In one aspect, provided herein is an IL18 fusion protein of the “1+1+1 Fab-scFv-Fc×IL18-Fc” format comprising: (a) a first A monomer comprising from N-terminus to C-terminus: a first variable heavy domain (VH1), an scFv, and a first Fc domain; (b) a second monomer comprising from N-terminus to C-terminus: an IL-18 protein and a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen.

5. 1+1+1 scFv-Fc×IL18-Fab-Fc

In one aspect, provided herein is an IL18 fusion protein of the “1+1+1 Fab-scFv-Fc×IL18-Fc” format comprising: (a) a first monomer comprising from N-terminus to C-terminus: an scFv and a first Fc domain; (b) a second monomer comprising from N-terminus to C-terminus: an IL18 protein, a first variable heavy domain (VH1), and a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen.

6. 1+1+1 IL18-scFv-Fc× Fab-Fc

In one aspect, provided herein is an IL18 fusion protein of the “1+1+1 IL18-scFv-Fc× Fab-Fc” format comprising: (a) a first monomer comprising from N-terminus to C-terminus: an IL18 protein, an scFv, and a first Fc domain; (b) a second monomer comprising from N-terminus to C-terminus: a first variable heavy domain (VH1) and a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen.

7. 1+1+1 Fab-Fc-scFv×IL18-Fc

In one aspect, provided herein is an IL18 fusion protein of the “1+1+1 Fab-Fc-scFv×IL18-Fc” format comprising: (a) a first monomer comprising from N-terminus to C-terminus: a first variable heavy domain (VH1), a first Fc domain, and an scFv; (b) a second monomer comprising from N-terminus to C-terminus: an IL-18 protein and a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen.

8. 2+1+1 Fab-scFv-Fc×IL18-Fab-Fc

In one aspect, provided herein is an IL18 fusion protein of the “2+1+1 Fab-scFv-Fc×IL18-Fab-Fc” format comprising: (a) a first monomer comprising from N-terminus to C-terminus: a first variable heavy domain (VH1), an scFv, and a first Fc domain; (b) a second monomer comprising from N-terminus to C-terminus: an IL-18 protein, a second variable heavy domain (VH2), and a second Fc domain; and (c) a third monomer comprising from N-terminus to C-terminus: a first variable light domain (VL1) and a constant light domain, wherein the VH1 and the VL1 form a first antigen binding domain (ABD) to a first antigen, and wherein the VH2 and the VL1 form a second ABD to the first antigen, and wherein the scFv comprises a third variable heavy domain (VH3), a scFv linker, and a second variable light domain (VL2), wherein the VH3 and the VL2 together form a third ABD to a second antigen.

9. 2+1+1 Fab-Fab-Fc×IL18-scFv-Fc

In one aspect, provided herein is an IL18 fusion protein of the “2+1+1 Fab-Fab-Fc×IL18-scFv-Fc” format comprising: (a) a first monomer comprising from N-terminus to C-terminus: an IL-18 protein, an scFv, and a first Fc domain; (b) a second monomer comprising from N-terminus to C-terminus: a first variable heavy domain (VH1), a second variable heavy domain (VH2), and a second Fe domain; and (c) a third monomer comprising from N-terminus to C-terminus: a first variable light domain (VL1) and a constant light domain, wherein the VH1 and the VL1 form a first antigen binding domain (ABD) to a first antigen, and wherein the VH2 and the VL1 form a second ABD to the first antigen, and wherein the scFv comprises a third variable heavy domain (VH3), a scFv linker, and a second variable light domain (VL2), wherein the VH3 and the VL2 together form a third ABD to a second antigen.

10. 1+1+1 IL18-Fab (via VH)-scFv-Fc×empty-Fc

In one aspect, provided herein is an IL18 fusion protein of the “1+1+1 IL18-Fab (via VH)-scFv-Fc×empty-Fc” format comprising: (a) a first monomer comprising from N-terminus to C-terminus: an IL-18 protein, a first variable heavy domain (VH1), an scFv, and a first Fc domain; (b) a second monomer comprising a second Fc domain; and (c) a third monomer comprising from N-terminus to C-terminus: a first variable light domain (VL1) and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen.

11. 1+1+1 IL18-Fab (via VL)-scFv-Fc×empty-Fc

In one aspect, provided herein is an IL18 fusion protein of the “1+1+1 IL18-Fab (via VL)-scFv-Fc×empty-Fc” format comprising: (a) a first monomer comprising from N-terminus to C-terminus: a first variable heavy domain (VH1), an scFv, and a first Fc domain; (b) a second monomer comprising a second Fc domain; and (c) a third monomer comprising from N-terminus to C-terminus: an IL-18 protein, a first variable light domain (VL1), and a constant light domain, wherein the VH1 and the VL1 together form a first antigen binding domain (ABD) to a first antigen and wherein the scFv comprises a second variable heavy domain (VH2), a scFv linker, and a second variable light domain (VL2), wherein the VH2 and the VL2 together form a second ABD to a second antigen.

In some embodiments of any of the IL18 fusion proteins described herein, the second antigen is CD3. In some embodiments of any of the IL18 fusion proteins described herein, the second ABD comprises the VH and VL of any of the binding domains depicted in the Figures and sequence listing.

In some embodiments of any of the IL18 fusion proteins described herein, the first antigen is B7H3. In some embodiments of any of the IL18 fusion proteins described herein, the first antigen is MSLN. In some embodiments of any of the IL18 fusion proteins described herein, the first antigen is GPC3. In some embodiments of any of the IL18 fusion proteins described herein, the first antigen is EGFR. In some embodiments of any of the IL18 fusion proteins described herein, the first antigen is Trop2. In some embodiments of any of the IL18 fusion proteins described herein, the first antigen is CD20. In some embodiments of any of the IL18 fusion proteins described herein, the first antigen is FLT3. In some embodiments of any of the IL18 fusion proteins described herein, the first antigen is CD19. In some embodiments of any of the IL18 fusion proteins described herein, the first antigen is CD123. In some embodiments of any of the IL18 fusion proteins described herein, the first antigen is CD22. In some embodiments of any of the IL18 fusion proteins described herein, the first antigen is CD38. In some embodiments of any of the IL18 fusion proteins described herein, the first antigen is CEA. In some embodiments of any of the IL18 fusion proteins described herein, the first antigen is MSLN. In some embodiments of any of the IL18 fusion proteins described herein, the first antigen is BCMA. In some embodiments of any of the IL18 fusion proteins described herein, the first antigen is CAIX. In some embodiments of any of the IL18 fusion proteins described herein, the first antigen is CLDN18.2. In some embodiments of any of the IL18 fusion proteins described herein, the first antigen is HER2. In some embodiments of any of the IL18 fusion proteins described herein, the first antigen is PD-1. In some embodiments of any of the IL18 fusion proteins described herein, the first antigen is ANO1. In some embodiments of any of the IL18 fusion proteins described herein, the first ABD comprises the VH and VL of any of the binding domains depicted in the Figures and sequence listing.

In some embodiments of any of the IL18 fusion proteins described herein, the IL18 protein is a wildtype IL18 protein having an amino acid sequence selected from the group including: SEQ ID NO:1 (human precursor IL18) and SEQ ID NO:2 (human mature IL18).

In some embodiments of any of the IL18 fusion proteins described herein, the IL18 protein is a variant IL18 protein having at least 90% sequence identity to an amino acid sequence selected from the group including: SEQ ID NO:1 (human precursor IL18) and SEQ ID NO:2 (human mature IL18).

In some embodiments of any of the IL18 fusion proteins described herein, the variant IL18 protein comprises a modification at one or more amino acid positions selected from the group including: Y1, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, M60, A61, V62, T63, S65, K67, C68, E69, 171, C76, E77, 180, 181, N87, P88, D90, K93, T95, K96, S97, Q103, H109, D110, N111, M113, S119, A126, C127, D132, L136, L138, K139, E141, L144, D146, R147, 1149, M150, V153, N155, E156, and D157, as compared to wildtype human IL18.

In some embodiments of any of the IL18 fusion proteins described herein, the variant IL18 protein comprises one or more amino acid substitutions selected from the group including: Y1F, Y1H, E6A, E6Q, S7C, S7P, K8E, K8Q, K8Y, K8R, K8D, K8N, K8S, K8T, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, I49D, 149E, I49N, I49Q, I49Y, I49F, S50C, S50Y, M51I, M51K, M51Q, M51R, M51L, M51H, M51F, M51Y, K53A, K53D, K53E, K53G, K53H, K53I, K53L, K53M, K53N, K53Q, K53R, K53S, K53T, K53V, K53Y, K53F, D54C, S55N, S55Q, S55D, S55E, S55T, Q56I, Q56L, P57A, P57E, P57T, P57V, P57Q, P57D, P57Y, P57N, M60I, M60L, M60K, M60Y, M60F, M60R, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, Q103Y, Q103E, Q103K, Q103R, H109W, H109Y, D110N, D110Q, D110R, N111D, N111Q, N111S, N111T, N111E, M113I, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, V153E, V153K, V153R, V153Y, V153Q, V153N, V153D, N155C, E156Q, D157A, D157S, D157N, and D157del, as compared to wildtype human IL18.

In some embodiments of any of the IL18 fusion proteins described herein, the variant IL18 protein comprises 4CS/S10C/K53T/M60K/N155C, 4CS/S10C/K53T/V153K/N155C, 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/S10C/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, 4CS/E6Q/S10C/K53D/N111T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C.

In some embodiments of any of the IL18 fusion proteins described herein, the variant IL18 protein comprises amino acid substitutions selected from the group including: 4CS/S10C/K53T/M60K/N155C, 4CS/S10C/K53T/V153K/N155C, and 4CS/E6Q/S10C/K53D/N111T/N155C.

In some embodiments of any of the IL18 fusion proteins described herein, the first or second Fe domain further comprise a set of amino acid substitutions Q295E/N384D/Q418E/N421D, according to EU numbering.

In some embodiments of any of the IL18 fusion proteins described herein, the first and/or second Fc domains further comprise a modification of K447del, according to EU numbering.

In some embodiments of any of the IL18 fusion proteins described herein, the first and/or second Fc domains further comprise a set of amino acid substitutions selected from the group including: C219S, C220S, S228P, G236R/L328R, E233P/L234V/L235A/G236del/S239K, E233P/L234V/L235A/G236del/S239K/A327G, E233P/L234V/L235A/G236del/S267K/A327G, E233P/L234V/L235A/G236del, E233P/L234V/L235A/G236del/S267K, and C220S/E233P/L234V/L235A/G236del/S267K, according to EU numbering.

In some embodiments of any of the IL18 fusion proteins described herein, the first and second Fc domains each further comprise modifications C220S/E233P/L234V/L235A/G236del/S267K, according to EU numbering.

In some embodiments of any of the IL18 fusion proteins described herein, the first Fc domain and the second Fc domain have a set of amino acid substitutions selected from the group including: (i) S267K/L368D/K370S:S267K/S364K/E357Q; (ii) S364K/E357Q:L368D/K370S; (iii) L368D/K370S:S364K; (iv) L368E/K370S:S364K; (v) T411E/K360E/Q362E:D401K; (vi) L368D/K370S:S364K/E357Q, and (vii) K370S:S364K/E357Q, according to EU numbering.

In some embodiments of any of the IL18 fusion proteins described herein, the first and second Fc domains further comprise amino acid substitutions M428L/N434S, according to EU numbering.

12. IL18×CD3×TTA Trispecific Fusion Protein Complex

In one aspect, provided herein is a trispecific fusion protein complex comprising: a) an IL-18 protein; b) an antigen binding domain (ABD) that binds to the extracellular domain of human CD3ε; and c) an ABD that binds to the extracellular domain of a human tumor target antigen (TTA).

In some embodiments, the IL-18 protein is a variant human IL-18 protein. In some embodiments, the variant human IL-18 has amino acid modifications selected from the group including: K53T/M60K, 4CS/S10C/K53T/M60K/N155C, 4CS/S10C/K53T/V153K/N155C, 4CS/E6Q/S10C/K53D/N111T/N155C, 4CS, 4CS/D193S, 4CS/D193A, 4CS/delD193, 4CS/S38E, 4CS/S68E, 4CS/S76E, 4CS/S127E, 4CS/S38K, 4CS/S68K, 4CS/S76K, 4CS/S127K, 4CS/S38D, 4CS/Y1F, 4CS/Y1H, 4CS/E6A, 4CS/E6Q, 4CS/D17N, 4CS/E31Q, 4CS/D35N, 4CS/D37N, 4CS/D40N, 4CS/N41Q, 4CS/K53R, 4CS/K53H, 4CS/K53M, 4CS/K53E, 4CS/K53Q, 4CS/K53A, 4CS/Q103E, 4CS/D110N, 4CS/N111Q, 4CS/E6A/K53A, 4CS/N14C/E31Q/S127C, 4CS/E31Q/K53A, 4CS/E31Q/D35N/K53A, 4CS/E31Q/N41Q/K53A, 4CS/E31Q/D35N/N41Q/K53A, 4CS/E31Q/D35N, 4CS/E31Q/N41Q, 4CS/E31Q/D35N/N41Q, 4CS/E31Q/D37N, 4CS/E31Q/D37N/K53A, 4CS/E31Q/M33C/S38C, 4CS/E31Q/S76C/L138C, 4CS/E31Q/S68I, 4CS/E31Q/S68F, 4CS/E31Q/S127W, 4CS/E31Q/S127Y, 4CS/E31Q/S127F, 4CS/S10C/E31Q/I49C, 4CS/L15C/E31Q/R147C, 4CS/P28C/E31Q/L136C, 4CS/E31Q/S50C/P88C, 4CS/E31Q/T63C/P88C, 4CS/E31Q/V62C/Q103C, 4CS/S10C/E31Q/N155C, 4CS/E31Q/S65C/P88C, 4CS/S7C/E31Q/S50C, 4CS/E31Q/D54C/A61C, 4CS/E31Q/A126C/K139C, 4CS/N14W/E31Q, 4CS/E31Q/D146Y, 4CS/E31Q/D146L, 4CS/E31Q/D146F, 4CS/E31Q/Q103L, 4CS/E31Q/Q103I, 4CS/E31Q/M150F, 4CS/Q18L/E31Q, 4CS/E31Q/S68Y, 4CS/E31Q/S38Q, 4CS/E31Q/S38R, 4CS/E31Q/S68D, 4CS/S7P/E31Q, 4CS/V11I/E31Q, 4CS/D23N/E31Q, 4CS/D23S/E31Q, 4CS/R27Q/E31Q, 4CS/L29V/E31Q, 4CS/E31Q/T34P, 4CS/E31Q/R39T, 4CS/E31Q/R39S, 4CS/E31Q/R44Q, 4CS/E31Q/I46V, 4CS/E31Q/S50Y, 4CS/E31Q/Q56L, 4CS/E31Q/Q56L/P57T, 4CS/E31Q/P57T, 4CS/E31Q/P57V, 4CS/E31Q/M60L, 4CS/E31Q/K67Q, 4CS/E31Q/E69K, 4CS/E31Q/I71M, 4CS/E31Q/E77K, 4CS/E31Q/I80T, 4CS/E31Q/I81V, 4CS/E31Q/I81L, 4CS/E31Q/N87S, 4CS/E31Q/D90E, 4CS/E31Q/K93D/T95E, 4CS/E31Q/K93N/T95E, 4CS/E31Q/T95E, 4CS/E31Q/K96G, 4CS/E31Q/S97N, 4CS/E31Q/N111D, 4CS/E31Q/M113I, 4CS/E31Q/S119L, 4CS/E31Q/L144N, 4CS/E31Q/R147K, 4CS/E31Q/I149V, 4CS/E31Q/M150T, 4CS/E31Q/E156Q/D157N, 4CS/K53S, 4CS/K53G, 4CS/K53T, 4CS/K53I, 4CS/K53L, 4CS/K53N, 4CS/K53D, 4CS/M51K, 4CS/M51Q, 4CS/M51I, 4CS/S55N, 4CS/S55Q, 4CS/Q56L, 4CS/Q56I, 4CS/P57A, 4CS/P57E, 4CS/M60L, 4CS/M60I, 4CS/K8Y, 4CS/K8Q, 4CS/K8E, 4CS/H109W, 4CS/H109Y, 4CS/E31Q/S38E, 4CS/E31Q/S38L, 4CS/E31Q/S38I, 4CS/E31Q/S38V, 4CS/E31Q/S68N, 4CS/E31Q/S68E, 4CS/E31Q/S68Q, 4CS/E31Q/S76C, 4CS/E31Q/S127D, 4CS/E31Q/S127E, 4CS/D23N/E31Q/R27Q, 4CS/E31Q/Q56L/T95E, 4CS/E31Q/K96Q/S119L, 4CS/E31Q/E141K/I149V, 4CS/E31Q/E141Q/I149V, 4CS/S7P/E31Q/S50Y, 4CS/E31Q/I80T/I81L/delD193, 4CS/E31Q/P57A/S119L/delD193, 4CS/E31Q/P57A/I80T/I81L/S119L/delD193, 4CS/E31Q/P57A/K93D/T95E/S119L/delD193, 4CS/E31Q/I80T/S119L/delD193, 4CS/E31Q/I80T/I81L/K93D/T95E/delD193, 4CS/E31Q/P57A/I80T/I81L/K93D/T95E/S119L/delD193, 4CS/S7C/E31Q/S50C/delD193, 4CS/S7C/E31Q/S50C/P57A/delD193, 4CS/S7C/E31Q/S50C/S119L/delD193, 4CS/S7C/E31Q/S50C/I80T/delD193, 4CS/S7C/E31Q/S50C/I80T/S119L/delD193, 4CS/S7C/E31Q/S50C/P57A/I80T/S119L/delD193, 4CS/S10C/E31Q/N155C/delD193, 4CS/S10C/E31Q/P57A/N155C/delD193, 4CS/S10C/E31Q/S119L/N155C/delD193, 4CS/S10C/E31Q/I80T/N155C/delD193, 4CS/S10C/E31Q/I80T/S119L/N155C/delD193, 4CS/S10C/E31Q/P57A/I80T/S119L/N155C/delD193, 4CS/S10C/E31Q/I49C/delD193, 4CS/L15C/E31Q/R147C/delD193, 4CS/E31Q/T63C/P88C/delD193, 4CS/N14C/E31Q/S127C/delD193, 4CS/E31Q/S38R/S127W/delD193, 4CS/S10C/D35E/N155C, 4CS/S10C/S36D/N155C, 4CS/S10C/S36N/N155C, 4CS/S10C/K53V/N155C, 4CS/S10C/K53Y/N155C, 4CS/S10C/K53F/N155C, 4CS/S10C/M51R/N155C, 4CS/S10C/M51L/N155C, 4CS/S10C/M51H/N155C, 4CS/S10C/M51F/N155C, 4CS/S10C/M51Y/N155C, 4CS/S10C/S55D/N155C, 4CS/S10C/S55E/N155C, 4CS/S10C/S55T/N155C, 4CS/S10C/P57Q/N155C, 4CS/S10C/P57D/N155C, 4CS/S10C/P57Y/N155C, 4CS/S10C/P57N/N155C, 4CS/S10C/M60Y/N155C, 4CS/S10C/M60F/N155C, 4CS/S10C/D110Q/N155C, 4CS/S10C/D110R/N155C, 4CS/S10C/N111D/N155C, 4CS/S10C/N111S/N155C, 4CS/S10C/N111T/N155C, 4CS/S10C/N111E/N155C, 4CS/S10C/D132Q/N155C, 4CS/S10C/D132E/N155C, 4CS/E6Q/S10C/K53D/N155C, 4CS/E6Q/S10C/M51K/K53D/N155C, 4CS/S10C/E31Q/D35N/N41Q/K53A/N155C, 4CS/S10C/E31Q/N41Q/K53A/N155C, 4CS/S10C/E31Q/K53A/N155C, 4CS/S10C/K53T/N155C, 4CS/S10C/P57A/N155C, 4CS/S10C/N155C, 4CS/S10C/S76G/N155C, 4CS/S10C/S76A/N155C, 4CS/S10C/M51K/K53D/N155C, 4CS/S10C/M51K/K53E/N155C, 4CS/E6Q/S10C/K53E/N155C, 4CS/E6Q/S10C/M51K/K53E/N155C, 4CS/E6Q/S10C/M51K/P57E/N155C, 4CS/S10C/M51K/P57E/N155C, 4CS/E6Q/S10C/P57E/N155C, 4CS/S10C/E31Q/K53T/N155C, 4CS/S10C/K53G/P57E/N155C, 4CS/S10C/K53T/P57E/N155C, 4CS/S10C/K53A/P57E/N155C, 4CS/S10C/P57E/N155C, 4CS/S10C/K53D/N155C, 4CS/S10C/E31Q/N41Q/N155C, 4CS/S10C/K53A/N155C, 4CS/S10C/K53G/N155C, 4CS/S10C/K53E/N155C, 4CS/S10C/K53S/N155C, 4CS/S10C/M51L/K53D/N155C, 4CS/S10C/K53D/D110R/N155C, 4CS/S10C/K53D/N111T/N155C, 4CS/S10C/K53D/S55T/N155C, 4CS/S10C/K53D/S55T/D110R/N155C, 4CS/S10C/M51L/K53D/S55T/D110R/N111T/N155C, 4CS/S10C/M51L/K53D/S55T/D110R/N155C, 4CS/S10C/K53D/S55T/D110R/N111T/N155C, 4CS/S10C/K53D/S55T/N111T/N155C, 4CS/S10C/E31Q/D35N/N155C, 4CS/S10C/N41Q/N155C, 4CS/S10C/D35N/N155C, 4CS/S10C/D37N/N155C, 4CS/S10C/E31Q/D37N/N155C, 4CS/SOC/D35N/D37N/N155C, 4CS/E6Q/S10C/M51L/K53D/S55T/D110R/N111T/N155C, 4CS/S10C/K53D/H109Y/N155C, 4CS/S10C/D37N/K53D/N155C, 4CS/S10C/D35N/K53D/N155C, 4CS/K8E/S10C/K53D/N155C, 4CS/S10C/E31Q/K53D/N155C, 4CS/S10C/N41Q/K53D/N155C, 4CS/S10C/K53D/P57V/N155C, 4CS/S10C/K53D/P57T/N155C, E6A/K53A, D35N/K53A, N41Q/K53A, D35N/N41Q/K53A, D35N/N41Q, D37N/K53A, E6Q/K53D, E6Q/M51K/K53D, M51K/K53D, M51K/K53E, E6Q/K53E, E6Q/M51K/K53E, E6Q/M51K/P57E, M51K/P57E, E6Q/P57E, K53G/P57E, K53T/P57E, K53A/P57E, M51L/K53D, K53D/D110R, K53D/N111T, K53D/S55T, K53D/S55T/D110R, M51L/K53D/S55T/D110R/N111T, M51L/K53D/S55T/D110R, K53D/S55T/D110R/N111T, K53D/S55T/N111T, D35N/D37N, E6Q/M51L/K53D/S55T/D110R/N111T, K53D/H109Y, D37N/K53D, D35N/K53D, K8E/K53D, N41Q/K53D, K53D/P57V, K53D/P57T, E6Q/K53D/N111T, Q56L/P57T, K93D/T95E, K93N/T95E, E156Q/D157N, D23N/R27Q, Q56L/T95E, K96Q/S119L, E141K/I149V, E141Q/I149V, S7P/S50Y, 80T/81L, P57A/S119L, P57A/I80T/I81L/S119L, P57A/K93D/T95E/S119L, I80T/S119L, I80T/I81L/K93D/T95E, P57A/I80T/I81L/K93D/T95E/S119L, P57A/I80T/S119L, N14C/S127C, M33C/S38C, S76C/L138C, S10C/I49C, L15C/R147C, P28C/L136C, S50C/P88C, T63C/P88C, V62C/Q103C, S10C/N155C, S65C/P88C, S7C/S50C, D54C/A61C, A126C/K139C, C38R/C127W, E31Q/K53A, E31Q/D35N/K53A, E31Q/N41Q/K53A, E31Q/D35N/N41Q/K53A, E31Q/D35N, E31Q/N41Q, E31Q/D35N/N41Q, E31Q/D37N, E31Q/D37N/K53A, S10C/E31Q/I49C, L15C/E31Q/R147C, P28C/E31Q/L136C, E31Q/S50C/P88C, E31Q/T63C/P88C, E31Q/V62C/Q103C, S10C/E31Q/N155C, E31Q/S65C/P88C, S7C/E31Q/S50C, E31Q/D54C/A61C, E31Q/A126C/K139C, N14W/E31Q, E31Q/D146Y, E31Q/D146L, E31Q/D146F, E31Q/Q103L, E31Q/Q103I, E31Q/M150F, Q18L/E31Q, S7P/E31Q, V11I/E31Q, D23N/E31Q, D23S/E31Q, R27Q/E31Q, L29V/E31Q, E31Q/T34P, E31Q/R39T, E31Q/R39S, E31Q/R44Q, E31Q/I46V, E31Q/S50Y, E31Q/Q56L, E31Q/Q56L/P57T, E31Q/P57T, E31Q/P57V, E31Q/M60L, E31Q/K67Q, E31Q/E69K, E31Q/I71M, E31Q/E77K, E31Q/I80T, E31Q/I81V, E31Q/I81L, E31Q/N87S, E31Q/D90E, E31Q/K93D/T95E, E31Q/K93N/T95E, E31Q/T95E, E31Q/K96G, E31Q/S97N, E31Q/N111D, E31Q/M113I, E31Q/S119L, E31Q/L144N, E31Q/R147K, E31Q/I149V, E31Q/M150T, E31Q/E156Q/D157N, D23N/E31Q/R27Q, E31Q/Q56L/T95E, E31Q/K96Q/S119L, E31Q/E141K/I149V, E31Q/E141Q/I149V, S7P/E31Q/S50Y, E31Q/I80T/I81L/delD193, E31Q/P57A/S119L/delD193, E31Q/P57A/I80T/I81L/S119L/delD193, E31Q/P57A/K93D/T95E/S119L/delD193, E31Q/I80T/S119L/delD193, E31Q/I80T/I81L/K93D/T95E/delD193, E31Q/P57A/I80T/I81L/K93D/T95E/S119L/delD193, S7C/E31Q/S50C/delD193, S7C/E31Q/S50C/P57A/delD193, S7C/E31Q/S50C/S119L/delD193, S7C/E31Q/S50C/I80T/delD193, S7C/E31Q/S50C/I80T/S119L/delD193, S7C/E31Q/S50C/P57A/I80T/S119L/delD193, S10C/E31Q/N155C/delD193, S10C/E31Q/P57A/N155C/delD193, S10C/E31Q/S119L/N155C/delD193, S10C/E31Q/I80T/N155C/delD193, S10C/E31Q/I80T/S119L/N155C/delD193, S10C/E31Q/P57A/I80T/S119L/N155C/delD193, S10C/E31Q/I49C/delD193, L15C/E31Q/R147C/delD193, E31Q/T63C/P88C/delD193, S10C/D35E/N155C, S10C/S36D/N155C, S10C/S36N/N155C, S10C/K53V/N155C, S10C/K53Y/N155C, S10C/K53F/N155C, S10C/M51R/N155C, S10C/M51L/N155C, S10C/M51H/N155C, S10C/M51F/N155C, S10C/M51Y/N155C, S10C/S55D/N155C, S10C/S55E/N155C, S10C/S55T/N155C, S10C/P57Q/N155C, S10C/P57D/N155C, S10C/P57Y/N155C, S10C/P57N/N155C, S10C/M60Y/N155C, S10C/M60F/N155C, S10C/D110Q/N155C, S10C/D110R/N155C, S10C/N111D/N155C, S10C/N111S/N155C, S10C/N111T/N155C, S10C/N111E/N155C, S10C/D132Q/N155C, S10C/D132E/N155C, E6Q/S10C/K53D/N155C, E6Q/S10C/M51K/K53D/N155C, S10C/E31Q/D35N/N41Q/K53A/N155C, S10C/E31Q/N41Q/K53A/N155C, S10C/E31Q/K53A/N155C, S10C/K53T/N155C, S10C/P57A/N155C, S10C/M51K/K53D/N155C, S10C/M51K/K53E/N155C, E6Q/S10C/K53E/N155C, E6Q/S10C/M51K/K53E/N155C, E6Q/S10C/M51K/P57E/N155C, S10C/M51K/P57E/N155C, E6Q/S10C/P57E/N155C, S10C/E31Q/K53T/N155C, S10C/K53G/P57E/N155C, S10C/K53T/P57E/N155C, S10C/K53A/P57E/N155C, S10C/P57E/N155C, S10C/K53D/N155C, S10C/E31Q/N41Q/N155C, S10C/K53A/N155C, S10C/K53G/N155C, S10C/K53E/N155C, S10C/K53S/N155C, S10C/M51L/K53D/N155C, S10C/K53D/D110R/N155C, S10C/K53D/N111T/N155C, S10C/K53D/S55T/N155C, S10C/K53D/S55T/D110R/N155C, S10C/M51L/K53D/S55T/D110R/N111T/N155C, S10C/M51L/K53D/S55T/D110R/N155C, S10C/K53D/S55T/D110R/N111T/N155C, S10C/K53D/S55T/N111T/N155C, S10C/E31Q/D35N/N155C, S10C/N41Q/N155C, S10C/D35N/N155C, S10C/D37N/N155C, S10C/E31Q/D37N/N155C, S10C/D35N/D37N/N155C, E6Q/S10C/M51L/K53D/S55T/D110R/N111T/N155C, S10C/K53D/H109Y/N155C, S10C/D37N/K53D/N155C, S10C/D35N/K53D/N155C, K8E/S10C/K53D/N155C, S10C/E31Q/K53D/N155C, S10C/N41Q/K53D/N155C, S10C/K53D/P57V/N155C, S10C/K53D/P57T/N155C, E6Q/S10C/K53D/N111T/N155C, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53 T/M60K/V153K/N155C, 4CS/E6Q/S10C/K53 T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, and 4CS/E6Q/S10C/K53D/N111T/N155C, as compared to wildtype human IL18. In other embodiments, the variant IL18 protein comprises amino acid substitutions selected from the group including: 4CS/S10C/K53T/M60K/N155C, 4CS/S10C/K53T/V153K/N155C, and 4CS/E6Q/S10C/K53D/N111T/N155C. In some embodiments, the variant human IL-18 has reduced binding to IL-18BP. In some embodiments, the TTA is selected from the group including: EGFR, Trop2, CD20, B7H3, FLT3, CD19, CD123, CD22, CD38, CEA, MSLN, BCMA, CAIX, CLDN18.2, HER2, PD-1, and ANO1.

Provided herein are one or more nucleic acids encoding any one of the variant human IL18 proteins, monovalent Fc fusion proteins, Fab-Fc fusion proteins, IL18 fusion proteins, and trispecific fusion proteins described herein. Also provided is an expression vector includes any of the one or more nucleic acids described herein. Provided herein is a host cell includes any of the one or more nucleic acids described herein or any of the expression vectors described herein. In yet another aspect, provided is a method of making any one of the variant human IL18 proteins, monovalent Fc fusion proteins, Fab-Fc fusion proteins, IL18 fusion proteins, and trispecific fusion proteins described herein. In some embodiments, the method includes culturing any of the host cells described herein and recovering the variant human IL18 protein, monovalent Fc fusion protein, Fab-Fc fusion protein, IL18 fusion protein, or trispecific fusion protein from the cell culture.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publ. App. No. 2015/0307629, US Publ. App. No. 2014/0288275, U.S. Pat. No. 9,605,084 and WO 2014/145806, all of which are expressly incorporated by reference in their entirety and particularly for the techniques outlined therein.

### Example 1: Engineering and Production of IL18 Fusion Proteins

As depicted in FIG. 71, IL18R1 expression is biased towards NKs and memory T cell and can be targeted by IL18. As described above, cytokines such as IL18 have short half-life, and high dose treatment is required to achieve a concentration of cytokines at the target (e.g., tumor site) sufficient to induce an immune response. However, based on observations with other cytokines, high dose treatment with IL18 could potentially result in systemic toxicities. In order to address this issue, IL18 molecules were engineered as Fc fusions in various formats (collectively referred to hereon as IL18 fusion proteins or just IL18 fusions) with the aim to enhance serum half-life through FcRn-mediated recycling.

### 1A: IL18 Fusion Protein Formats

### 1A(a): IL18-Fc Fusions

Various IL18-Fc fusion formats were conceived. One such exemplary format of this category is the “monovIL18-Fc” format (cartoon schematic depicted in FIG. 12A) which comprises a first monomer comprising an IL18 monomer covalently attached to the N-terminus of a first heterodimeric Fc chain (optionally via a domain linker) and a second monomer comprising a complementary second heterodimeric Fc chain that is “Fc-only” or “empty-Fc”. Illustrative proteins of the monovIL18-Fc format are depicted in FIGS. 21A-21I and the sequence listing.

1A(b): IL18× Fab-Fc Fusions

For the reasons described in Example 2C, another category of IL18 fusions include a Fab arm. One such exemplary format of this category is the “IL18× Fab-Fc” format (cartoon schematic depicted in FIG. 12B) which comprises a first monomer comprising an IL18 monomer covalently attached to the N-terminus of a first heterodimeric Fc chain (optionally via a domain linker), a second monomer comprising a variable heavy (VH) region covalently attached to the N-terminus of a complementary second heterodimeric Fc chain, and a third monomer that is a corresponding light chain that forms a Fab with the second monomer. Illustrative proteins of the IL18× Fab-Fc format are depicted in FIGS. 22A-22DO.

### 1B: Production and Purification of IL18 Fusion Proteins

To produce IL18 fusions in the monovIL18-Fc or the IL18× Fab-Fc formats, plasmids coding for IL18 (WT or variant) was constructed by standard gene synthesis, followed by subcloning into pTT5 expression vectors containing Fc fusion partners (e.g., domain linkers and heterodimeric Fc backbones as depicted in FIGS. 8 and 9A-9E). For IL18 fusions in the monovIL18-Fc format, an additional plasmid encoding a complementary heterodimeric empty-Fc was used. Additionally, in the case of IL18 fusions in the IL18× Fab-Fc formats, plasmid coding for the variable heavy and variable light regions were constructed by standard gene synthesis, followed by subcloning into pTT5 expression vectors containing either Fc fusion partners (e.g. CH1 regions, domain linkers, and heterodimeric Fc backbones as depicted in FIGS. 8, 9A-9E, and 10) or constant light domain (e.g. Constant Light Domain—Kappa as depicted in FIG. 11). Proteins were produced by transient transfection of HEK293E or CHO cells with appropriate set of plasmids as described above.

As will be described in Example 2D, IL18 fusions may be engineered with Rapid Purification (RP) variants for ease of purification. IL18 fusions without the RP variants were purified via a two-step purification process comprising protein A chromatography (purification part 1) followed by ion exchange chromatography (purification part 2). IL18 fusions with the RP variants were purified via one-step purification comprising just protein A chromatography.

Post-purification material (from separation peaks) may be further characterized by analytical size-exclusion chromatography with multi-angle light scattering (aSEC-MALS) and analytical anion-exchange chromatography (aAEX) for identity, purity and homogeneity as generally described below.

For aSEC-MALS, the analysis was performed on an Agilent 1200 high-performance liquid chromatography (HPLC) system. Samples were injected onto a Superdex™ 200 10/300 GL column (GE Healthcare Life Sciences) at 1.0 mL/min using 1×PBS, pH 7.4 as the mobile phase at 4° C. for 25 minutes with UV detection wavelength at 280 nM. MALS was performed on a miniDAWN® TREOS® with an Optilab® T-rEX Refractive Index Detector (Wyatt Technology, Santa Barbara, CA). Analysis was performed using Agilent OpenLab Chromatography Data System (CDS) ChemStation Edition AIC version C.01.07 and ASTRA version 6.1.7.15.

For aAEX, the analysis was performed on an Agilent 1200 high-performance liquid chromatography (HPLC) system. Samples were injected onto a Proteomix SAX-NP5 5 μM non-porous column (Sepax Technologies, Inc., Newark, DE) at 1.0 mL/min using 0-40% NaCl gradient in 20 mM MES, pH 8.5 buffer with UV detection wavelength at 280 nM. Analysis was performed using Agilent OpenLAB CDS ChemStation Edition AIC version C.01.07.

### Example 2: Engineering IL18 Variants (and Additional Approaches) to Improve Production

In this section, engineering approaches to improve IL18 fusion production are described. Collectively, these include engineering approaches to improve yield as well as to decrease molecular heterogeneity and the variants are collectively referred to herein as production variants.

### 2A: Engineering IL18 to Remove Free Cysteines

Initially, IL18 fusion XENP30792 in the monovIL18-Fc format was produced using WT human IL18 (sequence as depicted in FIG. 1 for WT human IL18 mature form sequence; and in FIG. 21A for XENP30792). XENP30792 was produced and purified via the two-step purification process as generally described in Example 1B. FIG. 23A depicts the chromatogram showing purification part 2 of XENP30792 as a broad heterogeneous peak. The full pre-purified load and material from the broad peak were further characterized by aSEC-MALS which show that the material was predominantly heavy molecular weight species (FIG. 23B).

Based on the crystal structure of human IL18 as reported by Tsutsumi et al. (Nature Communications, 15 Dec. 2014, 5, 5340) [PDB code 3WO2] and modeling in Molecular Operating Environment (MOE; Chemical Computing Group, Montreal, Quebec, Canada), it was found that the four cysteine residues were spatially distant and unlikely to form intramolecular disulfide bridges. Accordingly, it was hypothesized that the heavy molecular weight species were aggregate material resulting from intermolecular mispairing of free cysteines (as would be consistent with Yamamoto 2004). This was likely not a problem for human IL18 recombinantly produced in E. coli as expression takes place at lower temperature and in a reduced environment. Therefore, an IL18 variant 4CS was engineered with C38S, C68S, C76S and C127S (sequence as depicted in FIG. 13). IL18 fusion XENP31296 with IL18-4CS in the monovIL18-Fc format (sequences as depicted in FIGS. 21A-21P) was produced and purified as generally described in Example 1B. FIG. 24A depicts the chromatogram showing purification part 2 of XENP31296 as 3 distinct peaks (P1, B, and A). The 3 peaks were further characterized by aSEC-MALS, and chromatograms are depicted in FIG. 24B along with MW of component species. The profiles show that peak B comprises a single species of ˜72.3 kD, which is consistent with the calculated molecular weight of XENP31296 (based on amino acid sequence) of 70.4 kD. Hereon, IL18 variant including only the 4CS substitution is referred to as WT-4CS.

### 2B: Additional Engineering to Remove Liabilities

In view of the potential impact of post-translational modification related liabilities as observed in Example 2A, additional engineering approaches were utilized to remove other potential liabilities.

One such approach was removal of the C-terminal lysine on the Fc regions. Therefore, each of the backbones as depicted in FIGS. 9A-9E (and in fact each of the IL18 fusion sequences depicted herein) may include a K447 deletion (K447_or K447del) in one or both Fc regions.

Another such approach was removal of a “DG” aspartic acid isomerization motif Initial IL18 fusion constructs were produced with IL18 covalently attached to a G4S linker. The C-terminal D of IL18 and the glycine residue introduces such a DG motif A first approach explored was removal of the G4S linker so that the IL18 is covalently attached to the N-terminal of E of the hinge region. This approach significantly reduced molecular heterogeneity; however, it drastically reduced yield. Additional approaches explored to remove the DG motif included deletion or substitution of the C-terminal D of IL18 (e.g., XENP31812, XENP31813, XENP31814, XENP38952 and XENP38953) or utilizing alternative linkers that do not introduce the DG motif (e.g., AG4 or EA3K as in XENP38954, XENP38955, XENP38956, and XENP38957).

### 2C: Engineering IL18-Fc with a Silent Fab Arm

Another approach conceived for improving the production of IL18 fusions was to produce the molecules with a Fab arm occupying the empty-Fc side. To avoid non-specific activity of the IL18 fusions, the Fab arm used was a silent Fv based on SEQ ID NOs:23 and 24 as disclosed in the sequence listing of WO 2020/078905.

FIG. 25A depicts the chromatogram showing purification part 2 of XENP37827 as 2 distinct peaks (B and C) which is an improvement over the purification profile of XENP31296 as 3 distinct peaks. Peak B was from purification part 2 was further characterized by aSEC-MALS, and chromatograms are depicted in FIG. 25B along with MW of component species. The profiles show that peak B comprises a single species of ˜119.7 kD which is consistent with the calculated molecular weight of XENP37827 (based on amino acid sequence) of 116.2 kD.

Surprisingly, this format also enabled enhanced yield (data not shown). Although the E31Q variant was later identified as a variant which enhanced yield, this format was advantageous for further screening of variants so that substitutions which may be detrimental to yield but otherwise advantageous for other properties (e.g., improving therapeutic index) could be recovered.

Additionally, alternative silent Fvs (e.g., germline sequences) may also be employed. An illustrative IL18 fusions in the IL18× Fab-Fc format utilizing the DP47GS germline sequence is depicted in FIG. 22Bu as XENP40686.

### 2D: Engineering IL18 Fusions for Rapid Purification (RP)

To simplify purification to enable higher throughput screening of IL18 variants, IL18 fusions were engineered with Rapid Purification or RP variants. Specifically, the empty-Fc or Fab-Fc chain was engineered with the H435R/Y436F variants to ablate Protein A binding. Combined with DNA ratio optimization which reduced formation of IL18-Fc homodimers, this approach enabled a single-step Protein A purification which removed empty-Fc homodimers (in the case of monovIL18-Fc) or Fab-Fc homodimers (in the case of IL18× Fab-Fc). Although many of the IL18 fusion sequences described herein include the RP variants, the IL18 fusions may be engineered and produced without the RP variants.

### 2E: E31Q Variant Enhances Yield

In engineering IL18 affinity variants as described in Example 3, the E31Q substitution was unexpectedly found to improve production yield. In combination with DNA ratio optimization, the E31Q substitution removed the necessity for engineering IL18 fusions in the IL18× Fab-Fc format.

### Example 3: Engineering IL18 Variants to Improve Therapeutic Index

In vivo, IL18's proinflammatory effect is attenuated by IFNγ-dependent expression of IL18BP (sequence depicted in FIGS. 1-3, respectively for human, mouse, and cynomolgus monkey). IL18BP binds free IL18 and prevents IL18 binding to IL18R1. In order to overcome the IL18BP sink, it would be necessary to dose IL18 fusions at high concentrations; however, this may lead to systemic toxicity.

It was reasoned that decreasing the affinity of IL18 for IL18 receptors (and by extension, decreasing their potency) would allow for higher dosing to overcome the IL18BP sink and extend the half-life of the IL18 fusion proteins. Alternatively, or in addition to this approach, it was reasoned that decreasing the affinity of IL18 for IL18BP would overcome this sink. Accordingly, IL18 variants were engineered with the aim to achieve one or both of these targets, and these variants are collectively referred to herein as affinity variants. It should be noted that variants with improved binding to IL18BP and/or the IL18 receptors were also identified and useful in certain contexts, and these variants are also considered as affinity variants.

### 3A: Kinetic Binding Assay

Binding of variant IL18 to IL18 receptors or IL18BP were investigated using Octet, a BioLayer Interferometry (BLI)-based method. Experimental steps for Octet generally include the following: Immobilization (capture of ligand to a biosensor); Association (dipping of ligand-coated biosensors into wells containing serial dilutions of the analyte); and Dissociation (returning of biosensors to well containing buffer) in order to determine the affinity of the test articles. A reference well containing buffer alone was also included in the method for background correction during data processing. In particular, ligands used were IL18R1, IL18R1×IL18RAP heterodimer, or IL18BP, and the analytes were IL18 fusions. Illustrative sequences for these antigens are depicted in FIGS. 1-3 (respectively for human, mouse, and cynomolgus monkey).

### 3B: In Vitro Assay

To screen for IL18 variants engineered for reduced activation via IL18 receptors or reduced neutralization by IL18BP, in vitro assays were also used. For these assays, human myelomonocytic cell line, KG-1, which produces IFNγ and upregulates PD-L1 in response to human IL18 was utilized.

In one assay format, dose dependent activation by IL18 fusions was investigated by stimulating KG-1 cells with increasing concentrations of IL18 fusions alone (to investigate reduced activation potency) or in the presence of fixed amount of IL18BP (to investigate reduced neutralization by IL18BP). IL18BP levels in NSCLC patient serum has been reported to be in the range of 10-50 ng/ml (or 0.5-3 nM). Accordingly in this assay format, 100 ng/ml (5.6 nM) IL18BP was added to simulate higher end of in vivo IL18BP levels.

In another assay format, the dose dependent neutralization effect of IL18BP was investigated by stimulated KG-1 cells with fixed amount of IL18 fusions in the presence of increasing concentrations of IL18BP.

Illustrative examples of these two assay formats utilizing recombinantly produced IL18 (MBL, Cat: #B001-5) and IL18BP (Acro, Cat. ILP-H5222) are depicted in FIGS. 26A-26B.

### 3C: Affinity Variants (Library 1)

Several approaches were used in a first round of engineering. Crystal structure of the IL18 signaling ternary complex as reported by Tsutsumi et al., supra [PDB code 3WO4] and crystal structure of human TL18 in complex with Ectromelia virus IL18 binding protein as reported by Krumm et al. (Proc Natl Acad Sci USA, 30 Dec. 2008, 105(52):20711-20715) [PDB code 3F62] were used to identify residues on IL18 which interacted with any part of the IL18 receptors or IL18BP. In particular, MOE software's free energy calculations (AMBER force field parameters) were used to calculate energy between amino acid residues at the IL18:IL18R1 interface or the IL18:IL18BP interface. This round primarily focused on residues such as D, E, N, and Q at which isosteric substitutions could be introduced (reasoning that isosteric substitutions have less potential for immunogenicity) and identified E6, D17, E31, D35, D37, D40, N41, Q103, D110, and N111 as suitable residues for engineering. However, Y1 and K53 were also identified by the free energy calculations. In particular, K53 was calculated to be a residue very important for IL18:IL18BP binding. Based on this analysis, a number of IL18 variants were engineered (sequences for which are depicted in FIGS. 15A-15D) and formatted as IL18× Fab-Fc fusions (sequences for which are depicted in FIGS. 22A-22DO as noted above in Example 2A, these variants were engineered on the 4CS background).

The variant IL18× Fab-Fc fusions were investigated in vitro as generally described above in Example 3B. In a first experiment, KG-1 cells were stimulated with increasing concentrations of IL18 fusions for 48 hours after which IL18 activity was evaluated by staining for PD-L1 expression as an indication of KG-1 cell activation. In a second set of experiments, KG-1 cells were stimulated with increasing concentrations of IL18 fusions for 48 hours in the absence or presence of fixed concentration of IL18BP. The data as depicted in FIG. 27 show that a number of the variants including E31Q, D35N, D37N, N41Q, K53M, and K53A decreased potency in KG-1 activation relative to WT-4CS IL18. The data as depicted in FIGS. 28A-28S show that for most of the variants, incubation with IL18BP shifts the activation potency; however, XENP38865 having the K53A appear minimally impacted by IL18BP (FIG. 28P) indicating that the K53A substitution reduces binding affinity and sink by IL18BP. It should be noted that, while not shown, molar equivalent (relative to XENP37827) of recombinant human IL-18 induces PD-L1 expression with similar potency as XENP37827.

In another set of experiments, KG-1 cells were stimulated with fixed concentration of IL18 fusions (10 nM) or molar equivalent of recombinant IL18 (5.6 nM) for 48 hours in the presence of increasing concentrations of IL18BP. IL18 activity was evaluated by staining for PD-L1 expression as an indication of KG-1 cell activation, data for which are depicted in FIGS. 29-30. Notably as depicted in FIG. 29 and consistent with the above, the specific substitution at K53A IL18's ability to escape IL18BP neutralization. For example, K53R substitution has no impact relative to WT. At this fixed concentration of IL18 fusion proteins, the K53M, K53Q and K53E variants exhibit slightly reduced inhibition potency relative to WT-4CS (as depicted in FIG. 29), but also exhibits reduced activation efficacy. The K53A variant exhibits reduced inhibition potency only.

Interestingly as seen in FIGS. 28C and 28D, the E6A and E6Q variants were found to improve binding to IL18R1. While this is opposite to the engineering target, these substitutions could be useful for introducing into very weak IL18R1-binding variants which may have otherwise advantageous properties (e.g., reduced IL18BP binding) to restore IL18R1 binding. Additionally, as this residue is clearly important for binding between IL18 and IL18R1, other substitutions at this position may conversely reduce binding to IL18R1.

### 3D: Affinity Variants (Library 2)

Following the success from the first library of affinity variants, a further library was made to identify additional residues for introducing substitutions as well as to investigate combinations of substitutions. This library focused on positions along the IL18:IL18BP interface to identify variants with decreased binding to IL18BP. As this interface overlaps with the IL18:IL18R1 interface, mutations may have an effect on interactions with both IL18BP and IL18R1. Accordingly, this library further focused on variants that drastically reduced IL18BP binding while minimally impacted IL18R1 binding; improved binding to IL18R1 while minimally impacting binding to IL18BP; or decreased binding to both IL18R1 and IL18RAP. Additional residues identified include K8, M51, S55, Q56, P57, M60, and H109. Additional IL18 variants were engineered (sequences for which are depicted in FIGS. 16A-16C and 17) and formatted as IL18× Fab-Fc fusions (sequences for which are depicted in FIGS. 22A-22DT; as above, these variants were engineered on the 4CS background).

Kinetic binding experiments as generally described in Example 3A were performed to investigate the binding of the variant IL18× Fab-Fc fusions to IL18R1 antigen, ILBP antigen, and IL18R1×IL18RAP heterodimer antigen, data for which are summarized in FIG. 31. Notably, a K53D single substitution variant (XENP40253) was found to have a weaker binding response to IL18R1 and IL18R1×RAP but advantageously also no binding response to IL18BP. The K53N single substitution variant (XENP40252) also abrogated binding to IL18BP, but also resulted in much weaker IL18R1×RAP binding. Additional observations made were as follows: several other substitutions at K53 also enabled weakened binding for both IL18R1 and IL18BP, albeit not to the same level of weakened IL18BP binding as K53D and K53N; S55N and S55Q enabled weakened binding to IL18R1 and IL18BP, although at the expense of stability (as depicted in FIG. 36); and M41K enabled enhanced binding for IL18R1.

Next, in vitro assays as generally described in Example 3B were performed to investigate activation potency of the variant IL18× Fab-Fc fusions. KG-1 cells stimulated with indicated concentrations of the IL18× Fab-Fc fusions for 48 hours after which cells were stained with anti-PDL1 mAb to evaluate IL18 activity. Data are depicted in FIGS. 32-35.

Notably, K53T may be a promising variant as it demonstrated good induction activity with very quick off-rate (as determined by Octet binding). Additionally, K53E demonstrated good induction of activity at higher concentration. P57E demonstrated similar induction of activity in comparison to K53E, but as will be described in Example 4C, the P57E additionally improves stability. The K53D and K53N variants demonstrated activity, which is promising, and in addition, the K53N variant improves stability (as depicted in Example 4C) possibly by introducing an N-linked glycosylation.

### Example 4: Engineering IL18 Variants to Improve Stability

### 4A: Stability Assay

Stability of the IL18 fusions were assessed using Differential Scanning Fluorimetry (DSF). DSF experiments were performed using a Bio-Rad CFX Connect Real-Time PCR Detection System. Proteins were mixed with SYPRO Orange fluorescent dye and diluted to 0.2 mg/mL in PBS. The final concentration of SYPRO Orange was 10×. After an initial 10-minute incubation period at 25° C., proteins were heated from 25 to 95° C. using a heating rate of 1° C./min. A fluorescence measurement was taken every 30 sec. Melting temperatures (Tm) were calculated using the instrument software. IL18× Fab-Fc fusions had a first unfolding event (Tm1) of −40° C. Generally, the stability of IgG1 is limited by the Tm1 of the Fc region at 66° C. Therefore, a IgG1-based Fc fusion with a Tm1 of −40° C. is suboptimal. Accordingly, IL18 variants were engineered with the aim to improve stability, and these variants are referred to herein as stability variants.

### 4B: Stepwise Reversion of C-S Substitutions

An initial avenue explored for improving stability was stepwise reversion of the substituted cysteines (from Example 2A) in the event that they were important for stability. Sequences of such IL18 variants are depicted in FIGS. 13A-13B and IL18× Fab-Fc fusions based on these variants are depicted in FIGS. 22A-22DO. In data not shown, restoration of cysteine at position 76 (i.e., C38S/C68S/C127S/D157_) improved Tm by 1.5° C. relative to 4CS/D157 in the context of IL18-G4S-His molecules.

### 4C: Certain Affinity Variants Improves Stability

In engineering IL18 affinity variants as described in Example 3C, the E31Q substitution was unexpectedly found to improve stability. Stability of the IL18× Fab-Fc fusions comprising the variants in Example 3C were investigated as described in Example 4A. It was found that while most of the IL18× Fab-Fc fusions had a Tm1 ranging from 35.5-42.5° C., XENP38855 having the E31Q IL18 variant had a Tm1 of 45° C. (FIG. 36). Furthermore, the improved stability conferred by the E31Q substitution is retained when combined with other variants, such as the combination variants as described in Example 3D (data not shown). Additionally in engineering the additional single substitution affinity variants in Example 3D, it was surprisingly found that K53N, P57A, and P57E advantageously increased Tm1 respectively by 5, 9.5, and 12° C.; notably, several substitutions, including alternative substitutions as the aforementioned positions, decreased Tm1 by as much as 11.5° C. (FIG. 37).

### 4D: Stability Variants (Library 1)

Several approaches were used in a first round of engineering. As in Example 3, MOE software's free energy calculations were used. The engineering focused on introducing disulfide bridges using existing cysteine residues, introducing disulfide bridges at novel sites, and mutating existing cysteine residues or core positions to hydrophobic residues. Additionally based on a sequence alignment of IL18 from various species such as Equus caballus, Bos taurus, and others (see FIG. 38), mutations to commonly observed residues were made. Based on these approaches, a number of IL18 variants were engineered (sequences for which are depicted in FIGS. 19A-19I; It should be noted that each of these stability variants include the 4CS substitutions, unless reversion to cysteine is explicitly denoted i.e., S38C, S68C, S76C, and/or S127C) and formatted as IL18× Fab-Fc fusions (sequences for which are depicted in FIGS. 22A-22DO). It should be noted that in the context of the IL18× Fab-Fc fusions, the variants all include E31Q to improve production (and the 4CS substitutions, unless reversion to cysteine explicitly called out (i.e., S38C, S68C, S76C, and/or S127C)). The data as depicted in FIG. 39 show a number of the variants improved Tm1 by up to 20° C. (relative to the E31Q variant having Tm1 of 45° C.) such as the disulfide bridge introducing S10C/N155C variant. Melting curves for illustrative His-tagged IL18 variants are shown in FIG. 72. Notably, these stabilizing disulfide bridges were able to overcome the requirement for E31Q.

### 4E: Stability Variants (Library 2)

Following the success from the first library of stability variants, a further library was made. The emphasis of this second library was to further explore native unpaired cysteines and mutate them to residues other than serine. Additionally, potentially interacting variants from the sequence alignment were mutated. Based on these approaches, a number of additional IL18 stability variants were engineered (sequences for which are depicted in FIGS. 20A-20C; It should be noted that each of these stability variants include the 4CS substitutions, unless alternative substitution at residues 38, 68, 76, and/or 127 is explicitly denoted e.g., S38E) and formatted as IL18× Fab-Fc fusions (sequences for which are depicted in FIGS. 22A-22DO). Stability was assayed as described in Example 4A, data for which are depicted in FIG. 40. As above, in the context of the IL18× Fab-Fc fusions, the variants all include E31Q to improve production (and the 4CS substitutions, unless alternative substitution at residues 38, 68, 76, and/or 127 is explicitly denoted e.g., S38E). Notably, substitution of S38 for other residues such as L, I, or V improved stability. Restoration of cysteine at residue 76 improved stability which is consistent with findings as described in Example 4B. E141K/I149V and E141Q/I149V also enabled minor improvement in stability.

### Example 5: Further IL18 Variants

Further IL18 variants were engineered based on the foregoing efforts. For example, in Further Variants (Library 1), favorite stability variants identified in Example 4 were combined (sequences for which are depicted in FIG. 41).

In Further Variants (Library 2), favorite positions identified in Affinity Variants (Libraries 1 and 2) (see Example 3C and 3D) were revisited and alternative amino acids at these positions were explored. Additionally, further structural modeling identified S36 and D132 as additional potential positions for engineering affinity modulating variants. Sequences for Further Variants (Library 2) are depicted in FIG. 42.

As identified Stability Variants (Library 1) (see Example 4D), disulfide bridge introducing S10C/N155C variant improved Tm by 20° C. and is suitable for combining with other variants; thus, in Further Variants (Library 3), favorite affinity variants were combined with S10C/N155C (sequences for which are depicted in FIG. 43).

As noted in Example 3D, K53D abrogated IL18BP binding, but also reduced IL18R1 and IL18R1×RAP binding. Additionally, as described in Example 3D, certain variants found to improve IL18R1 binding could be used to restore IL18R1 binding (and, by extension, activity). Accordingly in Further Variants (Library 4), combination variants were explored combining affinity variants (those that modulate affinity for IL18 receptors and/or those that modulate affinity for IL18BP (e.g., K53D)) (sequences for which are depicted in FIG. 44). In particular, IL18R1 enhancing mutations combined with K53D included E6Q, S55T, and/or N111T. As depicted in FIG. 62, K53D as in XENP41762 resulted in >1000-fold reduction in potency relative to WT XENP41756. Incorporating E6Q and N11iT as in XENP42006 resulted in ˜300-fold reduction in potency relative to WT XENP41756.

The new variants were formatted as IL18× Fab-Fc or monovIL18-Fc fusions (sequences for which are depicted in FIGS. 21-22) and assayed as described in Example 3A and 3B, data for which are depicted in FIGS. 48-62 and 74. It should be noted that while 100 ng/mL IL18BP were previously utilized, 1 μg/mL IL18BP were utilized in some of these additional experiments. As will be described in Example 7A, it was found that IL18BP was upregulated by the IL18 fusion proteins of the invention, and therefore, IL18 variants that can tolerate higher concentrations of TL18BP would be preferred.

It should be noted that variants across the various libraries were often stepwise combined with previous favorites. For example, while Further Variants (Library 4) was exploring combinations of affinity variants, each of the variants depicted in FIG. 44 further includes 4CS and S10C/N155C. Accordingly, FIGS. 45-47 summarizes positions and substitutions that were explored for each purpose (e.g., Affinity Variants vs. Stability Variants).

### Example 6: IL18× Fab-Fc vs. monovIL18-Fc

As with the incorporation off E31Q and S10C/N155C, it is possible to express stable IL18 fusions as either IL18× Fab-Fc or monovIL18-Fc (monovalent IL18-Fc), the activity of equivalent variants in either format was further investigated. The data depicted in FIG. 63 show that the IL18× Fab-Fc fusions have lower Ymax (lower efficacy) than their monovIL18-Fc counterpart. This both suggests that it is difficult to compare variants between formats and that it is important that engineering efforts to “fix” monovIL18-Fc fusions (e.g., introducing novel disulfide bridges) were successful.

### Example 7: In Vivo Characterization of Potency Reduced IL18 Fusions

To determine if the in vitro potency reduction in the IL18 fusions translated to activity in vivo, a graft-versus-host disease (GvHD) model conducted in NSG (NOD-SCID-gamma) immunodeficient mice was used. When the NSG mice are injected with human PBMCs, the human PBMCs develop an autoimmune response against mouse cells. Treatment of huPBMC-engrafted mice with IL18 fusions should activate and expand the engrafted human T cells and exacerbate disease.

### 7A: GvHD Study #1

In a first study, the aim was to examine biological activities of potency reduced TL18 fusions and explore how potency can be reduced while still maintaining activity. 10×10⁶ were engrafted into NSG mice on Day 0 along with 5 mg/kg or 0.5 mg/kg XENP40967 (3-fold reduction from WT), XENP40685 (17-fold reduction from WT), XENP40966 (219-fold reduction from WT), XENP40962 (210-fold reduction from WT), and XENP40965 (9220-fold reduction from WT). Mice were further dosed on Days 7, 14, and 21. Mice were weighed twice a week (change in body weight as an indicator of GvHD), data for which are depicted in FIG. 64. Blood was drawn on Day 7, 14, and 21 to investigate cytokine secretion and lymphocyte expansion and activation, data for which are shown in FIGS. 65-69. The data show that change in body weight, human T cell expansion, NK cell expansion, and cytokine secretion generally correlated with IL18 potency and was dose dependent. Additionally, the body weights and T cell counts correlated with the variants that were engineered to avoid IL18BP inhibition. The effect is less pronounced at higher 5 mg/kg doses, but at lower 0.5 mg/kg doses, the K53T and K53D variants as in XENP40967 and XENP40962 noticeably enhanced GVHD. While K53T likely enhances GVHD due to its higher potency, K53D is lower potency but enhances GVHD in comparison to variants with similar potency but which do not avoid IL18BP inhibition. Notably as depicted in FIG. 65, the IL18 fusions induced IL18BP. This further highlights the importance of detuning IL18BP binding.

### 7B: PK Study #1

To characterize the in vivo stability of the IL18 fusion proteins, PK experiments were performed in C57/B16 mice. Mice (n=4) were intravenously dosed on Day 0 with 2 mg/kg XENP39804 (monovIL18-Fc with 4CS/E31Q variant), XENP40685 (monovIL18-Fc with 4CS/E31Q variant and further including S10C/N155C), and XENP40686 (IL18× Fab-Fc with 4CS/E31Q variant and further including S10C/N155C). Blood was drawn 1 hour post dose and on Days 2, 5, 8, 13, and 16, and serum concentration of the IL18 fusion proteins was determined by anti-human IL18 detection antibody. PK interpretative analysis was performed using Phoenix WinNonlin software (Version 6.4.0.768) with PK parameters for non-compartmental analysis of free drug serum concentration versus time, data for which are shown in FIG. 70. The data show dramatic improvement in serum levels upon introduction of the S10C/N155C disulfide variant into the E31Q base and enabled antibody-like PK with an estimated half-life of 3 weeks. In addition, the IL18× Fab-Fc format did not appear to confer any further improvements.

### 7C: GvHD Study #2

In another study, dose dependent in vivo activity of XENP41770, XENP42006, and XENP41762 was investigated. 10×10⁶ were engrafted into NSG mice on Day 0 along with indicated concentrations of XENP41770, XENP42006, and XENP41762. Mice were further dosed on Days 7, 14, 21, and 28. Mice were weighed twice a week (change in body weight as an indicator of GvHD), data for which are depicted in FIG. 75. Notably by Day 8, 3 mg/kg XENP42006 enabled earliest enhanced GvHD (i.e., significantly enhanced in comparison to PBS control). Over the course of the study, XENP42006 continued to outperform XENP41770 and XENP41762 even at lower doses. Blood was drawn on to investigate cytokine secretion and lymphocyte expansion, data for which are shown in FIGS. 76-77.

### 7C: PK Study #2

Next, PK experiments were performed in cynomolgus monkeys. Monkeys were dosed on Day 0 and Day 14 with either XENP41974 (stabilized, WT affinity) and XENP42007 (stabilized, affinity-optimized which is XENP42006+M428L/N434S half-life extension Fc variant+Rapid Purification variant; note that XENP42143 is 42007 without the Rapid Purification variant). Data depicting total IL18-Fc vs. active IL18-Fc concentration over time are depicted in FIGS. 78-79. Total IL18-Fc includes IL18-Fc with and without bound IL18BP, while active IL18-Fc is unbound. The data show that active XENP41974 is rapidly cleared, while XENP42007 exhibits greatly improved PK (both slow receptor-mediated clearance and avoiding IL18BP sink). Additionally, the IL18-Fc was well tolerated and the cynomolgus monkeys exhibited no clinical observations.

### Example 8: Further Characterization of Fc Variants

A majority of the data depicted herein were in the context of IL18× Fab-Fc fusions or in the context of Fc fusions having Rapid Purification variants. Accordingly, it is important to confirm that the IL18 variants demonstrated their expected effect in the context of other Fc fusions having alternative variants (e.g., having M428L/N434S half-life extension Fc variant and excluding the Rapid Purification variant). Accordingly, the in vitro activity of XENP42141, XENP42143, and XENP42145 were investigated in a cytokine release assay. Human PBMC was stimulated with the IL18-Fc fusions plus human IL-12 and secretion of cytokine IP-10 was assessed. As expected, the data as depicted in FIG. 81 show that XENP42141 having WT potency most potently induced IP10 secretion while XENP42145 least potently induced IP10 secretion with XENP42143 (having the potency restoring E6Q/N111T variants) falling in between.

### Example 9: Identifying Additional IL18 Variants by Investigating Murine IL18

As depicted in Example 4D, engineering IL18 variants based on sequence alignment of IL18 from other species may be useful. Accordingly, additional murine IL18 variants were engineered (sequences as depicted in FIG. 80A-80R in the context of murine IL18-Fc fusions).

To screen these variants, mouse splenocytes were stimulated with murine TL18-Fc fusions plus murine IL-12 and activation of NK1.1 cells (indicated by % intracellular IFNγ+) was investigated, data for which are depicted in FIG. 82. Additionally, binding to murine IL18BP was also investigated as generally described herein, data for which are depicted in FIG. 83. Collectively, the following observations were made: L59K dramatically lowers affinity to IL18BP with beneficial effect on receptor potency; L59K in combination with K52X knocks out IL18BP binding; E55R and M50G are silent, even when combined with each other or with L59K; K52 variants modulates receptor affinity like corresponding K53 in human IL18 with K52A>K52V>K52D; E30 and D34 variants also modulate receptor affinity like corresponding E31 and D35 variants in human IL18 (although does not modulate IL18BP binding). As K52 variants in murine IL18 demonstrated similar effect as corresponding K53 variants in human IL18 (*note: corresponding based on alignment in FIG. 38), it is expected that other murine variants (e.g., at L59, and in particular, L59K) convey similar effect in human IL18. Accordingly, for example, it is expected that M60K in human IL18 (which corresponds to murine L59K based on alignment in FIG. 38) may reduce or knockout IL18BP binding and may be combined with K53 variants to further abrogate IL18BP binding. Similarly, it is expected that K53V in human IL18 may be useful in modulating receptor affinity.

### Example 10: IL18 Fusion Proteins Demonstrate Robust Activity In Vivo

CD34+Hu-NSG, which are NSG mice engrafted with human CD34+ hematopoietic stem cells so as to develop a functional human immune system with no reactivity towards the host, were obtained from The Jackson Laboratory (Bar Harbor, Maine). On Day −21, mice were intradermally inoculated with 3×10⁶ pp65-MCF7gfp tumor cells. Mice were then treated intraperitoneally on Days 0, 8, 14, 21, and 28 with XENP42006 (at 1 mg/kg or 3 mg/kg) alone or in combination with anti-PD1 mAb (3 mg/kg), anti-PD1 mAb alone, or PBS control. Tumor volumes were monitored by caliper measurements, data for which are shown in FIG. 86A for change in tumor volume by Day 14. Blood was drawn weekly to investigate lymphocyte expansion and granzyme B and IFNγ induction, data for which are depicted in FIGS. 86B-86F for Day 14. The data show MCF7 tumor growth is inhibited by IL18-Fc and anti-PD1, correlating with T cell and NK cell expansion, granzyme B and IFNγ induction, and Treg reduction.

### Example 11: Further IL18 Variants to Tune IL18BP Resistance and Potency

As noted above, although K53D essentially abrogated IL18BP binding, it also drastically reduced IL18R1 and IL18R1×RAP binding and was combined with additional substitutions such as E6Q, S55T, and N11iT to restore potency. An additional angle explored was to instead use K53T as the starting point, which although less effective in knocking out IL18BP binding was also less reduced in IL18R1 and IL18R1×RAP binding, and combining with further substitutions to abrogate IL18BP binding and/or restore IL18R1 and IL18R1×RAP binding. Such new positions and substitutions (both at previous and newly identified positions) explored are summarized in FIG. 87, and sequences for IL18 variants which include K53T and such additional substitutions (and combinations thereof) are depicted in FIG. 88. The new variants were formatted as IL18× Fab-Fc (sequences for which are depicted in FIGS. 22A-22DO) and assayed as described in Example 3A and 3B. It should be noted that while 100 ng/mL and 1 μg/ml IL18BP were previously utilized, 1 μg/mL IL18BP were utilized in these additional experiments.

In a first round of engineering (corresponding data for which are depicted in FIGS. 89-95), preferred variants included hIL18 with 4CS+S10C/N155C and either K53T/V153K (as in XENP44167) and K53T/M60K (as in XENP44161), which as depicted in FIG. 89 are respectively 9X and 88X reduced in activity compared to “WT” (which includes only 4CS+S10C/N155C), in comparison to XENP41431 which is 406X reduced in activity. While the K53T/V153K variants is more potent than the K53T/M60K variant, the latter (as depicted in FIG. 95) is more resistant to IL18BP (individually, V153K improves potency, while subtly improving IL18BP resistance; M60K improves IL18BP resistance but reduces potency).

Further variants were engineered by building on these preferred variants, corresponding data for which are depicted in FIGS. 96-106.

### Example 12: Investigating Further IL18 Variants in Mouse Tumor Model (Murine IL18 Surrogate)

Additional murine TL18 variants (sequences as depicted in FIG. 80 in the context of murine IL18-Fc fusions) were engineered with potency corresponding to further IL18 variants described in Example 11. To screen these variants, mouse splenocytes were stimulated with murine IL18-Fc fusions (in the context of monovIL18-Fc) and activation of NK1.1 cells (indicated by % intracellular IFNγ+) was investigated, data for which are depicted in FIG. 108. The data show that mIL18 variants having 3CS_A10C/M50G/K52V/E55R/L59K/N153C, 3CS_A10C/M50G/K52D/E55R/L59K/N153C, and 3CS_A10C/D34N/M50G/K52V/E55R/L59K/N153C (and AlOC/E30Q/M50G/K52A/E55R/L59K/N153C) substitutions respectively demonstrated 9-fold, 84-fold, and 336-fold reduction in potency from mIL18 “WT” having only 3CS_A10C/N153C stabilization variant corresponding respectively to potencies of hIL18 variants having 4CS_S10C/K53T/N153K/N155C, 4CS_S10C/K53T/M60K/N155C, and 4CS_S10C/E6Q/K53D/N155C (respectively having 9-fold, 88-fold, and 406-fold reduction in potency from hIL18 “WT” having only 4CS_S10C/N155C stabilization variants). These surrogate variants were also knocked down in mIL18BP binding as depicted in FIG. 117.

Next, in vivo activity of these murine IL-18 potency surrogates were investigated in a CT26 syngeneic model. BALB/c mice were inoculated subcutaneously in the right lower flank region with 5×10⁵ tumor CT26 colorectal tumor cells. Mice were randomized when mean tumor size reaches approximately 100 mm³, at which point treatment was initiated (Day 1). Mice were dosed weekly for 4 weeks with varying concentrations of XENP42594 (“WT” mIL18), XENP44058 (a comparator DR-mIL18, sequences depicted in FIG. 107), XENP43773 (mIL18 with 3CS_A10C/M50G/K52V/E55R/L59K/N153C having 9-foled reduced potency in comparison to “WT” mIL18), XENP43774 (mIL18 with 3CS_A10C/M50G/K52D/E55R/L59K/N153C having 84-fold reduced potency in comparison to “WT” mIL18), or XENP43772 (mIL18 with 3CS/A10D/D34N/M50G/K52V/E55R/L59K/N153C having 336-fold reduced potency in comparison to “WT” mIL18). Tumor volumes were measured with caliper twice per week after randomization, data for which are depicted in FIGS. 109 and 113. Total IL18 in serum from Day 9 was also assessed, data for which are depicted in FIG. 114. The data show that XENP43773 may be too potent leading to increased clearance while XENP43772 may be too weak; however, XENP43774 appears to hit a sweet spot of tradeoff between potency and PK. Notably, 0.3 mg/kg XENP43773 and XENP43774 demonstrated superior anti-tumor activity to equivalent dose of WT mIL18-mFc.

Additionally, RNA was extracted from the tumor infiltrates from Day 9 using RNeasy Plus Mini Kit (Qiagen, Hilden, Germany) and assayed by nCounter® Mouse Immunology Panel (NanoString Technologies, Seattle, Wash.). Fold change in gene expression following treatment with “WT” mIL18 vs. potency-reduced mIL18 were plotted in FIG. 115. The data show that in each case, gene expression profile correlated well suggesting that there should be no change in immune mechanism after engineering reduced potency.

Finally, 10 naïve Balb/c mice and 15 surviving mice from the above-described tumor study were engrafted with CT26 colorectal tumor cells. Tumor volumes were measured with caliper twice per week after randomization, data for which are depicted in FIG. 116, showing that tumor-free mice from engineered mIL18-Fc fusions prevent tumor growth upon rechallenge.

### Example 13: Further IL18 Variants to Tune IL18BP Resistance and Potency

As noted above, although K53D essentially abrogated IL18BP binding, it also drastically reduced IL18R1 and IL18R1×RAP binding and was combined with additional substitutions such as E6Q, S55T, and N111T to restore potency. An additional angle explored was to instead use K53T as the starting point, which although less effective in knocking out IL18BP binding was also less reduced in IL18R1 and IL18R1×RAP binding, and combining with further substitutions to abrogate IL18BP binding and/or restore IL18R1 and IL18R1×RAP binding. Such new positions and substitutions (both at previous and newly identified positions) explored are summarized in FIG. 87, and sequences for IL18 variants which include K53T and such additional substitutions (and combinations thereof) are depicted in FIG. 88. The new variants were formatted as IL18× Fab-Fc (sequences for which are depicted in FIGS. 22A-22DO) and assayed as described in Example 3A and 3B. It should be noted that while 100 ng/mL and 1 μg/ml IL18BP were previously utilized, 1 μg/mL IL18BP were utilized in these additional experiments.

In a first round of engineering (corresponding data for which are depicted in FIGS. 89-95), preferred variants included hIL18 with 4CS+S10C/N155C and either K53T/V153K (as in XENP44167) and K53T/M60K (as in XENP44161), which as depicted in FIG. 89 are respectively 9X and 88X reduced in activity compared to “WT” (which includes only 4CS+S10C/N155C), in comparison to XENP41431 which is 406X reduced in activity. While the K53T/V153K variants is more potent than the K53T/M60K variant, the latter (as depicted in FIG. 95) is more resistant to IL18BP (individually, V153K improves potency, while subtly improving IL18BP resistance; M60K improves IL18BP resistance but reduces potency).

Further variants were engineered by building on these preferred variants, corresponding data for which are depicted in FIGS. 96-106 and 110.

A two-way MLR assay was used to investigate the activity of the additional IL18 variants. Individual whole PBMCs from two donors were incubated at 1:1 ratio (200K/well) with IL18-Fc fusions of varying potencies for 5 days after which intracellular IFNγ was assessed as an indication of activity, data for which are shown in FIG. 111.

### Example 14: Investigating Further IL18 Variants in Cynomolgus Monkeys

Several of these variants were investigated in cynomolgus studies similar to those described in Example 7C, data for which are depicted in FIG. 112. The data show that active forms of the potency-reduced IL18-Fc fusions exhibit slow receptor-mediated clearance in the cynomolgus monkeys.

### Example 15: Engineering Tumor Antigen (TAA)×CD3×IL18 triAbs

### 15A: Formats

### 15A(a): 1+1+1 Fab-scFv-Fc×IL18-Fc

One format is the “1+1+1 Fab-scFv-Fc×IL18-Fc” format (cartoon schematic depicted in FIG. 118A) which comprises a first monomer comprising a variable heavy (VH) region covalently attached (optionally via a domain linker) to the N-terminus of an scFv covalently attached (optionally via a domain linker) to the N-terminus of a first heterodimeric Fc chain, a second monomer comprising an IL18 monomer covalently attached (optionally via a domain linker) to the N-terminus of a complementary second heterodimeric Fc chain, and a third monomer that is a corresponding light chain VL-CL (wherein the VL forms a binding domain with the VH).

### 15A(b): 1+1+1 scFv-Fc×IL18-Fab-Fc

Another format is the “1+1+1 scFv-Fc×IL18-Fab-Fc” format (cartoon schematic depicted in FIG. 118B) which comprises a first monomer comprising an scFv covalently attached (optionally via a domain linker) to the N-terminus of a first heterodimeric Fc chain, a second monomer comprising an IL18 monomer covalently attached (optionally via a domain linker) to the N-terminus of a VH region covalently attached (optionally via a domain linker) to the N-terminus of a complementary second heterodimeric Fc chain, and a third monomer that is a corresponding light chain VL-CL (wherein the VL forms a binding domain with the VH).

### 15A(c): 1+1+1 IL18-scFv-Fc× Fab-Fc

Another format is the “1+1+1 IL18-scFv-Fc× Fab-Fc” format (cartoon schematic depicted in FIG. 118C) which comprises a first monomer comprising an IL18 covalently attached (optionally via a domain linker) to the N-terminus of an scFv covalently attached (optionally via a domain linker) to the N-terminus of a first heterodimeric Fc chain, a second monomer comprising a VH region covalently attached (optionally via a domain linker) to the N-terminus of a complementary second heterodimeric Fc chain, and a third monomer that is a corresponding light chain VL-CL (wherein the VL forms a binding domain with the VH).

### 15A(d): 1+1+1 Fab-Fc-scFv×IL18-Fc

Another format is the “1+1+1 Fab-Fc-scFv×IL18-Fc” format (cartoon schematic depicted in FIG. 118D) which comprises a first monomer comprising a VH region covalently attached (optionally via a domain linker) to the N-terminus of a first heterodimeric Fc chain covalently attached (optionally via a domain linker) to the N-terminus of an scFv, a second monomer comprising an IL18 monomer covalently attached (optionally via a domain linker) to the N-terminus of a complementary second heterodimeric Fc chain, and a third monomer that is a corresponding light chain VL-CL (wherein the VL forms a binding domain with the VH).

### 15A(e): 2+1+1 Fab-scFv-Fc×IL18-Fab-Fc

Another format is the “2+1+1 Fab-scFv-Fc×IL18-Fab-Fc” format (cartoon schematic depicted in FIG. 118E) which comprises a first monomer comprising a first VH region covalently attached (optionally via a domain linker) to the N-terminus of an scFv covalently attached (optionally via a domain linker) to the N-terminus of a first heterodimeric Fc chain, a second monomer comprising an IL18 monomer covalently attached (optionally via a domain linker) to the N-terminus of a second VH region covalently attached (optionally via a domain linker) to the N-terminus of a complementary second heterodimeric Fc chain, and a third monomer that is a corresponding light chain VL-CL (wherein the VL form binding domains with the VHs).

### 15A(f): 2+1+1 Fab-Fab-Fc×IL18-scFv-Fc

Another format is the “2+1+1 Fab-Fab-Fc×IL18-scFv-Fc” format (cartoon schematic depicted in FIG. 118F) which comprises a first monomer comprising a first VH region covalently attached (optionally via a domain linker) to the N-terminus of a second VH region covalently attached (optionally via a domain linker) to the N-terminus of a first heterodimeric Fc chain, a second monomer comprising an IL18 monomer covalently attached (optionally via a domain linker) to the N-terminus of an scFv covalently attached (optionally via a domain linker) to the N-terminus of a complementary second heterodimeric Fc chain.

### 15A(g): 1+1+1IL18-Fab (via VH)-scFv-Fc×Empty-Fc

Another format is the “1+1+1 IL18-Fab (via VH)-scFv-Fc×empty-Fc” format (cartoon schematic depicted in FIG. 118G) which comprises a first monomer comprising a TL18 monomer covalently attached (optionally via a domain linker) to the N-terminus of a variable heavy (VH) region covalently attached (optionally via a domain linker) to the N-terminus of an scFv covalently attached (optionally via a domain linker) to the N-terminus of a first heterodimeric Fc chain, a second monomer comprising a second heterodimeric Fc chain, and a third monomer that is a corresponding light chain VL-CL (wherein the VL forms a binding domain with the VH).

### 15A(h): 1+1+1 IL18-Fab (via VL)-scFv-Fc×Empty-Fc

Another format is the “1+1+1 IL18-Fab (via VL)-scFv-Fc×empty-Fc” format (cartoon schematic depicted in FIG. 118H) which comprises a first monomer comprising a variable heavy (VH) region covalently attached (optionally via a domain linker) to the N-terminus of an scFv covalently attached (optionally via a domain linker) to the N-terminus of a first heterodimeric Fc chain, a second monomer comprising a second heterodimeric Fc chain, and a third monomer comprising an IL18 monomer covalently attached (optionally via a domain linker) to the N-terminus of a variable light (VL) region covalently attached to the N-terminus of a constant light domain (wherein the VL forms a binding domain with the VH).

### 15B: Antigen Binding Domains

### 15B(a): CD3 Antigen Binding Domains

Sequences for human, mouse, and cynomolgus CD3 are depicted in FIG. 119 and are useful for the development of cross-reactive CD3 antigen binding domains for ease of clinical development. Sequences for CD3 antigen binding domains which may find use in the TAA×CD3×IL18 triAbs of the invention are depicted in FIG. 120.

### 15B(b): B7H3 Binding Domains

Sequences for human, mouse, and cynomolgus B7H3 are depicted in FIG. 121 and are useful for the development of cross-reactive B7H3 antigen binding domains for ease of clinical development. Sequences for B7H3 binding domains which may find use in the B7H3×CD3×IL18 triAbs of the invention are depicted in FIGS. 146, 147, 148, and 149.

### 15B(c): MSLN Binding Domains

Sequences for human, mouse, and cynomolgus MSLN are depicted in FIG. 168 and are useful for the development of cross-reactive MSLN antigen binding domains for ease of clinical development. Sequences for MSLN binding domains which may find use in the MSLN×CD3×IL18 triAbs of the invention are depicted in FIG. 156.

### 15B(d): GPC3 Binding Domains

Sequences for human, mouse, and cynomolgus GPC3 are depicted in FIG. 169 and are useful for the development of cross-reactive GPC3 antigen binding domains for ease of clinical development. Sequences for GPC3 binding domains which may find use in the GPC3×CD3×IL18 triAbs of the invention are depicted in SEQ ID NOS:2422-2537.

### Example 16: TAA×CD3×IL18 triAbs are Active In Vitro

### 16A: B7H3×CD3×IL18 triAbs

### 16A(a): B7H3×CD3×IL18 triAbs in the 1+1+1 Fab-scFv-Fc×IL18-Fc Format Induce Cytokine Secretion

10,000 A431 cancer cells per well were plated and allowed to attach overnight. The following day, PBMC at an E:T of 5:1 were added with the indicated dose of test articles including B7H3×CD3×IL18 triAbs in 1+1+1 Fab-scFv-Fc×IL18-Fc format with either lower potency 4CS/S10C/D37N/K53D/N155C or higher potency 4CS/E6Q/S10C/K53D/N111T/N155C IL18 variants (sequences depicted in FIG. 123) and control molecules (sequences depicted in FIG. 122 and others). Cytokines were assessed via MSD after 3 days. Cytokine secretion was measured using MSD assay (Meso Scale Discovery, Rockville, Md.), data as depicted in FIG. 130.

### 16A(b): B7H3×CD3×IL18 triAbs in the 1+1+1 Fab-Fc×IL18-scFv-Fc Format Induce Cytokine Secretion

10,000 A431 cancer cells per well were plated and allowed to attach overnight. The following day, PBMC at an E:T of 5:1 were added with the indicated dose of test articles including B7H3×CD3×IL18 triAbs in 1+1+1 Fab-Fc×IL18-scFv-Fc format with either lower potency 4CS/S10C/D37N/K53D/N155C or higher potency 4CS/E6Q/S10C/K53D/N111T/N155C IL18 variants (sequences depicted in FIG. 119) and control molecules (sequences depicted in FIG. 116). Cytokines were assessed via MSD after 3 days. Cytokine secretion was measured using MSD assay (Meso Scale Discovery, Rockville, Md.), data as depicted in FIG. 131.

### 16A(c): Activity of B7H3×CD3×IL18 triAbs is Format dependent

In another similar experiment, B7H3×CD3×IL18 triAbs in 1+1+1 Fab-scFv-Fc×IL18-Fc, 1+1+1 Fab-Fc-scFv×IL18-Fc, and 1+1+1 Fab-Fc×IL18-scFv-Fc formats were investigated. Data as depicted in FIG. 132 show that B7H3×CD3×IL18 triAbs in the 1+1+1 Fab-scFv-Fc×IL18-Fc format were more potent than in the 1+1+1 Fab-Fc×IL18-scFv-Fc format which was in turn more potent than in the 1+1+1 Fab-Fc-scFv×IL18-Fc format.

In another experiment, B7H3×CD3×IL18 triAbs in the 1+1+1 scFv-Fc×IL18-Fab-Fc and 1+1+1 IL18-scFv-Fc× Fab-Fc formats were investigated. Data show that both formats were active.

### 16A(d): Activity of B7H3×CD3×IL18 triAbs is CD3 Affinity Dependent

In another similar experiment, B7H3×CD3×IL18 triAbs in various formats and having 30 nM or 5 nM CD3 affinity and with either lower potency 4CS/S10C/D37N/K53D/N155C or higher potency 4CS/E6Q/S10C/K53D/N111T/N155C IL18 variants were investigated. Data as depicted in FIG. 133 show that regardless of format or IL18 potency, higher CD3 affinity increased potency.

### 16A(e): B7H3×CD3×IL18 triAbs Enhance Th2 Response

In another similar experiment, B7H3×CD3×IL18 triAb was compared to IL18-Fc, CD3 bsAb, or IL18-Fc+CD3 bsAb. Data as depicted in FIG. 134 show that IL18-Fc induces Th1 polarization, while the IL18 triAb enhances Th2 response.

### 16A(f): Activity of B7H3×CD3×IL18 triAbs is Enhanced by Exogenous IL-12

In another similar experiment, B7H3×CD3×IL18 triAbs were compared to CD3 bsAb in the absence or presence of exogenous IL-12 (100 ng/ml). Data as depicted in FIG. 135 show that induction of IFNγ secretion by the test articles was enhanced by addition of exogenous IL-12.

### 16A(g): Activity of B7H3×CD3×IL18 triAbs is IL18 Potency Dependent

In another experiment, B7H3×CD3×IL18 triAbs in the 1+1+1 Fab-scFv-Fc×IL18-Fc format and having IL18 variants of differing potencies (as described in Example 11; 4CS_S10C/K53T/V153K/N155C>4CS_S10C/K53T/M60K/N155C>4CS_E6Q/S10C/K53D/N111T/N155C) were investigated. Data show that higher potency IL18 arms improve potency of IFNγ secretion.

### 16A(h): B7H3×CD3×IL18 triAbs Kill Tumor Cells In Vitro

Fresh PBMCs or prestimulated PBMCs (stimulated with plate bound anti-CD3 (0.5 μg/ml) and anti-CD28 (1 μg/ml) for 3 days) were seeded in the presence of OVCAR8 cells (E:T 1:10) and dose titration of indicated B7H3×CD3×IL18 triAbs. RTCC was assessed after 3 days via luciferase assay system. Data show that the triAbs induced killing of OVCAR8 cells, and as above with IFNγ secretion, potency correlated with IL18 variant potencies.

### 16B: MSLN×CD3×IL18 triAbs

### 16B(a): MSLN×CD3×IL18 triAbs are Active in Various Formats

In another similar experiment, MSLN×CD3×IL18 triAbs in the 1+1+1 Fab-scFv-Fc×IL18-Fc, 1+1+1 scFv-Fc×IL18-Fab-Fc, and 2+1+1 Fab-scFv-Fc×IL18-Fab-Fc formats were investigated. Data as depicted in FIG. 136 show that each of the triAbs induced IFNγ secretion, although the 1+1+1 Fab-scFv-Fc×IL18-Fc and 2+1+1 Fab-scFv-Fc×IL18-Fab-Fc formats were more potent than the 1+1+1 scFv-Fc×IL18-Fab-Fc format.

### 16B(b): MSLN×CD3×IL18 triAbs are Active Only in the Presence of Cancer Cells

In another similar experiment, MSLN×CD3×IL18 triAbs were investigated in the presence or absence of OVCAR8 cancer cells. Data as depicted in FIG. 137 show that the triAbs are only active in the presence of cancer cells.

### 16B(c): MSLN×CD3×IL18 triAbs Activity May be Modulated by Tuning IL18 and CD3 Potencies

In a first experiment, MSLN×CD3×IL18 triAbs in the 1+1+1 scFv-Fc×IL18-Fab-Fc or 1+1+1 IL18-scFv-Fc× Fab-Fc formats and varying IL18 potencies were investigated. The triAbs were incubated with prestimulated T cell and OVCAR8 (1:1 E:T). Data depicting IFNγ secretion (FIG. 165) demonstrate that swapping IL18 potencies (4CS/S10C/K53T/V153K/N155C>4CS/S10C/K53T/M60K/N155C>4CS/E6Q/S10C/K53D/N111T/N155C) modulated triAb activity, particularly in the context of 1+1+1 scFv-Fc×IL18-Fab-Fc format.

Next, MSLN×CD3×IL18 triAbs in the 1+1+1 Fab-scFv-Fc×IL18-Fc format and varying IL18 potencies and CD3 binding affinities were investigated. As above, triAbs were incubated with prestimulated T cell and OVCAR8 (1:1 E:T). Data depicting IFNγ secretion (FIG. 166) demonstrate that swapping CD3 binding affinity modulated triAb activity; and that effect of swapping IL18 potencies was more pronounced in the context of lower CD3 affinities.

Finally, to identify which combination of IL18 potency and CD3 affinity was optimal, triAbs were incubated with prestimulated T cell and OVCAR8 at 1:1 E:T and 1:10 E:T. Data depicting IFNγ secretion (FIG. 167) demonstrate that surprisingly, the med-potency hIL18 (4CS/S10C/K53T/M60K/N155C) variant in combination with 30 nM CD3 affinity in the 1+1+1 Fab-scFv-Fc×IL18-Fc format best maintained efficacy at low E:T ratio.

### 16C: GPC3×CD3×IL18 triAbs

### 16C(a): GPC3×CD3×IL18 triAbs are Active

20K tumor cells were mixed with PBMC (E:T 5:1) or purified T cells (E:T 1:1) and incubated with varying concentrations of indicated test articles (including GPC3×CD3×IL18 triAbs in the 1+1+1 Fab-scFv-Fc×IL18-Fc and 1+1+1 scFv-Fc×IL18-Fab-Fc formats) for 48 hr at 37° C. Cells were stained with flow Ab for 45 min at 4° C. followed by fixation & analysis by FACs. Data as depicted in FIG. 138 show that the GPC3×CD3×IL18 triAbs induced cancer cell kill, T cell activation, NK cell activation, and cytokine secretion.

### 16C(b): GPC3×CD3×IL18 triAbs are Active in Multiple Formats

3K tumor cells were mixed with IL-12 treated PBMC (E:T 1:1), incubated with varying concentrations of IL18×GPC3×CD3 in 1+1+1 Fab-scFv-Fc×IL18-Fc, 2+1+1 Fab-Fab-Fc×IL18-scFv-Fc, and 2+1+1 Fab-scFv-Fc×IL18-Fab-Fc formats for 5 days at 37° C. Tumor cells killing was determined by luciferase assay. Data as depicted in FIG. 139 show triAbs in each of the formats induced cancer cell kill.

Notably as depicted in FIG. 138, XENP43374 in the 1+1+1 scFv-Fc×IL18-Fab-Fc format is much less potent than XENP42989 in the 1+1+1 Fab-scFv-Fc×IL18-Fc format. In fact, XENP43374 is less potent than corresponding GPC3×CD3 XENP35843. In another experiment, 50K GPC3⁺ HepG2 cells were incubated with varying concentrations of the triAbs for 1 hour at 4° C., followed by wash and incubation with secondary antibody to detect binding. Data for which are depicted in FIG. 162 show that XENP43374 (and XENP43375) in the 1+1+1 scFv-Fc×IL18-Fab-Fc format had decreased cell binding. Without wishing to be bound by theory, it was hypothesized that positioning of the IL18 on the GPC3 Fab sterically modulated GPC3 binding.

### 16D: Additional Mechanistic Observations

PBMCs were seeded in the presence of OVCAR8 cells (E:T 10:1) and either B7H3×CD3×IL18 triAbs or RSV×CD3×IL18 triAbs. IFNγ was measured after 3 days via MSD and IL18Rα and Ki67 were assessed after 3 days via flow cytometry, data for which are depicted in FIG. 145. The data who that the non-tumor targeting triAbs (RSV×CD3×IL18) drive activation and proliferation of IL18Rα+ T cells, but do not enhance IFNγ secretion suggesting that the tumor-targeted triAbs may avoid cytokine release outside the tumor environment.

In another experiment, 50K purified T cells or 250K PGMCs were incubated with GPC3×CD3×IL18 or B7H3×CD3×IL18 triAbs in the absence of target cells for 72 hours at 37° C., after which cell supernatant was harvested to analyze cytokine secretion. Data as depicted in FIG. 163 show that IL18 potentiates CD3 dependent IFNγ secretion from T cells and PBMCs.

In yet another experiment, 20K tumor cells were mixed with PBMC (E:T 5:1) or purified T cells (E:T 1:1) and incubated with GPC3×CD3×IL18 triAbs for 48 hours at 37° C. after which cells were stained to determine cell kill. The data as depicted in FIG. 164 show that IL12 combines with IL18 to potentiate CD3 mediated tumor cell killing. This observation is best demonstrated by XENP43374 which as described above suffered from diminished potency.

### Example 17: TAA×CD3×IL18 triAbs are Active In Vivo

### 17A: B7H3×CD3×IL18 triAbs Increase Antitumor Activity In Vivo

CD34+ Hu-NSG, which are NSG mice engrafted with human CD34+ hematopoietic stem cells so as to develop a functional human immune system with no reactivity towards the host were obtained from The Jackson Laboratory (Bar Harbor, Maine), were used. On Day −21, mice were intradermally inoculated with 3×106 pp65-MCF7-gfp cells. Mice were then treated intraperitoneally on Days 0 and 7 with B7H3×CD3×IL18 triAbs or B7H3×CD3 or PBS control. Tumor volumes were monitored by caliper measurements, data for which are shown (days post 1st dose). By Day 3 (data not shown), the triAbs enhanced antitumor activity in comparison to PBS control. By Day 10, as depicted in FIG. 140, the triAbs enhanced antitumor activity in comparison to PBS and B7H3×CD3 control.

### 17B: GPC3×CD3×IL18 triAbs Increase Antitumor Activity In Vivo

In another study, NSG mice that were MHC I/II-DKO (NSG-DKO) and thus resistant to GVHD were used. On Day −14, mice were intradermally inoculated with 2×10⁶ HepG2 tumor cells. Mice were then intraperitoneally injected with 5×10⁶ human PBMCs and treated with the indicated test articles/test article combinations (GPC3×CD3×IL18 triAbs or GPC3×CD3 (alone or in combination with IL18-Fc) or PBS control) on Day 0, and then further treated on Days 0, 7, 14, and 21. The GPC3×CD3×IL18 triAbs use different IL18 variants ranking in potency as follows: 4CS/S10C/K53T/V153K/N155C>4CS/S10C/K53T/M60K/N155C>4CS/E6Q/S10C/K53D/N111T/N155C>4CS/S10C/E31Q/D37N/N41Q/K53D/N155C. Tumor volumes were monitored by caliper measurements, data for which are shown (days post 1^{st} dose).

1. A construct comprising a variant human IL-18 protein as compared to SEQ ID NO: 2, wherein the variant comprises an amino acid modification selected from the group consisting of K8R, K8D, K8N, K8S, K8T, I49D, I49E, I49N, I49Q, I49Y, I49F, M60R, Q103Y, Q103E, Q103K, Q103R, V153E, V153K, V153R, V153Y, V153Q, V153N, V153D, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53 T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111 T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, wherein 4CS comprises the amino acid substitutions C38S/C68S/C76S/C127S, wherein said variant human IL-18 protein has reduced binding to IL-18BP.

2. The construct according to claim 1, wherein said variant human IL-18 protein has amino acid substitutions selected from 4CS/S10C/K53T/M60K/N155C and 4CS/S10C/K53T/V153K/N155C.

3. The construct according to claim 1, wherein said construct comprises the amino acid sequence of SEQ ID NO: 29.

4. A construct comprising:
(a) a first antigen binding domain (ABD) that binds to the extracellular domain (ECD) of a human tumor target antigen (TTA);
(b) a second antigen binding domain (ABD) that binds to the extracellular domain (ECD) of human CD3ε; and
(c) a variant human IL-18 protein, wherein said variant human IL-18 protein has reduced binding to IL-BP.

5. The construct according to claim 4, wherein said construct comprises:
(a) a first monomer comprising from N-terminus to C-terminus: a first variable heavy domain (VH1), an scFv, and a first Fc domain;
(b) a second monomer comprising from N-terminus to C-terminus: said variant human IL18 protein and a second Fc domain; and
(c) a third monomer comprising from N-terminus to C-terminus: a first variable light domain (VL1) and a constant light domain,
wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

6. The construct according to claim 4, wherein said construct comprises:
(a) a first monomer comprising from N-terminus to C-terminus: an scFv and a first Fc domain;
(b) a second monomer comprising from N-terminus to C-terminus: said variant human IL18 protein, a first variable heavy domain (VH1), and a second Fc domain; and
(c) a third monomer comprising from N-terminus to C-terminus: a first variable light domain (VL1) and a constant light domain,
wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

7. The construct according to claim 4, wherein said construct comprises:
(a) a first monomer comprising from N-terminus to C-terminus: said variant human IL18 protein, an scFv, and a first Fc domain;
(b) a second monomer comprising from N-terminus to C-terminus: a first variable heavy domain (VH1) and a second Fc domain; and
(c) a third monomer comprising from N-terminus to C-terminus: a first variable light domain (VL1) and a constant light domain,
wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

8. The construct according to claim 4, wherein said construct comprises:
(a) a first monomer comprising from N-terminus to C-terminus: a first variable heavy domain (VH1), a first Fc domain, and an scFv;
(b) a second monomer comprising from N-terminus to C-terminus: said variant human IL-18 protein and a second Fc domain; and
(c) a third monomer comprising from N-terminus to C-terminus: a first variable light domain (VL1) and a constant light domain,
wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

9. The construct according to claim 4, wherein said construct comprises:
(a) a first monomer comprising from N-terminus to C-terminus: a first variable heavy domain (VH1), an scFv, and a first Fc domain;
(b) a second monomer comprising from N-terminus to C-terminus: said variant human IL-18 protein, a second variable heavy domain (VH2), and a second Fe domain; and
(c) a third monomer comprising from N-terminus to C-terminus: a first variable light domain (VL1) and a constant light domain,
wherein the VH1 and the VL1 form the first ABD that binds to the ECD of the human TTA, and wherein the VH2 and the VL1 form another ABD to the ECD of the human TTA, and wherein the scFv comprises a third variable heavy domain (VH3), a scFv linker, and a second variable light domain (VL2), wherein the VH3 and the VL2 together form the second ABD that binds to the ECD of human CD3ε.

10. The construct according to claim 4, wherein said construct comprises:
(a) a first monomer comprising from N-terminus to C-terminus: said variant human IL-18 protein, an scFv, and a first Fc domain;
(b) a second monomer comprising from N-terminus to C-terminus: a first variable heavy domain (VH1), a second variable heavy domain (VH2), and a second Fc domain; and
(c) a third monomer comprising from N-terminus to C-terminus: a first variable light domain (VL1) and a constant light domain,
wherein the VH1 and the VL1 form the first ABD that binds to the ECD of the human TTA, and wherein the VH2 and the VL1 form another ABD that binds to the ECD of the human TTA, and wherein the scFv comprises a third variable heavy domain (VH3), a scFv linker, and a second variable light domain (VL2), wherein the VH3 and the VL2 together form the second ABD that binds to the ECD of human CD3ε.

11. The construct according to claim 4, wherein said construct comprises
(a) a first monomer comprising from N-terminus to C-terminus: said variant human IL-18 protein, a first variable heavy domain (VH1), an scFv, and a first Fc domain;
(b) a second monomer comprising a second Fc domain; and
(c) a third monomer comprising from N-terminus to C-terminus: a first variable light domain (VL1) and a constant light domain,
wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

12. The construct according to claim 4, wherein said construct comprises
(a) a first monomer comprising from N-terminus to C-terminus: a first variable heavy domain (VH1), an scFv, and a first Fc domain;
(b) a second monomer comprising a second Fc domain; and
(c) a third monomer comprising from N-terminus to C-terminus: said variant human IL-18 protein, a first variable light domain (VL1), and a constant light domain,
wherein the VH1 and the VL1 together form the first ABD that binds to the ECD of the human TTA and wherein the scFv comprises a second variable heavy domain (VH2), a scFv linker, and a second variable light domain (VL2), wherein the VH2 and the VL2 together form the second ABD that binds to the ECD of human CD3ε.

13. The construct according to claim 4, wherein said variant human IL-18 protein has amino acid modification selected from the group consisting of K8RC K8D, K8N, K8S, K8T, I49D, I49E, I49N, I49Q, I49Y, I49F, M60R, Q103Y, Q103E, Q103K, Q103R, V153E, V153K, V153R, V153Y, V153Q, V153N, V153D, K8R/K53T, K8D/K53T, K8N/K53T, K8S/K53T, K8T/K53T, I49D/K53T, I49E/K53T, I49N/K53T, I49Q/K53T, S49Y/K53T, I49F/K53T, K53T/M60R, K53T1M60K, K53T/Q03Y, K53T/Q13E, K53T/Q153K, K53T/Q103R, K53T/V153E, K53T/V153K, K53T/V153R, K53T/V153Y, K53T/V153Q, K53T/V153N, K53T/V153D, K53T/M6R/V153K, 149Q/K53T/V153K, K53T/Q103R/V153K, K53T/M65K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S1C/K53T/N155C, K8S/S10C/K53T/N155C, K8T/S1C/K53T/N155C, S10C/149D2K53T/N155C, S10C/I49E/K53T/N155C, S1C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53 T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111 T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111T/V153K/N155C, 4CS/E6Q/S10C/M51I/K53T/M60K/N111 T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, and 4CS/E6Q/S10C/K53D/N111T/N155C, wherein 4CS comprises the amino acid substitutions C38S/C68S/C76S/C127S.

14. The construct according to claim 4, wherein said human TTA is selected from the group consisting of EGFR, Trop2, CD20, B7H3, FLT3, CD19, CD123, CD22, CD38, CEA, MSLN, BCMA, CAIX, CLDN18.2, HER2, PD-1, and ANO1.

15. The construct according to claim 4, wherein said first ABD that binds to the ECD of human EGFR comprises a variable heavy domain and variable light domain pair selected from those depicted in FIGS. 150A-150C as SEQ ID NOS: 3386-3409, said first ABD that binds to the ECD of human Trop2 comprises a variable heavy domain and variable light domain pair selected from those depicted in FIGS. 157A-157R as SEQ ID NOS: 3857-4018, said first ABD that binds to the ECD of human CD20 comprises a variable heavy domain and variable light domain pair selected from those depicted in FIG. 153A as SEQ ID NOS: 3432-3441, said first ABD that binds to the ECD of human B7H3 comprises a variable heavy domain and variable light domain pair selected from those depicted in FIGS. 146, 147, 148A-148H, 149A-149R as SEQ ID NOS: 2848-3385, said first ABD that binds to the ECD of human FLT3 comprises a variable heavy domain and variable light domain pair selected from those depicted in FIG. 155 as SEQ ID NOS: 3468-3475, said first ABD that binds to the ECD of human CD19 comprises a variable heavy domain and variable light domain pair selected from those depicted in FIG. 152 as SEQ ID NOS: 3424-3431, said first ABD that binds to the ECD of human CD123 comprises a variable heavy domain and variable light domain pair selected from those depicted in FIG. 154B as SEQ ID NOS: 3464-3467, said first ABD that binds to the ECD of human CEA comprises a variable heavy domain and variable light domain pair selected from those depicted in FIGS. 158A-158C as SEQ ID NOS: 4019-4046, said first ABD that binds to the ECD of human MSLN comprises a variable heavy domain and variable light domain pair selected from those depicted in FIGS. 156A-156Z and 156AA-156FF as SEQ ID NOS: 3467-3856, said first ABD that binds to the ECD of human BCMA comprises a variable heavy domain and variable light domain pair selected from those depicted in FIGS. 160A-160C as SEQ ID NOS: 4054-4115, said first ABD that binds to the ECD of human CAIX comprises a variable heavy domain and variable light domain pair selected from those depicted in FIG. 154A as SEQ ID NOS: 3462-3463, said first ABD that binds to the ECD of human CLDN18.2 comprises a variable heavy domain and variable light domain pair selected from those depicted in FIG. 159 as SEQ ID NOS: 4047-4053, said first ABD that binds to the ECD of human HER2 comprises a variable heavy domain and variable light domain pair selected from those depicted in FIG. 151A-151B as SEQ ID NOS: 3410-3423, or said first ABD that binds to the ECD of human PD-1 comprises a variable heavy domain and variable light domain pair selected from those depicted in FIGS. 161A-161G as SEQ ID NOS: 4116-4155.

16.-28. (canceled)

29. The construct according to claim 4, wherein said second ABD that binds to the ECD of human CD3ε comprises a variable heavy domain and variable light domain pair selected from those depicted in FIGS. 120A-120F as SEQ ID NOS: 1545-1664.

30. A monovalent Fc fusion protein comprising:
(a) a first monomer comprising from N-terminus to C-terminus: a variant human IL-18 protein and a first Fe domain,
wherein said variant human IL-18 protein as compared to SEQ ID NO: 2 comprises an amino acid modification selected from the group consisting of K8R, K8D, K8N, K8S, K8T, I49D, 149E, I49N, I49Q, I49Y, I49F, M60R, Q103Y, Q103E, Q103K, Q103R, V153E, V153K, V153R, V153Y, V153Q, V153N, V153D, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, wherein 4CS comprises the amino acid substitutions C38S/C68S/C76S/C127S, wherein said variant human IL-18 protein has reduced binding to IL-18BP; and
(b) a second monomer comprising a second Fc domain.

31. The monovalent Fc fusion protein according to claim 30, wherein said variant human IL-18 protein has amino acid substitutions selected from 4CS/S10C/K53T/M60K/N155C and 4CS/S10C/K53T/V153K/N155C.

32. A Fab-Fc fusion protein comprising:
(a) a first monomer comprising from N-terminus to C-terminus: a variable heavy (VH) chain and a first Fc domain;
(b) a second monomer comprising from N-terminus to C-terminus: a variant human IL-18 protein and a second Fc domain,
wherein said variant human IL-18 protein as compared to SEQ ID NO: 2 comprises an amino acid modification selected from the group consisting of, K8R, K8D, K8N, K8S, K8T, I49D, 149E, I49N, I49Q, I49Y, I49F, M60R, Q103Y, Q103E, Q103K, Q103R, V153E, V153K, V153R, V153Y, V153Q, V153N, V153D, K8R/K53T, K8D/K53T, K8N/K53T, K8S/K53T, K8T/K53T, I49D/K53T, I49E/K53T, I49N/K53T, I49Q/K53T, I49Y/K53T, I49F/K53T, K53T/M60R, K53T/M60K, K53T/Q103Y, K53T/Q103E, K53T/Q103K, K53T/Q103R, K53T/V153E, K53T/V153K, K53T/V153R, K53T/V153Y, K53T/V153Q, K53T/V153N, K53T/V153D, K53T/M60R/V153K, I49Q/K53T/V153K, K53T/Q103R/V153K, K53T/M60K/V153K, K53T/M60K/N111T/V153K, M51I/K53T/M60K/N111T/V153K, E6Q/K53T/M60K/V153K, E6Q/K53T/M60K/N111T/V153K, E6Q/M51I/K53T/M60K/N111T/V153K, E6Q/K53T/V153K, M51I/K53T/V153K, K53T/N111T/V153K, E6Q/K53T/N111T/V153K, M51I/K53T/N111T/V153K, E6Q/M51I/K53T/V153K, K8R/S10C/K53T/N155C, K8D/S10C/K53T/N155C, K8N/S10C/K53T/N155C, K8S/S10C/K53T/N155C, K8T/S10C/K53T/N155C, S10C/I49D/K53T/N155C, S10C/I49E/K53T/N155C, S10C/I49N/K53T/N155C, S10C/I49Q/K53T/N155C, S10C/I49Y/K53T/N155C, S10C/I49F/K53T/N155C, S10C/K53T/M60R/N155C, S10C/K53T/M60K/N155C, S10C/K53T/Q103Y/N155C, S10C/K53T/Q103E/N155C, S10C/K53T/Q103K/N155C, S10C/K53T/Q103R/N155C, S10C/K53T/V153E/N155C, S10C/K53T/V153K/N155C, S10C/K53T/V153R/N155C, S10C/K53T/V153Y/N155C, S10C/K53T/V153Q/N155C, S10C/K53T/V153N/N155C, S10C/K53T/V153D/N155C, S10C/N155C/H6-12, S10C/K53T/N155C, S10C/K53T/M60R/V153K/N155C, S10C/I49Q/K53T/V153K/N155C, S10C/K53T/Q103R/V153K/N155C, S10C/K53T/M60K/V153K/N155C, S10C/K53T/M60K/N111T/V153K/N155C, S10C/M51I/K53T/M60K/N111T/V153K/N155C, E6Q/S10C/K53T/M60K/V153K/N155C, E6Q/S10C/K53T/M60K/N111T/V153K/N155C, E6Q/S10C/M51I/K53T/M60K/N111T/V153K/N155C, E6Q/S10C/K53T/V153K/N155C, S10C/M51I/K53T/V153K/N155C, S10C/K53T/N111T/V153K/N155C, E6Q/S10C/K53T/N111T/V153K/N155C, S10C/M51I/K53T/N111T/V153K/N155C, E6Q/S10C/M51I/K53T/V153K/N155C, 4CS/K8R/S10C/K53T/N155C, 4CS/K8D/S10C/K53T/N155C, 4CS/K8N/S10C/K53T/N155C, 4CS/K8S/S10C/K53T/N155C, 4CS/K8T/S10C/K53T/N155C, 4CS/S10C/I49D/K53T/N155C, 4CS/S10C/I49E/K53T/N155C, 4CS/S10C/I49N/K53T/N155C, 4CS/S10C/I49Q/K53T/N155C, 4CS/S10C/I49Y/K53T/N155C, 4CS/S10C/I49F/K53T/N155C, 4CS/S10C/K53T/M60R/N155C, 4CS/S10C/K53T/M60K/N155C, 4CS/S10C/K53T/Q103Y/N155C, 4CS/S10C/K53T/Q103E/N155C, 4CS/S10C/K53T/Q103K/N155C, 4CS/S10C/K53T/Q103R/N155C, 4CS/S10C/K53T/V153E/N155C, 4CS/S10C/K53T/V153K/N155C, 4CS/S10C/K53T/V153R/N155C, 4CS/S10C/K53T/V153Y/N155C, 4CS/S10C/K53T/V153Q/N155C, 4CS/S10C/K53T/V153N/N155C, 4CS/S10C/K53T/V153D/N155C, 4CS/S10C/N155C/H6-12, 4CS/S10C/K53T/N155C, 4CS/S10C/K53T/M60R/V153K/N155C, 4CS/S10C/I49Q/K53T/V153K/N155C, 4CS/S10C/K53T/Q103R/V153K/N155C, 4CS/S10C/K53T/M60K/V153K/N155C, 4CS/S10C/K53T/M60K/N111T/V153K/N155C, 4CS/S10C/M51I/K53T/M60K/N111T/V153K/N155C, 4CS/E6Q/S10C/K53T/M60K/V153K/N155C, 4CS/E6Q/S10C/K53T/M60K/N111 T/V153K/N155C, 4CS/E6Q/S10C/M51I/K53T/M60K/N111T/V153K/N155C, 4CS/E6Q/S10C/K53T/V153K/N155C, 4CS/S10C/M51I/K53T/V153K/N155C, 4CS/S10C/K53T/N111T/V153K/N155C, 4CS/E6Q/S10C/K53T/N111T/V153K/N155C, 4CS/S10C/M51I/K53T/N111T/V153K/N155C, and 4CS/E6Q/S10C/M51I/K53T/V153K/N155C, wherein 4CS comprises the amino acid substitutions C38S/C68S/C76S/C127S, wherein said variant human IL-18 protein has reduced binding to IL-18BP; and
(c) a third monomer comprising a variable light (VL) chain, wherein the VH and VL form an antigen binding fragment (Fab).

33. The Fab-Fc fusion protein according to claim 32, wherein said variant human IL-18 protein has amino acid substitutions selected from 4CS/S10C/K53T/M60K/N155C and 4CS/S10C/K53T/V153K/N155C.
